(19) United States
(12) Patent Application Publication
(10) Pub. No.: US 2024/0262848 A1
(43) Pub. Date: Aug. 8, 2024

(54) HETEROCYCLIC COMPOUNDS, COMPOSITIONS THEREOF, AND METHODS OF TREATMENT THEREWITH

(71) Applicant: BeiGene, Ltd., Grand Cayman (KY)

(72) Inventors: Chao YU, Beijing (CN); Jie CHEN, Beijing (CN); Yichao BIAN, Beijing (CN); Xiaoyu LI, Beijing (CN); Hanzi SUN, Beijing (CN); Huaqing LIU, Beijing (CN); Ce WANG, Beijing (CN); Zhiwei WANG, Beijing (CN)

(73) Assignee: BeiGene, Ltd., Grand Cayman (KY)

(21) Appl. No.: 18/597,844

(22) Filed: Mar. 6, 2024

Related U.S. Application Data
(63) Continuation of application No. PCT/CN2023/112168, filed on Aug. 10, 2023.

(30) Foreign Application Priority Data
Aug. 11, 2022 (WO) PCT/CN2022/111871
Sep. 23, 2022 (WO) PCT/CN2022/121125
Mar. 1, 2023 (WO) PCT/CN2023/079087
May 10, 2023 (WO) PCT/CN2023/093212

(51) Int. Cl. C07D 519/00; A61K 31/519; A61K 31/553; A61P 35/00; C07D 498/22

(52) U.S. Cl. CPC C07D 519/00; A61K 31/519; A61K 31/553; A61P 35/00; C07D 498/22

(57) ABSTRACT

Provided herein are compounds having the following structure:

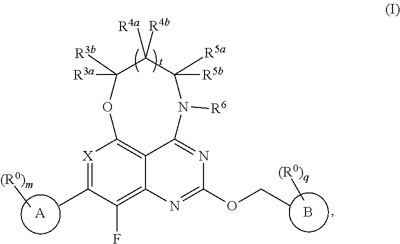

wherein the substituents are as defined herein, compositions comprising an effective amount of a compound, and methods for modulating activity of KRAS G12D and/or G12V.

### FIELD

Provided herein are heterocyclic compounds useful for treating cancer, a pharmaceutical composition comprising the compounds and, methods of using the compounds for treating cancer or a condition treatable or preventable by inhibition of KRAS activity, comprising administering an effective amount of the compounds to a subject in need thereof.

### BACKGROUND

Ras is a family of proteins which are associated with cell membrane through their C-terminal membrane targeting region and well known as the molecular switch in intracellular signaling network (Cox A D, Der CJ. Ras history: The saga continues. Small GTPases. 2010; 1(1):2-27). Ras proteins bind with either GTP or GDP and switch between “on” and “off” states. When Ras proteins bind with GDP, it is in the off (or inactive) state. And when Ras is switched on by certain growth promoting stimuli like growth factors, Ras proteins will be induced to exchange its bound GDP for a GTP and turn into on (or active) state (Malumbres M, Barbacid M. RAS oncogenes: the first 30 years. Nat Rev Cancer. 2003; 3(6):459-465). By switching to active state, Ras protein can interact with different downstream proteins and activate related signaling pathways (Berndt N, Hamilton A D, Sebti S M. Targeting protein prenylation for cancer therapy. Nat Rev Cancer. 2011; 11(11):775-791). Ras superfamily contains different subfamilies including Ras, Ral, Rap, Rheb, Rad, Rit and Miro (Wennerberg K, Rossman K L, Der CJ. The Ras superfamily at a glance. J Cell Sci. 2005; 118(Pt 5): 843-846). HRas, NRas and KRas are the most well studied proteins in Ras family since these proteins are the most common oncogenes in human cancers (O'Bryan JP. Pharmacological targeting of RAS: Recent success with direct inhibitors. Pharmacol Res. 2019; 139:503-511).

KRas is one of the most frequently mutated genes in human cancers. Based on data from Catalogue of Somatic Mutations (COSMIC) database, KRas mutation can be found in about 20% of human cancers, including pancreatic cancer, colorectal cancer, lung cancer, skin cancer etc. (O'Bryan JP. Pharmacological targeting of RAS: Recent success with direct inhibitors. Pharmacol Res. 2019; 139:503-511). The most common KRas mutations are found at position G12 and G13 by blocking the GTPase activating proteins (GAP) stimulated GTP hydrolysis activity of KRas (Wang W, Fang G, Rudolph J. Ras inhibition via direct Ras binding—is there a path forward ?. Bioorg Med Chem Lett. 2012; 22(18):5766-5776). That results in the over activation of KRas protein and ultimately leads to uncontrolled cell proliferation and cancer.

Among different cancers, pancreatic cancer is considered as the most KRas-addicted cancer type. KRas mutation is found in 94.1% of pancreatic ductal adenocarcinoma (PDAC). G12D (41%) and G12V (34%) mutations of KRas are the two most predominant mutations in all the KRas mutated PDAC (Waters A M, Der CJ. KRAS: The Critical Driver and Therapeutic Target for Pancreatic Cancer. Cold Spring Harb Perspect Med. 2018; 8(9):a031435). In vivo data generated by mouse models proves that the progression and maintenance of pancreatic cancer are highly rely on the constitutive activation of KRas downstream signaling (Siveke J T, Schmid R M. Chromosomal instability in mouse metastatic pancreatic cancer—it's Kras and Tp53 after all. Cancer Cell. 2005; 7(5):405-407). Which indicates that mutated KRas protein is a highly attractive drug target for pancreatic cancer and also other cancers with KRas mutation. Since WT KRas protein also plays a critical role in the function of normal tissue and WT KRas function is demonstrated to be essential for adult hematopoiesis (Malumbres M, Barbacid M. RAS oncogenes: the first 30 years. Nat Rev Cancer. 2003; 3(6):459-465). It is highly deserved that a potential drug molecule can selectively inhibit mutated Kras protein in cancer cells and spare its W′T companion in normal cells.

Thus, KRas G12D and G12V mutations are a highly attractive target for cancer and other cancers with this mutation. As such, small-molecule therapeutic agents that are capable to selectively bind with Kras G12D or G12V and inhibit its function would be very useful.

Citation or identification of any reference in this section of this application is not to be construed as an admission that the reference is prior art to the present application.

### SUMMARY

Provided herein are compounds having the following formula (I):

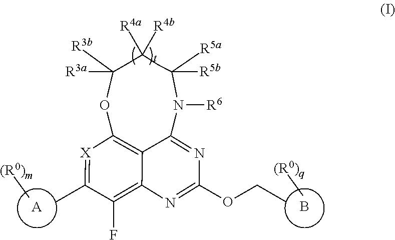

and pharmaceutically acceptable salts, tautomers, stereoisomers, enantiomers, atropisomers, isotopologues, and prodrugs thereof, wherein the substituents are as defined herein.

In one embodiment, the compound is selected from Table 1-Table 5.

In one embodiment, provided herein is a method for inhibiting the activity of KRAS mutant protein or KRAS amplification in a cell, comprising contacting said cell with an effective amount of a compound provided herein, or a pharmaceutically acceptable salt, tautomer, isotopologue, stereoisomer, enantiomer, atropisomer, or prodrug thereof, optionally wherein the KRAS mutant protein is KRAS G12D and/or G12V mutant protein.

In one embodiment, provided herein is a method for treatment or prevention of cancer, the method comprising administering to a subject in need thereof an effective amount of a compound provided herein, or a pharmaceutically acceptable salt, tautomer, isotopologue, stereoisomer, enantiomer, atropisomer, or prodrug thereof, optionally wherein the cancer is mediated by KRAS mutation; preferably KRAS G12D and/or G12V mutation.

### DETAILED DESCRIPTION

### Definitions

As used herein, “KRAS gene” refers to a gene selected from the group consisting of: DIRAS1; DIRAS2; DIRAS3; ERAS; GEM; HRAS; KRAS; MRAS; NKIRAS1; NKIRAS2; NRAS; RALA; RALB; RAPIA; RAPIB; RAP2A; RAP2B; RAP2C; RASD1; RASD2; RASLIOA; RASLIOB; RASL11A; RASL11B; RASL12; REM1; REM2; RERG; RERGL; RRAD; RRAS; RRAS2, and mutants thereof.

As used herein, “KRAS protein” refers to a protein or an isoform thereof expressed by a KRAS gene (Scolnick EM, Papageoege AG, Shih TY (1979), “Guanine nucleotide-binding activity for src protein of rat-derived murine sarcoma viruses,” Proc Natl Acad Sci USA. 76 (5): 5355-5559; Kranenburg O(November 2005) “The KRAS oncogene: past, present, and future,” Biochimica et Biophysica Acta (BBA)—Reviews on Cancer, 1756 (2): 81-2).

As used herein, “G12D mutation” refers to the mutation of the 12^{th} amino acid residue located in the G domain of KRAS protein from glycine to aspartic acid.

As used herein, “KRAS G12D” or “G12D” refer to KRAS protein with G12D mutation.

As used herein, “G12V mutation” refers to the mutation of the 12^{th} amino acid residue located in the G domain of KRAS protein from glycine to a valine.

As used herein, “KRAS G12V” or “G12V” refer to KRAS protein with G12V mutation.

As used herein, “KRAS amplification” or “KRAS gene amplification” refer to a genetic alteration that increases the number of copies of the KRAS gene in some cancer cells. This can lead to higher expression and activity of the KRAS protein, which is involved in cell growth and survival. KRAS amplification is found in some types of cancer, such as lung, breast, esophageal, ovarian and testicular cancers.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to +0.2º 20 (or +0.2 degree 20) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include-methyl,-ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)—CH₂, —C(CH₃)—CH(CH₃), —C(CH₂CH₃)—CH₂, —C—CH, —C≡C(CH₃), —C≡C(CH₂CH₃), -CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or ((alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂C₈)alkenyls include -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3octenyl and the like. The double bond of an alkenyl group can be unconjugated or conjugated to another unsaturated group. An alkenyl group can be unsubstituted or substituted.

An “alkynyl” group refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., (2-4 alkynyl. Examples of alkynyl moieties include, but are not limited to ethynyl, propynyl, and butynyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1,1,1]pentyl, bicyclo[2,1,1]hexyl, bicyclo[2,2,1]heptyl, bicyclo[2,2,2]octyl, adamantyl and the like. Examples of unsaturated cycloalkyl groups include cyclohexenyl, cyclo pentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “bridged” bicyclic ring system includes two rings sharing three, four, or five adjacent ring atoms. As used herein, the term “bridge” refers to an atom or chain of atoms that connects two different parts of a molecule. Two atoms connected through a bridge (usually but not always two tertiary carbon atoms) are called “bridgeheads”. In addition to the bridge, the two bridgeheads are connected by at least two individual atoms or atomic chains. Examples of bridged bicyclic ring systems include adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2, Examples include, but are not limited to, 6-dioxa-tricyclo[3.3.1.03,7]nonyl. In one embodiment, the bridge is unsubstituted or substituted —(CH₂)ₙ—, wherein n is 1, 2, 3, 4, or 5. In one embodiment, the bridge is —CH₂—. In one embodiment, the bridge is —(CH₂)₂—. In one embodiment, the bridge is —(CH₂)₃—. In one embodiment, the bridge is —CH₂—O—CH₂—. A “spiro” bicyclic ring system shares a single ring atom (usually a quaternary carbon atom) between two rings.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracyclic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O—(alkyl), wherein alkyl is defined above.

An “alkoxyalkyl” group is -(alkyl)-O—(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O))(R^{#}) or —N(alkyl)C(O)(R^{#}), wherein each alkyl and R^{#} are independently as defined above.

A “sulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R*), —N(alkyl)C(O)NH₂, —NHC(O)N(R^{#})₂, —NHC(O)NH(R^{#}), or —NH(CO)NHR^{#}, wherein each alkyl and Rare independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stercomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See. e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, I N, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

As used herein and unless otherwise indicated, “atropisomers” refer to stereoisomers resulting from hindered rotation about a single bond axis where the rotational barrier is high enough to allow for the isolation of the individual rotational isomers

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

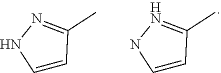

As readily understood by one skilled in the art, a wide variety of functional groups and other stuctures may exhibit tautomerism and all tautomers of compounds of formula (I) are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In some embodiments, “treating” means an alleviation, in whole or in part, of a disorder, disease or condition, or a slowing, or halting of further progression or worsening of those symptoms. In another embodiment, “treating” means and alleviation, in whole or in part, of a disorder, disease or condition, or symptoms associated with a condition, wherein the condition is treatable or preventable by inhibition of KRAS; preferably G12D and/or G12V.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the condition is a condition, treatable or preventable by inhibition of KRAS; preferably G12D and/or G12V.

The term “effective amount” in connection with a compound means an amount capable of treating or preventing a disorder, disease or condition, or symptoms thereof, disclosed herein.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

### COMPOUNDS

Aspect 1: Provided herein are compounds having the following formula (I):

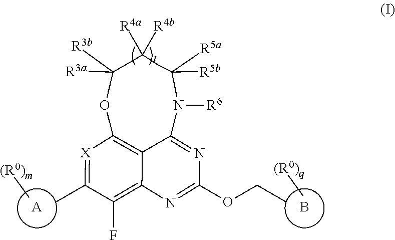

and pharmaceutically acceptable salts, tautomers, stereoisomers, enantiomers, atropisomers, isotopologues, and prodrugs thereof, wherein:
ring A is unsubstituted or substituted aryl, or unsubstituted or substituted heteroaryl;
ring B is unsubstituted or substituted cycloalkyl, or unsubstituted or substituted heterocyclyl;
X is N, or C—R⁸;
   each of R⁰ is, independently, H, halogen, amino, —CN, —OH, unsubstituted or substituted C₁₋₄ alkyl, unsubstituted or substituted C₁₋₄ alkenyl, unsubstituted or substituted C₁₋₄ alkynyl, unsubstituted or substituted C₁₋₄ alkoxy, unsubstituted or substituted C₃₋₅ cycloalkyl, unsubstituted or substituted 3-member to 5-member heterocyclyl, unsubstituted or substituted C₁₋₄ alkylamino, carboxy, nitro, thiol, or thioether; or one or more pairs of the R⁰ groups, together with the atom to which they are attached, form unsubstituted or substituted cycloalkyl, unsubstituted or substituted heterocyclyl, unsubstituted or substituted aryl, or unsubstituted or substituted heteroaryl;
optionally, each of R^{3a}, R^{3b}, R^{4a}, R^{4b}, R^{5a}, and R^{5b}, is, independently, H, halogen, unsubstituted or substituted amino, —CN, —OH, unsubstituted or substituted C₁₋₄ alkyl, unsubstituted or substituted C₁₋₄ alkoxy, unsubstituted or substituted C₃₋₅ cycloalkyl, unsubstituted or substituted 3-member to 5-member heterocyclyl, unsubstituted or substituted C₁₋₄ alkylamino, carboxy, nitro, thiol, or thioether; or
the R^{3a}, and R^{3b} groups, together with the atom to which they are attached, form unsubstituted or substituted cycloalkyl, or unsubstituted or substituted heterocyclyl; or
the R^{4a}, and R^{4b} groups, together with the atom to which they are attached, form unsubstituted or substituted cycloalkyl, or unsubstituted or substituted heterocyclyl; or
the R^{5a}, and R^{5b} groups, together with the atom to which they are attached, form unsubstituted or substituted cycloalkyl, or unsubstituted or substituted heterocyclyl; or
the R^{3a}, and R^{4a} groups, together with the atoms to which they are attached, form unsubstituted or substituted cycloalkyl, or unsubstituted or substituted heterocyclyl; or
the R^{3a}, and R^{5a} groups, together with the atoms to which they are attached, form unsubstituted or substituted cycloalkyl, or unsubstituted or substituted heterocyclyl; or
the R^{4a}, and R^{5a} groups, together with the atoms to which they are attached, form unsubstituted or substituted cycloalkyl, or unsubstituted or substituted heterocyclyl; or
R⁶ is H, unsubstituted or substituted C₁₋₈ alkyl, unsubstituted or substituted C₁₋₈ alkoxy, unsubstituted or substituted C₃₋₈cycloalkyl, or unsubstituted or substituted 3-member to 8-member heterocyclyl;

R⁸ is H, halogen, unsubstituted or substituted C₁₋₄ alkyl, unsubstituted or substituted C₁₋₄ alkenyl, unsubstituted or substituted C₃₋₅ cycloalkyl, unsubstituted or substituted C₁₋₄ alkoxyl, unsubstituted or substituted C₁₋₄ halogenated alkyl, unsubstituted or substituted C₃₋₅ halogenated cycloalkyl, unsubstituted or substituted C₁₋₄ halogenated alkoxyl, CN, OH, or amino;
t is 0, or 1; and
each of m, and q is, independently, an integer between 0 and the maximum number of the substituent groups allowed on rings A, and B, respectively.

Aspect 2A: Provided herein are compounds having the following formula (I):

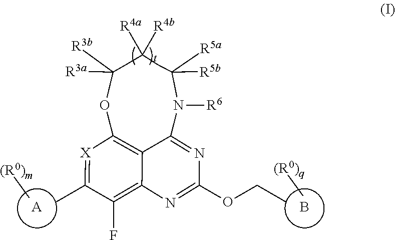

and pharmaceutically acceptable salts, tautomers, stereoisomers, enantiomers, atropisomers, isotopologues, and prodrugs thereof, wherein:
ring A is unsubstituted or substituted aryl, or unsubstituted or substituted heteroaryl;
ring B is unsubstituted or substituted cycloalkyl, or unsubstituted or substituted heterocyclyl;
X is N, or C—R⁸;
each of R⁰ is, independently, H, halogen, amino, —CN, —OH, unsubstituted or substituted C₁₋₄-alkyl, unsubstituted or substituted C₁₋₄ alkenyl, unsubstituted or substituted C₁₋₄ alkynyl, unsubstituted or substituted C₁₋₄ alkoxy, unsubstituted or substituted C₃₋₅ cycloalkyl, unsubstituted or substituted 3-member to 5-member heterocyclyl, unsubstituted or substituted C₁₋₄ alkylamino, carboxy, nitro, thiol, or thioether; or one or more pairs of the R⁰ groups, together with the atom to which they are attached, form unsubstituted or substituted cycloalkyl, unsubstituted or substituted heterocyclyl, unsubstituted or substituted aryl, or unsubstituted or substituted heteroaryl;
the R^{3a}, and R^{4a} groups, together with the atoms to which they are attached, form unsubstituted or substituted cycloalkyl, or unsubstituted or substituted heterocyclyl; and each of R^{3b}, R^{4b}, R^{5a}, and R^{5b}, is, independently, H, halogen, unsubstituted or substituted amino, —CN, —OH, unsubstituted or substituted C₁₋₄ alkyl, unsubstituted or substituted C₁₋₄ alkoxy, unsubstituted or substituted C₃₋₅-cycloalkyl, unsubstituted or substituted 3-member to 5-member heterocyclyl, unsubstituted or substituted C₁₋₄ alkylamino, carboxy, nitro, thiol, or thioether; or the R^{5a}, and R^{5a} b groups, together with the atom to which they are attached, form unsubstituted or substituted cycloalkyl, or unsubstituted or substituted heterocyclyl; or
the R^{3a}, and R^{5a} groups, together with the atoms to which they are attached, form unsubstituted or substituted cycloalkyl, or unsubstituted or substituted heterocyclyl; and each of R^{3a}, R^{3b}, R^{4a}, R^{4b}, and R^{5b}, is, independently, H, halogen, unsubstituted or substituted amino, —CN, —OH, unsubstituted or substituted C₁₋₄ alkyl, unsubstituted or substituted C₁₋₄ alkoxy, unsubstituted or substituted C₃₋₅cycloalkyl, unsubstituted or substituted 3-member to 5-member heterocyclyl, unsubstituted or substituted C₁₋₄ alkylamino, carboxy, nitro, thiol, or thioether; or the R^{4a}, and R^{4b} groups, together with the atom to which they are attached, form unsubstituted or substituted cycloalkyl, or unsubstituted or substituted heterocyclyl; or the R^{4a}, and R^{5a} groups, together with the atoms to which they are attached, form unsubstituted or substituted cycloalkyl, or unsubstituted or substituted heterocyclyl; and each of R^{3a}, R^{3b}, R^{4b}, and R^{5b}, is, independently, H, halogen, unsubstituted or substituted amino, —CN, —OH, unsubstituted or substituted C₁₋₄-alkyl, unsubstituted or substituted C₁₋₄ alkoxy, unsubstituted or substituted C₃₋₅ cycloalkyl, unsubstituted or substituted 3-member to 5-member heterocyclyl, unsubstituted or substituted C₁₋₄ alkylamino, carboxy, nitro, thiol, or thioether; or the R^{3a}, and R^{3b} groups, together with the atom to which they are attached, form unsubstituted or substituted cycloalkyl, or unsubstituted or substituted heterocyclyl;
R⁶ is H, unsubstituted or substituted C₁₋₈ alkyl, unsubstituted or substituted C₁₋₈ alkoxy, unsubstituted or substituted C₃₋₈ cycloalkyl, or unsubstituted or substituted 3-member to 8-member heterocyclyl:

R⁸ is H, halogen, unsubstituted or substituted C₁₋₄ alkyl, unsubstituted or substituted C₁₋₄ alkenyl, unsubstituted or substituted C₃₋₅ cycloalkyl, unsubstituted or substituted C₁₋₄ alkoxyl, unsubstituted or substituted C₁₋₄ halogenated alkyl, unsubstituted or substituted C₃₋₅ halogenated cycloalkyl, unsubstituted or substituted C₁₋₄ halogenated alkoxyl, CN, OH, or amino;
t is 0, or 1; and
each of m, and q is, independently, an integer between 0 and the maximum number of the substituent groups allowed on rings A, and B, respectively.

In some embodiment, m is an integer between 0 and 5. In some embodiment, m is an integer between 1 and 4. In some embodiment, m is an integer between 2 and 3. In some embodiment, m is an integer of 2 or 3. In some embodiment, q is an integer between 0 and 5. In some embodiment, q is an integer between 1 and 4. In some embodiment, q is an integer between 1 and 3. In some embodiment, q is an integer of 1 or 2.

In one embodiment, X is N. In one embodiment, X is C—R⁸. In one embodiment, X is C—H, C—F, C—Cl, or C—CF₃. In one embodiment, X is C—Cl. In one embodiment, X is C—CF₃.

In one embodiment, R⁶ is unsubstituted or substituted C₁₋₈ alkyl, unsubstituted or substituted heteroalkyl, unsubstituted or substituted C₃₋₈ cycloalkyl, unsubstituted or substituted 3-member to 8-member heterocyclyl. In one embodiment, R⁶ is methyl.

### Aspect 3:

In one embodiment, ring A is an aryl group (e.g., phenyl or naphthyl) optionally substituted with one or more substituents. In one embodiment, the substituent is F, Cl, Br, amino, —CN, OH, —CF₃, —CHF₂, —CH₂F, —CF₂CH₃, —CF₂CF₃, —OCHF₂, —OCF₃, vinyl(—CH═CH),-propylenyl (e.g., —C(CH₃)═CH), —CF—CH, aryl, heteroaryl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy. In one embodiment, the substituent is F, Cl, Br, amino, —CN, OH, —CF₃, —CHF₂, —CH₂F, —CF₂CH₃, —CF₂CF₃, —OCHF₂, —OCF₃, vinyl(—CH═CH),-propylenyl(such as —C(CH₃)—CH), —CF—CH, aryl, heteroaryl, ethynyl, propynyl, butynyl, pentynyl, hexynyl, cyclopropyl, methylcyclopropyl, fluorocyclopropyl, difluorocyclopropyl, fluoromethylcyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy.

In one embodiment, ring A is a 5- to 7-membered monocyclic heteroaryl or 8- to 12-membered bicyclic heteroaryl group optionally substituted with one or more substituents. In some preferred embodiments, ring A is pyridyl, benzothiazolyl, quinolinyl, isoquinolinyl, pyrazolopyridinyl, benzoimidazolyl, quinazolinyl, or quinazolinyl. In one embodiment, the substituent is F, Cl, Br, C(CH₃)—CH or —CH—CH, —CF—CH, —CN, OH, —NH₂, —CF₃, —CHF₂, —CH₂F, —CF₂CH₃, —CF₂CF₃, —OCHF₂, —OCF₃, aryl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy. In one embodiment, the substituent is F, Cl, Br, C(CH₃)═CH or —CH═CH, —CF═CH, —CN, OH, NH₂, —CF₃, —CHF₂, —CH₂F, —CF₂CH₃, —CF₂CF₃, —OCHF₂, —OCF₃, aryl, ethynyl, propynyl, butynyl, pentynyl, hexynyl, cyclopropyl, methylcyclopropyl, fluorocyclopropyl, difluorocyclopropyl, fluoromethylcyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy. In some preferred embodiments, ring A is pyridyl, benzothiazolyl, quinolinyl, isoquinolinyl, pyrazolopyridinyl, benzoimidazolyl, quinazolinyl, or quinazolinyl.

In one embodiment, ring A is

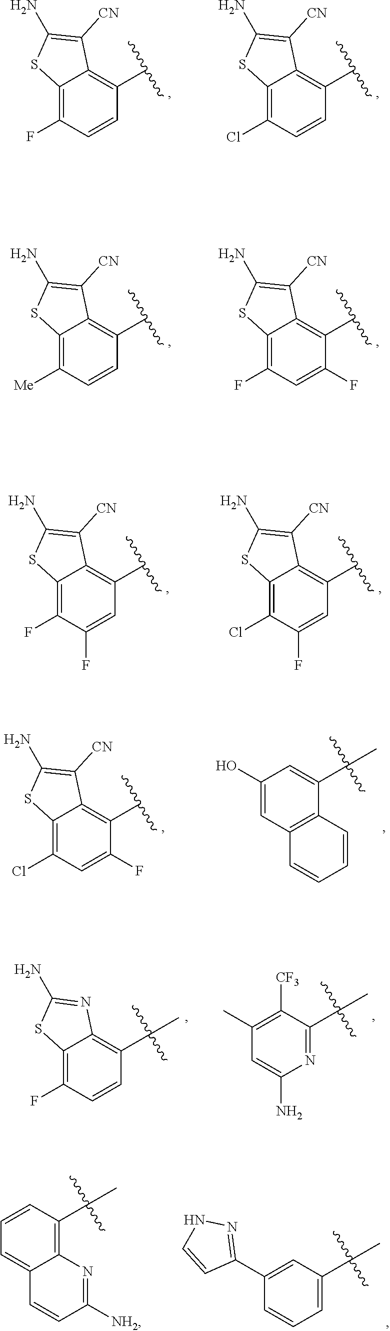

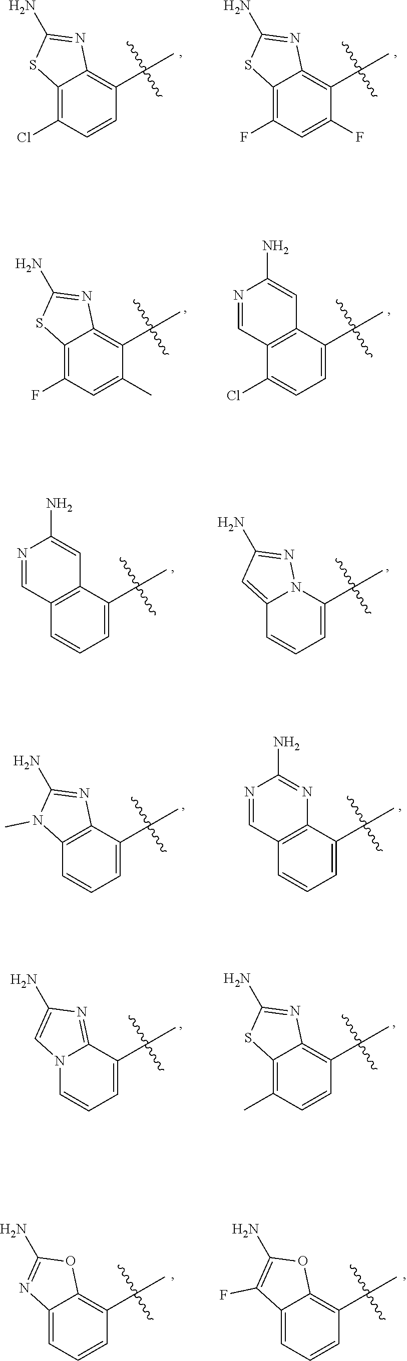

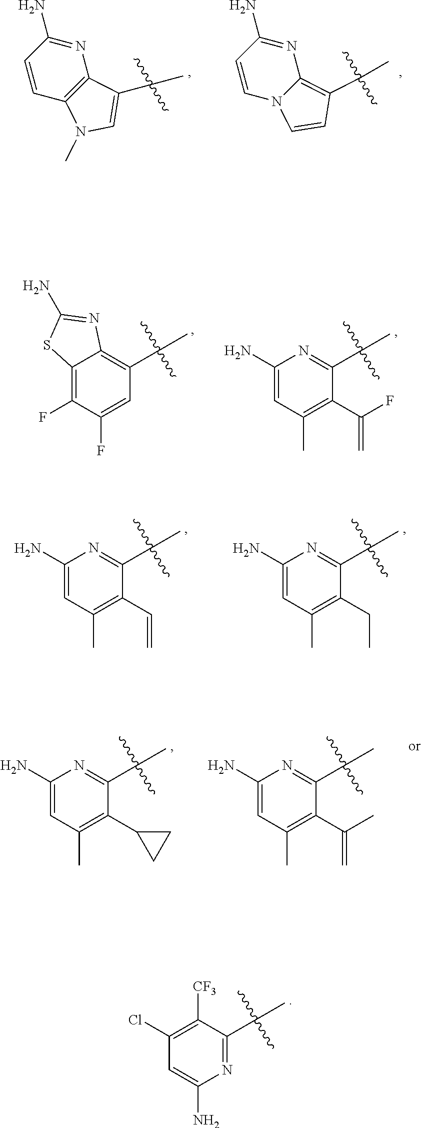

In one embodiment, ring A is

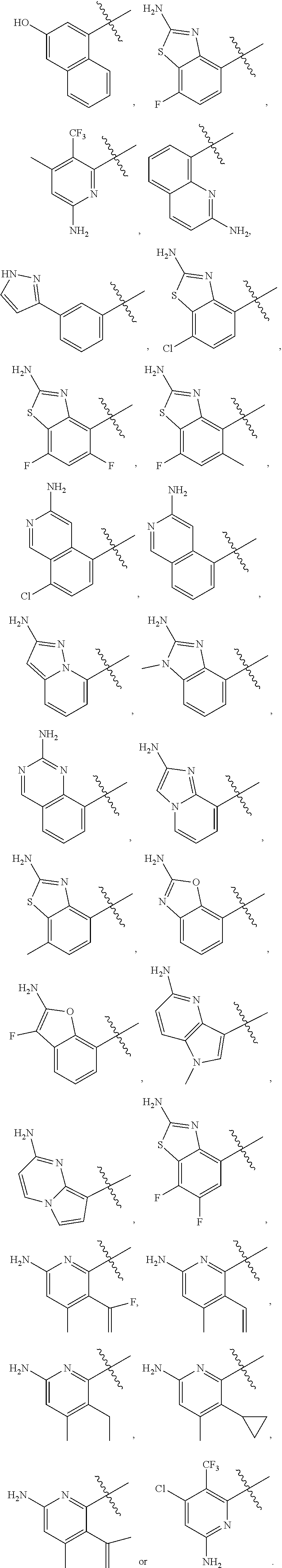

In one embodiment, ring A is

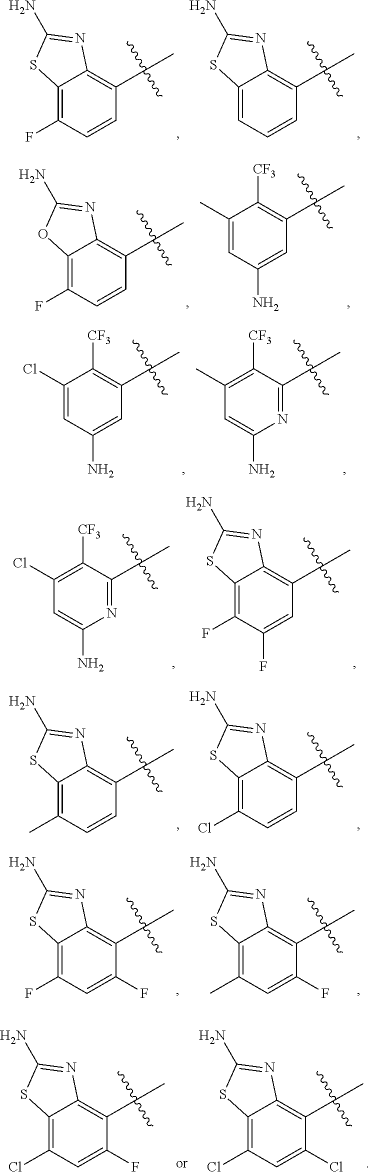

In one embodiment, ring A is

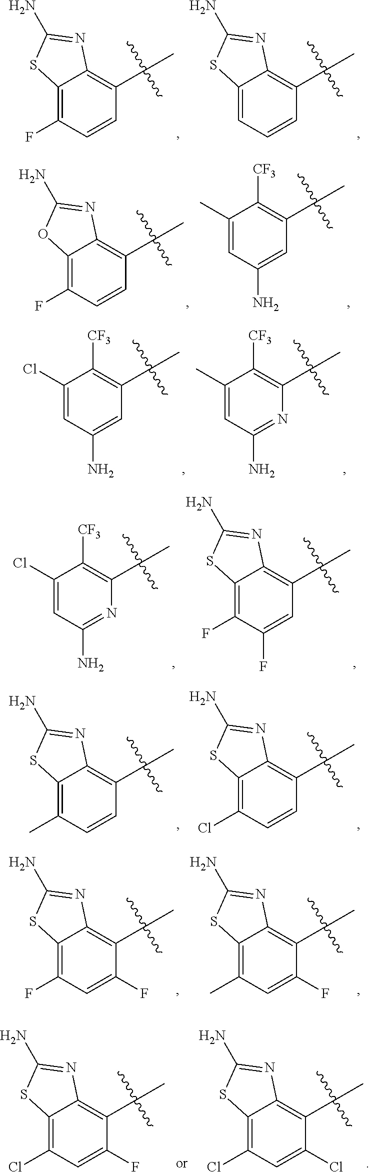

In one embodiment, ring A is

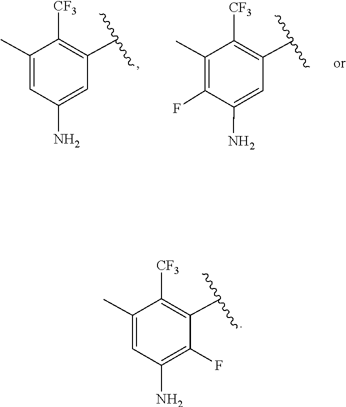 [SMILES: *c1cc(N)cc(C)c1C(F)(F)F ; *c1cc(N)c(F)c(C)c1C(F)(F)F ; *c1c(F)c(N)cc(C)c1C(F)(F)F]

In one embodiment, ring A is

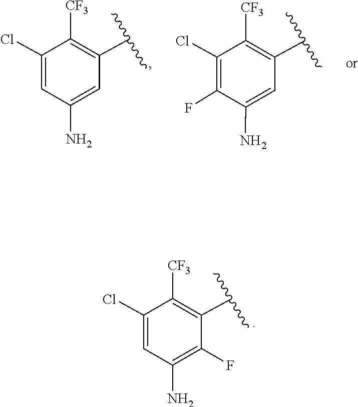 [SMILES: *c1cc(N)cc(Cl)c1C(F)(F)F ; *c1cc(N)c(F)c(Cl)c1C(F)(F)F ; *c1c(F)c(N)cc(Cl)c1C(F)(F)F]

In one embodiment, ring A is

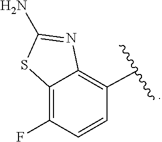 [SMILES: *c1ccc(F)c2sc(N)nc12]

In one embodiment, ring A is

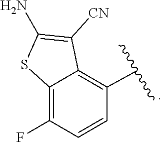 [SMILES: *c1ccc(F)c2sc(N)c(C#N)c12]

### Aspect 4:

In one embodiment, ring B is unsubstituted or substituted cycloalkyl or unsubstituted or substituted heterocyclyl. In one embodiment, the heterocyclyl comprises at least one oxygen as the ring member. In one embodiment, the heterocyclyl comprises at least one nitrogen as the ring member.

In one embodiment, ring B is cyclopropyl, cyclobutyl, cyclopentyl, cyclohexyl, cycloheptyl, cyclooctyl, 3- to 9-membered heterocylic ring comprising one or two or three nitrogen atoms as the ring members. In one embodiment, ring B is oxazolidinyl, imidazolidinyl, thiazolidinyl, pyrazolidinyl, morpholinyl, thiomorpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl, tetrahydropyridinyl, azetidinyl, pyrrolidinyl, octahydroindolizinyl, octahydroquinolizinyl, hexahydro-1H-pyrrolizinyl, tetrahydroisoquinolinyl, or tetrahydropyridyl; and ring B is optionally substituted. In one embodiment, ring B is oxetanyl, tetrahydrofuryl, tetrahydro-2H-pyranyl, dihydro-2H-pyranyl, oxabicyclo[2,1,1]hexyl, oxabicyclo[2,2,1]heptyl, oxaspiro[3.3]heptyl, oxabicyclo[3.2.1]octyl, oxabicyclo[2,2,2]octyl, oxaspiro[3.5]nonyl, or oxaspiro[3.4]octyl; and ring B is optionally substituted. In one embodiment, ring B is optionally substituted with halogen, cyano, hydroxy, alkoxy, or alkyl optionally substituted with halogen, cyano, hydroxy, alkoxy, heterocyclyl, cycloalkyl or cycloalkyloxy.

In one embodiment, ring B is cyclopropyl, cyclobutyl, cyclopentyl, cyclohexyl, cycloheptyl, cyclooctyl, 3- to 9-membered heterocylic ring comprising one or two or three nitrogen atoms as the ring members, oxazolidinyl, imidazolidinyl, thiazolidinyl, pyrazolidinyl, morpholinyl, thiomorpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl or tetrahydropyridinyl optionally substituted with one or more substituents. In one embodiment, the substituent is methyl, ethyl, propyl, butyl, pentyl, hexyl, heptyl, —C₂₋₈-alkenyl, —C₂₋₈-alkynyl, C₁₋₈ alkoxy-C₁₋₈ alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or oxo; or two substituents together with the carbon atoms to which they are attached, form a 3- to 8-membered unsaturated or saturated ring, said ring comprising 0, 1, 2, or 3 heteroatoms independently selected from nitrogen, oxygen or sulfur.

In one embodiment, ring B is

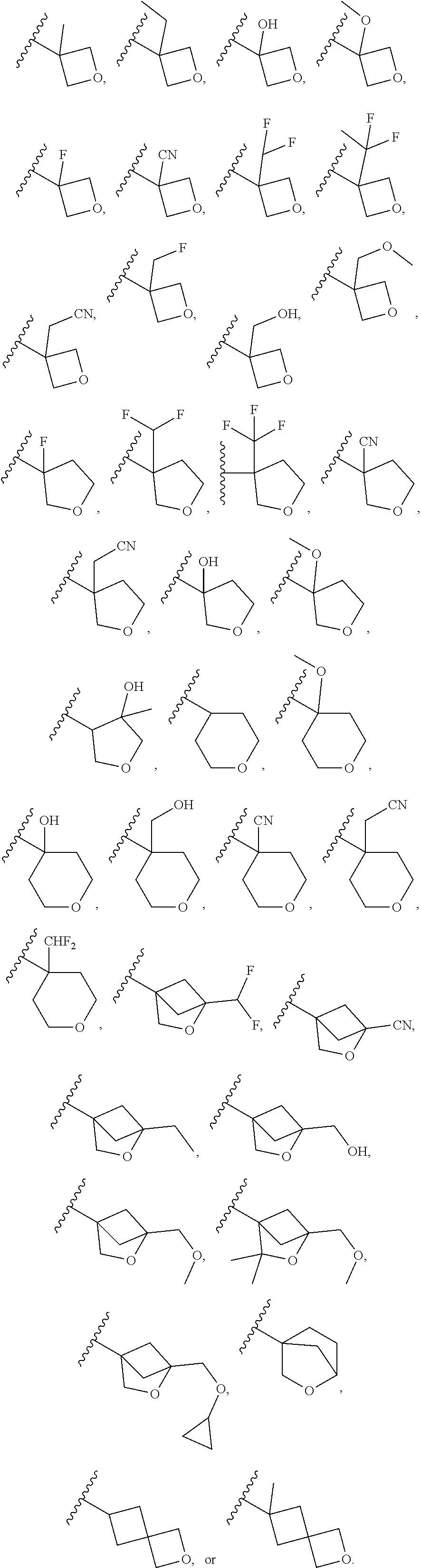

In one embodiment, ring B is azetidyl, pyridyl, isoxazolyl, oxazolyl, dihydro-2H-pyranyl, tetrahydro-2H-pyranyl, pyrrolidinonyl, azaspiro[3.3]heptyl, azabicyclo[2,1,1]hexyl, pyrrolidyl, 1H-pyrazolyl; and ring B is optionally substituted.

In one embodiment, ring B is optionally substituted with halogen, cyano, hydroxy, alkoxy, or alkyl optionally substituted with halogen, cyano, hydroxy, or alkoxy.

In one embodiment, ring B is

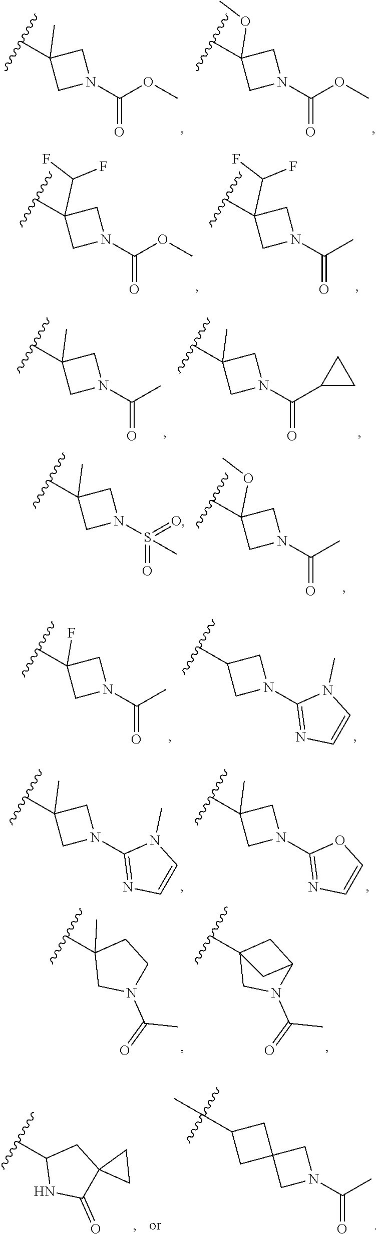

In one embodiment, ring B is azetidyl, pyridyl, isoxazolyl, oxazolyl, dihydro-2H-pyranyl, tetrahydro-2H-pyranyl, pyrrolidinonyl, azaspiro[3.3]heptyl, azabicyclo[2,1,1]hexyl, pyrrolidyl, 1H-pyrazolyl; and ring B is optionally substituted.

In one embodiment, ring B is cyclopropyl, cyclobutyl, cyclopentyl, cyclohexyl, cycloheptyl, or azetidyl; and ring B is optionally substituted.

In one embodiment, ring B is

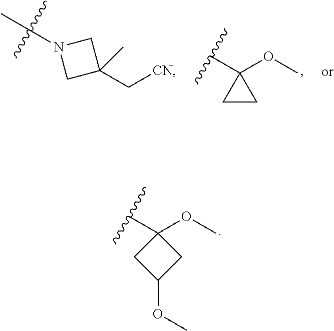

In one embodiment, ring B is

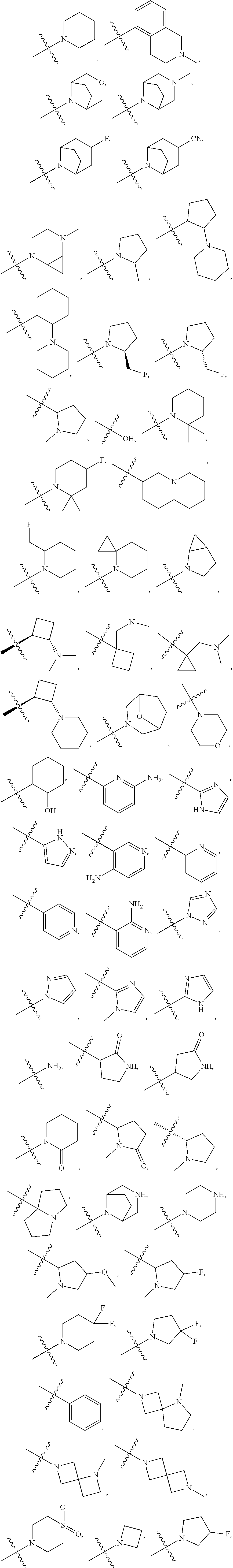

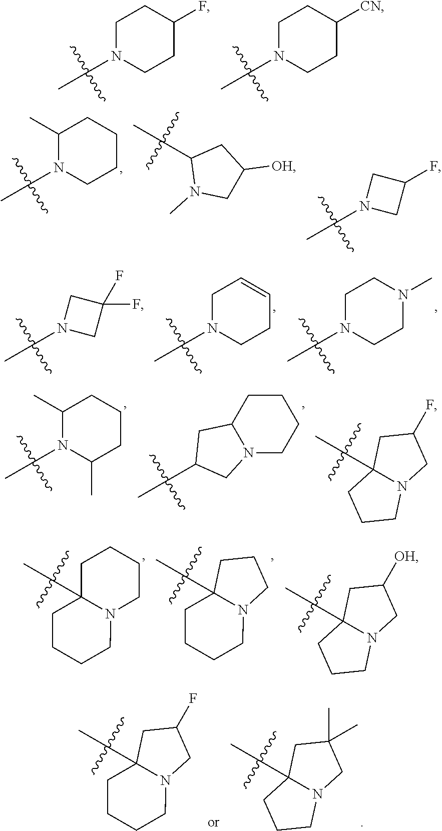

In one embodiment, ring B is

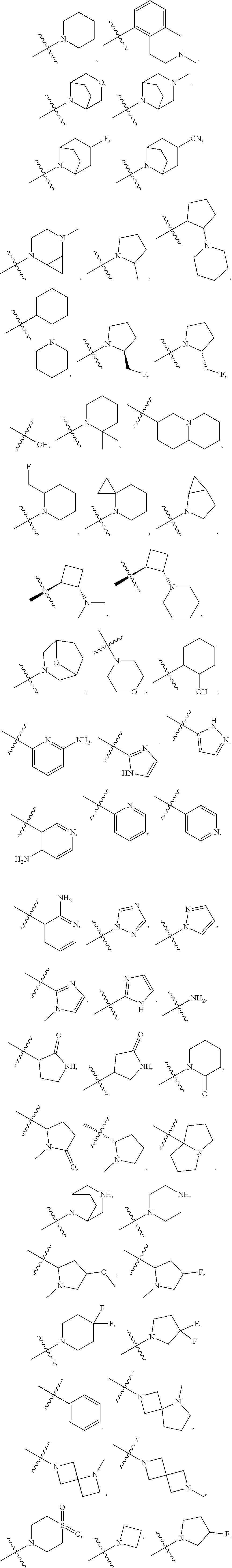

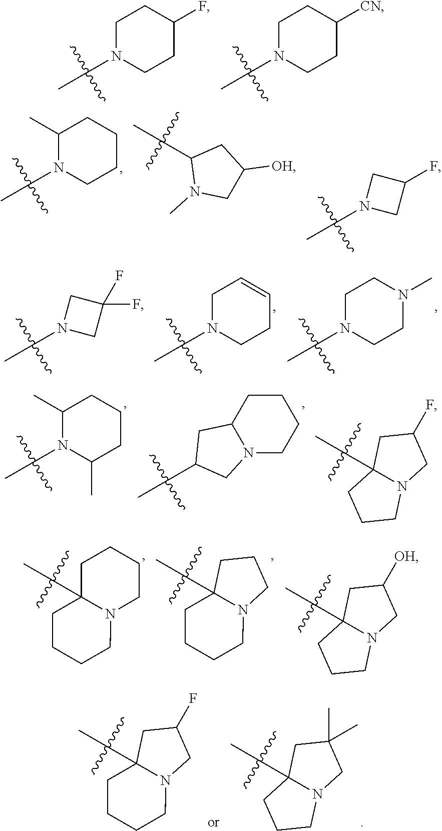

In one embodiment, ring B is

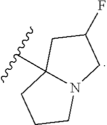 [SMILES: *C12CCCN1CC(F)C2]

In one embodiment, ring B is

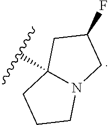 [SMILES: *[C@@]12CCCN1C[C@H](F)C2]

In one embodiment, ring B is

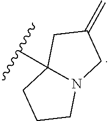 [SMILES: *C12CCCN1CC(=C)C2]

In one embodiment, ring B is

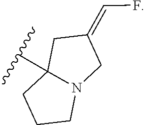 [SMILES: *C12CCCN1C/C(=C\F)C2]

In one embodiment, ring B is

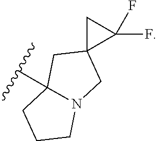 [SMILES: *C12CCCN1CC1(C2)CC1(F)F]

In one embodiment, ring B is

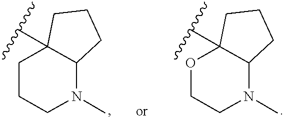 [SMILES: *C12CCCC1N(C)CCC2 ; *C12CCCC1N(C)CCO2]

In one embodiment, ring B is

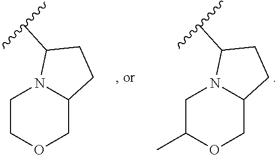 [SMILES: *C1CCC2COCCN12 ; *C1CCC2COC(C)CN12]

In one embodiment, ring B is

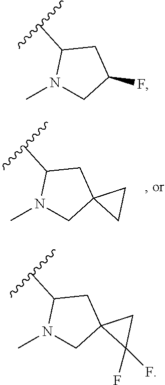

In one embodiment, ring B is

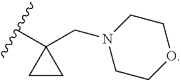 [SMILES: *C1(CN2CCOCC2)CC1]

In one embodiment, ring B is

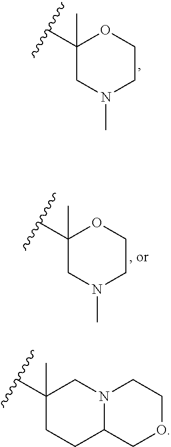

In one embodiment, ring B is

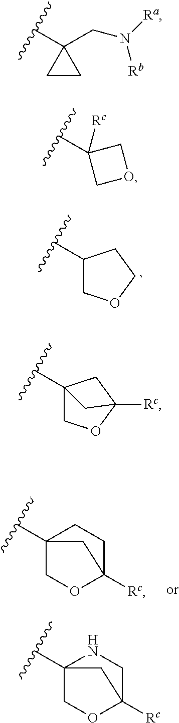

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl. In one embodiment, ring B is

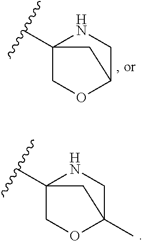

### Aspect 5:

In one embodiment, ring A is substituted or unsubstituted phenyl, substituted or unsubstituted naphthyl, substituted or unsubstituted benzo[b]thiophenyl, or substituted or unsubstituted benzo[d]thiazolyl.

In one embodiment, ring A is

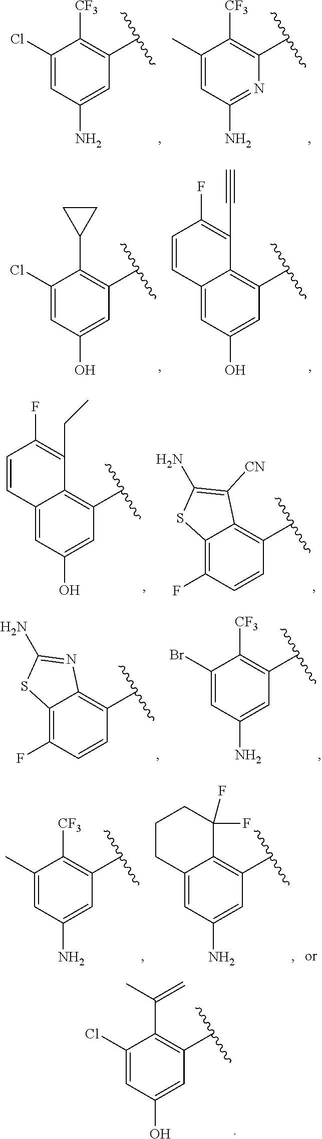

In one embodiment, ring A is

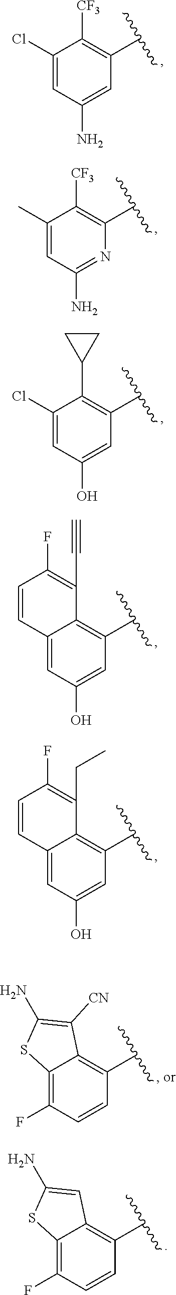

In one embodiment, ring A is

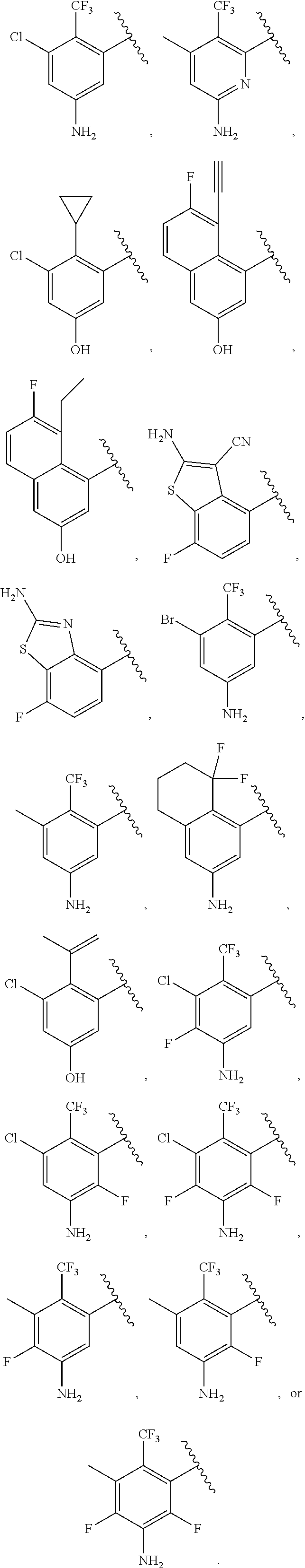

In one embodiment, ring A is

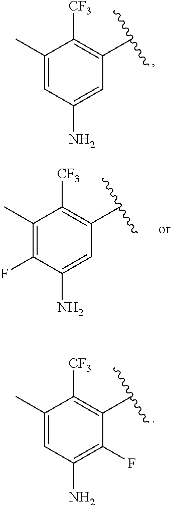

In one embodiment, ring A is

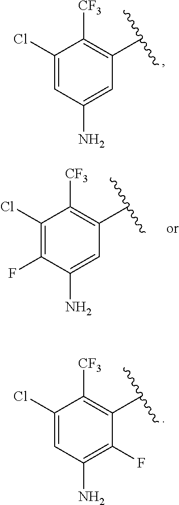

### Aspect 6:

In one embodiment, ring B is substituted or unsubstituted hexahydro-1H-pyrrolizinyl, substituted or unsubstituted pyrrolidinyl, substituted or unsubstituted aminomethylcyclopropyl, substituted or unsubstituted oxetanyl, substituted or unsubstituted tetrahydrofuranyl, substituted or unsubstituted oxabicyclo[2,1,1]hexanyl, substituted or unsubstituted oxabicyclo[2,2,1]heptanyl.

In one embodiment, ring B is

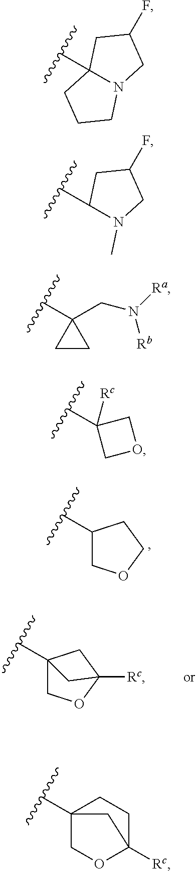

wherein each of R^{a} and R^{b} is, independently, substituted or unsubstituted C₁₋₄ alkyl;

substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl.

In one embodiment, ring B is

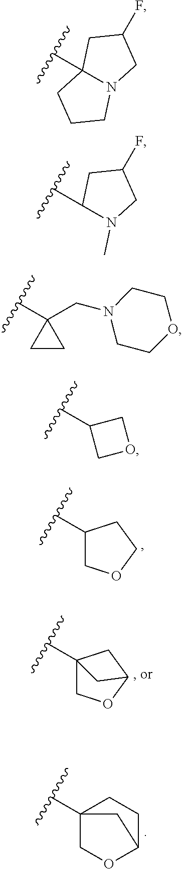

In one embodiment, ring B is

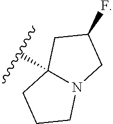 [SMILES: *[C@@]12CCCN1C[C@H](F)C2]

In one embodiment, ring B is

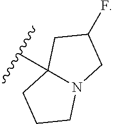 [SMILES: *C12CCCN1CC(F)C2]

In one embodiment, ring B is

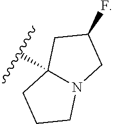 [SMILES: *[C@@]12CCCN1C[C@H](F)C2]

In one embodiment, ring B is

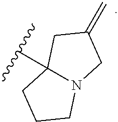 [SMILES: *C12CCCN1CC(=C)C2]

In one embodiment, ring B is

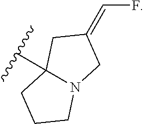 [SMILES: *C12CCCN1C/C(=C\F)C2]

In one embodiment, ring B is

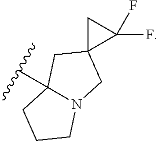 [SMILES: *C12CCCN1CC1(C2)CC1(F)F]

In one embodiment, ring B is

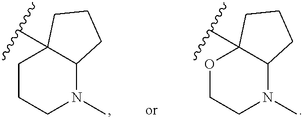 [SMILES: *C12CCCC1N(C)CCC2 ; *C12CCCC1N(C)CCO2]

In one embodiment, ring B is

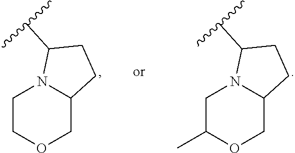 [SMILES: *C1CCC2COCCN12 ; *C1CCC2COC(C)CN12]

In one embodiment, ring B is In one

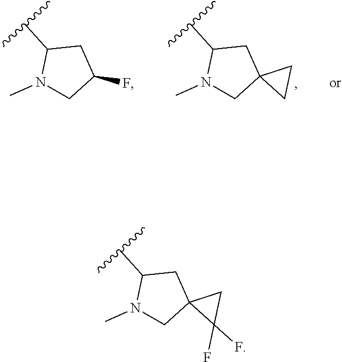

In one embodiment, ring B is

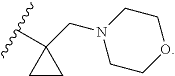 [SMILES: *C1(CN2CCOCC2)CC1]

In one embodiment, ring B is

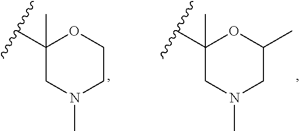

or

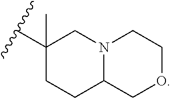 [SMILES: *C1(C)CCC2COCCN2C1]

In one embodiment, ring B is

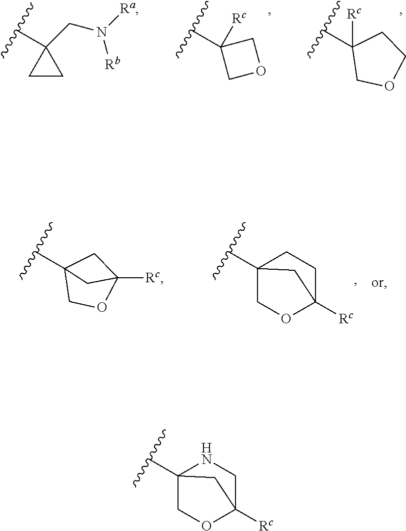

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl. In one embodiment, ring B is

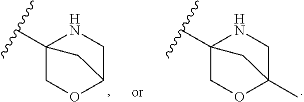

### Aspect 7: In One Embodiment, t is 0.

### Group 2.1: In One Embodiment, X is N.

In one embodiment, ring A is

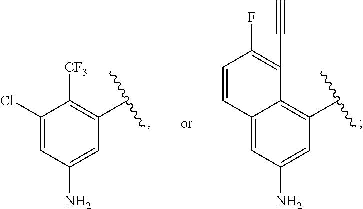 [SMILES: *c1cc(N)cc(Cl)c1C(F)(F)F ; *c1cc(N)cc2ccc(F)c(C#C)c12]

and ring B is

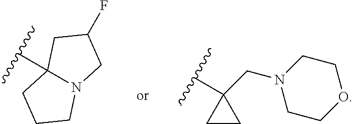 [SMILES: *C12CCCN1CC(F)C2 ; "or" ; *C1(CN2CCOCC2)CC1]

In one embodiment, R¹ is methyl, or Cl. In one embodiment, R² is —CF₃. In one embodiment, R⁷ is —NH₂.

Group 2,1,1: In one embodiment, ring A is

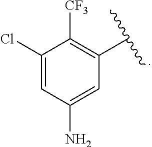 [SMILES: *c1cc(N)cc(Cl)c1C(F)(F)F]

In one embodiment, ring B is

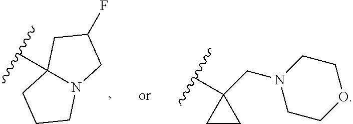 [SMILES: *C12CCCN1CC(F)C2 ; "," ; "or" ; *C1(CN2CCOCC2)CC1]

Group 2,1,1,1: In one embodiment, ring B is

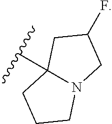 [SMILES: *C12CCCN1CC(F)C2]

In one embodiment, ring B is

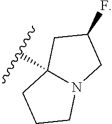 [SMILES: *[C@@]12CCCN1C[C@H](F)C2]

In one embodiment, R⁶ is unsubstituted or substituted C₁₋₅alkyl, unsubstituted or substituted C₃₋₅ cycloalkyl, or unsubstituted or substituted 3-member to 5-member heterocyclyl.

In one embodiment, R⁶ is methyl, ethyl, cyclopropyl, or cyclobutyl, optionally substituted by CN or OH. In one embodiment, R⁶ is methyl. In one embodiment, R⁶ is 2-hydroxylethyl. In one embodiment, R⁶ is cyclopropyl.

In one embodiment, each of R^{3a}, R^{3b}, R^{5a}, and R^{5b}, independently, is H, OH, CN, amino, or unsubstituted or substituted C₁₋₄ alkyl. In one embodiment, each of R^{3a}, R^{3b}, R^{5a}, and R^{5b}, independently, is H, or unsubstituted or substituted C₁₋₄ alkyl. In one embodiment, each of R^{3a}, R^{3b}, R^{5a}, and R^{5b}, independently, is H, unsubstituted or substituted methyl, or unsubstituted or substituted ethyl. In one embodiment, each of R^{3a}, R^{3b}, R^{5a}, and R^{5b}, independently, is H, methyl, or ethyl, optionally substituted by OH, CN, amino, or methylamino.

In one embodiment, the compound is

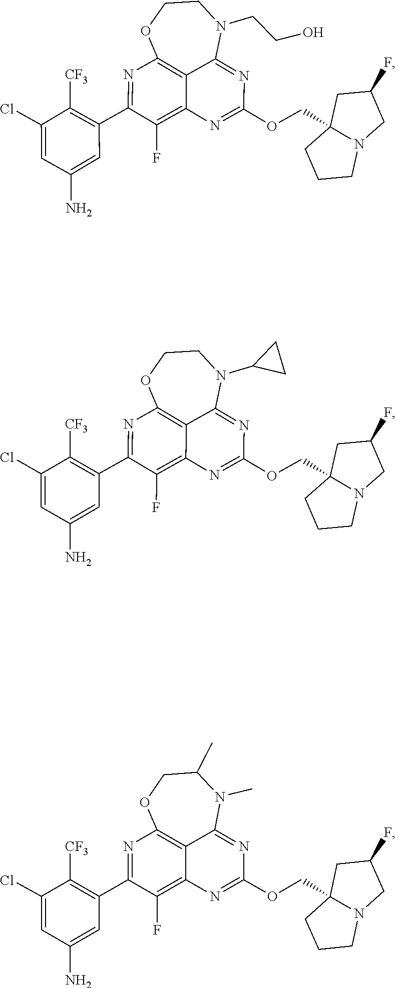 [SMILES: Nc1cc(Cl)c(C(F)(F)F)c(-c2nc3c4c(nc(OC[C@@]56CCCN5C[C@H](F)C6)nc4c2F)N(CCO)CCO3)c1 ; Nc1cc(Cl)c(C(F)(F)F)c(-c2nc3c4c(nc(OC[C@@]56CCCN5C[C@H](F)C6)nc4c2F)N(C2CC2)CCO3)c1 ; CC1COc2nc(-c3cc(N)cc(Cl)c3C(F)(F)F)c(F)c3nc(OC[C@@]45CCCN4C[C@H](F)C5)nc(c23)N1C]

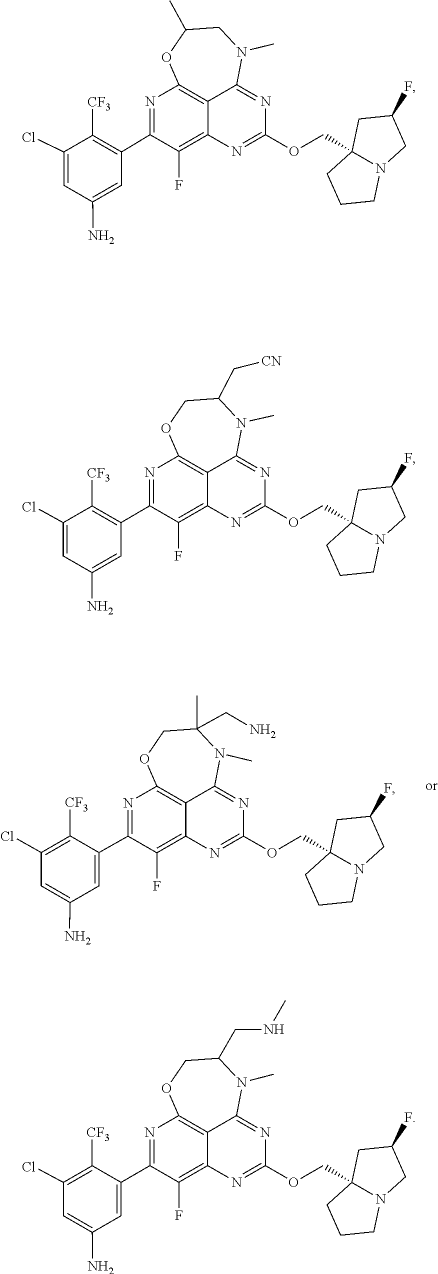 [SMILES: CC1CN(C)c2nc(OC[C@@]34CCCN3C[C@H](F)C4)nc3c(F)c(-c4cc(N)cc(Cl)c4C(F)(F)F)nc(c23)O1 ; CN1c2nc(OC[C@@]34CCCN3C[C@H](F)C4)nc3c(F)c(-c4cc(N)cc(Cl)c4C(F)(F)F)nc(c23)OCC1CC#N ; CN1c2nc(OC[C@@]34CCCN3C[C@H](F)C4)nc3c(F)c(-c4cc(N)cc(Cl)c4C(F)(F)F)nc(c23)OCC1(C)CN ; CNCC1COc2nc(-c3cc(N)cc(Cl)c3C(F)(F)F)c(F)c3nc(OC[C@@]45CCCN4C[C@H](F)C5)nc(c23)N1C]

In one embodiment, R⁶ is methyl.

In one embodiment, R^{5a} and R^{5b}, together with the atom to which they are attached, form unsubstituted or substituted C₃₋₅cycloalkyl. In one embodiment, R^{5a} and R^{5b}, together with the atom to which they are attached, form unsubstituted or substituted cyclopropyl, or unsubstituted or substituted cyclobutyl.

In one embodiment, the compound is

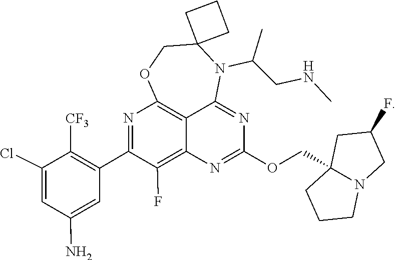 [SMILES: CNCC(C)N1c2nc(OC[C@@]34CCCN3C[C@H](F)C4)nc3c(F)c(-c4cc(N)cc(Cl)c4C(F)(F)F)nc(c23)OCC12CCC2]

In one embodiment, R^{5a} and R^{5b}, together with the atom to which they are attached, form unsubstituted or substituted heterocyclyl. In one embodiment, the heterocyclyl is oxetanyl, tetrahydrofuranyl, tetrahydropyranyl, or dioxanyl. In one embodiment, the heterocyclyl is oxetanyl, or tetrahydrofuranyl. In one embodiment, the heterocyclyl is oxetanyl. In one embodiment, the heterocyclyl is tetrahydrofuranyl.

In one embodiment, the compound is

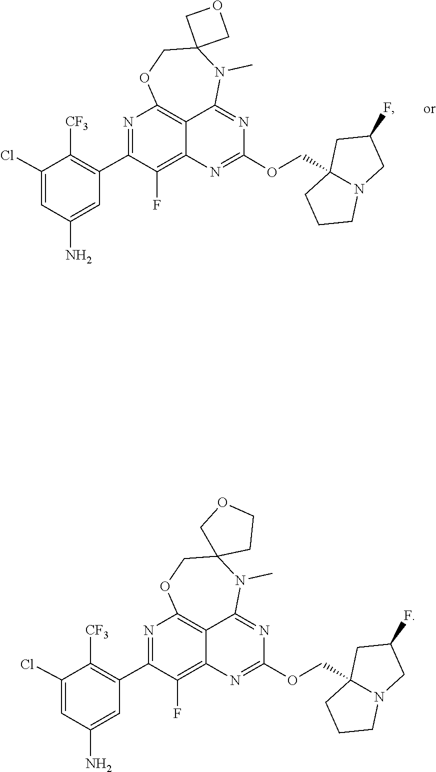 [SMILES: CN1c2nc(OC[C@@]34CCCN3C[C@H](F)C4)nc3c(F)c(-c4cc(N)cc(Cl)c4C(F)(F)F)nc(c23)OCC12COC2 ; CN1c2nc(OC[C@@]34CCCN3C[C@H](F)C4)nc3c(F)c(-c4cc(N)cc(Cl)c4C(F)(F)F)nc(c23)OCC12CCOC2]

Group 2.1.2: In one embodiment, ring A is

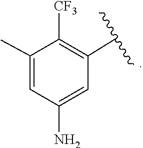 [SMILES: *c1cc(N)cc(C)c1C(F)(F)F]

In one embodiment, ring B is

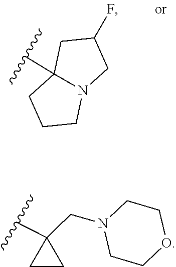 [SMILES: *C12CCCN1CC(F)C2 ; *C1(CN2CCOCC2)CC1]

Group 2.1.2.1: In one embodiment, ring B is

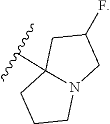 [SMILES: *C12CCCN1CC(F)C2]

In one embodiment, ring B is

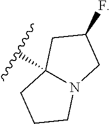 [SMILES: *[C@@]12CCCN1C[C@H](F)C2]

In one embodiment, the compound is

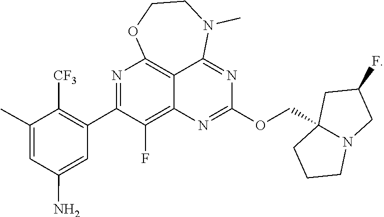 [SMILES: Cc1cc(N)cc(-c2nc3c4c(nc(OC[C@@]56CCCN5C[C@H](F)C6)nc4c2F)N(C)CCO3)c1C(F)(F)F]

Aspect 8: In one embodiment, t is 1.

Group 3.1: In one embodiment, X is N.

Group 3,1,1: In one embodiment, ring A is

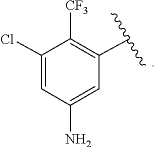 [SMILES: *c1cc(N)cc(Cl)c1C(F)(F)F]

In one embodiment, ring B is

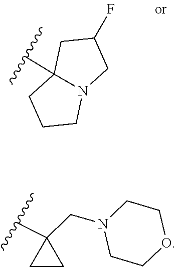 [SMILES: *C12CCCN1CC(F)C2 ; *C1(CN2CCOCC2)CC1]

Group 3,1,1,1: In one embodiment, ring B is

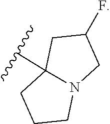 [SMILES: *C12CCCN1CC(F)C2]

In one embodiment, ring B is

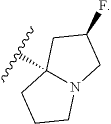 [SMILES: *[C@@]12CCCN1C[C@H](F)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl.

In one embodiment, each of R^{3a}, R^{3b}, R^{4a}, R^{4b}, R^{5a}, and R^{5b}, independently, is H, OH, CN, unsubstituted or substituted methyl, or unsubstituted or substituted ethyl.

In one embodiment, R⁶ is unsubstituted or substituted C₁₋₅alkyl, unsubstituted or substituted C₃₋₅ cycloalkyl, or unsubstituted or substituted 3-member to 5-member heterocyclyl.

In one embodiment, R⁶ is methyl, ethyl, cyclopropyl, or cyclobutyl, optionally substituted by CN or OH. In one embodiment, R⁶ is methyl. In one embodiment, R⁶ is cyclopropyl. In one embodiment, R⁶ is cyclobutyl. In one embodiment, R⁶ is 1-(methylamino)propan-2-yl.

In one embodiment, the compound is

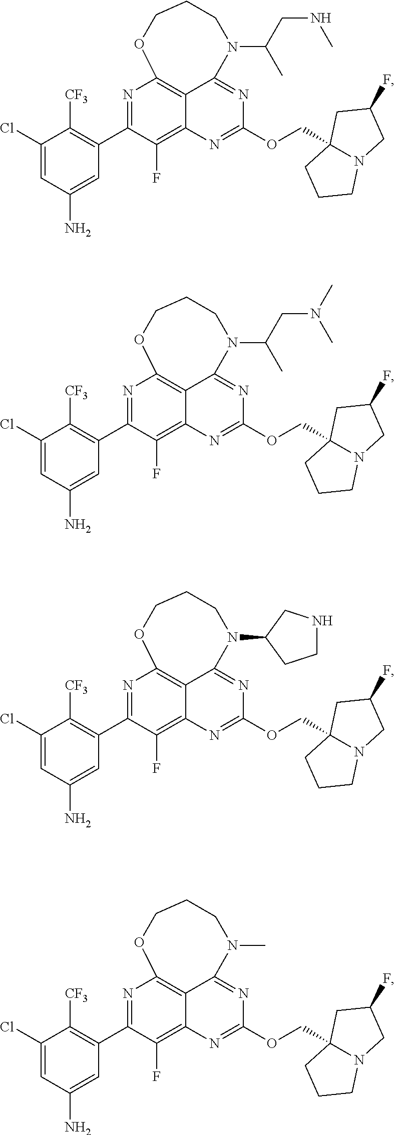

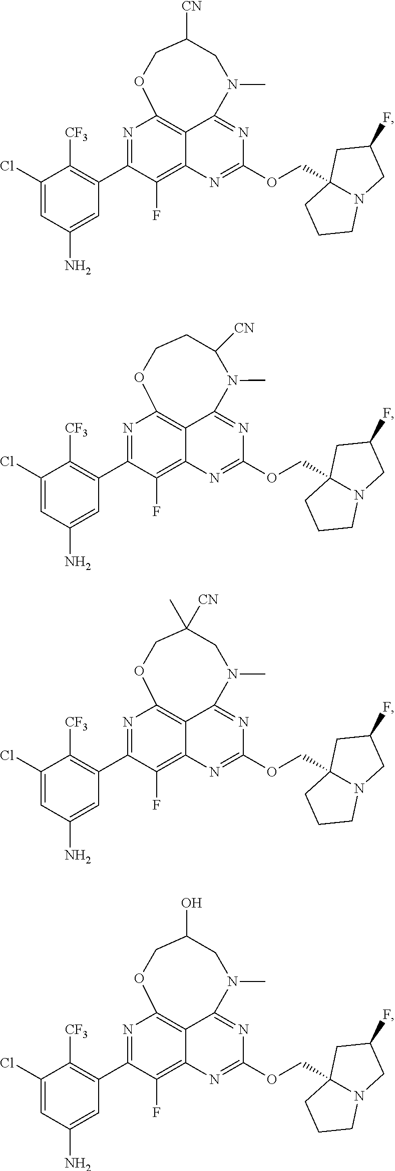

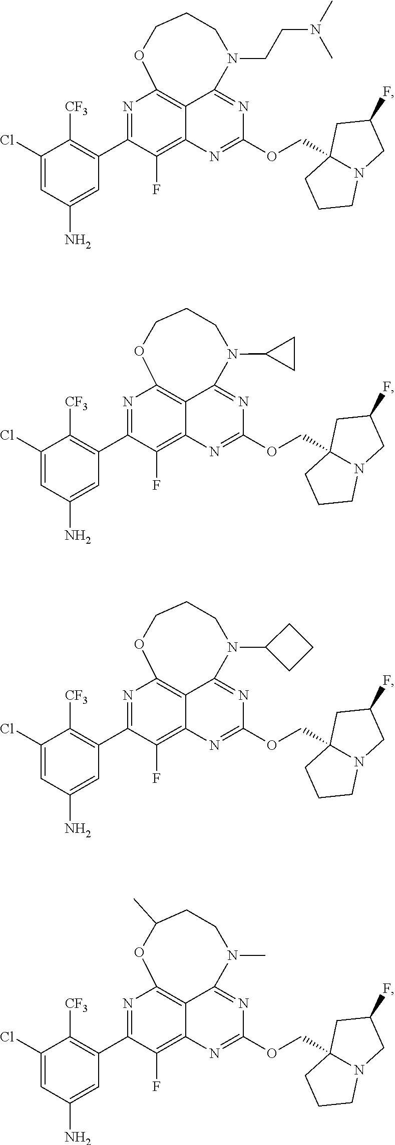

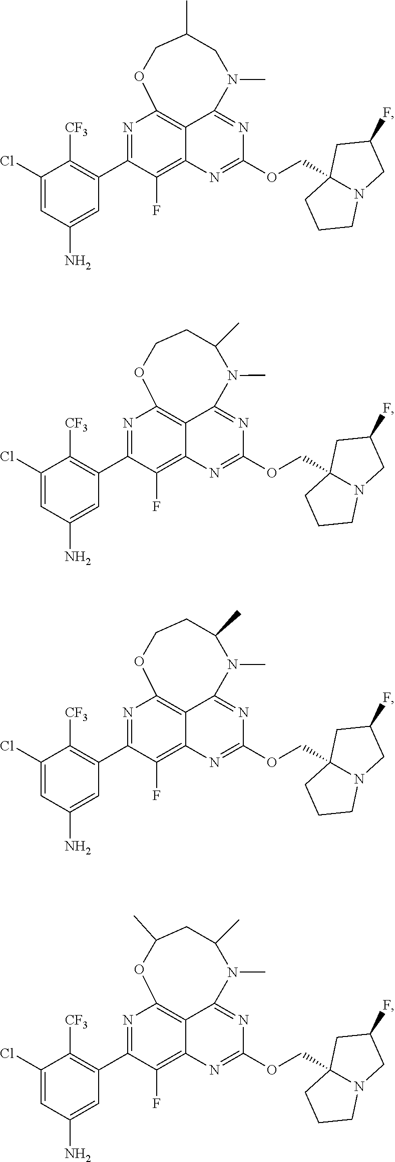

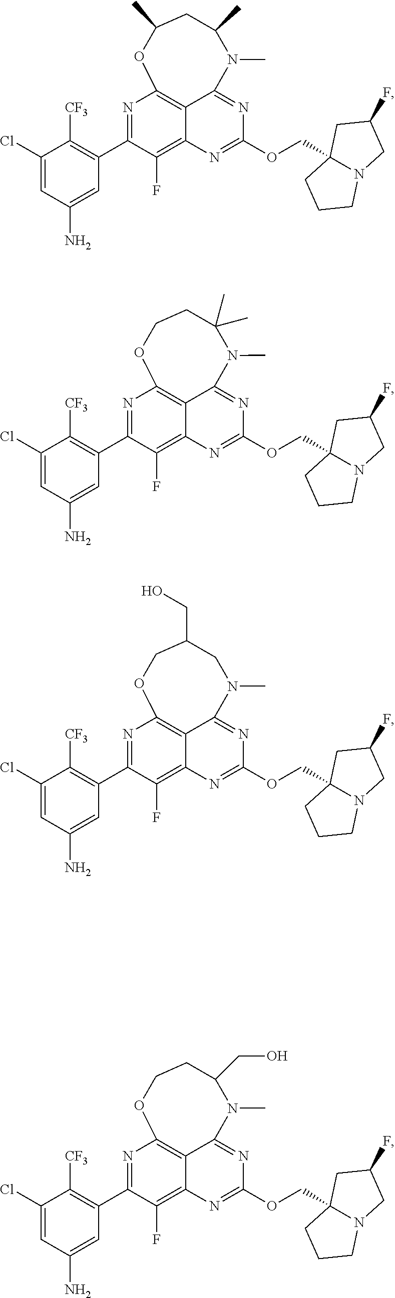

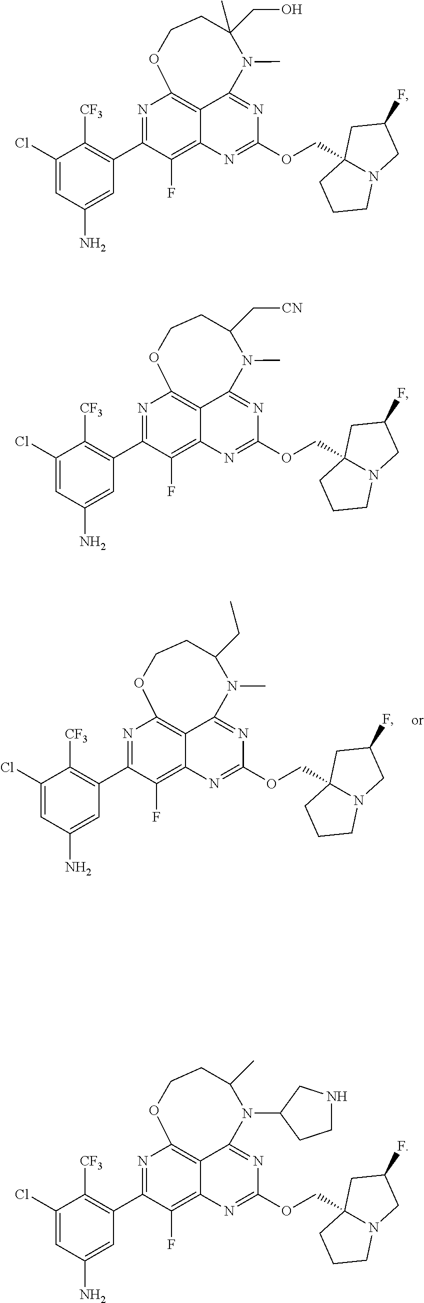

In one embodiment, R⁶ is methyl, 2-(methylamino)-ethyl, or 2-(dimethylamino)-ethyl.

In one embodiment, R⁵ and R^{5a}%, together with the atom to which they are attached, form unsubstituted or substituted C₃₋₅ cycloalkyl. In one embodiment, the C₃₋₅cycloalkyl is cyclopropyl, cyclobutyl, or cyclopentyl. In one embodiment, the heterocyclyl is cyclopropyl, or cyclobutyl. In one embodiment, the heterocyclyl is cyclopropyl. In one embodiment, the heterocyclyl is cyclobutyl.

In one embodiment, the compound is

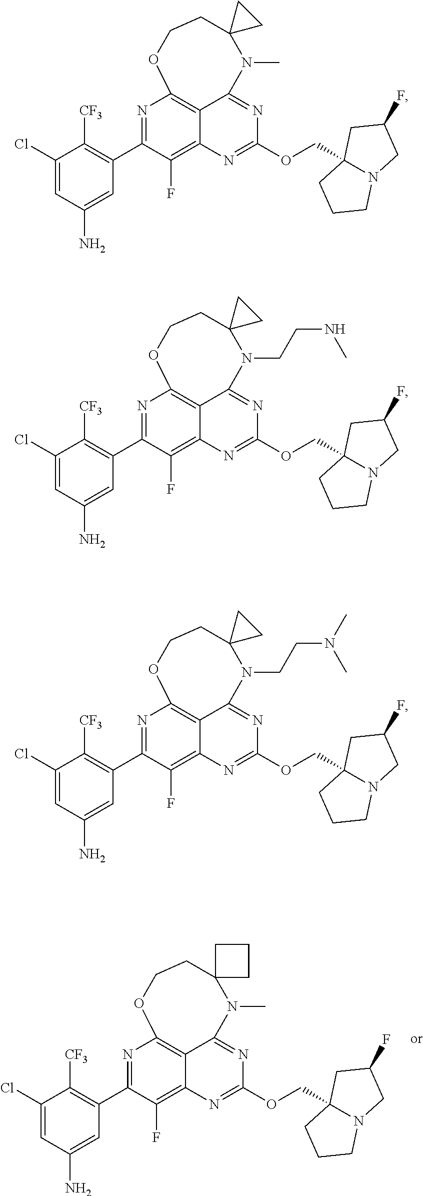

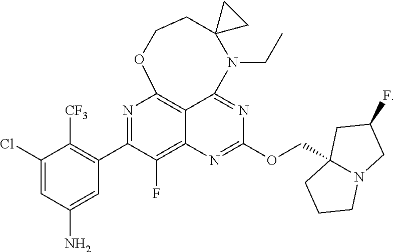

In one embodiment, R^{5a} and R^{5b}, together with the atom to which they are attached, form unsubstituted or substituted heterocyclyl. In one embodiment, the heterocyclyl is oxetanyl, tetrahydrofuranyl, tetrahydropyranyl, or dioxanyl. In one embodiment, the heterocyclyl is oxetanyl, or tetrahydrofuranyl. In one embodiment, the heterocyclyl is oxetanyl. In one embodiment, the heterocyclyl is tetrahydrofuranyl.

In one embodiment, R⁶ is methyl, 2-(methylamino)-ethyl, or 2-(dimethylamino)-ethyl.

In one embodiment, the compound is

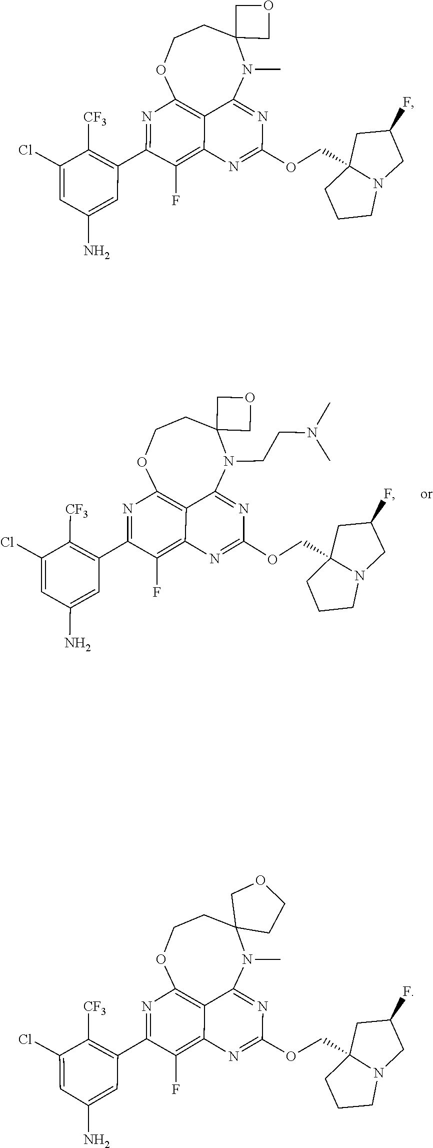 [SMILES: CN1c2nc(OC[C@@]34CCCN3C[C@H](F)C4)nc3c(F)c(-c4cc(N)cc(Cl)c4C(F)(F)F)nc(c23)OCCC12COC2 ; CN(C)CCN1c2nc(OC[C@@]34CCCN3C[C@H](F)C4)nc3c(F)c(-c4cc(N)cc(Cl)c4C(F)(F)F)nc(c23)OCCC12COC2 ; CN1c2nc(OC[C@@]34CCCN3C[C@H](F)C4)nc3c(F)c(-c4cc(N)cc(Cl)c4C(F)(F)F)nc(c23)OCCC12CCOC2]

In one embodiment, R^{4a} and R^{4b}, together with the atom to which they are attached, form unsubstituted or substituted heterocyclyl. In one embodiment, the heterocyclyl is oxetanyl, tetrahydrofuranyl, tetrahydropyranyl, or dioxanyl. In one embodiment, the heterocyclyl is oxetanyl, or tetrahydrofuranyl. In one embodiment, the heterocyclyl is tetrahydrofuranyl. In one embodiment, the heterocyclyl is oxetanyl.

In one embodiment, R⁶ is methyl.

In one embodiment, the compound is

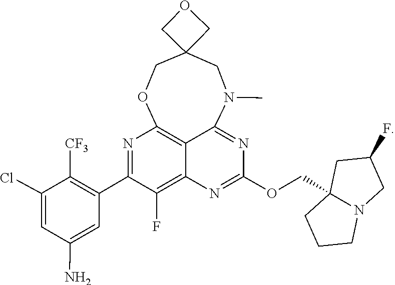 [SMILES: CN1CC2(COC2)COc2nc(-c3cc(N)cc(Cl)c3C(F)(F)F)c(F)c3nc(OC[C@@]45CCCN4C[C@H](F)C5)nc1c23]

Group 3,1,1,3: In one embodiment, ring B is

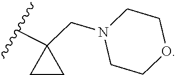 [SMILES: *C1(CN2CCOCC2)CC1]

In one embodiment, R⁶ is unsubstituted or substituted C₁₋₈ alkyl, unsubstituted or substituted C₃₋₅cycloalkyl, or unsubstituted or substituted 3-member to 5-member heterocyclyl.

In one embodiment, R⁶ is methyl, ethyl, cyclopropyl, or cyclobutyl, optionally substituted by CN or OH. In one embodiment, R^{c} is methyl.

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl.

In one embodiment, each of R^{3a}, R^{3b}, R^{4a}, R^{4b}, R^{5a}, and R^{5b}, independently, is H, OH, CN, unsubstituted or substituted methyl, or unsubstituted or substituted ethyl. In one embodiment, each of R^{3a}, R^{3b}, R^{4a}, R^{4b}, R^{5a}, and R^{5b}, independently, is H, or methyl,

In one embodiment, the compound is

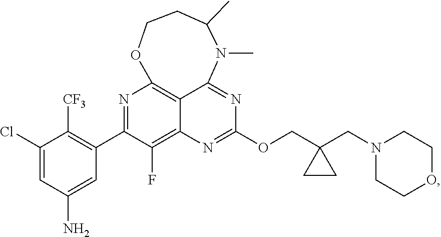 [SMILES: CC1CCOc2nc(-c3cc(N)cc(Cl)c3C(F)(F)F)c(F)c3nc(OCC4(CN5CCOCC5)CC4)nc(c23)N1C]

In one embodiment, ring A is,

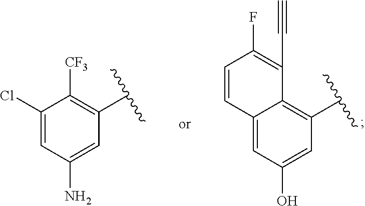 [SMILES: *c1cc(N)cc(Cl)c1C(F)(F)F ; *c1cc(O)cc2ccc(F)c(C#C)c12]

and ring B is,

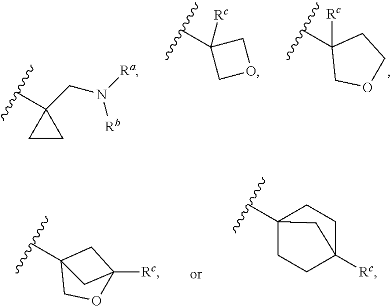

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl.

Group 3,1,1,6: In one embodiment, ring B is

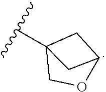 [SMILES: *C12COC(C1)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl.

In one embodiment, each of R^{3a}, R^{3b}, R^{4a}, R^{4b}, R^{5a}, and R^{5b}, independently, is H, OH, CN, unsubstituted or substituted methyl, or unsubstituted or substituted ethyl. In one embodiment, each of R^{3a}, R^{3b}, R^{4a}, R^{4b}, R^{5a}, and R^{5b}, independently, is H, or CN,

In one embodiment, R⁶ is unsubstituted or substituted C₁₋₅alkyl, unsubstituted or substituted C₃₋₅ cycloalkyl, or unsubstituted or substituted 3-member to 5-member heterocyclyl.

In one embodiment, R⁶ is methyl, ethyl, cyclopropyl, or cyclobutyl, optionally substituted by CN or OH. In one embodiment, R⁶ is 2-(dimethylamino)ethyl, or 1-(methylamino)propan-2-yl.

In one embodiment, the compound is

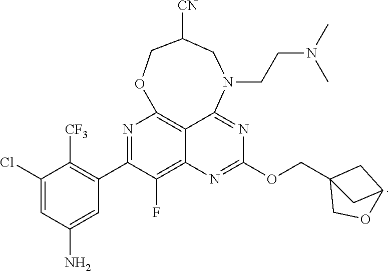 [SMILES: CN(C)CCN1CC(C#N)COc2nc(-c3cc(N)cc(Cl)c3C(F)(F)F)c(F)c3nc(OCC45COC(C4)C5)nc1c23]

Group 3.1.2: In one embodiment, ring A is

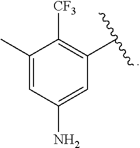 [SMILES: *c1cc(N)cc(C)c1C(F)(F)F]

In one embodiment, ring B is

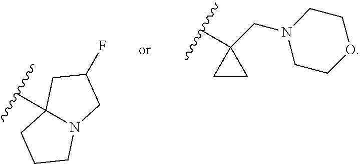 [SMILES: *C12CCCN1CC(F)C2 ; *C1(CN2CCOCC2)CC1]

Group 3.1.2.1: In one embodiment, ring B is

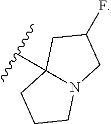 [SMILES: *C12CCCN1CC(F)C2]

In one embodiment, ring B is

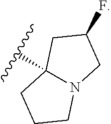 [SMILES: *[C@@]12CCCN1C[C@H](F)C2]

In one embodiment, R⁶ is unsubstituted or substituted C₁₋₅alkyl, unsubstituted or substituted C₃₋₅cycloalkyl, or unsubstituted or substituted 3-member to 5-member heterocyclyl.

In one embodiment, R⁶ is methyl, or pyrrolidinyl. In one embodiment, R⁶ is methyl. In one embodiment, R⁶ is pyrrolidinyl.

In one embodiment, the compound is

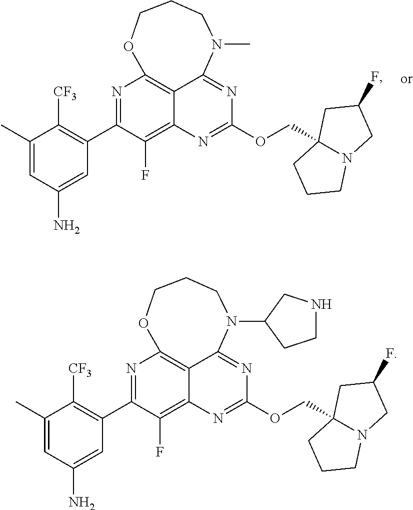 [SMILES: Cc1cc(N)cc(-c2nc3c4c(nc(OC[C@@]56CCCN5C[C@H](F)C6)nc4c2F)N(C)CCCO3)c1C(F)(F)F ; Cc1cc(N)cc(-c2nc3c4c(nc(OC[C@@]56CCCN5C[C@H](F)C6)nc4c2F)N(C2CCNC2)CCCO3)c1C(F)(F)F]

Group 3.1.4: In one embodiment, ring A is

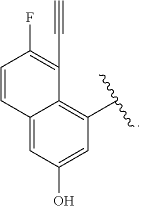 [SMILES: *c1cc(O)cc2ccc(F)c(C#C)c12]

In one embodiment, ring B is

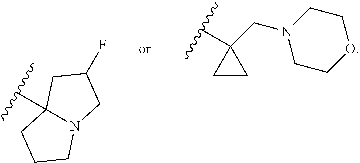 [SMILES: *C12CCCN1CC(F)C2 ; *C1(CN2CCOCC2)CC1]

Group 3.1.4.1: In one embodiment, ring B is

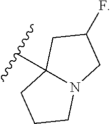 [SMILES: *C12CCCN1CC(F)C2]

In one embodiment, ring B is

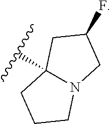 [SMILES: *[C@@]12CCCN1C[C@H](F)C2]

In one embodiment, R⁶ is unsubstituted or substituted C₁₋₈ alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl. In one embodiment, R⁶ is cyclopropyl. In one embodiment, R⁶ is cyclobutyl.

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5b}, independently, is H, or methyl.

In one embodiment, the compound is

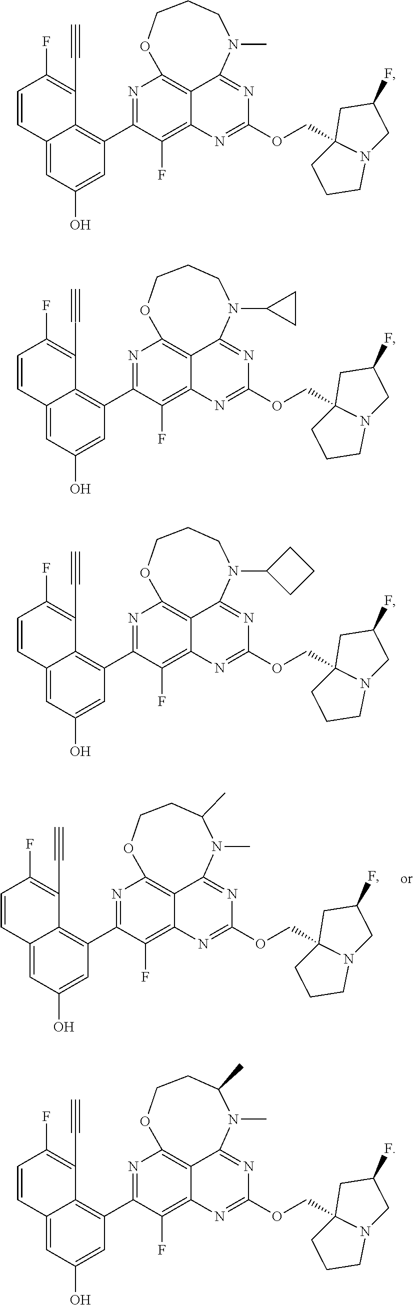 [SMILES: C#Cc1c(F)ccc2cc(O)cc(-c3nc4c5c(nc(OC[C@@]67CCCN6C[C@H](F)C7)nc5c3F)N(C)CCCO4)c12 ; C#Cc1c(F)ccc2cc(O)cc(-c3nc4c5c(nc(OC[C@@]67CCCN6C[C@H](F)C7)nc5c3F)N(C3CC3)CCCO4)c12 ; C#Cc1c(F)ccc2cc(O)cc(-c3nc4c5c(nc(OC[C@@]67CCCN6C[C@H](F)C7)nc5c3F)N(C3CCC3)CCCO4)c12 ; C#Cc1c(F)ccc2cc(O)cc(-c3nc4c5c(nc(OC[C@@]67CCCN6C[C@H](F)C7)nc5c3F)N(C)C(C)CCO4)c12 ; C#Cc1c(F)ccc2cc(O)cc(-c3nc4c5c(nc(OC[C@@]67CCCN6C[C@H](F)C7)nc5c3F)N(C)[C@H](C)CCO4)c12]

Group 3.1.5: In one embodiment, ring A is

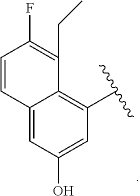 [SMILES: *c1cc(O)cc2ccc(F)c(CC)c12]

In one embodiment, ring B is

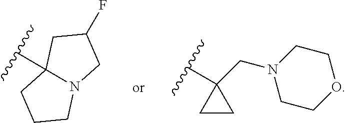 [SMILES: *C12CCCN1CC(F)C2 ; *C1(CN2CCOCC2)CC1]

Group 3.1.5.1: In one embodiment, ring B is

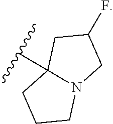 [SMILES: *C12CCCN1CC(F)C2]

In one embodiment, ring B is

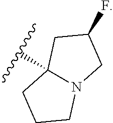 [SMILES: *[C@@]12CCCN1C[C@H](F)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5b}, independently, is H, or methyl.

In one embodiment, R⁶ is unsubstituted or substituted C₁₋₈ alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl.

In one embodiment, the compound is

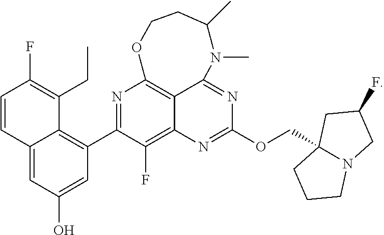 [SMILES: CCc1c(F)ccc2cc(O)cc(-c3nc4c5c(nc(OC[C@@]67CCCN6C[C@H](F)C7)nc5c3F)N(C)C(C)CCO4)c12]

Group 3.2: In one embodiment, X is C—H, C—F, C-Cl, or C—CF₃.

Group 3.2.4: In one embodiment, X is C—H.

In one embodiment, ring A is

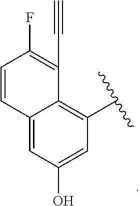 [SMILES: *c1cc(O)cc2ccc(F)c(C#C)c12]

In one embodiment, ring B is

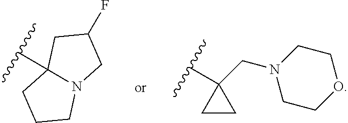 [SMILES: *C12CCCN1CC(F)C2 ; *C1(CN2CCOCC2)CC1]

Group 3.2.4.1: In one embodiment, R⁶ is methyl.

In one embodiment, ring B is

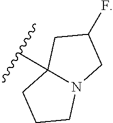 [SMILES: *C12CCCN1CC(F)C2]

In one embodiment, the compound is

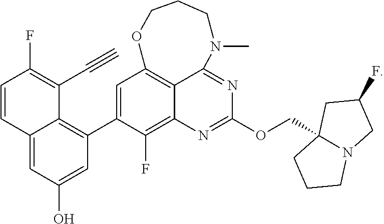 [SMILES: C#Cc1c(F)ccc2cc(O)cc(-c3cc4c5c(nc(OC[C@@]67CCCN6C[C@H](F)C7)nc5c3F)N(C)CCCO4)c12]

Group 3.2.6.1: In one embodiment, X is C—H.

In one embodiment, ring A is

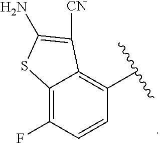 [SMILES: *c1ccc(F)c2sc(N)c(C#N)c12]

In one embodiment, ring B is

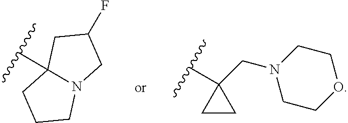 [SMILES: *C12CCCN1CC(F)C2 ; *C1(CN2CCOCC2)CC1]

In one embodiment, ring B is

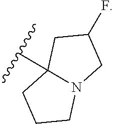 [SMILES: *C12CCCN1CC(F)C2]

In one embodiment, R⁶ is methyl.

In one embodiment, the compound is

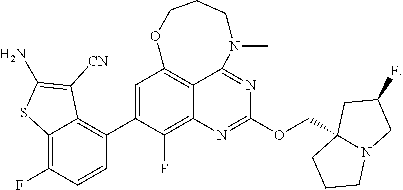 [SMILES: CN1CCCOc2cc(-c3ccc(F)c4sc(N)c(C#N)c34)c(F)c3nc(OC[C@@]45CCCN4C[C@H](F)C5)nc1c23]

Group 3.3.4: In one embodiment, X is C—F.

In one embodiment, ring A is

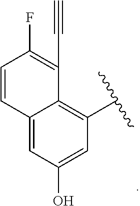 [SMILES: *c1cc(O)cc2ccc(F)c(C#C)c12]

In one embodiment, ring B is

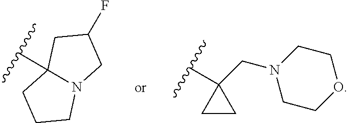 [SMILES: *C12CCCN1CC(F)C2 ; *C1(CN2CCOCC2)CC1]

In one embodiment, ring B is

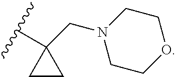 [SMILES: *C1(CN2CCOCC2)CC1]

In one embodiment, R^{5a} is methyl. In one embodiment, R⁶ is methyl.

In one embodiment, the compound is

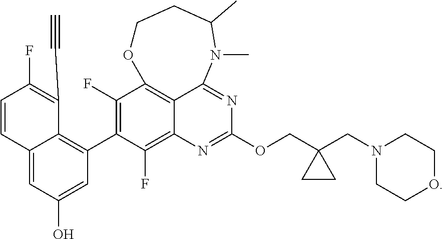 [SMILES: C#Cc1c(F)ccc2cc(O)cc(-c3c(F)c4c5c(nc(OCC6(CN7CCOCC7)CC6)nc5c3F)N(C)C(C)CCO4)c12]

Group 3.4.2.1: In one embodiment, X is C—Cl. In one embodiment, ring A is

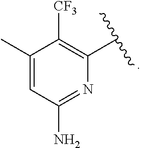 [SMILES: *c1nc(N)cc(C)c1C(F)(F)F]

In one embodiment, ring A is

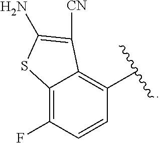 [SMILES: *c1ccc(F)c2sc(N)c(C#N)c12]

In one embodiment, ring B is

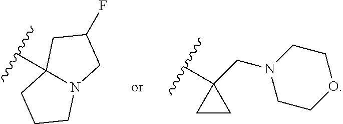 [SMILES: *C12CCCN1CC(F)C2 ; *C1(CN2CCOCC2)CC1]

In one embodiment, ring B is

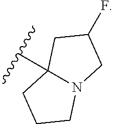 [SMILES: *C12CCCN1CC(F)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5b}, independently, is H, or methyl. In one embodiment, R^{5a} is methyl. In one embodiment, R^{5a} is methylaminomethyl.

In one embodiment, R⁶ is unsubstituted or substituted C₁₋₈ alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl.

In one embodiment, the compound is

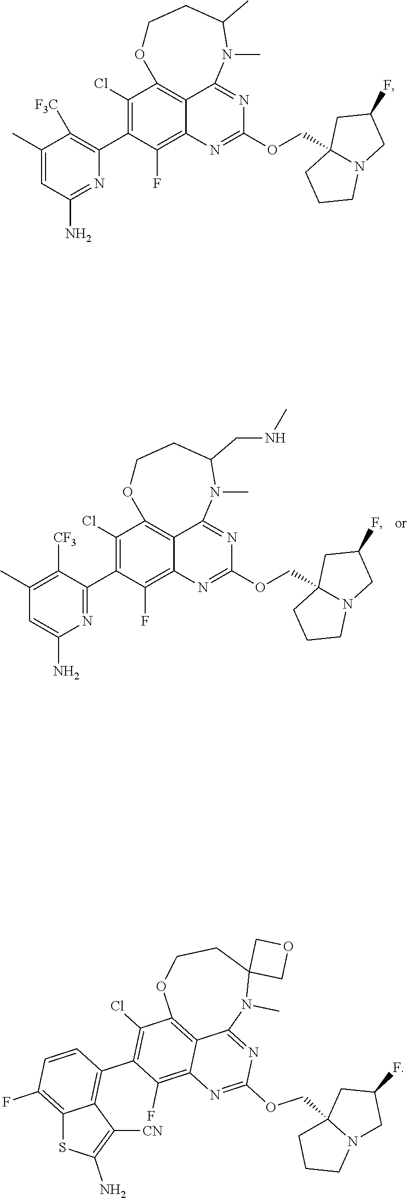 [SMILES: Cc1cc(N)nc(-c2c(Cl)c3c4c(nc(OC[C@@]56CCCN5C[C@H](F)C6)nc4c2F)N(C)C(C)CCO3)c1C(F)(F)F ; CNCC1CCOc2c(Cl)c(-c3nc(N)cc(C)c3C(F)(F)F)c(F)c3nc(OC[C@@]45CCCN4C[C@H](F)C5)nc(c23)N1C ; CN1c2nc(OC[C@@]34CCCN3C[C@H](F)C4)nc3c(F)c(-c4ccc(F)c5sc(N)c(C#N)c45)c(Cl)c(c23)OCCC12COC2]

Group 3.4.4.1: In one embodiment, X is C—Cl.

In one embodiment, ring A is

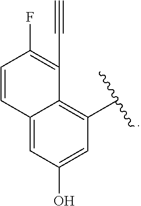 [SMILES: *c1cc(O)cc2ccc(F)c(C#C)c12]

In one embodiment, R^{5a} is methyl. In one embodiment, R⁶ is methyl.

In one embodiment, the compound is

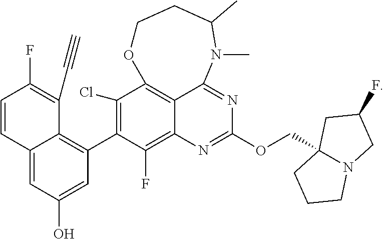 [SMILES: C#Cc1c(F)ccc2cc(O)cc(-c3c(Cl)c4c5c(nc(OC[C@@]67CCCN6C[C@H](F)C7)nc5c3F)N(C)C(C)CCO4)c12]

Group 3.4.4.3: In one embodiment, X is C—Cl. In one embodiment, ring A is

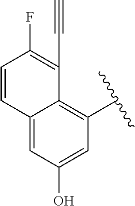 [SMILES: *c1cc(O)cc2ccc(F)c(C#C)c12]

In one embodiment, ring B is

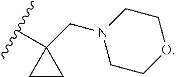 [SMILES: *C1(CN2CCOCC2)CC1]

In one embodiment, R^{5a} is methyl. In one embodiment, R⁶ is methyl.

In one embodiment, the compound is

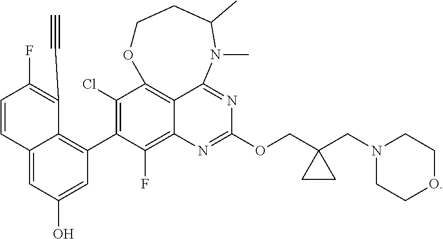 [SMILES: C#Cc1c(F)ccc2cc(O)cc(-c3c(Cl)c4c5c(nc(OCC6(CN7CCOCC7)CC6)nc5c3F)N(C)C(C)CCO4)c12]

Group 3.4.5.3: In one embodiment, X is C—Cl.

In one embodiment, ring A is

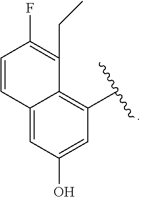 [SMILES: *c1cc(O)cc2ccc(F)c(CC)c12]

In one embodiment, ring B is

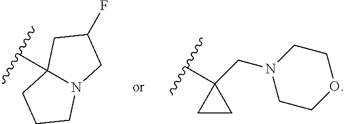 [SMILES: *C12CCCN1CC(F)C2 ; *C1(CN2CCOCC2)CC1]

In one embodiment, ring B is

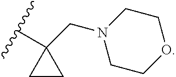 [SMILES: *C1(CN2CCOCC2)CC1]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5b}, independently, is H, or unsubstituted or substituted methyl. In one embodiment, R^{5a} is methylaminomethyl.

In one embodiment, R⁶ is unsubstituted or substituted C₁₋₅alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl.

In one embodiment, the compound is

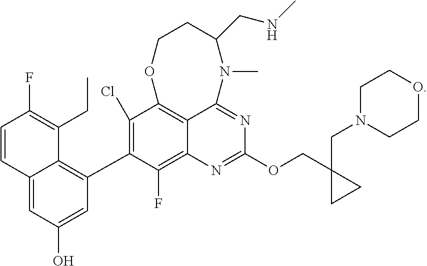 [SMILES: CCc1c(F)ccc2cc(O)cc(-c3c(Cl)c4c5c(nc(OCC6(CN7CCOCC7)CC6)nc5c3F)N(C)C(CNC)CCO4)c12]

Group 3.4.6.1: In one embodiment, X is C—Cl.

In one embodiment, ring A is

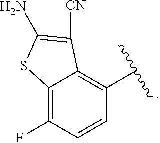 [SMILES: *c1ccc(F)c2sc(N)c(C#N)c12]

In one embodiment, ring B is

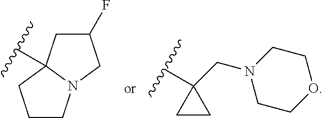 [SMILES: *C12CCCN1CC(F)C2 ; *C1(CN2CCOCC2)CC1]

In one embodiment, ring B is

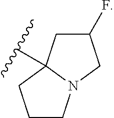 [SMILES: *C12CCCN1CC(F)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5a}%, independently, is H, or methyl. In one embodiment, R^{5a} is methyl.

In one embodiment, R⁶ is unsubstituted or substituted C₁₋₈ alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl.

In one embodiment, the compound is

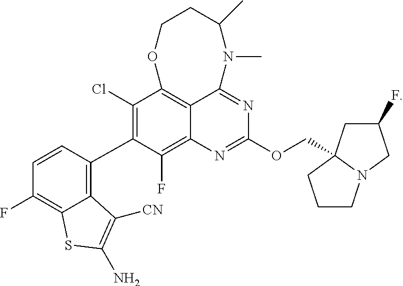 [SMILES: CC1CCOc2c(Cl)c(-c3ccc(F)c4sc(N)c(C#N)c34)c(F)c3nc(OC[C@@]45CCCN4C[C@H](F)C5)nc(c23)N1C]

Group 3.4.6.3: In one embodiment, X is C—Cl. In one embodiment, ring A is

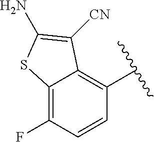 [SMILES: *c1ccc(F)c2sc(N)c(C#N)c12]

In one embodiment, ring B is

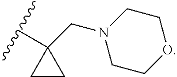 [SMILES: *C1(CN2CCOCC2)CC1]

In one embodiment, R^{5a} is methyl. In one embodiment, R⁶ is methyl.

In one embodiment, the compound is

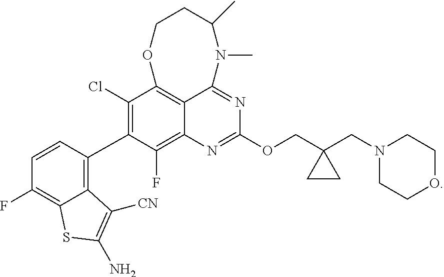 [SMILES: CC1CCOc2c(Cl)c(-c3ccc(F)c4sc(N)c(C#N)c34)c(F)c3nc(OCC4(CN5CCOCC5)CC4)nc(c23)N1C]

Group 3.4.6.6: In one embodiment, X is C—Cl.

In one embodiment, ring A is

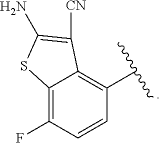 [SMILES: *c1ccc(F)c2sc(N)c(C#N)c12]

In one embodiment, ring B is

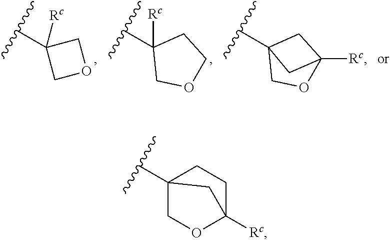

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl.

In one embodiment, ring B is

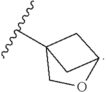 [SMILES: *C12COC(C1)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5b}, independently, is H, or unsubstituted or substituted methyl. In one embodiment, R^{5a} is methylaminomethyl.

In one embodiment, R⁶ is unsubstituted or substituted C₁₋₅alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl.

In one embodiment, the compound is

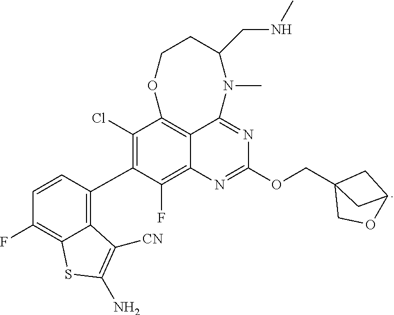 [SMILES: CNCC1CCOc2c(Cl)c(-c3ccc(F)c4sc(N)c(C#N)c34)c(F)c3nc(OCC45COC(C4)C5)nc(c23)N1C]

Group 3.4.7.1: In one embodiment, X is C—Cl.

In one embodiment, ring A is

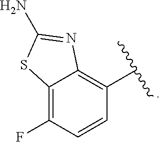 [SMILES: *c1ccc(F)c2sc(N)nc12]

In one embodiment, ring B is

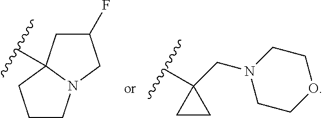 [SMILES: *C12CCCN1CC(F)C2 ; *C1(CN2CCOCC2)CC1]

In one embodiment, ring B is

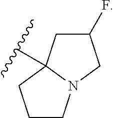 [SMILES: *C12CCCN1CC(F)C2]

In one embodiment, each of R^{3a}, R^{3b}, R^{4a}, R^{4b}, R^{5a}, and R^{5b}, independently, is H, OH, CN, unsubstituted or substituted amino, or unsubstituted or substituted C₁₋₄ alkyl. In one embodiment, each of R^{3a}, R^{3b}, R^{4a}, R^{4b}, R^{5a}, and R^{5b}, independently, is H, or unsubstituted or substituted methyl. In one embodiment, R^{5a} is methylaminomethyl.

In one embodiment, R⁶ is unsubstituted or substituted C₁₋₅alkyl, unsubstituted or substituted C₃₋₅cycloalkyl, or unsubstituted or substituted 3-member to 5-member heterocyclyl. In one embodiment, R⁶ is methyl, cyclopropyl, or cyclobutyl. In one embodiment, R⁶ is methyl.

In one embodiment, the compound is

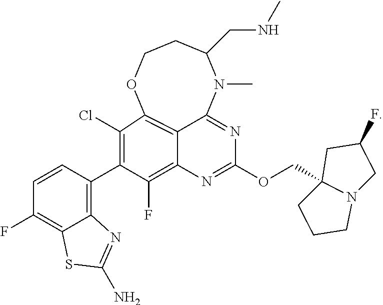 [SMILES: CNCC1CCOc2c(Cl)c(-c3ccc(F)c4sc(N)nc34)c(F)c3nc(OC[C@@]45CCCN4C[C@H](F)C5)nc(c23)N1C]

Aspect 9: In one embodiment, the compound is selected from Table 4 and Table 5.

Aspect 10: In some embodiments, the compounds provided herein has one of the following formulas:

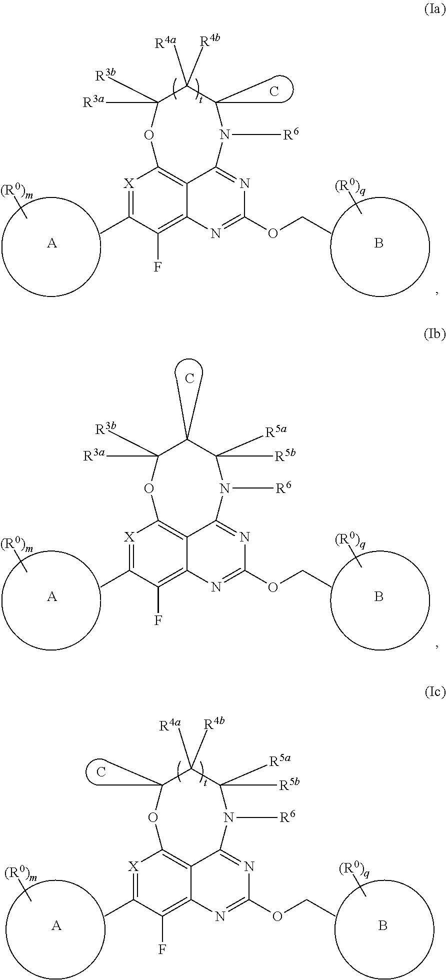

and pharmaceutically acceptable salts, tautomers, stereoisomers, enantiomers, atropisomers, isotopologues, and prodrugs thereof,
wherein:
ring C is unsubstituted or substituted C₃₋₆ cycloalkyl, or unsubstituted or substituted 3-membered to 6-membered heterocyclyl.

In one embodiment, ring C has one oxygen as the heteroatom.

In some embodiments, the compounds provided herein has one of the following formulas:

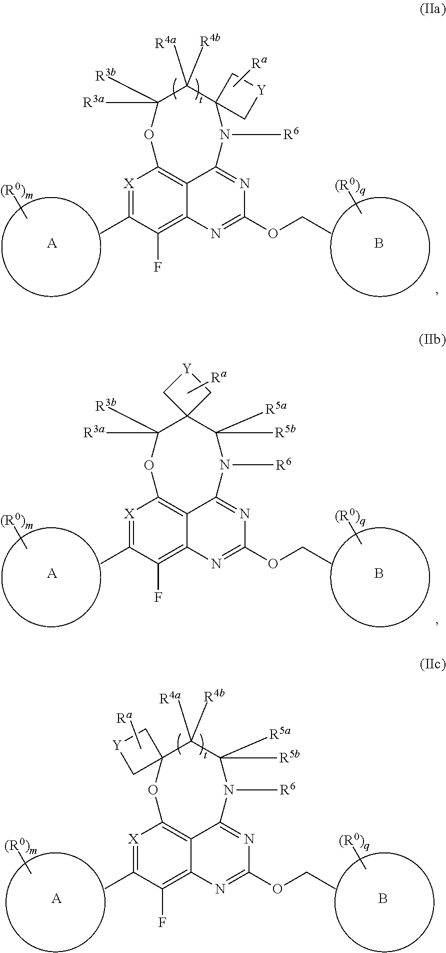

and pharmaceutically acceptable salts, tautomers, stereoisomers, enantiomers, atropisomers, isotopologues, and prodrugs thereof,
wherein:
Y is CH₂, O, NH, N—R⁹, N—C(═O)—R¹⁰, or O═S═O;
R⁹ is substituted or unsubstituted C₁₋₄ alkyl, or unsubstituted or substituted C₃₋₅ cycloalkyl;
R¹⁰ is substituted or unsubstituted C₁₋₄ alkyl, unsubstituted or substituted C₁₋₄ alkoxy, unsubstituted or substituted C₃₋₅ cycloalkyl, or unsubstituted or substituted 3-member to 5-member heterocyclyl; and
R^{a} is H, halogen, amino, —CN, —OH, unsubstituted or substituted C₁₋₄ alkyl, unsubstituted or substituted C₁₋₄ alkoxy, or unsubstituted or substituted C₁₋₄ alkylamino.

In one embodiment, Y is CH₂. In one embodiment, Y is O. In one embodiment, Y is NH.

Aspect 11: In some embodiments, the compounds provided herein have the following formula:

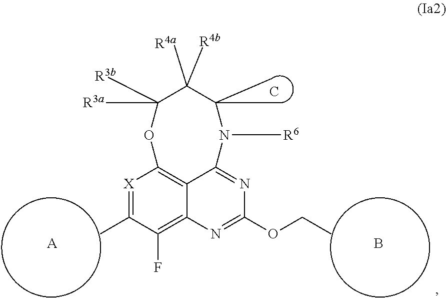

and pharmaceutically acceptable salts, tautomers, stereoisomers, enantiomers, atropisomers, isotopologues, and prodrugs thereof,
wherein:
ring C is unsubstituted or substituted C₃₋₄ cycloalkyl, or unsubstituted or substituted 3-membered to 4-membered heterocyclyl.

In one embodiment, each of R^{3a}, R^{3b}, R^{4a}, and R^{4b}, independently, is H, OH, CN, halogen, unsubstituted or substituted amino, or unsubstituted or substituted C₁₋₄ alkyl. In one embodiment, each of R^{3a}, R^{3b}, R^{4a}, and R^{4b}, independently, is H, F, or unsubstituted or substituted methyl. In one embodiment, each of R^{3a}, R^{3b}, R^{4a}, and R^{4b}, independently, is H, F, or unsubstituted methyl. In one embodiment, each of R^{3a}, R^{3b}, R^{4a}, and R^{4b}, independently, is H, or F. In one embodiment, each of R^{3a}, R^{3b}, R^{4a}, and R^{4b}, independently, is H.

In one embodiment, ring C is a cyclopropyl ring, a cyclobutyl ring, an oxetane ring, optionally substituted with OH, CN, halogen, unsubstituted or substituted amino, or unsubstituted or substituted C₁₋₄ alkyl. In one embodiment, ring C is a cyclopropyl ring, a cyclobutyl ring, an oxetane ring, optionally substituted with OH. In one embodiment, ring C is a cyclopropyl ring, a cyclobutyl ring, an oxetane ring, each of which is optionally substituted with one or more F. In one embodiment, ring C is a cyclopropyl ring, optionally substituted with one or two F. In one embodiment, ring C is an unsubstituted cyclopropyl ring. In one embodiment, ring C is an unsubstituted cyclobutyl ring.

In one embodiment, R⁶ is H, unsubstituted or substituted C₁₋₄ alkyl, unsubstituted or substituted C₃₋₄ cycloalkyl, or unsubstituted or substituted 3-membered to 4-membered heterocyclyl. In one embodiment, R⁶ is methyl, ethyl, 2-dimethylamino-ethyl, or cyclopropyl. In one embodiment, R⁶ is methyl.

In one embodiment, X is N. In one embodiment, X is C—Cl.

In one embodiment, ring A is

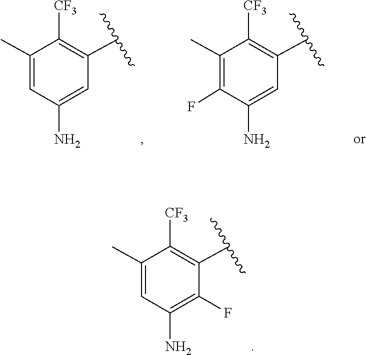 [SMILES: *c1cc(N)cc(C)c1C(F)(F)F ; *c1cc(N)c(F)c(C)c1C(F)(F)F ; *c1c(F)c(N)cc(C)c1C(F)(F)F]

In one embodiment, ring A is

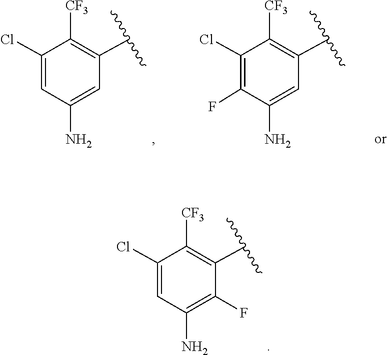 [SMILES: *c1cc(N)cc(Cl)c1C(F)(F)F ; *c1cc(N)c(F)c(Cl)c1C(F)(F)F ; *c1c(F)c(N)cc(Cl)c1C(F)(F)F]

In one embodiment, ring A is

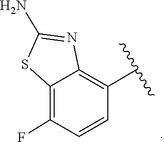 [SMILES: *c1ccc(F)c2sc(N)nc12]

In one embodiment, ring A is

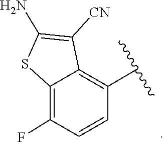 [SMILES: *c1ccc(F)c2sc(N)c(C#N)c12]

In one embodiment, ring A is

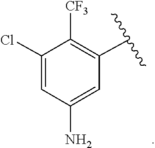 [SMILES: *c1cc(N)cc(Cl)c1C(F)(F)F]

In one embodiment, ring A is,

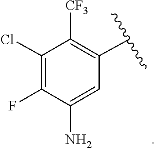 [SMILES: *c1cc(N)c(F)c(Cl)c1C(F)(F)F]

In one embodiment, ring B is

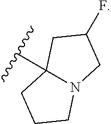 [SMILES: *C12CCCN1CC(F)C2]

In one embodiment, ring B is

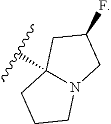 [SMILES: *[C@@]12CCCN1C[C@H](F)C2]

In one embodiment, ring B is

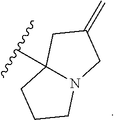 [SMILES: *C12CCCN1CC(=C)C2]

In one embodiment, ring B is

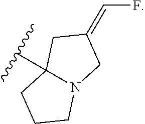 [SMILES: *C12CCCN1C/C(=C\F)C2]

In one embodiment, ring B is

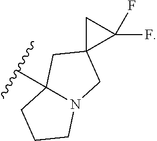 [SMILES: *C12CCCN1CC1(C2)CC1(F)F]

In one embodiment, ring B is

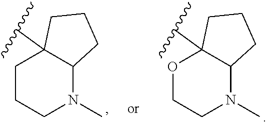 [SMILES: *C12CCCC1N(C)CCC2 ; *C12CCCC1N(C)CCO2]

In one embodiment, ring B is

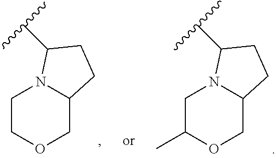 [SMILES: *C1CCC2COCCN12 ; *C1CCC2COC(C)CN12]

In one embodiment, ring B is

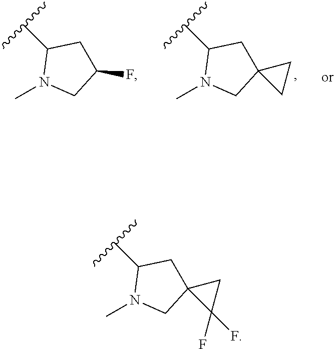

In one embodiment, ring B is

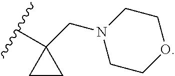 [SMILES: *C1(CN2CCOCC2)CC1]

In one embodiment, ring B is

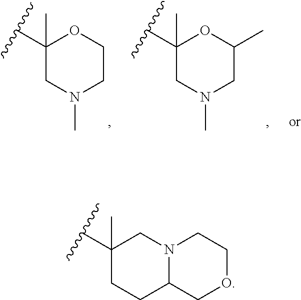

In one embodiment, ring B is

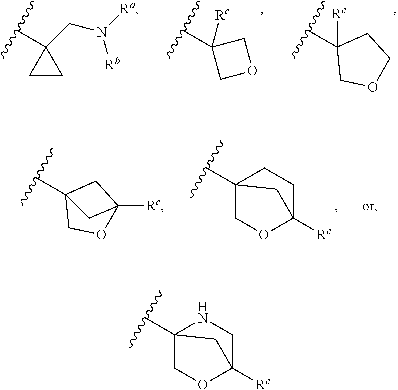

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl. In one embodiment, ring B is

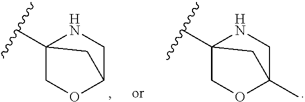

In one embodiment, the compound is

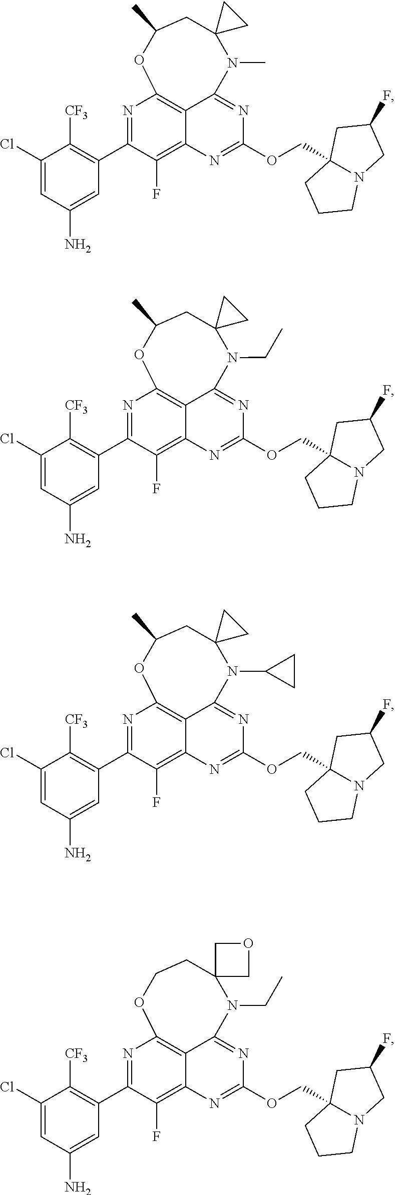

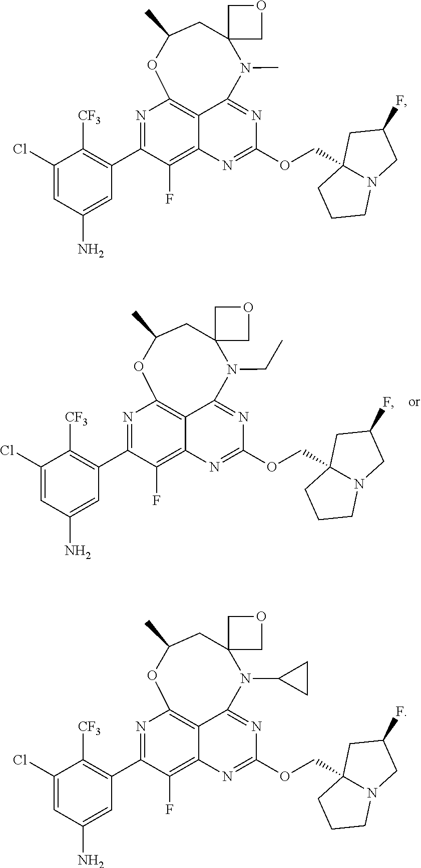

In one embodiment, the compound is

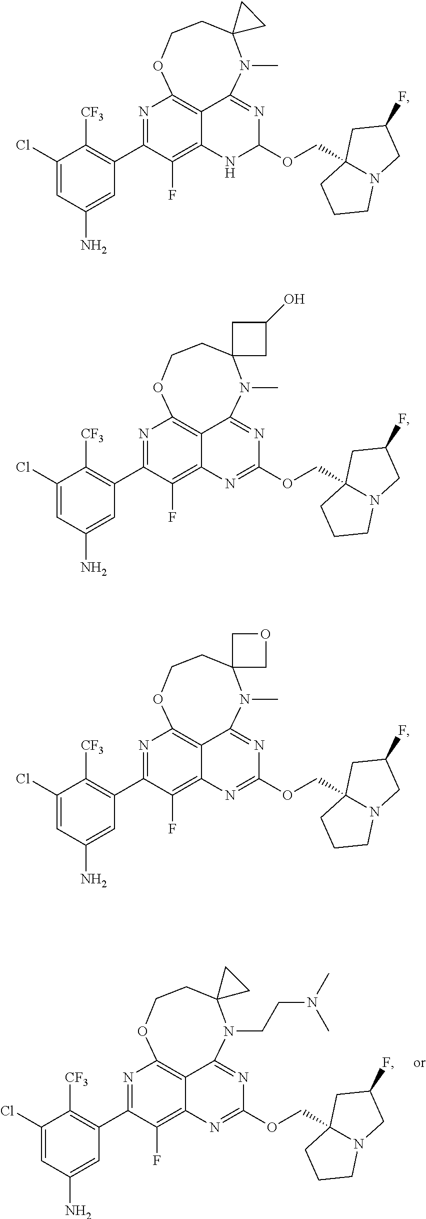 [SMILES: CN1C2=NC(OC[C@@]34CCCN3C[C@H](F)C4)Nc3c(F)c(-c4cc(N)cc(Cl)c4C(F)(F)F)nc(c32)OCCC12CC2 ; CN1c2nc(OC[C@@]34CCCN3C[C@H](F)C4)nc3c(F)c(-c4cc(N)cc(Cl)c4C(F)(F)F)nc(c23)OCCC12CC(O)C2 ; CN1c2nc(OC[C@@]34CCCN3C[C@H](F)C4)nc3c(F)c(-c4cc(N)cc(Cl)c4C(F)(F)F)nc(c23)OCCC12COC2 ; CN(C)CCN1c2nc(OC[C@@]34CCCN3C[C@H](F)C4)nc3c(F)c(-c4cc(N)cc(Cl)c4C(F)(F)F)nc(c23)OCCC12CC2]

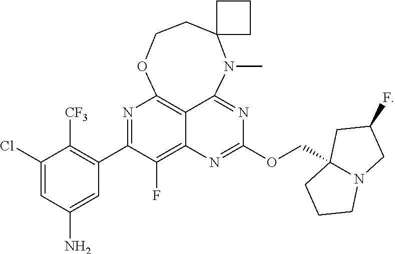 [SMILES: CN1c2nc(OC[C@@]34CCCN3C[C@H](F)C4)nc3c(F)c(-c4cc(N)cc(Cl)c4C(F)(F)F)nc(c23)OCCC12CCC2]

In one embodiment, ring A is

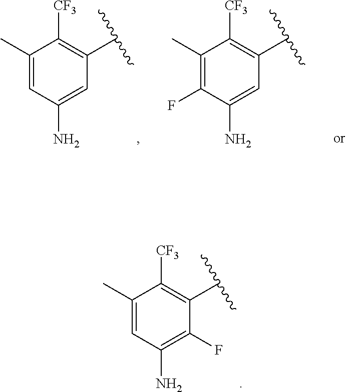 [SMILES: *c1cc(N)cc(C)c1C(F)(F)F ; *c1cc(N)c(F)c(C)c1C(F)(F)F ; *c1c(F)c(N)cc(C)c1C(F)(F)F]

In one embodiment, ring A is

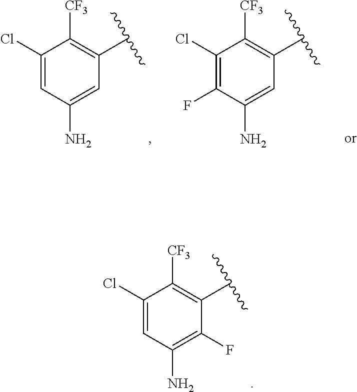 [SMILES: *c1cc(N)cc(Cl)c1C(F)(F)F ; *c1cc(N)c(F)c(Cl)c1C(F)(F)F ; *c1c(F)c(N)cc(Cl)c1C(F)(F)F]

In one embodiment, the compound is

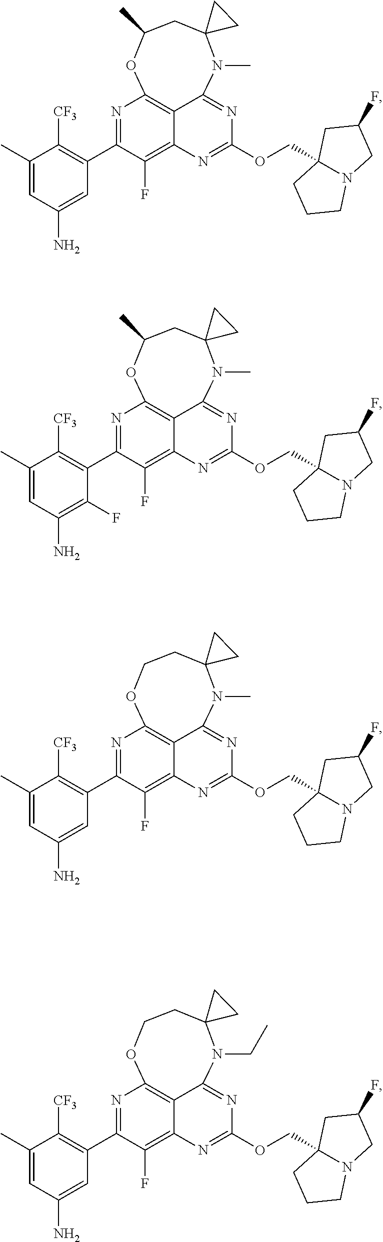 [SMILES: Cc1cc(N)cc(-c2nc3c4c(nc(OC[C@@]56CCCN5C[C@H](F)C6)nc4c2F)N(C)C2(CC2)C[C@H](C)O3)c1C(F)(F)F ; Cc1cc(N)c(F)c(-c2nc3c4c(nc(OC[C@@]56CCCN5C[C@H](F)C6)nc4c2F)N(C)C2(CC2)C[C@H](C)O3)c1C(F)(F)F ; Cc1cc(N)cc(-c2nc3c4c(nc(OC[C@@]56CCCN5C[C@H](F)C6)nc4c2F)N(C)C2(CCO3)CC2)c1C(F)(F)F ; CCN1c2nc(OC[C@@]34CCCN3C[C@H](F)C4)nc3c(F)c(-c4cc(N)cc(C)c4C(F)(F)F)nc(c23)OCCC12CC2]

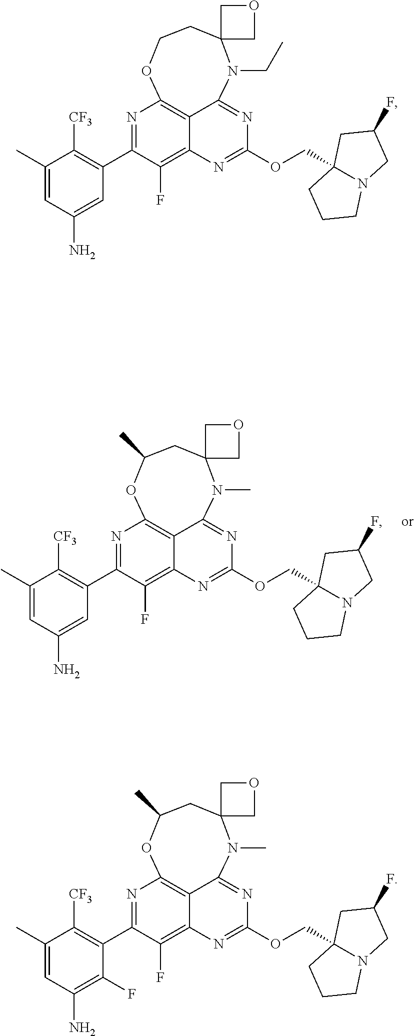 [SMILES: CCN1c2nc(OC[C@@]34CCCN3C[C@H](F)C4)nc3c(F)c(-c4cc(N)cc(C)c4C(F)(F)F)nc(c23)OCCC12COC2 ; Cc1cc(N)cc(-c2nc3c4c(nc(OC[C@@]56CCCN5C[C@H](F)C6)nc4c2F)N(C)C2(COC2)C[C@H](C)O3)c1C(F)(F)F ; Cc1cc(N)c(F)c(-c2nc3c4c(nc(OC[C@@]56CCCN5C[C@H](F)C6)nc4c2F)N(C)C2(COC2)C[C@H](C)O3)c1C(F)(F)F]

In one embodiment, the compound is

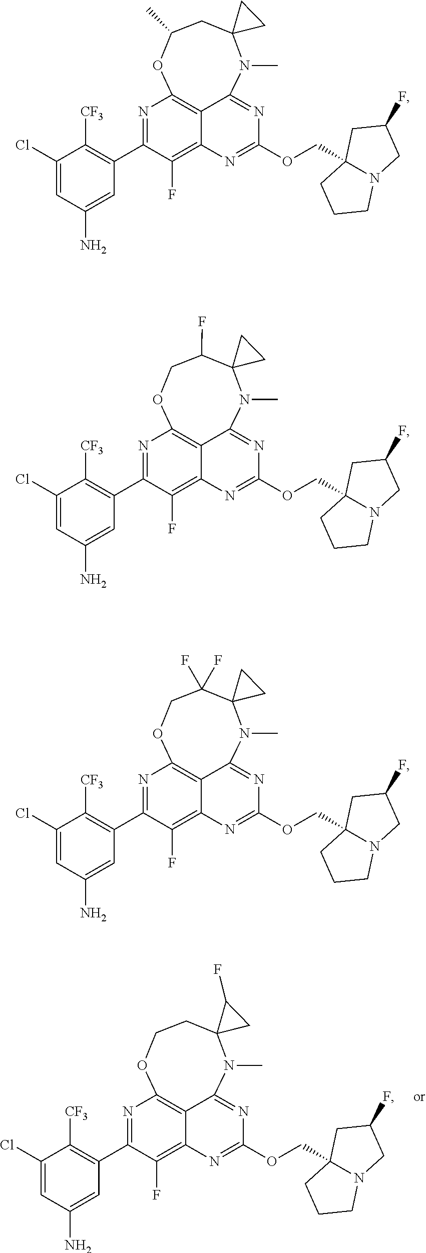 [SMILES: C[C@@H]1CC2(CC2)N(C)c2nc(OC[C@@]34CCCN3C[C@H](F)C4)nc3c(F)c(-c4cc(N)cc(Cl)c4C(F)(F)F)nc(c23)O1 ; CN1c2nc(OC[C@@]34CCCN3C[C@H](F)C4)nc3c(F)c(-c4cc(N)cc(Cl)c4C(F)(F)F)nc(c23)OCC(F)C12CC2 ; CN1c2nc(OC[C@@]34CCCN3C[C@H](F)C4)nc3c(F)c(-c4cc(N)cc(Cl)c4C(F)(F)F)nc(c23)OCC(F)(F)C12CC2 ; CN1c2nc(OC[C@@]34CCCN3C[C@H](F)C4)nc3c(F)c(-c4cc(N)cc(Cl)c4C(F)(F)F)nc(c23)OCCC12CC2F]

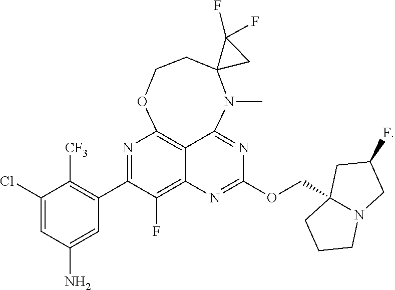 [SMILES: CN1c2nc(OC[C@@]34CCCN3C[C@H](F)C4)nc3c(F)c(-c4cc(N)cc(Cl)c4C(F)(F)F)nc(c23)OCCC12CC2(F)F]

In one embodiment, the compound is

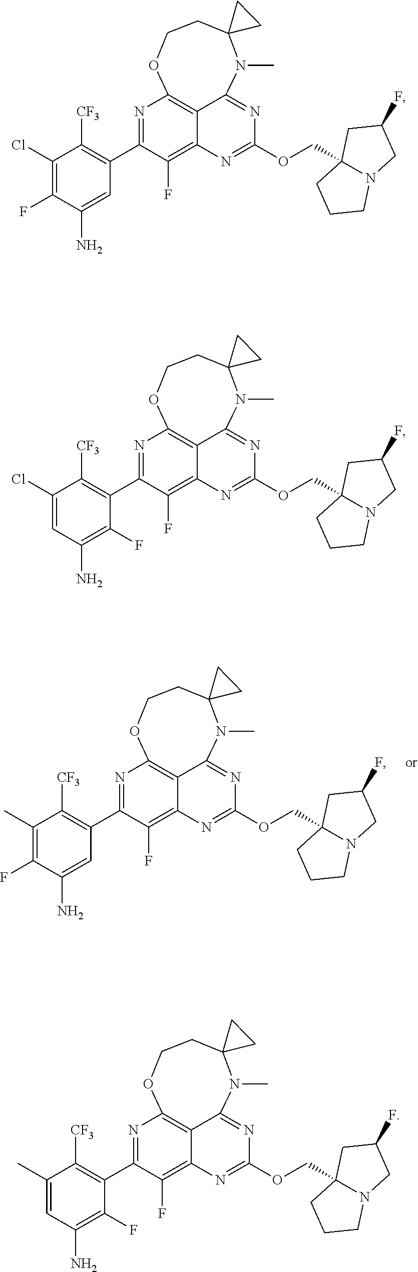 [SMILES: CN1c2nc(OC[C@@]34CCCN3C[C@H](F)C4)nc3c(F)c(-c4cc(N)c(F)c(Cl)c4C(F)(F)F)nc(c23)OCCC12CC2 ; CN1c2nc(OC[C@@]34CCCN3C[C@H](F)C4)nc3c(F)c(-c4c(F)c(N)cc(Cl)c4C(F)(F)F)nc(c23)OCCC12CC2 ; Cc1c(F)c(N)cc(-c2nc3c4c(nc(OC[C@@]56CCCN5C[C@H](F)C6)nc4c2F)N(C)C2(CCO3)CC2)c1C(F)(F)F ; Cc1cc(N)c(F)c(-c2nc3c4c(nc(OC[C@@]56CCCN5C[C@H](F)C6)nc4c2F)N(C)C2(CCO3)CC2)c1C(F)(F)F]

In one embodiment, the compound is

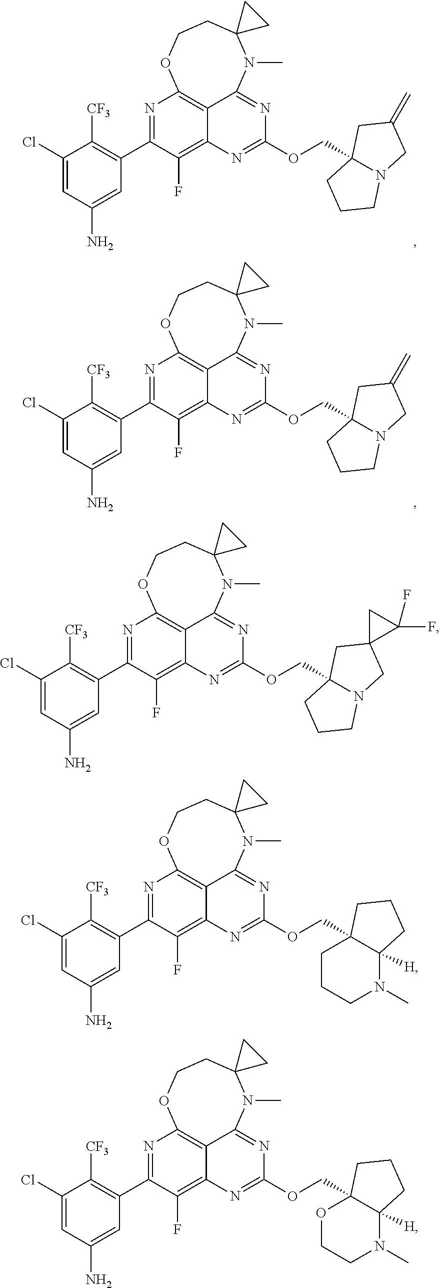

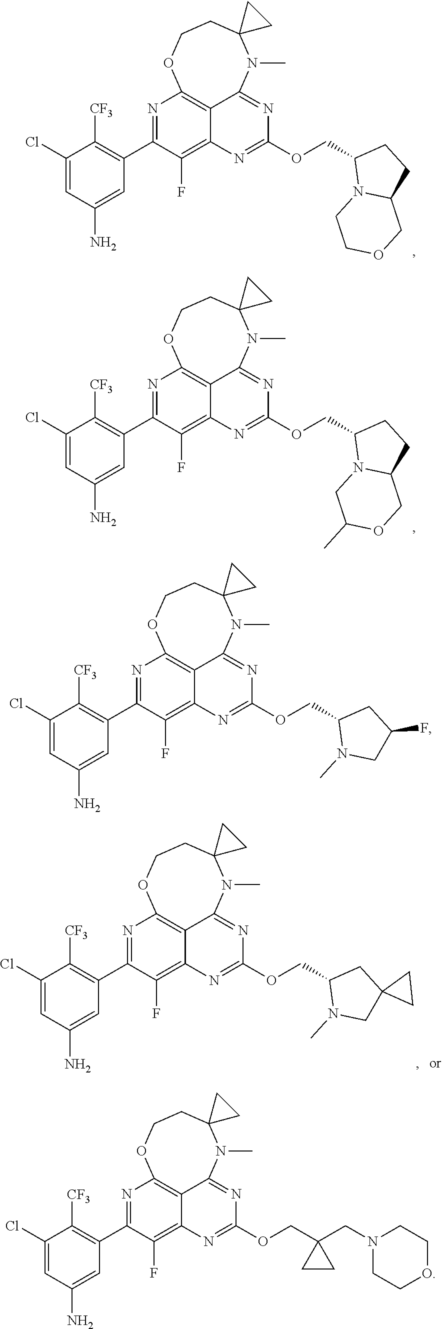 [SMILES: CN1c2nc(OC[C@@H]3CC[C@H]4COCCN43)nc3c(F)c(-c4cc(N)cc(Cl)c4C(F)(F)F)nc(c23)OCCC12CC2 ; CC1CN2[C@H](COc3nc4c5c(nc(-c6cc(N)cc(Cl)c6C(F)(F)F)c(F)c5n3)OCCC3(CC3)N4C)CC[C@H]2CO1 ; CN1C[C@H](F)C[C@H]1COc1nc2c3c(nc(-c4cc(N)cc(Cl)c4C(F)(F)F)c(F)c3n1)OCCC1(CC1)N2C ; CN1CC2(CC2)C[C@H]1COc1nc2c3c(nc(-c4cc(N)cc(Cl)c4C(F)(F)F)c(F)c3n1)OCCC1(CC1)N2C ; CN1c2nc(OCC3(CN4CCOCC4)CC3)nc3c(F)c(-c4cc(N)cc(Cl)c4C(F)(F)F)nc(c23)OCCC12CC2]

Aspect 12: In some embodiments, the compounds provided herein have the following formula:

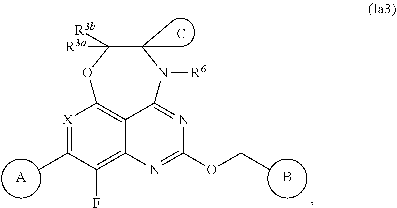

and pharmaceutically acceptable salts, tautomers, stereoisomers, enantiomers, atropisomers, isotopologues, and prodrugs thereof,
wherein:
ring C is unsubstituted or substituted C₃₋₄ cycloalkyl, or unsubstituted or substituted 3-membered to 4-membered heterocyclyl.

In one embodiment, each of R^{3a}, andnR^{3b}, independently, is H, OH, CN, unsubstituted or substituted amino, or unsubstituted or substituted C₁₋₄ alkyl. In one embodiment, each of R^{3a}, and R^{3b}, independently, is H, or unsubstituted or substituted methyl. In one embodiment, each of R^{3a}, and R^{3b}, independently, is H, or unsubstituted methyl. In one embodiment, each of R^{3a}, and R^{3b}, independently, is H.

In one embodiment, ring C is a cyclopropyl ring, a cyclobutyl ring, an oxetane ring, optionally substituted with OH, CN, unsubstituted or substituted amino, or unsubstituted or substituted C₁₋₄ alkyl. In one embodiment, ring C is a cyclopropyl ring, a cyclobutyl ring, an oxetane ring, optionally substituted with OH.

In one embodiment, R⁶ is H, unsubstituted or substituted C₁₋₄ alkyl, unsubstituted or substituted C₃₋₄ cycloalkyl, or unsubstituted or substituted 3-membered to 4-membered heterocyclyl. In one embodiment, R⁶ is methyl, ethyl, 2-(dimethylamino)-ethyl, 2-(methylamino)-ethyl, 1-(methylamino)propan-2-yl, or cyclopropyl.

In one embodiment, X is N. In one embodiment, X is C—Cl.

In one embodiment, ring A is

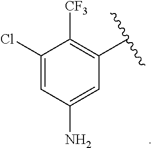 [SMILES: *c1cc(N)cc(Cl)c1C(F)(F)F]

In one embodiment, ring A is

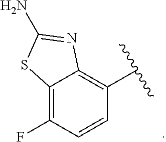 [SMILES: *c1ccc(F)c2sc(N)nc12 ; "."]

In one embodiment, ring A is

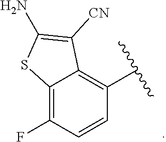 [SMILES: *c1ccc(F)c2sc(N)c(C#N)c12 ; "."]

In one embodiment, ring B is

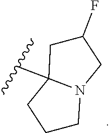 [SMILES: *C12CCCN1CC(F)C2]

In one embodiment, ring B is

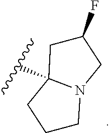 [SMILES: *[C@@]12CCCN1C[C@H](F)C2]

In one embodiment, the compound is

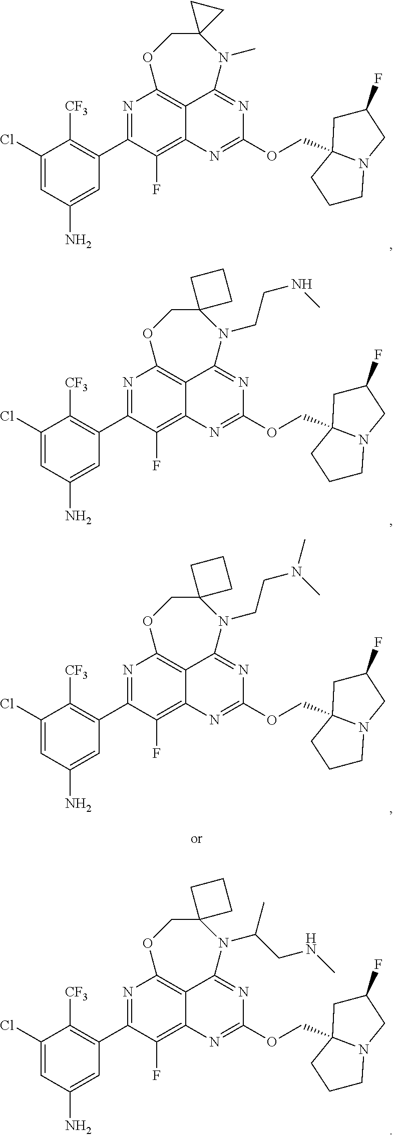 [SMILES: CN1c2nc(OC[C@@]34CCCN3C[C@H](F)C4)nc3c(F)c(-c4cc(N)cc(Cl)c4C(F)(F)F)nc(c23)OCC12CC2 ; CNCCN1c2nc(OC[C@@]34CCCN3C[C@H](F)C4)nc3c(F)c(-c4cc(N)cc(Cl)c4C(F)(F)F)nc(c23)OCC12CCC2 ; CN(C)CCN1c2nc(OC[C@@]34CCCN3C[C@H](F)C4)nc3c(F)c(-c4cc(N)cc(Cl)c4C(F)(F)F)nc(c23)OCC12CCC2 ; CNCC(C)N1c2nc(OC[C@@]34CCCN3C[C@H](F)C4)nc3c(F)c(-c4cc(N)cc(Cl)c4C(F)(F)F)nc(c23)OCC12CCC2]

Aspect 13: Provided herein is a compound selected from the following table:

TABLE 1

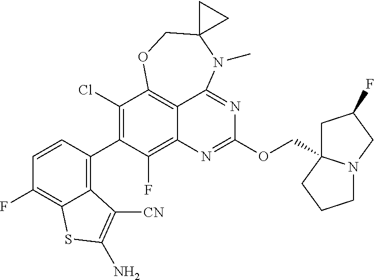 [SMILES: CN1c2nc(OC[C@@]34CCCN3C[C@H](F)C4)nc3c(F)c(-c4ccc(F)c5sc(N)c(C#N)c45)c(Cl)c(c23)OCC12CC2]

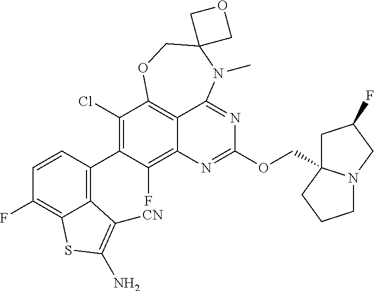 [SMILES: CN1c2nc(OC[C@@]34CCCN3C[C@H](F)C4)nc3c(F)c(-c4ccc(F)c5sc(N)c(C#N)c45)c(Cl)c(c23)OCC12COC2]

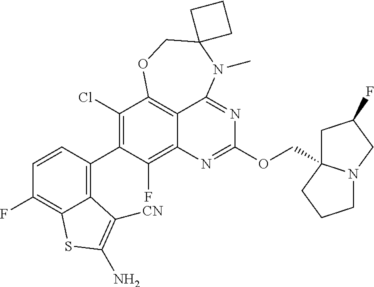 [SMILES: CN1c2nc(OC[C@@]34CCCN3C[C@H](F)C4)nc3c(F)c(-c4ccc(F)c5sc(N)c(C#N)c45)c(Cl)c(c23)OCC12CCC2]

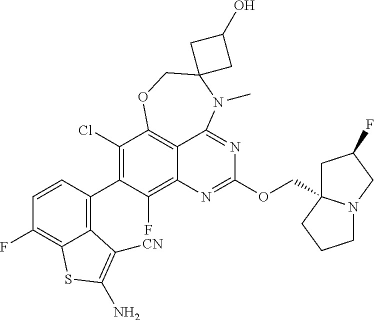 [SMILES: CN1c2nc(OC[C@@]34CCCN3C[C@H](F)C4)nc3c(F)c(-c4ccc(F)c5sc(N)c(C#N)c45)c(Cl)c(c23)OCC12CC(O)C2]

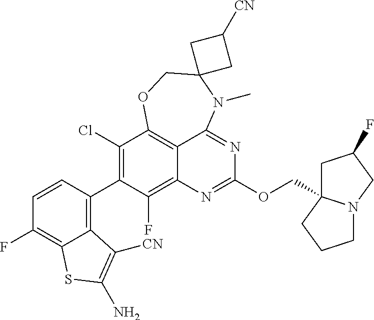 [SMILES: CN1c2nc(OC[C@@]34CCCN3C[C@H](F)C4)nc3c(F)c(-c4ccc(F)c5sc(N)c(C#N)c45)c(Cl)c(c23)OCC12CC(C#N)C2]

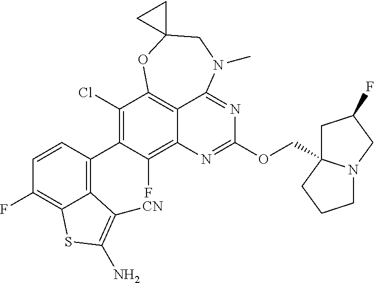 [SMILES: CN1CC2(CC2)Oc2c(Cl)c(-c3ccc(F)c4sc(N)c(C#N)c34)c(F)c3nc(OC[C@@]45CCCN4C[C@H](F)C5)nc1c23]

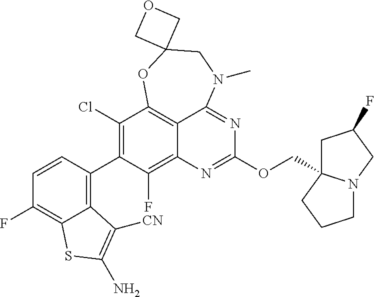 [SMILES: CN1CC2(COC2)Oc2c(Cl)c(-c3ccc(F)c4sc(N)c(C#N)c34)c(F)c3nc(OC[C@@]45CCCN4C[C@H](F)C5)nc1c23]

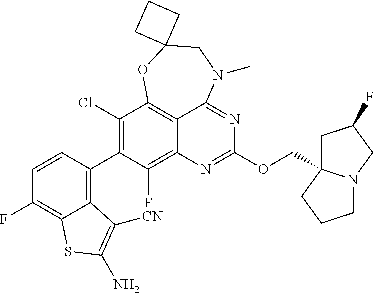 [SMILES: CN1CC2(CCC2)Oc2c(Cl)c(-c3ccc(F)c4sc(N)c(C#N)c34)c(F)c3nc(OC[C@@]45CCCN4C[C@H](F)C5)nc1c23]

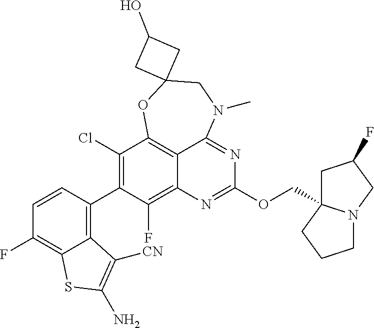 [SMILES: CN1CC2(CC(O)C2)Oc2c(Cl)c(-c3ccc(F)c4sc(N)c(C#N)c34)c(F)c3nc(OC[C@@]45CCCN4C[C@H](F)C5)nc1c23]

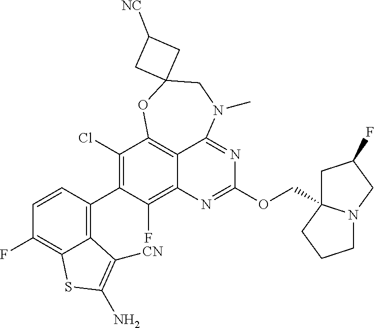 [SMILES: CN1CC2(CC(C#N)C2)Oc2c(Cl)c(-c3ccc(F)c4sc(N)c(C#N)c34)c(F)c3nc(OC[C@@]45CCCN4C[C@H](F)C5)nc1c23]

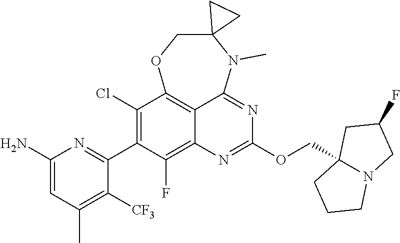 [SMILES: Cc1cc(N)nc(-c2c(Cl)c3c4c(nc(OC[C@@]56CCCN5C[C@H](F)C6)nc4c2F)N(C)C2(CC2)CO3)c1C(F)(F)F]

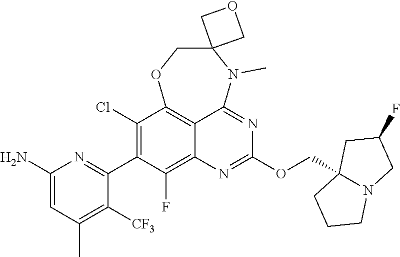 [SMILES: Cc1cc(N)nc(-c2c(Cl)c3c4c(nc(OC[C@@]56CCCN5C[C@H](F)C6)nc4c2F)N(C)C2(COC2)CO3)c1C(F)(F)F]

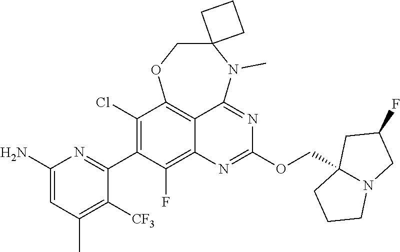 [SMILES: Cc1cc(N)nc(-c2c(Cl)c3c4c(nc(OC[C@@]56CCCN5C[C@H](F)C6)nc4c2F)N(C)C2(CCC2)CO3)c1C(F)(F)F]

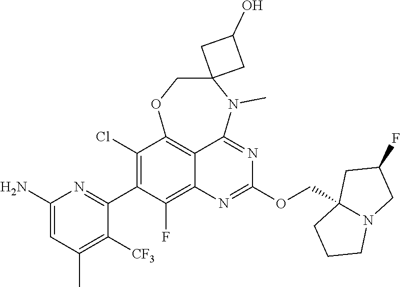 [SMILES: Cc1cc(N)nc(-c2c(Cl)c3c4c(nc(OC[C@@]56CCCN5C[C@H](F)C6)nc4c2F)N(C)C2(CO3)CC(O)C2)c1C(F)(F)F]

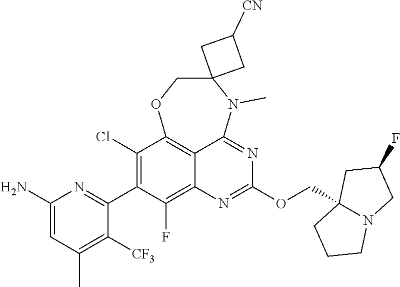 [SMILES: Cc1cc(N)nc(-c2c(Cl)c3c4c(nc(OC[C@@]56CCCN5C[C@H](F)C6)nc4c2F)N(C)C2(CO3)CC(C#N)C2)c1C(F)(F)F]

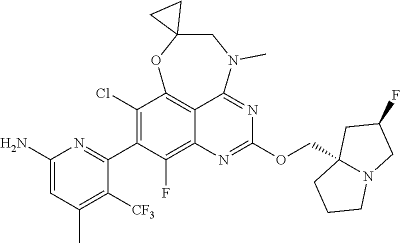 [SMILES: Cc1cc(N)nc(-c2c(Cl)c3c4c(nc(OC[C@@]56CCCN5C[C@H](F)C6)nc4c2F)N(C)CC2(CC2)O3)c1C(F)(F)F]

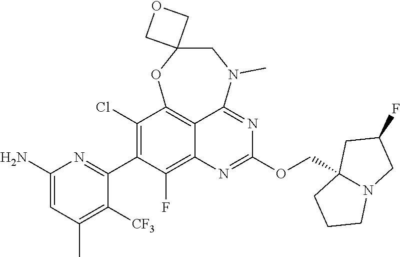 [SMILES: Cc1cc(N)nc(-c2c(Cl)c3c4c(nc(OC[C@@]56CCCN5C[C@H](F)C6)nc4c2F)N(C)CC2(COC2)O3)c1C(F)(F)F]

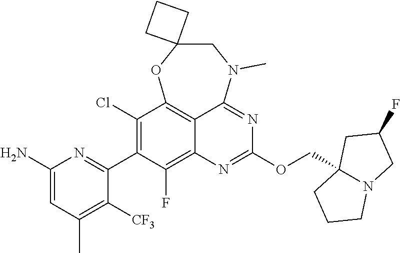 [SMILES: Cc1cc(N)nc(-c2c(Cl)c3c4c(nc(OC[C@@]56CCCN5C[C@H](F)C6)nc4c2F)N(C)CC2(CCC2)O3)c1C(F)(F)F]

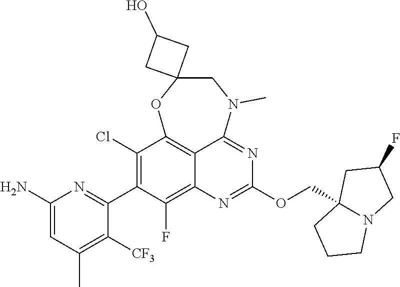 [SMILES: Cc1cc(N)nc(-c2c(Cl)c3c4c(nc(OC[C@@]56CCCN5C[C@H](F)C6)nc4c2F)N(C)CC2(CC(O)C2)O3)c1C(F)(F)F]

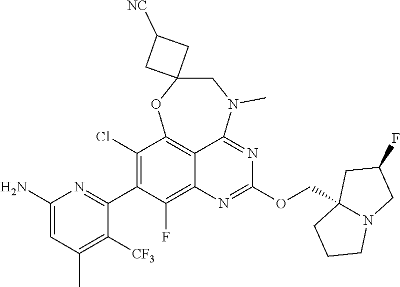 [SMILES: Cc1cc(N)nc(-c2c(Cl)c3c4c(nc(OC[C@@]56CCCN5C[C@H](F)C6)nc4c2F)N(C)CC2(CC(C#N)C2)O3)c1C(F)(F)F]

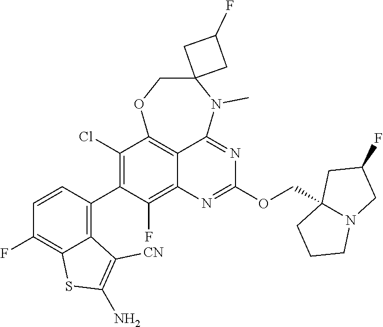 [SMILES: CN1c2nc(OC[C@@]34CCCN3C[C@H](F)C4)nc3c(F)c(-c4ccc(F)c5sc(N)c(C#N)c45)c(Cl)c(c23)OCC12CC(F)C2]

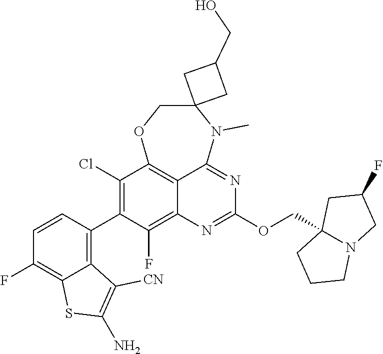 [SMILES: CN1c2nc(OC[C@@]34CCCN3C[C@H](F)C4)nc3c(F)c(-c4ccc(F)c5sc(N)c(C#N)c45)c(Cl)c(c23)OCC12CC(CO)C2]

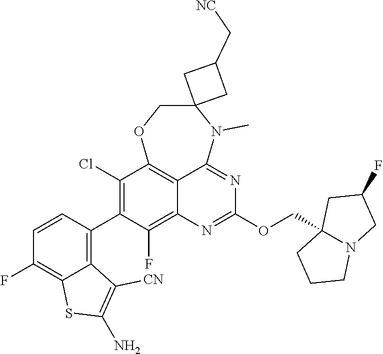 [SMILES: CN1c2nc(OC[C@@]34CCCN3C[C@H](F)C4)nc3c(F)c(-c4ccc(F)c5sc(N)c(C#N)c45)c(Cl)c(c23)OCC12CC(CC#N)C2]

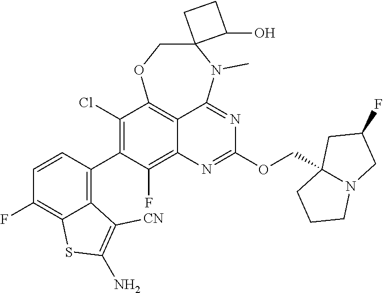 [SMILES: CN1c2nc(OC[C@@]34CCCN3C[C@H](F)C4)nc3c(F)c(-c4ccc(F)c5sc(N)c(C#N)c45)c(Cl)c(c23)OCC12CCC2O]

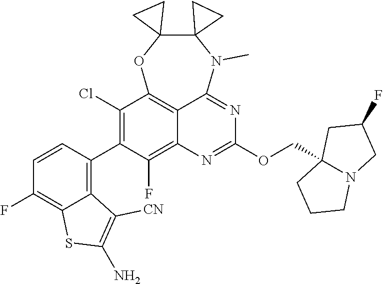 [SMILES: CN1c2nc(OC[C@@]34CCCN3C[C@H](F)C4)nc3c(F)c(-c4ccc(F)c5sc(N)c(C#N)c45)c(Cl)c(c23)OC2(CC2)C12CC2]

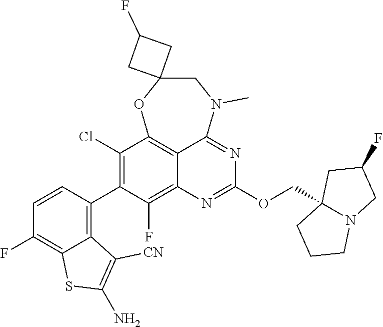 [SMILES: CN1CC2(CC(F)C2)Oc2c(Cl)c(-c3ccc(F)c4sc(N)c(C#N)c34)c(F)c3nc(OC[C@@]45CCCN4C[C@H](F)C5)nc1c23]

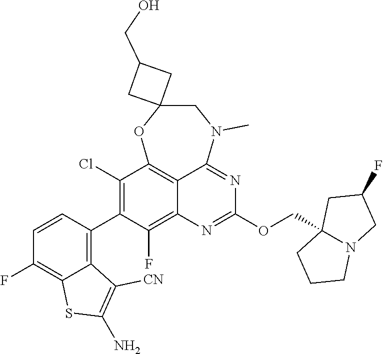 [SMILES: CN1CC2(CC(CO)C2)Oc2c(Cl)c(-c3ccc(F)c4sc(N)c(C#N)c34)c(F)c3nc(OC[C@@]45CCCN4C[C@H](F)C5)nc1c23]

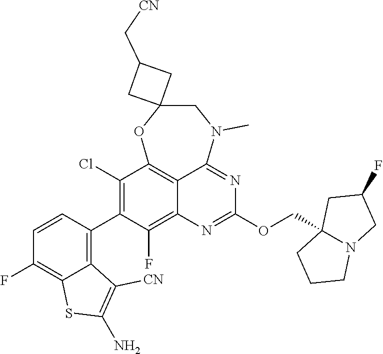 [SMILES: CN1CC2(CC(CC#N)C2)Oc2c(Cl)c(-c3ccc(F)c4sc(N)c(C#N)c34)c(F)c3nc(OC[C@@]45CCCN4C[C@H](F)C5)nc1c23]

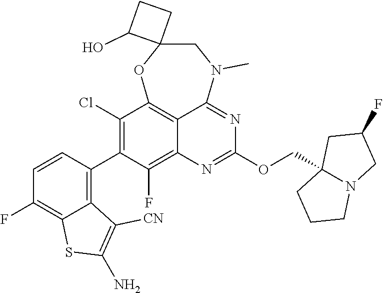 [SMILES: CN1CC2(CCC2O)Oc2c(Cl)c(-c3ccc(F)c4sc(N)c(C#N)c34)c(F)c3nc(OC[C@@]45CCCN4C[C@H](F)C5)nc1c23]

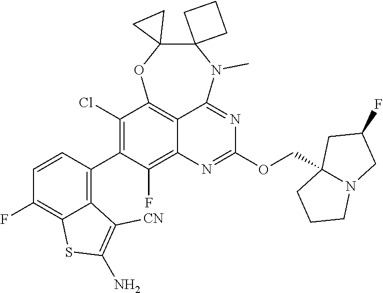 [SMILES: CN1c2nc(OC[C@@]34CCCN3C[C@H](F)C4)nc3c(F)c(-c4ccc(F)c5sc(N)c(C#N)c45)c(Cl)c(c23)OC2(CC2)C12CCC2]

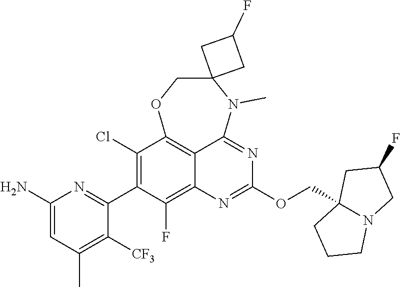 [SMILES: Cc1cc(N)nc(-c2c(Cl)c3c4c(nc(OC[C@@]56CCCN5C[C@H](F)C6)nc4c2F)N(C)C2(CO3)CC(F)C2)c1C(F)(F)F]

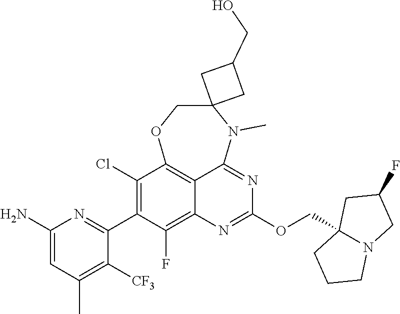 [SMILES: Cc1cc(N)nc(-c2c(Cl)c3c4c(nc(OC[C@@]56CCCN5C[C@H](F)C6)nc4c2F)N(C)C2(CO3)CC(CO)C2)c1C(F)(F)F]

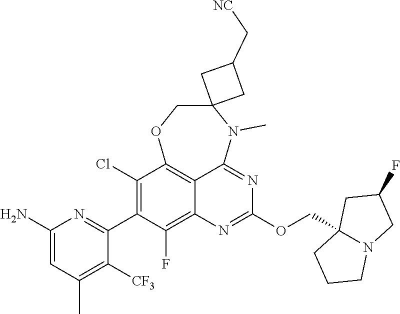 [SMILES: Cc1cc(N)nc(-c2c(Cl)c3c4c(nc(OC[C@@]56CCCN5C[C@H](F)C6)nc4c2F)N(C)C2(CO3)CC(CC#N)C2)c1C(F)(F)F]

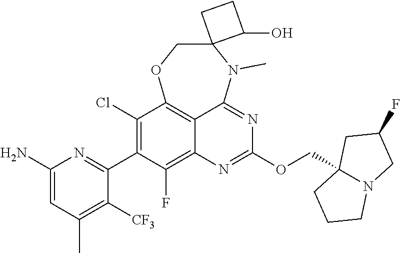 [SMILES: Cc1cc(N)nc(-c2c(Cl)c3c4c(nc(OC[C@@]56CCCN5C[C@H](F)C6)nc4c2F)N(C)C2(CCC2O)CO3)c1C(F)(F)F]

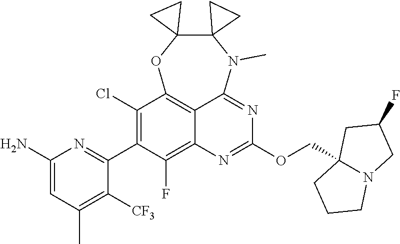 [SMILES: Cc1cc(N)nc(-c2c(Cl)c3c4c(nc(OC[C@@]56CCCN5C[C@H](F)C6)nc4c2F)N(C)C2(CC2)C2(CC2)O3)c1C(F)(F)F]

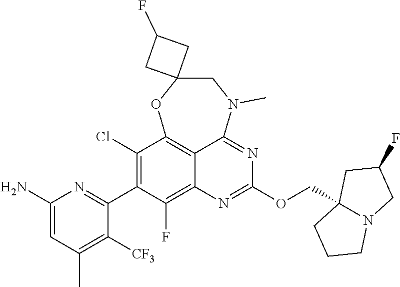 [SMILES: Cc1cc(N)nc(-c2c(Cl)c3c4c(nc(OC[C@@]56CCCN5C[C@H](F)C6)nc4c2F)N(C)CC2(CC(F)C2)O3)c1C(F)(F)F]

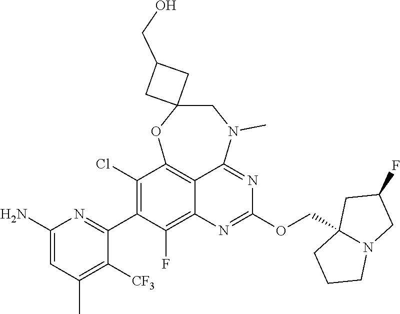 [SMILES: Cc1cc(N)nc(-c2c(Cl)c3c4c(nc(OC[C@@]56CCCN5C[C@H](F)C6)nc4c2F)N(C)CC2(CC(CO)C2)O3)c1C(F)(F)F]

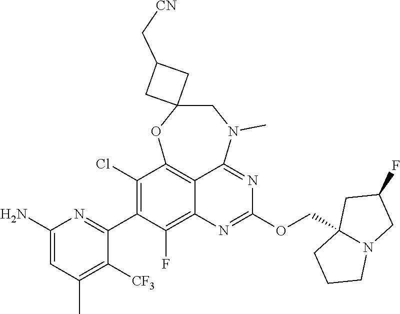 [SMILES: Cc1cc(N)nc(-c2c(Cl)c3c4c(nc(OC[C@@]56CCCN5C[C@H](F)C6)nc4c2F)N(C)CC2(CC(CC#N)C2)O3)c1C(F)(F)F]

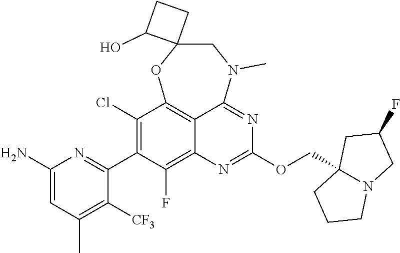 [SMILES: Cc1cc(N)nc(-c2c(Cl)c3c4c(nc(OC[C@@]56CCCN5C[C@H](F)C6)nc4c2F)N(C)CC2(CCC2O)O3)c1C(F)(F)F]

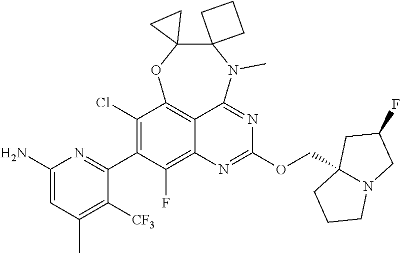 [SMILES: Cc1cc(N)nc(-c2c(Cl)c3c4c(nc(OC[C@@]56CCCN5C[C@H](F)C6)nc4c2F)N(C)C2(CCC2)C2(CC2)O3)c1C(F)(F)F]

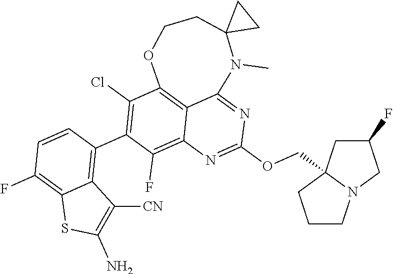 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CC2]

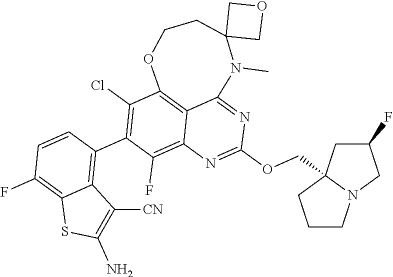 [SMILES: CN1c2nc(OC[C@@]34CCCN3C[C@H](F)C4)nc3c(F)c(-c4ccc(F)c5sc(N)c(C#N)c45)c(Cl)c(c23)OCCC12COC2]

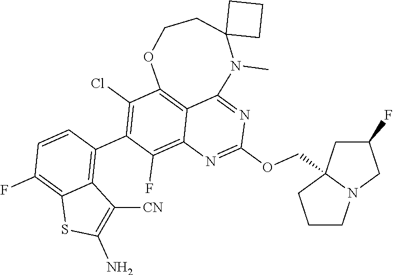 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CCC2]

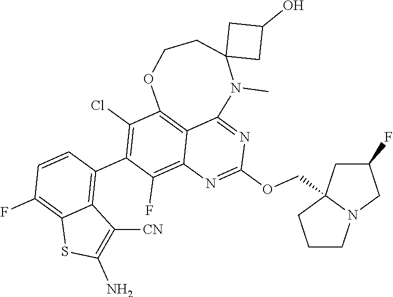 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CC(O)C2]

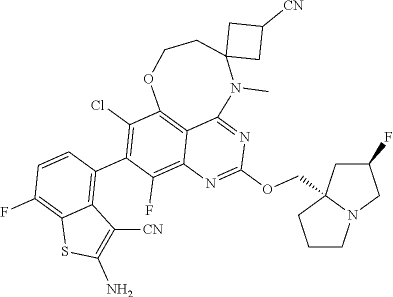 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CC(C#N)C2]

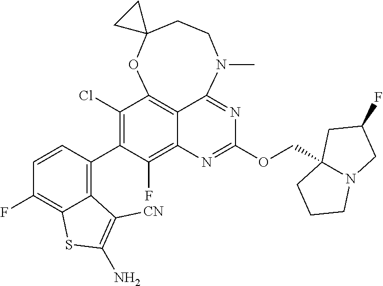 [SMILES: CN1CCC2(CC2)Oc2c(Cl)c(-c3ccc(F)c4sc(N)c(C#N)c34)c(F)c3nc(OC[C@@]45CCCN4C[C@H](F)C5)nc1c23]

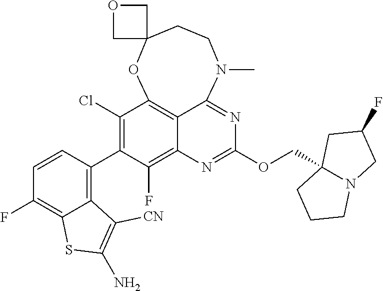 [SMILES: CN1CCC2(COC2)Oc2c(Cl)c(-c3ccc(F)c4sc(N)c(C#N)c34)c(F)c3nc(OC[C@@]45CCCN4C[C@H](F)C5)nc1c23]

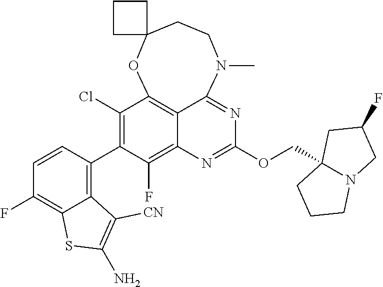 [SMILES: CN1CCC2(CCC2)Oc2c(Cl)c(-c3ccc(F)c4sc(N)c(C#N)c34)c(F)c3nc(OC[C@@]45CCCN4C[C@H](F)C5)nc1c23]

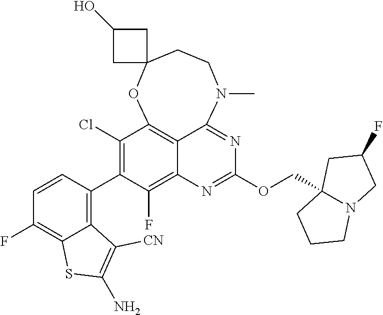 [SMILES: CN1CCC2(CC(O)C2)Oc2c(Cl)c(-c3ccc(F)c4sc(N)c(C#N)c34)c(F)c3nc(OC[C@@]45CCCN4C[C@H](F)C5)nc1c23]

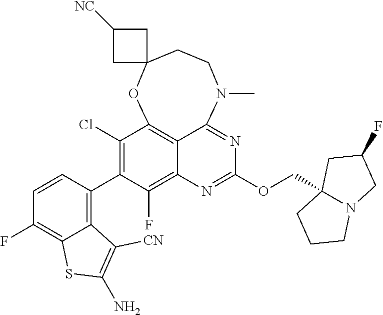 [SMILES: CN1CCC2(CC(C#N)C2)Oc2c(Cl)c(-c3ccc(F)c4sc(N)c(C#N)c34)c(F)c3nc(OC[C@@]45CCCN4C[C@H](F)C5)nc1c23]

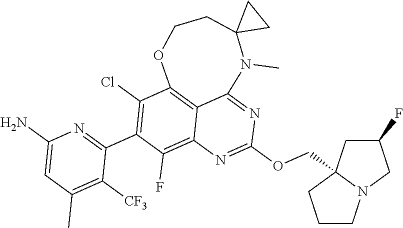 [SMILES: Cc1cc(N)nc(-c2c(Cl)c3c4c(nc(OC[C@@]56CCCN5C[C@H](F)C6)nc4c2F)N(C)C2(CCO3)CC2)c1C(F)(F)F]

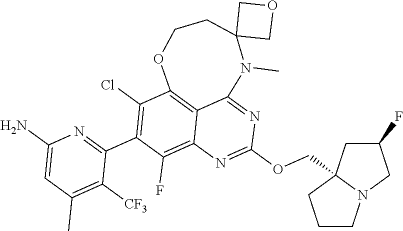 [SMILES: Cc1cc(N)nc(-c2c(Cl)c3c4c(nc(OC[C@@]56CCCN5C[C@H](F)C6)nc4c2F)N(C)C2(CCO3)COC2)c1C(F)(F)F]

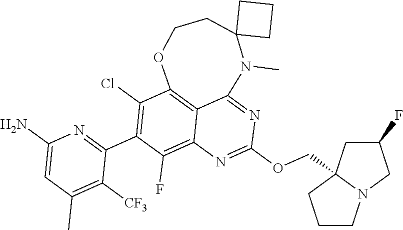 [SMILES: Cc1cc(N)nc(-c2c(Cl)c3c4c(nc(OC[C@@]56CCCN5C[C@H](F)C6)nc4c2F)N(C)C2(CCC2)CCO3)c1C(F)(F)F]

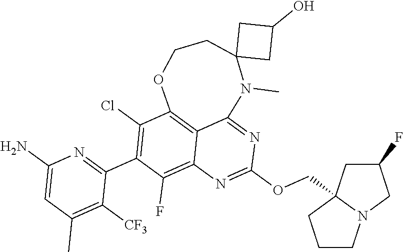 [SMILES: Cc1cc(N)nc(-c2c(Cl)c3c4c(nc(OC[C@@]56CCCN5C[C@H](F)C6)nc4c2F)N(C)C2(CCO3)CC(O)C2)c1C(F)(F)F]

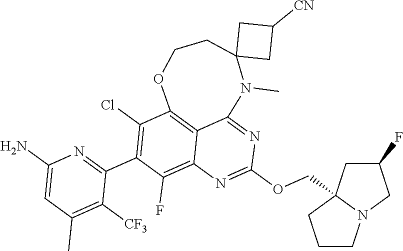 [SMILES: Cc1cc(N)nc(-c2c(Cl)c3c4c(nc(OC[C@@]56CCCN5C[C@H](F)C6)nc4c2F)N(C)C2(CCO3)CC(C#N)C2)c1C(F)(F)F]

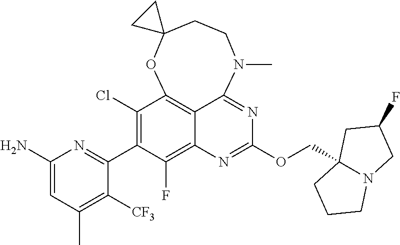 [SMILES: Cc1cc(N)nc(-c2c(Cl)c3c4c(nc(OC[C@@]56CCCN5C[C@H](F)C6)nc4c2F)N(C)CCC2(CC2)O3)c1C(F)(F)F]

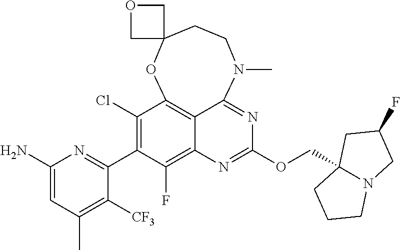 [SMILES: Cc1cc(N)nc(-c2c(Cl)c3c4c(nc(OC[C@@]56CCCN5C[C@H](F)C6)nc4c2F)N(C)CCC2(COC2)O3)c1C(F)(F)F]

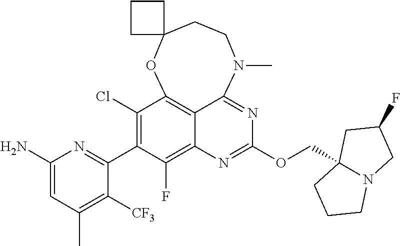 [SMILES: Cc1cc(N)nc(-c2c(Cl)c3c4c(nc(OC[C@@]56CCCN5C[C@H](F)C6)nc4c2F)N(C)CCC2(CCC2)O3)c1C(F)(F)F]

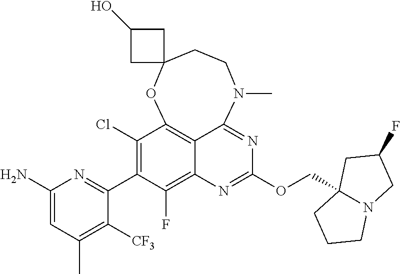 [SMILES: Cc1cc(N)nc(-c2c(Cl)c3c4c(nc(OC[C@@]56CCCN5C[C@H](F)C6)nc4c2F)N(C)CCC2(CC(O)C2)O3)c1C(F)(F)F]

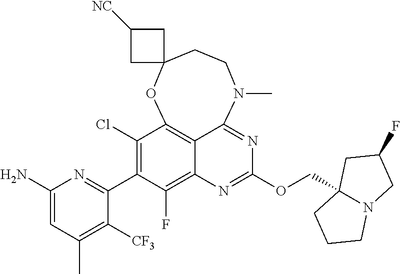 [SMILES: Cc1cc(N)nc(-c2c(Cl)c3c4c(nc(OC[C@@]56CCCN5C[C@H](F)C6)nc4c2F)N(C)CCC2(CC(C#N)C2)O3)c1C(F)(F)F]

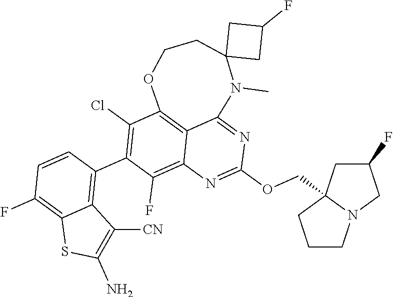 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CC(F)C2]

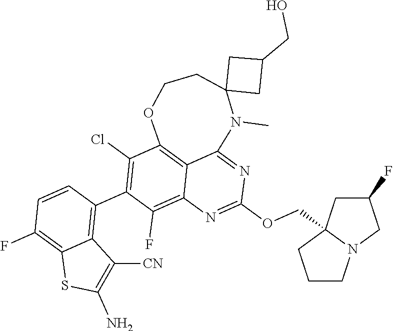 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CC(CO)C2]

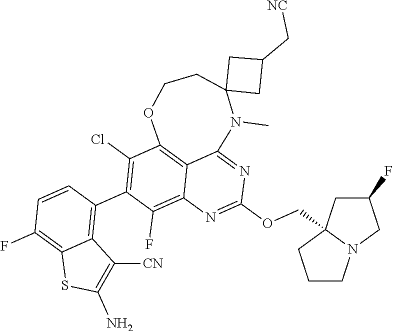 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CC(CC#N)C2]

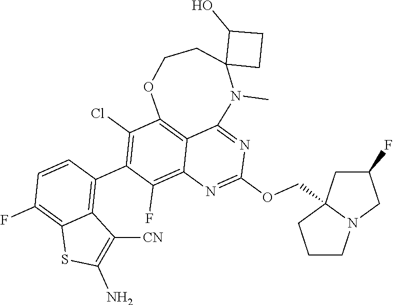 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CCC2O]

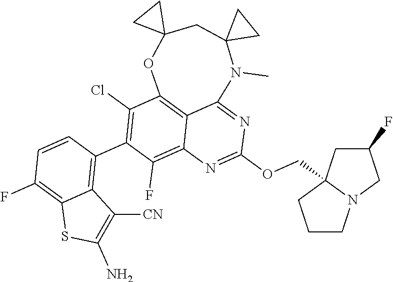 [SMILES: CN1c2nc(OC[C@@]34CCCN3C[C@H](F)C4)nc3c(F)c(-c4ccc(F)c5sc(N)c(C#N)c45)c(Cl)c(c23)OC2(CC2)CC12CC2]

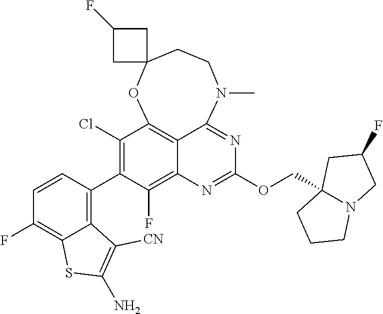 [SMILES: CN1CCC2(CC(F)C2)Oc2c(Cl)c(-c3ccc(F)c4sc(N)c(C#N)c34)c(F)c3nc(OC[C@@]45CCCN4C[C@H](F)C5)nc1c23]

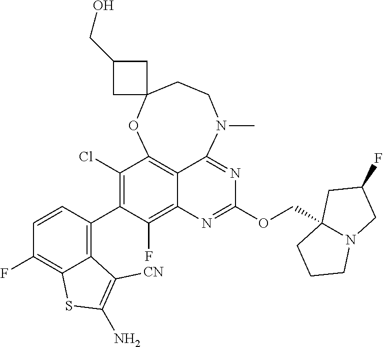 [SMILES: CN1CCC2(CC(CO)C2)Oc2c(Cl)c(-c3ccc(F)c4sc(N)c(C#N)c34)c(F)c3nc(OC[C@@]45CCCN4C[C@H](F)C5)nc1c23]

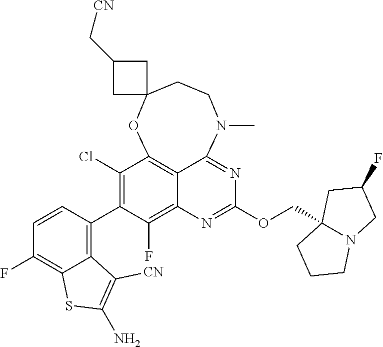 [SMILES: CN1CCC2(CC(CC#N)C2)Oc2c(Cl)c(-c3ccc(F)c4sc(N)c(C#N)c34)c(F)c3nc(OC[C@@]45CCCN4C[C@H](F)C5)nc1c23]

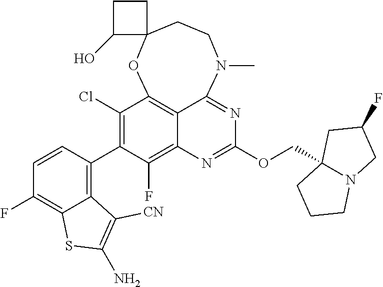 [SMILES: CN1CCC2(CCC2O)Oc2c(Cl)c(-c3ccc(F)c4sc(N)c(C#N)c34)c(F)c3nc(OC[C@@]45CCCN4C[C@H](F)C5)nc1c23]

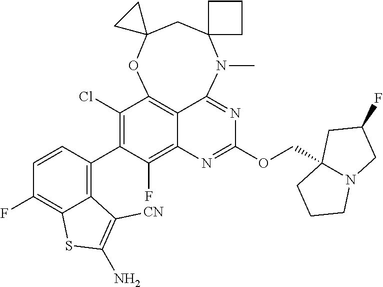 [SMILES: CN1c2nc(OC[C@@]34CCCN3C[C@H](F)C4)nc3c(F)c(-c4ccc(F)c5sc(N)c(C#N)c45)c(Cl)c(c23)OC2(CC2)CC12CCC2]

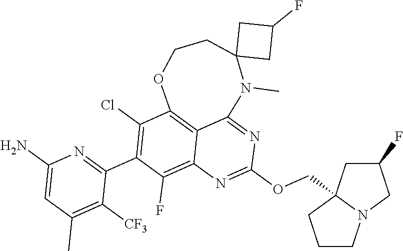 [SMILES: Cc1cc(N)nc(-c2c(Cl)c3c4c(nc(OC[C@@]56CCCN5C[C@H](F)C6)nc4c2F)N(C)C2(CCO3)CC(F)C2)c1C(F)(F)F]

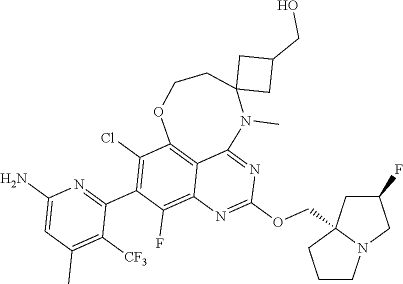 [SMILES: Cc1cc(N)nc(-c2c(Cl)c3c4c(nc(OC[C@@]56CCCN5C[C@H](F)C6)nc4c2F)N(C)C2(CCO3)CC(CO)C2)c1C(F)(F)F]

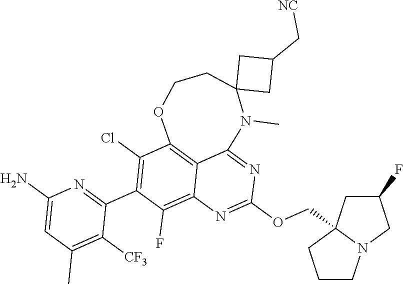 [SMILES: Cc1cc(N)nc(-c2c(Cl)c3c4c(nc(OC[C@@]56CCCN5C[C@H](F)C6)nc4c2F)N(C)C2(CCO3)CC(CC#N)C2)c1C(F)(F)F]

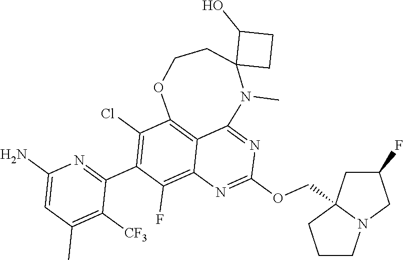 [SMILES: Cc1cc(N)nc(-c2c(Cl)c3c4c(nc(OC[C@@]56CCCN5C[C@H](F)C6)nc4c2F)N(C)C2(CCO3)CCC2O)c1C(F)(F)F]

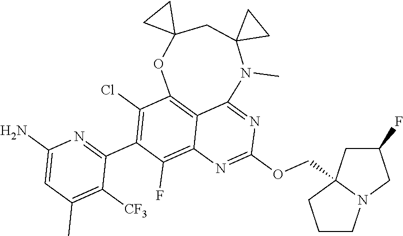 [SMILES: Cc1cc(N)nc(-c2c(Cl)c3c4c(nc(OC[C@@]56CCCN5C[C@H](F)C6)nc4c2F)N(C)C2(CC2)CC2(CC2)O3)c1C(F)(F)F]

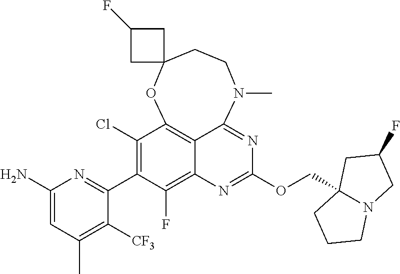 [SMILES: Cc1cc(N)nc(-c2c(Cl)c3c4c(nc(OC[C@@]56CCCN5C[C@H](F)C6)nc4c2F)N(C)CCC2(CC(F)C2)O3)c1C(F)(F)F]

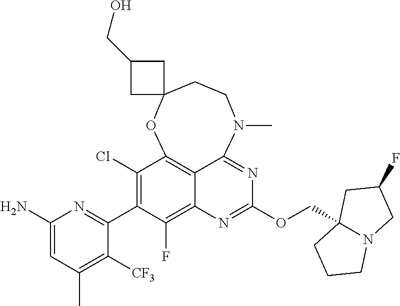 [SMILES: Cc1cc(N)nc(-c2c(Cl)c3c4c(nc(OC[C@@]56CCCN5C[C@H](F)C6)nc4c2F)N(C)CCC2(CC(CO)C2)O3)c1C(F)(F)F]

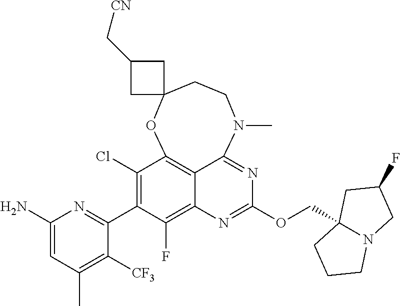 [SMILES: Cc1cc(N)nc(-c2c(Cl)c3c4c(nc(OC[C@@]56CCCN5C[C@H](F)C6)nc4c2F)N(C)CCC2(CC(CC#N)C2)O3)c1C(F)(F)F]

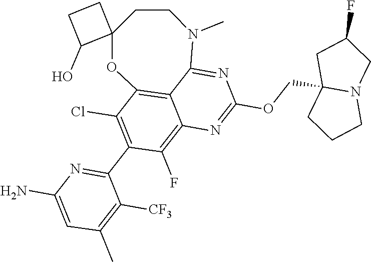 [SMILES: Cc1cc(N)nc(-c2c(Cl)c3c4c(nc(OC[C@@]56CCCN5C[C@H](F)C6)nc4c2F)N(C)CCC2(CCC2O)O3)c1C(F)(F)F]

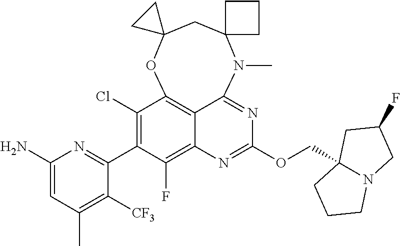 [SMILES: Cc1cc(N)nc(-c2c(Cl)c3c4c(nc(OC[C@@]56CCCN5C[C@H](F)C6)nc4c2F)N(C)C2(CCC2)CC2(CC2)O3)c1C(F)(F)F]

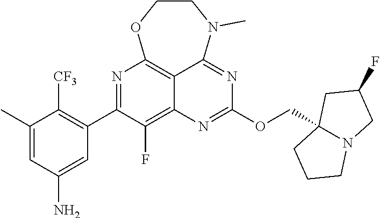 [SMILES: Cc1cc(N)cc(-c2nc3c4c(nc(OC[C@@]56CCCN5C[C@H](F)C6)nc4c2F)N(C)CCO3)c1C(F)(F)F]

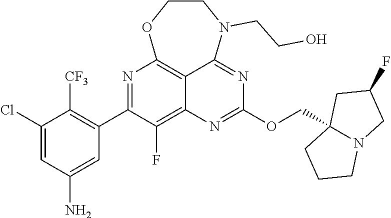 [SMILES: Nc1cc(Cl)c(C(F)(F)F)c(-c2nc3c4c(nc(OC[C@@]56CCCN5C[C@H](F)C6)nc4c2F)N(CCO)CCO3)c1]

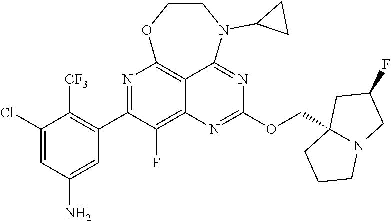 [SMILES: Nc1cc(Cl)c(C(F)(F)F)c(-c2nc3c4c(nc(OC[C@@]56CCCN5C[C@H](F)C6)nc4c2F)N(C2CC2)CCO3)c1]

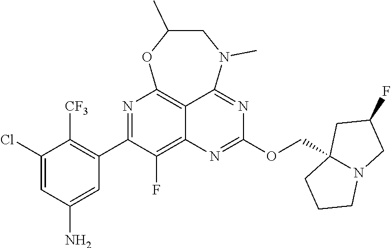 [SMILES: CC1CN(C)c2nc(OC[C@@]34CCCN3C[C@H](F)C4)nc3c(F)c(-c4cc(N)cc(Cl)c4C(F)(F)F)nc(c23)O1]

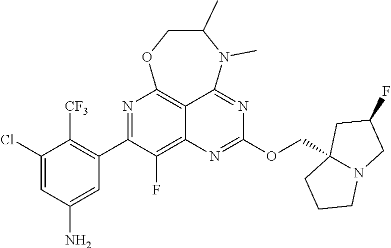 [SMILES: CC1COc2nc(-c3cc(N)cc(Cl)c3C(F)(F)F)c(F)c3nc(OC[C@@]45CCCN4C[C@H](F)C5)nc(c23)N1C]

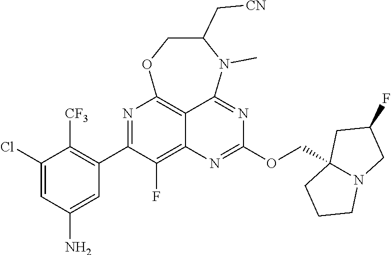 [SMILES: CN1c2nc(OC[C@@]34CCCN3C[C@H](F)C4)nc3c(F)c(-c4cc(N)cc(Cl)c4C(F)(F)F)nc(c23)OCC1CC#N]

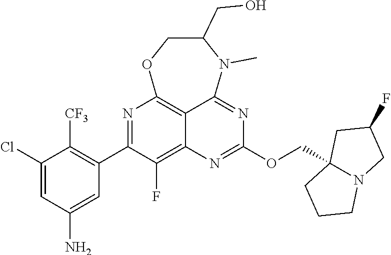 [SMILES: CN1c2nc(OC[C@@]34CCCN3C[C@H](F)C4)nc3c(F)c(-c4cc(N)cc(Cl)c4C(F)(F)F)nc(c23)OCC1CO]

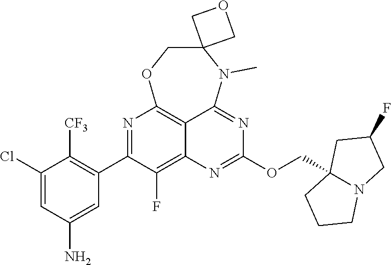 [SMILES: CN1c2nc(OC[C@@]34CCCN3C[C@H](F)C4)nc3c(F)c(-c4cc(N)cc(Cl)c4C(F)(F)F)nc(c23)OCC12COC2]

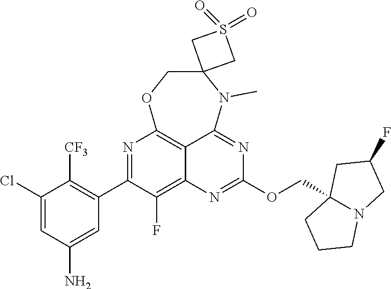 [SMILES: CN1c2nc(OC[C@@]34CCCN3C[C@H](F)C4)nc3c(F)c(-c4cc(N)cc(Cl)c4C(F)(F)F)nc(c23)OCC12CS(=O)(=O)C2]

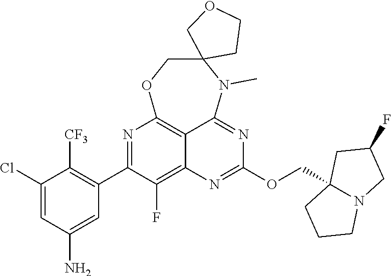 [SMILES: CN1c2nc(OC[C@@]34CCCN3C[C@H](F)C4)nc3c(F)c(-c4cc(N)cc(Cl)c4C(F)(F)F)nc(c23)OCC12CCOC2]

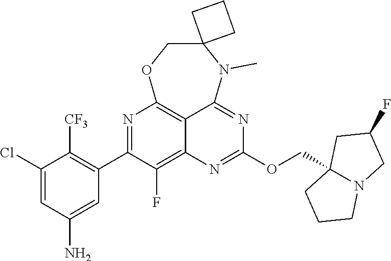 [SMILES: CN1c2nc(OC[C@@]34CCCN3C[C@H](F)C4)nc3c(F)c(-c4cc(N)cc(Cl)c4C(F)(F)F)nc(c23)OCC12CCC2]

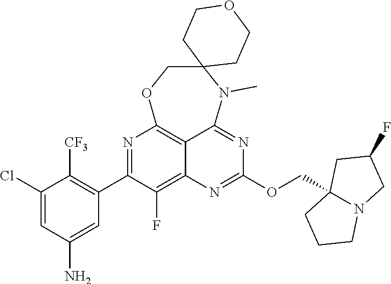 [SMILES: CN1c2nc(OC[C@@]34CCCN3C[C@H](F)C4)nc3c(F)c(-c4cc(N)cc(Cl)c4C(F)(F)F)nc(c23)OCC12CCOCC2]

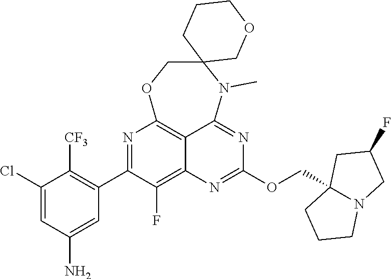 [SMILES: CN1c2nc(OC[C@@]34CCCN3C[C@H](F)C4)nc3c(F)c(-c4cc(N)cc(Cl)c4C(F)(F)F)nc(c23)OCC12CCCOC2]

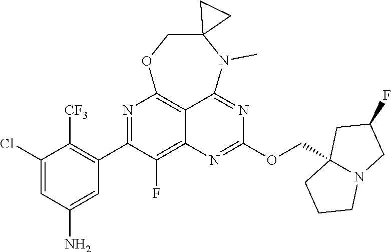 [SMILES: CN1c2nc(OC[C@@]34CCCN3C[C@H](F)C4)nc3c(F)c(-c4cc(N)cc(Cl)c4C(F)(F)F)nc(c23)OCC12CC2]

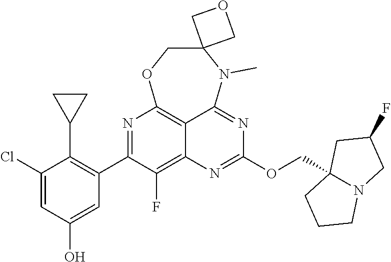 [SMILES: CN1c2nc(OC[C@@]34CCCN3C[C@H](F)C4)nc3c(F)c(-c4cc(O)cc(Cl)c4C4CC4)nc(c23)OCC12COC2]

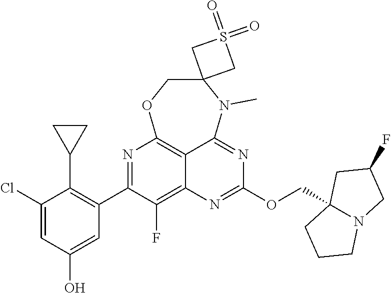 [SMILES: CN1c2nc(OC[C@@]34CCCN3C[C@H](F)C4)nc3c(F)c(-c4cc(O)cc(Cl)c4C4CC4)nc(c23)OCC12CS(=O)(=O)C2]

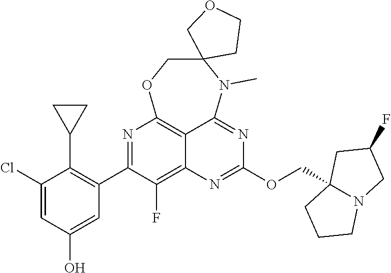 [SMILES: CN1c2nc(OC[C@@]34CCCN3C[C@H](F)C4)nc3c(F)c(-c4cc(O)cc(Cl)c4C4CC4)nc(c23)OCC12CCOC2]

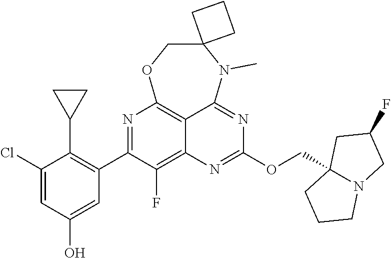 [SMILES: CN1c2nc(OC[C@@]34CCCN3C[C@H](F)C4)nc3c(F)c(-c4cc(O)cc(Cl)c4C4CC4)nc(c23)OCC12CCC2]

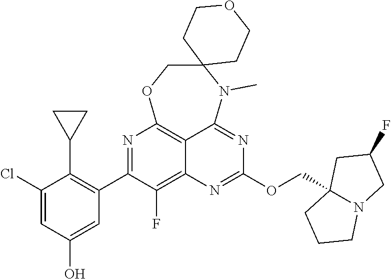 [SMILES: CN1c2nc(OC[C@@]34CCCN3C[C@H](F)C4)nc3c(F)c(-c4cc(O)cc(Cl)c4C4CC4)nc(c23)OCC12CCOCC2]

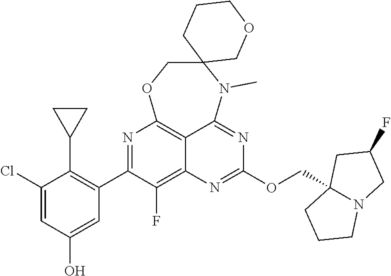 [SMILES: CN1c2nc(OC[C@@]34CCCN3C[C@H](F)C4)nc3c(F)c(-c4cc(O)cc(Cl)c4C4CC4)nc(c23)OCC12CCCOC2]

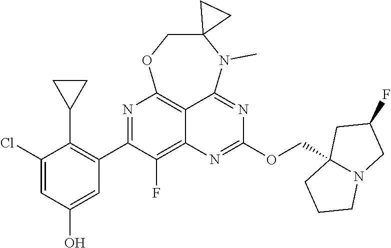 [SMILES: CN1c2nc(OC[C@@]34CCCN3C[C@H](F)C4)nc3c(F)c(-c4cc(O)cc(Cl)c4C4CC4)nc(c23)OCC12CC2]

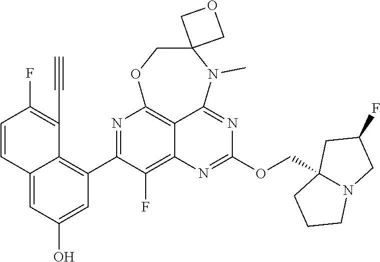 [SMILES: C#Cc1c(F)ccc2cc(O)cc(-c3nc4c5c(nc(OC[C@@]67CCCN6C[C@H](F)C7)nc5c3F)N(C)C3(COC3)CO4)c12]

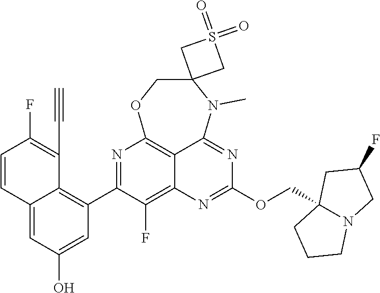 [SMILES: C#Cc1c(F)ccc2cc(O)cc(-c3nc4c5c(nc(OC[C@@]67CCCN6C[C@H](F)C7)nc5c3F)N(C)C3(CO4)CS(=O)(=O)C3)c12]

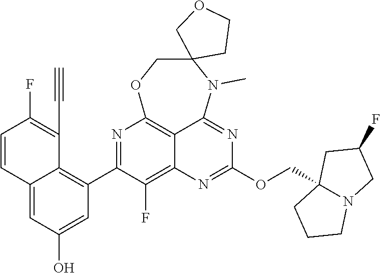 [SMILES: C#Cc1c(F)ccc2cc(O)cc(-c3nc4c5c(nc(OC[C@@]67CCCN6C[C@H](F)C7)nc5c3F)N(C)C3(CCOC3)CO4)c12]

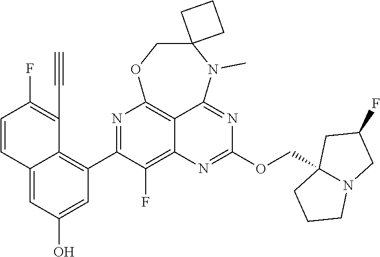 [SMILES: C#Cc1c(F)ccc2cc(O)cc(-c3nc4c5c(nc(OC[C@@]67CCCN6C[C@H](F)C7)nc5c3F)N(C)C3(CCC3)CO4)c12]

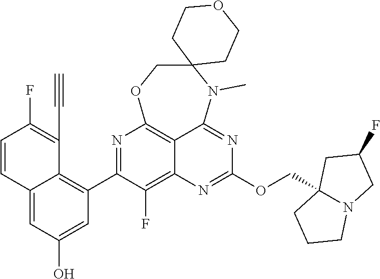 [SMILES: C#Cc1c(F)ccc2cc(O)cc(-c3nc4c5c(nc(OC[C@@]67CCCN6C[C@H](F)C7)nc5c3F)N(C)C3(CCOCC3)CO4)c12]

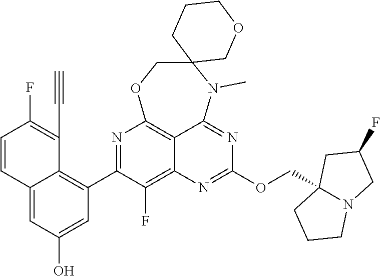 [SMILES: C#Cc1c(F)ccc2cc(O)cc(-c3nc4c5c(nc(OC[C@@]67CCCN6C[C@H](F)C7)nc5c3F)N(C)C3(CCCOC3)CO4)c12]

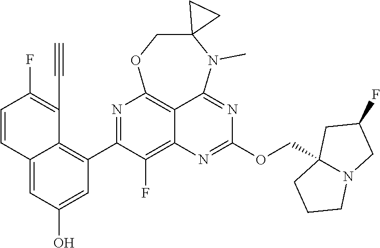 [SMILES: C#Cc1c(F)ccc2cc(O)cc(-c3nc4c5c(nc(OC[C@@]67CCCN6C[C@H](F)C7)nc5c3F)N(C)C3(CC3)CO4)c12]

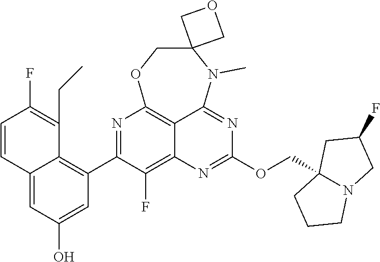 [SMILES: CCc1c(F)ccc2cc(O)cc(-c3nc4c5c(nc(OC[C@@]67CCCN6C[C@H](F)C7)nc5c3F)N(C)C3(COC3)CO4)c12]

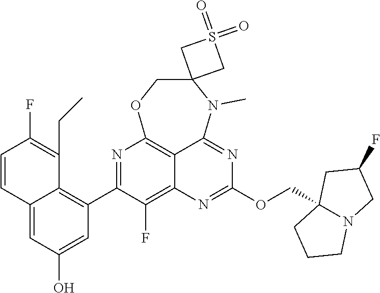 [SMILES: CCc1c(F)ccc2cc(O)cc(-c3nc4c5c(nc(OC[C@@]67CCCN6C[C@H](F)C7)nc5c3F)N(C)C3(CO4)CS(=O)(=O)C3)c12]

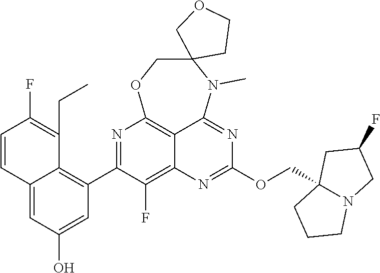 [SMILES: CCc1c(F)ccc2cc(O)cc(-c3nc4c5c(nc(OC[C@@]67CCCN6C[C@H](F)C7)nc5c3F)N(C)C3(CCOC3)CO4)c12]

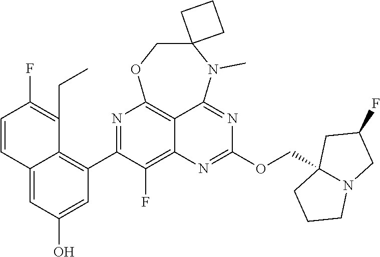 [SMILES: CCc1c(F)ccc2cc(O)cc(-c3nc4c5c(nc(OC[C@@]67CCCN6C[C@H](F)C7)nc5c3F)N(C)C3(CCC3)CO4)c12]

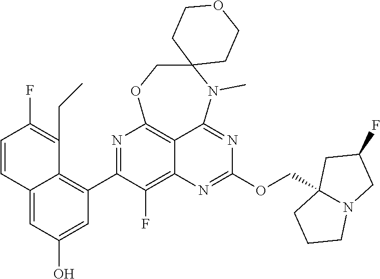 [SMILES: CCc1c(F)ccc2cc(O)cc(-c3nc4c5c(nc(OC[C@@]67CCCN6C[C@H](F)C7)nc5c3F)N(C)C3(CCOCC3)CO4)c12]

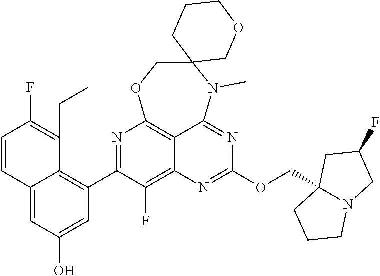 [SMILES: CCc1c(F)ccc2cc(O)cc(-c3nc4c5c(nc(OC[C@@]67CCCN6C[C@H](F)C7)nc5c3F)N(C)C3(CCCOC3)CO4)c12]

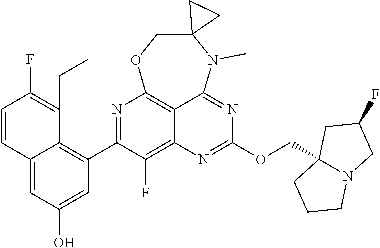 [SMILES: CCc1c(F)ccc2cc(O)cc(-c3nc4c5c(nc(OC[C@@]67CCCN6C[C@H](F)C7)nc5c3F)N(C)C3(CC3)CO4)c12]

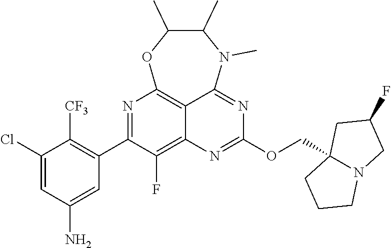 [SMILES: CC1Oc2nc(-c3cc(N)cc(Cl)c3C(F)(F)F)c(F)c3nc(OC[C@@]45CCCN4C[C@H](F)C5)nc(c23)N(C)C1C]

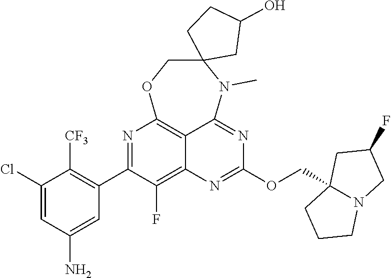 [SMILES: CN1c2nc(OC[C@@]34CCCN3C[C@H](F)C4)nc3c(F)c(-c4cc(N)cc(Cl)c4C(F)(F)F)nc(c23)OCC12CCC(O)C2]

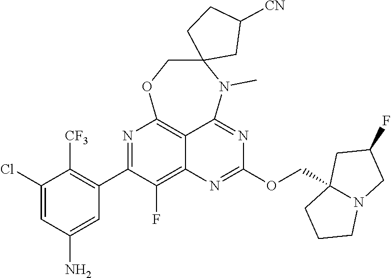 [SMILES: CN1c2nc(OC[C@@]34CCCN3C[C@H](F)C4)nc3c(F)c(-c4cc(N)cc(Cl)c4C(F)(F)F)nc(c23)OCC12CCC(C#N)C2]

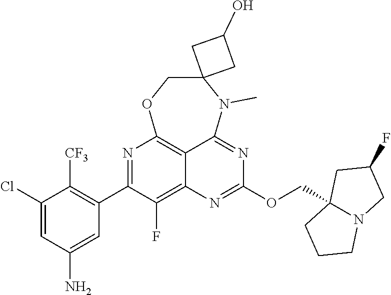 [SMILES: CN1c2nc(OC[C@@]34CCCN3C[C@H](F)C4)nc3c(F)c(-c4cc(N)cc(Cl)c4C(F)(F)F)nc(c23)OCC12CC(O)C2]

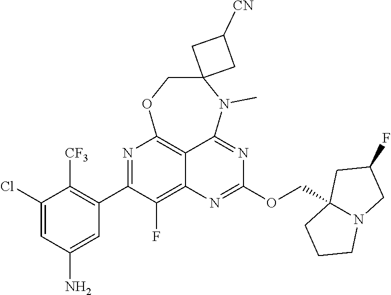 [SMILES: CN1c2nc(OC[C@@]34CCCN3C[C@H](F)C4)nc3c(F)c(-c4cc(N)cc(Cl)c4C(F)(F)F)nc(c23)OCC12CC(C#N)C2]

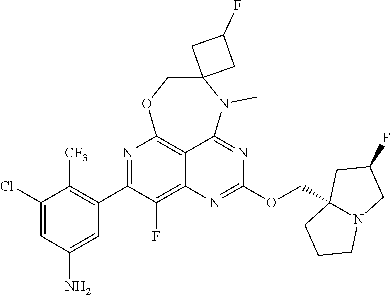 [SMILES: CN1c2nc(OC[C@@]34CCCN3C[C@H](F)C4)nc3c(F)c(-c4cc(N)cc(Cl)c4C(F)(F)F)nc(c23)OCC12CC(F)C2]

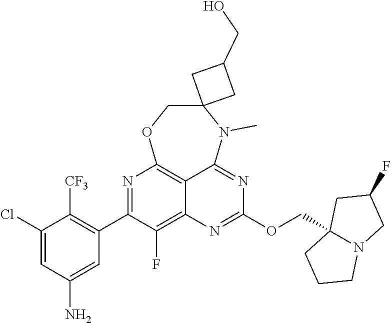 [SMILES: CN1c2nc(OC[C@@]34CCCN3C[C@H](F)C4)nc3c(F)c(-c4cc(N)cc(Cl)c4C(F)(F)F)nc(c23)OCC12CC(CO)C2]

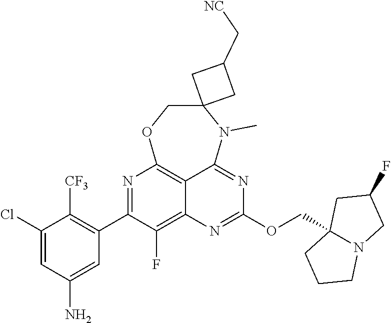 [SMILES: CN1c2nc(OC[C@@]34CCCN3C[C@H](F)C4)nc3c(F)c(-c4cc(N)cc(Cl)c4C(F)(F)F)nc(c23)OCC12CC(CC#N)C2]

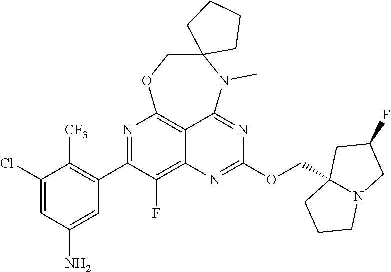 [SMILES: CN1c2nc(OC[C@@]34CCCN3C[C@H](F)C4)nc3c(F)c(-c4cc(N)cc(Cl)c4C(F)(F)F)nc(c23)OCC12CCCC2]

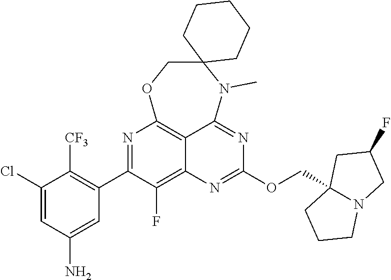 [SMILES: CN1c2nc(OC[C@@]34CCCN3C[C@H](F)C4)nc3c(F)c(-c4cc(N)cc(Cl)c4C(F)(F)F)nc(c23)OCC12CCCCC2]

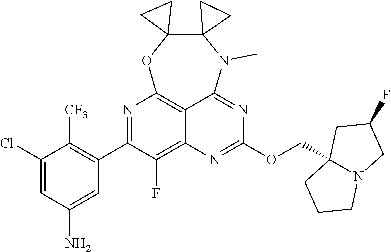 [SMILES: CN1c2nc(OC[C@@]34CCCN3C[C@H](F)C4)nc3c(F)c(-c4cc(N)cc(Cl)c4C(F)(F)F)nc(c23)OC2(CC2)C12CC2]

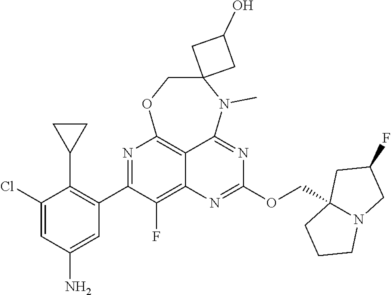 [SMILES: CN1c2nc(OC[C@@]34CCCN3C[C@H](F)C4)nc3c(F)c(-c4cc(N)cc(Cl)c4C4CC4)nc(c23)OCC12CC(O)C2]

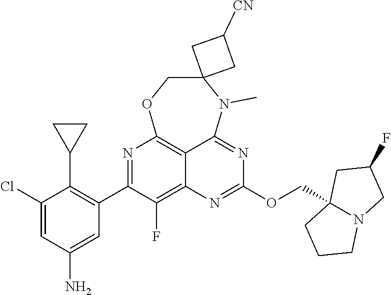 [SMILES: CN1c2nc(OC[C@@]34CCCN3C[C@H](F)C4)nc3c(F)c(-c4cc(N)cc(Cl)c4C4CC4)nc(c23)OCC12CC(C#N)C2]

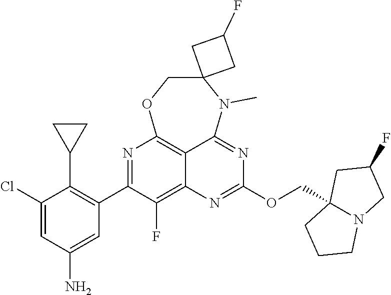 [SMILES: CN1c2nc(OC[C@@]34CCCN3C[C@H](F)C4)nc3c(F)c(-c4cc(N)cc(Cl)c4C4CC4)nc(c23)OCC12CC(F)C2]

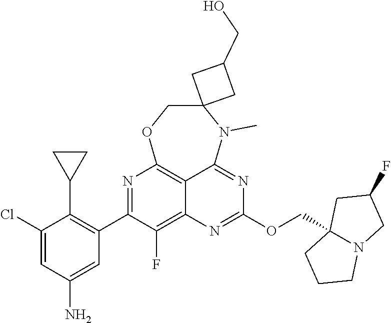 [SMILES: CN1c2nc(OC[C@@]34CCCN3C[C@H](F)C4)nc3c(F)c(-c4cc(N)cc(Cl)c4C4CC4)nc(c23)OCC12CC(CO)C2]

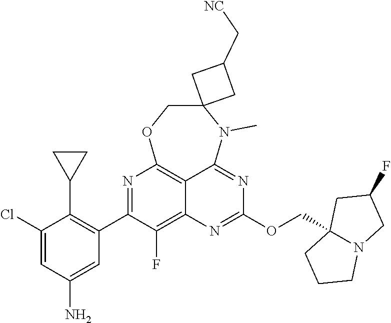 [SMILES: CN1c2nc(OC[C@@]34CCCN3C[C@H](F)C4)nc3c(F)c(-c4cc(N)cc(Cl)c4C4CC4)nc(c23)OCC12CC(CC#N)C2]

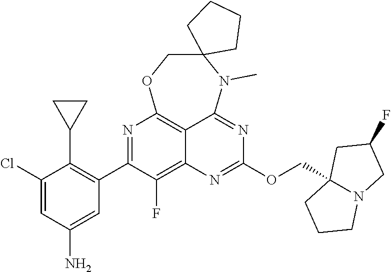 [SMILES: CN1c2nc(OC[C@@]34CCCN3C[C@H](F)C4)nc3c(F)c(-c4cc(N)cc(Cl)c4C4CC4)nc(c23)OCC12CCCC2]

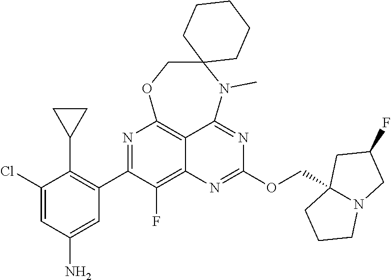 [SMILES: CN1c2nc(OC[C@@]34CCCN3C[C@H](F)C4)nc3c(F)c(-c4cc(N)cc(Cl)c4C4CC4)nc(c23)OCC12CCCCC2]

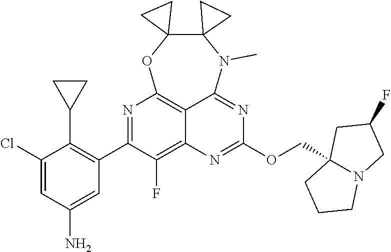 [SMILES: CN1c2nc(OC[C@@]34CCCN3C[C@H](F)C4)nc3c(F)c(-c4cc(N)cc(Cl)c4C4CC4)nc(c23)OC2(CC2)C12CC2]

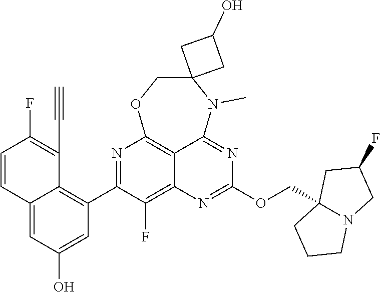 [SMILES: C#Cc1c(F)ccc2cc(O)cc(-c3nc4c5c(nc(OC[C@@]67CCCN6C[C@H](F)C7)nc5c3F)N(C)C3(CO4)CC(O)C3)c12]

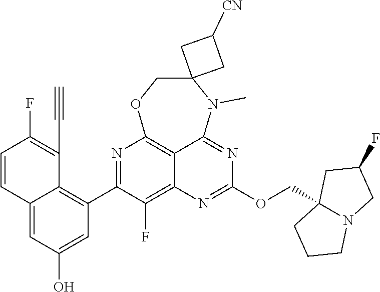 [SMILES: C#Cc1c(F)ccc2cc(O)cc(-c3nc4c5c(nc(OC[C@@]67CCCN6C[C@H](F)C7)nc5c3F)N(C)C3(CO4)CC(C#N)C3)c12]

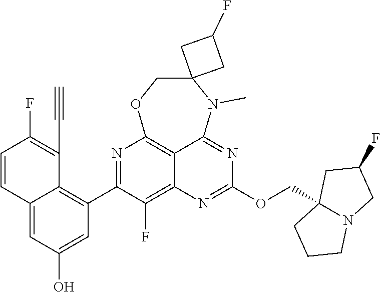 [SMILES: C#Cc1c(F)ccc2cc(O)cc(-c3nc4c5c(nc(OC[C@@]67CCCN6C[C@H](F)C7)nc5c3F)N(C)C3(CO4)CC(F)C3)c12]

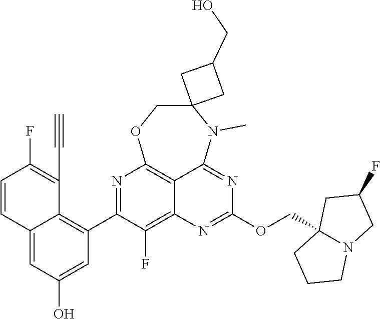 [SMILES: C#Cc1c(F)ccc2cc(O)cc(-c3nc4c5c(nc(OC[C@@]67CCCN6C[C@H](F)C7)nc5c3F)N(C)C3(CO4)CC(CO)C3)c12]

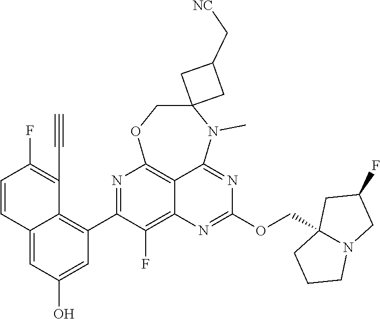 [SMILES: C#Cc1c(F)ccc2cc(O)cc(-c3nc4c5c(nc(OC[C@@]67CCCN6C[C@H](F)C7)nc5c3F)N(C)C3(CO4)CC(CC#N)C3)c12]

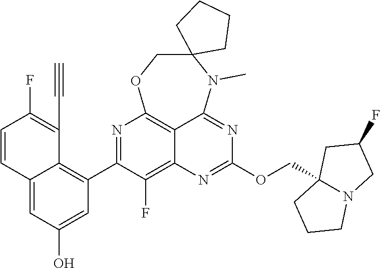 [SMILES: C#Cc1c(F)ccc2cc(O)cc(-c3nc4c5c(nc(OC[C@@]67CCCN6C[C@H](F)C7)nc5c3F)N(C)C3(CCCC3)CO4)c12]

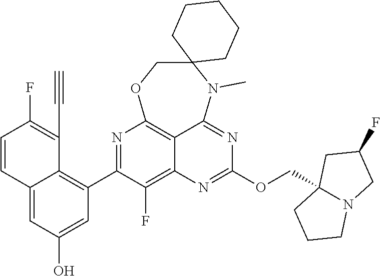 [SMILES: C#Cc1c(F)ccc2cc(O)cc(-c3nc4c5c(nc(OC[C@@]67CCCN6C[C@H](F)C7)nc5c3F)N(C)C3(CCCCC3)CO4)c12]

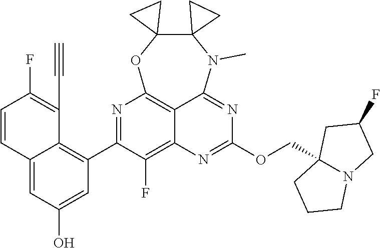 [SMILES: C#Cc1c(F)ccc2cc(O)cc(-c3nc4c5c(nc(OC[C@@]67CCCN6C[C@H](F)C7)nc5c3F)N(C)C3(CC3)C3(CC3)O4)c12]

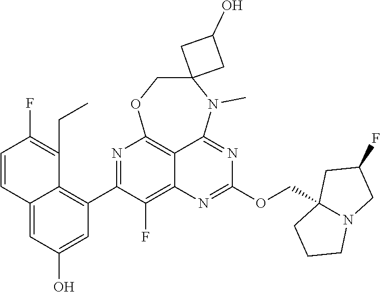 [SMILES: CCc1c(F)ccc2cc(O)cc(-c3nc4c5c(nc(OC[C@@]67CCCN6C[C@H](F)C7)nc5c3F)N(C)C3(CO4)CC(O)C3)c12]

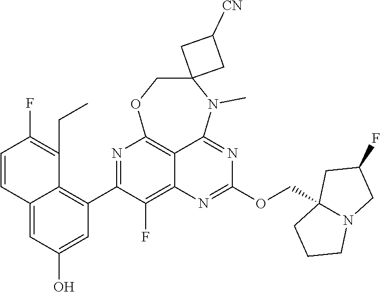 [SMILES: CCc1c(F)ccc2cc(O)cc(-c3nc4c5c(nc(OC[C@@]67CCCN6C[C@H](F)C7)nc5c3F)N(C)C3(CO4)CC(C#N)C3)c12]

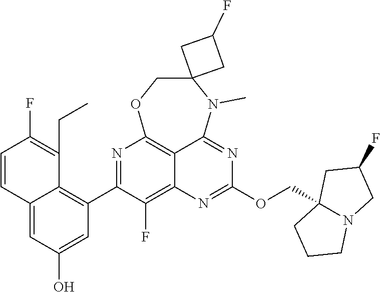 [SMILES: CCc1c(F)ccc2cc(O)cc(-c3nc4c5c(nc(OC[C@@]67CCCN6C[C@H](F)C7)nc5c3F)N(C)C3(CO4)CC(F)C3)c12]

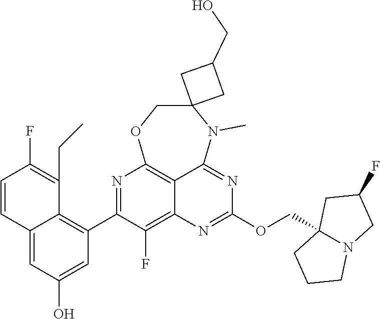 [SMILES: CCc1c(F)ccc2cc(O)cc(-c3nc4c5c(nc(OC[C@@]67CCCN6C[C@H](F)C7)nc5c3F)N(C)C3(CO4)CC(CO)C3)c12]

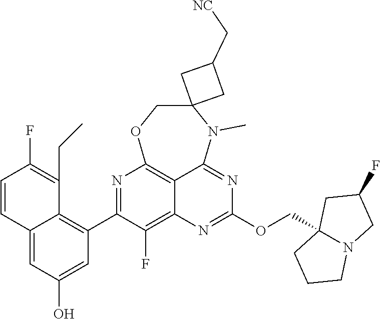 [SMILES: CCc1c(F)ccc2cc(O)cc(-c3nc4c5c(nc(OC[C@@]67CCCN6C[C@H](F)C7)nc5c3F)N(C)C3(CO4)CC(CC#N)C3)c12]

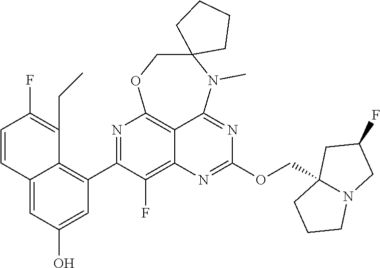 [SMILES: CCc1c(F)ccc2cc(O)cc(-c3nc4c5c(nc(OC[C@@]67CCCN6C[C@H](F)C7)nc5c3F)N(C)C3(CCCC3)CO4)c12]

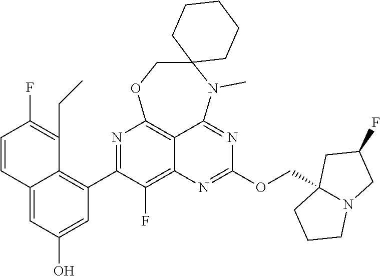 [SMILES: CCc1c(F)ccc2cc(O)cc(-c3nc4c5c(nc(OC[C@@]67CCCN6C[C@H](F)C7)nc5c3F)N(C)C3(CCCCC3)CO4)c12]

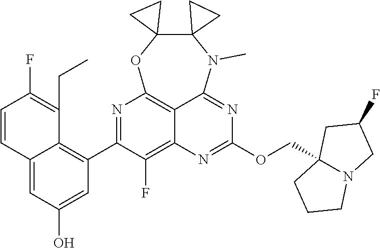 [SMILES: CCc1c(F)ccc2cc(O)cc(-c3nc4c5c(nc(OC[C@@]67CCCN6C[C@H](F)C7)nc5c3F)N(C)C3(CC3)C3(CC3)O4)c12]

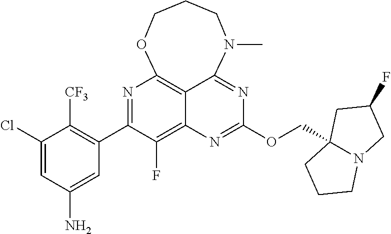 [SMILES: CN1CCCOc2nc(-c3cc(N)cc(Cl)c3C(F)(F)F)c(F)c3nc(OC[C@@]45CCCN4C[C@H](F)C5)nc1c23]

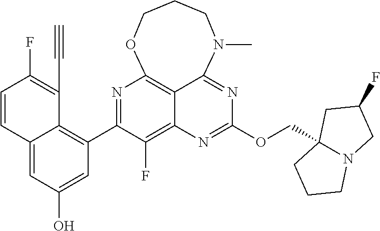 [SMILES: C#Cc1c(F)ccc2cc(O)cc(-c3nc4c5c(nc(OC[C@@]67CCCN6C[C@H](F)C7)nc5c3F)N(C)CCCO4)c12]

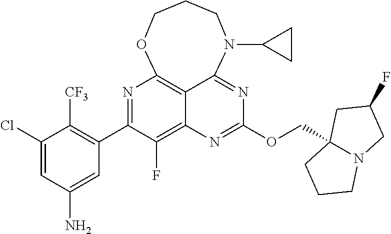 [SMILES: Nc1cc(Cl)c(C(F)(F)F)c(-c2nc3c4c(nc(OC[C@@]56CCCN5C[C@H](F)C6)nc4c2F)N(C2CC2)CCCO3)c1]

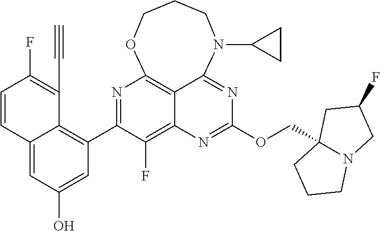 [SMILES: C#Cc1c(F)ccc2cc(O)cc(-c3nc4c5c(nc(OC[C@@]67CCCN6C[C@H](F)C7)nc5c3F)N(C3CC3)CCCO4)c12]

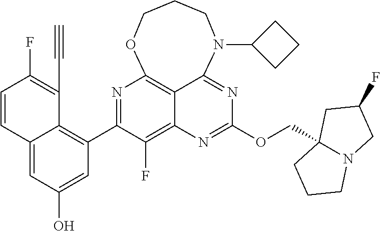 [SMILES: C#Cc1c(F)ccc2cc(O)cc(-c3nc4c5c(nc(OC[C@@]67CCCN6C[C@H](F)C7)nc5c3F)N(C3CCC3)CCCO4)c12]

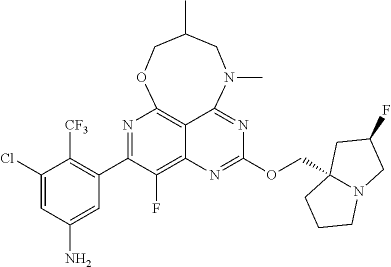 [SMILES: CC1COc2nc(-c3cc(N)cc(Cl)c3C(F)(F)F)c(F)c3nc(OC[C@@]45CCCN4C[C@H](F)C5)nc(c23)N(C)C1]

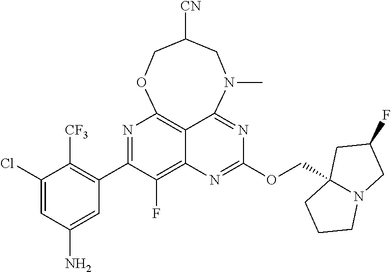 [SMILES: CN1CC(C#N)COc2nc(-c3cc(N)cc(Cl)c3C(F)(F)F)c(F)c3nc(OC[C@@]45CCCN4C[C@H](F)C5)nc1c23]

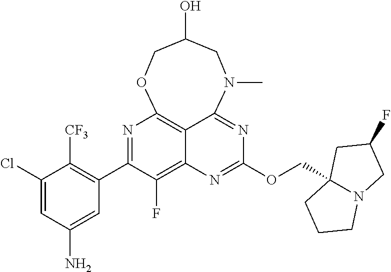 [SMILES: CN1CC(O)COc2nc(-c3cc(N)cc(Cl)c3C(F)(F)F)c(F)c3nc(OC[C@@]45CCCN4C[C@H](F)C5)nc1c23]

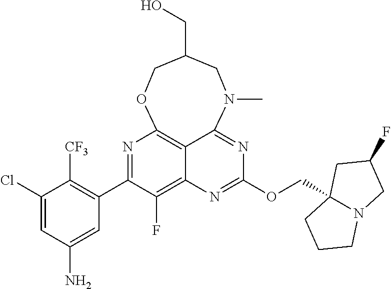 [SMILES: CN1CC(CO)COc2nc(-c3cc(N)cc(Cl)c3C(F)(F)F)c(F)c3nc(OC[C@@]45CCCN4C[C@H](F)C5)nc1c23]

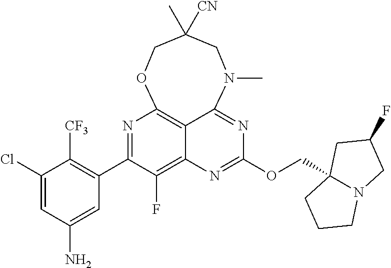 [SMILES: CN1CC(C)(C#N)COc2nc(-c3cc(N)cc(Cl)c3C(F)(F)F)c(F)c3nc(OC[C@@]45CCCN4C[C@H](F)C5)nc1c23]

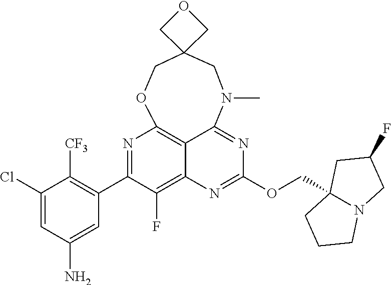 [SMILES: CN1CC2(COC2)COc2nc(-c3cc(N)cc(Cl)c3C(F)(F)F)c(F)c3nc(OC[C@@]45CCCN4C[C@H](F)C5)nc1c23]

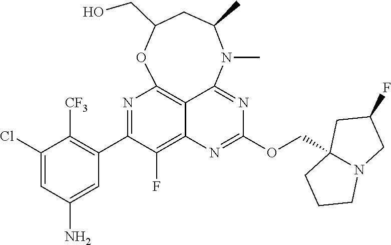 [SMILES: C[C@@H]1CC(CO)Oc2nc(-c3cc(N)cc(Cl)c3C(F)(F)F)c(F)c3nc(OC[C@@]45CCCN4C[C@H](F)C5)nc(c23)N1C]

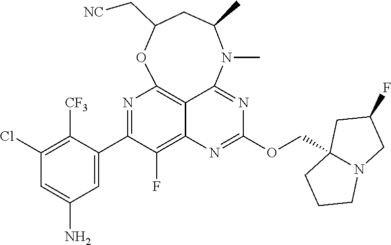 [SMILES: C[C@@H]1CC(CC#N)Oc2nc(-c3cc(N)cc(Cl)c3C(F)(F)F)c(F)c3nc(OC[C@@]45CCCN4C[C@H](F)C5)nc(c23)N1C]

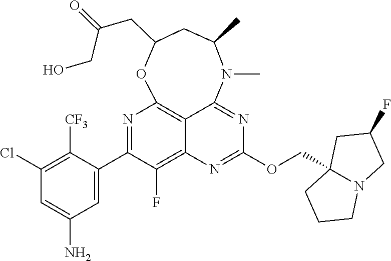 [SMILES: C[C@@H]1CC(CC(=O)CO)Oc2nc(-c3cc(N)cc(Cl)c3C(F)(F)F)c(F)c3nc(OC[C@@]45CCCN4C[C@H](F)C5)nc(c23)N1C]

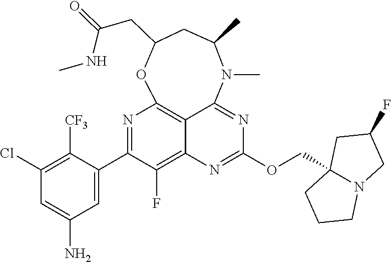 [SMILES: CNC(=O)CC1C[C@@H](C)N(C)c2nc(OC[C@@]34CCCN3C[C@H](F)C4)nc3c(F)c(-c4cc(N)cc(Cl)c4C(F)(F)F)nc(c23)O1]

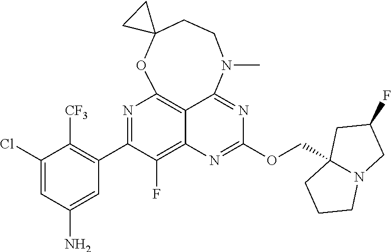 [SMILES: CN1CCC2(CC2)Oc2nc(-c3cc(N)cc(Cl)c3C(F)(F)F)c(F)c3nc(OC[C@@]45CCCN4C[C@H](F)C5)nc1c23]

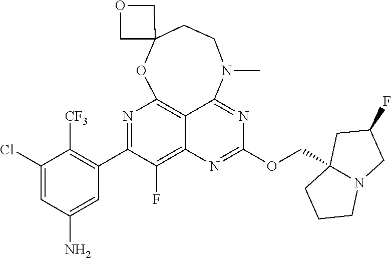 [SMILES: CN1CCC2(COC2)Oc2nc(-c3cc(N)cc(Cl)c3C(F)(F)F)c(F)c3nc(OC[C@@]45CCCN4C[C@H](F)C5)nc1c23]

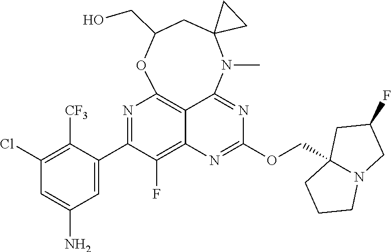 [SMILES: CN1c2nc(OC[C@@]34CCCN3C[C@H](F)C4)nc3c(F)c(-c4cc(N)cc(Cl)c4C(F)(F)F)nc(c23)OC(CO)CC12CC2]

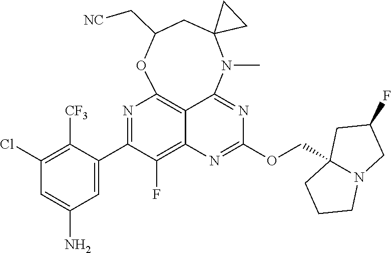 [SMILES: CN1c2nc(OC[C@@]34CCCN3C[C@H](F)C4)nc3c(F)c(-c4cc(N)cc(Cl)c4C(F)(F)F)nc(c23)OC(CC#N)CC12CC2]

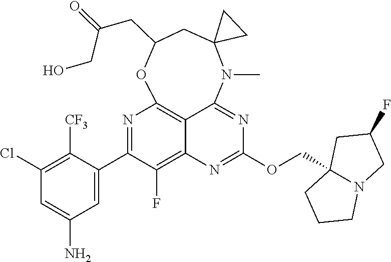 [SMILES: CN1c2nc(OC[C@@]34CCCN3C[C@H](F)C4)nc3c(F)c(-c4cc(N)cc(Cl)c4C(F)(F)F)nc(c23)OC(CC(=O)CO)CC12CC2]

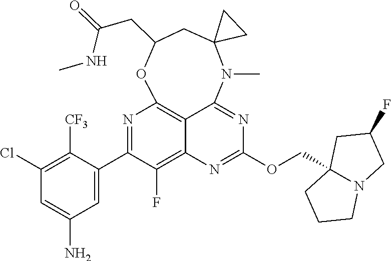 [SMILES: CNC(=O)CC1CC2(CC2)N(C)c2nc(OC[C@@]34CCCN3C[C@H](F)C4)nc3c(F)c(-c4cc(N)cc(Cl)c4C(F)(F)F)nc(c23)O1]

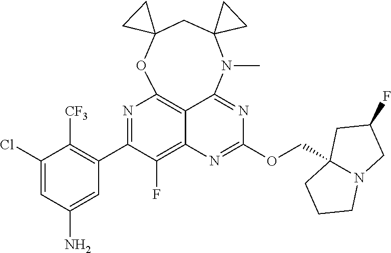 [SMILES: CN1c2nc(OC[C@@]34CCCN3C[C@H](F)C4)nc3c(F)c(-c4cc(N)cc(Cl)c4C(F)(F)F)nc(c23)OC2(CC2)CC12CC2]

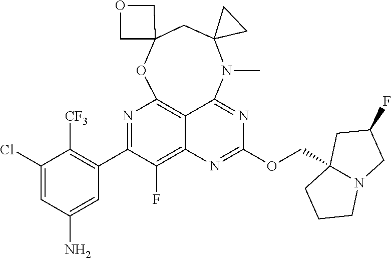 [SMILES: CN1c2nc(OC[C@@]34CCCN3C[C@H](F)C4)nc3c(F)c(-c4cc(N)cc(Cl)c4C(F)(F)F)nc(c23)OC2(COC2)CC12CC2]

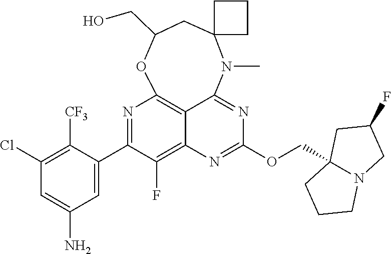 [SMILES: CN1c2nc(OC[C@@]34CCCN3C[C@H](F)C4)nc3c(F)c(-c4cc(N)cc(Cl)c4C(F)(F)F)nc(c23)OC(CO)CC12CCC2]

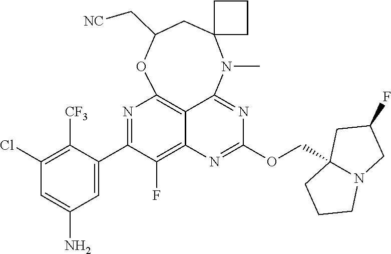 [SMILES: CN1c2nc(OC[C@@]34CCCN3C[C@H](F)C4)nc3c(F)c(-c4cc(N)cc(Cl)c4C(F)(F)F)nc(c23)OC(CC#N)CC12CCC2]

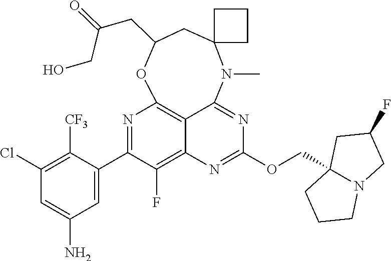 [SMILES: CN1c2nc(OC[C@@]34CCCN3C[C@H](F)C4)nc3c(F)c(-c4cc(N)cc(Cl)c4C(F)(F)F)nc(c23)OC(CC(=O)CO)CC12CCC2]

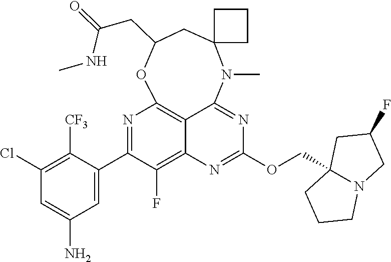 [SMILES: CNC(=O)CC1CC2(CCC2)N(C)c2nc(OC[C@@]34CCCN3C[C@H](F)C4)nc3c(F)c(-c4cc(N)cc(Cl)c4C(F)(F)F)nc(c23)O1]

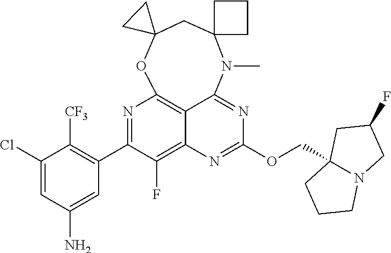 [SMILES: CN1c2nc(OC[C@@]34CCCN3C[C@H](F)C4)nc3c(F)c(-c4cc(N)cc(Cl)c4C(F)(F)F)nc(c23)OC2(CC2)CC12CCC2]

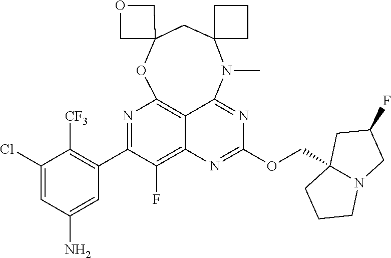 [SMILES: CN1c2nc(OC[C@@]34CCCN3C[C@H](F)C4)nc3c(F)c(-c4cc(N)cc(Cl)c4C(F)(F)F)nc(c23)OC2(COC2)CC12CCC2]

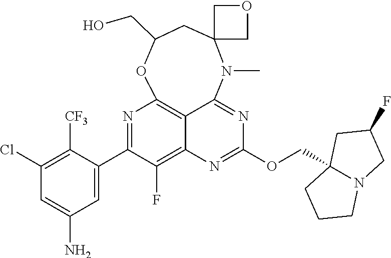 [SMILES: CN1c2nc(OC[C@@]34CCCN3C[C@H](F)C4)nc3c(F)c(-c4cc(N)cc(Cl)c4C(F)(F)F)nc(c23)OC(CO)CC12COC2]

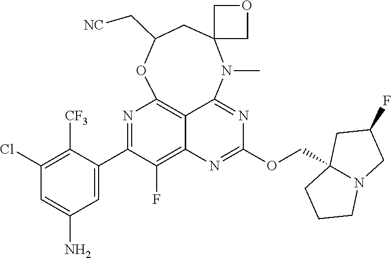 [SMILES: CN1c2nc(OC[C@@]34CCCN3C[C@H](F)C4)nc3c(F)c(-c4cc(N)cc(Cl)c4C(F)(F)F)nc(c23)OC(CC#N)CC12COC2]

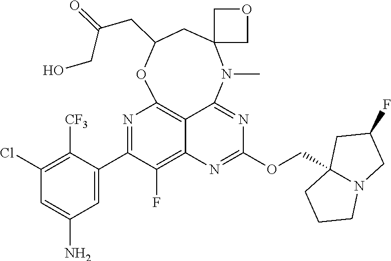 [SMILES: CN1c2nc(OC[C@@]34CCCN3C[C@H](F)C4)nc3c(F)c(-c4cc(N)cc(Cl)c4C(F)(F)F)nc(c23)OC(CC(=O)CO)CC12COC2]

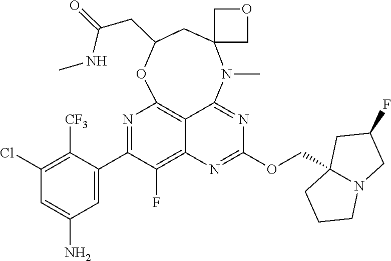 [SMILES: CNC(=O)CC1CC2(COC2)N(C)c2nc(OC[C@@]34CCCN3C[C@H](F)C4)nc3c(F)c(-c4cc(N)cc(Cl)c4C(F)(F)F)nc(c23)O1]

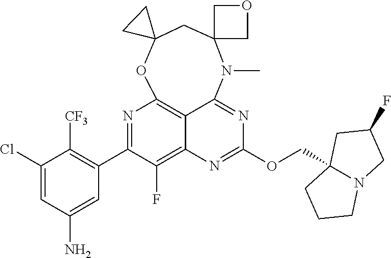 [SMILES: CN1c2nc(OC[C@@]34CCCN3C[C@H](F)C4)nc3c(F)c(-c4cc(N)cc(Cl)c4C(F)(F)F)nc(c23)OC2(CC2)CC12COC2]

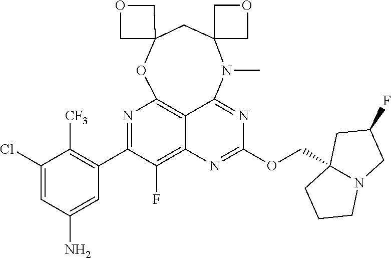 [SMILES: CN1c2nc(OC[C@@]34CCCN3C[C@H](F)C4)nc3c(F)c(-c4cc(N)cc(Cl)c4C(F)(F)F)nc(c23)OC2(COC2)CC12COC2]

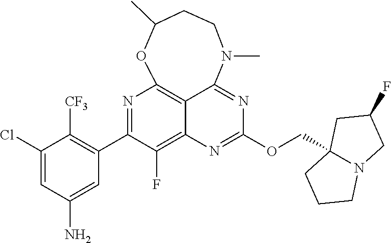 [SMILES: CC1CCN(C)c2nc(OC[C@@]34CCCN3C[C@H](F)C4)nc3c(F)c(-c4cc(N)cc(Cl)c4C(F)(F)F)nc(c23)O1]

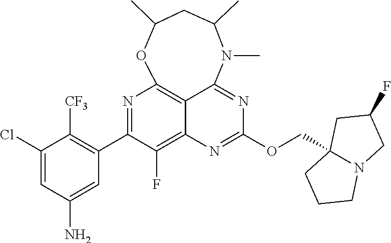 [SMILES: CC1CC(C)N(C)c2nc(OC[C@@]34CCCN3C[C@H](F)C4)nc3c(F)c(-c4cc(N)cc(Cl)c4C(F)(F)F)nc(c23)O1]

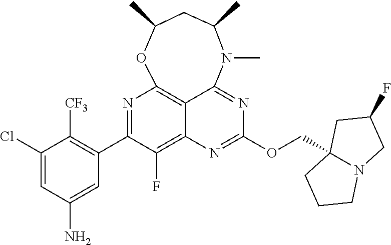 [SMILES: C[C@@H]1C[C@H](C)Oc2nc(-c3cc(N)cc(Cl)c3C(F)(F)F)c(F)c3nc(OC[C@@]45CCCN4C[C@H](F)C5)nc(c23)N1C]

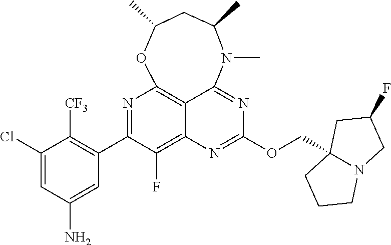 [SMILES: C[C@@H]1C[C@@H](C)N(C)c2nc(OC[C@@]34CCCN3C[C@H](F)C4)nc3c(F)c(-c4cc(N)cc(Cl)c4C(F)(F)F)nc(c23)O1]

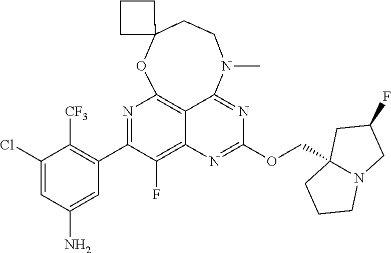 [SMILES: CN1CCC2(CCC2)Oc2nc(-c3cc(N)cc(Cl)c3C(F)(F)F)c(F)c3nc(OC[C@@]45CCCN4C[C@H](F)C5)nc1c23]

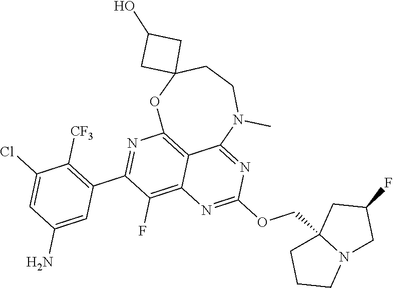 [SMILES: CN1CCC2(CC(O)C2)Oc2nc(-c3cc(N)cc(Cl)c3C(F)(F)F)c(F)c3nc(OC[C@@]45CCCN4C[C@H](F)C5)nc1c23]

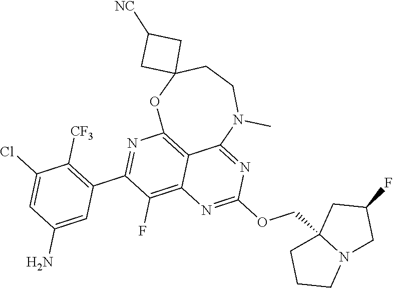 [SMILES: CN1CCC2(CC(C#N)C2)Oc2nc(-c3cc(N)cc(Cl)c3C(F)(F)F)c(F)c3nc(OC[C@@]45CCCN4C[C@H](F)C5)nc1c23]

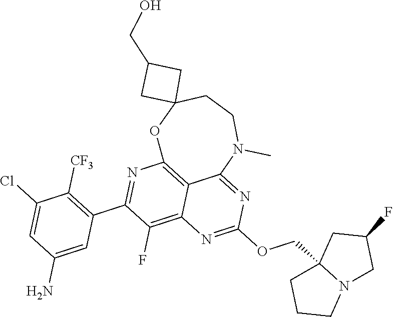 [SMILES: CN1CCC2(CC(CO)C2)Oc2nc(-c3cc(N)cc(Cl)c3C(F)(F)F)c(F)c3nc(OC[C@@]45CCCN4C[C@H](F)C5)nc1c23]

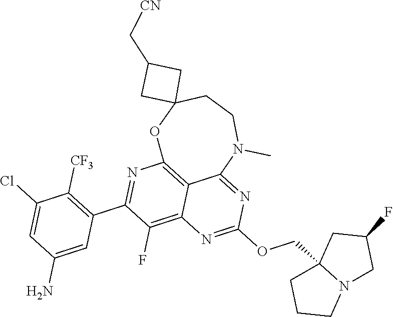 [SMILES: CN1CCC2(CC(CC#N)C2)Oc2nc(-c3cc(N)cc(Cl)c3C(F)(F)F)c(F)c3nc(OC[C@@]45CCCN4C[C@H](F)C5)nc1c23]

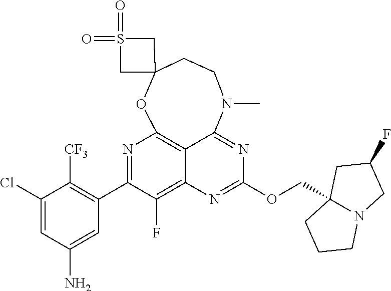 [SMILES: CN1CCC2(CS(=O)(=O)C2)Oc2nc(-c3cc(N)cc(Cl)c3C(F)(F)F)c(F)c3nc(OC[C@@]45CCCN4C[C@H](F)C5)nc1c23]

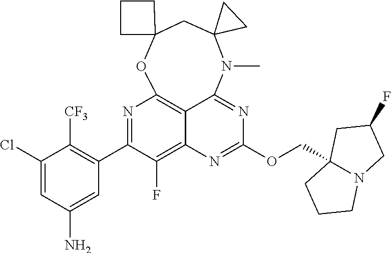 [SMILES: CN1c2nc(OC[C@@]34CCCN3C[C@H](F)C4)nc3c(F)c(-c4cc(N)cc(Cl)c4C(F)(F)F)nc(c23)OC2(CCC2)CC12CC2]

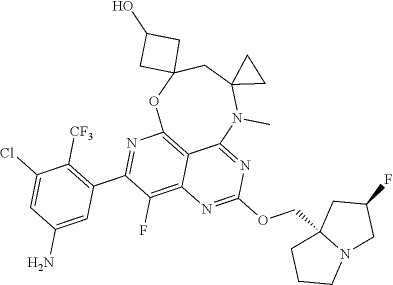 [SMILES: CN1c2nc(OC[C@@]34CCCN3C[C@H](F)C4)nc3c(F)c(-c4cc(N)cc(Cl)c4C(F)(F)F)nc(c23)OC2(CC(O)C2)CC12CC2]

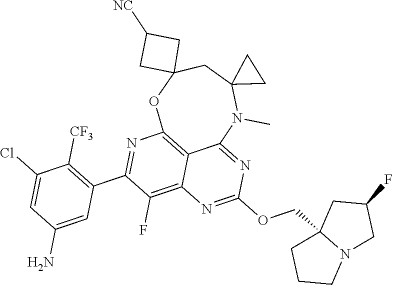 [SMILES: CN1c2nc(OC[C@@]34CCCN3C[C@H](F)C4)nc3c(F)c(-c4cc(N)cc(Cl)c4C(F)(F)F)nc(c23)OC2(CC(C#N)C2)CC12CC2]

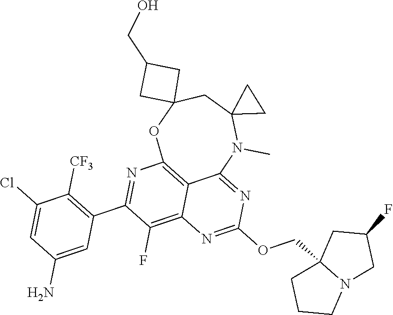 [SMILES: CN1c2nc(OC[C@@]34CCCN3C[C@H](F)C4)nc3c(F)c(-c4cc(N)cc(Cl)c4C(F)(F)F)nc(c23)OC2(CC(CO)C2)CC12CC2]

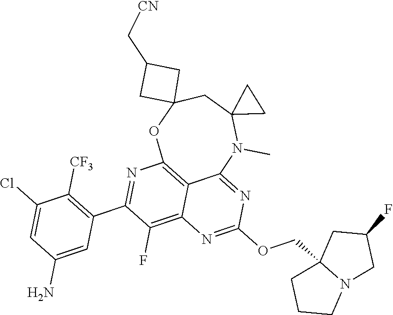 [SMILES: CN1c2nc(OC[C@@]34CCCN3C[C@H](F)C4)nc3c(F)c(-c4cc(N)cc(Cl)c4C(F)(F)F)nc(c23)OC2(CC(CC#N)C2)CC12CC2]

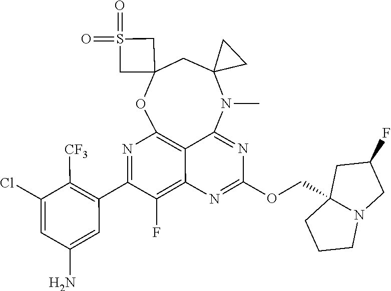 [SMILES: CN1c2nc(OC[C@@]34CCCN3C[C@H](F)C4)nc3c(F)c(-c4cc(N)cc(Cl)c4C(F)(F)F)nc(c23)OC2(CC13CC3)CS(=O)(=O)C2]

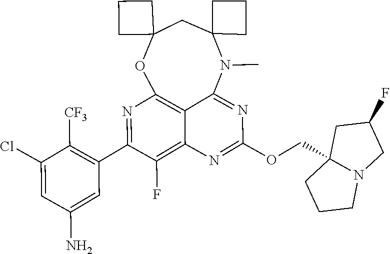 [SMILES: CN1c2nc(OC[C@@]34CCCN3C[C@H](F)C4)nc3c(F)c(-c4cc(N)cc(Cl)c4C(F)(F)F)nc(c23)OC2(CCC2)CC12CCC2]

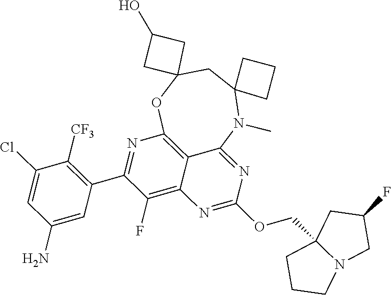 [SMILES: CN1c2nc(OC[C@@]34CCCN3C[C@H](F)C4)nc3c(F)c(-c4cc(N)cc(Cl)c4C(F)(F)F)nc(c23)OC2(CC(O)C2)CC12CCC2]

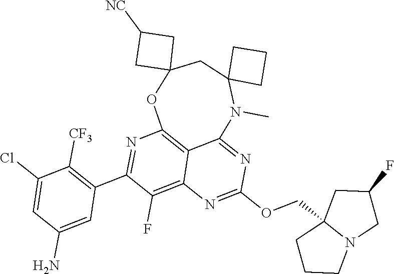 [SMILES: CN1c2nc(OC[C@@]34CCCN3C[C@H](F)C4)nc3c(F)c(-c4cc(N)cc(Cl)c4C(F)(F)F)nc(c23)OC2(CC(C#N)C2)CC12CCC2]

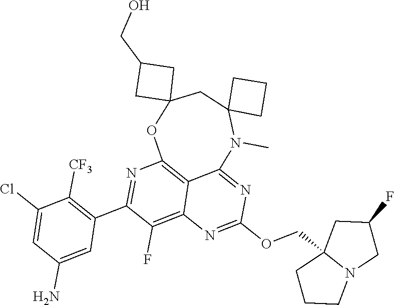 [SMILES: CN1c2nc(OC[C@@]34CCCN3C[C@H](F)C4)nc3c(F)c(-c4cc(N)cc(Cl)c4C(F)(F)F)nc(c23)OC2(CC(CO)C2)CC12CCC2]

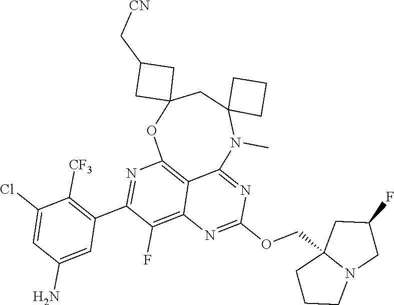 [SMILES: CN1c2nc(OC[C@@]34CCCN3C[C@H](F)C4)nc3c(F)c(-c4cc(N)cc(Cl)c4C(F)(F)F)nc(c23)OC2(CC(CC#N)C2)CC12CCC2]

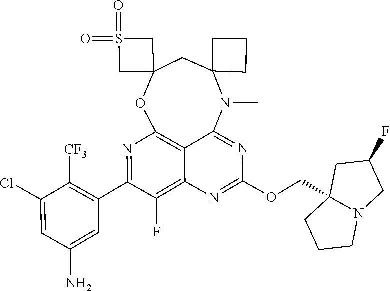 [SMILES: CN1c2nc(OC[C@@]34CCCN3C[C@H](F)C4)nc3c(F)c(-c4cc(N)cc(Cl)c4C(F)(F)F)nc(c23)OC2(CC13CCC3)CS(=O)(=O)C2]

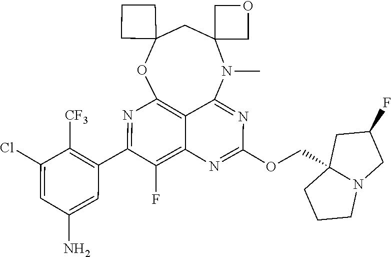 [SMILES: CN1c2nc(OC[C@@]34CCCN3C[C@H](F)C4)nc3c(F)c(-c4cc(N)cc(Cl)c4C(F)(F)F)nc(c23)OC2(CCC2)CC12COC2]

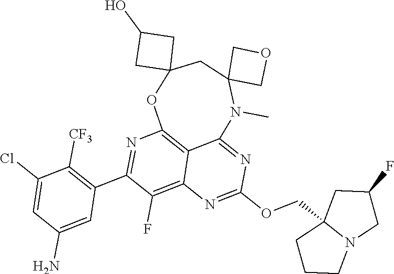 [SMILES: CN1c2nc(OC[C@@]34CCCN3C[C@H](F)C4)nc3c(F)c(-c4cc(N)cc(Cl)c4C(F)(F)F)nc(c23)OC2(CC(O)C2)CC12COC2]

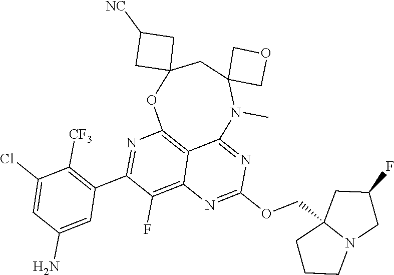 [SMILES: CN1c2nc(OC[C@@]34CCCN3C[C@H](F)C4)nc3c(F)c(-c4cc(N)cc(Cl)c4C(F)(F)F)nc(c23)OC2(CC(C#N)C2)CC12COC2]

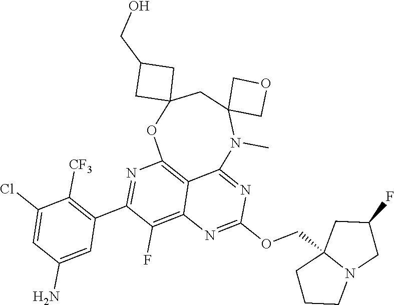 [SMILES: CN1c2nc(OC[C@@]34CCCN3C[C@H](F)C4)nc3c(F)c(-c4cc(N)cc(Cl)c4C(F)(F)F)nc(c23)OC2(CC(CO)C2)CC12COC2]

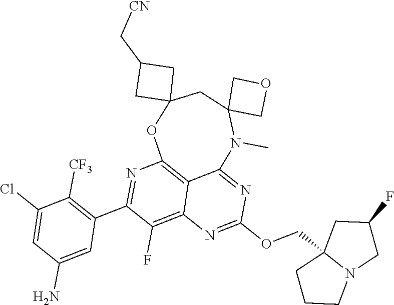 [SMILES: CN1c2nc(OC[C@@]34CCCN3C[C@H](F)C4)nc3c(F)c(-c4cc(N)cc(Cl)c4C(F)(F)F)nc(c23)OC2(CC(CC#N)C2)CC12COC2]

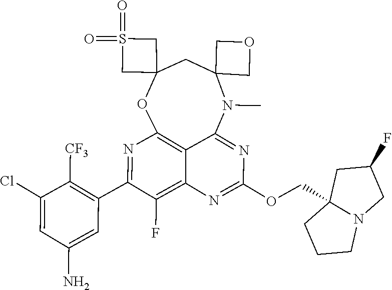 [SMILES: CN1c2nc(OC[C@@]34CCCN3C[C@H](F)C4)nc3c(F)c(-c4cc(N)cc(Cl)c4C(F)(F)F)nc(c23)OC2(CC13COC3)CS(=O)(=O)C2]

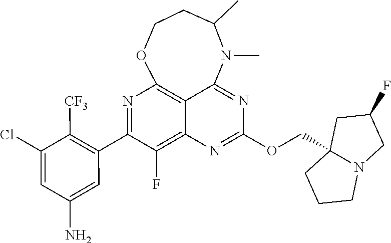 [SMILES: CC1CCOc2nc(-c3cc(N)cc(Cl)c3C(F)(F)F)c(F)c3nc(OC[C@@]45CCCN4C[C@H](F)C5)nc(c23)N1C]

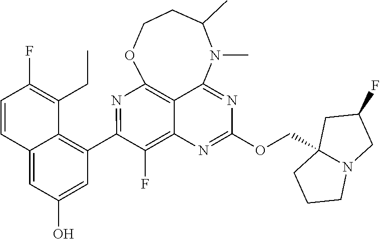 [SMILES: CCc1c(F)ccc2cc(O)cc(-c3nc4c5c(nc(OC[C@@]67CCCN6C[C@H](F)C7)nc5c3F)N(C)C(C)CCO4)c12]

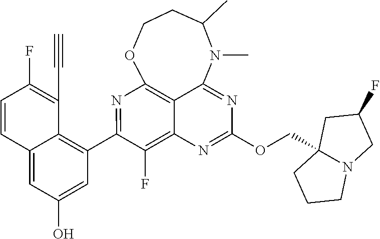 [SMILES: C#Cc1c(F)ccc2cc(O)cc(-c3nc4c5c(nc(OC[C@@]67CCCN6C[C@H](F)C7)nc5c3F)N(C)C(C)CCO4)c12]

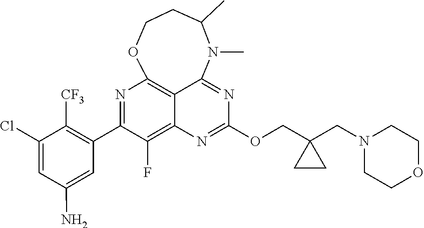 [SMILES: CC1CCOc2nc(-c3cc(N)cc(Cl)c3C(F)(F)F)c(F)c3nc(OCC4(CN5CCOCC5)CC4)nc(c23)N1C]

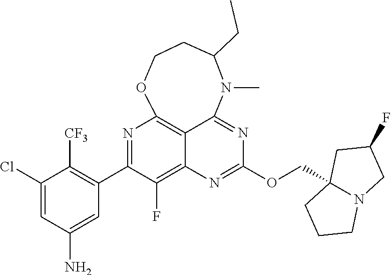 [SMILES: CCC1CCOc2nc(-c3cc(N)cc(Cl)c3C(F)(F)F)c(F)c3nc(OC[C@@]45CCCN4C[C@H](F)C5)nc(c23)N1C]

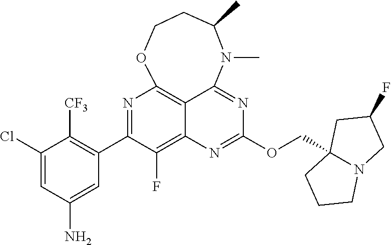 [SMILES: C[C@@H]1CCOc2nc(-c3cc(N)cc(Cl)c3C(F)(F)F)c(F)c3nc(OC[C@@]45CCCN4C[C@H](F)C5)nc(c23)N1C]

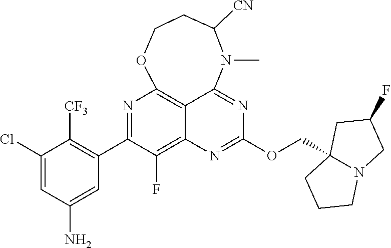 [SMILES: CN1c2nc(OC[C@@]34CCCN3C[C@H](F)C4)nc3c(F)c(-c4cc(N)cc(Cl)c4C(F)(F)F)nc(c23)OCCC1C#N]

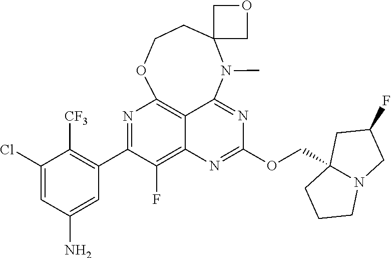 [SMILES: CN1c2nc(OC[C@@]34CCCN3C[C@H](F)C4)nc3c(F)c(-c4cc(N)cc(Cl)c4C(F)(F)F)nc(c23)OCCC12COC2]

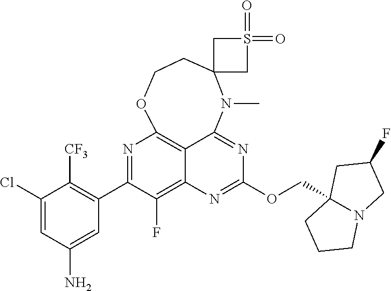 [SMILES: CN1c2nc(OC[C@@]34CCCN3C[C@H](F)C4)nc3c(F)c(-c4cc(N)cc(Cl)c4C(F)(F)F)nc(c23)OCCC12CS(=O)(=O)C2]

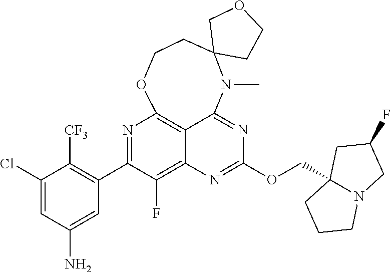 [SMILES: CN1c2nc(OC[C@@]34CCCN3C[C@H](F)C4)nc3c(F)c(-c4cc(N)cc(Cl)c4C(F)(F)F)nc(c23)OCCC12CCOC2]

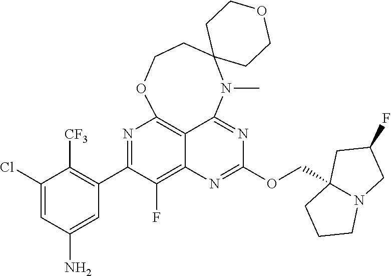 [SMILES: CN1c2nc(OC[C@@]34CCCN3C[C@H](F)C4)nc3c(F)c(-c4cc(N)cc(Cl)c4C(F)(F)F)nc(c23)OCCC12CCOCC2]

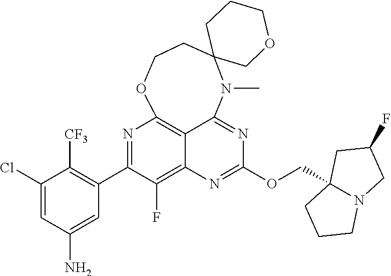 [SMILES: CN1c2nc(OC[C@@]34CCCN3C[C@H](F)C4)nc3c(F)c(-c4cc(N)cc(Cl)c4C(F)(F)F)nc(c23)OCCC12CCCOC2]

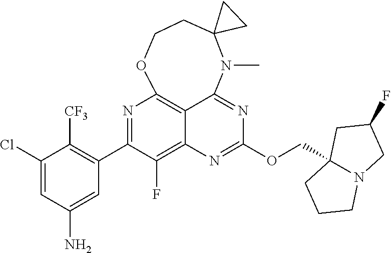 [SMILES: CN1c2nc(OC[C@@]34CCCN3C[C@H](F)C4)nc3c(F)c(-c4cc(N)cc(Cl)c4C(F)(F)F)nc(c23)OCCC12CC2]

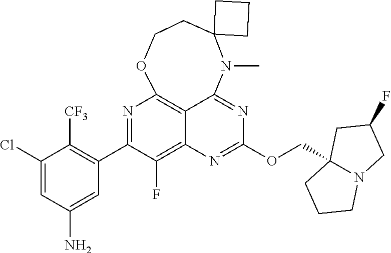 [SMILES: CN1c2nc(OC[C@@]34CCCN3C[C@H](F)C4)nc3c(F)c(-c4cc(N)cc(Cl)c4C(F)(F)F)nc(c23)OCCC12CCC2]

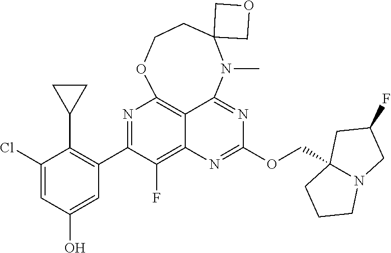 [SMILES: CN1c2nc(OC[C@@]34CCCN3C[C@H](F)C4)nc3c(F)c(-c4cc(O)cc(Cl)c4C4CC4)nc(c23)OCCC12COC2]

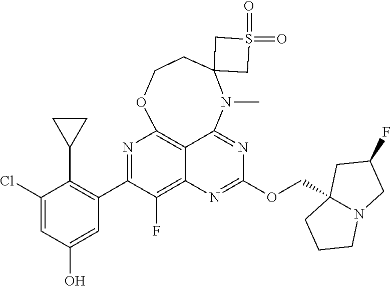 [SMILES: CN1c2nc(OC[C@@]34CCCN3C[C@H](F)C4)nc3c(F)c(-c4cc(O)cc(Cl)c4C4CC4)nc(c23)OCCC12CS(=O)(=O)C2]

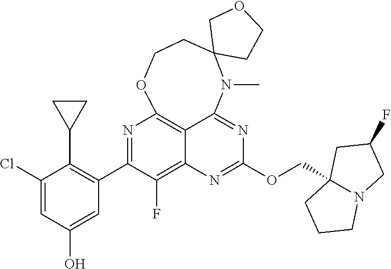 [SMILES: CN1c2nc(OC[C@@]34CCCN3C[C@H](F)C4)nc3c(F)c(-c4cc(O)cc(Cl)c4C4CC4)nc(c23)OCCC12CCOC2]

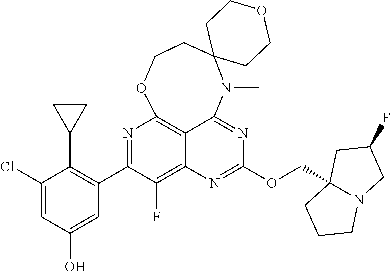 [SMILES: CN1c2nc(OC[C@@]34CCCN3C[C@H](F)C4)nc3c(F)c(-c4cc(O)cc(Cl)c4C4CC4)nc(c23)OCCC12CCOCC2]

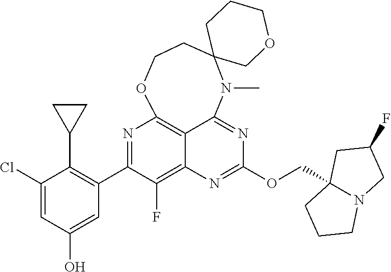 [SMILES: CN1c2nc(OC[C@@]34CCCN3C[C@H](F)C4)nc3c(F)c(-c4cc(O)cc(Cl)c4C4CC4)nc(c23)OCCC12CCCOC2]

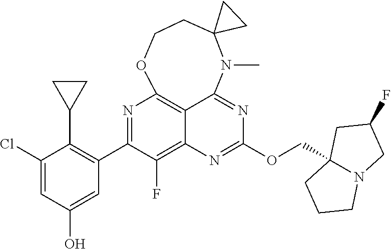 [SMILES: CN1c2nc(OC[C@@]34CCCN3C[C@H](F)C4)nc3c(F)c(-c4cc(O)cc(Cl)c4C4CC4)nc(c23)OCCC12CC2]

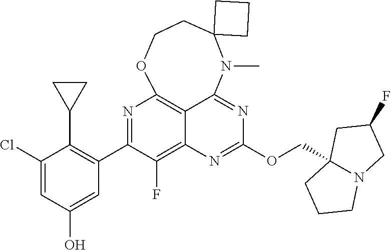 [SMILES: CN1c2nc(OC[C@@]34CCCN3C[C@H](F)C4)nc3c(F)c(-c4cc(O)cc(Cl)c4C4CC4)nc(c23)OCCC12CCC2]

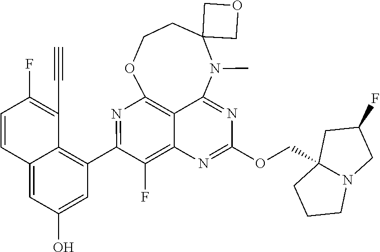 [SMILES: C#Cc1c(F)ccc2cc(O)cc(-c3nc4c5c(nc(OC[C@@]67CCCN6C[C@H](F)C7)nc5c3F)N(C)C3(CCO4)COC3)c12]

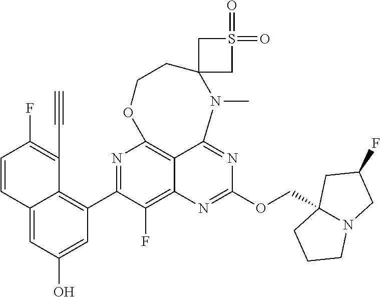 [SMILES: C#Cc1c(F)ccc2cc(O)cc(-c3nc4c5c(nc(OC[C@@]67CCCN6C[C@H](F)C7)nc5c3F)N(C)C3(CCO4)CS(=O)(=O)C3)c12]

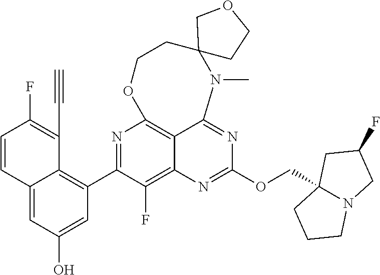 [SMILES: C#Cc1c(F)ccc2cc(O)cc(-c3nc4c5c(nc(OC[C@@]67CCCN6C[C@H](F)C7)nc5c3F)N(C)C3(CCOC3)CCO4)c12]

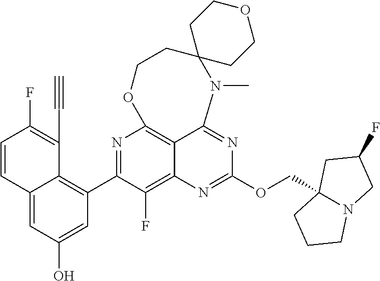 [SMILES: C#Cc1c(F)ccc2cc(O)cc(-c3nc4c5c(nc(OC[C@@]67CCCN6C[C@H](F)C7)nc5c3F)N(C)C3(CCOCC3)CCO4)c12]

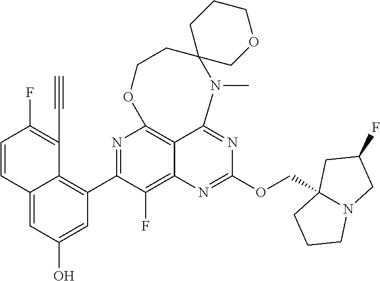 [SMILES: C#Cc1c(F)ccc2cc(O)cc(-c3nc4c5c(nc(OC[C@@]67CCCN6C[C@H](F)C7)nc5c3F)N(C)C3(CCCOC3)CCO4)c12]

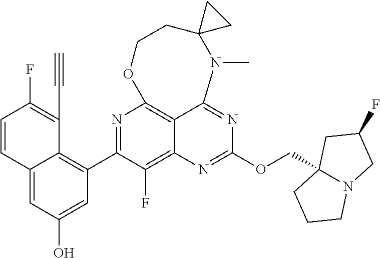 [SMILES: C#Cc1c(F)ccc2cc(O)cc(-c3nc4c5c(nc(OC[C@@]67CCCN6C[C@H](F)C7)nc5c3F)N(C)C3(CCO4)CC3)c12]

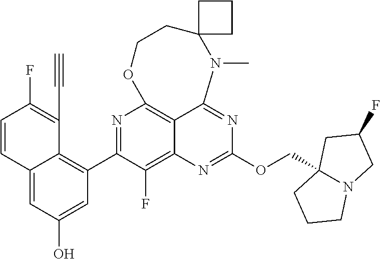 [SMILES: C#Cc1c(F)ccc2cc(O)cc(-c3nc4c5c(nc(OC[C@@]67CCCN6C[C@H](F)C7)nc5c3F)N(C)C3(CCC3)CCO4)c12]

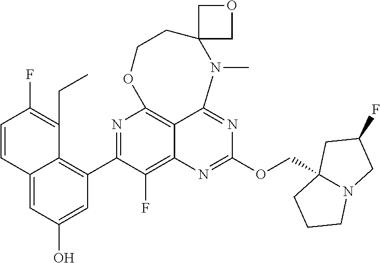 [SMILES: CCc1c(F)ccc2cc(O)cc(-c3nc4c5c(nc(OC[C@@]67CCCN6C[C@H](F)C7)nc5c3F)N(C)C3(CCO4)COC3)c12]

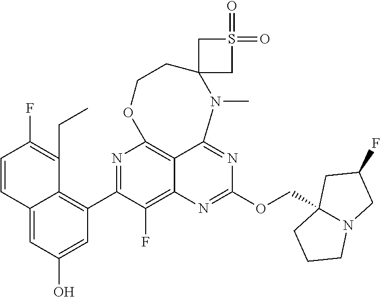 [SMILES: CCc1c(F)ccc2cc(O)cc(-c3nc4c5c(nc(OC[C@@]67CCCN6C[C@H](F)C7)nc5c3F)N(C)C3(CCO4)CS(=O)(=O)C3)c12]

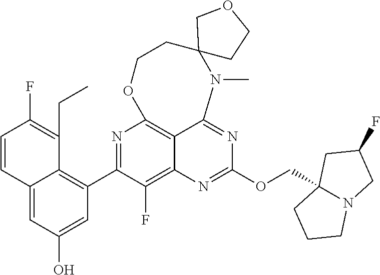 [SMILES: CCc1c(F)ccc2cc(O)cc(-c3nc4c5c(nc(OC[C@@]67CCCN6C[C@H](F)C7)nc5c3F)N(C)C3(CCOC3)CCO4)c12]

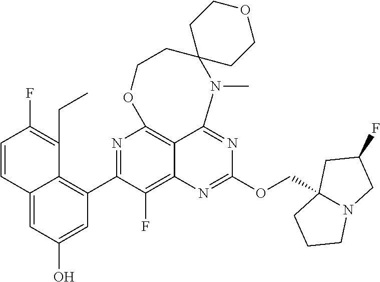 [SMILES: CCc1c(F)ccc2cc(O)cc(-c3nc4c5c(nc(OC[C@@]67CCCN6C[C@H](F)C7)nc5c3F)N(C)C3(CCOCC3)CCO4)c12]

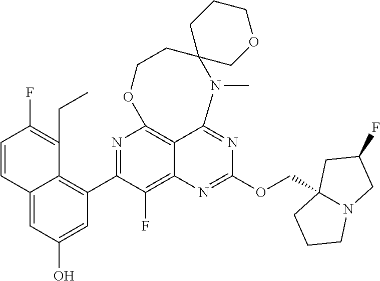 [SMILES: CCc1c(F)ccc2cc(O)cc(-c3nc4c5c(nc(OC[C@@]67CCCN6C[C@H](F)C7)nc5c3F)N(C)C3(CCCOC3)CCO4)c12]

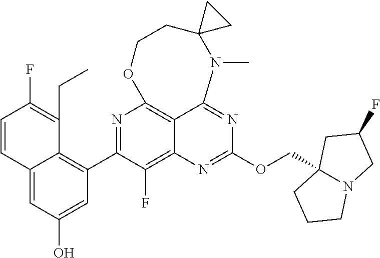 [SMILES: CCc1c(F)ccc2cc(O)cc(-c3nc4c5c(nc(OC[C@@]67CCCN6C[C@H](F)C7)nc5c3F)N(C)C3(CCO4)CC3)c12]

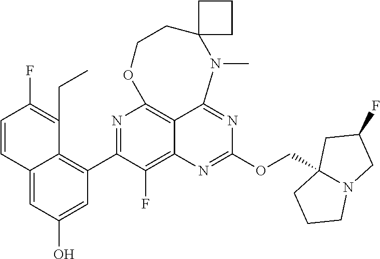 [SMILES: CCc1c(F)ccc2cc(O)cc(-c3nc4c5c(nc(OC[C@@]67CCCN6C[C@H](F)C7)nc5c3F)N(C)C3(CCC3)CCO4)c12]

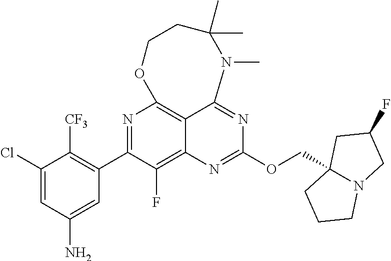 [SMILES: CN1c2nc(OC[C@@]34CCCN3C[C@H](F)C4)nc3c(F)c(-c4cc(N)cc(Cl)c4C(F)(F)F)nc(c23)OCCC1(C)C]

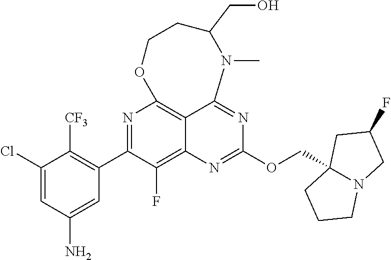 [SMILES: CN1c2nc(OC[C@@]34CCCN3C[C@H](F)C4)nc3c(F)c(-c4cc(N)cc(Cl)c4C(F)(F)F)nc(c23)OCCC1CO]

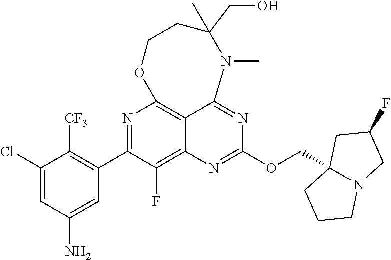 [SMILES: CN1c2nc(OC[C@@]34CCCN3C[C@H](F)C4)nc3c(F)c(-c4cc(N)cc(Cl)c4C(F)(F)F)nc(c23)OCCC1(C)CO]

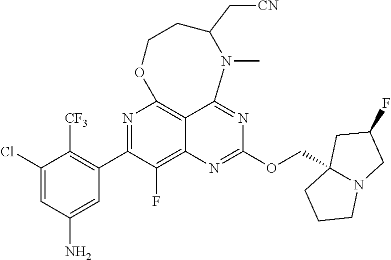 [SMILES: CN1c2nc(OC[C@@]34CCCN3C[C@H](F)C4)nc3c(F)c(-c4cc(N)cc(Cl)c4C(F)(F)F)nc(c23)OCCC1CC#N]

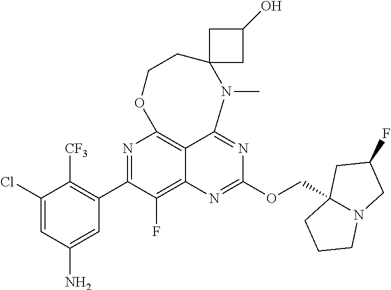 [SMILES: CN1c2nc(OC[C@@]34CCCN3C[C@H](F)C4)nc3c(F)c(-c4cc(N)cc(Cl)c4C(F)(F)F)nc(c23)OCCC12CC(O)C2]

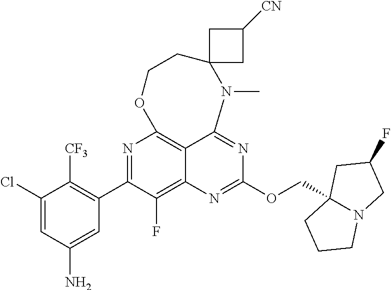 [SMILES: CN1c2nc(OC[C@@]34CCCN3C[C@H](F)C4)nc3c(F)c(-c4cc(N)cc(Cl)c4C(F)(F)F)nc(c23)OCCC12CC(C#N)C2]

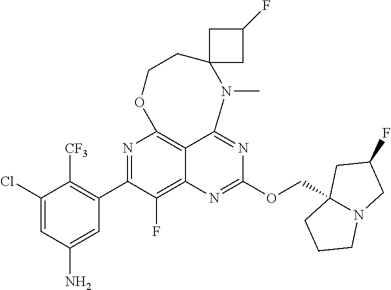 [SMILES: CN1c2nc(OC[C@@]34CCCN3C[C@H](F)C4)nc3c(F)c(-c4cc(N)cc(Cl)c4C(F)(F)F)nc(c23)OCCC12CC(F)C2]

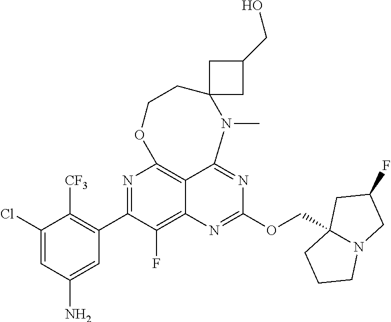 [SMILES: CN1c2nc(OC[C@@]34CCCN3C[C@H](F)C4)nc3c(F)c(-c4cc(N)cc(Cl)c4C(F)(F)F)nc(c23)OCCC12CC(CO)C2]

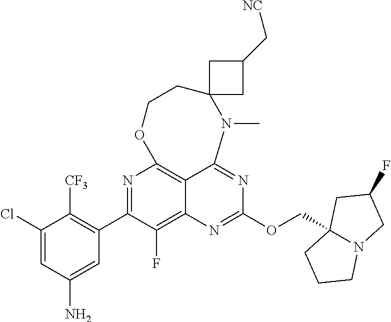 [SMILES: CN1c2nc(OC[C@@]34CCCN3C[C@H](F)C4)nc3c(F)c(-c4cc(N)cc(Cl)c4C(F)(F)F)nc(c23)OCCC12CC(CC#N)C2]

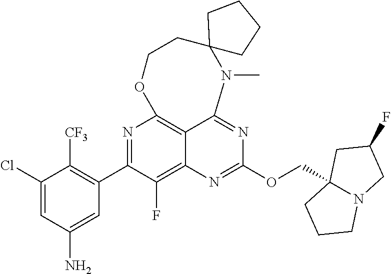 [SMILES: CN1c2nc(OC[C@@]34CCCN3C[C@H](F)C4)nc3c(F)c(-c4cc(N)cc(Cl)c4C(F)(F)F)nc(c23)OCCC12CCCC2]

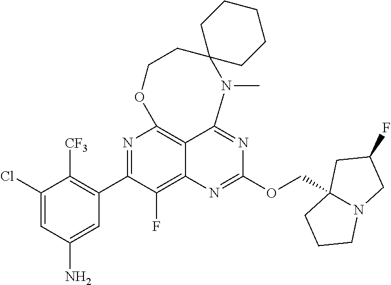 [SMILES: CN1c2nc(OC[C@@]34CCCN3C[C@H](F)C4)nc3c(F)c(-c4cc(N)cc(Cl)c4C(F)(F)F)nc(c23)OCCC12CCCCC2]

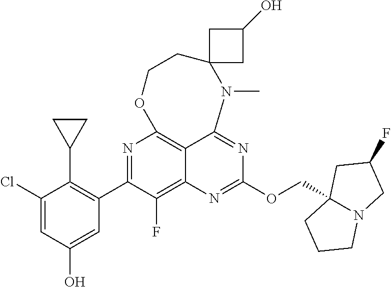 [SMILES: CN1c2nc(OC[C@@]34CCCN3C[C@H](F)C4)nc3c(F)c(-c4cc(O)cc(Cl)c4C4CC4)nc(c23)OCCC12CC(O)C2]

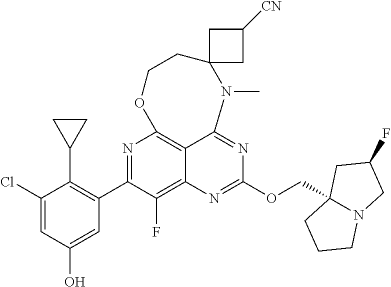 [SMILES: CN1c2nc(OC[C@@]34CCCN3C[C@H](F)C4)nc3c(F)c(-c4cc(O)cc(Cl)c4C4CC4)nc(c23)OCCC12CC(C#N)C2]

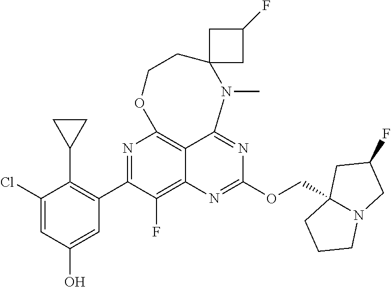 [SMILES: CN1c2nc(OC[C@@]34CCCN3C[C@H](F)C4)nc3c(F)c(-c4cc(O)cc(Cl)c4C4CC4)nc(c23)OCCC12CC(F)C2]

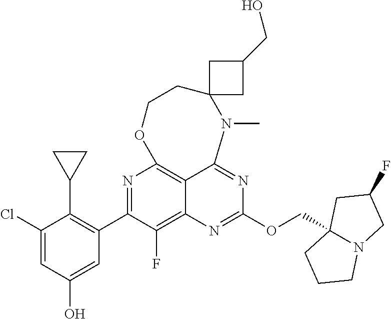 [SMILES: CN1c2nc(OC[C@@]34CCCN3C[C@H](F)C4)nc3c(F)c(-c4cc(O)cc(Cl)c4C4CC4)nc(c23)OCCC12CC(CO)C2]

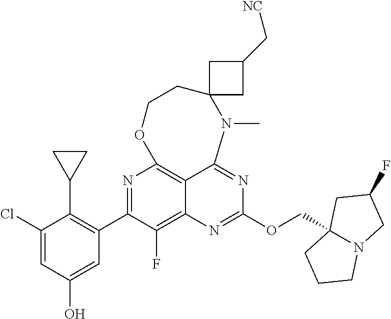 [SMILES: CN1c2nc(OC[C@@]34CCCN3C[C@H](F)C4)nc3c(F)c(-c4cc(O)cc(Cl)c4C4CC4)nc(c23)OCCC12CC(CC#N)C2]

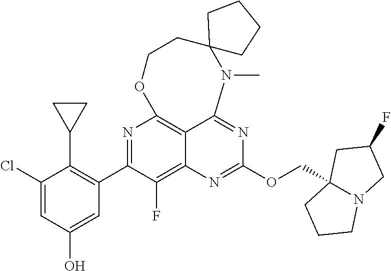 [SMILES: CN1c2nc(OC[C@@]34CCCN3C[C@H](F)C4)nc3c(F)c(-c4cc(O)cc(Cl)c4C4CC4)nc(c23)OCCC12CCCC2]

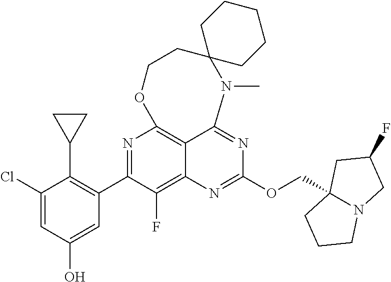 [SMILES: CN1c2nc(OC[C@@]34CCCN3C[C@H](F)C4)nc3c(F)c(-c4cc(O)cc(Cl)c4C4CC4)nc(c23)OCCC12CCCCC2]

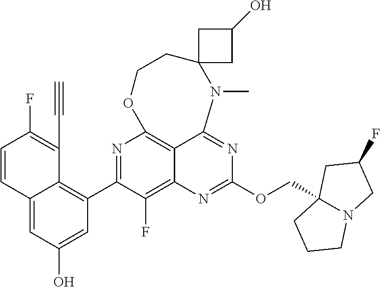 [SMILES: C#Cc1c(F)ccc2cc(O)cc(-c3nc4c5c(nc(OC[C@@]67CCCN6C[C@H](F)C7)nc5c3F)N(C)C3(CCO4)CC(O)C3)c12]

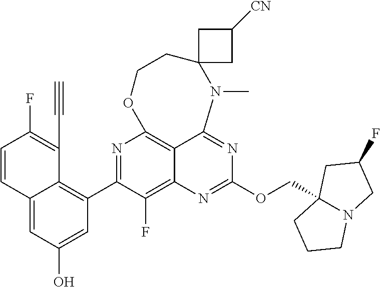 [SMILES: C#Cc1c(F)ccc2cc(O)cc(-c3nc4c5c(nc(OC[C@@]67CCCN6C[C@H](F)C7)nc5c3F)N(C)C3(CCO4)CC(C#N)C3)c12]

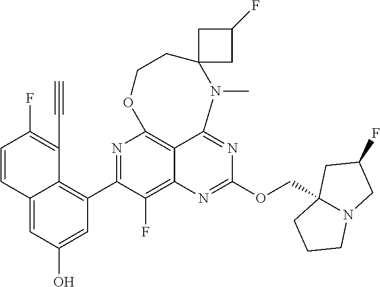 [SMILES: C#Cc1c(F)ccc2cc(O)cc(-c3nc4c5c(nc(OC[C@@]67CCCN6C[C@H](F)C7)nc5c3F)N(C)C3(CCO4)CC(F)C3)c12]

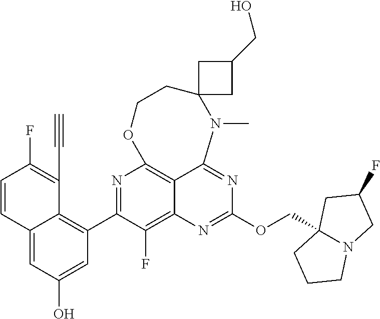 [SMILES: C#Cc1c(F)ccc2cc(O)cc(-c3nc4c5c(nc(OC[C@@]67CCCN6C[C@H](F)C7)nc5c3F)N(C)C3(CCO4)CC(CO)C3)c12]

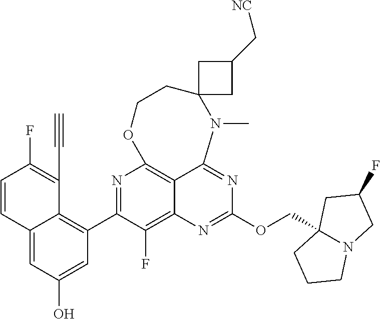 [SMILES: C#Cc1c(F)ccc2cc(O)cc(-c3nc4c5c(nc(OC[C@@]67CCCN6C[C@H](F)C7)nc5c3F)N(C)C3(CCO4)CC(CC#N)C3)c12]

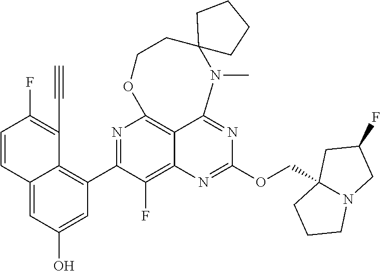 [SMILES: C#Cc1c(F)ccc2cc(O)cc(-c3nc4c5c(nc(OC[C@@]67CCCN6C[C@H](F)C7)nc5c3F)N(C)C3(CCCC3)CCO4)c12]

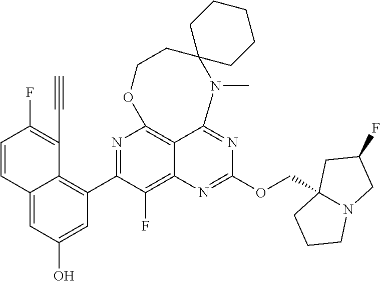 [SMILES: C#Cc1c(F)ccc2cc(O)cc(-c3nc4c5c(nc(OC[C@@]67CCCN6C[C@H](F)C7)nc5c3F)N(C)C3(CCCCC3)CCO4)c12]

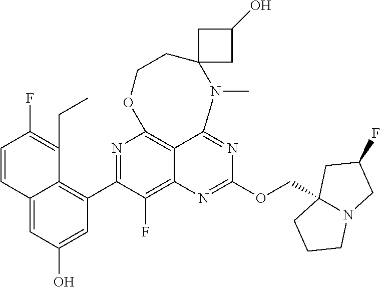 [SMILES: CCc1c(F)ccc2cc(O)cc(-c3nc4c5c(nc(OC[C@@]67CCCN6C[C@H](F)C7)nc5c3F)N(C)C3(CCO4)CC(O)C3)c12]

j

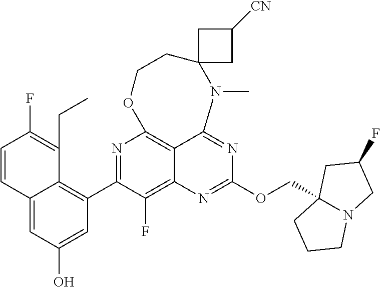 [SMILES: CCc1c(F)ccc2cc(O)cc(-c3nc4c5c(nc(OC[C@@]67CCCN6C[C@H](F)C7)nc5c3F)N(C)C3(CCO4)CC(C#N)C3)c12]

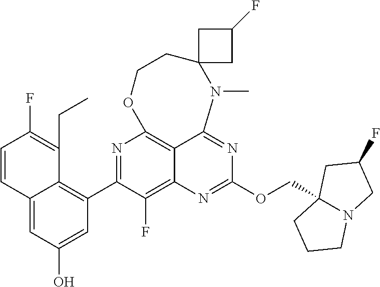 [SMILES: CCc1c(F)ccc2cc(O)cc(-c3nc4c5c(nc(OC[C@@]67CCCN6C[C@H](F)C7)nc5c3F)N(C)C3(CCO4)CC(F)C3)c12]

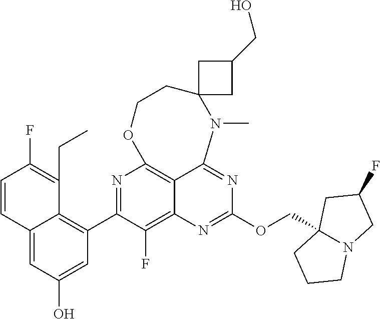 [SMILES: CCc1c(F)ccc2cc(O)cc(-c3nc4c5c(nc(OC[C@@]67CCCN6C[C@H](F)C7)nc5c3F)N(C)C3(CCO4)CC(CO)C3)c12]

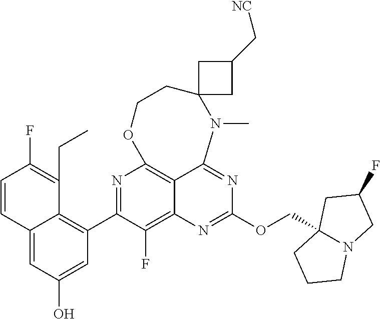 [SMILES: CCc1c(F)ccc2cc(O)cc(-c3nc4c5c(nc(OC[C@@]67CCCN6C[C@H](F)C7)nc5c3F)N(C)C3(CCO4)CC(CC#N)C3)c12]

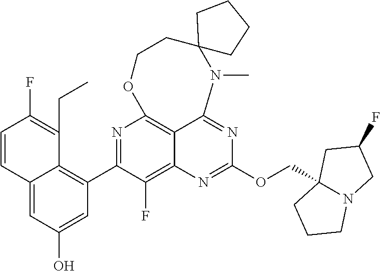 [SMILES: CCc1c(F)ccc2cc(O)cc(-c3nc4c5c(nc(OC[C@@]67CCCN6C[C@H](F)C7)nc5c3F)N(C)C3(CCCC3)CCO4)c12]

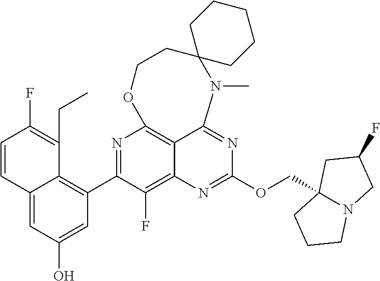 [SMILES: CCc1c(F)ccc2cc(O)cc(-c3nc4c5c(nc(OC[C@@]67CCCN6C[C@H](F)C7)nc5c3F)N(C)C3(CCCCC3)CCO4)c12]

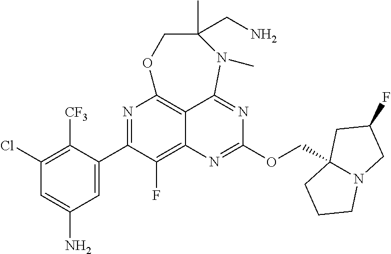 [SMILES: CN1c2nc(OC[C@@]34CCCN3C[C@H](F)C4)nc3c(F)c(-c4cc(N)cc(Cl)c4C(F)(F)F)nc(c23)OCC1(C)CN]

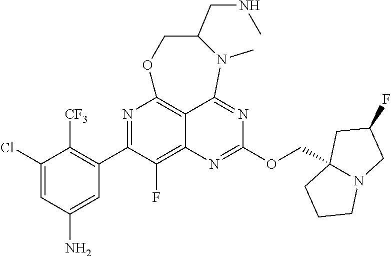 [SMILES: CNCC1COc2nc(-c3cc(N)cc(Cl)c3C(F)(F)F)c(F)c3nc(OC[C@@]45CCCN4C[C@H](F)C5)nc(c23)N1C]

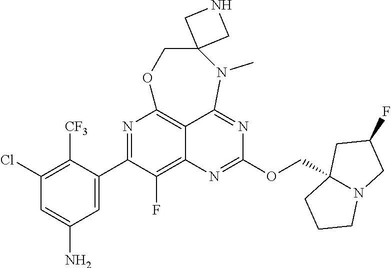 [SMILES: CN1c2nc(OC[C@@]34CCCN3C[C@H](F)C4)nc3c(F)c(-c4cc(N)cc(Cl)c4C(F)(F)F)nc(c23)OCC12CNC2]

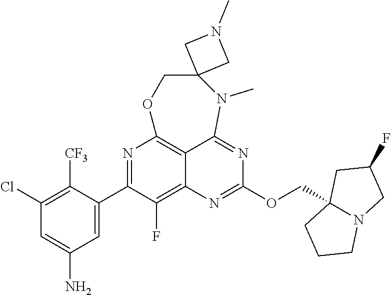 [SMILES: CN1CC2(COc3nc(-c4cc(N)cc(Cl)c4C(F)(F)F)c(F)c4nc(OC[C@@]56CCCN5C[C@H](F)C6)nc(c34)N2C)C1]

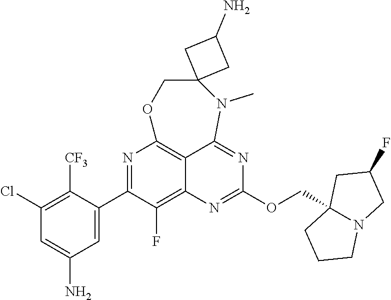 [SMILES: CN1c2nc(OC[C@@]34CCCN3C[C@H](F)C4)nc3c(F)c(-c4cc(N)cc(Cl)c4C(F)(F)F)nc(c23)OCC12CC(N)C2]

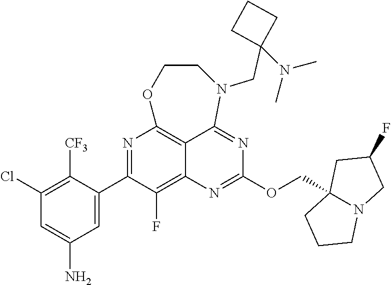 [SMILES: CN(C)C1(CN2CCOc3nc(-c4cc(N)cc(Cl)c4C(F)(F)F)c(F)c4nc(OC[C@@]56CCCN5C[C@H](F)C6)nc2c34)CCC1]

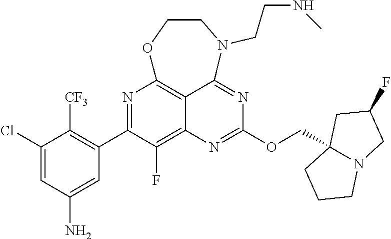 [SMILES: CNCCN1CCOc2nc(-c3cc(N)cc(Cl)c3C(F)(F)F)c(F)c3nc(OC[C@@]45CCCN4C[C@H](F)C5)nc1c23]

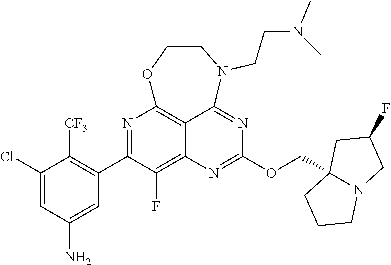 [SMILES: CN(C)CCN1CCOc2nc(-c3cc(N)cc(Cl)c3C(F)(F)F)c(F)c3nc(OC[C@@]45CCCN4C[C@H](F)C5)nc1c23]

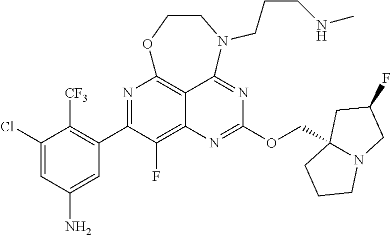 [SMILES: CNCCCN1CCOc2nc(-c3cc(N)cc(Cl)c3C(F)(F)F)c(F)c3nc(OC[C@@]45CCCN4C[C@H](F)C5)nc1c23]

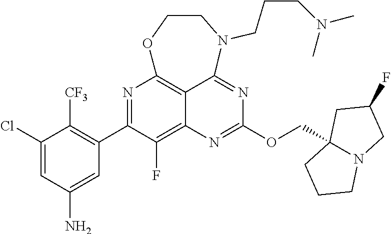 [SMILES: CN(C)CCCN1CCOc2nc(-c3cc(N)cc(Cl)c3C(F)(F)F)c(F)c3nc(OC[C@@]45CCCN4C[C@H](F)C5)nc1c23]

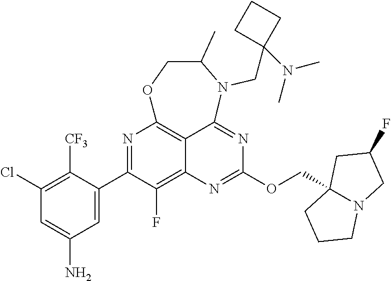 [SMILES: CC1COc2nc(-c3cc(N)cc(Cl)c3C(F)(F)F)c(F)c3nc(OC[C@@]45CCCN4C[C@H](F)C5)nc(c23)N1CC1(N(C)C)CCC1]

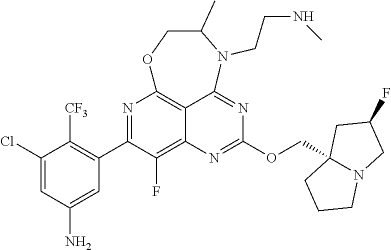 [SMILES: CNCCN1c2nc(OC[C@@]34CCCN3C[C@H](F)C4)nc3c(F)c(-c4cc(N)cc(Cl)c4C(F)(F)F)nc(c23)OCC1C]

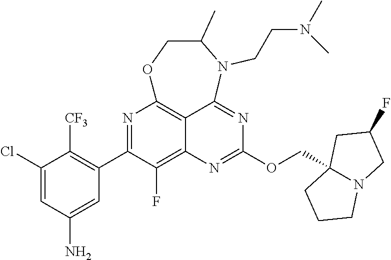 [SMILES: CC1COc2nc(-c3cc(N)cc(Cl)c3C(F)(F)F)c(F)c3nc(OC[C@@]45CCCN4C[C@H](F)C5)nc(c23)N1CCN(C)C]

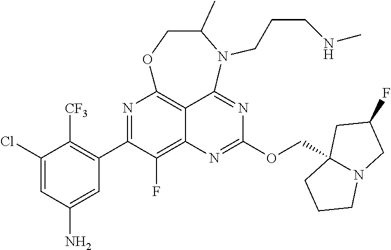 [SMILES: CNCCCN1c2nc(OC[C@@]34CCCN3C[C@H](F)C4)nc3c(F)c(-c4cc(N)cc(Cl)c4C(F)(F)F)nc(c23)OCC1C]

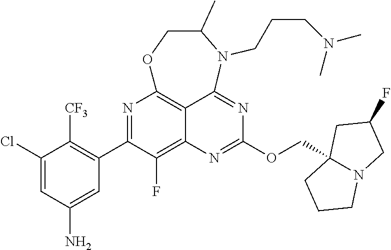 [SMILES: CC1COc2nc(-c3cc(N)cc(Cl)c3C(F)(F)F)c(F)c3nc(OC[C@@]45CCCN4C[C@H](F)C5)nc(c23)N1CCCN(C)C]

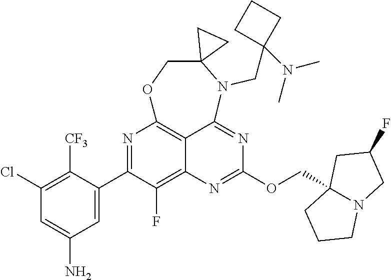 [SMILES: CN(C)C1(CN2c3nc(OC[C@@]45CCCN4C[C@H](F)C5)nc4c(F)c(-c5cc(N)cc(Cl)c5C(F)(F)F)nc(c34)OCC23CC3)CCC1]

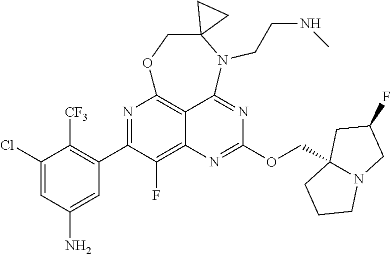 [SMILES: CNCCN1c2nc(OC[C@@]34CCCN3C[C@H](F)C4)nc3c(F)c(-c4cc(N)cc(Cl)c4C(F)(F)F)nc(c23)OCC12CC2]

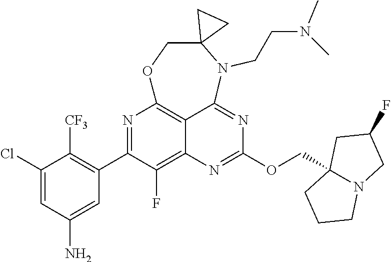 [SMILES: CN(C)CCN1c2nc(OC[C@@]34CCCN3C[C@H](F)C4)nc3c(F)c(-c4cc(N)cc(Cl)c4C(F)(F)F)nc(c23)OCC12CC2]

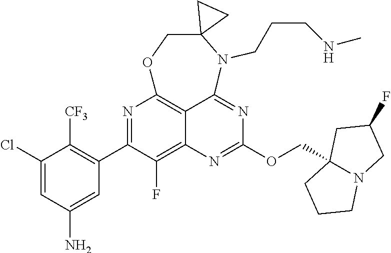 [SMILES: CNCCCN1c2nc(OC[C@@]34CCCN3C[C@H](F)C4)nc3c(F)c(-c4cc(N)cc(Cl)c4C(F)(F)F)nc(c23)OCC12CC2]

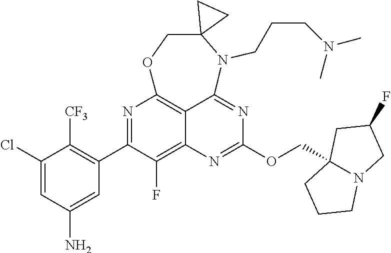 [SMILES: CN(C)CCCN1c2nc(OC[C@@]34CCCN3C[C@H](F)C4)nc3c(F)c(-c4cc(N)cc(Cl)c4C(F)(F)F)nc(c23)OCC12CC2]

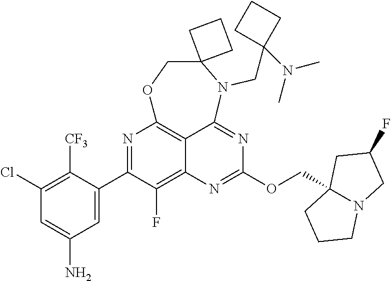 [SMILES: CN(C)C1(CN2c3nc(OC[C@@]45CCCN4C[C@H](F)C5)nc4c(F)c(-c5cc(N)cc(Cl)c5C(F)(F)F)nc(c34)OCC23CCC3)CCC1]

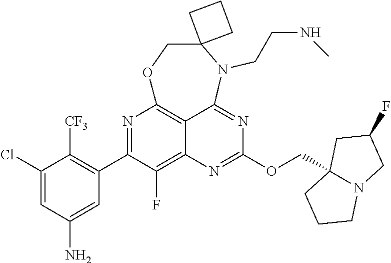 [SMILES: CNCCN1c2nc(OC[C@@]34CCCN3C[C@H](F)C4)nc3c(F)c(-c4cc(N)cc(Cl)c4C(F)(F)F)nc(c23)OCC12CCC2]

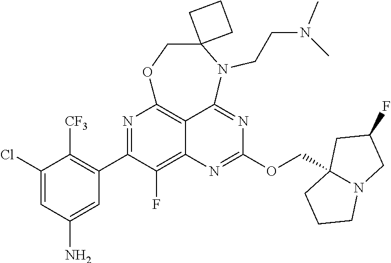 [SMILES: CN(C)CCN1c2nc(OC[C@@]34CCCN3C[C@H](F)C4)nc3c(F)c(-c4cc(N)cc(Cl)c4C(F)(F)F)nc(c23)OCC12CCC2]

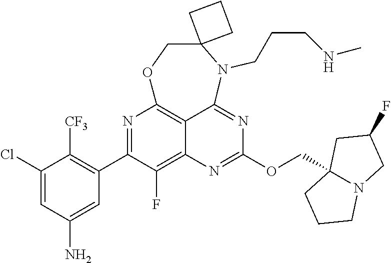 [SMILES: CNCCCN1c2nc(OC[C@@]34CCCN3C[C@H](F)C4)nc3c(F)c(-c4cc(N)cc(Cl)c4C(F)(F)F)nc(c23)OCC12CCC2]

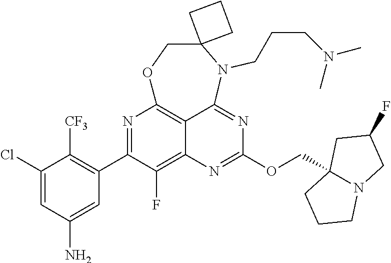 [SMILES: CN(C)CCCN1c2nc(OC[C@@]34CCCN3C[C@H](F)C4)nc3c(F)c(-c4cc(N)cc(Cl)c4C(F)(F)F)nc(c23)OCC12CCC2]

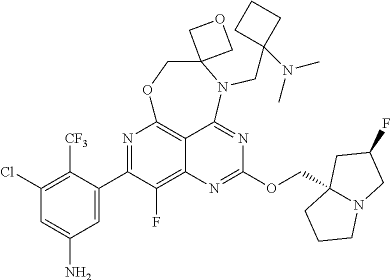 [SMILES: CN(C)C1(CN2c3nc(OC[C@@]45CCCN4C[C@H](F)C5)nc4c(F)c(-c5cc(N)cc(Cl)c5C(F)(F)F)nc(c34)OCC23COC3)CCC1]

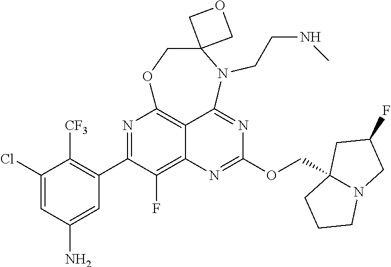 [SMILES: CNCCN1c2nc(OC[C@@]34CCCN3C[C@H](F)C4)nc3c(F)c(-c4cc(N)cc(Cl)c4C(F)(F)F)nc(c23)OCC12COC2]

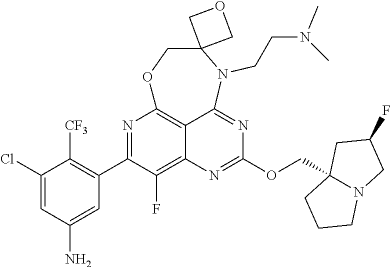 [SMILES: CN(C)CCN1c2nc(OC[C@@]34CCCN3C[C@H](F)C4)nc3c(F)c(-c4cc(N)cc(Cl)c4C(F)(F)F)nc(c23)OCC12COC2]

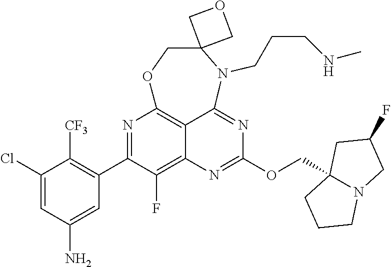 [SMILES: CNCCCN1c2nc(OC[C@@]34CCCN3C[C@H](F)C4)nc3c(F)c(-c4cc(N)cc(Cl)c4C(F)(F)F)nc(c23)OCC12COC2]

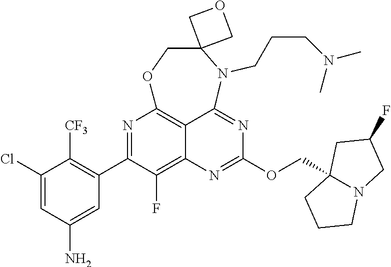 [SMILES: CN(C)CCCN1c2nc(OC[C@@]34CCCN3C[C@H](F)C4)nc3c(F)c(-c4cc(N)cc(Cl)c4C(F)(F)F)nc(c23)OCC12COC2]

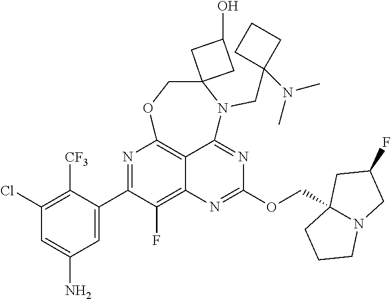 [SMILES: CN(C)C1(CN2c3nc(OC[C@@]45CCCN4C[C@H](F)C5)nc4c(F)c(-c5cc(N)cc(Cl)c5C(F)(F)F)nc(c34)OCC23CC(O)C3)CCC1]

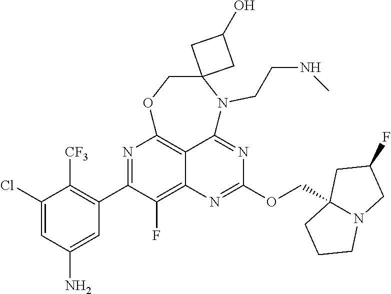 [SMILES: CNCCN1c2nc(OC[C@@]34CCCN3C[C@H](F)C4)nc3c(F)c(-c4cc(N)cc(Cl)c4C(F)(F)F)nc(c23)OCC12CC(O)C2]

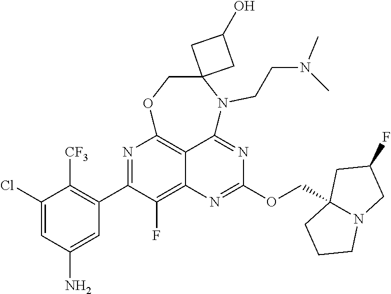 [SMILES: CN(C)CCN1c2nc(OC[C@@]34CCCN3C[C@H](F)C4)nc3c(F)c(-c4cc(N)cc(Cl)c4C(F)(F)F)nc(c23)OCC12CC(O)C2]

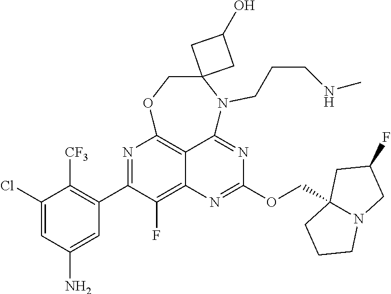 [SMILES: CNCCCN1c2nc(OC[C@@]34CCCN3C[C@H](F)C4)nc3c(F)c(-c4cc(N)cc(Cl)c4C(F)(F)F)nc(c23)OCC12CC(O)C2]

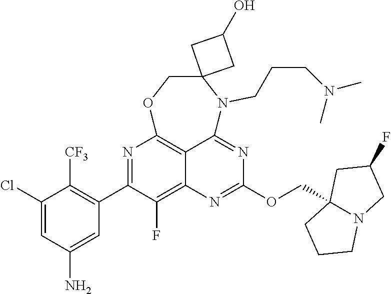 [SMILES: CN(C)CCCN1c2nc(OC[C@@]34CCCN3C[C@H](F)C4)nc3c(F)c(-c4cc(N)cc(Cl)c4C(F)(F)F)nc(c23)OCC12CC(O)C2]

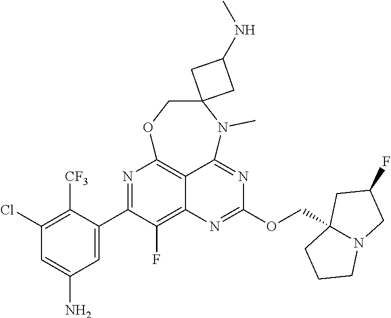 [SMILES: CNC1CC2(COc3nc(-c4cc(N)cc(Cl)c4C(F)(F)F)c(F)c4nc(OC[C@@]56CCCN5C[C@H](F)C6)nc(c34)N2C)C1]

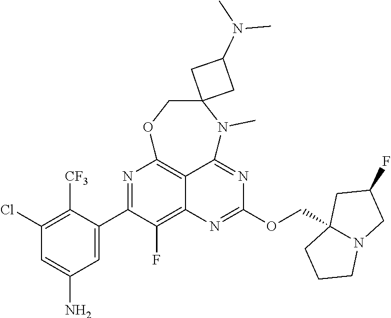 [SMILES: CN(C)C1CC2(COc3nc(-c4cc(N)cc(Cl)c4C(F)(F)F)c(F)c4nc(OC[C@@]56CCCN5C[C@H](F)C6)nc(c34)N2C)C1]

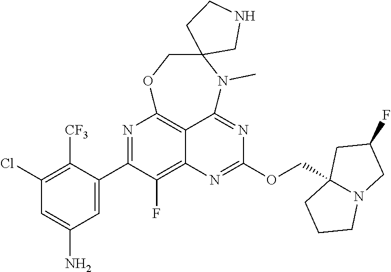 [SMILES: CN1c2nc(OC[C@@]34CCCN3C[C@H](F)C4)nc3c(F)c(-c4cc(N)cc(Cl)c4C(F)(F)F)nc(c23)OCC12CCNC2]

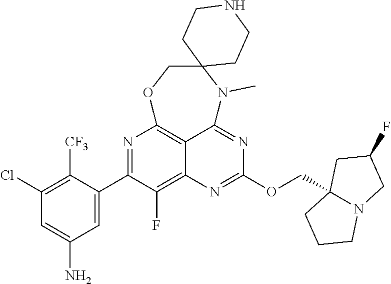 [SMILES: CN1c2nc(OC[C@@]34CCCN3C[C@H](F)C4)nc3c(F)c(-c4cc(N)cc(Cl)c4C(F)(F)F)nc(c23)OCC12CCNCC2]

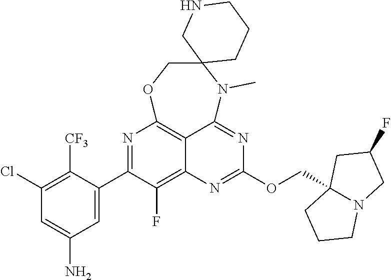 [SMILES: CN1c2nc(OC[C@@]34CCCN3C[C@H](F)C4)nc3c(F)c(-c4cc(N)cc(Cl)c4C(F)(F)F)nc(c23)OCC12CCCNC2]

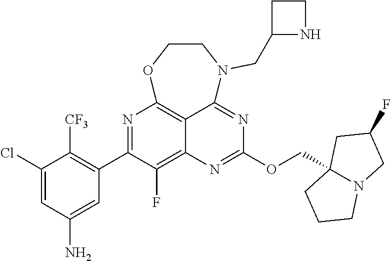 [SMILES: Nc1cc(Cl)c(C(F)(F)F)c(-c2nc3c4c(nc(OC[C@@]56CCCN5C[C@H](F)C6)nc4c2F)N(CC2CCN2)CCO3)c1]

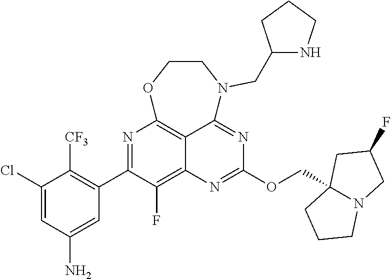 [SMILES: Nc1cc(Cl)c(C(F)(F)F)c(-c2nc3c4c(nc(OC[C@@]56CCCN5C[C@H](F)C6)nc4c2F)N(CC2CCCN2)CCO3)c1]

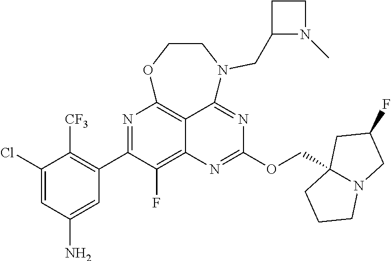 [SMILES: CN1CCC1CN1CCOc2nc(-c3cc(N)cc(Cl)c3C(F)(F)F)c(F)c3nc(OC[C@@]45CCCN4C[C@H](F)C5)nc1c23]

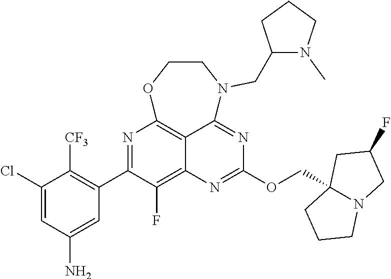 [SMILES: CN1CCCC1CN1CCOc2nc(-c3cc(N)cc(Cl)c3C(F)(F)F)c(F)c3nc(OC[C@@]45CCCN4C[C@H](F)C5)nc1c23]

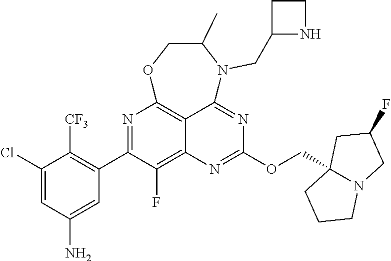 [SMILES: CC1COc2nc(-c3cc(N)cc(Cl)c3C(F)(F)F)c(F)c3nc(OC[C@@]45CCCN4C[C@H](F)C5)nc(c23)N1CC1CCN1]

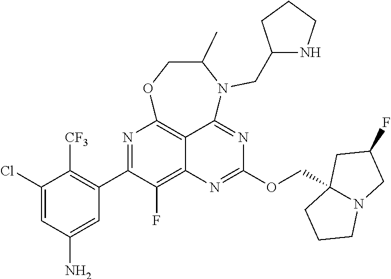 [SMILES: CC1COc2nc(-c3cc(N)cc(Cl)c3C(F)(F)F)c(F)c3nc(OC[C@@]45CCCN4C[C@H](F)C5)nc(c23)N1CC1CCCN1]

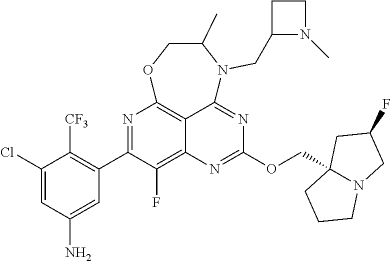 [SMILES: CC1COc2nc(-c3cc(N)cc(Cl)c3C(F)(F)F)c(F)c3nc(OC[C@@]45CCCN4C[C@H](F)C5)nc(c23)N1CC1CCN1C]

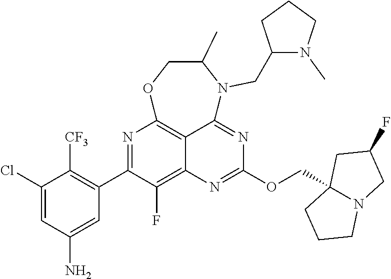 [SMILES: CC1COc2nc(-c3cc(N)cc(Cl)c3C(F)(F)F)c(F)c3nc(OC[C@@]45CCCN4C[C@H](F)C5)nc(c23)N1CC1CCCN1C]

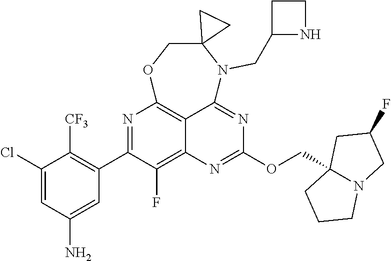 [SMILES: Nc1cc(Cl)c(C(F)(F)F)c(-c2nc3c4c(nc(OC[C@@]56CCCN5C[C@H](F)C6)nc4c2F)N(CC2CCN2)C2(CC2)CO3)c1]

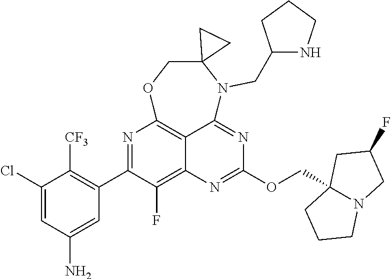 [SMILES: Nc1cc(Cl)c(C(F)(F)F)c(-c2nc3c4c(nc(OC[C@@]56CCCN5C[C@H](F)C6)nc4c2F)N(CC2CCCN2)C2(CC2)CO3)c1]

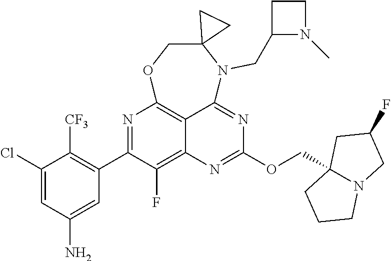 [SMILES: CN1CCC1CN1c2nc(OC[C@@]34CCCN3C[C@H](F)C4)nc3c(F)c(-c4cc(N)cc(Cl)c4C(F)(F)F)nc(c23)OCC12CC2]

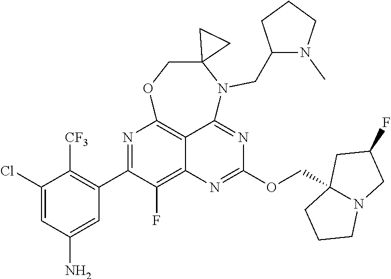 [SMILES: CN1CCCC1CN1c2nc(OC[C@@]34CCCN3C[C@H](F)C4)nc3c(F)c(-c4cc(N)cc(Cl)c4C(F)(F)F)nc(c23)OCC12CC2]

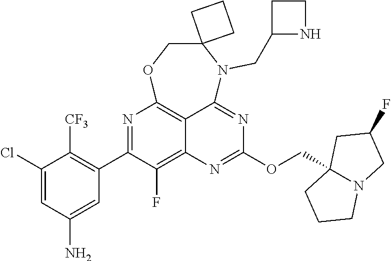 [SMILES: Nc1cc(Cl)c(C(F)(F)F)c(-c2nc3c4c(nc(OC[C@@]56CCCN5C[C@H](F)C6)nc4c2F)N(CC2CCN2)C2(CCC2)CO3)c1]

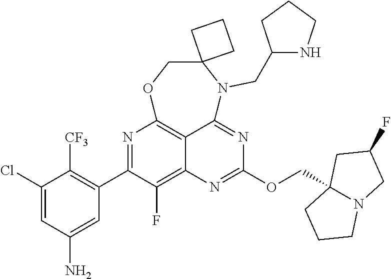 [SMILES: Nc1cc(Cl)c(C(F)(F)F)c(-c2nc3c4c(nc(OC[C@@]56CCCN5C[C@H](F)C6)nc4c2F)N(CC2CCCN2)C2(CCC2)CO3)c1]

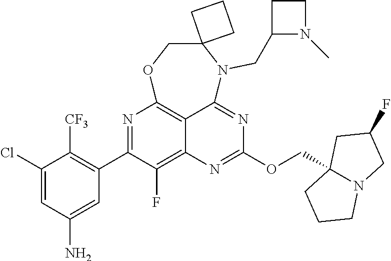 [SMILES: CN1CCC1CN1c2nc(OC[C@@]34CCCN3C[C@H](F)C4)nc3c(F)c(-c4cc(N)cc(Cl)c4C(F)(F)F)nc(c23)OCC12CCC2]

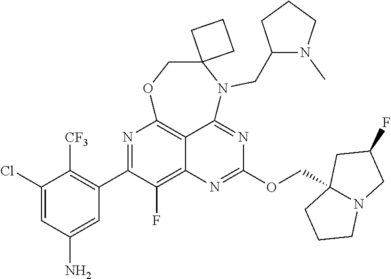 [SMILES: CN1CCCC1CN1c2nc(OC[C@@]34CCCN3C[C@H](F)C4)nc3c(F)c(-c4cc(N)cc(Cl)c4C(F)(F)F)nc(c23)OCC12CCC2]

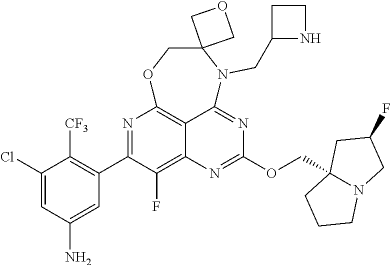 [SMILES: Nc1cc(Cl)c(C(F)(F)F)c(-c2nc3c4c(nc(OC[C@@]56CCCN5C[C@H](F)C6)nc4c2F)N(CC2CCN2)C2(COC2)CO3)c1]

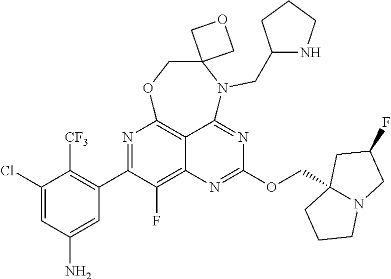 [SMILES: Nc1cc(Cl)c(C(F)(F)F)c(-c2nc3c4c(nc(OC[C@@]56CCCN5C[C@H](F)C6)nc4c2F)N(CC2CCCN2)C2(COC2)CO3)c1]

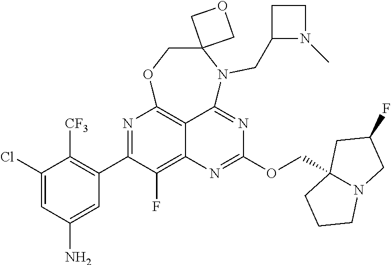 [SMILES: CN1CCC1CN1c2nc(OC[C@@]34CCCN3C[C@H](F)C4)nc3c(F)c(-c4cc(N)cc(Cl)c4C(F)(F)F)nc(c23)OCC12COC2]

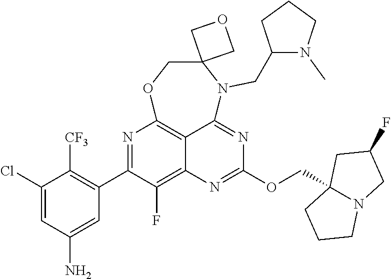 [SMILES: CN1CCCC1CN1c2nc(OC[C@@]34CCCN3C[C@H](F)C4)nc3c(F)c(-c4cc(N)cc(Cl)c4C(F)(F)F)nc(c23)OCC12COC2]

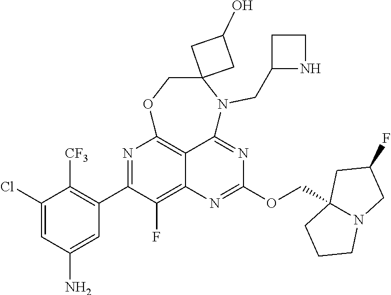 [SMILES: Nc1cc(Cl)c(C(F)(F)F)c(-c2nc3c4c(nc(OC[C@@]56CCCN5C[C@H](F)C6)nc4c2F)N(CC2CCN2)C2(CO3)CC(O)C2)c1]

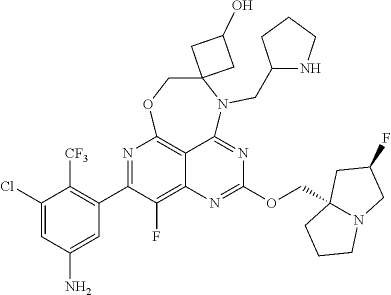 [SMILES: Nc1cc(Cl)c(C(F)(F)F)c(-c2nc3c4c(nc(OC[C@@]56CCCN5C[C@H](F)C6)nc4c2F)N(CC2CCCN2)C2(CO3)CC(O)C2)c1]

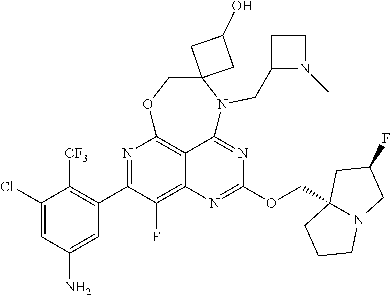 [SMILES: CN1CCC1CN1c2nc(OC[C@@]34CCCN3C[C@H](F)C4)nc3c(F)c(-c4cc(N)cc(Cl)c4C(F)(F)F)nc(c23)OCC12CC(O)C2]

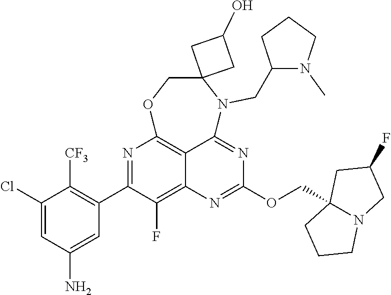 [SMILES: CN1CCCC1CN1c2nc(OC[C@@]34CCCN3C[C@H](F)C4)nc3c(F)c(-c4cc(N)cc(Cl)c4C(F)(F)F)nc(c23)OCC12CC(O)C2]

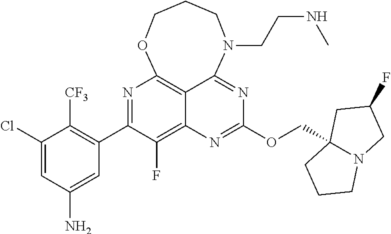 [SMILES: CNCCN1CCCOc2nc(-c3cc(N)cc(Cl)c3C(F)(F)F)c(F)c3nc(OC[C@@]45CCCN4C[C@H](F)C5)nc1c23]

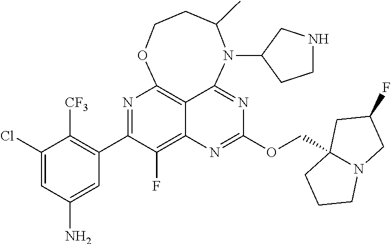 [SMILES: CC1CCOc2nc(-c3cc(N)cc(Cl)c3C(F)(F)F)c(F)c3nc(OC[C@@]45CCCN4C[C@H](F)C5)nc(c23)N1C1CCNC1]

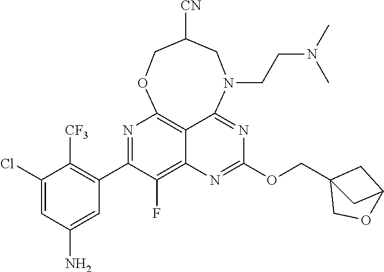 [SMILES: CN(C)CCN1CC(C#N)COc2nc(-c3cc(N)cc(Cl)c3C(F)(F)F)c(F)c3nc(OCC45COC(C4)C5)nc1c23]

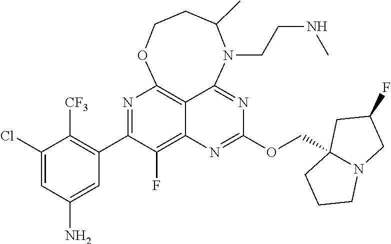 [SMILES: CNCCN1c2nc(OC[C@@]34CCCN3C[C@H](F)C4)nc3c(F)c(-c4cc(N)cc(Cl)c4C(F)(F)F)nc(c23)OCCC1C]

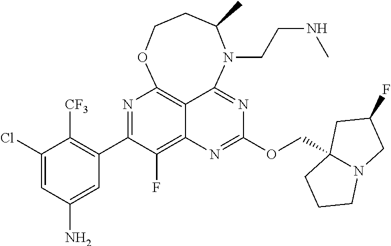 [SMILES: CNCCN1c2nc(OC[C@@]34CCCN3C[C@H](F)C4)nc3c(F)c(-c4cc(N)cc(Cl)c4C(F)(F)F)nc(c23)OCC[C@H]1C]

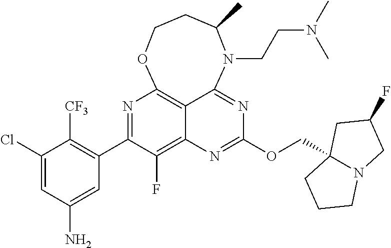 [SMILES: C[C@@H]1CCOc2nc(-c3cc(N)cc(Cl)c3C(F)(F)F)c(F)c3nc(OC[C@@]45CCCN4C[C@H](F)C5)nc(c23)N1CCN(C)C]

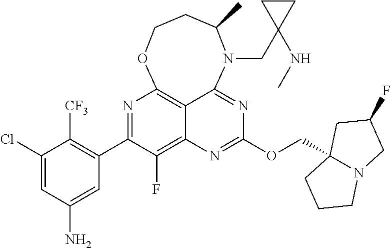 [SMILES: CNC1(CN2c3nc(OC[C@@]45CCCN4C[C@H](F)C5)nc4c(F)c(-c5cc(N)cc(Cl)c5C(F)(F)F)nc(c34)OCC[C@H]2C)CC1]

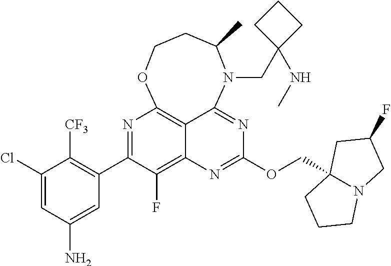 [SMILES: CNC1(CN2c3nc(OC[C@@]45CCCN4C[C@H](F)C5)nc4c(F)c(-c5cc(N)cc(Cl)c5C(F)(F)F)nc(c34)OCC[C@H]2C)CCC1]

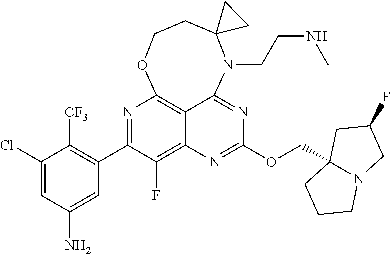 [SMILES: CNCCN1c2nc(OC[C@@]34CCCN3C[C@H](F)C4)nc3c(F)c(-c4cc(N)cc(Cl)c4C(F)(F)F)nc(c23)OCCC12CC2]

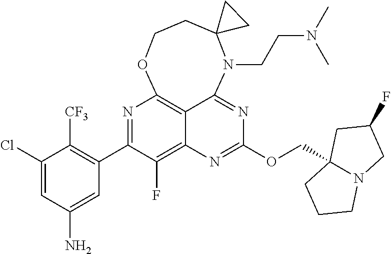 [SMILES: CN(C)CCN1c2nc(OC[C@@]34CCCN3C[C@H](F)C4)nc3c(F)c(-c4cc(N)cc(Cl)c4C(F)(F)F)nc(c23)OCCC12CC2]

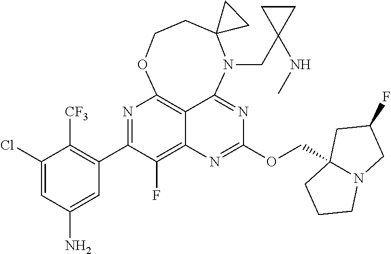 [SMILES: CNC1(CN2c3nc(OC[C@@]45CCCN4C[C@H](F)C5)nc4c(F)c(-c5cc(N)cc(Cl)c5C(F)(F)F)nc(c34)OCCC23CC3)CC1]

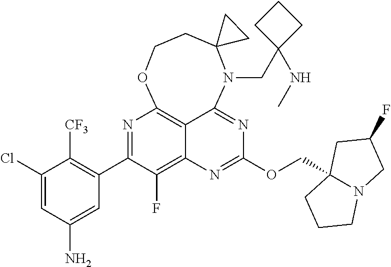 [SMILES: CNC1(CN2c3nc(OC[C@@]45CCCN4C[C@H](F)C5)nc4c(F)c(-c5cc(N)cc(Cl)c5C(F)(F)F)nc(c34)OCCC23CC3)CCC1]

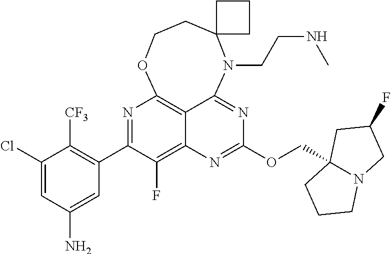 [SMILES: CNCCN1c2nc(OC[C@@]34CCCN3C[C@H](F)C4)nc3c(F)c(-c4cc(N)cc(Cl)c4C(F)(F)F)nc(c23)OCCC12CCC2]

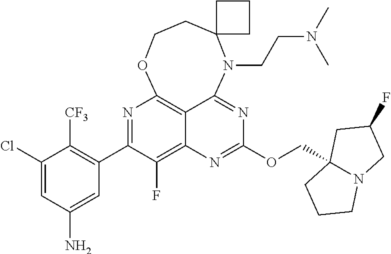 [SMILES: CN(C)CCN1c2nc(OC[C@@]34CCCN3C[C@H](F)C4)nc3c(F)c(-c4cc(N)cc(Cl)c4C(F)(F)F)nc(c23)OCCC12CCC2]

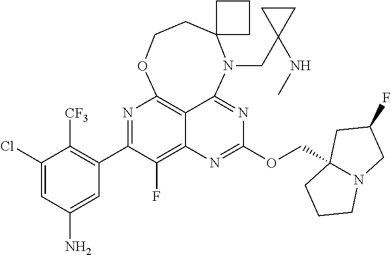 [SMILES: CNC1(CN2c3nc(OC[C@@]45CCCN4C[C@H](F)C5)nc4c(F)c(-c5cc(N)cc(Cl)c5C(F)(F)F)nc(c34)OCCC23CCC3)CC1]

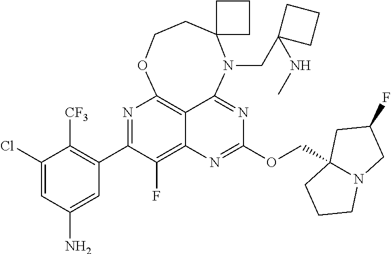 [SMILES: CNC1(CN2c3nc(OC[C@@]45CCCN4C[C@H](F)C5)nc4c(F)c(-c5cc(N)cc(Cl)c5C(F)(F)F)nc(c34)OCCC23CCC3)CCC1]

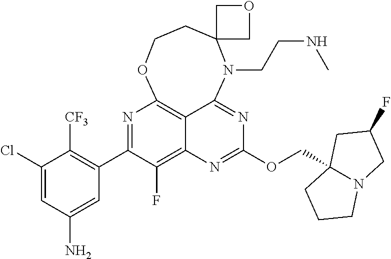 [SMILES: CNCCN1c2nc(OC[C@@]34CCCN3C[C@H](F)C4)nc3c(F)c(-c4cc(N)cc(Cl)c4C(F)(F)F)nc(c23)OCCC12COC2]

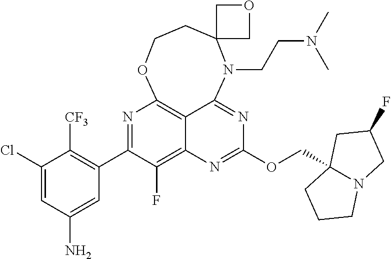 [SMILES: CN(C)CCN1c2nc(OC[C@@]34CCCN3C[C@H](F)C4)nc3c(F)c(-c4cc(N)cc(Cl)c4C(F)(F)F)nc(c23)OCCC12COC2]

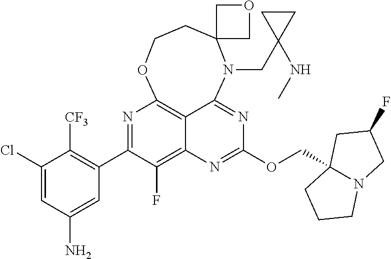 [SMILES: CNC1(CN2c3nc(OC[C@@]45CCCN4C[C@H](F)C5)nc4c(F)c(-c5cc(N)cc(Cl)c5C(F)(F)F)nc(c34)OCCC23COC3)CC1]

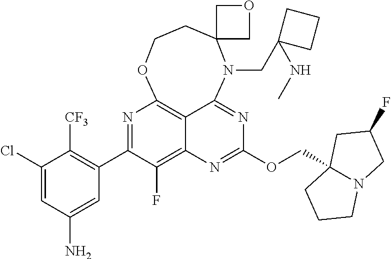 [SMILES: CNC1(CN2c3nc(OC[C@@]45CCCN4C[C@H](F)C5)nc4c(F)c(-c5cc(N)cc(Cl)c5C(F)(F)F)nc(c34)OCCC23COC3)CCC1]

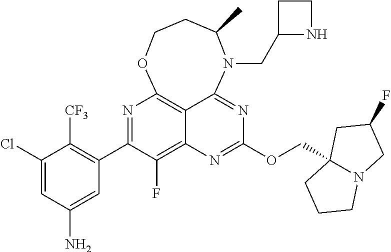 [SMILES: C[C@@H]1CCOc2nc(-c3cc(N)cc(Cl)c3C(F)(F)F)c(F)c3nc(OC[C@@]45CCCN4C[C@H](F)C5)nc(c23)N1CC1CCN1]

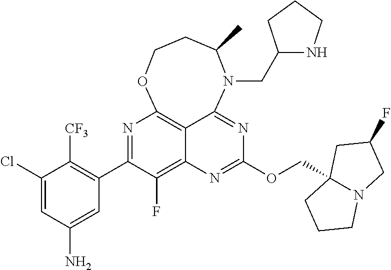 [SMILES: C[C@@H]1CCOc2nc(-c3cc(N)cc(Cl)c3C(F)(F)F)c(F)c3nc(OC[C@@]45CCCN4C[C@H](F)C5)nc(c23)N1CC1CCCN1]

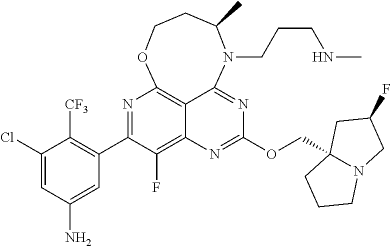 [SMILES: CNCCCN1c2nc(OC[C@@]34CCCN3C[C@H](F)C4)nc3c(F)c(-c4cc(N)cc(Cl)c4C(F)(F)F)nc(c23)OCC[C@H]1C]

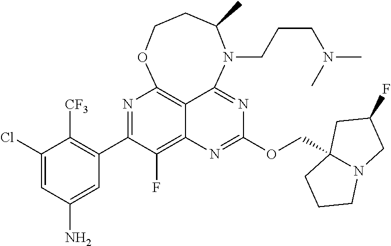 [SMILES: C[C@@H]1CCOc2nc(-c3cc(N)cc(Cl)c3C(F)(F)F)c(F)c3nc(OC[C@@]45CCCN4C[C@H](F)C5)nc(c23)N1CCCN(C)C]

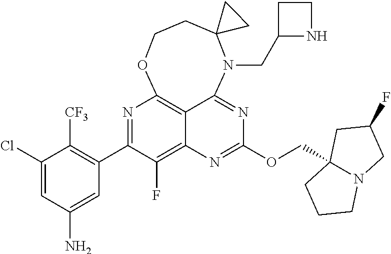 [SMILES: Nc1cc(Cl)c(C(F)(F)F)c(-c2nc3c4c(nc(OC[C@@]56CCCN5C[C@H](F)C6)nc4c2F)N(CC2CCN2)C2(CCO3)CC2)c1]

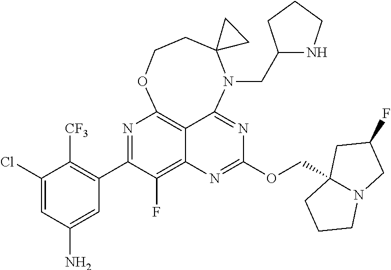 [SMILES: Nc1cc(Cl)c(C(F)(F)F)c(-c2nc3c4c(nc(OC[C@@]56CCCN5C[C@H](F)C6)nc4c2F)N(CC2CCCN2)C2(CCO3)CC2)c1]

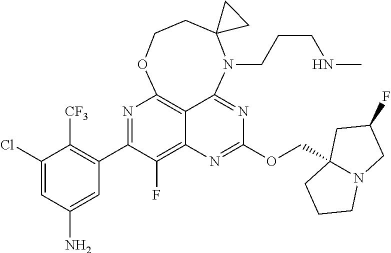 [SMILES: CNCCCN1c2nc(OC[C@@]34CCCN3C[C@H](F)C4)nc3c(F)c(-c4cc(N)cc(Cl)c4C(F)(F)F)nc(c23)OCCC12CC2]

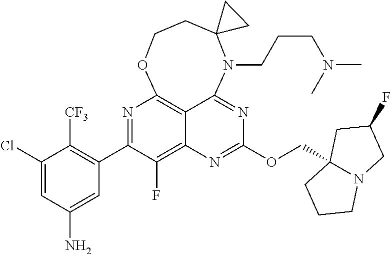 [SMILES: CN(C)CCCN1c2nc(OC[C@@]34CCCN3C[C@H](F)C4)nc3c(F)c(-c4cc(N)cc(Cl)c4C(F)(F)F)nc(c23)OCCC12CC2]

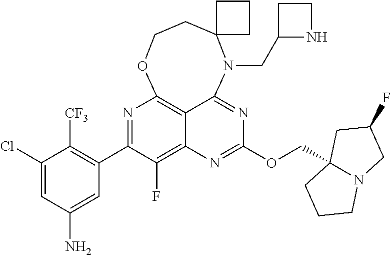 [SMILES: Nc1cc(Cl)c(C(F)(F)F)c(-c2nc3c4c(nc(OC[C@@]56CCCN5C[C@H](F)C6)nc4c2F)N(CC2CCN2)C2(CCC2)CCO3)c1]

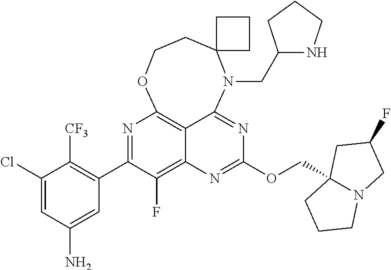 [SMILES: Nc1cc(Cl)c(C(F)(F)F)c(-c2nc3c4c(nc(OC[C@@]56CCCN5C[C@H](F)C6)nc4c2F)N(CC2CCCN2)C2(CCC2)CCO3)c1]

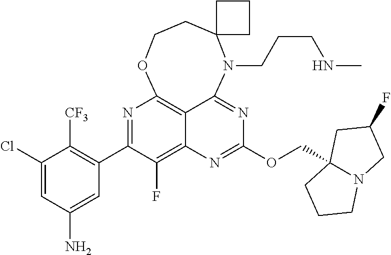 [SMILES: CNCCCN1c2nc(OC[C@@]34CCCN3C[C@H](F)C4)nc3c(F)c(-c4cc(N)cc(Cl)c4C(F)(F)F)nc(c23)OCCC12CCC2]

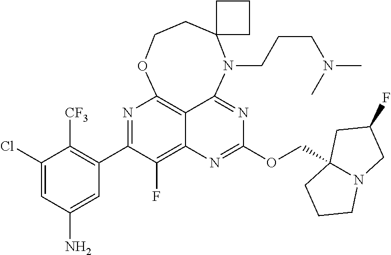 [SMILES: CN(C)CCCN1c2nc(OC[C@@]34CCCN3C[C@H](F)C4)nc3c(F)c(-c4cc(N)cc(Cl)c4C(F)(F)F)nc(c23)OCCC12CCC2]

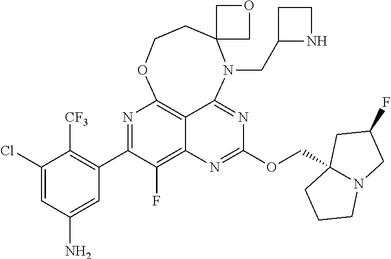 [SMILES: Nc1cc(Cl)c(C(F)(F)F)c(-c2nc3c4c(nc(OC[C@@]56CCCN5C[C@H](F)C6)nc4c2F)N(CC2CCN2)C2(CCO3)COC2)c1]

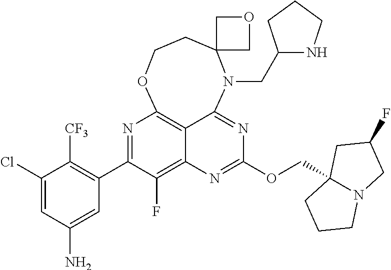 [SMILES: Nc1cc(Cl)c(C(F)(F)F)c(-c2nc3c4c(nc(OC[C@@]56CCCN5C[C@H](F)C6)nc4c2F)N(CC2CCCN2)C2(CCO3)COC2)c1]

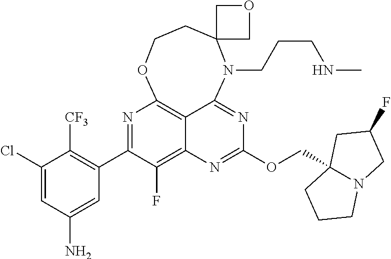 [SMILES: CNCCCN1c2nc(OC[C@@]34CCCN3C[C@H](F)C4)nc3c(F)c(-c4cc(N)cc(Cl)c4C(F)(F)F)nc(c23)OCCC12COC2]

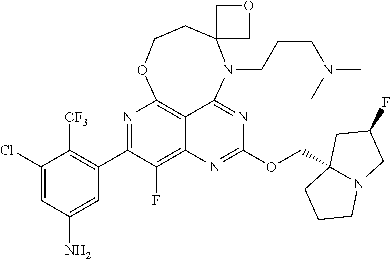 [SMILES: CN(C)CCCN1c2nc(OC[C@@]34CCCN3C[C@H](F)C4)nc3c(F)c(-c4cc(N)cc(Cl)c4C(F)(F)F)nc(c23)OCCC12COC2]

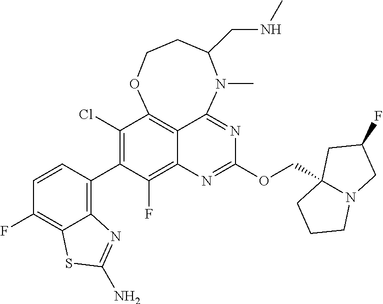 [SMILES: CNCC1CCOc2c(Cl)c(-c3ccc(F)c4sc(N)nc34)c(F)c3nc(OC[C@@]45CCCN4C[C@H](F)C5)nc(c23)N1C]

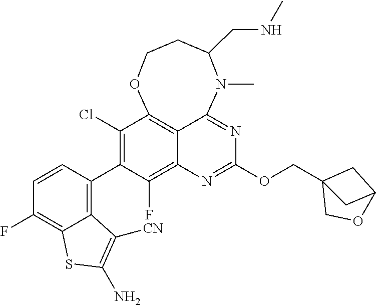 [SMILES: CNCC1CCOc2c(Cl)c(-c3ccc(F)c4sc(N)c(C#N)c34)c(F)c3nc(OCC45COC(C4)C5)nc(c23)N1C]

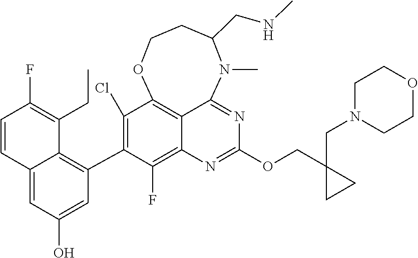 [SMILES: CCc1c(F)ccc2cc(O)cc(-c3c(Cl)c4c5c(nc(OCC6(CN7CCOCC7)CC6)nc5c3F)N(C)C(CNC)CCO4)c12]

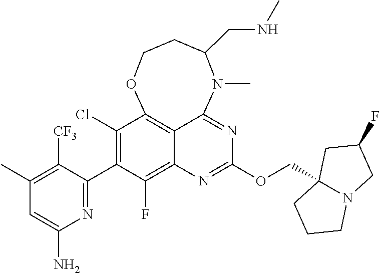 [SMILES: CNCC1CCOc2c(Cl)c(-c3nc(N)cc(C)c3C(F)(F)F)c(F)c3nc(OC[C@@]45CCCN4C[C@H](F)C5)nc(c23)N1C]

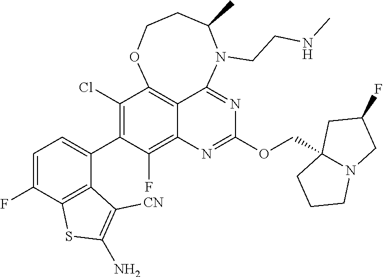 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC[C@H]1C]

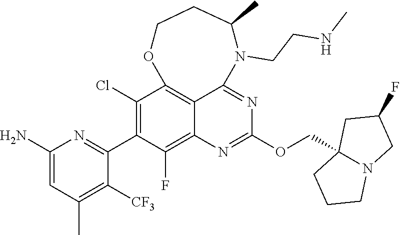 [SMILES: CNCCN1c2nc(OC[C@@]34CCCN3C[C@H](F)C4)nc3c(F)c(-c4nc(N)cc(C)c4C(F)(F)F)c(Cl)c(c23)OCC[C@H]1C]

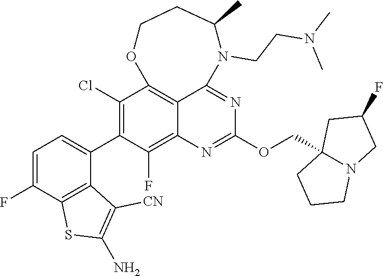 [SMILES: C[C@@H]1CCOc2c(Cl)c(-c3ccc(F)c4sc(N)c(C#N)c34)c(F)c3nc(OC[C@@]45CCCN4C[C@H](F)C5)nc(c23)N1CCN(C)C]

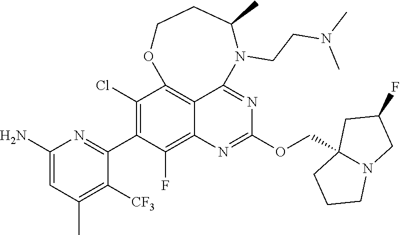 [SMILES: Cc1cc(N)nc(-c2c(Cl)c3c4c(nc(OC[C@@]56CCCN5C[C@H](F)C6)nc4c2F)N(CCN(C)C)[C@H](C)CCO3)c1C(F)(F)F]

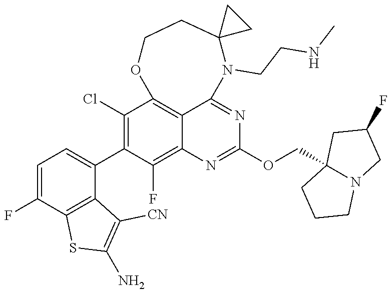 [SMILES: CNCCN1c2nc(OC[C@@]34CCCN3C[C@H](F)C4)nc3c(F)c(-c4ccc(F)c5sc(N)c(C#N)c45)c(Cl)c(c23)OCCC12CC2]

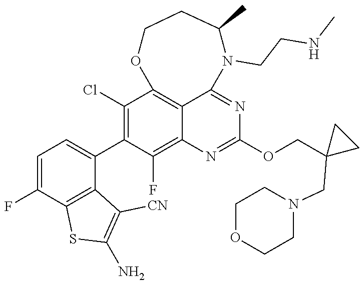 [SMILES: CNCCN1c2nc(OCC3(CN4CCOCC4)CC3)nc3c(F)c(-c4ccc(F)c5sc(N)c(C#N)c45)c(Cl)c(c23)OCC[C@H]1C]

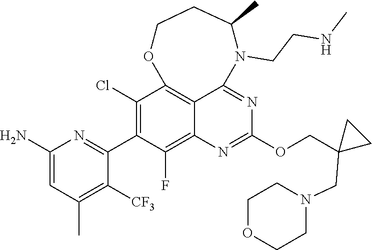 [SMILES: CNCCN1c2nc(OCC3(CN4CCOCC4)CC3)nc3c(F)c(-c4nc(N)cc(C)c4C(F)(F)F)c(Cl)c(c23)OCC[C@H]1C]

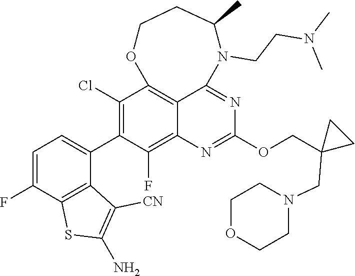 [SMILES: C[C@@H]1CCOc2c(Cl)c(-c3ccc(F)c4sc(N)c(C#N)c34)c(F)c3nc(OCC4(CN5CCOCC5)CC4)nc(c23)N1CCN(C)C]

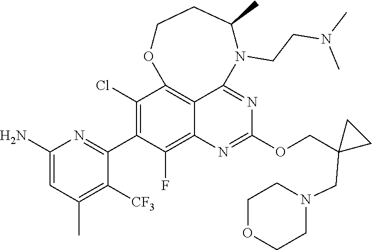 [SMILES: Cc1cc(N)nc(-c2c(Cl)c3c4c(nc(OCC5(CN6CCOCC6)CC5)nc4c2F)N(CCN(C)C)[C@H](C)CCO3)c1C(F)(F)F]

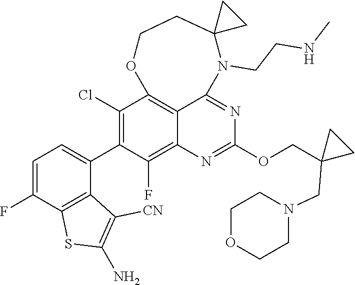 [SMILES: CNCCN1c2nc(OCC3(CN4CCOCC4)CC3)nc3c(F)c(-c4ccc(F)c5sc(N)c(C#N)c45)c(Cl)c(c23)OCCC12CC2]

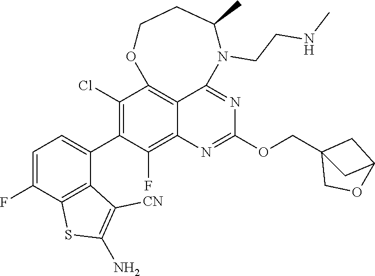 [SMILES: CNCCN1c2nc(OCC34COC(C3)C4)nc3c(F)c(-c4ccc(F)c5sc(N)c(C#N)c45)c(Cl)c(c23)OCC[C@H]1C]

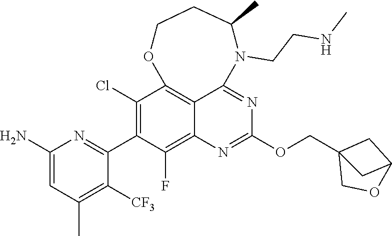 [SMILES: CNCCN1c2nc(OCC34COC(C3)C4)nc3c(F)c(-c4nc(N)cc(C)c4C(F)(F)F)c(Cl)c(c23)OCC[C@H]1C]

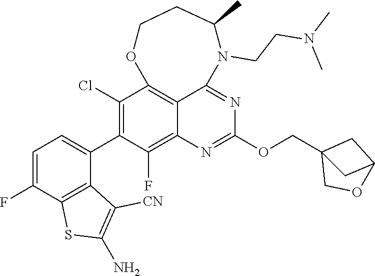 [SMILES: C[C@@H]1CCOc2c(Cl)c(-c3ccc(F)c4sc(N)c(C#N)c34)c(F)c3nc(OCC45COC(C4)C5)nc(c23)N1CCN(C)C]

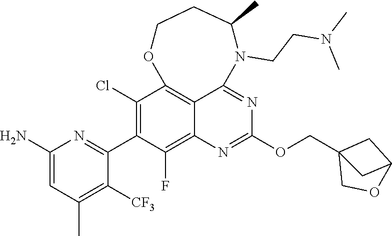 [SMILES: Cc1cc(N)nc(-c2c(Cl)c3c4c(nc(OCC56COC(C5)C6)nc4c2F)N(CCN(C)C)[C@H](C)CCO3)c1C(F)(F)F]

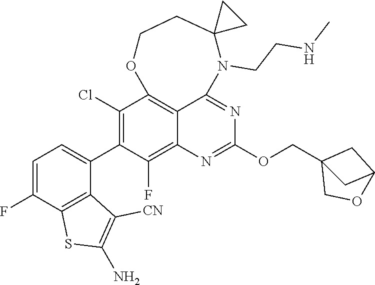 [SMILES: CNCCN1c2nc(OCC34COC(C3)C4)nc3c(F)c(-c4ccc(F)c5sc(N)c(C#N)c45)c(Cl)c(c23)OCCC12CC2]

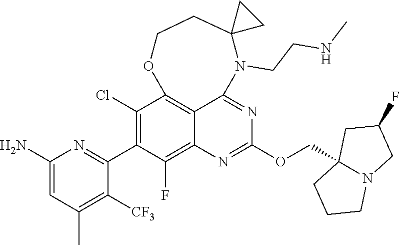 [SMILES: CNCCN1c2nc(OC[C@@]34CCCN3C[C@H](F)C4)nc3c(F)c(-c4nc(N)cc(C)c4C(F)(F)F)c(Cl)c(c23)OCCC12CC2]

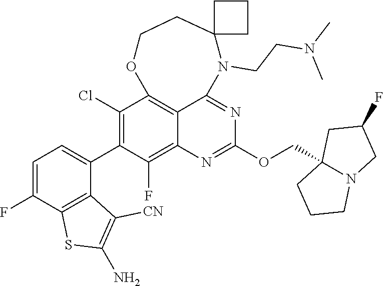 [SMILES: CN(C)CCN1c2nc(OC[C@@]34CCCN3C[C@H](F)C4)nc3c(F)c(-c4ccc(F)c5sc(N)c(C#N)c45)c(Cl)c(c23)OCCC12CCC2]

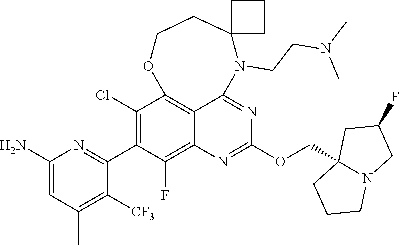 [SMILES: Cc1cc(N)nc(-c2c(Cl)c3c4c(nc(OC[C@@]56CCCN5C[C@H](F)C6)nc4c2F)N(CCN(C)C)C2(CCC2)CCO3)c1C(F)(F)F]

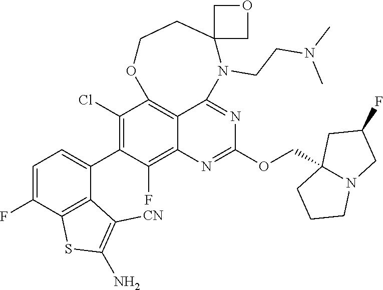 [SMILES: CN(C)CCN1c2nc(OC[C@@]34CCCN3C[C@H](F)C4)nc3c(F)c(-c4ccc(F)c5sc(N)c(C#N)c45)c(Cl)c(c23)OCCC12COC2]

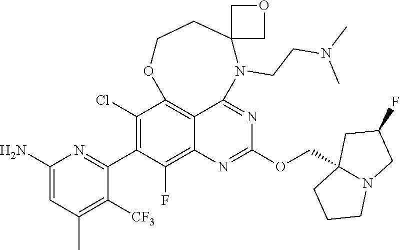 [SMILES: Cc1cc(N)nc(-c2c(Cl)c3c4c(nc(OC[C@@]56CCCN5C[C@H](F)C6)nc4c2F)N(CCN(C)C)C2(CCO3)COC2)c1C(F)(F)F]

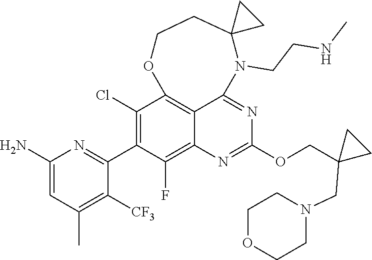 [SMILES: CNCCN1c2nc(OCC3(CN4CCOCC4)CC3)nc3c(F)c(-c4nc(N)cc(C)c4C(F)(F)F)c(Cl)c(c23)OCCC12CC2]

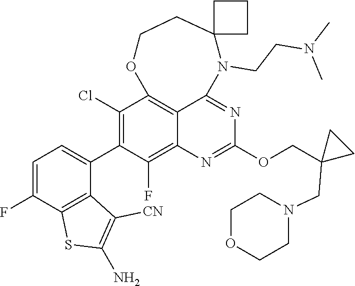 [SMILES: CN(C)CCN1c2nc(OCC3(CN4CCOCC4)CC3)nc3c(F)c(-c4ccc(F)c5sc(N)c(C#N)c45)c(Cl)c(c23)OCCC12CCC2]

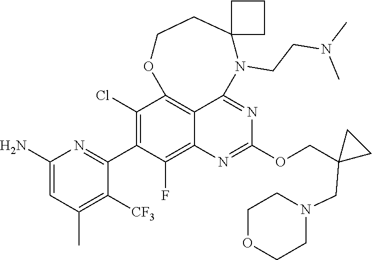 [SMILES: Cc1cc(N)nc(-c2c(Cl)c3c4c(nc(OCC5(CN6CCOCC6)CC5)nc4c2F)N(CCN(C)C)C2(CCC2)CCO3)c1C(F)(F)F]

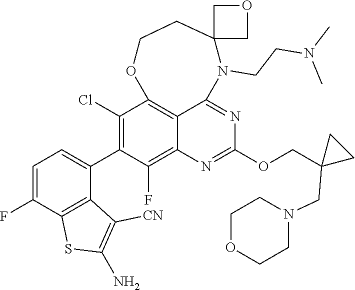 [SMILES: CN(C)CCN1c2nc(OCC3(CN4CCOCC4)CC3)nc3c(F)c(-c4ccc(F)c5sc(N)c(C#N)c45)c(Cl)c(c23)OCCC12COC2]

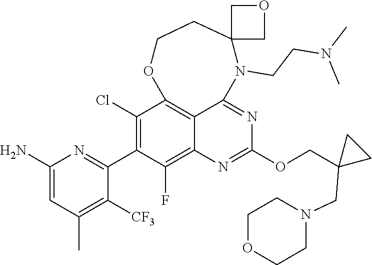 [SMILES: Cc1cc(N)nc(-c2c(Cl)c3c4c(nc(OCC5(CN6CCOCC6)CC5)nc4c2F)N(CCN(C)C)C2(CCO3)COC2)c1C(F)(F)F]

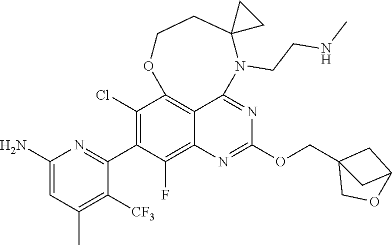 [SMILES: CNCCN1c2nc(OCC34COC(C3)C4)nc3c(F)c(-c4nc(N)cc(C)c4C(F)(F)F)c(Cl)c(c23)OCCC12CC2]

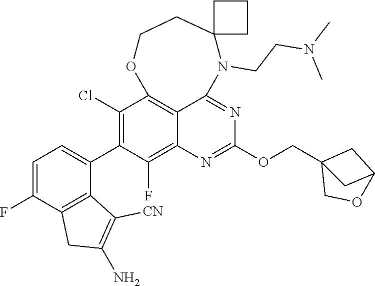 [SMILES: CN(C)CCN1c2nc(OCC34COC(C3)C4)nc3c(F)c(-c4ccc(F)c5c4C(C#N)=C(N)C5)c(Cl)c(c23)OCCC12CCC2]

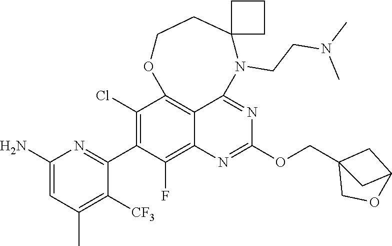 [SMILES: Cc1cc(N)nc(-c2c(Cl)c3c4c(nc(OCC56COC(C5)C6)nc4c2F)N(CCN(C)C)C2(CCC2)CCO3)c1C(F)(F)F]

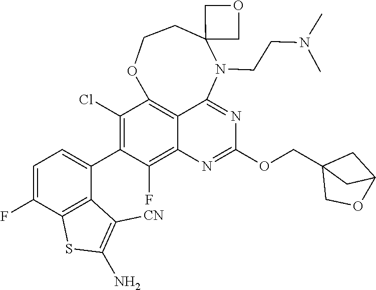 [SMILES: CN(C)CCN1c2nc(OCC34COC(C3)C4)nc3c(F)c(-c4ccc(F)c5sc(N)c(C#N)c45)c(Cl)c(c23)OCCC12COC2]

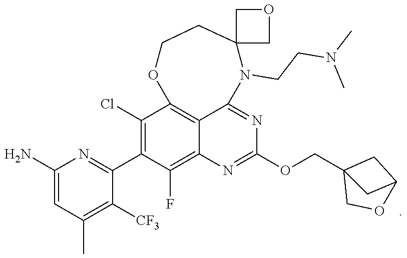 [SMILES: Cc1cc(N)nc(-c2c(Cl)c3c4c(nc(OCC56COC(C5)C6)nc4c2F)N(CCN(C)C)C2(CCO3)COC2)c1C(F)(F)F]

Aspect 14: In some embodiments, the compounds provided herein has the following formula:

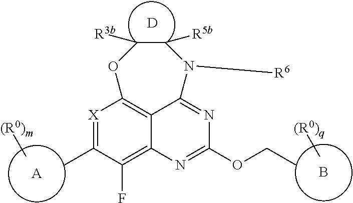

and pharmaceutically acceptable salts, tautomers, stereoisomers, enantiomers, atropisomers, isotopologues, and prodrugs thereof,
wherein:
ring D is unsubstituted or substituted 3-membered to 6-membered cycloalkyl or unsubstituted or substituted 3-membered to 6-membered heterocvclyl; or
ring D is unsubstituted or substituted 3-membered to 7-membered cycloalkyl or unsubstituted or substituted 3-membered to 7-membered heterocyclyl.

In one embodiment, ring D has one oxygen as the heteroatom.

In some embodiments, ring D is unsubstituted or substituted cyclopentyl ring, unsubstituted or substituted cyclohexyl ring, unsubstituted or substituted cycloheptyl ring, unsubstituted or substituted tetrahydrofuranyl ring, unsubstituted or substituted tetrahydropyranyl ring, or unsubstituted or substituted oxepanyl ring.

In one embodiment, ring D is optionally substituted with OH, CN, halogen, unsubstituted or substituted amino, or unsubstituted or substituted C₁₋₄ alkyl. In one embodiment, ring D is optionally substituted with OH, F, or CN.

In one embodiment, ring D is optionally substituted with 3-membered to 6-membered spirocycloalkyl or unsubstituted or substituted 3-membered to 6-membered spiroheterocyclyl. In one embodiment, ring D is optionally substituted with spirocyclopropyl.

In one embodiment, each of R^{3b}, and R^{5b}, independently, is H, OH, CN, unsubstituted or substituted amino, or unsubstituted or substituted C₁₋₄ alkyl. In one embodiment, each of R^{3b}, and R^{5b}, independently, is H, or unsubstituted or substituted methyl. In one embodiment, each of R^{3b}, and R^{5b}, independently, is H, or unsubstituted methyl. In one embodiment, each of R^{3b}, and R^{5b}, independently, is H.

In one embodiment, R⁶ is H, unsubstituted or substituted C₁₋₄ alkyl, unsubstituted or substituted C₃₋₄ cycloalkyl, or unsubstituted or substituted 3-membered to 4-membered heterocyclyl. In one embodiment, R⁶ is methyl, ethyl. 2-methylamino-ethyl, or cyclopropyl. In one embodiment, R⁶ is methyl.

In one embodiment, X is N.

In one embodiment, ring A is

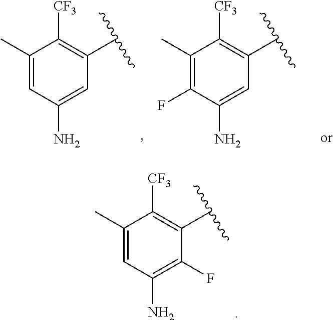

In one embodiment, ring A is

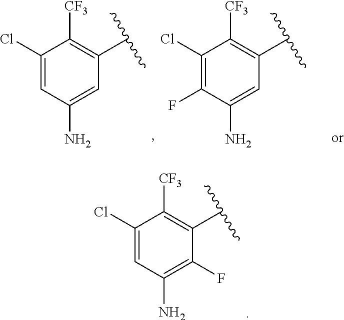

In one embodiment, ring B is

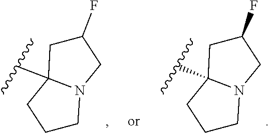 [SMILES: *C12CCCN1CC(F)C2 ; *[C@@]12CCCN1C[C@H](F)C2]

In one embodiment, ring B is

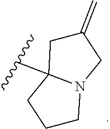 [SMILES: *C12CCCN1CC(=C)C2]

In one embodiment, ring B is

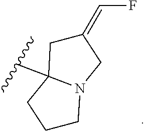 [SMILES: *C12CCCN1C/C(=C\F)C2]

In one embodiment, ring B is

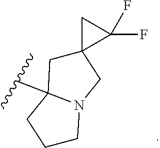 [SMILES: *C12CCCN1CC1(C2)CC1(F)F]

In one embodiment, ring B is

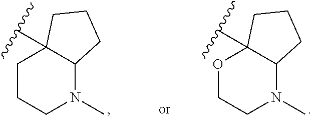 [SMILES: *C12CCCC1N(C)CCC2 ; *C12CCCC1N(C)CCO2]

In one embodiment, ring B is

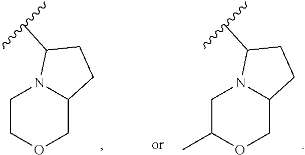 [SMILES: *C1CCC2COCCN12 ; *C1CCC2COC(C)CN12]

In one embodiment, ring B is

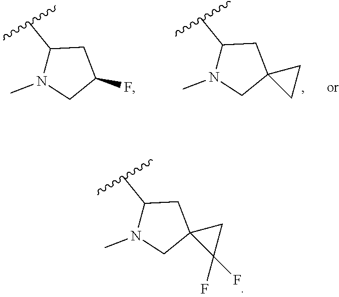

In one embodiment, ring B is

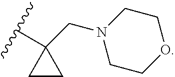 [SMILES: *C1(CN2CCOCC2)CC1]

In one embodiment, ring B is

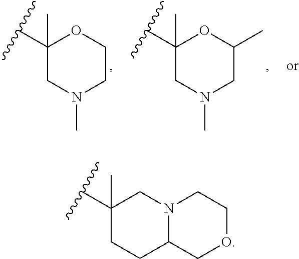

In one embodiment, ring B is

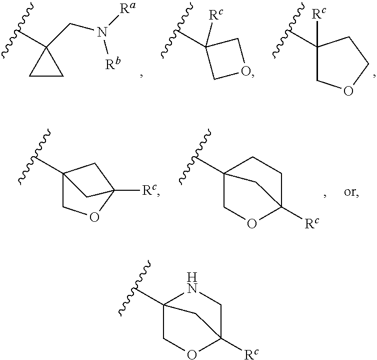

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl. In one embodiment, ring B is

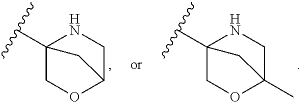

In one embodiment, the compound is

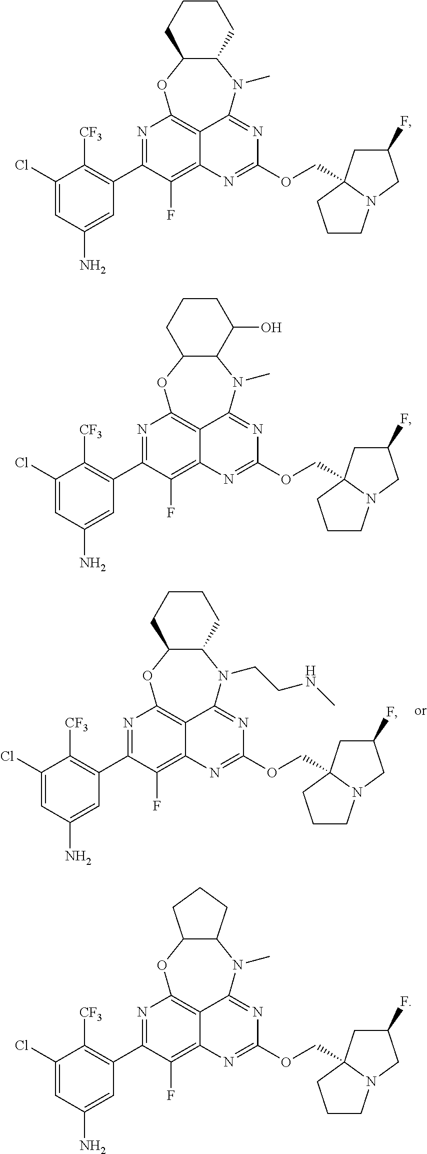 [SMILES: CN1c2nc(OC[C@@]34CCCN3C[C@H](F)C4)nc3c(F)c(-c4cc(N)cc(Cl)c4C(F)(F)F)nc(c23)O[C@H]2CCCC[C@@H]21 ; CN1c2nc(OC[C@@]34CCCN3C[C@H](F)C4)nc3c(F)c(-c4cc(N)cc(Cl)c4C(F)(F)F)nc(c23)OC2CCCC(O)C21 ; CNCCN1c2nc(OC[C@@]34CCCN3C[C@H](F)C4)nc3c(F)c(-c4cc(N)cc(Cl)c4C(F)(F)F)nc(c23)O[C@H]2CCCC[C@@H]21 ; "or" ; CN1c2nc(OC[C@@]34CCCN3C[C@H](F)C4)nc3c(F)c(-c4cc(N)cc(Cl)c4C(F)(F)F)nc(c23)OC2CCCC21]

In one embodiment, the compound is

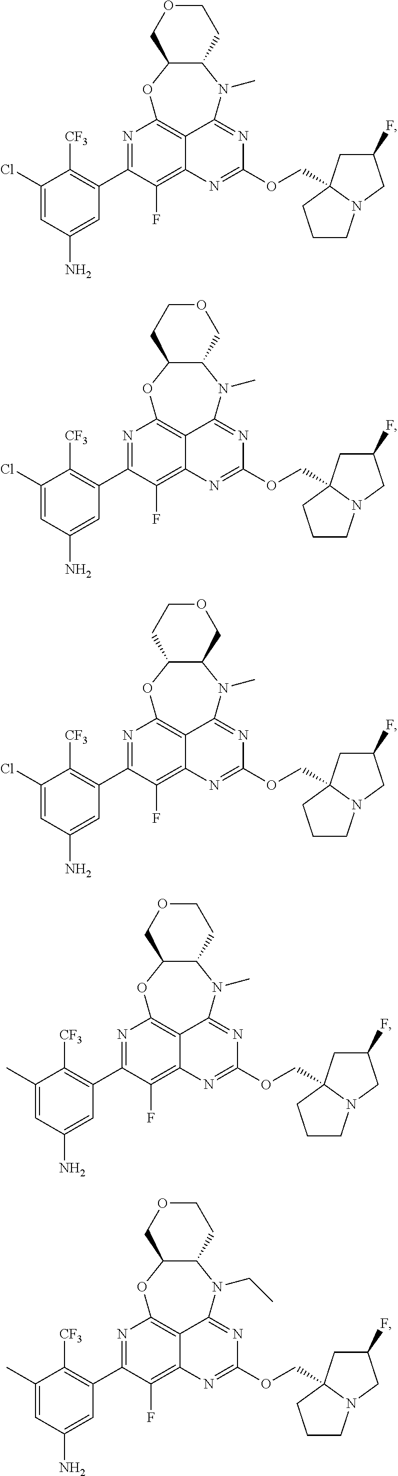 [SMILES: CN1c2nc(OC[C@@]34CCCN3C[C@H](F)C4)nc3c(F)c(-c4cc(N)cc(Cl)c4C(F)(F)F)nc(c23)O[C@H]2COCC[C@@H]21 ; CN1c2nc(OC[C@@]34CCCN3C[C@H](F)C4)nc3c(F)c(-c4cc(N)cc(Cl)c4C(F)(F)F)nc(c23)O[C@H]2CCOC[C@@H]21 ; CN1c2nc(OC[C@@]34CCCN3C[C@H](F)C4)nc3c(F)c(-c4cc(N)cc(Cl)c4C(F)(F)F)nc(c23)O[C@@H]2CCOC[C@H]21 ; Cc1cc(N)cc(-c2nc3c4c(nc(OC[C@@]56CCCN5C[C@H](F)C6)nc4c2F)N(C)[C@H]2CCOC[C@@H]2O3)c1C(F)(F)F ; CCN1c2nc(OC[C@@]34CCCN3C[C@H](F)C4)nc3c(F)c(-c4cc(N)cc(C)c4C(F)(F)F)nc(c23)O[C@H]2COCC[C@@H]21]

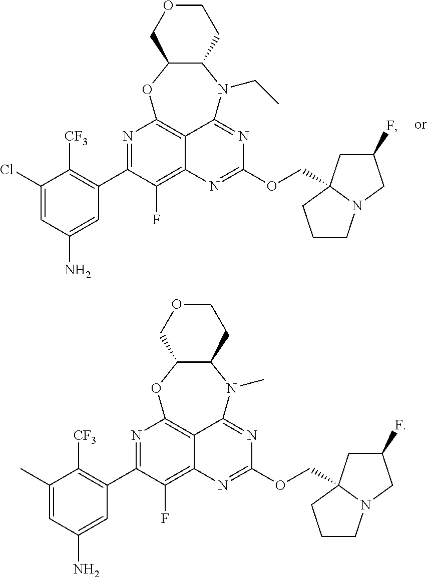 [SMILES: CCN1c2nc(OC[C@@]34CCCN3C[C@H](F)C4)nc3c(F)c(-c4cc(N)cc(Cl)c4C(F)(F)F)nc(c23)O[C@H]2COCC[C@@H]21 ; "or" ; Cc1cc(N)cc(-c2nc3c4c(nc(OC[C@@]56CCCN5C[C@H](F)C6)nc4c2F)N(C)[C@@H]2CCOC[C@H]2O3)c1C(F)(F)F]

In one embodiment, the compound is

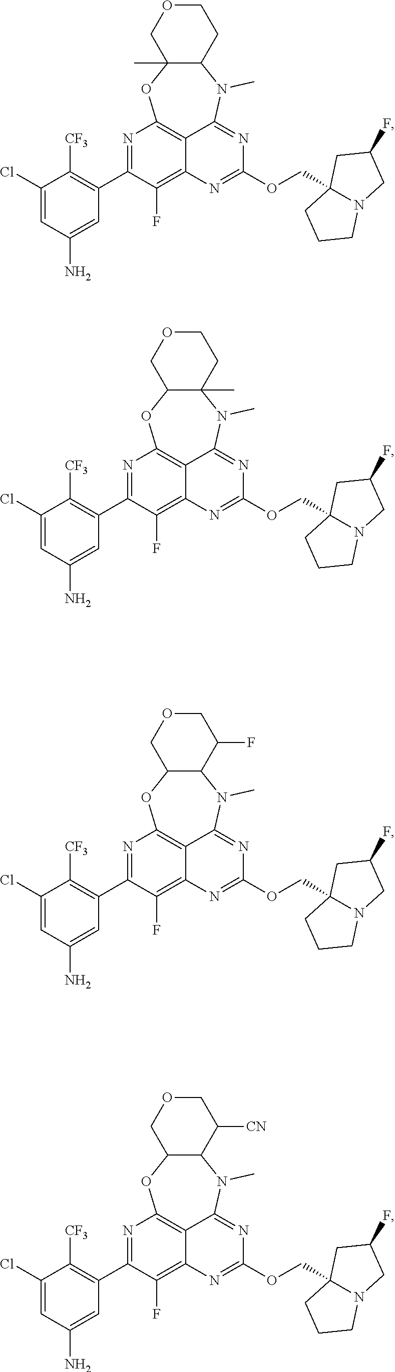 [SMILES: CN1c2nc(OC[C@@]34CCCN3C[C@H](F)C4)nc3c(F)c(-c4cc(N)cc(Cl)c4C(F)(F)F)nc(c23)OC2(C)COCCC12 ; CN1c2nc(OC[C@@]34CCCN3C[C@H](F)C4)nc3c(F)c(-c4cc(N)cc(Cl)c4C(F)(F)F)nc(c23)OC2COCCC21C ; CN1c2nc(OC[C@@]34CCCN3C[C@H](F)C4)nc3c(F)c(-c4cc(N)cc(Cl)c4C(F)(F)F)nc(c23)OC2COCC(F)C21 ; CN1c2nc(OC[C@@]34CCCN3C[C@H](F)C4)nc3c(F)c(-c4cc(N)cc(Cl)c4C(F)(F)F)nc(c23)OC2COCC(C#N)C21]

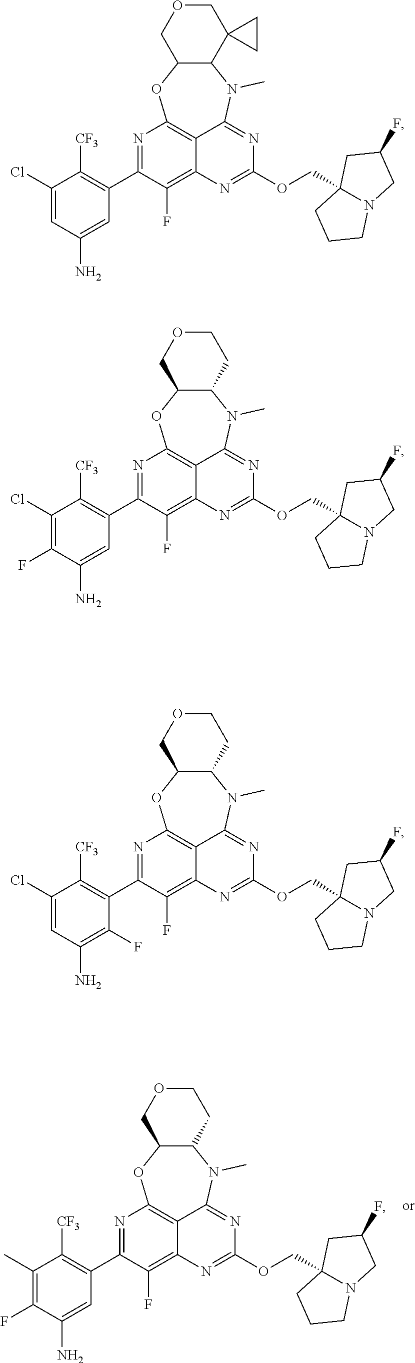

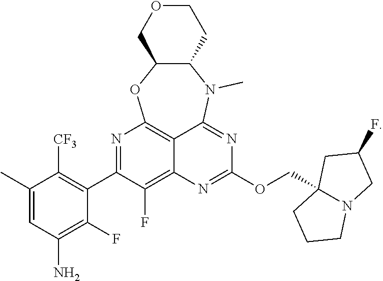 [SMILES: Cc1cc(N)c(F)c(-c2nc3c4c(nc(OC[C@@]56CCCN5C[C@H](F)C6)nc4c2F)N(C)[C@H]2CCOC[C@@H]2O3)c1C(F)(F)F]

In one embodiment, the compound is

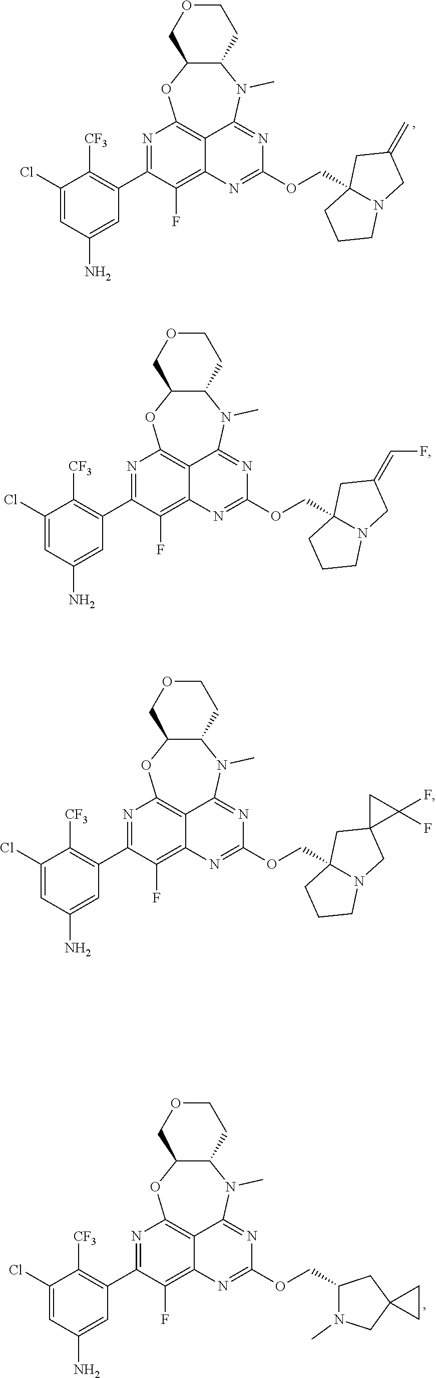 [SMILES: C=C1CN2CCC[C@@]2(COc2nc3c4c(nc(-c5cc(N)cc(Cl)c5C(F)(F)F)c(F)c4n2)O[C@H]2COCC[C@@H]2N3C)C1 ; CN1c2nc(OC[C@@]34CCCN3C/C(=C\F)C4)nc3c(F)c(-c4cc(N)cc(Cl)c4C(F)(F)F)nc(c23)O[C@H]2COCC[C@@H]21 ; CN1c2nc(OC[C@@]34CCCN3CC3(CC3(F)F)C4)nc3c(F)c(-c4cc(N)cc(Cl)c4C(F)(F)F)nc(c23)O[C@H]2COCC[C@@H]21 ; CN1CC2(CC2)C[C@H]1COc1nc2c3c(nc(-c4cc(N)cc(Cl)c4C(F)(F)F)c(F)c3n1)O[C@H]1COCC[C@@H]1N2C]

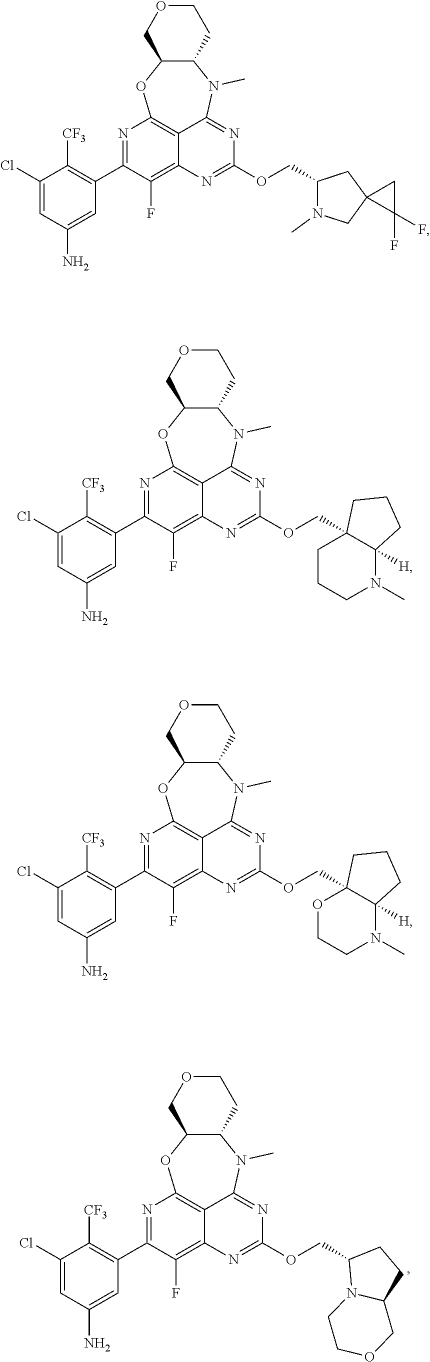

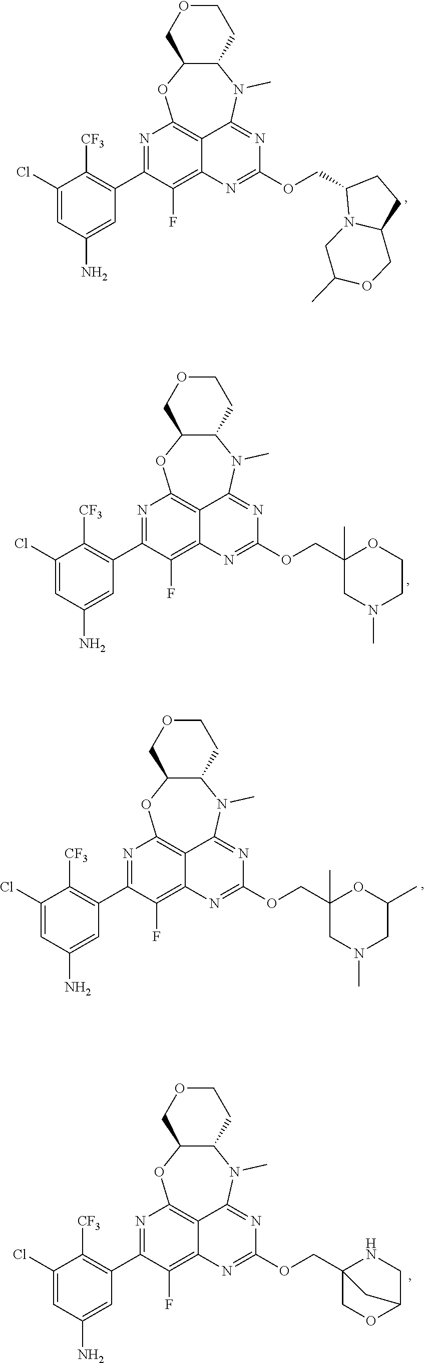 [SMILES: CC1CN2[C@H](COc3nc4c5c(nc(-c6cc(N)cc(Cl)c6C(F)(F)F)c(F)c5n3)O[C@H]3COCC[C@@H]3N4C)CC[C@H]2CO1 ; CN1CCOC(C)(COc2nc3c4c(nc(-c5cc(N)cc(Cl)c5C(F)(F)F)c(F)c4n2)O[C@H]2COCC[C@@H]2N3C)C1 ; CC1CN(C)CC(C)(COc2nc3c4c(nc(-c5cc(N)cc(Cl)c5C(F)(F)F)c(F)c4n2)O[C@H]2COCC[C@@H]2N3C)O1 ; CN1c2nc(OCC34COC(CN3)C4)nc3c(F)c(-c4cc(N)cc(Cl)c4C(F)(F)F)nc(c23)O[C@H]2COCC[C@@H]21]

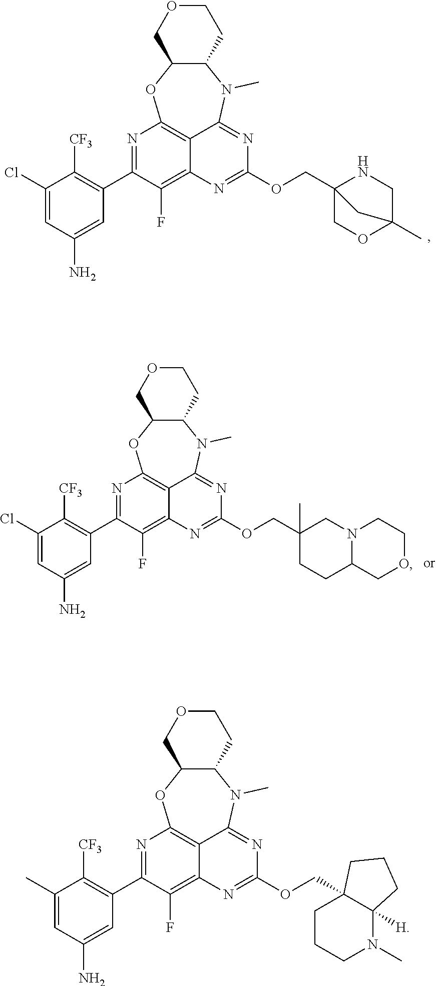

In one embodiment, the compound is

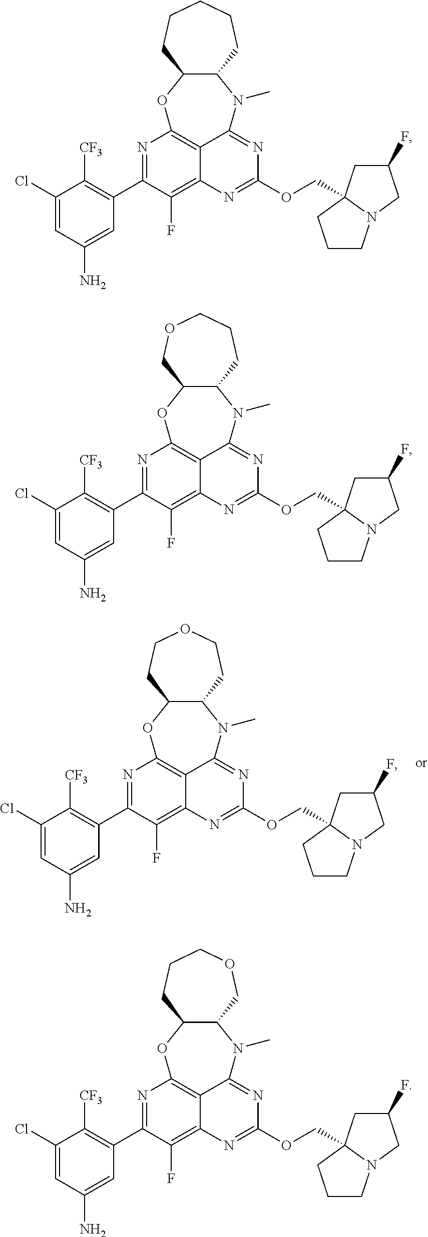 [SMILES: CN1c2nc(OC[C@@]34CCCN3C[C@H](F)C4)nc3c(F)c(-c4cc(N)cc(Cl)c4C(F)(F)F)nc(c23)O[C@H]2CCCCC[C@@H]21 ; CN1c2nc(OC[C@@]34CCCN3C[C@H](F)C4)nc3c(F)c(-c4cc(N)cc(Cl)c4C(F)(F)F)nc(c23)O[C@H]2COCCC[C@@H]21 ; CN1c2nc(OC[C@@]34CCCN3C[C@H](F)C4)nc3c(F)c(-c4cc(N)cc(Cl)c4C(F)(F)F)nc(c23)O[C@H]2CCOCC[C@@H]21 ; "or" ; CN1c2nc(OC[C@@]34CCCN3C[C@H](F)C4)nc3c(F)c(-c4cc(N)cc(Cl)c4C(F)(F)F)nc(c23)O[C@H]2CCCOC[C@@H]21]

Aspect 15: In some embodiments, the compounds provided herein has one of the following formulas:

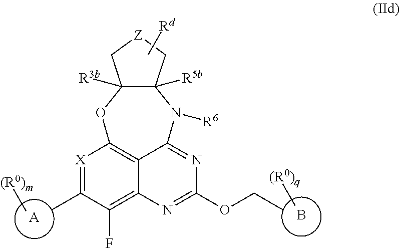

and pharmaceutically acceptable salts, tautomers, stereoisomers, enantiomers, atropisomers, isotopologues, and prodrugs thereof,
wherein:
Z is CH₂, NH, or O; and
R^{d} is H, halogen, amino, —CN, —OH, unsubstituted or substituted C₁₋₄ alkyl, unsubstituted or substituted C₁₋₄ alkoxy, or unsubstituted or substituted C₁₋₄ alkylamino.

In one embodiment, Z is CH₂. In one embodiment, Z is O. In one embodiment, Z is NH.

Aspect 16: Provided herein is a compound selected from the following table:

TABLE 2

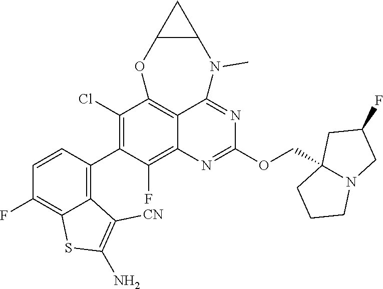 [SMILES: CN1c2nc(OC[C@@]34CCCN3C[C@H](F)C4)nc3c(F)c(-c4ccc(F)c5sc(N)c(C#N)c45)c(Cl)c(c23)OC2CC21]

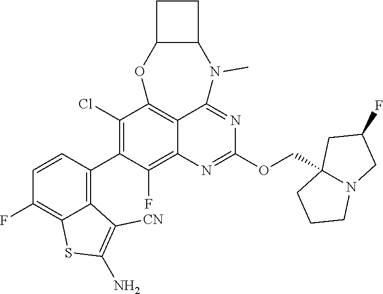 [SMILES: CN1c2nc(OC[C@@]34CCCN3C[C@H](F)C4)nc3c(F)c(-c4ccc(F)c5sc(N)c(C#N)c45)c(Cl)c(c23)OC2CCC21]

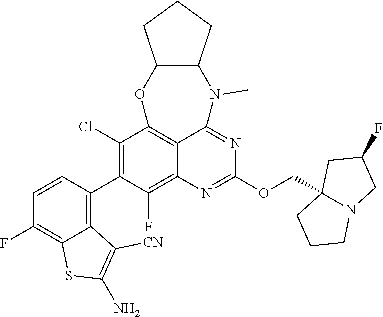 [SMILES: CN1c2nc(OC[C@@]34CCCN3C[C@H](F)C4)nc3c(F)c(-c4ccc(F)c5sc(N)c(C#N)c45)c(Cl)c(c23)OC2CCCC21]

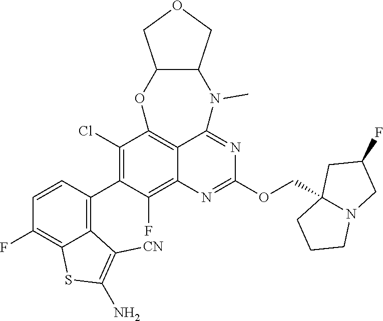 [SMILES: CN1c2nc(OC[C@@]34CCCN3C[C@H](F)C4)nc3c(F)c(-c4ccc(F)c5sc(N)c(C#N)c45)c(Cl)c(c23)OC2COCC21]

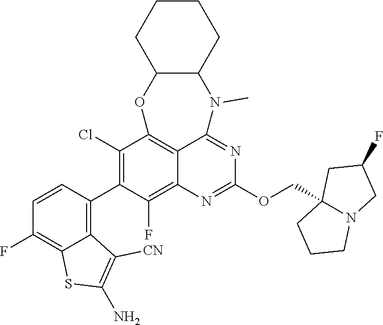 [SMILES: CN1c2nc(OC[C@@]34CCCN3C[C@H](F)C4)nc3c(F)c(-c4ccc(F)c5sc(N)c(C#N)c45)c(Cl)c(c23)OC2CCCCC21]

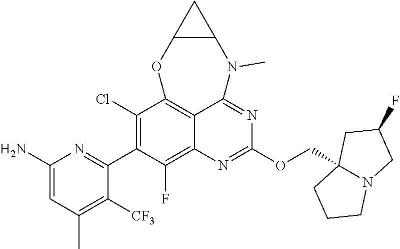 [SMILES: Cc1cc(N)nc(-c2c(Cl)c3c4c(nc(OC[C@@]56CCCN5C[C@H](F)C6)nc4c2F)N(C)C2CC2O3)c1C(F)(F)F]

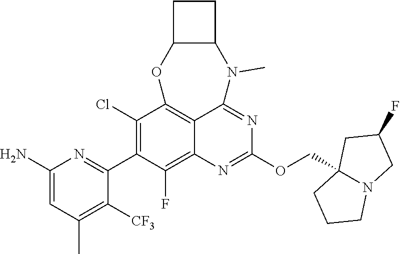 [SMILES: Cc1cc(N)nc(-c2c(Cl)c3c4c(nc(OC[C@@]56CCCN5C[C@H](F)C6)nc4c2F)N(C)C2CCC2O3)c1C(F)(F)F]

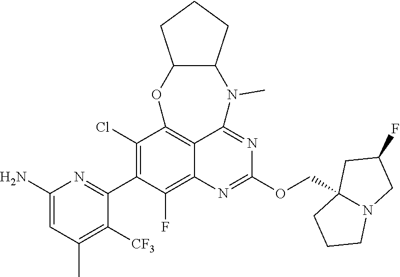 [SMILES: Cc1cc(N)nc(-c2c(Cl)c3c4c(nc(OC[C@@]56CCCN5C[C@H](F)C6)nc4c2F)N(C)C2CCCC2O3)c1C(F)(F)F]

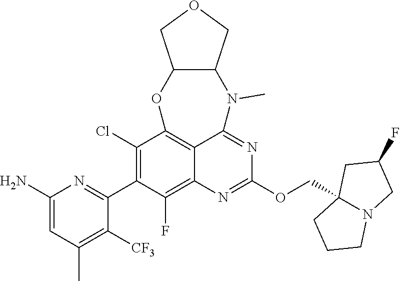 [SMILES: Cc1cc(N)nc(-c2c(Cl)c3c4c(nc(OC[C@@]56CCCN5C[C@H](F)C6)nc4c2F)N(C)C2COCC2O3)c1C(F)(F)F]

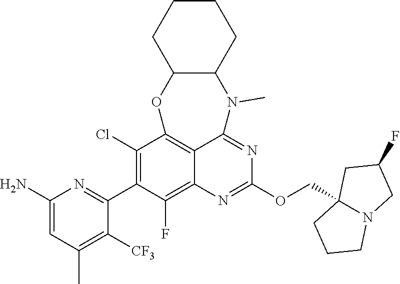 [SMILES: Cc1cc(N)nc(-c2c(Cl)c3c4c(nc(OC[C@@]56CCCN5C[C@H](F)C6)nc4c2F)N(C)C2CCCCC2O3)c1C(F)(F)F]

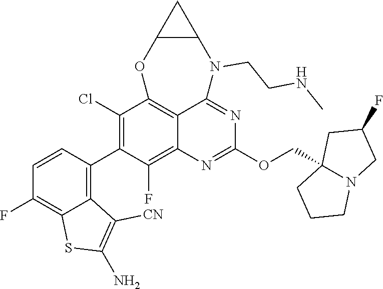 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CC21]

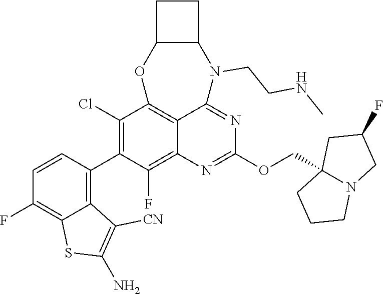 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CCC21]

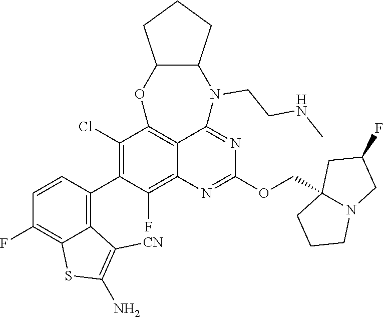 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CCCC21]

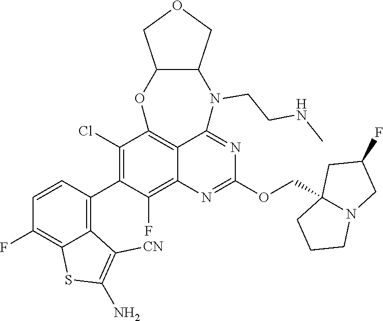 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2COCC21]

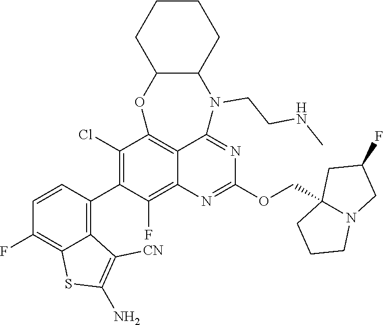 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CCCCC21]

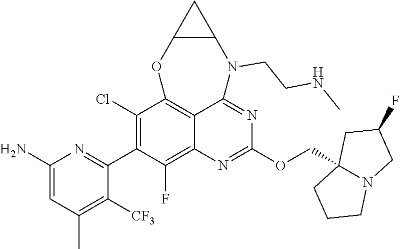 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CC21]

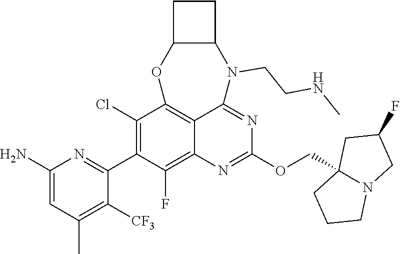 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CCC21]

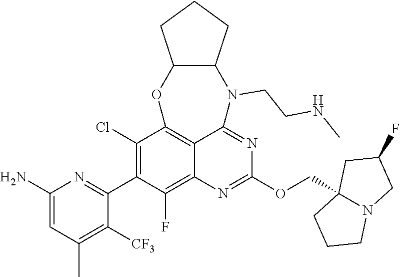 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CCCC21]

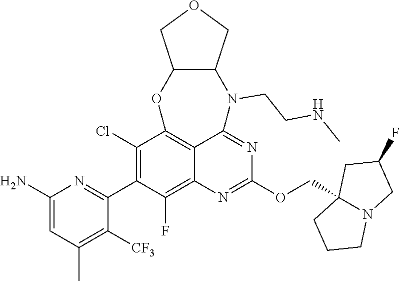 [SMILES: CNCCN1c2nc(OC[C@@]34CCCN3C[C@H](F)C4)nc3c(F)c(-c4nc(N)cc(C)c4C(F)(F)F)c(Cl)c(c23)OC2COCC21]

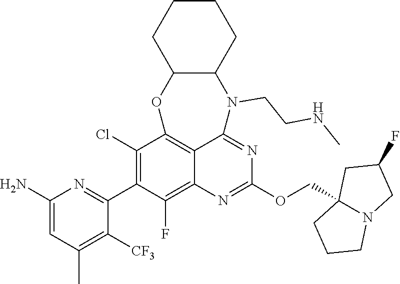 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CCCCC21]

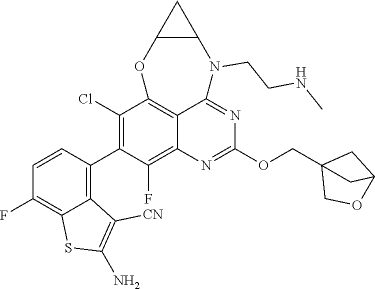 [SMILES: CNCCN1c2nc(OCC34COC(C3)C4)nc3c(F)c(-c4ccc(F)c5sc(N)c(C#N)c45)c(Cl)c(c23)OC2CC21]

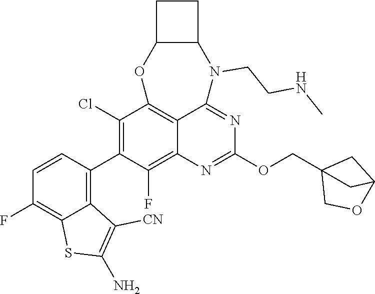 [SMILES: CNCCN1c2nc(OCC34COC(C3)C4)nc3c(F)c(-c4ccc(F)c5sc(N)c(C#N)c45)c(Cl)c(c23)OC2CCC21]

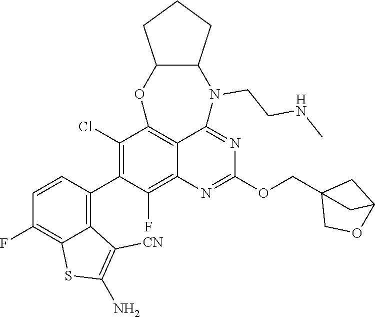 [SMILES: CNCCN1c2nc(OCC34COC(C3)C4)nc3c(F)c(-c4ccc(F)c5sc(N)c(C#N)c45)c(Cl)c(c23)OC2CCCC21]

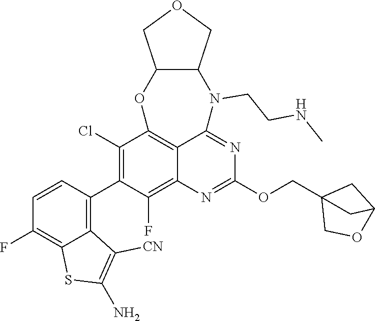 [SMILES: CNCCN1c2nc(OCC34COC(C3)C4)nc3c(F)c(-c4ccc(F)c5sc(N)c(C#N)c45)c(Cl)c(c23)OC2COCC21]

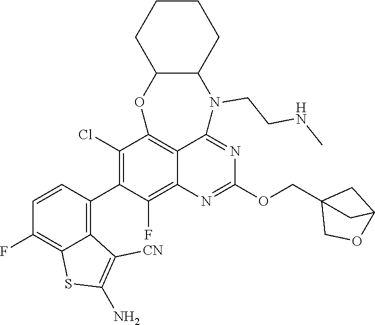 [SMILES: CNCCN1c2nc(OCC34COC(C3)C4)nc3c(F)c(-c4ccc(F)c5sc(N)c(C#N)c45)c(Cl)c(c23)OC2CCCCC21]

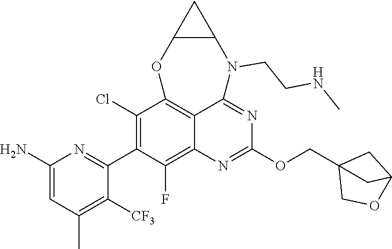 [SMILES: CNCCN1c2nc(OCC34COC(C3)C4)nc3c(F)c(-c4nc(N)cc(C)c4C(F)(F)F)c(Cl)c(c23)OC2CC21]

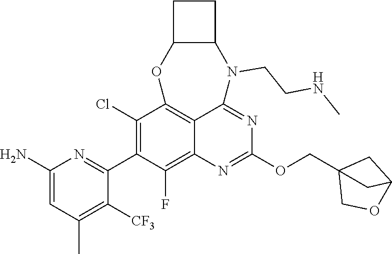 [SMILES: CNCCN1c2nc(OCC34COC(C3)C4)nc3c(F)c(-c4nc(N)cc(C)c4C(F)(F)F)c(Cl)c(c23)OC2CCC21]

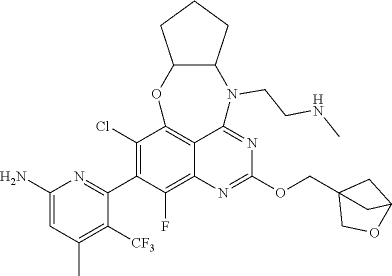 [SMILES: CNCCN1c2nc(OCC34COC(C3)C4)nc3c(F)c(-c4nc(N)cc(C)c4C(F)(F)F)c(Cl)c(c23)OC2CCCC21]

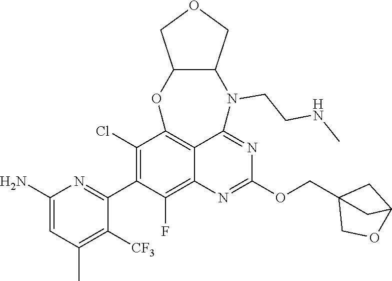 [SMILES: CNCCN1c2nc(OCC34COC(C3)C4)nc3c(F)c(-c4nc(N)cc(C)c4C(F)(F)F)c(Cl)c(c23)OC2COCC21]

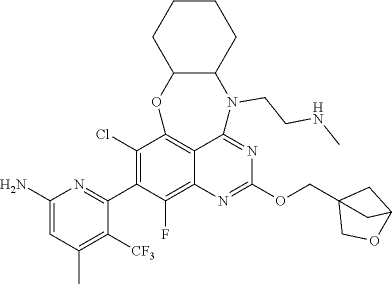 [SMILES: CNCCN1c2nc(OCC34COC(C3)C4)nc3c(F)c(-c4nc(N)cc(C)c4C(F)(F)F)c(Cl)c(c23)OC2CCCCC21]

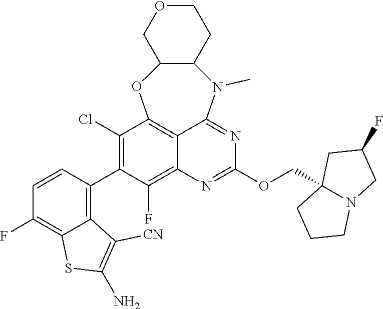 [SMILES: CN1c2nc(OC[C@@]34CCCN3C[C@H](F)C4)nc3c(F)c(-c4ccc(F)c5sc(N)c(C#N)c45)c(Cl)c(c23)OC2COCCC21]

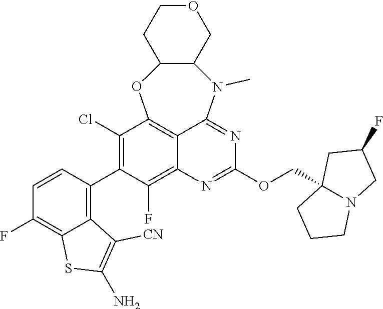 [SMILES: CN1c2nc(OC[C@@]34CCCN3C[C@H](F)C4)nc3c(F)c(-c4ccc(F)c5sc(N)c(C#N)c45)c(Cl)c(c23)OC2CCOCC21]

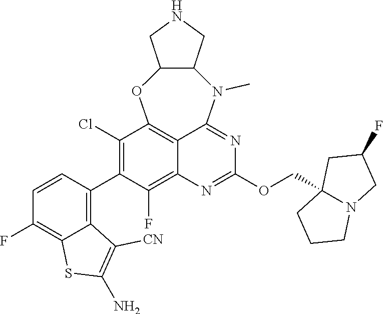 [SMILES: CN1c2nc(OC[C@@]34CCCN3C[C@H](F)C4)nc3c(F)c(-c4ccc(F)c5sc(N)c(C#N)c45)c(Cl)c(c23)OC2CNCC21]

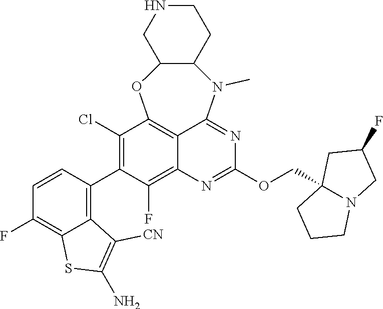 [SMILES: CN1c2nc(OC[C@@]34CCCN3C[C@H](F)C4)nc3c(F)c(-c4ccc(F)c5sc(N)c(C#N)c45)c(Cl)c(c23)OC2CNCCC21]

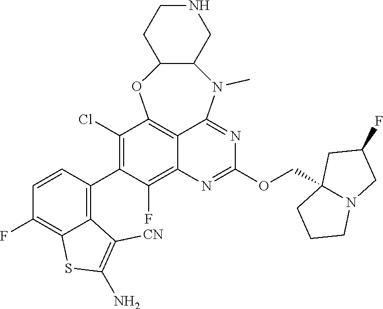 [SMILES: CN1c2nc(OC[C@@]34CCCN3C[C@H](F)C4)nc3c(F)c(-c4ccc(F)c5sc(N)c(C#N)c45)c(Cl)c(c23)OC2CCNCC21]

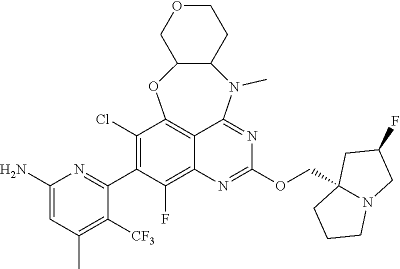 [SMILES: Cc1cc(N)nc(-c2c(Cl)c3c4c(nc(OC[C@@]56CCCN5C[C@H](F)C6)nc4c2F)N(C)C2CCOCC2O3)c1C(F)(F)F]

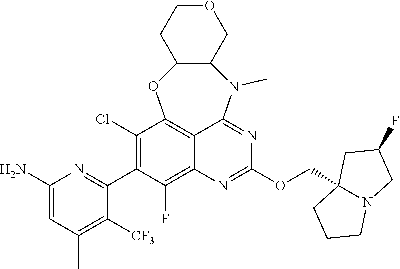 [SMILES: Cc1cc(N)nc(-c2c(Cl)c3c4c(nc(OC[C@@]56CCCN5C[C@H](F)C6)nc4c2F)N(C)C2COCCC2O3)c1C(F)(F)F]

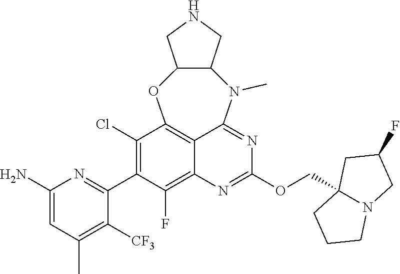 [SMILES: Cc1cc(N)nc(-c2c(Cl)c3c4c(nc(OC[C@@]56CCCN5C[C@H](F)C6)nc4c2F)N(C)C2CNCC2O3)c1C(F)(F)F]

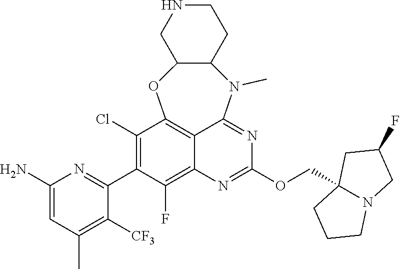 [SMILES: Cc1cc(N)nc(-c2c(Cl)c3c4c(nc(OC[C@@]56CCCN5C[C@H](F)C6)nc4c2F)N(C)C2CCNCC2O3)c1C(F)(F)F]

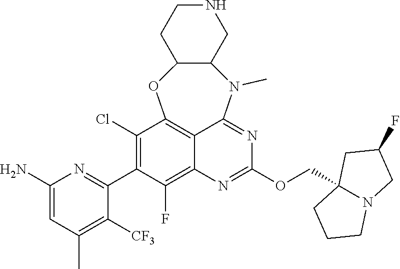 [SMILES: Cc1cc(N)nc(-c2c(Cl)c3c4c(nc(OC[C@@]56CCCN5C[C@H](F)C6)nc4c2F)N(C)C2CNCCC2O3)c1C(F)(F)F]

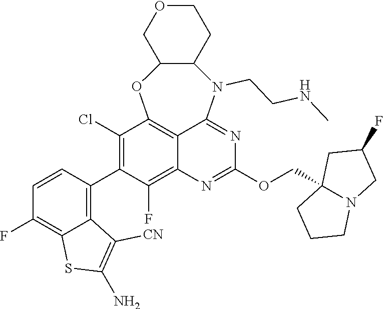 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2COCCC21]

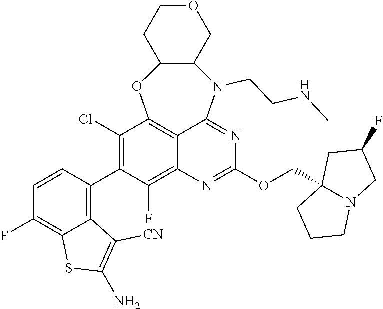 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CCOCC21]

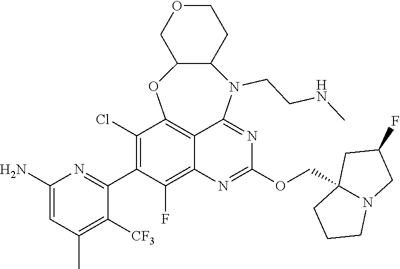 [SMILES: CNCCN1c2nc(OC[C@@]34CCCN3C[C@H](F)C4)nc3c(F)c(-c4nc(N)cc(C)c4C(F)(F)F)c(Cl)c(c23)OC2COCCC21]

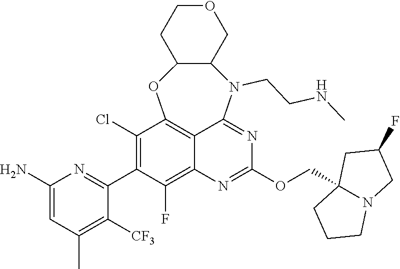 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CCOCC21]

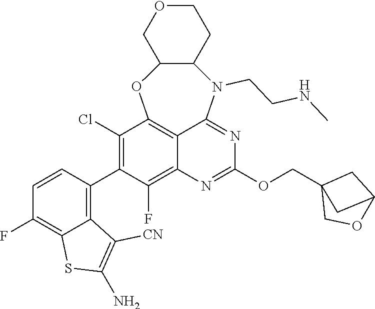 [SMILES: CNCCN1c2nc(OCC34COC(C3)C4)nc3c(F)c(-c4ccc(F)c5sc(N)c(C#N)c45)c(Cl)c(c23)OC2COCCC21]

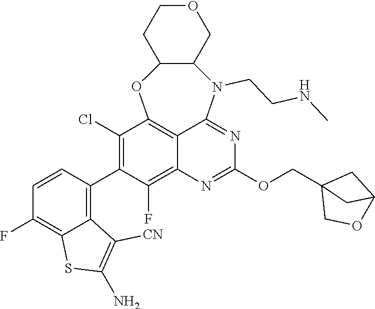 [SMILES: CNCCN1c2nc(OCC34COC(C3)C4)nc3c(F)c(-c4ccc(F)c5sc(N)c(C#N)c45)c(Cl)c(c23)OC2CCOCC21]

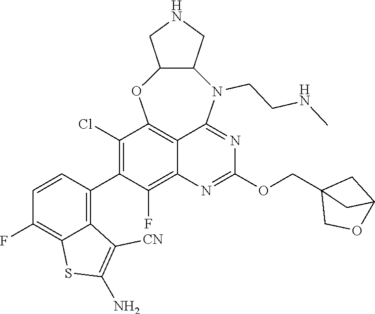 [SMILES: CNCCN1c2nc(OCC34COC(C3)C4)nc3c(F)c(-c4ccc(F)c5sc(N)c(C#N)c45)c(Cl)c(c23)OC2CNCC21]

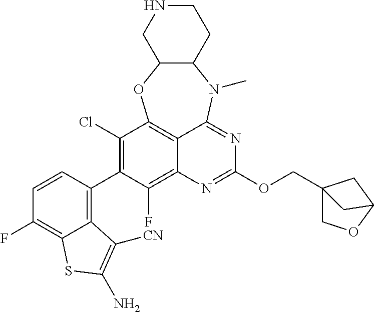 [SMILES: CN1c2nc(OCC34COC(C3)C4)nc3c(F)c(-c4ccc(F)c5sc(N)c(C#N)c45)c(Cl)c(c23)OC2CNCCC21]

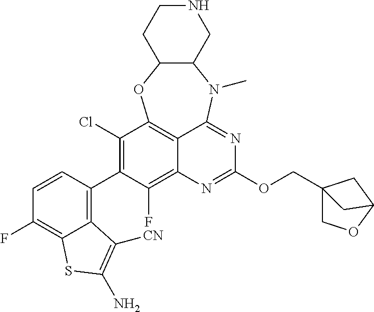 [SMILES: CN1c2nc(OCC34COC(C3)C4)nc3c(F)c(-c4ccc(F)c5sc(N)c(C#N)c45)c(Cl)c(c23)OC2CCNCC21]

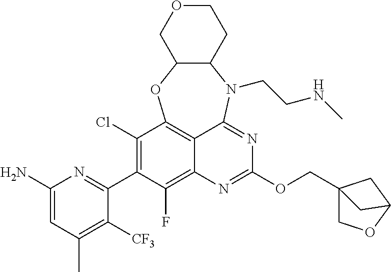 [SMILES: CNCCN1c2nc(OCC34COC(C3)C4)nc3c(F)c(-c4nc(N)cc(C)c4C(F)(F)F)c(Cl)c(c23)OC2COCCC21]

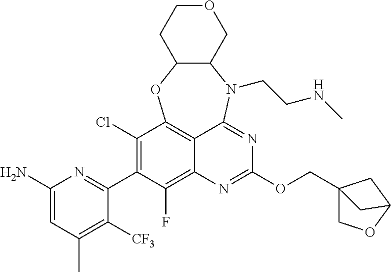 [SMILES: CNCCN1c2nc(OCC34COC(C3)C4)nc3c(F)c(-c4nc(N)cc(C)c4C(F)(F)F)c(Cl)c(c23)OC2CCOCC21]

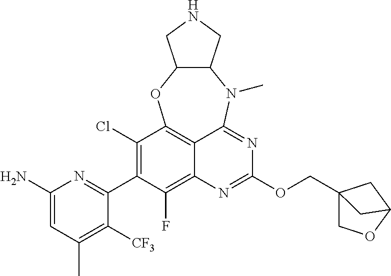 [SMILES: Cc1cc(N)nc(-c2c(Cl)c3c4c(nc(OCC56COC(C5)C6)nc4c2F)N(C)C2CNCC2O3)c1C(F)(F)F]

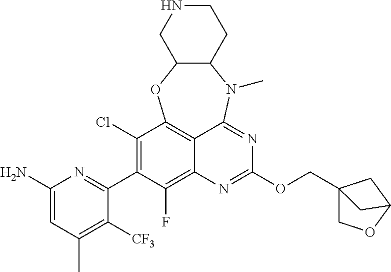 [SMILES: Cc1cc(N)nc(-c2c(Cl)c3c4c(nc(OCC56COC(C5)C6)nc4c2F)N(C)C2CCNCC2O3)c1C(F)(F)F]

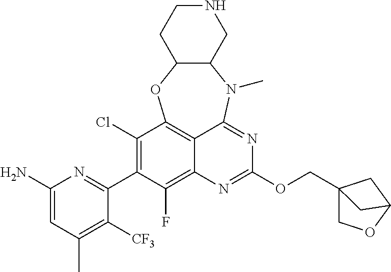 [SMILES: Cc1cc(N)nc(-c2c(Cl)c3c4c(nc(OCC56COC(C5)C6)nc4c2F)N(C)C2CNCCC2O3)c1C(F)(F)F]

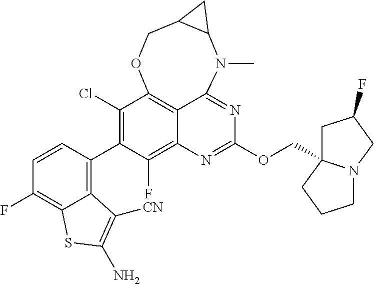 [SMILES: CN1c2nc(OC[C@@]34CCCN3C[C@H](F)C4)nc3c(F)c(-c4ccc(F)c5sc(N)c(C#N)c45)c(Cl)c(c23)OCC2CC21]

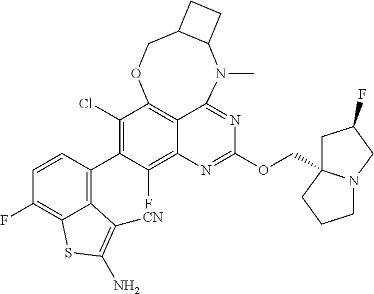 [SMILES: CN1c2nc(OC[C@@]34CCCN3C[C@H](F)C4)nc3c(F)c(-c4ccc(F)c5sc(N)c(C#N)c45)c(Cl)c(c23)OCC2CCC21]

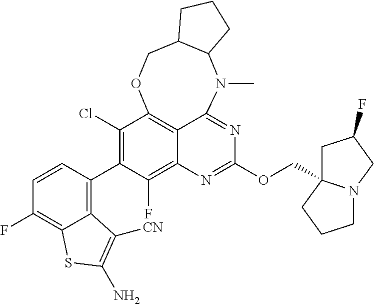 [SMILES: CN1c2nc(OC[C@@]34CCCN3C[C@H](F)C4)nc3c(F)c(-c4ccc(F)c5sc(N)c(C#N)c45)c(Cl)c(c23)OCC2CCCC21]

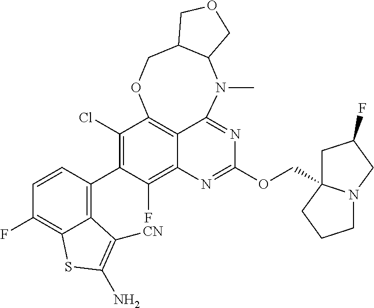 [SMILES: CN1c2nc(OC[C@@]34CCCN3C[C@H](F)C4)nc3c(F)c(-c4ccc(F)c5sc(N)c(C#N)c45)c(Cl)c(c23)OCC2COCC21]

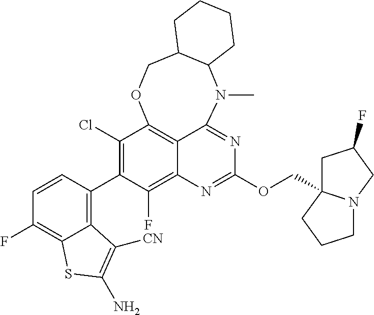 [SMILES: CN1c2nc(OC[C@@]34CCCN3C[C@H](F)C4)nc3c(F)c(-c4ccc(F)c5sc(N)c(C#N)c45)c(Cl)c(c23)OCC2CCCCC21]

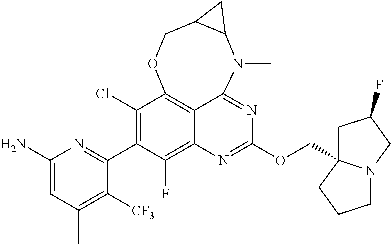 [SMILES: Cc1cc(N)nc(-c2c(Cl)c3c4c(nc(OC[C@@]56CCCN5C[C@H](F)C6)nc4c2F)N(C)C2CC2CO3)c1C(F)(F)F]

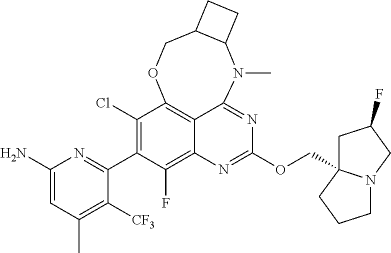 [SMILES: Cc1cc(N)nc(-c2c(Cl)c3c4c(nc(OC[C@@]56CCCN5C[C@H](F)C6)nc4c2F)N(C)C2CCC2CO3)c1C(F)(F)F]

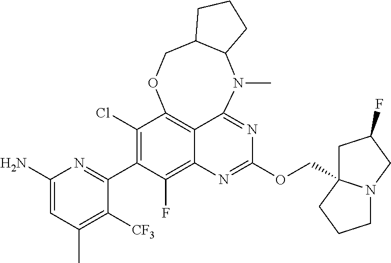 [SMILES: Cc1cc(N)nc(-c2c(Cl)c3c4c(nc(OC[C@@]56CCCN5C[C@H](F)C6)nc4c2F)N(C)C2CCCC2CO3)c1C(F)(F)F]

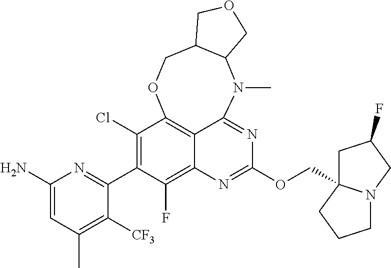 [SMILES: Cc1cc(N)nc(-c2c(Cl)c3c4c(nc(OC[C@@]56CCCN5C[C@H](F)C6)nc4c2F)N(C)C2COCC2CO3)c1C(F)(F)F]

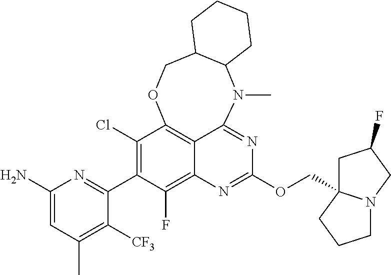 [SMILES: Cc1cc(N)nc(-c2c(Cl)c3c4c(nc(OC[C@@]56CCCN5C[C@H](F)C6)nc4c2F)N(C)C2CCCCC2CO3)c1C(F)(F)F]

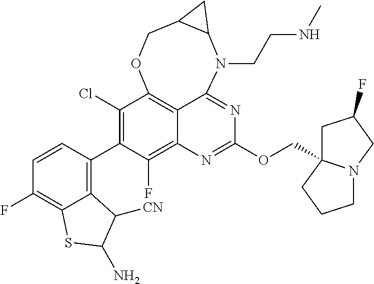 [SMILES: CNCCN1c2nc(OC[C@@]34CCCN3C[C@H](F)C4)nc3c(F)c(-c4ccc(F)c5c4C(C#N)C(N)S5)c(Cl)c(c23)OCC2CC21]

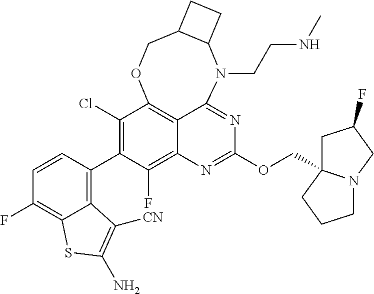 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC2CCC21]

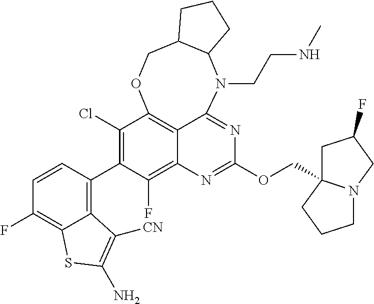 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC2CCCC21]

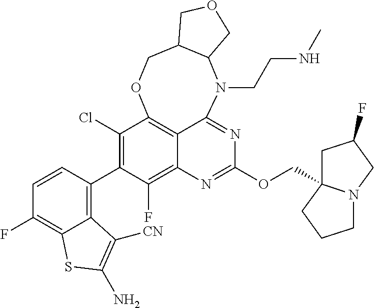 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC2COCC21]

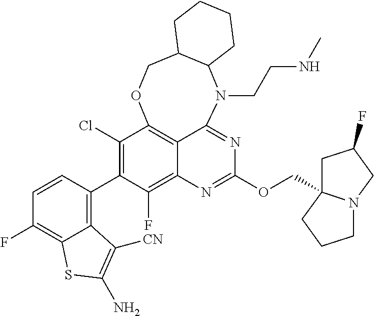 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC2CCCCC21]

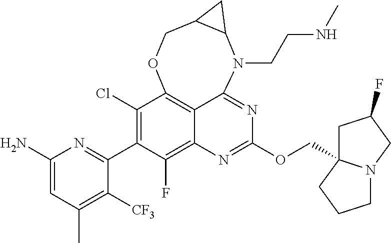 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2CC21]

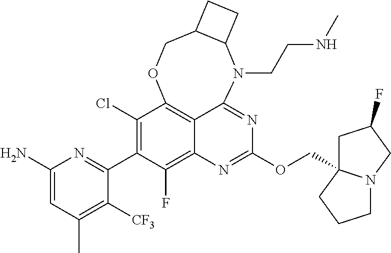 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2CCC21]

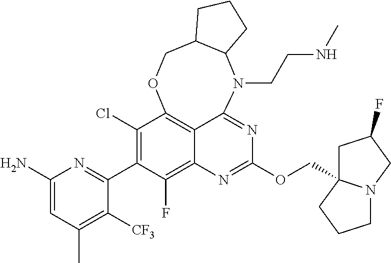 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2CCCC21]

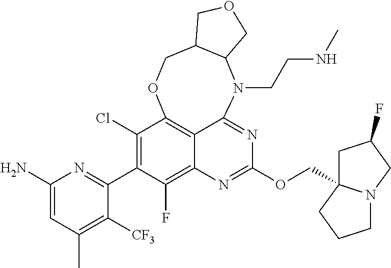 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2COCC21]

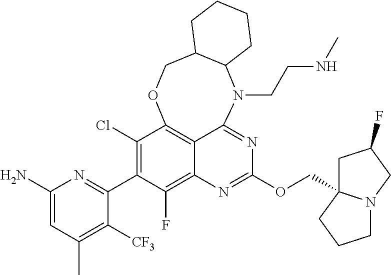 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2CCCCC21]

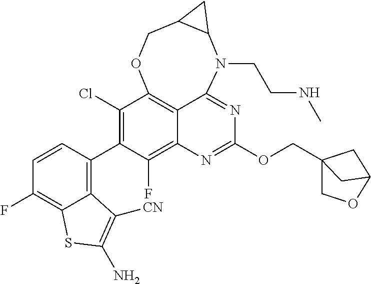 [SMILES: CNCCN1c2nc(OCC34COC(C3)C4)nc3c(F)c(-c4ccc(F)c5sc(N)c(C#N)c45)c(Cl)c(c23)OCC2CC21]

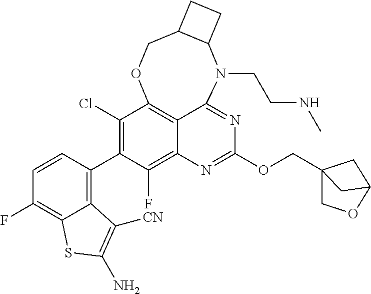 [SMILES: CNCCN1c2nc(OCC34COC(C3)C4)nc3c(F)c(-c4ccc(F)c5sc(N)c(C#N)c45)c(Cl)c(c23)OCC2CCC21]

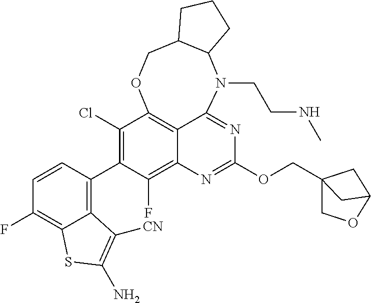 [SMILES: CNCCN1c2nc(OCC34COC(C3)C4)nc3c(F)c(-c4ccc(F)c5sc(N)c(C#N)c45)c(Cl)c(c23)OCC2CCCC21]

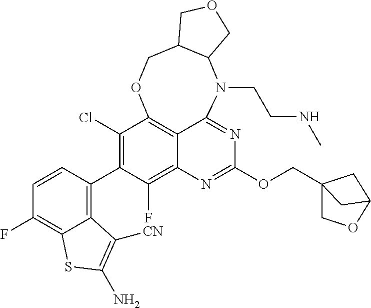 [SMILES: CNCCN1c2nc(OCC34COC(C3)C4)nc3c(F)c(-c4ccc(F)c5sc(N)c(C#N)c45)c(Cl)c(c23)OCC2COCC21]

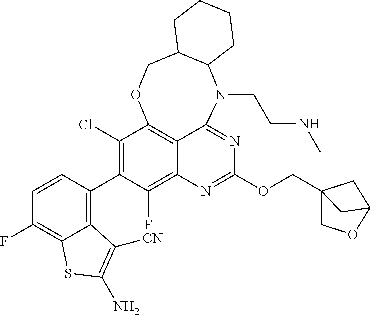 [SMILES: CNCCN1c2nc(OCC34COC(C3)C4)nc3c(F)c(-c4ccc(F)c5sc(N)c(C#N)c45)c(Cl)c(c23)OCC2CCCCC21]

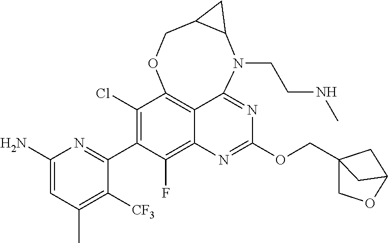 [SMILES: CNCCN1c2nc(OCC34COC(C3)C4)nc3c(F)c(-c4nc(N)cc(C)c4C(F)(F)F)c(Cl)c(c23)OCC2CC21]

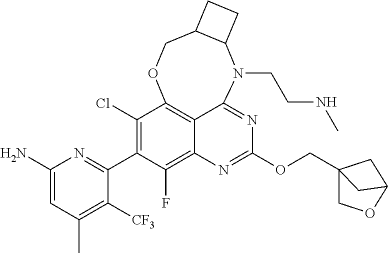 [SMILES: CNCCN1c2nc(OCC34COC(C3)C4)nc3c(F)c(-c4nc(N)cc(C)c4C(F)(F)F)c(Cl)c(c23)OCC2CCC21]

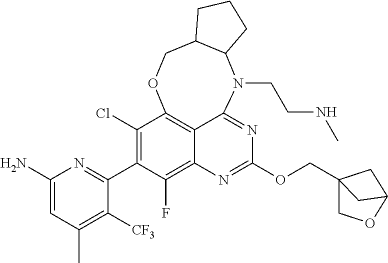 [SMILES: CNCCN1c2nc(OCC34COC(C3)C4)nc3c(F)c(-c4nc(N)cc(C)c4C(F)(F)F)c(Cl)c(c23)OCC2CCCC21]

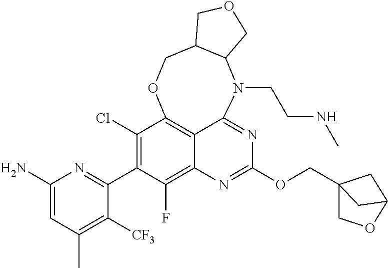 [SMILES: CNCCN1c2nc(OCC34COC(C3)C4)nc3c(F)c(-c4nc(N)cc(C)c4C(F)(F)F)c(Cl)c(c23)OCC2COCC21]

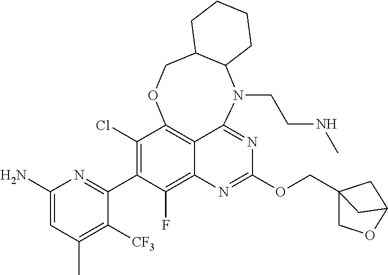 [SMILES: CNCCN1c2nc(OCC34COC(C3)C4)nc3c(F)c(-c4nc(N)cc(C)c4C(F)(F)F)c(Cl)c(c23)OCC2CCCCC21]

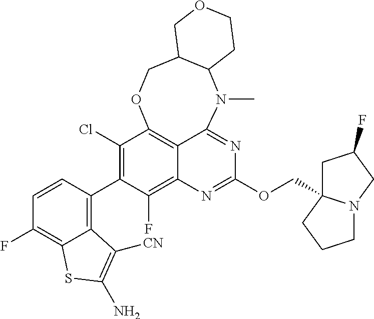 [SMILES: CN1c2nc(OC[C@@]34CCCN3C[C@H](F)C4)nc3c(F)c(-c4ccc(F)c5sc(N)c(C#N)c45)c(Cl)c(c23)OCC2COCCC21]

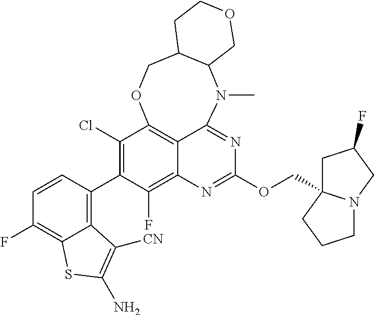 [SMILES: CN1c2nc(OC[C@@]34CCCN3C[C@H](F)C4)nc3c(F)c(-c4ccc(F)c5sc(N)c(C#N)c45)c(Cl)c(c23)OCC2CCOCC21]

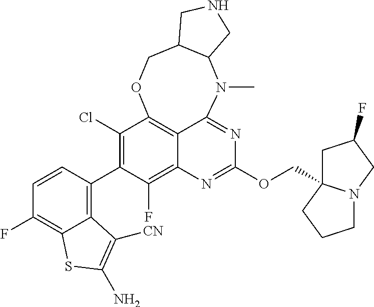 [SMILES: CN1c2nc(OC[C@@]34CCCN3C[C@H](F)C4)nc3c(F)c(-c4ccc(F)c5sc(N)c(C#N)c45)c(Cl)c(c23)OCC2CNCC21]

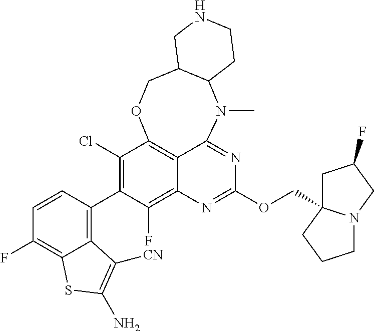 [SMILES: CN1c2nc(OC[C@@]34CCCN3C[C@H](F)C4)nc3c(F)c(-c4ccc(F)c5sc(N)c(C#N)c45)c(Cl)c(c23)OCC2CNCCC21]

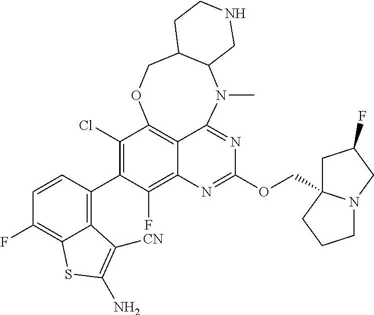 [SMILES: CN1c2nc(OC[C@@]34CCCN3C[C@H](F)C4)nc3c(F)c(-c4ccc(F)c5sc(N)c(C#N)c45)c(Cl)c(c23)OCC2CCNCC21]

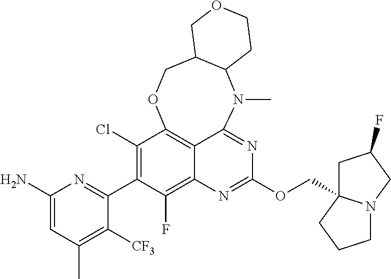 [SMILES: Cc1cc(N)nc(-c2c(Cl)c3c4c(nc(OC[C@@]56CCCN5C[C@H](F)C6)nc4c2F)N(C)C2CCOCC2CO3)c1C(F)(F)F]

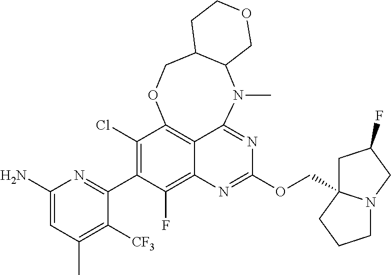 [SMILES: Cc1cc(N)nc(-c2c(Cl)c3c4c(nc(OC[C@@]56CCCN5C[C@H](F)C6)nc4c2F)N(C)C2COCCC2CO3)c1C(F)(F)F]

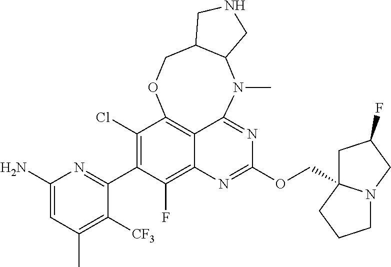 [SMILES: Cc1cc(N)nc(-c2c(Cl)c3c4c(nc(OC[C@@]56CCCN5C[C@H](F)C6)nc4c2F)N(C)C2CNCC2CO3)c1C(F)(F)F]

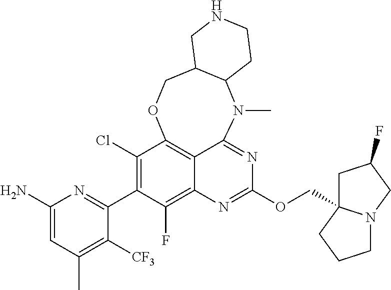 [SMILES: Cc1cc(N)nc(-c2c(Cl)c3c4c(nc(OC[C@@]56CCCN5C[C@H](F)C6)nc4c2F)N(C)C2CCNCC2CO3)c1C(F)(F)F]

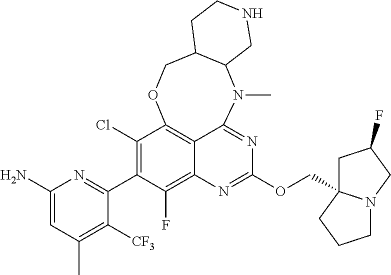 [SMILES: Cc1cc(N)nc(-c2c(Cl)c3c4c(nc(OC[C@@]56CCCN5C[C@H](F)C6)nc4c2F)N(C)C2CNCCC2CO3)c1C(F)(F)F]

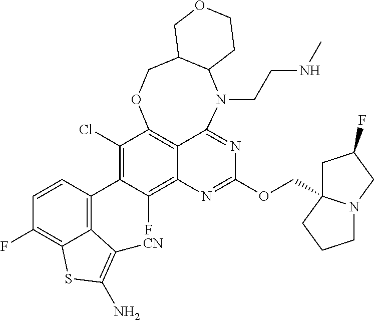 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC2COCCC21]

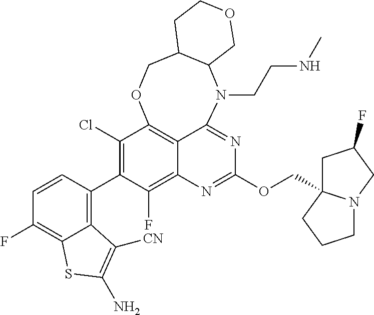 [SMILES: CNCCN1c2nc(OC[C@@]34CCCN3C[C@H](F)C4)nc3c(F)c(-c4ccc(F)c5sc(N)c(C#N)c45)c(Cl)c(c23)OCC2CCOCC21]

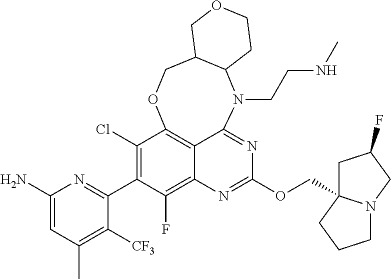 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2COCCC21]

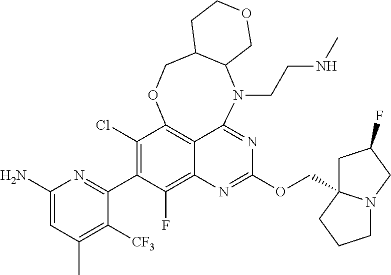 [SMILES: CNCCN1c2nc(OC[C@@]34CCCN3C[C@H](F)C4)nc3c(F)c(-c4nc(N)cc(C)c4C(F)(F)F)c(Cl)c(c23)OCC2CCOCC21]

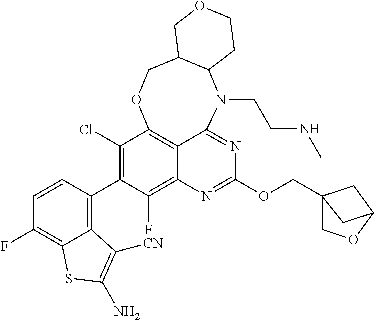 [SMILES: CNCCN1c2nc(OCC34COC(C3)C4)nc3c(F)c(-c4ccc(F)c5sc(N)c(C#N)c45)c(Cl)c(c23)OCC2COCCC21]

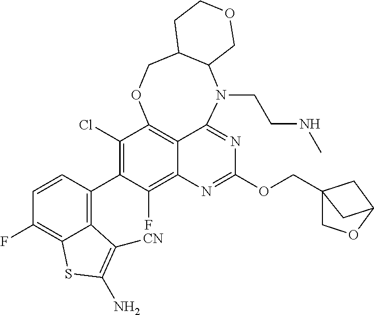 [SMILES: CNCCN1c2nc(OCC34COC(C3)C4)nc3c(F)c(-c4ccc(F)c5sc(N)c(C#N)c45)c(Cl)c(c23)OCC2CCOCC21]

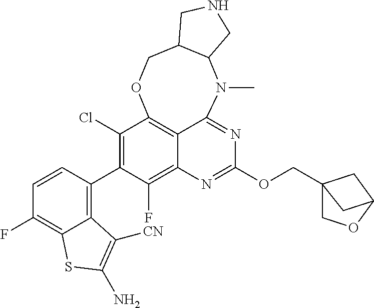 [SMILES: CN1c2nc(OCC34COC(C3)C4)nc3c(F)c(-c4ccc(F)c5sc(N)c(C#N)c45)c(Cl)c(c23)OCC2CNCC21]

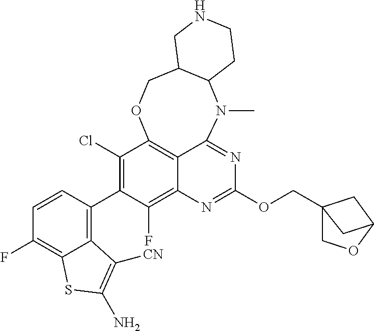 [SMILES: CN1c2nc(OCC34COC(C3)C4)nc3c(F)c(-c4ccc(F)c5sc(N)c(C#N)c45)c(Cl)c(c23)OCC2CNCCC21]

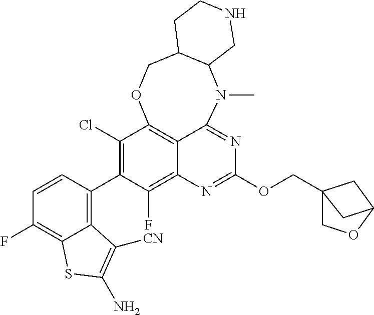 [SMILES: CN1c2nc(OCC34COC(C3)C4)nc3c(F)c(-c4ccc(F)c5sc(N)c(C#N)c45)c(Cl)c(c23)OCC2CCNCC21]

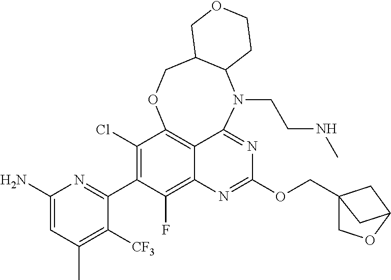 [SMILES: CNCCN1c2nc(OCC34COC(C3)C4)nc3c(F)c(-c4nc(N)cc(C)c4C(F)(F)F)c(Cl)c(c23)OCC2COCCC21]

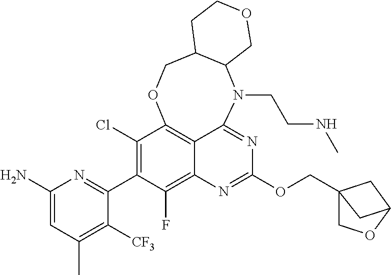 [SMILES: CNCCN1c2nc(OCC34COC(C3)C4)nc3c(F)c(-c4nc(N)cc(C)c4C(F)(F)F)c(Cl)c(c23)OCC2CCOCC21]

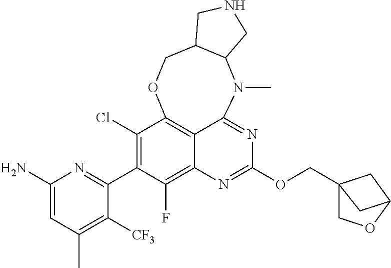 [SMILES: Cc1cc(N)nc(-c2c(Cl)c3c4c(nc(OCC56COC(C5)C6)nc4c2F)N(C)C2CNCC2CO3)c1C(F)(F)F]

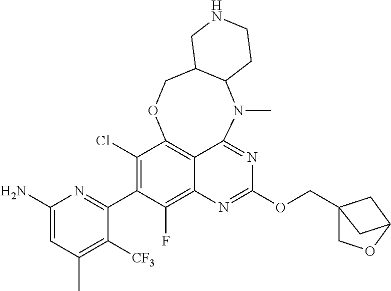 [SMILES: Cc1cc(N)nc(-c2c(Cl)c3c4c(nc(OCC56COC(C5)C6)nc4c2F)N(C)C2CCNCC2CO3)c1C(F)(F)F]

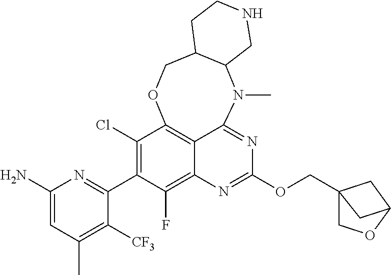 [SMILES: Cc1cc(N)nc(-c2c(Cl)c3c4c(nc(OCC56COC(C5)C6)nc4c2F)N(C)C2CNCCC2CO3)c1C(F)(F)F]

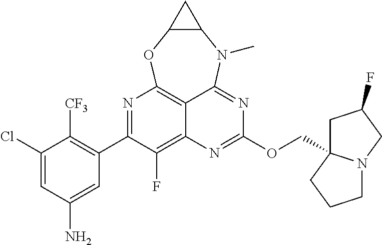 [SMILES: CN1c2nc(OC[C@@]34CCCN3C[C@H](F)C4)nc3c(F)c(-c4cc(N)cc(Cl)c4C(F)(F)F)nc(c23)OC2CC21]

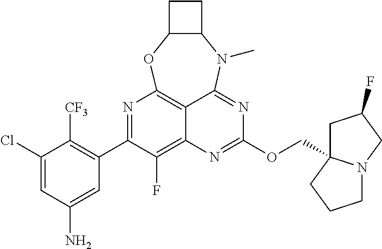 [SMILES: CN1c2nc(OC[C@@]34CCCN3C[C@H](F)C4)nc3c(F)c(-c4cc(N)cc(Cl)c4C(F)(F)F)nc(c23)OC2CCC21]

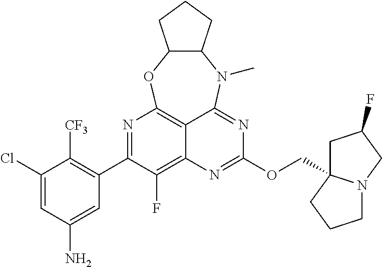 [SMILES: CN1c2nc(OC[C@@]34CCCN3C[C@H](F)C4)nc3c(F)c(-c4cc(N)cc(Cl)c4C(F)(F)F)nc(c23)OC2CCCC21]

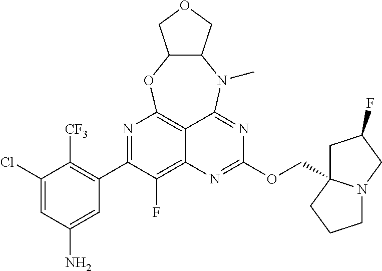 [SMILES: CN1c2nc(OC[C@@]34CCCN3C[C@H](F)C4)nc3c(F)c(-c4cc(N)cc(Cl)c4C(F)(F)F)nc(c23)OC2COCC21]

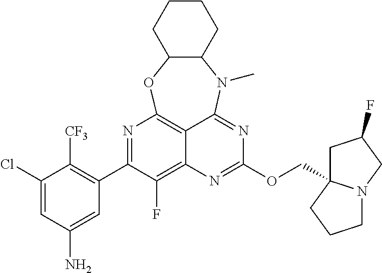 [SMILES: CN1c2nc(OC[C@@]34CCCN3C[C@H](F)C4)nc3c(F)c(-c4cc(N)cc(Cl)c4C(F)(F)F)nc(c23)OC2CCCCC21]

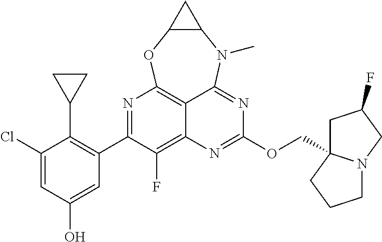 [SMILES: CN1c2nc(OC[C@@]34CCCN3C[C@H](F)C4)nc3c(F)c(-c4cc(O)cc(Cl)c4C4CC4)nc(c23)OC2CC21]

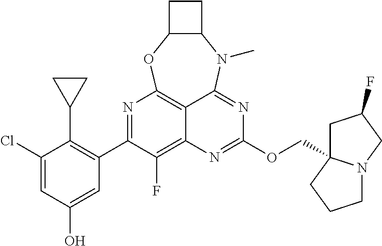 [SMILES: CN1c2nc(OC[C@@]34CCCN3C[C@H](F)C4)nc3c(F)c(-c4cc(O)cc(Cl)c4C4CC4)nc(c23)OC2CCC21]

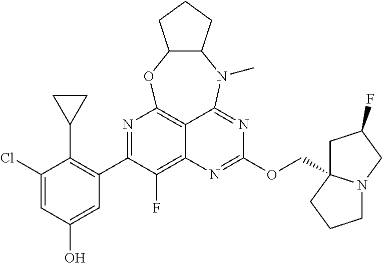 [SMILES: CN1c2nc(OC[C@@]34CCCN3C[C@H](F)C4)nc3c(F)c(-c4cc(O)cc(Cl)c4C4CC4)nc(c23)OC2CCCC21]

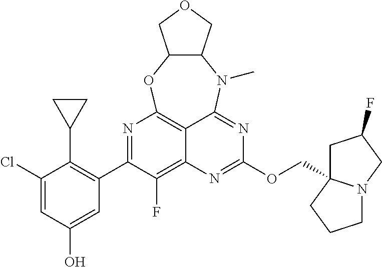 [SMILES: CN1c2nc(OC[C@@]34CCCN3C[C@H](F)C4)nc3c(F)c(-c4cc(O)cc(Cl)c4C4CC4)nc(c23)OC2COCC21]

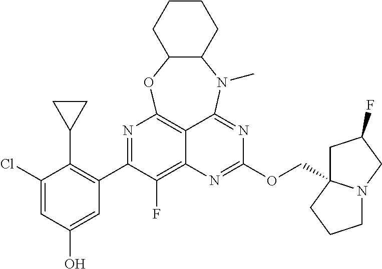 [SMILES: CN1c2nc(OC[C@@]34CCCN3C[C@H](F)C4)nc3c(F)c(-c4cc(O)cc(Cl)c4C4CC4)nc(c23)OC2CCCCC21]

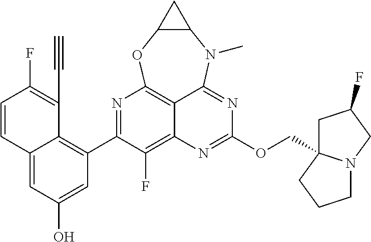 [SMILES: C#Cc1c(F)ccc2cc(O)cc(-c3nc4c5c(nc(OC[C@@]67CCCN6C[C@H](F)C7)nc5c3F)N(C)C3CC3O4)c12]

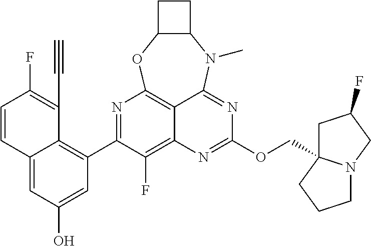 [SMILES: C#Cc1c(F)ccc2cc(O)cc(-c3nc4c5c(nc(OC[C@@]67CCCN6C[C@H](F)C7)nc5c3F)N(C)C3CCC3O4)c12]

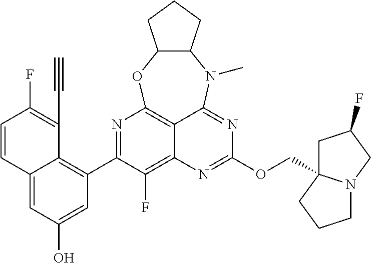 [SMILES: C#Cc1c(F)ccc2cc(O)cc(-c3nc4c5c(nc(OC[C@@]67CCCN6C[C@H](F)C7)nc5c3F)N(C)C3CCCC3O4)c12]

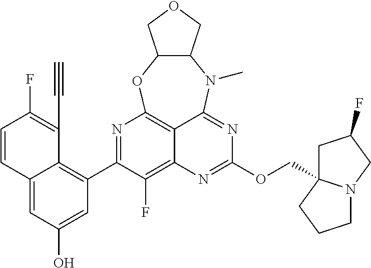 [SMILES: C#Cc1c(F)ccc2cc(O)cc(-c3nc4c5c(nc(OC[C@@]67CCCN6C[C@H](F)C7)nc5c3F)N(C)C3COCC3O4)c12]

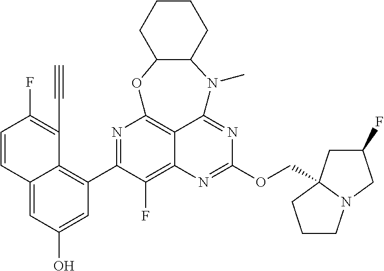 [SMILES: C#Cc1c(F)ccc2cc(O)cc(-c3nc4c5c(nc(OC[C@@]67CCCN6C[C@H](F)C7)nc5c3F)N(C)C3CCCCC3O4)c12]

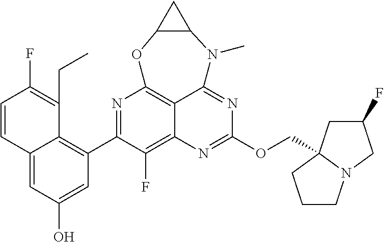 [SMILES: CCc1c(F)ccc2cc(O)cc(-c3nc4c5c(nc(OC[C@@]67CCCN6C[C@H](F)C7)nc5c3F)N(C)C3CC3O4)c12]

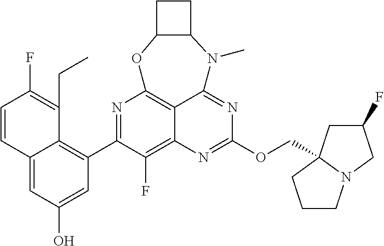 [SMILES: CCc1c(F)ccc2cc(O)cc(-c3nc4c5c(nc(OC[C@@]67CCCN6C[C@H](F)C7)nc5c3F)N(C)C3CCC3O4)c12]

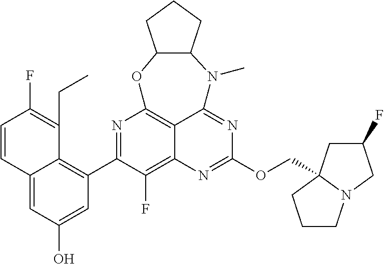 [SMILES: CCc1c(F)ccc2cc(O)cc(-c3nc4c5c(nc(OC[C@@]67CCCN6C[C@H](F)C7)nc5c3F)N(C)C3CCCC3O4)c12]

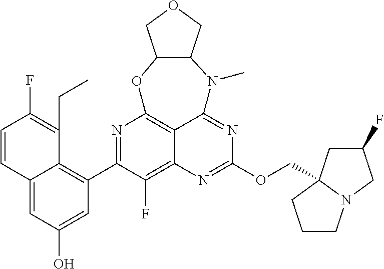 [SMILES: CCc1c(F)ccc2cc(O)cc(-c3nc4c5c(nc(OC[C@@]67CCCN6C[C@H](F)C7)nc5c3F)N(C)C3COCC3O4)c12]

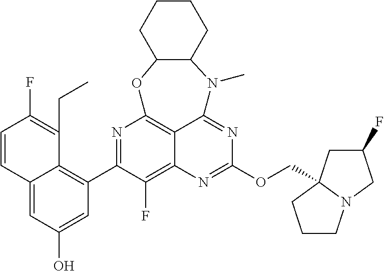 [SMILES: CCc1c(F)ccc2cc(O)cc(-c3nc4c5c(nc(OC[C@@]67CCCN6C[C@H](F)C7)nc5c3F)N(C)C3CCCCC3O4)c12]

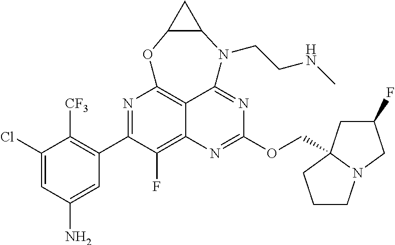 [SMILES: CNCCN1c2nc(OC[C@@]34CCCN3C[C@H](F)C4)nc3c(F)c(-c4cc(N)cc(Cl)c4C(F)(F)F)nc(c23)OC2CC21]

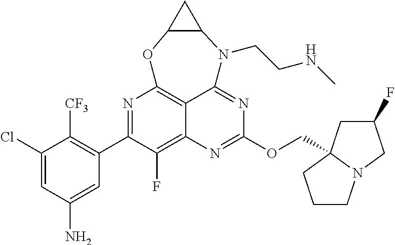 [SMILES: CNCCN1c2nc(OC[C@@]34CCCN3C[C@H](F)C4)nc3c(F)c(-c4cc(N)cc(Cl)c4C(F)(F)F)nc(c23)OC2CC21]

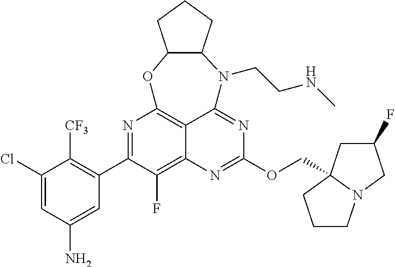 [SMILES: CNCCN1c2nc(OC[C@@]34CCCN3C[C@H](F)C4)nc3c(F)c(-c4cc(N)cc(Cl)c4C(F)(F)F)nc(c23)OC2CCCC21]

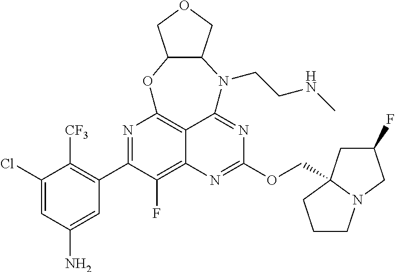 [SMILES: CNCCN1c2nc(OC[C@@]34CCCN3C[C@H](F)C4)nc3c(F)c(-c4cc(N)cc(Cl)c4C(F)(F)F)nc(c23)OC2COCC21]

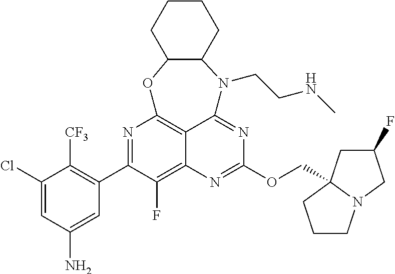 [SMILES: CNCCN1c2nc(OC[C@@]34CCCN3C[C@H](F)C4)nc3c(F)c(-c4cc(N)cc(Cl)c4C(F)(F)F)nc(c23)OC2CCCCC21]

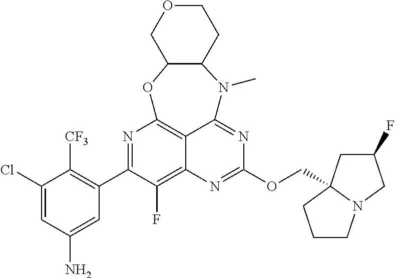 [SMILES: CN1c2nc(OC[C@@]34CCCN3C[C@H](F)C4)nc3c(F)c(-c4cc(N)cc(Cl)c4C(F)(F)F)nc(c23)OC2COCCC21]

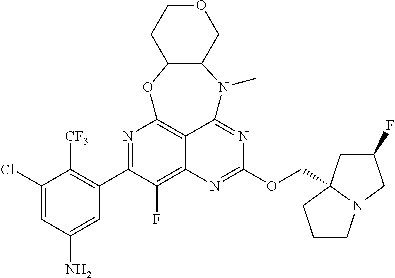 [SMILES: CN1c2nc(OC[C@@]34CCCN3C[C@H](F)C4)nc3c(F)c(-c4cc(N)cc(Cl)c4C(F)(F)F)nc(c23)OC2CCOCC21]

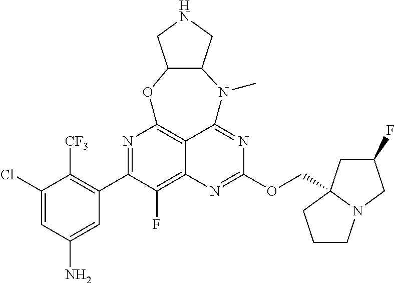 [SMILES: CN1c2nc(OC[C@@]34CCCN3C[C@H](F)C4)nc3c(F)c(-c4cc(N)cc(Cl)c4C(F)(F)F)nc(c23)OC2CNCC21]

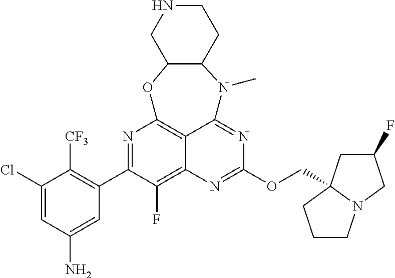 [SMILES: CN1c2nc(OC[C@@]34CCCN3C[C@H](F)C4)nc3c(F)c(-c4cc(N)cc(Cl)c4C(F)(F)F)nc(c23)OC2CNCCC21]

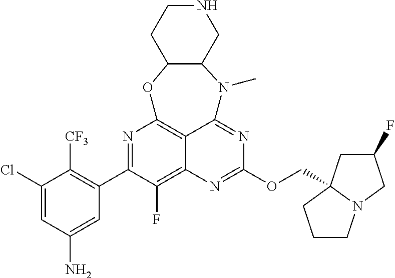 [SMILES: CN1c2nc(OC[C@@]34CCCN3C[C@H](F)C4)nc3c(F)c(-c4cc(N)cc(Cl)c4C(F)(F)F)nc(c23)OC2CCNCC21]

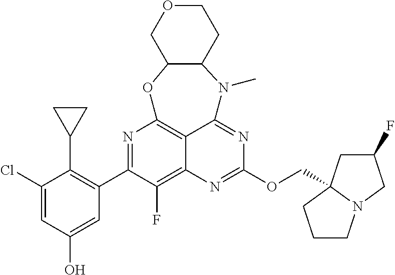 [SMILES: CN1c2nc(OC[C@@]34CCCN3C[C@H](F)C4)nc3c(F)c(-c4cc(O)cc(Cl)c4C4CC4)nc(c23)OC2COCCC21]

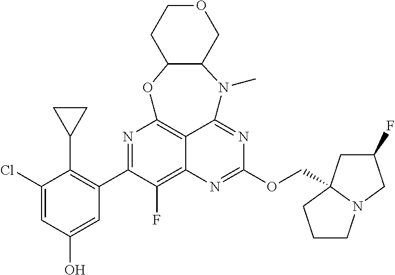 [SMILES: CN1c2nc(OC[C@@]34CCCN3C[C@H](F)C4)nc3c(F)c(-c4cc(O)cc(Cl)c4C4CC4)nc(c23)OC2CCOCC21]

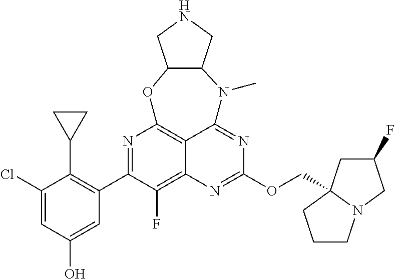 [SMILES: CN1c2nc(OC[C@@]34CCCN3C[C@H](F)C4)nc3c(F)c(-c4cc(O)cc(Cl)c4C4CC4)nc(c23)OC2CNCC21]

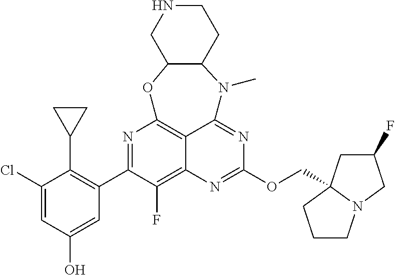 [SMILES: CN1c2nc(OC[C@@]34CCCN3C[C@H](F)C4)nc3c(F)c(-c4cc(O)cc(Cl)c4C4CC4)nc(c23)OC2CNCCC21]

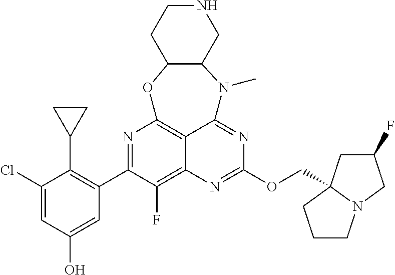 [SMILES: CN1c2nc(OC[C@@]34CCCN3C[C@H](F)C4)nc3c(F)c(-c4cc(O)cc(Cl)c4C4CC4)nc(c23)OC2CCNCC21]

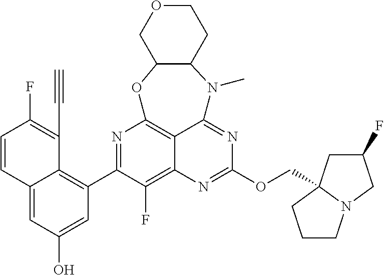 [SMILES: C#Cc1c(F)ccc2cc(O)cc(-c3nc4c5c(nc(OC[C@@]67CCCN6C[C@H](F)C7)nc5c3F)N(C)C3CCOCC3O4)c12]

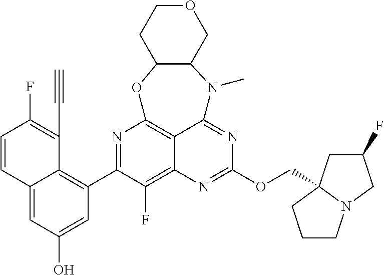 [SMILES: C#Cc1c(F)ccc2cc(O)cc(-c3nc4c5c(nc(OC[C@@]67CCCN6C[C@H](F)C7)nc5c3F)N(C)C3COCCC3O4)c12]

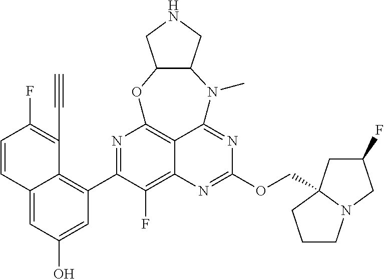 [SMILES: C#Cc1c(F)ccc2cc(O)cc(-c3nc4c5c(nc(OC[C@@]67CCCN6C[C@H](F)C7)nc5c3F)N(C)C3CNCC3O4)c12]

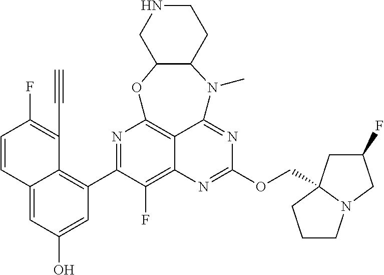 [SMILES: C#Cc1c(F)ccc2cc(O)cc(-c3nc4c5c(nc(OC[C@@]67CCCN6C[C@H](F)C7)nc5c3F)N(C)C3CCNCC3O4)c12]

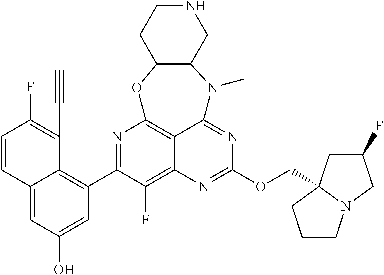 [SMILES: C#Cc1c(F)ccc2cc(O)cc(-c3nc4c5c(nc(OC[C@@]67CCCN6C[C@H](F)C7)nc5c3F)N(C)C3CNCCC3O4)c12]

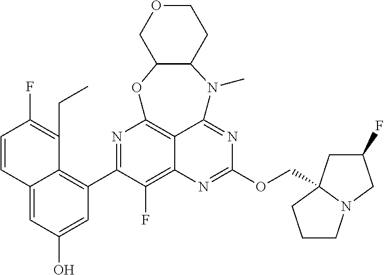 [SMILES: CCc1c(F)ccc2cc(O)cc(-c3nc4c5c(nc(OC[C@@]67CCCN6C[C@H](F)C7)nc5c3F)N(C)C3CCOCC3O4)c12]

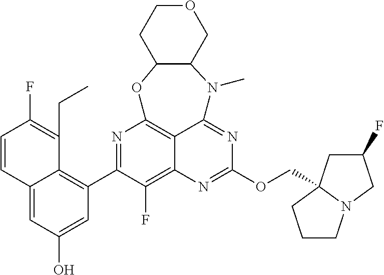 [SMILES: CCc1c(F)ccc2cc(O)cc(-c3nc4c5c(nc(OC[C@@]67CCCN6C[C@H](F)C7)nc5c3F)N(C)C3COCCC3O4)c12]

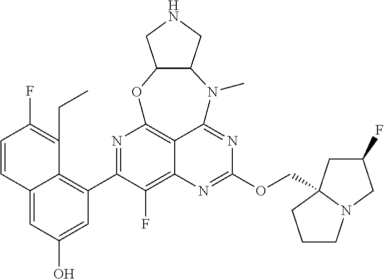 [SMILES: CCc1c(F)ccc2cc(O)cc(-c3nc4c5c(nc(OC[C@@]67CCCN6C[C@H](F)C7)nc5c3F)N(C)C3CNCC3O4)c12]

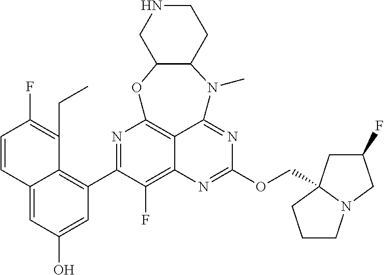 [SMILES: CCc1c(F)ccc2cc(O)cc(-c3nc4c5c(nc(OC[C@@]67CCCN6C[C@H](F)C7)nc5c3F)N(C)C3CCNCC3O4)c12]

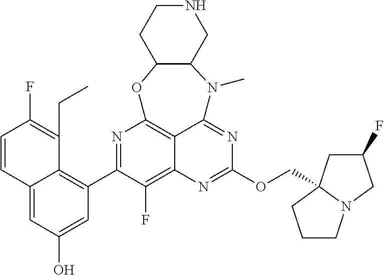 [SMILES: CCc1c(F)ccc2cc(O)cc(-c3nc4c5c(nc(OC[C@@]67CCCN6C[C@H](F)C7)nc5c3F)N(C)C3CNCCC3O4)c12]

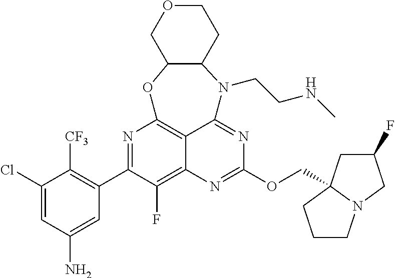 [SMILES: CNCCN1c2nc(OC[C@@]34CCCN3C[C@H](F)C4)nc3c(F)c(-c4cc(N)cc(Cl)c4C(F)(F)F)nc(c23)OC2COCCC21]

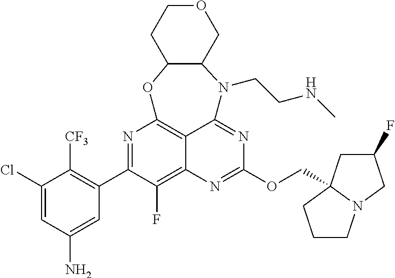 [SMILES: CNCCN1c2nc(OC[C@@]34CCCN3C[C@H](F)C4)nc3c(F)c(-c4cc(N)cc(Cl)c4C(F)(F)F)nc(c23)OC2CCOCC21]

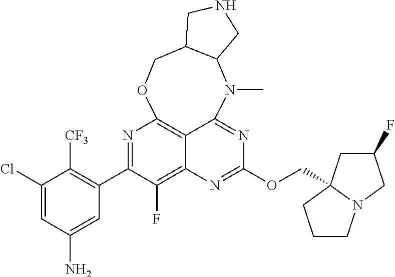 [SMILES: CN1c2nc(OC[C@@]34CCCN3C[C@H](F)C4)nc3c(F)c(-c4cc(N)cc(Cl)c4C(F)(F)F)nc(c23)OCC2CNCC21]

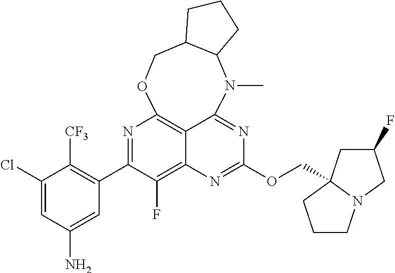 [SMILES: CN1c2nc(OC[C@@]34CCCN3C[C@H](F)C4)nc3c(F)c(-c4cc(N)cc(Cl)c4C(F)(F)F)nc(c23)OCC2CCCC21]

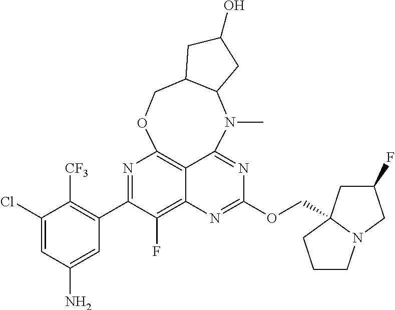 [SMILES: CN1c2nc(OC[C@@]34CCCN3C[C@H](F)C4)nc3c(F)c(-c4cc(N)cc(Cl)c4C(F)(F)F)nc(c23)OCC2CC(O)CC21]

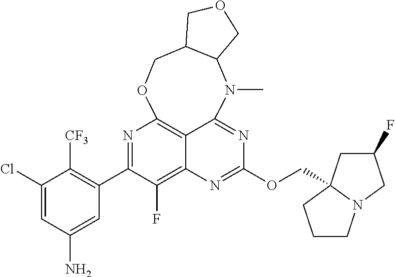 [SMILES: CN1c2nc(OC[C@@]34CCCN3C[C@H](F)C4)nc3c(F)c(-c4cc(N)cc(Cl)c4C(F)(F)F)nc(c23)OCC2COCC21]

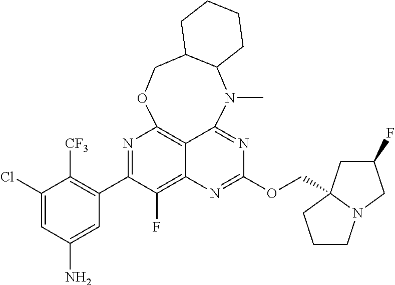 [SMILES: CN1c2nc(OC[C@@]34CCCN3C[C@H](F)C4)nc3c(F)c(-c4cc(N)cc(Cl)c4C(F)(F)F)nc(c23)OCC2CCCCC21]

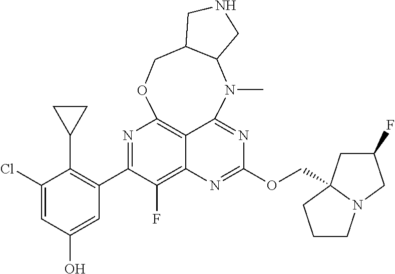 [SMILES: CN1c2nc(OC[C@@]34CCCN3C[C@H](F)C4)nc3c(F)c(-c4cc(O)cc(Cl)c4C4CC4)nc(c23)OCC2CNCC21]

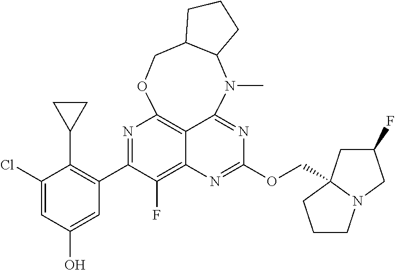 [SMILES: CN1c2nc(OC[C@@]34CCCN3C[C@H](F)C4)nc3c(F)c(-c4cc(O)cc(Cl)c4C4CC4)nc(c23)OCC2CCCC21]

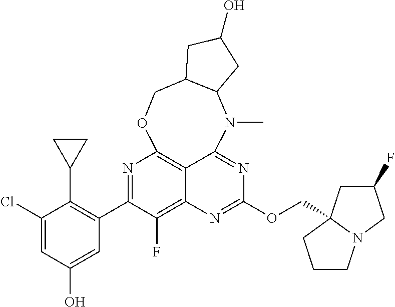 [SMILES: CN1c2nc(OC[C@@]34CCCN3C[C@H](F)C4)nc3c(F)c(-c4cc(O)cc(Cl)c4C4CC4)nc(c23)OCC2CC(O)CC21]

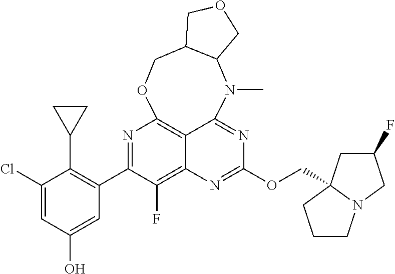 [SMILES: CN1c2nc(OC[C@@]34CCCN3C[C@H](F)C4)nc3c(F)c(-c4cc(O)cc(Cl)c4C4CC4)nc(c23)OCC2COCC21]

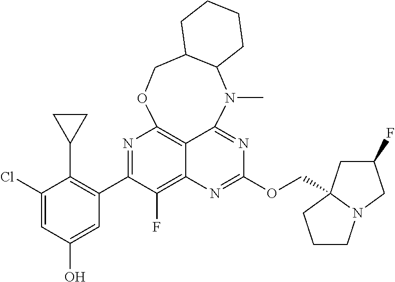 [SMILES: CN1c2nc(OC[C@@]34CCCN3C[C@H](F)C4)nc3c(F)c(-c4cc(O)cc(Cl)c4C4CC4)nc(c23)OCC2CCCCC21]

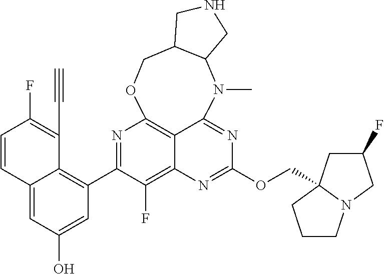 [SMILES: C#Cc1c(F)ccc2cc(O)cc(-c3nc4c5c(nc(OC[C@@]67CCCN6C[C@H](F)C7)nc5c3F)N(C)C3CNCC3CO4)c12]

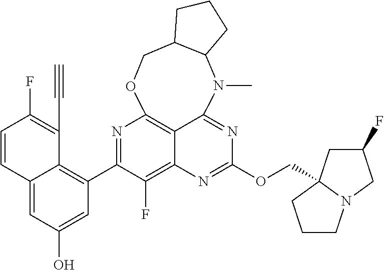 [SMILES: C#Cc1c(F)ccc2cc(O)cc(-c3nc4c5c(nc(OC[C@@]67CCCN6C[C@H](F)C7)nc5c3F)N(C)C3CCCC3CO4)c12]

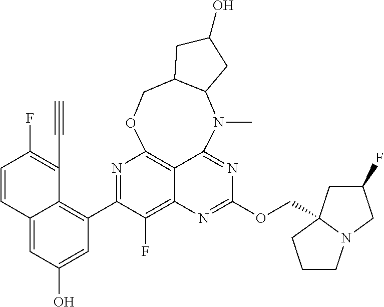 [SMILES: C#Cc1c(F)ccc2cc(O)cc(-c3nc4c5c(nc(OC[C@@]67CCCN6C[C@H](F)C7)nc5c3F)N(C)C3CC(O)CC3CO4)c12]

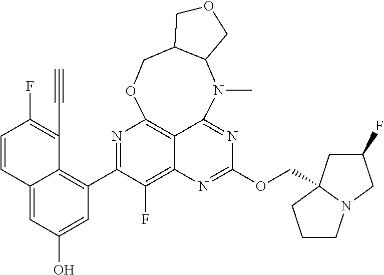 [SMILES: C#Cc1c(F)ccc2cc(O)cc(-c3nc4c5c(nc(OC[C@@]67CCCN6C[C@H](F)C7)nc5c3F)N(C)C3COCC3CO4)c12]

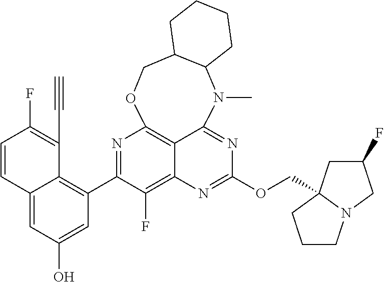 [SMILES: C#Cc1c(F)ccc2cc(O)cc(-c3nc4c5c(nc(OC[C@@]67CCCN6C[C@H](F)C7)nc5c3F)N(C)C3CCCCC3CO4)c12]

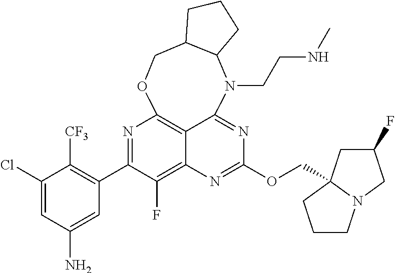 [SMILES: CNCCN1c2nc(OC[C@@]34CCCN3C[C@H](F)C4)nc3c(F)c(-c4cc(N)cc(Cl)c4C(F)(F)F)nc(c23)OCC2CCCC21]

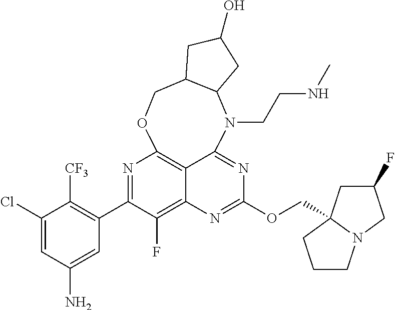 [SMILES: CNCCN1c2nc(OC[C@@]34CCCN3C[C@H](F)C4)nc3c(F)c(-c4cc(N)cc(Cl)c4C(F)(F)F)nc(c23)OCC2CC(O)CC21]

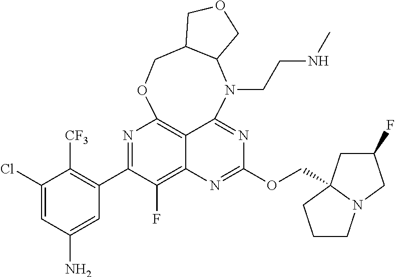 [SMILES: CNCCN1c2nc(OC[C@@]34CCCN3C[C@H](F)C4)nc3c(F)c(-c4cc(N)cc(Cl)c4C(F)(F)F)nc(c23)OCC2COCC21]

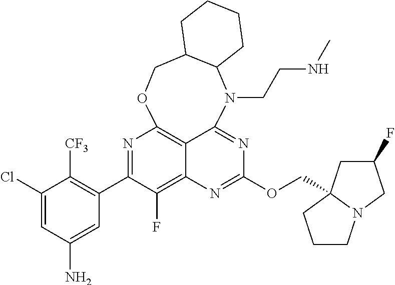 [SMILES: CNCCN1c2nc(OC[C@@]34CCCN3C[C@H](F)C4)nc3c(F)c(-c4cc(N)cc(Cl)c4C(F)(F)F)nc(c23)OCC2CCCCC21]

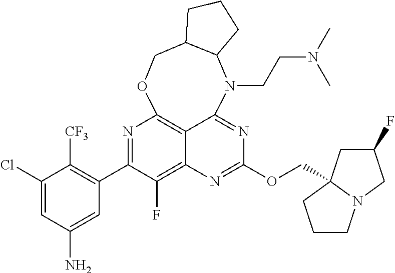 [SMILES: CN(C)CCN1c2nc(OC[C@@]34CCCN3C[C@H](F)C4)nc3c(F)c(-c4cc(N)cc(Cl)c4C(F)(F)F)nc(c23)OCC2CCCC21]

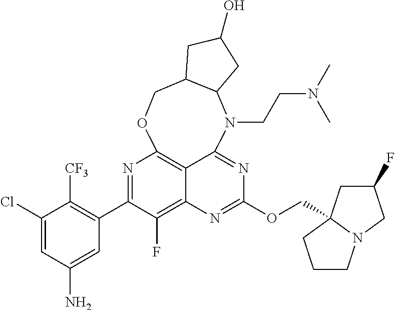 [SMILES: CN(C)CCN1c2nc(OC[C@@]34CCCN3C[C@H](F)C4)nc3c(F)c(-c4cc(N)cc(Cl)c4C(F)(F)F)nc(c23)OCC2CC(O)CC21]

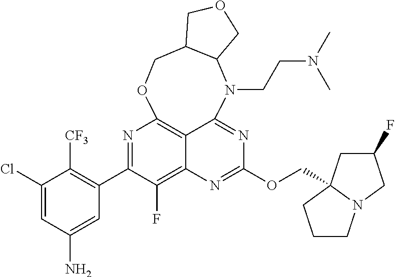 [SMILES: CN(C)CCN1c2nc(OC[C@@]34CCCN3C[C@H](F)C4)nc3c(F)c(-c4cc(N)cc(Cl)c4C(F)(F)F)nc(c23)OCC2COCC21]

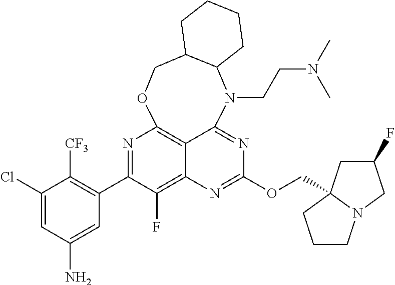 [SMILES: CN(C)CCN1c2nc(OC[C@@]34CCCN3C[C@H](F)C4)nc3c(F)c(-c4cc(N)cc(Cl)c4C(F)(F)F)nc(c23)OCC2CCCCC21]

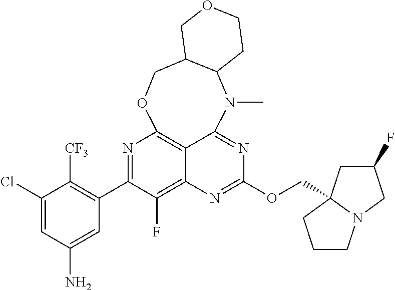 [SMILES: CN1c2nc(OC[C@@]34CCCN3C[C@H](F)C4)nc3c(F)c(-c4cc(N)cc(Cl)c4C(F)(F)F)nc(c23)OCC2COCCC21]

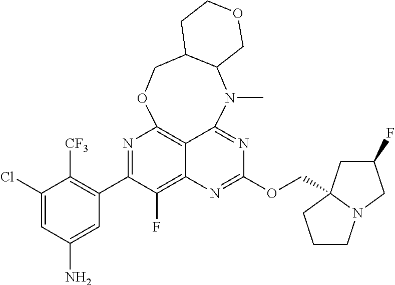 [SMILES: CN1c2nc(OC[C@@]34CCCN3C[C@H](F)C4)nc3c(F)c(-c4cc(N)cc(Cl)c4C(F)(F)F)nc(c23)OCC2CCOCC21]

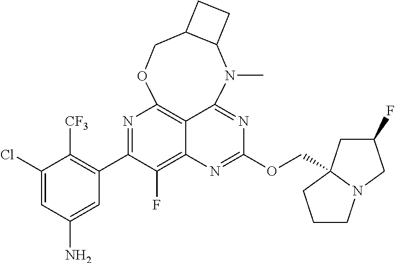 [SMILES: CN1c2nc(OC[C@@]34CCCN3C[C@H](F)C4)nc3c(F)c(-c4cc(N)cc(Cl)c4C(F)(F)F)nc(c23)OCC2CCC21]

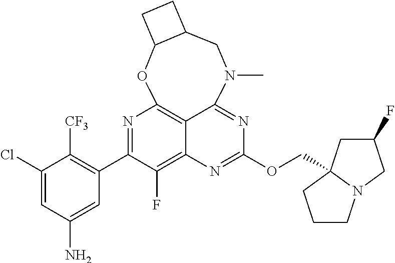 [SMILES: CN1CC2CCC2Oc2nc(-c3cc(N)cc(Cl)c3C(F)(F)F)c(F)c3nc(OC[C@@]45CCCN4C[C@H](F)C5)nc1c23]

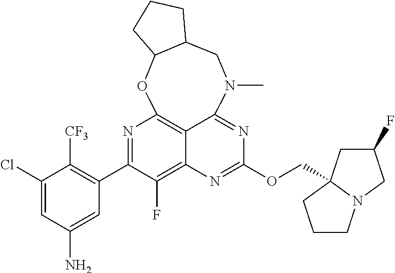 [SMILES: CN1CC2CCCC2Oc2nc(-c3cc(N)cc(Cl)c3C(F)(F)F)c(F)c3nc(OC[C@@]45CCCN4C[C@H](F)C5)nc1c23]

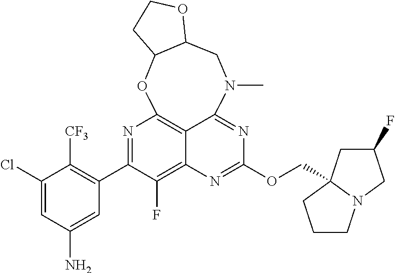 [SMILES: CN1CC2OCCC2Oc2nc(-c3cc(N)cc(Cl)c3C(F)(F)F)c(F)c3nc(OC[C@@]45CCCN4C[C@H](F)C5)nc1c23]

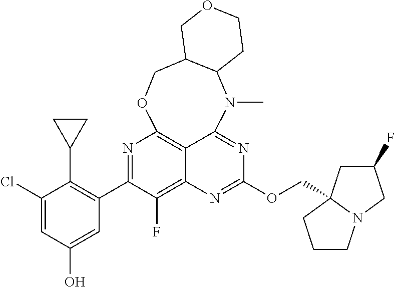 [SMILES: CN1c2nc(OC[C@@]34CCCN3C[C@H](F)C4)nc3c(F)c(-c4cc(O)cc(Cl)c4C4CC4)nc(c23)OCC2COCCC21]

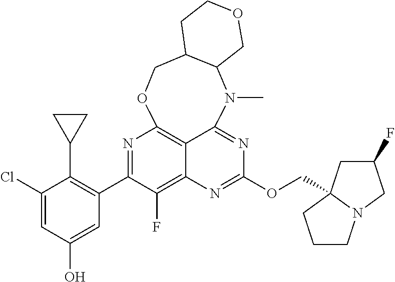 [SMILES: CN1c2nc(OC[C@@]34CCCN3C[C@H](F)C4)nc3c(F)c(-c4cc(O)cc(Cl)c4C4CC4)nc(c23)OCC2CCOCC21]

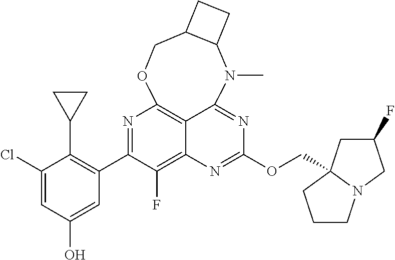 [SMILES: CN1c2nc(OC[C@@]34CCCN3C[C@H](F)C4)nc3c(F)c(-c4cc(O)cc(Cl)c4C4CC4)nc(c23)OCC2CCC21]

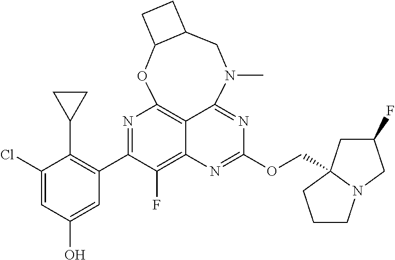 [SMILES: CN1CC2CCC2Oc2nc(-c3cc(O)cc(Cl)c3C3CC3)c(F)c3nc(OC[C@@]45CCCN4C[C@H](F)C5)nc1c23]

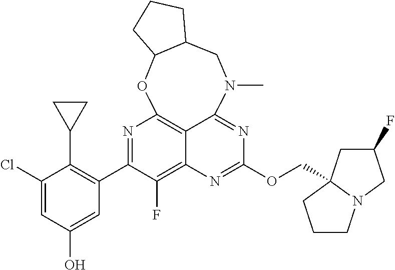 [SMILES: CN1CC2CCCC2Oc2nc(-c3cc(O)cc(Cl)c3C3CC3)c(F)c3nc(OC[C@@]45CCCN4C[C@H](F)C5)nc1c23]

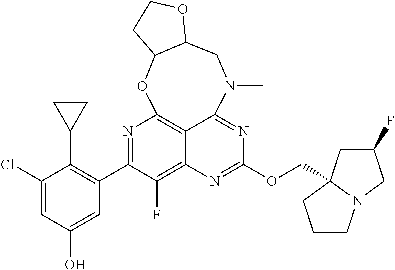 [SMILES: CN1CC2OCCC2Oc2nc(-c3cc(O)cc(Cl)c3C3CC3)c(F)c3nc(OC[C@@]45CCCN4C[C@H](F)C5)nc1c23]

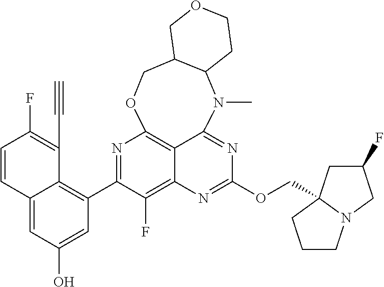 [SMILES: C#Cc1c(F)ccc2cc(O)cc(-c3nc4c5c(nc(OC[C@@]67CCCN6C[C@H](F)C7)nc5c3F)N(C)C3CCOCC3CO4)c12]

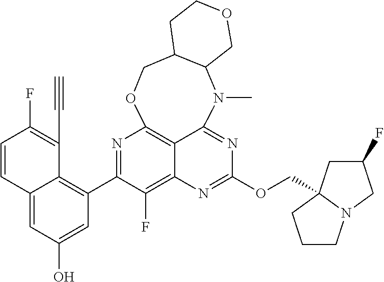 [SMILES: C#Cc1c(F)ccc2cc(O)cc(-c3nc4c5c(nc(OC[C@@]67CCCN6C[C@H](F)C7)nc5c3F)N(C)C3COCCC3CO4)c12]

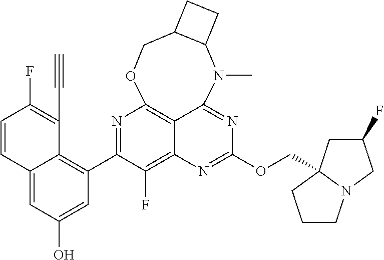 [SMILES: C#Cc1c(F)ccc2cc(O)cc(-c3nc4c5c(nc(OC[C@@]67CCCN6C[C@H](F)C7)nc5c3F)N(C)C3CCC3CO4)c12]

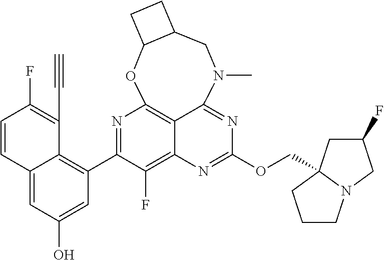 [SMILES: C#Cc1c(F)ccc2cc(O)cc(-c3nc4c5c(nc(OC[C@@]67CCCN6C[C@H](F)C7)nc5c3F)N(C)CC3CCC3O4)c12]

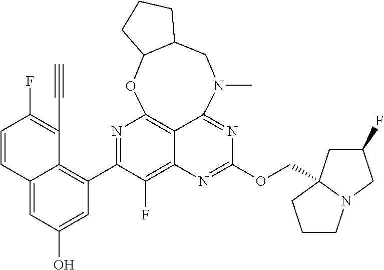 [SMILES: C#Cc1c(F)ccc2cc(O)cc(-c3nc4c5c(nc(OC[C@@]67CCCN6C[C@H](F)C7)nc5c3F)N(C)CC3CCCC3O4)c12]

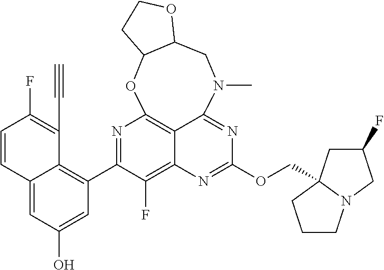 [SMILES: C#Cc1c(F)ccc2cc(O)cc(-c3nc4c5c(nc(OC[C@@]67CCCN6C[C@H](F)C7)nc5c3F)N(C)CC3OCCC3O4)c12]

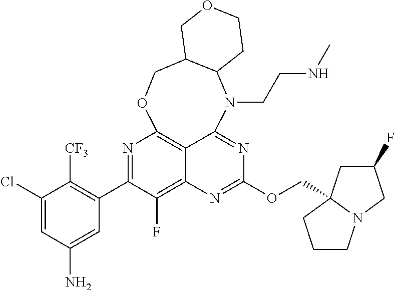 [SMILES: CNCCN1c2nc(OC[C@@]34CCCN3C[C@H](F)C4)nc3c(F)c(-c4cc(N)cc(Cl)c4C(F)(F)F)nc(c23)OCC2COCCC21]

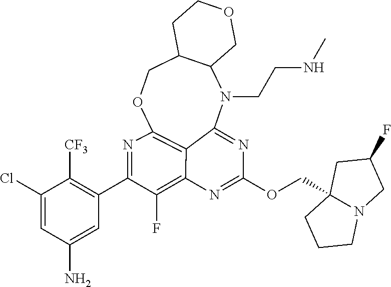 [SMILES: CNCCN1c2nc(OC[C@@]34CCCN3C[C@H](F)C4)nc3c(F)c(-c4cc(N)cc(Cl)c4C(F)(F)F)nc(c23)OCC2CCOCC21]

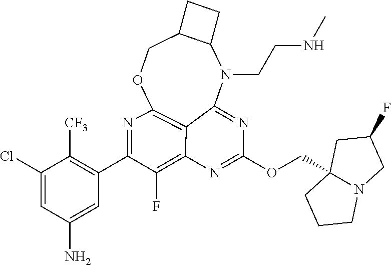 [SMILES: CNCCN1c2nc(OC[C@@]34CCCN3C[C@H](F)C4)nc3c(F)c(-c4cc(N)cc(Cl)c4C(F)(F)F)nc(c23)OCC2CCC21]

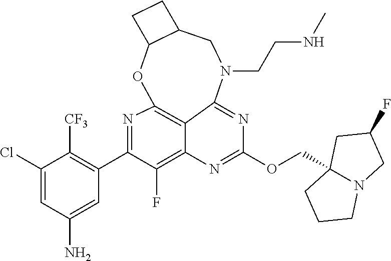 [SMILES: CNCCN1CC2CCC2Oc2nc(-c3cc(N)cc(Cl)c3C(F)(F)F)c(F)c3nc(OC[C@@]45CCCN4C[C@H](F)C5)nc1c23]

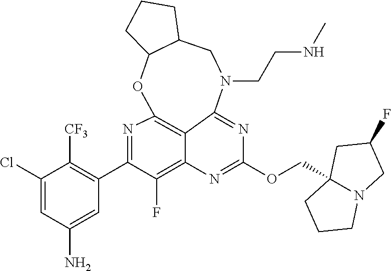 [SMILES: CNCCN1CC2CCCC2Oc2nc(-c3cc(N)cc(Cl)c3C(F)(F)F)c(F)c3nc(OC[C@@]45CCCN4C[C@H](F)C5)nc1c23]

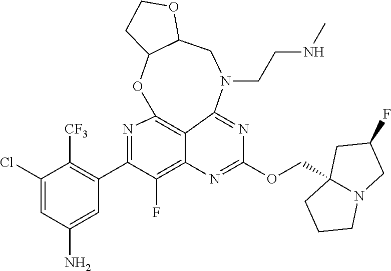 [SMILES: CNCCN1CC2OCCC2Oc2nc(-c3cc(N)cc(Cl)c3C(F)(F)F)c(F)c3nc(OC[C@@]45CCCN4C[C@H](F)C5)nc1c23]

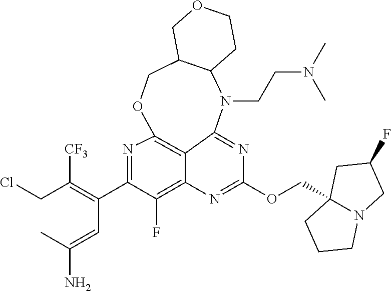 [SMILES: C/C(N)=C\C(=C(/CCl)C(F)(F)F)c1nc2c3c(nc(OC[C@@]45CCCN4C[C@H](F)C5)nc3c1F)N(CCN(C)C)C1CCOCC1CO2]

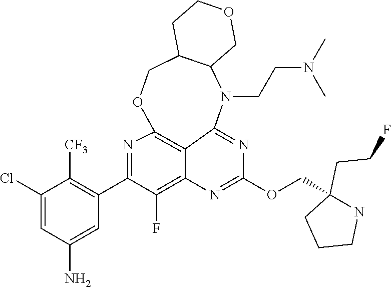

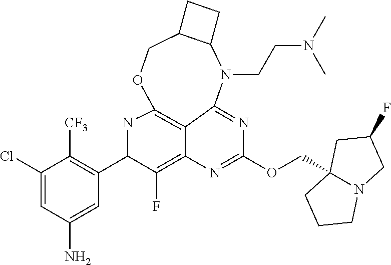 [SMILES: CN(C)CCN1c2nc(OC[C@@]34CCCN3C[C@H](F)C4)nc3c2=C(NC(c2cc(N)cc(Cl)c2C(F)(F)F)C=3F)OCC2CCC21]

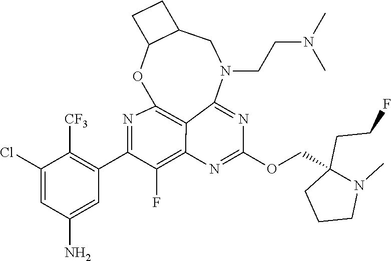

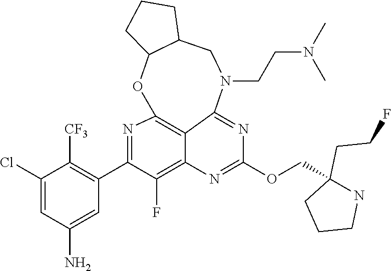

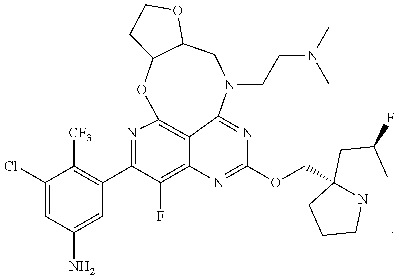 [SMILES: C[C@H](F)C[C@]1(COc2nc3c4c(nc(-c5cc(N)cc(Cl)c5C(F)(F)F)c(F)c4n2)OC2CCOC2CN3CCN(C)C)CCCN1]

Aspect 17: In some embodiments, the compounds provided herein has the following formula:

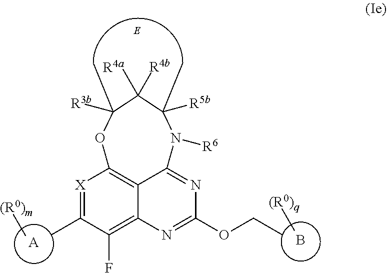

and pharmaceutically acceptable salts, tautomers, stereoisomers, enantiomers, atropisomers, isotopologues, and prodrugs thereof,
wherein:
ring E is unsubstituted or substituted 4-membered to 6-membered cycloalkyl or unsubstituted or substituted 4-membered to 6-membered heterocyclyl.

In one embodiment, ring E has one oxygen as the heteroatom.

In some embodiments, the compounds provided herein has the following formula:

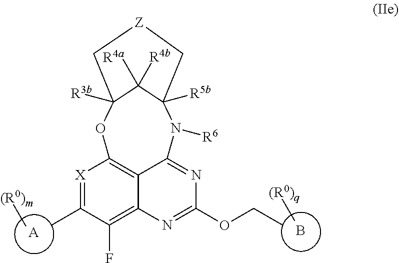

and pharmaceutically acceptable salts, tautomers, stereoisomers, enantiomers, atropisomers, isotopologues, and prodrugs thereof,
wherein:
Z is CH₂, NH, or O; and
R^{c} is H, halogen, amino, —CN, —OH, unsubstituted or substituted C₁₋₄ alkyl, unsubstituted or

substituted C₁₋₄ alkoxy, or unsubstituted or substituted C₁₋₄ alkylamino.

In one embodiment, Z is CH₂. In one embodiment, Z is O. In one embodiment, Z is NH.

Aspect 18: In some embodiments, the compounds provided herein have the following

formula:

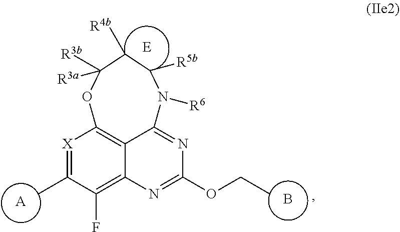

and pharmaceutically acceptable salts, tautomers, stereoisomers, enantiomers, atropisomers, isotopologues, and prodrugs thereof,
wherein:
ring E is unsubstituted or substituted cyclopentyl ring, unsubstituted or substituted cyclohexyl ring, unsubstituted or substituted cycloheptyl ring, unsubstituted or substituted tetrahydrofuranyl ring, unsubstituted or substituted tetrahydropyranyl ring, or unsubstituted or substituted oxepanyl ring.

In one emobidment, ring E is unsubstituted or substituted cyclopentyl ring. In one emobidment, ring E is unsubstituted or substituted tetrahydrofuranyl ring. In one emobidment, ring E is optionally substituted with OH, CN, unsubstituted or substituted amino, or unsubstituted or substituted C₁₋₄ alkyl. In one embodiment, ring E is optionally substituted with OH. In one embodiment, ring E is unsubstituted tetrahydrofuranyl ring. In one embodiment, ring E is unsubstituted tetrahydropyranyl ring. In one embodiment, ring E is unsubstituted oxepanyl ring.

In one embodiment, each of R^{3a}, R^{3b}, R^{4b}, and R^{5b}, independently, is H, OH, CN, halogen, unsubstituted or substituted amino, or unsubstituted or substituted C₁₋₄ alkyl. In one embodiment, each of R^{3a}, R^{3b}, R^{4b}, and R^{5b}, independently, is H, or unsubstituted or substituted methyl. In one embodiment, each of R^{3a}, R^{3b}, R^{4b}, and R^{5b}, independently, is H, OH, CN, halogen, or unsubstituted methyl. In one embodiment, each of R^{3a}, R^{3b}, R^{4b}, and R^{5b}, independently, is H, F, CN, or unsubstituted methyl. In one embodiment, each of R^{3a}, R^{3b}, R^{4b}, and R^{5b}, independently, is H.

In one embodiment, R⁶ is H, unsubstituted or substituted C₁₋₄ alkyl, unsubstituted or substituted C₃₋₄ cycloalkyl, or unsubstituted or substituted 3-membered to 4-membered heterocyclyl. In one embodiment, R⁶ is methyl, ethyl, 2-methylamino-ethyl, or cyclopropyl. In one embodiment, R⁶ is methyl.

In one embodiment, X is N. In one embodiment, X is CCF₃. In one embodiment, X is CH.

In one embodiment, ring A is

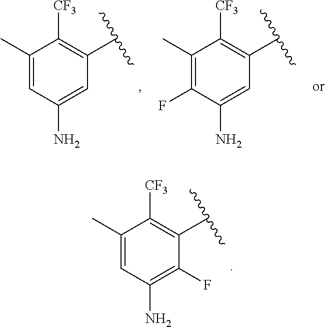

In one embodiment, ring A is

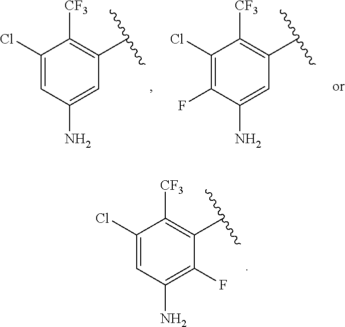 [SMILES: *c1cc(N)cc(Cl)c1C(F)(F)F ; *c1cc(N)c(F)c(Cl)c1C(F)(F)F ; *c1c(F)c(N)cc(Cl)c1C(F)(F)F]

In one embodiment, ring A is

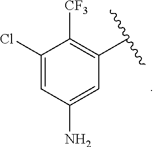 [SMILES: *c1cc(N)cc(Cl)c1C(F)(F)F]

In one embodiment, ring A is

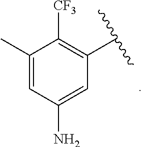 [SMILES: *c1cc(N)cc(C)c1C(F)(F)F]

one embodiment, ring A is

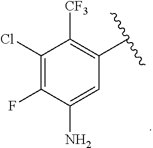 [SMILES: *c1cc(N)c(F)c(Cl)c1C(F)(F)F]

In one embodiment, ring B is

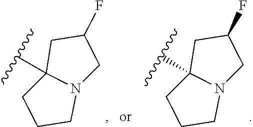 [SMILES: *C12CCCN1CC(F)C2 ; *[C@@]12CCCN1C[C@H](F)C2]

In one embodiment, ring B is

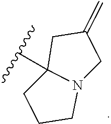 [SMILES: *C12CCCN1CC(=C)C2]

In one embodiment, ring B is

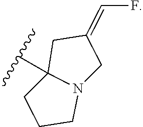 [SMILES: *C12CCCN1C/C(=C\F)C2]

In one embodiment, ring B is

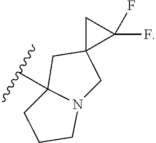 [SMILES: *C12CCCN1CC1(C2)CC1(F)F]

In one embodiment, ring B is

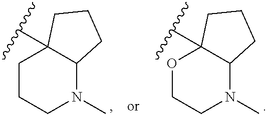 [SMILES: *C12CCCC1N(C)CCC2 ; *C12CCCC1N(C)CCO2]

In one embodiment, ring B is

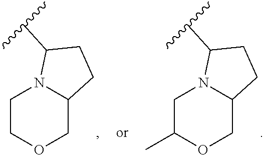 [SMILES: *C1CCC2COCCN12 ; *C1CCC2COC(C)CN12]

In one embodiment, ring B is

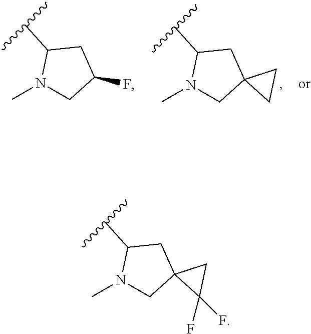

In one embodiment, ring B is

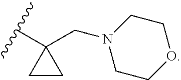 [SMILES: *C1(CN2CCOCC2)CC1]

In one embodiment, ring B is

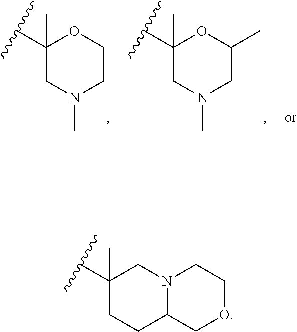

In one embodiment, ring B is

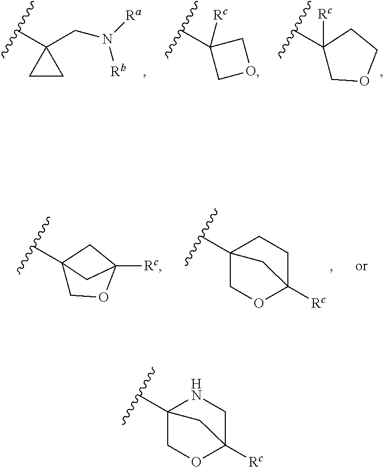

wherein each of R^{a} and R^{b} is, independently, substituted or unsubstituted C₁₋₄ alkyl; substituted or unsubstituted C₃₋₅ cycloalkyl; or R^{a} and R^{b} together with the N to which they are attached form a substituted or unsubstituted heterocycle containing N, O or S; and
R^{c} is H, halogen, CN, substituted or unsubstituted C₁₋₄ alkyl, substituted or unsubstituted C₃₋₅ cycloalkyl, substituted or unsubstituted C₁₋₄ alkoxyl. In one embodiment, ring B is

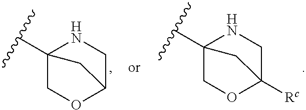

In one embodiment, the compound is

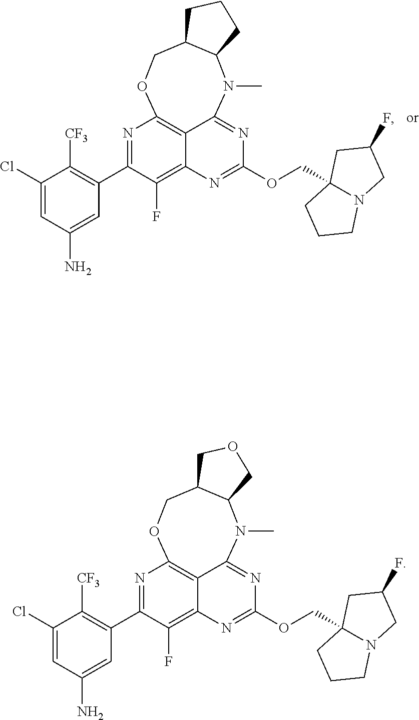 [SMILES: CN1c2nc(OC[C@@]34CCCN3C[C@H](F)C4)nc3c(F)c(-c4cc(N)cc(Cl)c4C(F)(F)F)nc(c23)OC[C@H]2CCC[C@H]21 ; CN1c2nc(OC[C@@]34CCCN3C[C@H](F)C4)nc3c(F)c(-c4cc(N)cc(Cl)c4C(F)(F)F)nc(c23)OC[C@H]2COC[C@H]21]

In one embodiment, the compound is

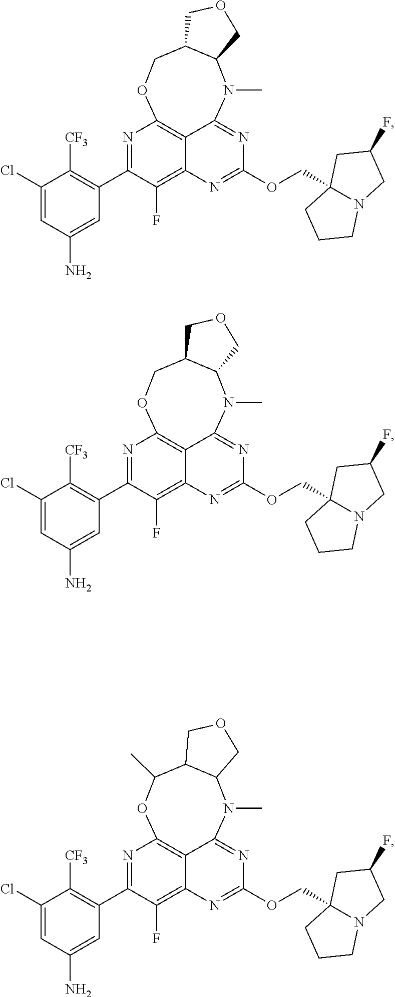 [SMILES: CN1c2nc(OC[C@@]34CCCN3C[C@H](F)C4)nc3c(F)c(-c4cc(N)cc(Cl)c4C(F)(F)F)nc(c23)OC[C@@H]2COC[C@H]21 ; CN1c2nc(OC[C@@]34CCCN3C[C@H](F)C4)nc3c(F)c(-c4cc(N)cc(Cl)c4C(F)(F)F)nc(c23)OC[C@H]2COC[C@@H]21 ; CC1Oc2nc(-c3cc(N)cc(Cl)c3C(F)(F)F)c(F)c3nc(OC[C@@]45CCCN4C[C@H](F)C5)nc(c23)N(C)C2COCC12]

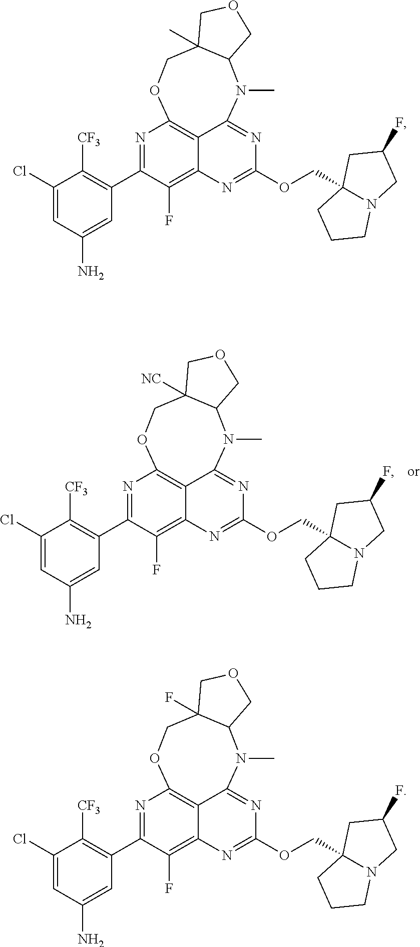 [SMILES: CN1c2nc(OC[C@@]34CCCN3C[C@H](F)C4)nc3c(F)c(-c4cc(N)cc(Cl)c4C(F)(F)F)nc(c23)OCC2(C)COCC12 ; CN1c2nc(OC[C@@]34CCCN3C[C@H](F)C4)nc3c(F)c(-c4cc(N)cc(Cl)c4C(F)(F)F)nc(c23)OCC2(C#N)COCC12 ; CN1c2nc(OC[C@@]34CCCN3C[C@H](F)C4)nc3c(F)c(-c4cc(N)cc(Cl)c4C(F)(F)F)nc(c23)OCC2(F)COCC12]

In one embodiment, the compound is

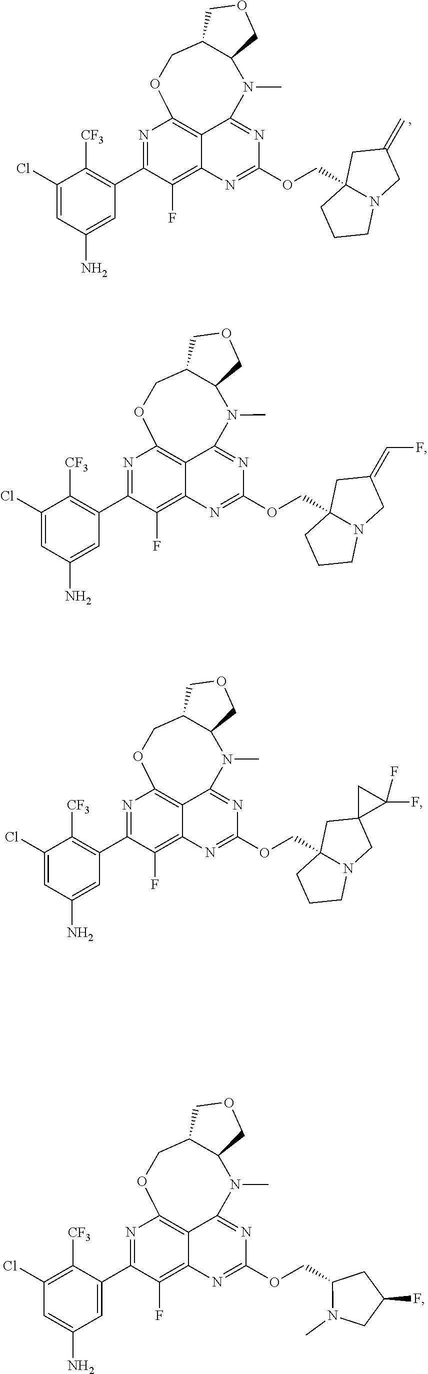 [SMILES: C=C1CN2CCC[C@@]2(COc2nc3c4c(nc(-c5cc(N)cc(Cl)c5C(F)(F)F)c(F)c4n2)OC[C@@H]2COC[C@H]2N3C)C1 ; CN1c2nc(OC[C@@]34CCCN3C/C(=C\F)C4)nc3c(F)c(-c4cc(N)cc(Cl)c4C(F)(F)F)nc(c23)OC[C@@H]2COC[C@H]21 ; CN1c2nc(OC[C@@]34CCCN3CC3(CC3(F)F)C4)nc3c(F)c(-c4cc(N)cc(Cl)c4C(F)(F)F)nc(c23)OC[C@@H]2COC[C@H]21 ; CN1C[C@H](F)C[C@H]1COc1nc2c3c(nc(-c4cc(N)cc(Cl)c4C(F)(F)F)c(F)c3n1)OC[C@@H]1COC[C@H]1N2C]

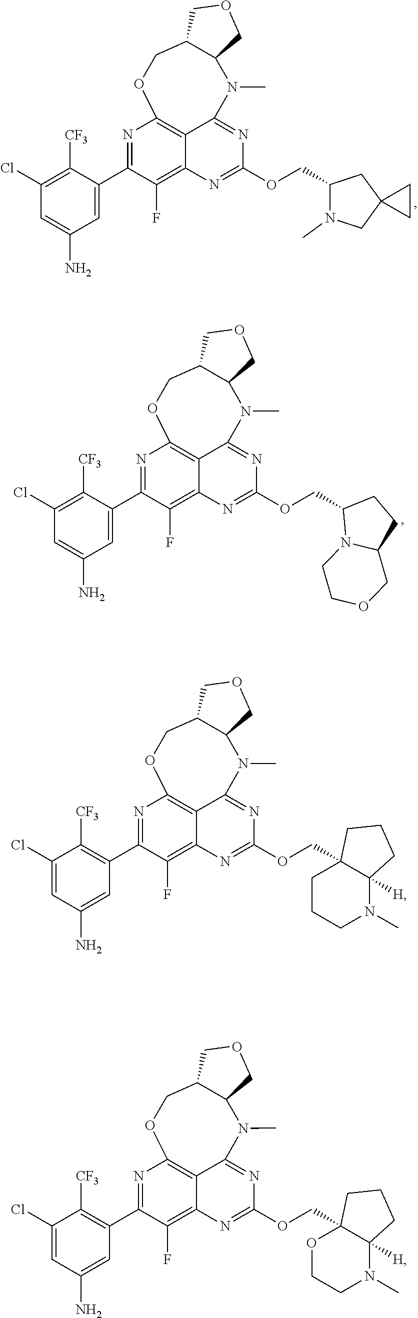

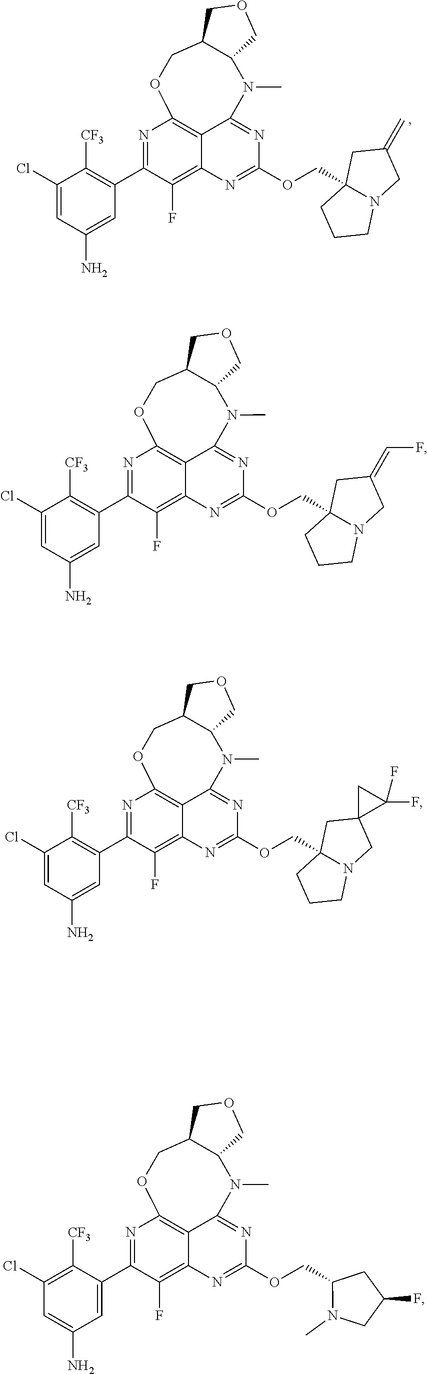 [SMILES: C=C1CN2CCC[C@@]2(COc2nc3c4c(nc(-c5cc(N)cc(Cl)c5C(F)(F)F)c(F)c4n2)OC[C@H]2COC[C@@H]2N3C)C1 ; CN1c2nc(OC[C@@]34CCCN3C/C(=C\F)C4)nc3c(F)c(-c4cc(N)cc(Cl)c4C(F)(F)F)nc(c23)OC[C@H]2COC[C@@H]21 ; CN1c2nc(OC[C@@]34CCCN3CC3(CC3(F)F)C4)nc3c(F)c(-c4cc(N)cc(Cl)c4C(F)(F)F)nc(c23)OC[C@H]2COC[C@@H]21 ; CN1C[C@H](F)C[C@H]1COc1nc2c3c(nc(-c4cc(N)cc(Cl)c4C(F)(F)F)c(F)c3n1)OC[C@H]1COC[C@@H]1N2C]

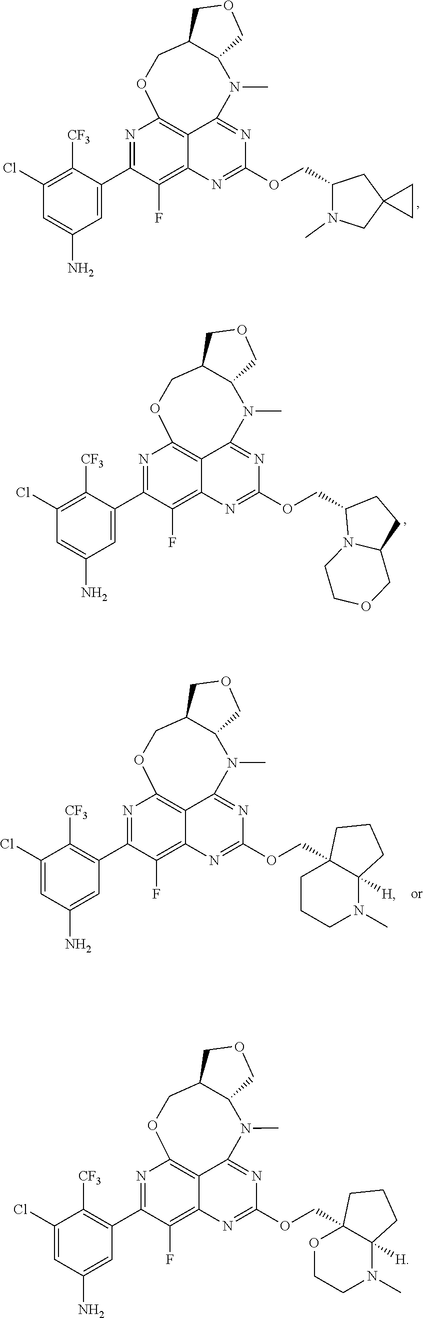

Aspect 19: Provided herein is a compound selected from the following table:

TABLE 3

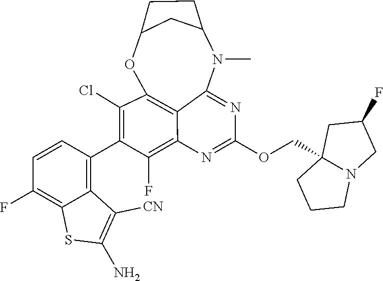 [SMILES: CN1c2nc(OC[C@@]34CCCN3C[C@H](F)C4)nc3c(F)c(-c4ccc(F)c5sc(N)c(C#N)c45)c(Cl)c(c23)OC2CCC1C2]

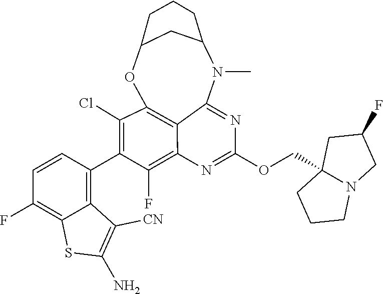 [SMILES: CN1c2nc(OC[C@@]34CCCN3C[C@H](F)C4)nc3c(F)c(-c4ccc(F)c5sc(N)c(C#N)c45)c(Cl)c(c23)OC2CCCC1C2]

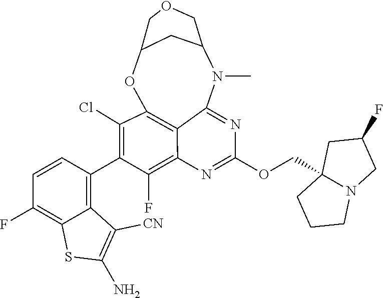 [SMILES: CN1c2nc(OC[C@@]34CCCN3C[C@H](F)C4)nc3c(F)c(-c4ccc(F)c5sc(N)c(C#N)c45)c(Cl)c(c23)OC2COCC1C2]

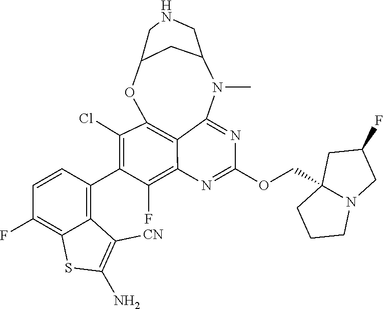 [SMILES: CN1c2nc(OC[C@@]34CCCN3C[C@H](F)C4)nc3c(F)c(-c4ccc(F)c5sc(N)c(C#N)c45)c(Cl)c(c23)OC2CNCC1C2]

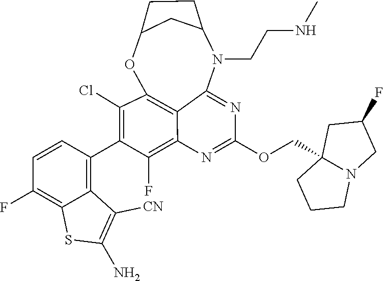 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CCC1C2]

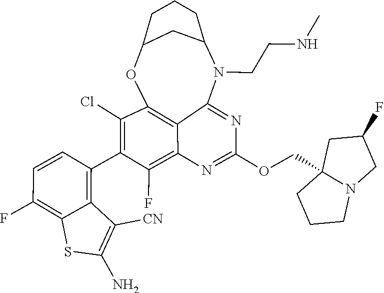 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2CCCC1C2]

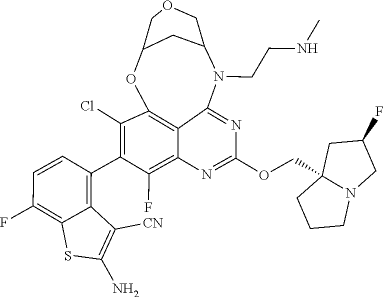 [SMILES: CNCCN1c2nc(OC[C@@]34CCCN3C[C@H](F)C4)nc3c(F)c(-c4ccc(F)c5sc(N)c(C#N)c45)c(Cl)c(c23)OC2COCC1C2]

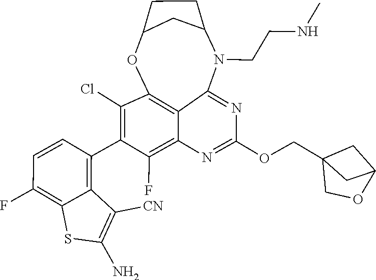 [SMILES: CNCCN1c2nc(OCC34COC(C3)C4)nc3c(F)c(-c4ccc(F)c5sc(N)c(C#N)c45)c(Cl)c(c23)OC2CCC1C2]

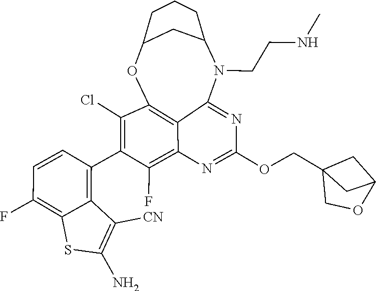 [SMILES: CNCCN1c2nc(OCC34COC(C3)C4)nc3c(F)c(-c4ccc(F)c5sc(N)c(C#N)c45)c(Cl)c(c23)OC2CCCC1C2]

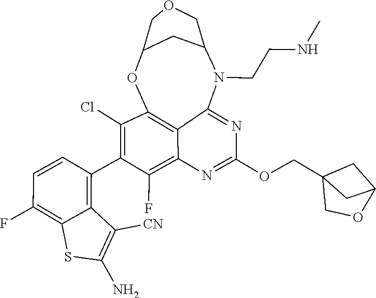 [SMILES: CNCCN1c2nc(OCC34COC(C3)C4)nc3c(F)c(-c4ccc(F)c5sc(N)c(C#N)c45)c(Cl)c(c23)OC2COCC1C2]

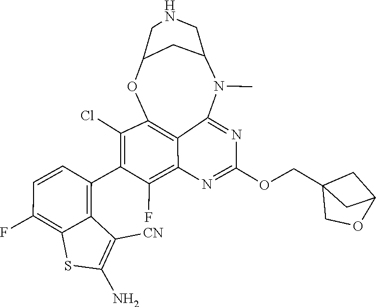 [SMILES: CN1c2nc(OCC34COC(C3)C4)nc3c(F)c(-c4ccc(F)c5sc(N)c(C#N)c45)c(Cl)c(c23)OC2CNCC1C2]

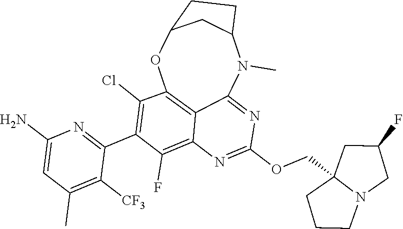 [SMILES: Cc1cc(N)nc(-c2c(Cl)c3c4c(nc(OC[C@@]56CCCN5C[C@H](F)C6)nc4c2F)N(C)C2CCC(C2)O3)c1C(F)(F)F]

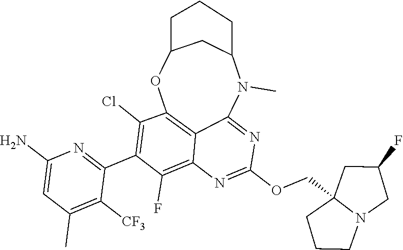 [SMILES: Cc1cc(N)nc(-c2c(Cl)c3c4c(nc(OC[C@@]56CCCN5C[C@H](F)C6)nc4c2F)N(C)C2CCCC(C2)O3)c1C(F)(F)F]

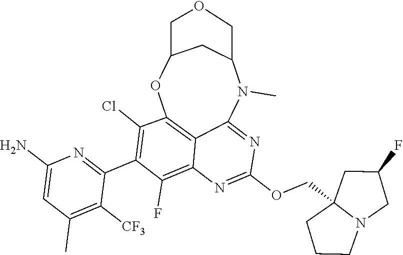 [SMILES: Cc1cc(N)nc(-c2c(Cl)c3c4c(nc(OC[C@@]56CCCN5C[C@H](F)C6)nc4c2F)N(C)C2COCC(C2)O3)c1C(F)(F)F]

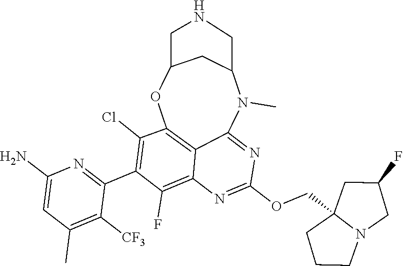 [SMILES: Cc1cc(N)nc(-c2c(Cl)c3c4c(nc(OC[C@@]56CCCN5C[C@H](F)C6)nc4c2F)N(C)C2CNCC(C2)O3)c1C(F)(F)F]

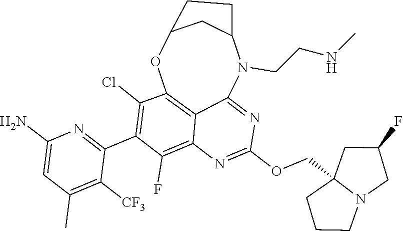 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CCC1C2]

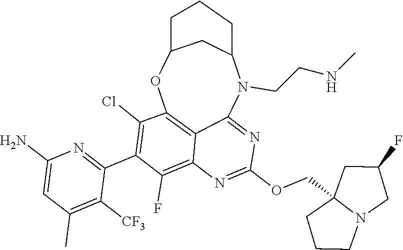 [SMILES: CNCCN1c2nc(OC[C@@]34CCCN3C[C@H](F)C4)nc3c(F)c(-c4nc(N)cc(C)c4C(F)(F)F)c(Cl)c(c23)OC2CCCC1C2]

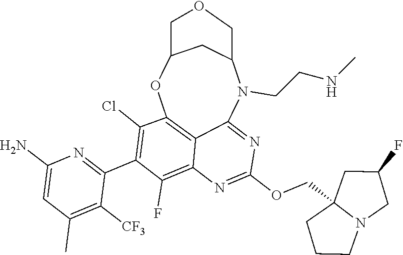 [SMILES: CNCCN1c2nc(OC[C@@]34CCCN3C[C@H](F)C4)nc3c(F)c(-c4nc(N)cc(C)c4C(F)(F)F)c(Cl)c(c23)OC2COCC1C2]

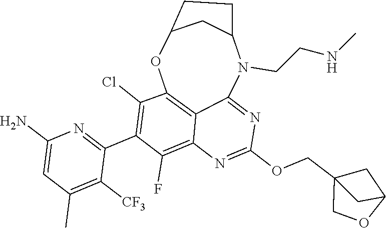 [SMILES: CNCCN1c2nc(OCC34COC(C3)C4)nc3c(F)c(-c4nc(N)cc(C)c4C(F)(F)F)c(Cl)c(c23)OC2CCC1C2]

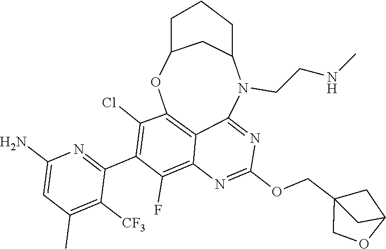 [SMILES: CNCCN1c2nc(OCC34COC(C3)C4)nc3c(F)c(-c4nc(N)cc(C)c4C(F)(F)F)c(Cl)c(c23)OC2CCCC1C2]

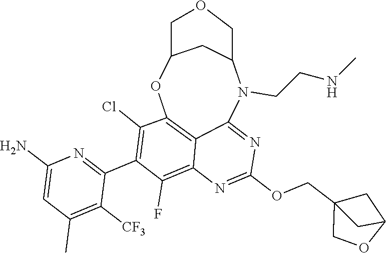 [SMILES: CNCCN1c2nc(OCC34COC(C3)C4)nc3c(F)c(-c4nc(N)cc(C)c4C(F)(F)F)c(Cl)c(c23)OC2COCC1C2]

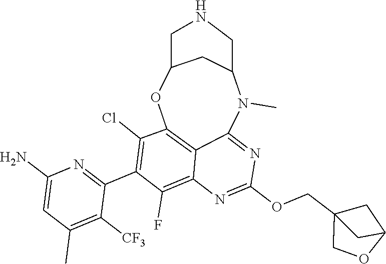 [SMILES: Cc1cc(N)nc(-c2c(Cl)c3c4c(nc(OCC56COC(C5)C6)nc4c2F)N(C)C2CNCC(C2)O3)c1C(F)(F)F]

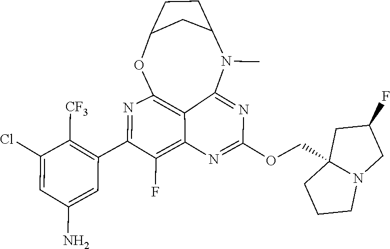 [SMILES: CN1c2nc(OC[C@@]34CCCN3C[C@H](F)C4)nc3c(F)c(-c4cc(N)cc(Cl)c4C(F)(F)F)nc(c23)OC2CCC1C2]

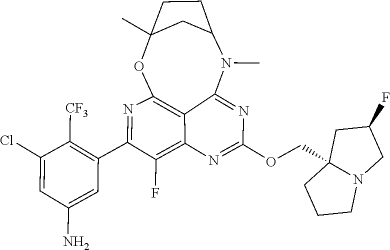 [SMILES: CN1c2nc(OC[C@@]34CCCN3C[C@H](F)C4)nc3c(F)c(-c4cc(N)cc(Cl)c4C(F)(F)F)nc(c23)OC2(C)CCC1C2]

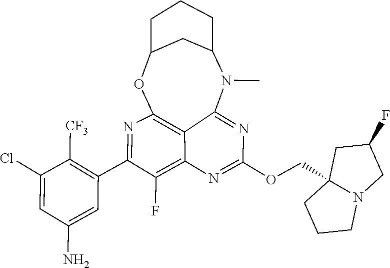 [SMILES: CN1c2nc(OC[C@@]34CCCN3C[C@H](F)C4)nc3c(F)c(-c4cc(N)cc(Cl)c4C(F)(F)F)nc(c23)OC2CCCC1C2]

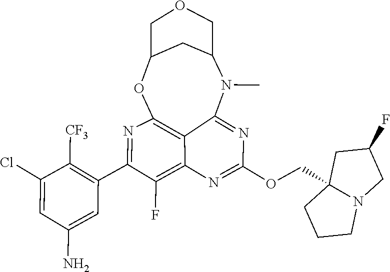 [SMILES: CN1c2nc(OC[C@@]34CCCN3C[C@H](F)C4)nc3c(F)c(-c4cc(N)cc(Cl)c4C(F)(F)F)nc(c23)OC2COCC1C2]

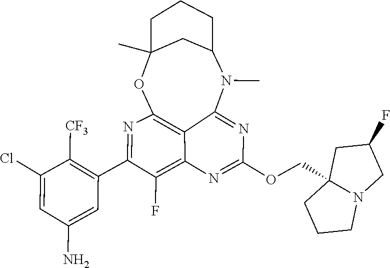 [SMILES: CN1c2nc(OC[C@@]34CCCN3C[C@H](F)C4)nc3c(F)c(-c4cc(N)cc(Cl)c4C(F)(F)F)nc(c23)OC2(C)CCCC1C2]

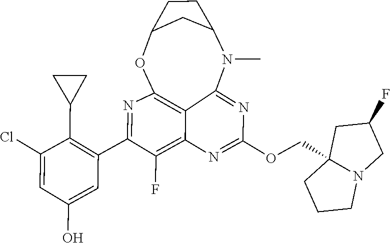 [SMILES: CN1c2nc(OC[C@@]34CCCN3C[C@H](F)C4)nc3c(F)c(-c4cc(O)cc(Cl)c4C4CC4)nc(c23)OC2CCC1C2]

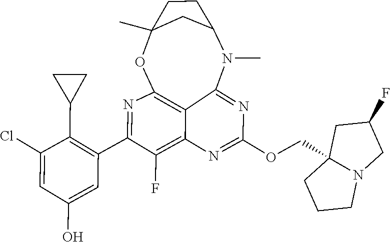 [SMILES: CN1c2nc(OC[C@@]34CCCN3C[C@H](F)C4)nc3c(F)c(-c4cc(O)cc(Cl)c4C4CC4)nc(c23)OC2(C)CCC1C2]

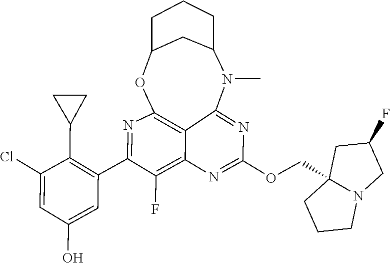 [SMILES: CN1c2nc(OC[C@@]34CCCN3C[C@H](F)C4)nc3c(F)c(-c4cc(O)cc(Cl)c4C4CC4)nc(c23)OC2CCCC1C2]

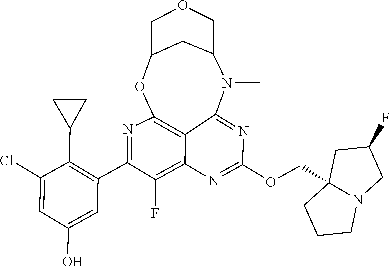 [SMILES: CN1c2nc(OC[C@@]34CCCN3C[C@H](F)C4)nc3c(F)c(-c4cc(O)cc(Cl)c4C4CC4)nc(c23)OC2COCC1C2]

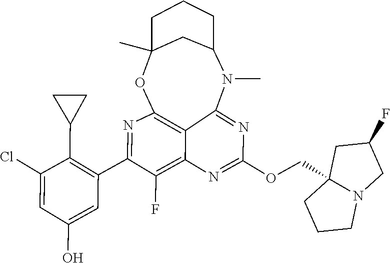 [SMILES: CN1c2nc(OC[C@@]34CCCN3C[C@H](F)C4)nc3c(F)c(-c4cc(O)cc(Cl)c4C4CC4)nc(c23)OC2(C)CCCC1C2]

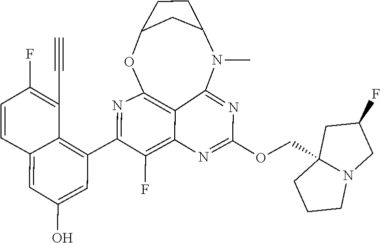 [SMILES: C#Cc1c(F)ccc2cc(O)cc(-c3nc4c5c(nc(OC[C@@]67CCCN6C[C@H](F)C7)nc5c3F)N(C)C3CCC(C3)O4)c12]

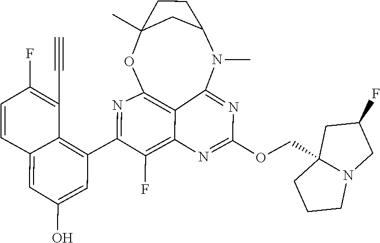 [SMILES: C#Cc1c(F)ccc2cc(O)cc(-c3nc4c5c(nc(OC[C@@]67CCCN6C[C@H](F)C7)nc5c3F)N(C)C3CCC(C)(C3)O4)c12]

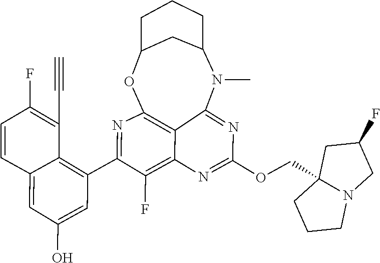 [SMILES: C#Cc1c(F)ccc2cc(O)cc(-c3nc4c5c(nc(OC[C@@]67CCCN6C[C@H](F)C7)nc5c3F)N(C)C3CCCC(C3)O4)c12]

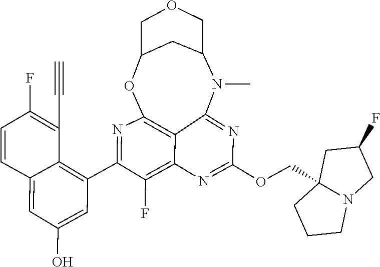 [SMILES: C#Cc1c(F)ccc2cc(O)cc(-c3nc4c5c(nc(OC[C@@]67CCCN6C[C@H](F)C7)nc5c3F)N(C)C3COCC(C3)O4)c12]

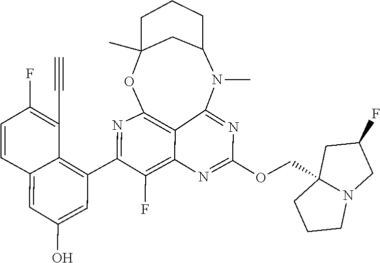 [SMILES: C#Cc1c(F)ccc2cc(O)cc(-c3nc4c5c(nc(OC[C@@]67CCCN6C[C@H](F)C7)nc5c3F)N(C)C3CCCC(C)(C3)O4)c12]

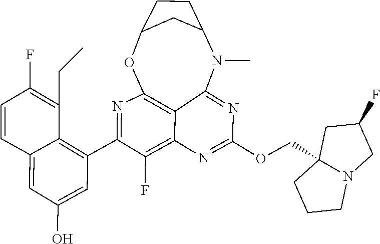 [SMILES: CCc1c(F)ccc2cc(O)cc(-c3nc4c5c(nc(OC[C@@]67CCCN6C[C@H](F)C7)nc5c3F)N(C)C3CCC(C3)O4)c12]

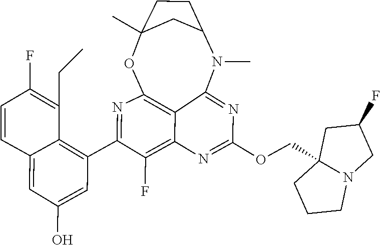 [SMILES: CCc1c(F)ccc2cc(O)cc(-c3nc4c5c(nc(OC[C@@]67CCCN6C[C@H](F)C7)nc5c3F)N(C)C3CCC(C)(C3)O4)c12]

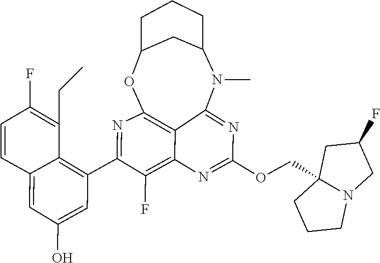 [SMILES: CCc1c(F)ccc2cc(O)cc(-c3nc4c5c(nc(OC[C@@]67CCCN6C[C@H](F)C7)nc5c3F)N(C)C3CCCC(C3)O4)c12]

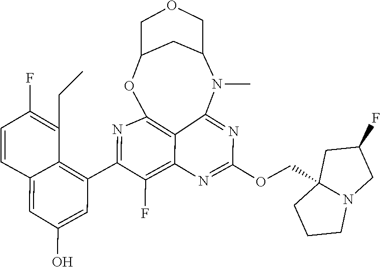 [SMILES: CCc1c(F)ccc2cc(O)cc(-c3nc4c5c(nc(OC[C@@]67CCCN6C[C@H](F)C7)nc5c3F)N(C)C3COCC(C3)O4)c12]

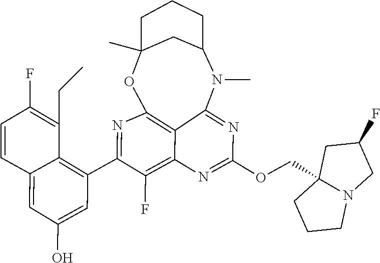 [SMILES: CCc1c(F)ccc2cc(O)cc(-c3nc4c5c(nc(OC[C@@]67CCCN6C[C@H](F)C7)nc5c3F)N(C)C3CCCC(C)(C3)O4)c12]

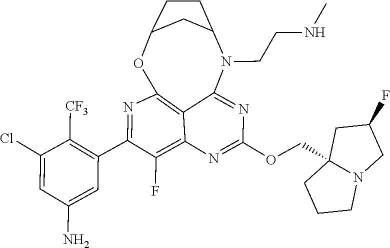 [SMILES: CNCCN1c2nc(OC[C@@]34CCCN3C[C@H](F)C4)nc3c(F)c(-c4cc(N)cc(Cl)c4C(F)(F)F)nc(c23)OC2CCC1C2]

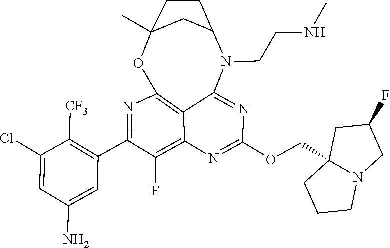 [SMILES: CNCCN1c2nc(OC[C@@]34CCCN3C[C@H](F)C4)nc3c(F)c(-c4cc(N)cc(Cl)c4C(F)(F)F)nc(c23)OC2(C)CCC1C2]

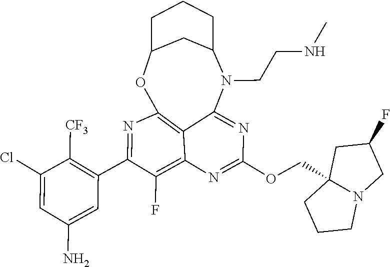 [SMILES: CNCCN1c2nc(OC[C@@]34CCCN3C[C@H](F)C4)nc3c(F)c(-c4cc(N)cc(Cl)c4C(F)(F)F)nc(c23)OC2CCCC1C2]

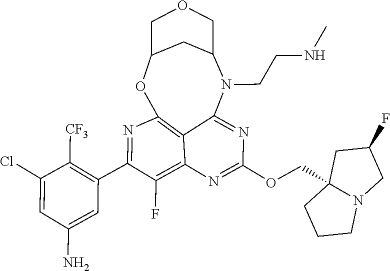 [SMILES: CNCCN1c2nc(OC[C@@]34CCCN3C[C@H](F)C4)nc3c(F)c(-c4cc(N)cc(Cl)c4C(F)(F)F)nc(c23)OC2COCC1C2]

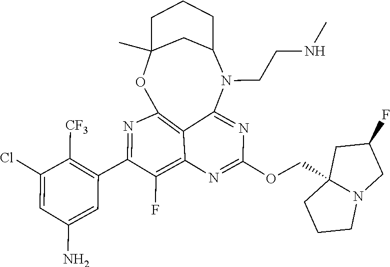 [SMILES: CNCCN1c2nc(OC[C@@]34CCCN3C[C@H](F)C4)nc3c(F)c(-c4cc(N)cc(Cl)c4C(F)(F)F)nc(c23)OC2(C)CCCC1C2]

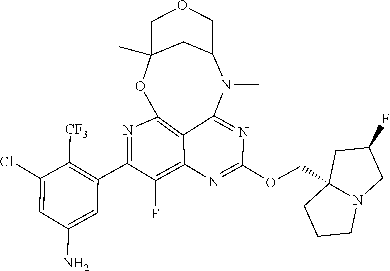 [SMILES: CN1c2nc(OC[C@@]34CCCN3C[C@H](F)C4)nc3c(F)c(-c4cc(N)cc(Cl)c4C(F)(F)F)nc(c23)OC2(C)COCC1C2]

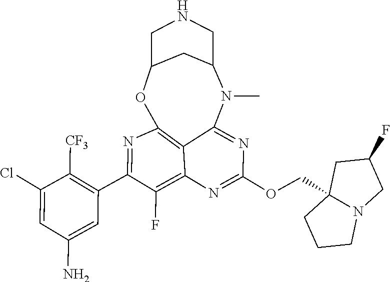 [SMILES: CN1c2nc(OC[C@@]34CCCN3C[C@H](F)C4)nc3c(F)c(-c4cc(N)cc(Cl)c4C(F)(F)F)nc(c23)OC2CNCC1C2]

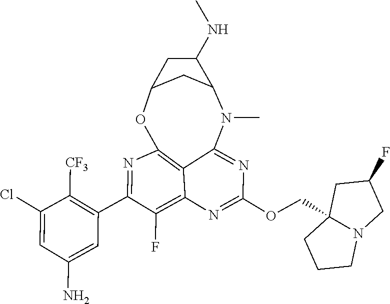 [SMILES: CNC1CC2CC1N(C)c1nc(OC[C@@]34CCCN3C[C@H](F)C4)nc3c(F)c(-c4cc(N)cc(Cl)c4C(F)(F)F)nc(c13)O2]

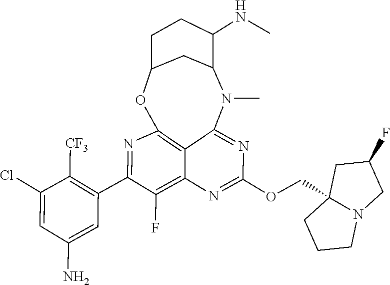 [SMILES: CNC1CCC2CC1N(C)c1nc(OC[C@@]34CCCN3C[C@H](F)C4)nc3c(F)c(-c4cc(N)cc(Cl)c4C(F)(F)F)nc(c13)O2]

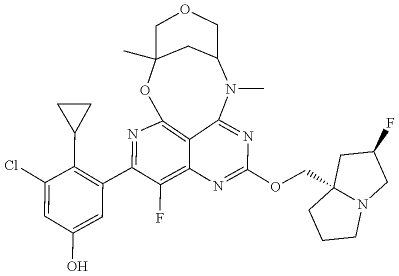 [SMILES: CN1c2nc(OC[C@@]34CCCN3C[C@H](F)C4)nc3c(F)c(-c4cc(O)cc(Cl)c4C4CC4)nc(c23)OC2(C)COCC1C2]

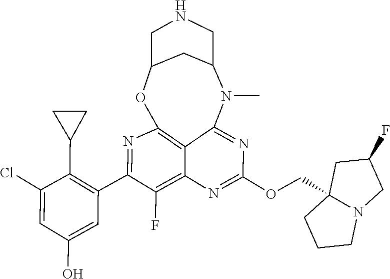 [SMILES: CN1c2nc(OC[C@@]34CCCN3C[C@H](F)C4)nc3c(F)c(-c4cc(O)cc(Cl)c4C4CC4)nc(c23)OC2CNCC1C2]

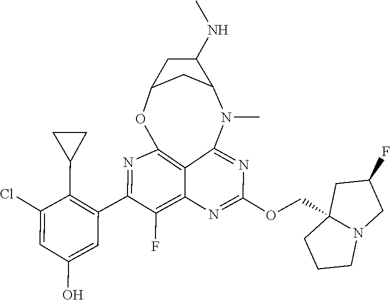 [SMILES: CNC1CC2CC1N(C)c1nc(OC[C@@]34CCCN3C[C@H](F)C4)nc3c(F)c(-c4cc(O)cc(Cl)c4C4CC4)nc(c13)O2]

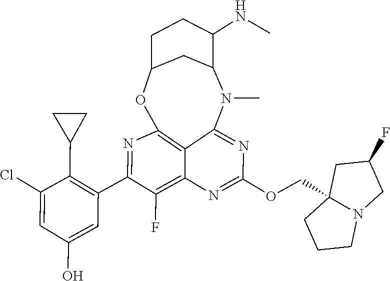 [SMILES: CNC1CCC2CC1N(C)c1nc(OC[C@@]34CCCN3C[C@H](F)C4)nc3c(F)c(-c4cc(O)cc(Cl)c4C4CC4)nc(c13)O2]

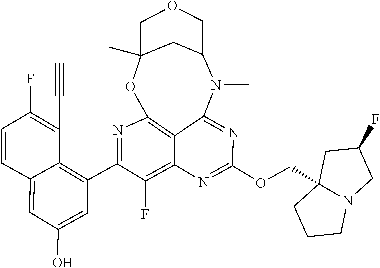 [SMILES: C#Cc1c(F)ccc2cc(O)cc(-c3nc4c5c(nc(OC[C@@]67CCCN6C[C@H](F)C7)nc5c3F)N(C)C3COCC(C)(C3)O4)c12]

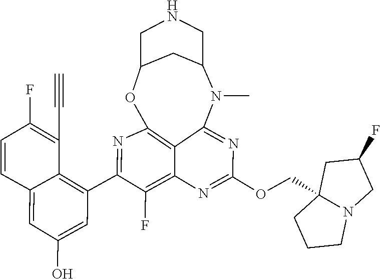 [SMILES: C#Cc1c(F)ccc2cc(O)cc(-c3nc4c5c(nc(OC[C@@]67CCCN6C[C@H](F)C7)nc5c3F)N(C)C3CNCC(C3)O4)c12]

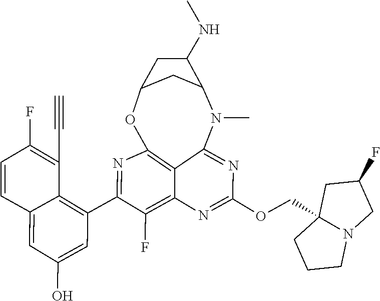 [SMILES: C#Cc1c(F)ccc2cc(O)cc(-c3nc4c5c(nc(OC[C@@]67CCCN6C[C@H](F)C7)nc5c3F)N(C)C3CC(CC3NC)O4)c12]

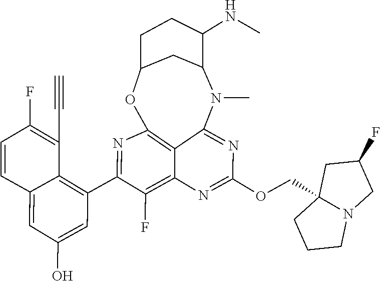 [SMILES: C#Cc1c(F)ccc2cc(O)cc(-c3nc4c5c(nc(OC[C@@]67CCCN6C[C@H](F)C7)nc5c3F)N(C)C3CC(CCC3NC)O4)c12]

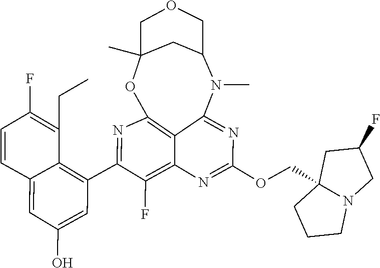 [SMILES: CCc1c(F)ccc2cc(O)cc(-c3nc4c5c(nc(OC[C@@]67CCCN6C[C@H](F)C7)nc5c3F)N(C)C3COCC(C)(C3)O4)c12]

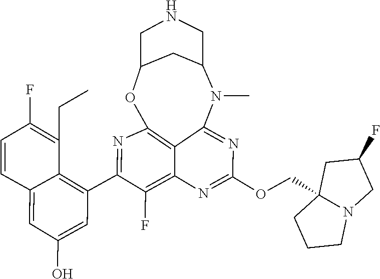 [SMILES: CCc1c(F)ccc2cc(O)cc(-c3nc4c5c(nc(OC[C@@]67CCCN6C[C@H](F)C7)nc5c3F)N(C)C3CNCC(C3)O4)c12]

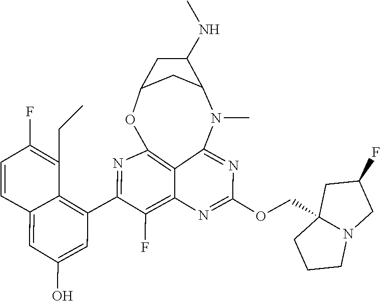 [SMILES: CCc1c(F)ccc2cc(O)cc(-c3nc4c5c(nc(OC[C@@]67CCCN6C[C@H](F)C7)nc5c3F)N(C)C3CC(CC3NC)O4)c12]

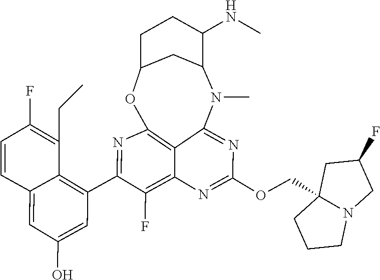 [SMILES: CCc1c(F)ccc2cc(O)cc(-c3nc4c5c(nc(OC[C@@]67CCCN6C[C@H](F)C7)nc5c3F)N(C)C3CC(CCC3NC)O4)c12]

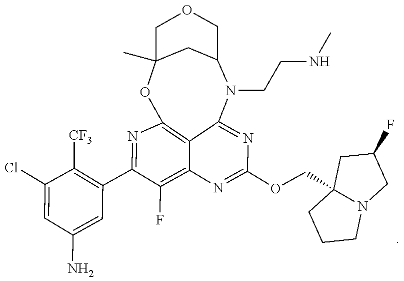 [SMILES: CNCCN1c2nc(OC[C@@]34CCCN3C[C@H](F)C4)nc3c(F)c(-c4cc(N)cc(Cl)c4C(F)(F)F)nc(c23)OC2(C)COCC1C2]

Aspect 20: In one embodiment, provided herein is a pharmaceutical composition comprising an effective amount of a compound provided herein, or a pharmaceutically acceptable salt, tautomer, isotopologue, stereoisomer, enantiomer, atropisomer, or prodrug thereof, and a pharmaceutically acceptable carrier, excipient or vehicle.

Aspect 21: In one embodiment, provided herein is a method for inhibiting the activity of KRAS mutant protein in a cell, comprising contacting said cell with an effective amount of a compound provided herein, or a pharmaceutically acceptable salt, tautomer, isotopologue, stereoisomer, enantiomer, atropisomer, or prodrug thereof, optionally wherein the KRAS mutant protein is KRAS G12D and/or G12V mutant protein. In one embodiment, provided herein is a method for inhibiting the activity of KRAS amplification in a cell, comprising contacting said cell with an effective amount of a compound provided herein, or a pharmaceutically acceptable salt, tautomer, isotopologue, stereoisomer, enantiomer, atropisomer, or prodrug thereof, optionally wherein the KRAS mutant protein is KRAS G12D and/or G12V mutant protein.

Aspect 22: In one embodiment, provided herein is a method for treatment or prevention of cancer, the method comprising administering to a subject in need thereof an effective amount of a compound provided herein, or a pharmaceutically acceptable salt, tautomer, isotopologue, stereoisomer, enantiomer, atropisomer, or prodrug thereof, optionally wherein the cancer is mediated by KRAS mutation; preferably KRAS G12D and/or G12V mutation. Provided here is a method for the treatment or prevention of a cancer, the methods comprising administering to a subject in need thereof an effective amount of a compound provided herein.

Aspect 23: Provided here is a method of modulating activity of KRAS G12D and/or G12V, comprising contacting said cell with an effective amount of a compound provided herein, or a pharmaceutically acceptable salt, tautomer, isotopologue, stereoisomer, enantiomer, atropisomer, or prodrug thereof.

Aspect 24: Provided herein is a kit for treating cancer, the kit comprising (a) a pharmaceutical composition comprising a compound provided herein; and (b) instructions for administration of an effective amount of the pharmaceutical composition comprising the KRAS G12D and/or G12V inhibitor provided herein to treat cancer in an individual.

The present embodiments can be understood more fully by reference to the detailed description and examples, which are intended to exemplify non-limiting embodiments.

### Methods for Making Compounds

The Compounds can be made using conventional organic syntheses and commercially available starting materials. By way of example and not limitation, Compounds of formula (I) can be prepared as outlined in Schemes 1-3 shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Examplary protecting groups are described in “Protective Groups in Organic Synthesis”, 4^{th} Edition, P. G. M. Wuts; T. W. Greene, John Wiley, 2007, and references cited therein.

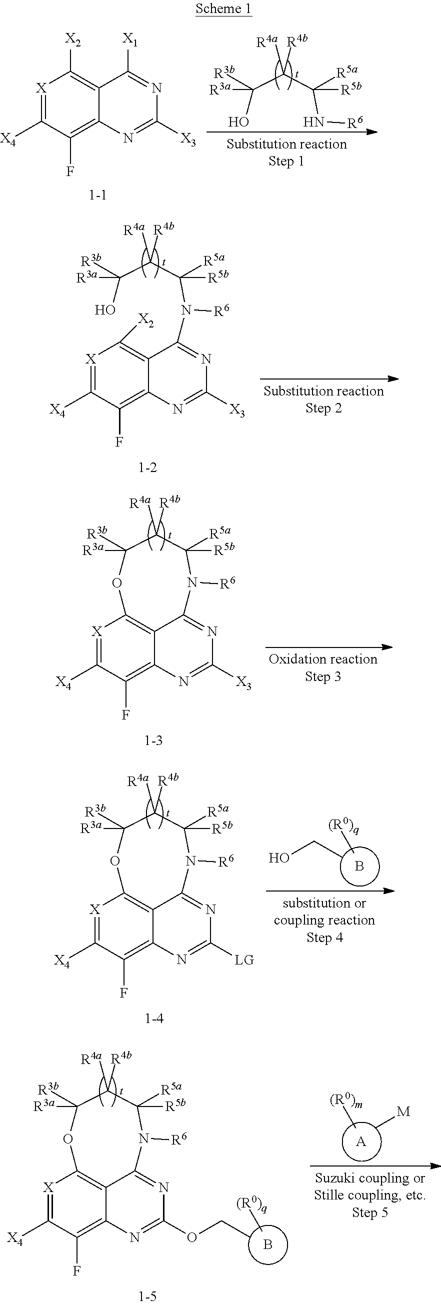

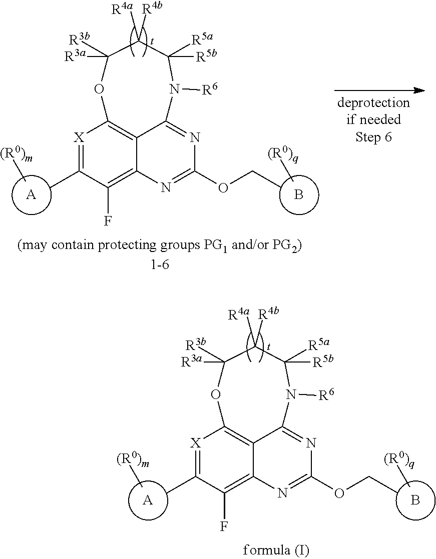

As shown in Scheme 1, in some embodiments, provided herein are methods for preparing the compounds defined as formula (I). Halogen substituted compound 1-1 (X₂ and X₄ are halogen, X₁ is OH or Cl, and X₃ could be methylthiolyl) is converted into compound 1-2 under substitution conditions (e.g., HATU, DIEA, if X₁ is OH; DIEA, DCM is X₁ is Cl); then compound 1-2 is converted to compound 1-3 under substitution conditions (e.g., NaH, THF); then compound 1-3 is converted to compound 1-4 under oxidation conditions (m-CPBA oxidation if LG is methyl sulfonyl or methyl sulfinyl); then compound 1-4 is converted to compound 1-5 followed by substitution or coupling reactions (e.g., NaH, THF); compound 1-5 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 1-6, wherein M can be boronic acid, boronic ester, a metal (such as Zn), tributyltin, etc.; finally, protecting groups containing compound 1-6 is then deprotected (e.g., TFA and DCM to deprotect Boc group when PG₁ and PG₂ contains Boc group, CsF and DMF to deprotect TIPS group when PG, and PG₂ contains TIPS group) to yield the compound defined as formula (I).

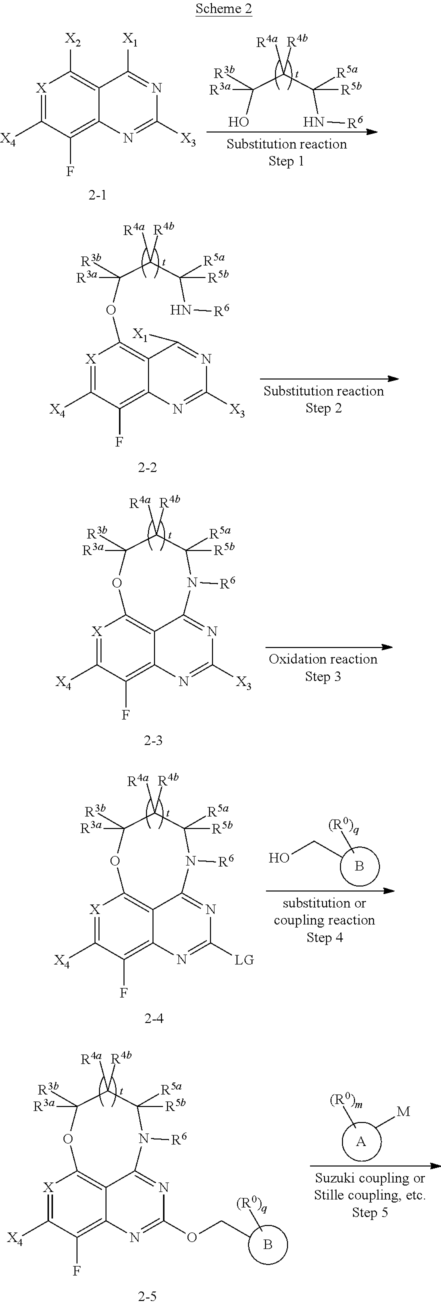

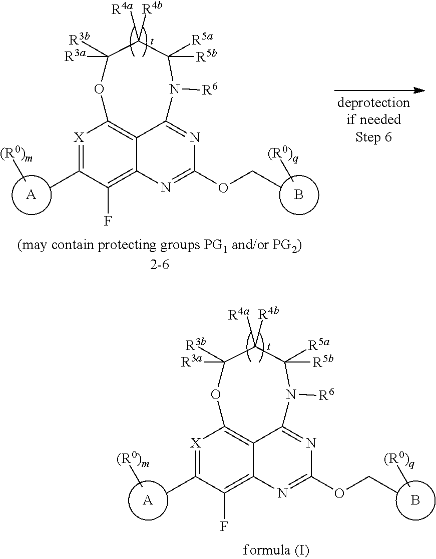

As shown in Scheme 2, in some embodiments, provided herein are methods for preparing the compounds defined as formula (I). Halogen substituted compound 2-1 (X₂ and X₄ are halogen, X, is OH or Cl, and X₃ could be methylthiolyl) is converted into compound 2-2 under substitution conditions (e.g., NaH, THF); then compound 2-2 is converted to compound 2-3 under substitution conditions (e.g., HATU, DIEA, if X₁ is OH; DIEA, DCM is X₁ is Cl); then compound 2-3 is converted to compound 2-4 under oxidation conditions (m-CPBA oxidation if LG is methyl sulfonyl or methyl sulfinyl); then compound 2-4 is converted to compound 2-5 followed by substitution or coupling reactions (e.g., NaH, THF); compound 2-5 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 2-6, wherein M can be boronic acid, boronic ester, a metal (such as Zn), tributyltin, etc.; finally, protecting groups containing compound 2-6 is then deprotected (e.g., TFA and DCM to deprotect Boc group when PG₁ and PG₂ contains Boc group, CsF and DMF to deprotect TIPS group when PG₁ and PG₂ contains TIPS group) to yield the compound defined as formula (I).

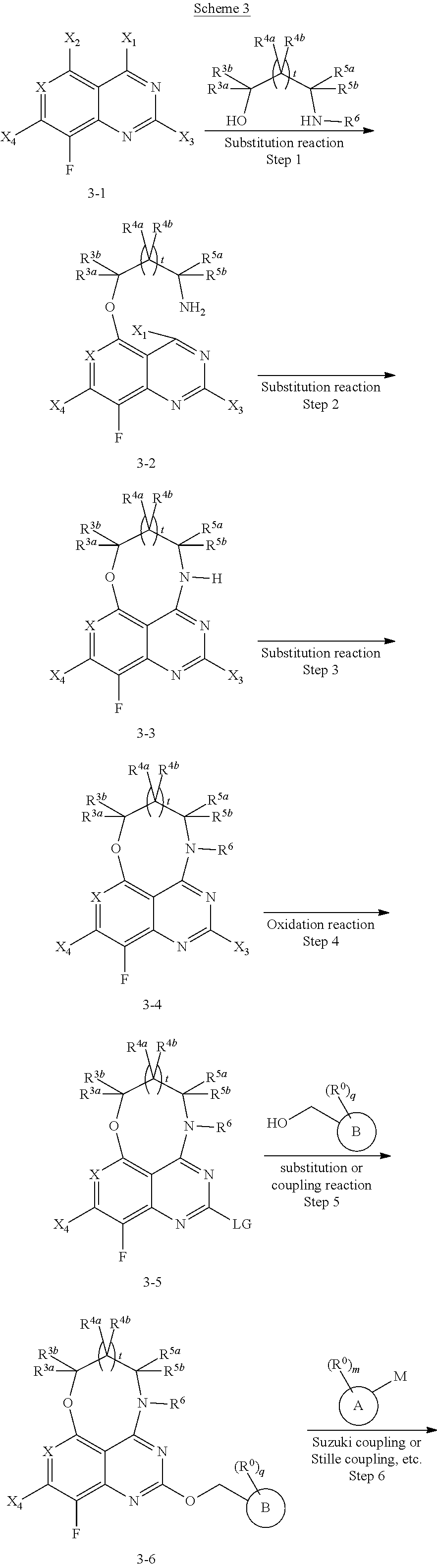

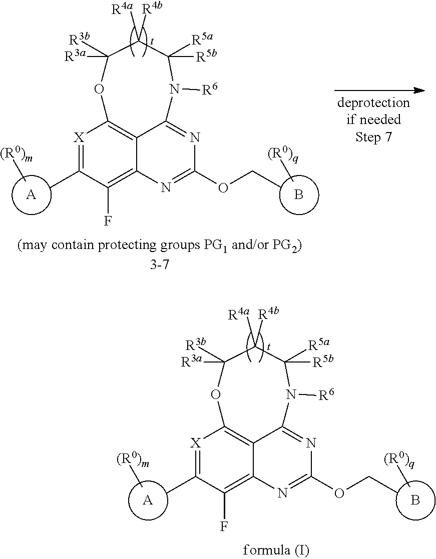

As shown in Scheme 3, in some embodiments, provided herein are methods for preparing the compounds defined as formula (I). Halogen substituted compound 3-1 (X₂ and X₄ are halogen, X₁ is OH or Cl, and X₃ could be methylthiolyl) is converted into compound 3-2 under substitution conditions (e.g., NaH, THF); then compound 3-2 is converted to compound 3-3 under substitution conditions (e.g., HATU or BOPCl, DIEA, if X₁ is OH; DIEA, DCM is X₁ is Cl); then compound 3-3 is converted to compound 3-4 under substitution conditions (e.g. NaH and Mel, if R⁶ is Me); then compound 3-4 is converted to compound 3-5 under oxidation conditions (m-CPBA oxidation if LG is methyl sulfonyl or methyl sulfinyl); then compound 3-5 is converted to compound 3-6 followed by substitution or coupling reactions (e.g., NaH, THF); compound 3-6 further undergoes metal catalyzed cross-coupling reaction such as Suzuki, Negishi, or Stille coupling (e.g. Pd(dtbpf)Cl₂, K₃PO₄, 1,4-dioxane, water for Suzuki coupling) to obtain compound 3-7, wherein M can be boronic acid, boronic ester, a metal (such as Zn), tributyltin, etc.; finally, protecting groups containing compound 3-7 is then deprotected (e.g., TFA and DCM to deprotect Boc group when PG₁ and PG; contains Boc group, CsF and DMF to deprotect TIPS group when PG₁ and PG₂ contains TIPS group) to yield the compound defined as formula (I).

The present embodiments can be understood more fully by reference to the detailed description and examples, which are intended to exemplify non-limiting embodiments.

### Examples

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz or 500 MHz with TMS (tetramethylsilane) as the internal standard. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), m (quintuplet), sx (sextuplet), m (multiplet), br (broadened), dd (doublet of doublets), dt (doublet of triplets). Coupling constants, when given, are reported in Hertz (Hz).

LC/MS data was recorded by using Agilent 1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 19.1.

In the following examples, the following abbreviations are used:
AcOH Acetic acid
Aq. Aqueous
BINAP 2,2′-bis(diphenylphosphino)-1,1′-binaphthalene
Brine Saturated aqueous sodium chloride solution
Bn Benzyl
BnBr Benzyl Bromide
Boc Tert-butoxycarbonyl
BOP Benzotriazol-1-yl-oxy-tris-(dimethylamino)phosphonium hexa-fluorophosphate
BOPCl bis(2-oxo-3-oxazolidinyl)phosphinic chloride
CH₂Cl₂ or DCM Dichloromethane
CAN Cerium(IV) ammonium nitrate (cericammonium nitrate)
Cs₂CO₃ Cesium carbonate
DAST Diethylaminosulfur trifluoride
DCM Dichloromethane
DMF N,N-Dimethylformamide
Dppf 1,1′-bis(diphenylphosphino)ferrocene
DBU 1,8-diazabicyclo[5.4.0]undec-7-ene
DHP 3,4-Dihydro-2H-pyran
DIEA or DIPEA N,N-diisopropylethylamine
DMAP 4-N,N-dimethylaminopyridine
DMB (2,4-dimethoxyphenyl)methanamine
Dess-Martin/DMP Dess-Martin Periodinane
DMF N,N-dimethylformamide
DMF-DMA N,N-Dimethylformamide dimethyl acetal purum
DMSO Dimethyl sulfoxide
DMEDA Dimethyl Ethylene Diamine
EDCI 1-Ethyl-3-(3-dimethylaminopropyl)carbodiimide hydrochloride
EtOAc or EA Ethyl acetate
EtOH Ethanol
Et₃SiH Triethyl silhydride
Et₂O or ether Diethyl ether
g Grams
h or hr Hour
HATU O-(7-Azabenzotriazol-1-yl)-N,N,N′,N′-tetramethyluronium hexafluorophosphate
Hex Hexane
HCl Hydrochloric acid
HMDS Hexamethyldisilazane
HOBT 1-Hydroxybenzotriazole
HPLC High-performance liquid chromatography
IBX 2-Iodylbenzoic acid
i-PrOH Isopropyl alcohol
LCMS Liquid chromatography-mass spectrometry
LDA Lithium diisopropylamide
LiHMDS Lithium Bis(trimethylsilyl)amide
K₂OsO₄·H₂O Potassium osmate(VI) dihydrate
K₃PO₄ Tripotassium phosphate
mg Milligrams
mL Milliliters
mmol Millimole
MeCN Acetonitrile
MeOH Methanol
Min Minutes
ms or MS Mass spectrum
m-CPBA 2-chloranylbenzenecarboperoxoic acid
MOM Methoxymethyl
MPLC Medium Pressure Liquid Chromatography
Na₂SO₄ Sodium sulfate
NaBH(OAc)₃/STAB Sodium triacetyl borohydride
NaH Sodium hydride
NaHMDS Sodium bis(trimethylsilyl)amide
NBS N-Bromosuccinimide
NCS N-Chlorosuccinimide
NMO 4-Methylmorpholine N-oxide
NMP N-Methyl Pyrrolidone
PE petroleum ether
PMB (4-methoxyphenyl)methanamine
POCl₃ phosphorous oxychloride
PyBOP Benzotriazol-1-yl-oxytripyrrolidinophosphonium hexafluorophosphate
PddppfCl2 [1,1′-Bis(diphenylphosphino)ferrocene]dichloropalladium(II)
Pd(dtbpf)Cl₂ [1,1′-Bis(di-tert-butylphosphino)ferrocene]dichloropalladium(II) Tris(dibenzylideneacetone)dipalladium
Pd₂(dba)₃
Prep Preparative
PTSA 4-Methylbenzenesulfonic acid
Rt or rt Room temperature
RuPhos 2-Dicyclohexylphosphino-2′,6′-diisopropoxybiphenyl
sat. Saturated
SEMCl (2-(Chloromethoxy)ethyl)trimethylsilane
TBSCl tert-Butyldimethylsilyl chloride
TEA/Et3N triethylamine
t-BuOK Potassium tert-butoxide
t-BuONa Sodium tert-butoxide
T₃P n-Propylphosphonic cyclic anhydride
TIPS Triisopropylsilyl
Trimethylsilyl cyanide TMSCN
TFA Trifluoroacetic acid
TFAA Trifluoroacetic anhydride
THF Tetrahydrofuran
TLC thin layer chromatography
tBuXPhospd-G3 Methanesulfonato(2-di-t-butylphosphino-2′,4′,6′-tri-i-propyl-1,1′-biphenyl)(2′-amino-1,1′-biphenyl-2-yl)palladium(II)
tBuXPhos 2-Di-tert-butylphosphino-2′,4′,6′-triisopropylbiphenyl
UHP Urea hydrogen peroxide
μL Microliters
XantPhos 4,5-Bis(diphenylphosphino)-9,9-dimethylxanthene
XPhos 2-Dicyclohexylphosphino-2′,4′,6′-triisopropylbiphenyl
4 CzIPN (4r, 6r)-2,4,5,6-tetra(9H-carbazol-9-yl)isophthalonitrile

### Compound Synthesis

Example 1: 5-ethynyl-6-fluoro-4-(11-fluoro-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazolin-10-yl)naphthalen-2-ol

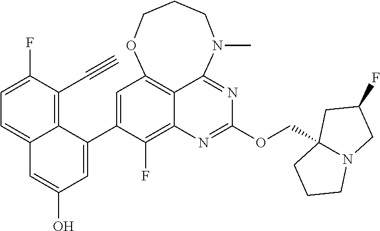 [SMILES: C#Cc1c(F)ccc2cc(O)cc(-c3cc4c5c(nc(OC[C@@]67CCCN6C[C@H](F)C7)nc5c3F)N(C)CCCO4)c12]

Step 1: 7-bromo-4-chloro-5,8-difluoro-2-(methylthio)quinazoline

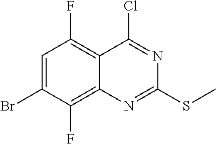 [SMILES: CSc1nc(Cl)c2c(F)cc(Br)c(F)c2n1]

To a suspension of 7-bromo-5,8-difluoro-2-(methylthio)quinazolin-4(3H)-one (10 g, 32.6 mmol) in POCl₃ (30 mL), DIPEA (15 mL) was added dropwise. The mixture was stirred at 100° C. overnight. Then, the mixture was cooled to rt, and concentrated in vacuo. The mixture was diluted with DCM, filtered off the solid to give crude product, and further purified by silica gel column chromatography (80 g, eluting with PE/EtOAc=100%: 0%˜ 75%: 25%) to give the title compound (6.9 g). MS (ESI, m/e) [M+H]⁺ 324.4.

Step 2: 3-((7-bromo-5,8-difluoro-2-(methylthio)quinazolin-4-yl)(methyl)amino)propan-1-ol

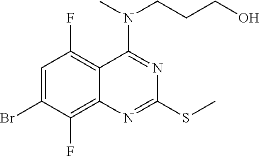 [SMILES: CSc1nc(N(C)CCCO)c2c(F)cc(Br)c(F)c2n1]

To a solution of 7-bromo-4-chloro-5,8-difluoro-2-(methylthio)quinazoline (1.2 g, 3.7 mmol) and 3-(methylamino)propan-1-ol (0.4 g, 4.6 mmol) in DCM (20 mL) was added DIEA (1.43 g, 11,1 mol) at 0° C. The mixture was stirred at room temperature for 1 h. Then, the mixture was concentrated in vacuo, and the residue was purified by silica gel column chromatography (12 g, eluting with PE/EtOAc=100%: 0%˜ 0%: 100%) to give the title compound (540 mg). MS (ESI, m/e) [M+H]⁺ 378.0.

Step 3: 10-bromo-11-fluoro-4-methyl-2-(methylthio)-4,5,6,7-tetrahydro-[1,5]oxazocino[4,3,2-de]quinazoline

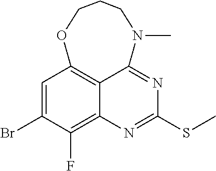 [SMILES: CSc1nc2c3c(cc(Br)c(F)c3n1)OCCCN2C]

To a solution of 3-((7-bromo-5,8-difluoro-2-(methylthio)quinazolin-4-yl)(methyl)amino)propan-1-ol (540 mg, 1.4 mmol) in THF (20 mL) was added NaH (171 mg, 4.3 mmol, 60%) in portions at 0° C., and the mixture was stirred at 55° C. overnight. The mixture was cooled to room temperature, quenched with ice-water (2.5 mL), extracted with EtOAc (20 mL*3). The combined organic layer was concentrated in vacuo, and the residue was purified by silica gel column chromatography (12 g, eluting with PE/EtOAc=100%: 0%˜ 0%: 100%) to give the title compound (190 mg). MS (ESI, m/e) [M+H]⁺ 358.1.

Step 4: 10-bromo-11-fluoro-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazoline

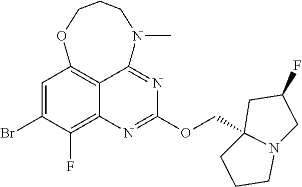 [SMILES: CN1CCCOc2cc(Br)c(F)c3nc(OC[C@@]45CCCN4C[C@H](F)C5)nc1c23]

To a mixture of 10-bromo-11-fluoro-4-methyl-2-(methylthio)-4,5,6,7-tetrahydro-[1,5]oxazocino[4,3,2-de]quinazoline (190 mg, 0.5 mmol) in DCM (10 mL) was added m-CPBA (104 mg, 0.6 mmol) in portions at 0° C. The mixture was stirred at 0° C. for 2 hrs. In the meantime, to a mixture of ((2R,7aS)-2-fluorotetrahydro-1H-pyrrolizin-7a(5H)-yl)methanol (253 mg, 1.59 mmol) in THF (5 mL) was added LiHMDS (1.06 mL, 1.06 mmol, 1M in THF) dropwise at 0° C. The mixture was stirred at rt for another 30 mins, and the resulting THE mixture was added into above DCM solution dropwise. The mixture was stirred at rt for 2 hrs, quenched with MeOH (5 mL), and the mixture was concentrated in vacuo. The residue was purified with chromatography column on silica (eluting with DCM/MeOH=90%: 10%) to give the title compound (48 mg). MS (ESI, m/e) [M+H]⁺ 469.3.

Step 5: 11-fluoro-10-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazoline

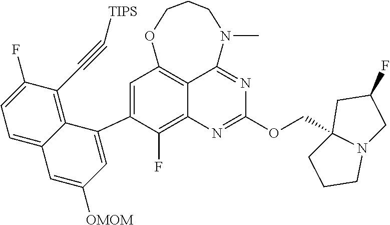

A mixture of 10-bromo-11-fluoro-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazoline (48 mg, 0.1 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (105 mg, 0.2 mmol), 1,1′-Bis(diphenylphosphino)ferrocene-palladium(II)dichloride dichloromethane complex (8.5 mg, 0.01 mmol), NaHCO₃ (17.3 mg, 0.2 mmol) in dioxane/water (10 mL/2 mL) was stirred at 90° C. overnight. The mixture was cooled to room temperature, concentrated under vacuo, and the residue was purified with chromatography column (4g, eluting with DCM/MeOH=90%: 10%) to give the crude product, which was further purified by prep-TLC (DCM/MeOH=17/1 twice) to give the title compound (10 mg). MS (ESI, m/e) [M+H]⁺ 775.7.

Step 6: 10-(8-ethynyl-7-fluoro-3-(methoxymethoxy)naphthalen-1-yl)-11-fluoro-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazoline

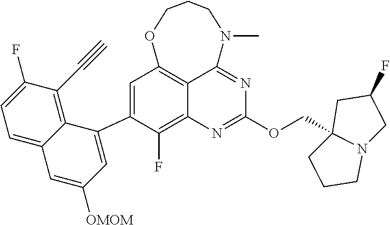

To a solution of 11-fluoro-10-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazoline (10 mg, 0.01 mmol) in DMF (5 mL) was added CsF (10 mg, 0.06 mmol). The mixture was stirred at rt for 2 hrs. Then, the mixture was filtered, the filtration was concentrated in vacuo to give the crude product, which was used in next step without further purification (13 mg, crude). MS (ESI, m/e) [M+H]⁺ 619.5.

Step 7: 5-ethynyl-6-fluoro-4-(11-fluoro-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazolin-10-yl)naphthalen-2-ol

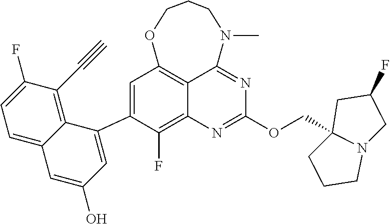 [SMILES: C#Cc1c(F)ccc2cc(O)cc(-c3cc4c5c(nc(OC[C@@]67CCCN6C[C@H](F)C7)nc5c3F)N(C)CCCO4)c12]

To a solution of 10-(8-ethynyl-7-fluoro-3-(methoxymethoxy)naphthalen-1-yl)-11-fluoro-2-(((2R,7aS)-2-fluorotetrahydro-1H-pyrrolizin-7a(5H)-yl)methoxy)-4-methyl-4,5,6,7-tetrahydro-[1,5]oxazocino[4,3,2-de]quinazoline (13 mg, 0.021 mmol) in dioxane (4 mL) was added HCl (2 mL, 4M in dioxane). The mixture was stirred at rt for 2 hrs. The mixture was concentrated via vacuo, and purified with prep-HPLC to give the title product (4.2 mg). ¹H NMR (500 MHZ, DMSO-d₆) δ 10.10 (s, 1H), 7.99-7.90 (m, 1H), 7.50-7.39 (m, 1H), 7.37-7.29 (m, 1H), 7.07 (s, 1H), 6.70-6.62 (m, 1H), 5.36-5.20 (m, 1H), 4.32-4.17 (m, 2H), 4.12-4.06 (m, 1H), 4.00-3.92 (m, 2H), 3.64-3.50 (m, 2H), 3.27 (s, 3H), 3.14-3.06 (m, 2H), 3.02 (s, 1H), 2.85-2.83 (m. 1H), 2.17-2.07 (m, 1H), 2.05-1.93 (m, 4H), 1.89-1.71 (m, 3H). MS (ESI, m/c) [M+H]⁺ 575.2.

Example 2: 2-amino-7-fluoro-4-(11-fluoro-2-(((2R,7aS)-2-fluorotetrahydro-1H-pyrrolizin-7a(5H)-yl)methoxy)-4-methyl-4.5,6,7-tetrahydro-[1,5]oxazocino[4,3,2-de]quinazolin-10-yl)benzo[b]thiophene-3-carbonitrile

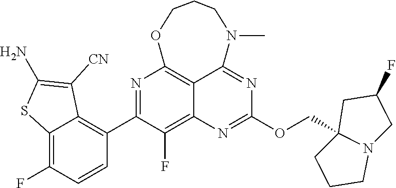 [SMILES: CN1CCCOc2nc(-c3ccc(F)c4sc(N)c(C#N)c34)c(F)c3nc(OC[C@@]45CCCN4C[C@H](F)C5)nc1c23]

Example 2 was prepared by a procedure similar to that described in Example 1 (step 5/6/7) by replacing ((2-fluoro-6-(methoxymethoxy)-8-(4,4,5,5-tetramethyl-1,3,2-dioxaborolan-2-yl)naphthalen-1-yl)ethynyl)triisopropylsilane with tert-butyl (3-cyano-7-fluoro-4-(4,4,5,5-tetramethyl-1,3,2-dioxaborolan-2-yl)benzo[b]thiophen-2-yl)carbamate to give the title product (0.84 mg). ¹H NMR (500 MHz, DMSO-d₆) δ 8.06-7.99 (s, 2H), 7.31-7.21 (m, 1H), 7.10-7.06 (m, 1H), 6.71-6.60 (m, 1H), 5.36-5.20 (m, 1H), 4.30-4.00 (m, 4H), 3.59-3.54 (s, 2H), 3.20-3.00 (m, 2H), 2.89-2.82 (m, 2H), 2,25-1.76 (m, 7H). MS (ESI, m/e) [M+H]⁺ 581.4.

Example 3: 3-(4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-methyl-4-(trifluoromethyl)aniline

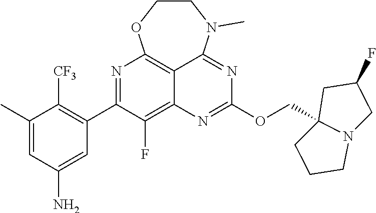 [SMILES: Cc1cc(N)cc(-c2nc3c4c(nc(OC[C@@]56CCCN5C[C@H](F)C6)nc4c2F)N(C)CCO3)c1C(F)(F)F]

Step 1: 2-((5,7-dichloro-8-fluoro-2-(methylthio)pyrido[4,3-d]pyrimidin-4-yl)(methyl)amino)ethan-1-ol

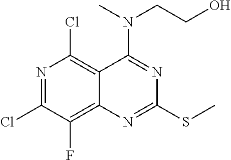 [SMILES: CSc1nc(N(C)CCO)c2c(Cl)nc(Cl)c(F)c2n1]

To a solution of 4,5,7-trichloro-8-fluoro-2-(methylthio) pyrido[4,3-d]pyrimidine (296 mg, 1 mmol) in DCM (15 mL) was added DIPEA (387 mg, 3 mmol) and 2-(methylamino) ethan-1-ol (68 mg, 0.9 mmol) at 0° C. The resulting mixture was stirred at 0° C. to room temperature for 1 hour. After completion, the reaction mixture was concentrated to give the residue. The residue was purified by flash column silica (PE: EA=2:1) to give the title product (211 mg). MS (ESI, m/e) [M+H]⁺ 337.1.

Step 2: 5-chloro-4-fluoro-10-methyl-2-(methylthio)-9,10-dihydro-8H-7-oxa-1,3,6,10-tetraazacyclohepta[de]naphthalene

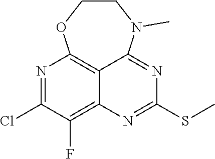 [SMILES: CSc1nc2c3c(nc(Cl)c(F)c3n1)OCCN2C]

To a solution of 2-((5,7-dichloro-8-fluoro-2-(methylthio) pyrido[4,3-d]pyrimidin-4-yl) (methyl)amino) ethan-1-ol (210 mg, 0.625 mmol) in THF (25 mL) was added NaH (50 mg, 1.25 mmol) at room temperature, the mixture was stirred at r.t. for 2 hrs. Upon completion, the mixture was quenched with 2 drops of ice water and the solvent was evaporated. The residue was purified by chromatography column on silica (PE: EA=1:1) to give the title product (40 mg). MS (ESI, m/e) [M+H]⁺ 301,1.

Step 3: 5-chloro-4-fluoro-10-methyl-2-(methylsulfinyl)-9,10-dihydro-8H-7-oxa-1,3,6,10-tetraazacyclohepta[de]naphthalene

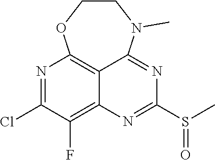 [SMILES: CN1CCOc2nc(Cl)c(F)c3nc(S(C)=O)nc1c23]

To a solution of 5-chloro-4-fluoro-10-methyl-2-(methylthio)-9,10-dihydro-8H-7-oxa-1,3,6,10-tetraazacyclohepta[de]naphthalene (40 mg, 0.13 mmol) in DCM (10 mL) was added m-CPBA (27 mg, 0.16 mmol) at r.t., and the mixture was stirred at r.t. for 1 h. Upon completion, the mixture was diluted with water (10 mL). Aqueous layer was extracted with DCM (20 mL*3), and the combined organic layer was concentrated to give a residue as the title compound (50 mg, crude). MS (ESI, m/e) [M+1]⁺317.1

Step 4: 5-chloro-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalene

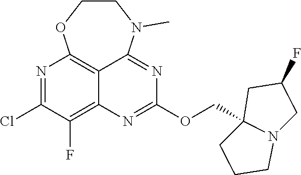 [SMILES: CN1CCOc2nc(Cl)c(F)c3nc(OC[C@@]45CCCN4C[C@H](F)C5)nc1c23]

To a solution of ((2R,7aS)-2-fluorotetrahydro-1H-pyrrolizin-7a(5H)-yl) methanol (75 mg, 0.474 mmol) in 10 mL THF was added LiHMDS (1 N in THF, 0.3 mL, 0.3 mmol) at room temperature and it was stirred at room temperature for 1 hour. Then, a solution of 5-chloro-4-fluoro-10-methyl-2-(methylsulfinyl)-9,10-dihydro-8H-7-oxa-1,3,6,10-tetraazacyclohepta[de]naphthalene (50 mg, 0.158 mmol) in THF (10 mL) was added to the reaction mixture and stirred at 0° C. to room temperature for 1 hour. After completion, solvents were evaporated. The residue was purified by chromatography column on silica (DCM: MeOH=10:1) to give the title product (27 mg, crude). MS (ESI, m/e) [M+H]⁺ 412.1.

Step 5: 3-(4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-methyl-4-(trifluoromethyl)aniline

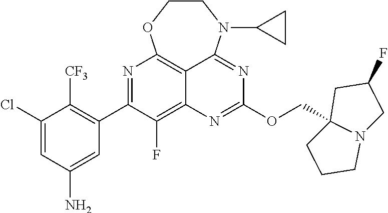 [SMILES: Nc1cc(Cl)c(C(F)(F)F)c(-c2nc3c4c(nc(OC[C@@]56CCCN5C[C@H](F)C6)nc4c2F)N(C2CC2)CCO3)c1]

A mixture of 5-chloro-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalene (36 mg, 0.088 mmol), dioxane/H₂O (10/2 mL), 3-methyl-5-(4,4,5,5-tetramethyl-1,3,2-dioxaborolan-2-yl)-4-(trifluoromethyl)aniline (106 mg, 0.352 mmol), NaHCO₃(22 mg, 0.26 mmol), and Pd(dtbpf)Cl₂ (4 mg, 0.0088 mmol) was stirred at 95° C. for 3 hours. The resulting cooled mixture was concentrated and purified by column chromatography (DCM/MeOH=10/1) to give a residue which was further purified by Prep-HPLC to give the title product (22.7 mg). ¹H NMR (500 MHZ, CD₃OD) δ 6.71-6.65 (m, 1H), 6.44-6.38 (m, 1H), 5.44-5.33 (m, 1H), 4.62-4.54 (m, 2H), 4.43-4.35 (m, 2H), 4.06-3.94 (m, 2H), 3.57-3.36 (m, 6H), 3.19-3.12 (m, 1H), 2.50-1.93 (m, 9H). MS (ESI, m/e) [M+H]⁺ 571.2.

Example 4: 3-chloro-5-(11-cyclopropyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalen-5-yl)-4-(trifluoromethyl)aniline

Step 1: 3-(cyclopropylamino) propan-1-ol

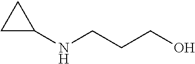

Cyclopropanamine (855 mg, 15 mmol) was mixed with 3-bromopropan-1-ol (417 mg, 3 mmol) at room temperature, and the mixture was stirred at 50° C. overnight. Upon completion, the mixture was concentrated to give a residue as the title compound (658 mg, crude). MS (ESI, m/e) [M+H]⁺ 116.1.

Step 2: 3-(cyclopropyl(5,7-dichloro-8-fluoro-2-(methylthio)pyrido[4,3-d]pyrimidin-4-yl)amino)propan-1-ol

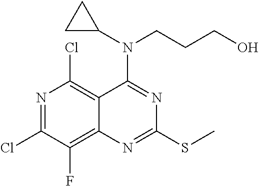 [SMILES: CSc1nc(N(CCCO)C2CC2)c2c(Cl)nc(Cl)c(F)c2n1]

To a solution of 4,5,7-trichloro-8-fluoro-2-(methylthio) pyrido[4,3-d]pyrimidine (498 mg, 1.69 mmol) in DCM (25 mL) was added DIPEA (545 mg, 4.23 mmol) and 3-(cyclopropylamino) propan-1-ol (196 mg, 1.69 mmol) at 0° C. The resulting mixture was stirred at 0° C. to room temperature for 1 hour. After completion, the reaction mixture was concentrated to give the residue. The residue was purified by flash column silica (PE: EA=1:1) to give the title product (230 mg). MS (ESI, m/e) [M+H]⁺ 377.1.

Step 3: 5-chloro-11-cyclopropyl-4-fluoro-2-(methylthio)-8,9,10,11-tetrahydro-7-oxa-1,3,6,11-tetraazacycloocta[de]naphthalene

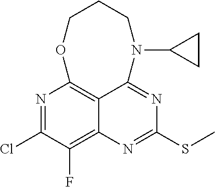 [SMILES: CSc1nc2c3c(nc(Cl)c(F)c3n1)OCCCN2C1CC1]

To a solution of 3-(cyclopropyl(5,7-dichloro-8-fluoro-2-(methylthio)pyrido[4,3-d]pyrimidin-4-yl)amino)propan-1-ol (230 mg, 0.61 mmol) in THF (25 mL) was added NaH (50 mg, 1.22 mmol) at room temperature. The mixture was stirred at r.t. for 2 hrs, then quenched with 2 drops of ice water, and the solvent was evaporated. The residue was purified by chromatography column on silica (PE: EA=3:1) to give the title product (330 mg, crude). MS (ESI, m/e) [M+H]⁺ 341,1.

Step 4: 5-chloro-11-cyclopropyl-4-fluoro-2-(methylsulfinyl)-8,9,10,11-tetrahydro-7-oxa-1,3,6,11-tetraazacycloocta[de]naphthalene

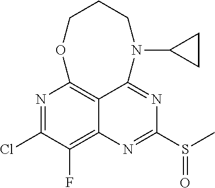 [SMILES: CS(=O)c1nc2c3c(nc(Cl)c(F)c3n1)OCCCN2C1CC1]

To a solution of 5-chloro-11-cyclopropyl-4-fluoro-2-(methylthio)-8,9,10,11-tetrahydro-7-oxa-1,3,6,11-tetraazacycloocta[de]naphthalene (330 mg, 0.62 mmol) in DCM (20 mL) was added m-CPBA (105 mg, 0.62 mmol) at r.t., and the mixture was stirred at r.t. for 1 h. Upon completion, the mixture was diluted with water (10 mL). Aqueous layer was extracted with DCM (20 mL*3), and the combined organic layer was concentrated to give a residue as the title compound (339 mg, crude). MS (ESI, m/e) [M+1]⁺357.1.

Step 5: 5-chloro-11-cyclopropyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalene

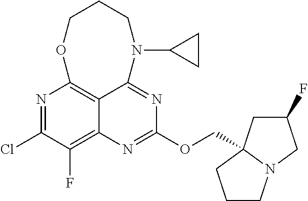 [SMILES: Fc1c(Cl)nc2c3c(nc(OC[C@@]45CCCN4C[C@H](F)C5)nc13)N(C1CC1)CCCO2]

To a solution of ((2R,7aS)-2-fluorotetrahydro-1H-pyrrolizin-7a(5H)-yl) methanol (453 mg, 2.85 mmol) in 20 mL THF was added LiHMDS (1 N in THF, 1.9 mL, 1.9 mmol) at room temperature and it was stirred at room temperature for 1 hour. Then, a solution of 5-chloro-11-cyclopropyl-4-fluoro-2-(methylsulfinyl)-8,9,10,11-tetrahydro-7-oxa-1,3,6,11-tetraazacycloocta[de]naphthalene (339 mg, 0.95 mmol) in THF (10 mL) was added to the reaction mixture and the mixture was stirred at 0° C. to room temperature for 1 hour. After completion, solvent of the reaction mixture was evaporated. The residue was purified by chromatography column on silica (DCM: MeOH=10:1) to give the title product (44 mg, crude). MS (ESI, m/e) [M+H]⁺ 452.1.

Step 6: 3-chloro-5-(11-cyclopropyl-4-fluoro-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalen-5-yl)-4-(trifluoromethyl)aniline

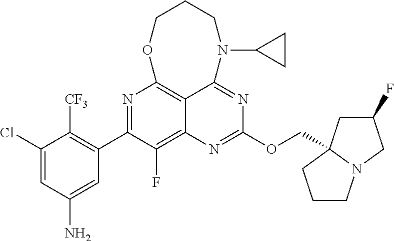 [SMILES: Nc1cc(Cl)c(C(F)(F)F)c(-c2nc3c4c(nc(OC[C@@]56CCCN5C[C@H](F)C6)nc4c2F)N(C2CC2)CCCO3)c1]

To a solution of 5-chloro-11-cyclopropyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalene (22 mg, 0.049 mmol) in dioxane/H₂O (10/2 mL) was added 3-chloro-5-(4,4,5,5-tetramethyl-1,3,2-dioxaborolan-2-yl)-4-(trifluoromethyl)aniline (63 mg, 0.196 mmol), NaHCO₃(12.3 mg, 0.147 mmol) and Pd(dtbpf)Cl₂ (3.2 mg, 0.0049 mmol) at room temperature, and the mixture was stirred at 95° C. for 3 hours. The reaction mixture was cooled to room temperature, concentrated, and the residue was purified by column chromatography (DCM/MeOH=10/1) to give crude product, which was further purified by Prep-HPLC to give the title product (2 mg). ¹H NMR (500 MHZ, CD₃OD) δ6.89-6.85 (m, 1H), 6.55-6.45 (m, 1H), 5.48-5.30 (m, 1H), 4.49-4.39 (m, 4H), 3.75-3.65 (m, 2H), 3.44-3.41 (m, 2H), 3.24-3.13 (m, 3H), 2.48-1.93 (m, 8H), 1.09-0.96 (m, 4H). MS (ESI, m/e) [M+H]⁺ 611.2.

Example 5: 4-(11-cyclopropyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalen-5-yl)-5-ethynyl-6-fluoronaphthalen-2-ol

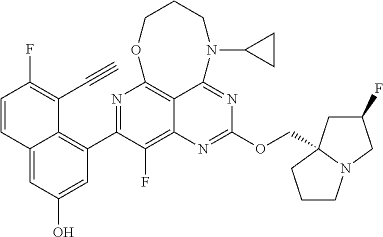 [SMILES: C#Cc1c(F)ccc2cc(O)cc(-c3nc4c5c(nc(OC[C@@]67CCCN6C[C@H](F)C7)nc5c3F)N(C3CC3)CCCO4)c12]

Step 1: 11-cyclopropyl-4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalene

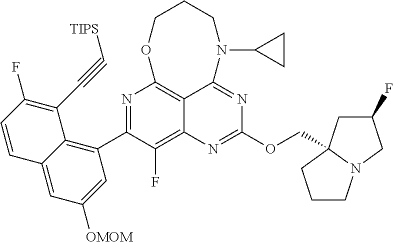

To a solution of 5-chloro-11-cyclopropyl-4-fluoro-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene (22 mg, 0.049 mmol) in dioxane/H2O (10/2 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (100 mg, 0.196 mmol), NaHCO₃(12.3 mg, 0.147 mmol) and Pd(dtbpf)Cl₂ (3.2 mg, 0.0049 mmol) at room temperature, and the mixture was stirred at 95° C. for 3 hours. Upon completion, the mixture was evaporated and purified by chromatography column on silica (DCM: MeOH=20:1) to get the title product (23 mg, crude). MS (ESI, m/e) [M+H]⁺ 802.4.

Step 2: 11-cyclopropyl-5-(8-ethynyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalene

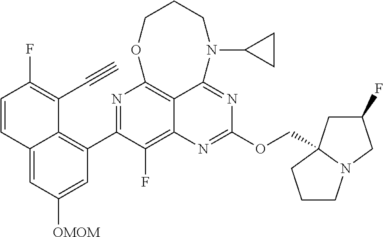

To a solution of 11-cyclopropyl-4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalene (23 mg, 0.029 mmol) in DMF (1.5 mL) was added CsF (22 mg, 0.145 mmol) at room temperature. Upon completion, the mixture was diluted with water (10 mL). Aqueous layer was extracted with DCM (20 mL*3), and the combined organic layer was concentrated to give a residue as the title compound (12 mg, crude). MS (ESI, m/e) [M+H]⁺ 646.3

Step 3: 4-(11-cyclopropyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalen-5-yl)-5-ethynyl-6-fluoronaphthalen-2-ol

To a solution of 11-cyclopropyl-5-(8-ethynyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalene (12 mg, 0.019 mmol) in EtOAc (5 mL) was added HCl (4 N, 1 mL) at room temperature. The resulting mixture was concentrated at room temperature and pH was adjusted to 7 with Na₂CO₃, and the organic layer was concentrated to give a residue which was further purified by Prep-HPLC to give title product (5.6 mg). ¹H NMR (500 MHZ, DMSO-d₆) δ 10.50-9.70 (m, 1H), 7.97-7.94 (m, 1H), 7.47-7.43 (m, 1H), 7.39-7.33 (m, 1H), 7.23-7.17 (m, 1H), 5.34-5.24 (m, 1H), 4.34-4.28 (m, 2H), 4.19-4.17 (m, 1H), 4.12-4.06 (m, 2H), 3.65-3.62 (m, 1H), 3.19-3.03 (m, 3H), 2.88-2.80 (m, 1H), 2.19-1.75 (m, 8H), 1.02-0.83 (m, 4H). MS (ESI, m/e) [M+H]⁺ 602.3.

Example 6: 3-chloro-5-(4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalen-5-yl)-4-(trifluoromethyl)aniline

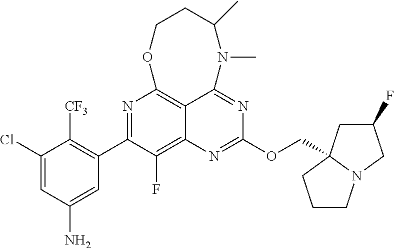 [SMILES: CC1CCOc2nc(-c3cc(N)cc(Cl)c3C(F)(F)F)c(F)c3nc(OC[C@@]45CCCN4C[C@H](F)C5)nc(c23)N1C]

Step 1: 3-(methylamino) butan-1-ol

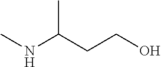

To a solution of tert-butyl (4-hydroxybutan-2-yl) carbamate (1890 mg, 10 mmol) in THF (25 mL) was added LiAlH₄ (650 mg, 17 mmol) at 0° C., and the mixture was stirred at 70° C. overnight. Upon completion, the mixture was cooled to room temperature, sodium sulfate decahydrate was added and stirred for 0.5 h. The resulting mixture was filtered, and the organic phase was concentrated to give a residue as the title compound (707 mg, crude). MS (ESI, m/e) [M+H]⁺ 104.1.

Step 2: 7-chloro-8-fluoro-5-(3-(methylamino)butoxy)-2-(methylthio)pyrido[4,3-d]pyrimidin-4-ol

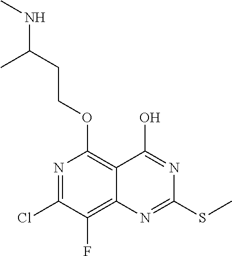 [SMILES: CNC(C)CCOc1nc(Cl)c(F)c2nc(SC)nc(O)c12]

To a solution of 3-(methylamino) butan-1-ol (153 mg, 1.5 mmol) in THF (10 mL) was added sodium hydride (80 mg, 2 mmol) at room temperature. The resulting mixture was stirred at room temperature for 1 hour. Then, 5,7-dichloro-8-fluoro-2-(methylthio) pyrido[4,3-d]pyrimidin-4-ol (279 mg, 1 mmol) in THF (10 mL) was added to the reaction mixture and stirred at room temperature for 1 hour. After completion, the reaction mixture was concentrated to give the residue. The residue was purified by flash column silica (DCM: MeOH=10:1) to give the title product (134 mg). MS (ESI, m/e) [M+H]⁺ 347.1.

Step 3: 5-chloro-4-fluoro-10,11-dimethyl-2-(methylthio)-8,9,10,11-tetrahydro-7-oxa-1,3,6,11-tetraazacycloocta[de]naphthalene

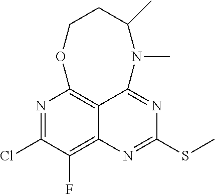 [SMILES: CSc1nc2c3c(nc(Cl)c(F)c3n1)OCCC(C)N2C]

To a solution of 7-chloro-8-fluoro-5-(3-(methylamino)butoxy)-2-(methylthio)pyrido[4,3-d]pyrimidin-4-ol (127 mg, 0.366 mmol) in 20 mL DMF was added N,N-Diisopropylethylamine (238 mg, 1.83 mmol) and 2-(7-Azabenzotriazol-1-yl)-N,N,N′,N′-tetramethyluronium hexafluorophosphate (347 mg, 0.915 mmol) at room temperature and it was stirred at room temperature for 1 h. Then, another batch of N,N-Diisopropylethylamine (708 mg, 5.49 mmol) and 2-(7-Azabenzotriazol-1-yl)-N,N,N′,N′-tetramethyluronium hexafluorophosphate (3.47 g, 9.15 mmol) was added and the mixture was stirred at room temperature for 2 hours. The mixture was diluted with water, and extracted with EtOAc. Combined organic layer was dried over sodium sulfate and evaporated. The residue was purified by chromatography column on silica (PE: EtOAc=1:5) to give the title product (92 mg). MS (ESI, m/e) [M+H]⁺ 329.1.

Step 4: 5-chloro-4-fluoro-10,11-dimethyl-2-(methylsulfinyl)-8,9,10,11-tetrahydro-7-oxa-1,3,6,11-tetraazacycloocta[de]naphthalene

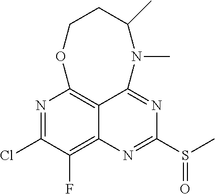 [SMILES: CC1CCOc2nc(Cl)c(F)c3nc(S(C)=O)nc(c23)N1C]

To a solution of 5-chloro-4-fluoro-10,11-dimethyl-2-(methylthio)-8,9,10,11-tetrahydro-7-oxa-1,3,6,11-tetraazacycloocta[de]naphthalene (92 mg, 0.28 mmol) in DCM (10 mL) was added m-CPBA (53 mg, 0.31 mmol) at r.t., and the mixture was stirred at r.t. for 1 h. Upon completion, the mixture was diluted with water (10 mL). Aqueous layer was extracted with DCM (20 mL*3), and the combined organic layer was concentrated to give a residue as the title compound (92 mg). MS (ESI, m/e) [M+H]⁺ 345.1.

Step 5: 5-chloro-4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalene

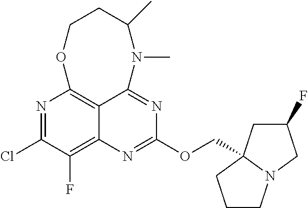 [SMILES: CC1CCOc2nc(Cl)c(F)c3nc(OC[C@@]45CCCN4C[C@H](F)C5)nc(c23)N1C]

To a solution of ((2R,7aS)-2-fluorotetrahydro-1H-pyrrolizin-7a(5H)-yl) methanol (127 mg, 0.8 mmol) in 10 mL THF was added LiHMDS (1 N in THF, 0.5 mL, 0.5 mmol) at room temperature and it was stirred at room temperature for 1 hour. Then 5-chloro-4-fluoro-10,11-dimethyl-2-(methylsulfinyl)-8,9,10,11-tetrahydro-7-oxa-1,3,6,11-tetraazacycloocta[de]naphthalene (92 mg, 0.267 mmol) in THF (10 mL) was added to the reaction mixture and stirred at 0° C. to room temperature for 1 hour. After completion, the reaction mixture was evaporated. The residue was purified by chromatography column on silica (DCM: MeOH=10:1) to give the title product (27 mg). MS (ESI, m/e) [M+H]⁺ 440.1.

Step 6: 3-chloro-5-(4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalen-5-yl)-4-(trifluoromethyl)aniline

To a solution of 5-chloro-4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalene (27 mg, 0.06 mmol) in dioxane/H2O (10/2 mL) was added 3-chloro-5-(4,4,5,5-tetramethyl-1,3,2-dioxaborolan-2-yl)-4-(trifluoromethyl) aniline (77 mg, 0.24 mmol), NaHCO₃ (15 mg, 0.18 mmol) and Pd(dtbpf)Cl₂ (4 mg, 0.006 mmol) at room temperature, and the mixture was stirred at 95° C. for 3 hours. The resulting cooled mixture was concentrated and purified by column chromatography (DCM/MeOH=10/1) to give a residue which was further purified by Prep-HPLC to give the title product (4.2 mg). ¹H NMR (500 MHz, CD₃OD) δ6.88-6.87 (m, 1H), 6.62-6.34 (m, 1H), 5.44-5.27 (m, 1H), 4.49-4.30 (m, 4H), 4.10-3.98 (m, 1H), 3.52-3.33 (m, 3H), 3.29 (s, 3H), 3.15-3.07 (m, 1H), 2.46-1.90 (m, 8H), 1.49-1.39 (m, 3H). MS (ESI, m/e) [M+H]⁺ 599.2.

Example 7: 2-(5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethan-1-ol

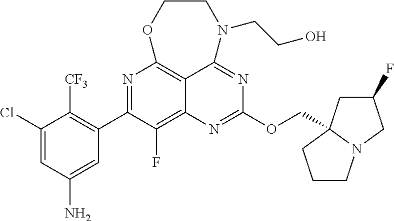 [SMILES: Nc1cc(Cl)c(C(F)(F)F)c(-c2nc3c4c(nc(OC[C@@]56CCCN5C[C@H](F)C6)nc4c2F)N(CCO)CCO3)c1]

Step 1: 4,5,7-trichloro-8-fluoro-2-(methylthio)pyrido[4,3-d]pyrimidine

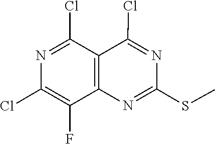 [SMILES: CSc1nc(Cl)c2c(Cl)nc(Cl)c(F)c2n1]

To a solution of 5,7-dichloro-8-fluoro-2-(methylthio)pyrido[4,3-d]pyrimidin-4-ol (1.00 g, 3.57 mmol) in 15 mL acetonitrile was added 0.5 mL N,N-diisopropylethylamine and phosphoryl trichloride (760 mg, 5.00 mmol). The reaction was stirred at 80 ºC for 16 hrs, and it was cooled to room temperature. The mixture was evaporated to give the title product (1,10 g, crude) which was not purified and used in the next step.

Step 2: 2,2′-((5,7-dichloro-8-fluoro-2-(methylthio)pyrido[4,3-d]pyrimidin-4-yl)azanediyl)bis(ethan-1-ol)

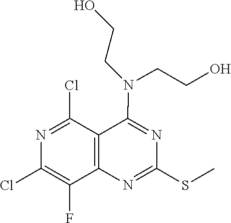 [SMILES: CSc1nc(N(CCO)CCO)c2c(Cl)nc(Cl)c(F)c2n1]

To a solution of 4,5,7-trichloro-8-fluoro-2-(methylthio)pyrido[4,3-d]pyrimidine (crude 500 mg, 1.69 mmol) in 15 mL dichloromethane was added 2,2′-azanediylbis(ethan-1-ol) (210 mg, 2.00 mmol) and 0.5 mL N,N-diisopropylethylamine at -40° C. The reaction was stirred at -40° C. for 4 hrs, and the mixture was diluted with dichloromethane and water and the organic layer was combined, dried over sodium sulfate and evaporated. The crude product (250 mg) was not purified and continued to the next step. MS (ESI, m/e) [M+H]⁺ 367.4.

Step 3: 2-(5-chloro-4-fluoro-2-(methylthio)-8,9-dihydro-10H-7-oxa-1,3,6,10-tetraazacyclohepta[de]naphthalen-10-yl)ethan-1-ol

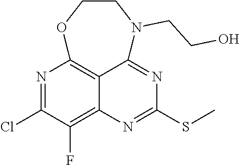 [SMILES: CSc1nc2c3c(nc(Cl)c(F)c3n1)OCCN2CCO]

To a solution of 2,2′-((5,7-dichloro-8-fluoro-2-(methylthio)pyrido[4,3-d]pyrimidin-4-yl)azanediyl)bis(ethan-1-ol) (250 mg, 0.68 mmol) in 10 mL tetrahydrofuran was added sodium hydride (60 mg, 1.50 mmol, 60% w/w) at 0° C. The reaction was stirred at room temperature for 16 hrs, and the mixture was diluted with dichloromethane and water and the organic layer was combined, dried over sodium sulfate and evaporated. The residue was purified by silica gel column to give the title compound (130 mg). MS (ESI, m/e) [M+H]⁺ 331.2.

Step 4: 2-(5-chloro-4-fluoro-2-(methylsulfinyl)-8,9-dihydro-10H-7-oxa-1,3,6,10-tetraazacyclohepta[de]naphthalen-10-yl)ethan-1-ol

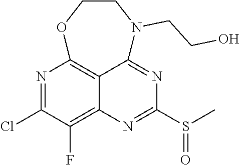 [SMILES: CS(=O)c1nc2c3c(nc(Cl)c(F)c3n1)OCCN2CCO]

To a solution of 2-(5-chloro-4-fluoro-2-(methylthio)-8,9-dihydro-10H-7-oxa-1,3,6,10-tetraazacyclohepta[de]naphthalen-10-yl)ethan-1-ol (130 mg, 0.39 mmol) in 5 mL dichloromethane was added 3-chloroperbenzoic acid (87 mg, 0.50 mmol) at 0° C. The reaction was stirred at 0° C. for 2 hrs, and the mixture was diluted with dichloromethane and water and the organic layer was combined, dried over sodium sulfate and evaporated. The crude product (120 mg) was not purified and continued to the next step. MS (ESI, m/e) [M+H]⁺ 347.0.

Step 5: 2-(5-chloro-4-fluoro-2-(((2R,7aS)-2-fluorotetrahydro-1H-pyrrolizin-7a(5H)-yl)

methoxy)-8,9-dihydro-10H-7-oxa-1,3,6,10-tetraazacyclohepta[de]naphthalen-10-yl)ethan-1-ol

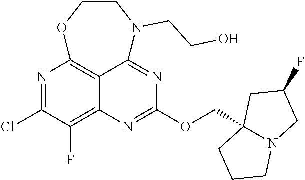 [SMILES: OCCN1CCOc2nc(Cl)c(F)c3nc(OC[C@@]45CCCN4C[C@H](F)C5)nc1c23]

To a solution of 2-(5-chloro-4-fluoro-2-(methylsulfinyl)-8,9-dihydro-10H-7-oxa

-1,3,6,10-tetraazacyclohepta[de]naphthalen-10-yl)ethan-1-ol (crude 120 mg, 0.35 mmol) in 5 mL tetrahydrofuran was added ((2R,7aS)-2-fluorotetrahydro-1H-pyrrolizin-7a(5H)-yl) methanol (160 mg, 1.00 mmol). Then lithium bis(trimethylsilyl)amide (1 mmol/L in tetrahydrofuran, 1.0 mL) was added dropwise at 0° C. The mixture was stirred at 0° C. for 1 h, and the mixture was diluted with dichloromethane and water and the organic layer was combined, dried over sodium sulfate and evaporated. The residue was purified by silica gel column to give the title compound (20 mg). MS (ESI, m/e) [M+H]⁺ 442.0.

Step 6: 2-(5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethan-1-ol

To a solution of 2-(5-chloro-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta[de]naphthalen-10-yl)ethan-1-ol (20 mg, 0.05 mmol) in 2.5 mL 1,4-dioxane and 0.5 mL water was added 3-chloro-5-(4,4,5,5-tetramethyl-1,3-dioxolan-2-yl)-4-(trifluoromethyl)aniline (33 mg, 0.10 mmol), 1,1′-bis (di-t-butylphosphino)ferrocene palladium dichloride (6 mg, 0.01 mmol) and sodium bicarbonate (13 mg, 0.15 mmol). The reaction was stirred at 90° C. for 6 hrs, and the reaction was cooled to room temperature. Then it was diluted with dichloromethane and water and the organic layer was combined, dried over sodium sulfate and evaporated. The residue was purified by Prep-HPLC to give the title compound (0.6 mg). ¹H NMR (500 MHz, DMSO-d₆) δ 6.86-6.85 (m, 1H), 6.45-6.44 (m, 1H), 6.30 (s, 2H), 5.37-5.18 (m, 1H), 4.56-4.50 (m, 2H), 4.13-3.82 (m, 5H), 3.73-3.69 (m, 2H), 3.11-2.99 (m, 3H), 2.87-2.78 (m, 1H), 2.14-1.95 (m, 3H), 1.87-1.72 (m, 3H). MS (ESI, m/e) [M+H]⁺ 601.4.

Example 8: 3-chloro-5-(10-cyclopropyl-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-(trifluoromethyl)aniline

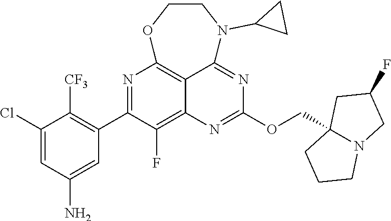 [SMILES: Nc1cc(Cl)c(C(F)(F)F)c(-c2nc3c4c(nc(OC[C@@]56CCCN5C[C@H](F)C6)nc4c2F)N(C2CC2)CCO3)c1]

Step 1: 2-(cyclopropylamino)ethan-1-ol

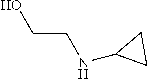

To a solution of 2-bromoethan-1-ol (1.24 g, 10.0 mmol) in 30 mL ethanol was added cyclopropanamine (1.72 g, 30.0 mmol). The reaction was stirred at 60° C. for 16 hrs, and the mixture was cooled to room temperature, which was evaporated to give crude product (1.20 g) which was not purified and continued to the next step.

Step 2: 2-(cyclopropyl(5,7-dichloro-8-fluoro-2-(methylthio)pyrido[4,3-d]pyrimidin-4-yl)amino)ethan-1-ol

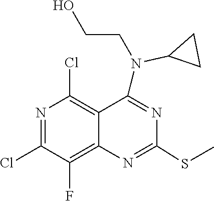 [SMILES: CSc1nc(N(CCO)C2CC2)c2c(Cl)nc(Cl)c(F)c2n1]

To a solution of 4,5,7-trichloro-8-fluoro-2-(methylthio)pyrido[4,3-d]pyrimidine (crude 500 mg, 1.69 mmol) in 15 mL dichloromethane was added 2-(cyclopropylamino)ethan-1-ol (crude 300 mg, 2.97 mmol) and 0.5 mL N,N-diisopropylethylamine at 0° C. The reaction was stirred at room temperature for 4 hrs, and the mixture was diluted with dichloromethane and water. The organic layers were combined, dried over sodium sulfate and evaporated. The crude product was purified by silica gel column to give the title product (80 mg). MS (ESI, m/e) [M+H]⁺ 363.4.

Step 3: 5-chloro-10-cyclopropyl-4-fluoro-2-(methylthio)-9,10-dihydro-8H-7-oxa-1,3,6,10-tetraazacyclohepta[de]naphthalene

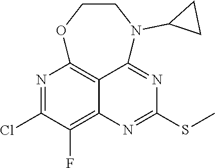 [SMILES: CSc1nc2c3c(nc(Cl)c(F)c3n1)OCCN2C1CC1]

To a solution of 2-(cyclopropyl(5,7-dichloro-8-fluoro-2-(methylthio)pyrido[4,3-d]pyrimidin-4-yl)amino)ethan-1-ol (80 mg, 0.22 mmol) in 10 mL tetrahydrofuran was added sodium hydride (24 mg, 0.60 mmol, 60% w/w) at 0° C. The reaction was stirred at room temperature for 16 hrs, and the mixture was diluted with dichloromethane and water. The organic layer was combined, dried over sodium sulfate and evaporated. The residue was purified by silica gel column to give the title compound (50 mg). MS (ESI, m/e) [M+H]⁺ 327.3.

Step 4: 5-chloro-10-cyclopropyl-4-fluoro-2-(methylsulfinyl)-9,10-dihydro-8H-7-oxa-1,3,6,10-tetraazacyclohepta[de]naphthalene

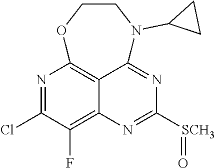

To a solution of 5-chloro-10-cyclopropyl-4-fluoro-2-(methylthio)-9,10-dihydro-8H-7-oxa-1,3,6,10-tetraazacyclohepta[de]naphthalene (50 mg, 0.15 mmol) in 5 mL dichloromethane was added 3-chloroperbenzoic acid (31 mg, 0.20 mmol) at 0° C. The reaction was stirred at 0° C. for 2 hrs, and the mixture was diluted with dichloromethane and water. The organic layer was combined, dried over sodium sulfate and evaporated. The crude product (50 mg) was not purified and continued to the next step. MS (ESI, m/e) [M+H]⁺ 349.2.

Step 5: 5-chloro-10-cyclopropyl-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene

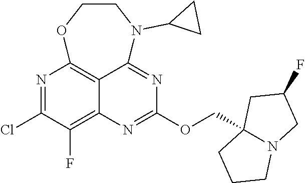 [SMILES: Fc1c(Cl)nc2c3c(nc(OC[C@@]45CCCN4C[C@H](F)C5)nc13)N(C1CC1)CCO2]

To a solution of 5-chloro-10-cyclopropyl-4-fluoro-2-(methylsulfinyl)-9,10-dihydro-8H-7-oxa-1,3,6,10-tetraazacyclohepta[de]naphthalene (crude 50 mg, 0.14 mmol) in 5 mL tetrahydrofuran was added ((2R,7aS)-2-fluorotetrahydro-1H-pyrrolizin-7a(5H)-yl)methanol (80 mg, 0.50 mmol). Then lithium bis(trimethylsilyl)amide (1 mmol L in tetrahydrofuran, 0.5 mL) was added dropwise at 0° C. The mixture was stirred at 0° C. for 1 h, and the mixture was diluted with dichloromethane and water. The organic layer was combined, dried over sodium sulfate and evaporated. The residue was purified by silica gel column to give the title compound (22 mg). MS (ESI, m e) [M+H]⁺ 438.0.

Step 6: 3-chloro-5-(10-cyclopropyl-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)-4-(trifluoromethyl)aniline

To a solution of 5-chloro-10-cyclopropyl-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene (22 mg, 0.05 mmol) in 2.5 mL 1,4-dioxane and 0.5 mL water was added 3-chloro-5-(4,4,5,5-tetramethyl-1,3-dioxolan-2-yl)-4-(trifluoromethyl)aniline (33 mg, 0.10 mmol), 1,1′-bis (di-t-butylphosphino)ferrocene palladium dichloride (6 mg, 0.01 mmol) and sodium bicarbonate (13 mg, 0.15 mmol). The reaction was stirred at 90 ºC for 6 hrs, and the reaction was cooled to room temperature. Then it was diluted with dichloromethane and water and the organic layer was combined, dried over sodium sulfate and evaporated. The residue was purified by Prep-HPLC to give the title compound (0.7 mg). ¹H NMR (500 MHz, DMSO-d₆) δ 6.86-6.84 (m, 1H), 6.47-6.44 (m, 1H), 6.29 (s. 2H), 5.37-5.20 (m, 1H), 4.55-4.47 (m, 2H), 4.20-4.07 (m, 2H), 3.96-3.91 (m, 2H), 3.15-3.00 (m, 4H), 2.88-2.79 (m, 1H), 2.17-1.97 (m, 3H), 1.88-1.75 (m, 3H), 0.95-0.90 (m, 2H), 0.80-0.74 (m, 2H). MS (ESI, m/e) [M+H]⁺ 597.5.

Example 9: 3-chloro-5-(4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalen-5-yl)-4-(trifluoromethyl)aniline

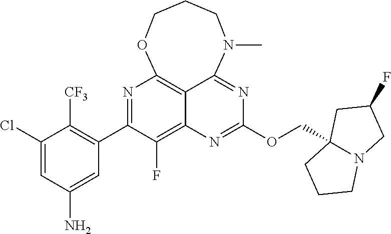 [SMILES: CN1CCCOc2nc(-c3cc(N)cc(Cl)c3C(F)(F)F)c(F)c3nc(OC[C@@]45CCCN4C[C@H](F)C5)nc1c23]

Step 1: 3-((5,7-dichloro-8-fluoro-2-(methylthio)pyrido[4,3-d]pyrimidin-4-yl)(methyl)amino)propan-1-ol

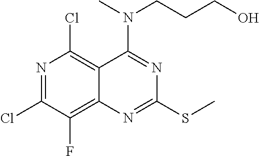 [SMILES: CSc1nc(N(C)CCCO)c2c(Cl)nc(Cl)c(F)c2n1]

To a stirred the solution of 4,5,7-trichloro-8-fluoro-2-(methylthio)pyrido[4,3-d]pyrimidine (1.0 mmol) in DCM (10 mL) was added DIEA (387 mg, 3.0 mmol) and 3-(methylamino)propan-1-ol (89 mg, 1.0 mmol) at 0° C., and the resulting mixture was stirred for 30 mins at room temperature. The reaction mixture was concentrated and purified by flash chromatography (PE/EtOAc=4:1 to 1:2) to afford the desired product (213 mg). MS (ESI, m/e) [M+H]⁺ 351.1.

Step 2: 5-chloro-4-fluoro-11-methyl-2-(methylthio)-8,9,10,11-tetrahydro-7-oxa-1,3,6,11-tetraazacycloocta[de]naphthalene

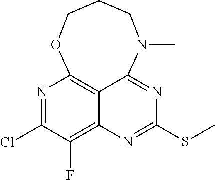 [SMILES: CSc1nc2c3c(nc(Cl)c(F)c3n1)OCCCN2C]

To a stirred the solution of 3-((5,7-dichloro-8-fluoro-2-(methylthio)pyrido[4,3-d]pyrimidin-4-yl)(methyl)amino)propan-1-ol (213 mg, 0.61 mmol) in THF (6 mL) was added NaH (48 mg, 1.2 mmol, 60%) at 0° C., and the resulting mixture was stirred at room temperature for 2 hrs. The reaction was quenched with H₂O, then extracted with EtOAc. The organic layer was washed with brine, dried over anhydrous Na₂SO₄, concentrated in vacuo. The residue was purified by flash chromatography (PE/EtOAc=10:1 to 1:1) to afford the product (186 mg). MS (ESI, m/e) [M+H]⁺ 315.3.

Step 3: 5-chloro-4-fluoro-11-methyl-2-(methylsulfinyl)-8,9,10,11-tetrahydro-7-oxa-1,3,6,11-tetraazacycloocta[de]naphthalene

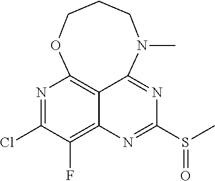 [SMILES: CN1CCCOc2nc(Cl)c(F)c3nc(S(C)=O)nc1c23]

To a stirred the solution of 5-chloro-4-fluoro-11-methyl-2-(methylthio)-8,9,10,11-tetrahydro-7-oxa-1,3,6,11-tetraazacycloocta[de]naphthalene (120 mg, 0.38 mmol) in DCM (6 mL) was added m-CPBA (93 mg, 0.46 mmol, 85%) at 0° C., and the resulting mixture was stirred at 0° C. for 10 min. The reaction was quenched with aq. Na₂S₂O₃, then extracted with DCM. The organic layer was washed with Sat, aq NaHCO₃ and brine, dried over anhydrous Na₂SO₄, concentrated in vacuo. The residue was directly used in the next step without further purification. MS (ESI, m e) [M+H]⁺ 331,1.

Step 4: 5-chloro-4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene

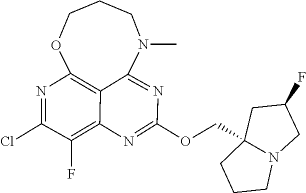 [SMILES: CN1CCCOc2nc(Cl)c(F)c3nc(OC[C@@]45CCCN4C[C@H](F)C5)nc1c23]

To a stirred the solution of 5-chloro-4-fluoro-11-methyl-2-(methylsulfinyl)-8,9,10,11-tetrahydro-7-oxa-1,3,6,11-tetraazacycloocta[de]naphthalene (0.38 mmol) and ((2R,7aS)-2-fluorotetrahydro-1H-pyrrolizin-7a(5H)-yl)methanol (120 mg, 0.76 mmol) in THF (6 mL) was added LiHMDS (0.76 mL, 0.76 mmol, 1M in THF) dropwise at 0° C., and the resulting mixture was stirred at 0° C. for 20 min. The reaction was quenched with Sat, aq NH₄Cl, then extracted with EtOAc. The organic layer was washed with brine, dried over anhydrous Na₂SO₄, concentrated in vacuo. The residue was purified by flash chromatography (DCM/MeOH=100:1 to 30:1) to afford the product (125 mg). MS (ESI, m/e) [M+H]⁺ 426.2.

Step 5: 3-chloro-5-(4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalen-5-yl)-4-(trifluoromethyl)aniline

To a mixture of 5-chloro-4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene (25 mg, 0.06 mmol), 3-chloro-5-(4,4,5,5-tetramethyl-1,3,2-dioxaborolan-2-yl)-4-(trifluoromethyl)aniline (43 mg, 0.12 mmol, 89%), Pd(dtbpf)Cl₂ (7.8 mg, 0.012 mmol) and NaHCO₃ (15 mg, 0.18 mmol) was added dioxane (2.0 mL) and H₂O (0.4 mL). The reaction mixture was stirred at 90° C. for 2 hrs. The reaction mixture was concentrated and purified by flash chromatography (DCM/MeOH=100:1 to 5:1), then prep-HPLC to give the title product (6.2 mg). ¹H NMR (500 MHZ, CD₃OD) δ6.87 (s, 1H), 6.50 (s, 1H), 5.42-5.24 (m, 1H), 4.52-4.21 (m, 4H), 3.73-3.62 (m, 2H), 3.40 (s, 3H), 3.11-3.02 (m, 1H), 2.43-1.85 (m, 8H). MS (ESI, m/e) [M+H]⁺ 585.4.

Example 10: 5-ethynyl-6-fluoro-4-(4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalen-5-yl)naphthalen-2-ol

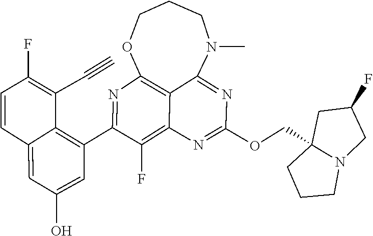 [SMILES: C#Cc1c(F)ccc2cc(O)cc(-c3nc4c5c(nc(OC[C@@]67CCCN6C[C@H](F)C7)nc5c3F)N(C)CCCO4)c12]

Step 1: 4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene

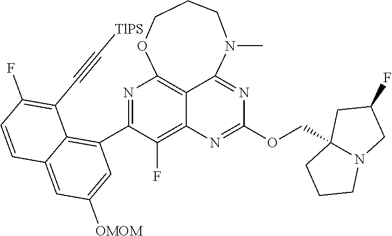

To a mixture of 5-chloro-4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene (50 mg, 0.12 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (123 mg, 0.24 mmol), Pd(dtbpf)Cl₂ (15.6 mg, 0.024 mmol) and NaHCO₃(30 mg, 0.36 mmol) was added dioxane (5.0 mL) and H₂O (1.0 mL). The reaction mixture was stirred at 90° C. for 2 hrs. The reaction mixture was concentrated and purified by flash chromatography (DCM/MeOH=100:1 to 10:1) to give the title product (75 mg). MS (ESI, m/e) [M+H]⁺ 776.5.

Step 2: 5-(8-ethynyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene

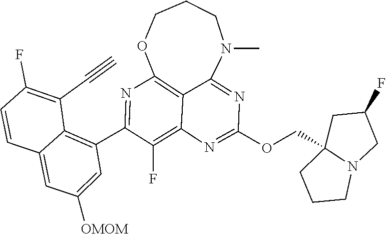

To a stirred the solution of 4-fluoro-5-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene (75 mg, 0.10 mmol) in DMF (3 mL) was added CsF (152 mg, 1.0 mmol), and the resulting mixture was stirred for 2 hrs at room temperature. The reaction mixture was diluted with EtOAc and H₂O, then extracted with EtOAc. The organic layer was washed with brine, dried over anhydrous Na₂SO₄, and concentrated in vacuo. The crude was directly used in the next step without further purification. MS (ESI, m/e) [M+H]⁺ 620.3.

Step 3: 5-ethynyl-6-fluoro-4-(4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalen-5-yl)naphthalen-2-ol

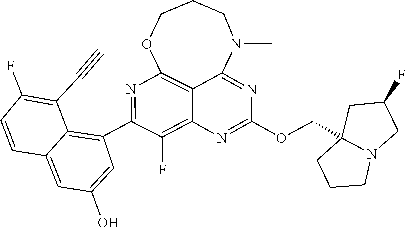

To a stirred the solution of 5-(8-ethynyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-11-methyl-8,9,10,11-tetrahydro-7-oxa-1,3,6,11-tetraazacycloocta[de]naphthalene (0.10 mmol) in DCM (5.0 mL) was added 4N HCl (in dioxane) (1.0 mL) at 0° C., and the resulting mixture was stirred at room temperature for 1 h. The reaction mixture was concentrated and basified with DIEA, and the residue was purified by flash chromatography (DCM/MeOH=100:1 to 10:1), then Prep-HPLC to afford the product (19.7 mg). ¹H NMR (500 MHZ, CD₃OD) δ7.87-7.78 (m, 1H), 7.37-7.21 (m, 3H), 5.55-5.32 (m, 1H), 4.58-4.35 (m, 4H), 3.83-3.50 (m, 6H), 3.44 (s, 3H), 2.60-1.97 (m, 8H). MS (ESI, m/e) [M+H]⁺ 576.8.

Example 11: 3-chloro-5-(4-fluoro-2-(((2R,7aS)-2-fluorotetrahydro-1H-pyrrolizin-7a(5H)-yl)methoxy)-11-methyl-10,11-dihydro-8H-7-oxa-1,3,6,11-tetraazaspiro[cycloocta[de]naphthalene-9,3′-oxetan]-1(11a), 2,3a, 3 al(6a), 5-pentaen-5-yl)-4-(trifluoromethyl)aniline

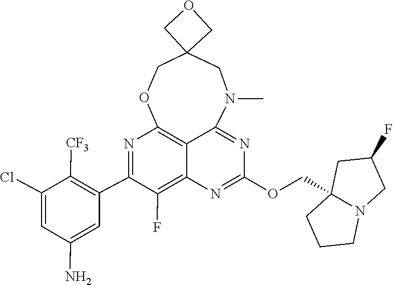 [SMILES: CN1CC2(COC2)COc2nc(-c3cc(N)cc(Cl)c3C(F)(F)F)c(F)c3nc(OC[C@@]45CCCN4C[C@H](F)C5)nc1c23]

Step 1: (3-(((5,7-dichloro-8-fluoro-2-(methylthio)pyrido[4,3-d]pyrimidin-4-yl)(methyl)amino)methyl)oxetan-3-yl)methanol

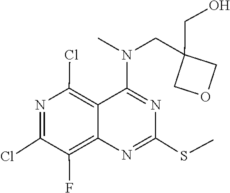 [SMILES: CSc1nc(N(C)CC2(CO)COC2)c2c(Cl)nc(Cl)c(F)c2n1]

To a solution of 4,5,7-trichloro-8-fluoro-2-(methylthio) pyrido[4,3-d]pyrimidine (0.85 g, 2.9 mmol) in DCM (30 mL) was added DIPEA (1,1 g, 0.85 mmol) and (3-((methylamino)methyl)oxetan-3-yl)methanol (374 mg, 2.9 mmol) at 0° C. The resulting mixture was stirred at 0° C. to room temperature for 1 hour. After completion, the reaction mixture was concentrated to give the residue. The residue was purified by flash column silica (PE: EtOAc=2:1) to give the title product (600 mg). MS (ESI, m/e) [M+H]⁺ 393.3.

Step 2: 5-chloro-4-fluoro-11-methyl-2-(methylthio)-10,11-dihydro-8H-7-oxa-1,3,6,11-tetraazaspiro[cycloocta[de]naphthalene-9,3′-oxetan]-1(11a), 2,3a, 3 al(6a), 5-pentaene

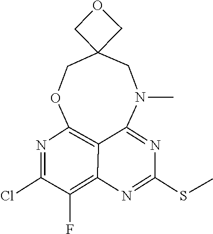 [SMILES: CSc1nc2c3c(nc(Cl)c(F)c3n1)OCC1(COC1)CN2C]

To a solution of (3-(((5,7-dichloro-8-fluoro-2-(methylthio)pyrido[4,3-d]pyrimidin-4-yl)(methyl)amino)methyl)oxetan-3-yl)methanol (600 mg, 1.5 mmol) in THF (25 mL) was added LiHMDS (1 N in THF) (1.5 mL, 1.5 mmol) at room temperature, and the mixture was stirred at r.t. for 2 hrs. Upon completion, the mixture was evaporated. The residue was purified by chromatography column on silica (PE: EtOAc=1:1) to give the title product (450 mg). MS (ESI, m/e) [M+H]⁺ 357.1.

Step 3: 5-chloro-4-fluoro-2-(((2R,7aS)-2-fluorotetrahydro-1H-pyrrolizin-7a(5H)-yl)methoxy)-11-methyl-10,11-dihydro-8H-7-oxa-1,3,6,11-tetraazaspiro[cycloocta[de]naphthalene-9,3′-oxetan]-1(11a), 2,3a, 3a¹(6a), 5-pentaene

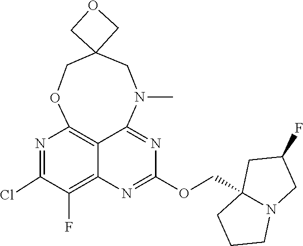 [SMILES: CN1CC2(COC2)COc2nc(Cl)c(F)c3nc(OC[C@@]45CCCN4C[C@H](F)C5)nc1c23]

To a solution of 5-chloro-4-fluoro-11-methyl-2-(methylthio)-10,11-dihydro-8H-7-oxa-1,3,6,11-tetraazaspiro[cycloocta[de]naphthalene-9,3′-oxetan]-1(11a), 2,3a, 3a¹(6a), 5-pentaene (450 mg, 1.26 mmol) in DCM (20 mL) was added m-CPBA (231 mg, 1.51 mmol) at rt., and the mixture was stirred at rt. for 1 h as solution 1. Meanwhile, to a solution of ((2R,7aS)-2-fluorotetrahydro-1H-pyrrolizin-7a(5H)-yl) methanol (602 mg, 3.79 mmol) in 20 mL THF was added LiHMDS (1 N in THF, 2.5 mL, 2.5 mmol) at room temperature and it was stirred at room temperature for 1 hour as solution 2. Then the solution 2 was added to solution 1 at room temperature and the mixture was stirred at room temperature for 2 hours. After completion, the reaction mixture was evaporated. The residue was purified by chromatography column on silica (DCM: MeOH=10:1) to give the title product (180 mg). MS (ESI, m/e) [M+H]⁺ 468.2.

Step 4: 3-chloro-5-(4-fluoro-2-(((2R,7aS)-2-fluorotetrahydro-1H-pyrrolizin-7a(5H)-yl)methoxy)-11-methyl-10,11-dihydro-8H-7-oxa-1,3,6,11-tetraazaspiro[cycloocta[de]naphthalene-9,3′-oxetan]-1(11a), 2,3a, 3a¹(6a), 5-pentaen-5-yl)-4-(trifluoromethyl)aniline

To a solution of 5-chloro-4-fluoro-2-(((2R,7aS)-2-fluorotetrahydro-1H-pyrrolizin-7a(5H)-yl)methoxy)-11-methyl-10,11-dihydro-8H-7-oxa-1,3,6,11-tetraazaspiro[cycloocta[de]naphthalene-9,3′-oxetan]-1(11a), 2,3a, 3a¹(6a), 5-pentaene (60 mg, 0.128 mmol) in dioxane/H₂O (10/2 mL) was added 3-chloro-5-(4,4,5,5-tetramethyl-1,3,2-dioxaborolan-2-yl)-4-(trifluoromethyl)aniline (82 mg, 0.256 mmol), NaHCO₃(2.5 mg, 0.256 mmol) and Pd(dtbpf)Cl₂ (16.6 mg, 0.0256 mmol) at room temperature, and the mixture was stirred at 100° C. for 3 hours. The resulting cooled mixture was concentrated and purified by column chromatography (DCM/MeOH=10/1) to give a residue which was further purified by Prep-HPLC to give the title product (4.43 mg). ¹H NMR (500 MHZ, CD₃OD) δ6.96-6.81 (m, 1H), 6.55-6.40 (m, 1H), 5.48-5.26 (m, 1H), 4.74-4.56 (m, 5H), 4.40-4.26 (m, 2H), 4.01-3.92 (m, 2H), 3.45-3.32 (m, 3H), 3.13-3.05 (m, 1H), 2.41-1.88 (m, 8H). MS (ESI, m/e) [M+H]⁺ 327.2.

Example 12: 3-chloro-5-(4-fluoro-2-(((2R,7aS)-2-fluorotetrahydro-1H-pyrrolizin-7a(5H)-yl)methoxy)-8,10-dimethyl-9,10-dihydro-8H-7-oxa-1,3,6,10-tetraazacyclohepta[de]naphthalen-5-yl)-4-(trifluoromethyl)aniline

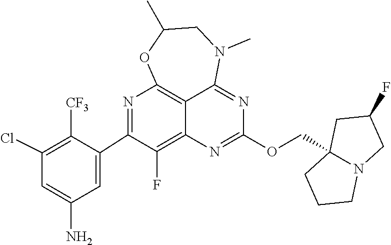 [SMILES: CC1CN(C)c2nc(OC[C@@]34CCCN3C[C@H](F)C4)nc3c(F)c(-c4cc(N)cc(Cl)c4C(F)(F)F)nc(c23)O1]

Step 1: 1-(methylamino) propan-2-ol

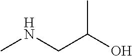

To a solution of tert-butyl (2-hydroxypropyl) (methyl)carbamate (440 mg, 2.33 mmol) in DCM (10 mL) was added TFA (5 mL) at room temperature, and the mixture was stirred at r.t. for 2 hrs. Upon completion, the mixture was evaporated to get the title product (300 mg, crude). MS (ESI, m/e) [M+H]⁺ 90.1.

Step 2: 1-((5,7-dichloro-8-fluoro-2-(methylthio) pyrido[4,3-d]pyrimidin-4-yl) (methyl)amino) propan-2-ol

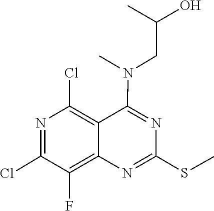 [SMILES: CSc1nc(N(C)CC(C)O)c2c(Cl)nc(Cl)c(F)c2n1]

To a solution of 4,5,7-trichloro-8-fluoro-2-(methylthio) pyrido[4,3-d]pyrimidine (339 mg. 1,17 mmol) in DCM (30 mL) was added DIPEA (350 mg, 2.91 mmol) and 1-(methylamino) propan-2-ol (150 mg, 1,17 mmol) at 0° C. The resulting mixture was stirred at 0° C. to room temperature for 1 hour. After completion, the reaction mixture was concentrated to give the residue. The residue was purified by flash column silica (PE: EtOAc=1:1) to give the title product (160 mg). MS (ESI, m/e) [M+H]⁺ 351.1.

Step 3: 5-chloro-4-fluoro-8,10-dimethyl-2-(methylthio)-9,10-dihydro-8H-7-oxa-1,3,6,10-tetraazacyclohepta[de]naphthalene

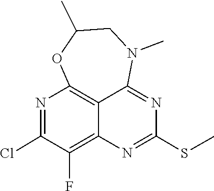 [SMILES: CSc1nc2c3c(nc(Cl)c(F)c3n1)OC(C)CN2C]

To a solution of 1-((5,7-dichloro-8-fluoro-2-(methylthio) pyrido[4,3-d]pyrimidin-4-yl) (methyl)amino) propan-2-ol (160 mg, 0.46 mmol) in THF (25 mL) was added NaH (37 mg, 0.92 mmol) at room temperature, and the mixture was stirred at r.t. for 2 hrs. Upon completion, the mixture was evaporated. The residue was purified by chromatography column on silica (PE: EtOAc=1:1) to give the title product (63 mg). MS (ESI, m/e) [M+H]⁺ 315.1.

Step 4: 5-chloro-4-fluoro-2-(((2R,7aS)-2-fluorotetrahydro-1H-pyrrolizin-7a(5H)-yl) methoxy)-8,10-dimethyl-9,10-dihydro-8H-7-oxa-1,3,6,10-tetraazacyclohepta[de]naphthalene

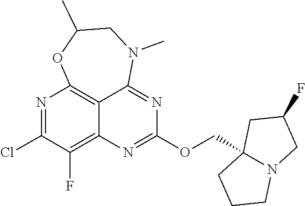 [SMILES: CC1CN(C)c2nc(OC[C@@]34CCCN3C[C@H](F)C4)nc3c(F)c(Cl)nc(c23)O1]

To a solution of 5-chloro-4-fluoro-8,10-dimethyl-2-(methylthio)-9,10-dihydro-8H-7-oxa-1,3,6,10-tetraazacyclohepta[de]naphthalene (63 mg, 0.2 mmol) in DCM (20 mL) was added m-CPBA (34.5 mg, 0.2 mmol) at rt., and the mixture was stirred at rt. for 1 h as solution 1. Meanwhile, to a solution of ((2R,7aS)-2-fluorotetrahydro-1H-pyrrolizin-7a(5H)-yl) methanol (95.4 mg, 0.6 mmol) in 20 mL THF was added LiHMDS (1 N in THF, 0.4 mL, 0.4 mmol) at room temperature and it was stirred at room temperature for 1 hour as the solution 2. Then the solution 2 was added to solution 1 at room temperature and the mixture was stirred at room temperature for 2 hours. After completion, the reaction mixture was evaporated. The residue was purified by chromatography column on silica (DCM: MeOH=10:1) to give the title product (80 mg, crude). MS (ESI, m/e) [M+H]⁺ 426.1.

Step 5: 3-chloro-5-(4-fluoro-2-(((2R,7aS)-2-fluorotetrahydro-1H-pyrrolizin-7a(5H)-yl)methoxy)-8,10-dimethyl-9,10-dihydro-8H-7-oxa-1,3,6,10-tetraazacyclohepta[de]naphthalen-5-yl)-4-(trifluoromethyl)aniline

To a solution of 5-chloro-4-fluoro-2-(((2R,7aS)-2-fluorotetrahydro-1H-pyrrolizin-7a(5H)-yl) methoxy)-8,10-dimethyl-9,10-dihydro-8H-7-oxa-1,3,6,10-tetraazacyclohepta[de]naphthalene (42.5 mg, 0.1 mmol) in dioxane/H₂O(10/2 mL) was added 3-chloro-5-(4,4,5,5-tetramethyl-1,3,2-dioxaborolan-2-yl)-4-(trifluoromethyl)aniline (80 mg, 0.25 mmol), NaHCO₃(25.2 mg, 0.3 mmol) and Pd(dtbpf)Cl₂ (6.5 mg, 0.01 mmol) at room temperature, and the mixture was stirred at 95° C. for 3 hours. The resulting cooled mixture was concentrated and purified by column chromatography (DCM/MeOH=10/1) to give a residue which was further purified by Prep-HPLC to give the title product (5.8 mg). ¹H NMR (500 MHZ, CD₃OD) δ6.93-6.91 (m, 1H), 6.58-6.46 (m, 1H), 5.47-5.28 (m, 1H), 4.78-4.72 (m, 1H), 4.39-4.30 (m, 2H), 4.01-3.89 (m, 2H), 3.48 (s, 3H), 3.13-3.06 (m, 1H), 2.46-1.94 (m, 6H), 1.60-1.52 (m, 3H). MS (ESI, m/e) [M+H]⁺ 585.2.

Example 13: 4-(11-cyclobutyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalen-5-yl)-5-ethynyl-6-fluoronaphthalen-2-ol

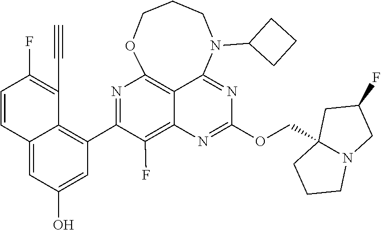 [SMILES: C#Cc1c(F)ccc2cc(O)cc(-c3nc4c5c(nc(OC[C@@]67CCCN6C[C@H](F)C7)nc5c3F)N(C3CCC3)CCCO4)c12]

Step 1: 3-(cyclobutylamino) propan-1-ol

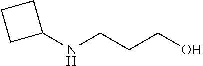

Cyclobutanamine (710 mg, 10 mmol) was mixed with 3-bromopropan-1-ol (278 mg, 2 mmol) at room temperature, and the mixture was stirred at 50° C. overnight. Upon completion, the mixture was concentrated to give a residue as the title compound (500 mg crude). MS (ESI, m/e) [M+H]⁺ 130.1.

Step 2: 3-(cyclobutyl(5,7-dichloro-8-fluoro-2-(methylthio) pyrido[4,3-d]pyrimidin-4-yl) amino) propan-1-ol

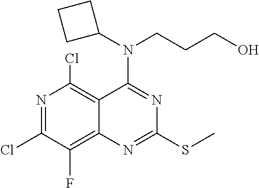 [SMILES: CSc1nc(N(CCCO)C2CCC2)c2c(Cl)nc(Cl)c(F)c2n1]

To a solution of 4,5,7-trichloro-8-fluoro-2-(methylthio) pyrido[4,3-d]pyrimidine (330 mg, 1,11 mmol) in DCM (20 mL) was added DIPEA (358 mg, 2.78 mmol) and 3-(cyclobutylamino) propan-1-ol (258 mg, 1 mmol) at 0° C. The resulting mixture was stirred at 0° C. to room temperature for 1 hour. After completion, the reaction mixture was concentrated to give the residue. The residue was purified by flash column silica (PE: EtOAc=1:1) to give the title product (230 mg). MS (ESI, m/e) [M+H]⁺ 390.1.

Step 3: 5-chloro-11-cyclobutyl-4-fluoro-2-(methylthio)-8,9,10,11-tetrahydro-7-oxa-1,3,6,11-tetraazacycloocta[de]naphthalene

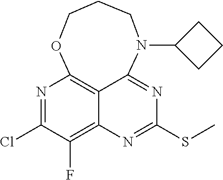 [SMILES: CSc1nc2c3c(nc(Cl)c(F)c3n1)OCCCN2C1CCC1]

To a solution of 3-(cyclobutyl(5,7-dichloro-8-fluoro-2-(methylthio) pyrido[4,3-d]pyrimidin-4-yl) amino) propan-1-ol (350 mg, 0.9 mmol) in THF (25 mL) was added NaH (72 mg, 1.8 mmol) at room temperature, and the mixture was stirred at r.t. for 2 hrs. Upon completion, the mixture was quenched with 2 drops of ice water and the mixture was evaporated. The residue was purified by chromatography column on silica (PE: EtOAc=3:1) to give the title product (280 mg crude). MS (ESI, m/e) [M+H]⁺ 355.1.

Step 4: 5-chloro-11-cyclobutyl-4-fluoro-2-(methylsulfinyl)-8,9,10,11-tetrahydro-7-oxa-1,3,6,11-tetraazacycloocta[de]naphthalene

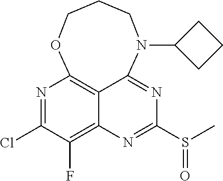 [SMILES: CS(=O)c1nc2c3c(nc(Cl)c(F)c3n1)OCCCN2C1CCC1]

To a solution of 5-chloro-11-cyclobutyl-4-fluoro-2-(methylthio)-8,9,10,11-tetrahydro-7-oxa-1,3,6,11-tetraazacycloocta[de]naphthalene (280 mg, 0.79 mmol) in DCM (20 mL) was added m-CPBA (163.5 mg, 0.95 mmol) at r.t., and the mixture was stirred at r.t. for 1 h. Upon completion, the mixture was diluted with water (10 mL). Aqueous layer was extracted with DCM (20 mL*3), and the combined organic layer was concentrated to give a residue as the title compound (330 mg crude). MS (ESI, m/e) [M+H]⁺ 371,1

Step 5: 5-chloro-11-cyclobutyl-4-fluoro-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene

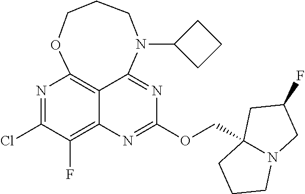 [SMILES: Fc1c(Cl)nc2c3c(nc(OC[C@@]45CCCN4C[C@H](F)C5)nc13)N(C1CCC1)CCCO2]

To a solution of ((2R,7aS)-2-fluorotetrahydro-1H-pyrrolizin-7a(5H)-yl) methanol (167 mg, 1.05 mmol) in 20 mL THF was added LiHMDS (1 N in THF, 0.7 mL, 0.7 mmol) at room temperature and it was stirred at room temperature for 1 hour. Then, a solution of 5-chloro-11-cyclobutyl-4-fluoro-2-(methylsulfinyl)-8,9,10,11-tetrahydro-7-oxa-1,3,6,11-tetraazacycloocta[de]naphthalene (130 mg, 0.35 mmol) in THF (10 mL) was added to the reaction mixture and the mixture was stirred at 0° C. to room temperature for 1 hour. After completion, the reaction mixture was evaporated. The residue was purified by chromatography column on silica (DCM: MeOH=10:1) to give the title product (78 mg crude). MS (ESI, m/e) [M+H]⁺ 466.1.

Step 6: 11-cyclobutyl-4-fluoro-5-(7-fluoro-3-(methoxymethoxy)-8-((triisopropylsilyl)ethynyl) naphthalen-1-yl)-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene

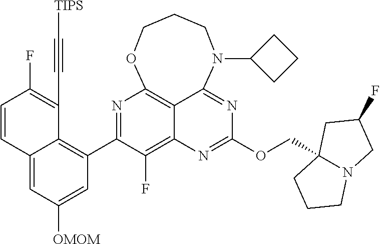

To a solution of 5-chloro-11-cyclobutyl-4-fluoro-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene (48 mg, 0.1 mmol) in dioxane/H₂O (10/2 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (205 mg, 0.4 mmol), NaHCO₃(25 mg, 0.3 mmol) and Pd(dtbpf)Cl₂(6.5 mg, 0.01 mmol) at room temperature, and the mixture was stirred at 95° C. for 3 hours. Upon completion, the mixture was evaporated and purified by chromatography column on silica (DCM: MeOH=20:1) to get the title product (67 mg crude). MS (ESI, m/e) [M+H]⁺ 816.4.

Step 7: 11-cyclobutyl-5-(8-ethynyl-7-fluoro-3-(methoxymethoxy) naphthalen-1-yl)-4-fluoro-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene

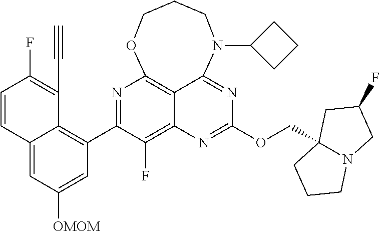

To a solution of 11-cyclobutyl-4-fluoro-5-(7-fluoro-3-(methoxymethoxy)-8-((triisopropylsilyl)ethynyl) naphthalen-1-yl)-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene (67 mg, 0.082 mmol) in DMF (2 mL) was added CsF (62 mg, 0.41 mmol) at room temperature. Upon completion, the mixture was diluted with water (10 mL). Aqueous layer was extracted with DCM (20 mL*3), and the combined organic layer was concentrated to give a residue as the title compound (66 mg crude). MS (ESI, m/e) [M+H]⁺ 660.3.

Step 7: 4-(11-cyclobutyl-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalen-5-yl)-5-ethynyl-6-fluoronaphthalen-2-ol

To a solution of 11-cyclobutyl-5-(8-ethynyl-7-fluoro-3-(methoxymethoxy) naphthalen-1-yl)-4-fluoro-2-(((2R,7aS)-2-fluorotetrahydro-1H-pyrrolizin-7a(5H)-yl) methoxy)-8,9,10,11-tetrahydro-7-oxa-1,3,6,11-tetraazacycloocta[de]naphthalene (33 mg, 0.05 mmol) in EtOAc (5 mL) was added HCl (4 N, 1 mL) at room temperature. The resulting mixture was concentrated at room temperature and pH was adjusted to 7 with Na₂CO₃, and the organic layer was concentrated to give a residue which was further purified by Prep-HPLC to give the title product (5.2 mg). ¹H NMR (500 MHZ, CD₃OD) δ7.89-7.82 (m. 1H), 7.40-7.25 (m, 3H), 5.63-5.42 (m, 1H), 5.22-5.14 (m, 1H), 4.71-4.44 (m, 4H), 4.02-3.40 (m, 7H), 2.73-1.83 (m, 14H). MS (ESI, m/e) [M+H]⁺ 616.3.

Example 14: 3-chloro-5-(4-fluoro-2-(((2R,7aS)-2-fluorotetrahydro-1H-pyrrolizin-7a(5H)-yl)methoxy)-8,11-dimethyl-8,9,10,11-tetrahydro-7-oxa-1,3,6,11-tetraazacycloocta[de]naphthalen-5-yl)-4-(trifluoromethyl)aniline

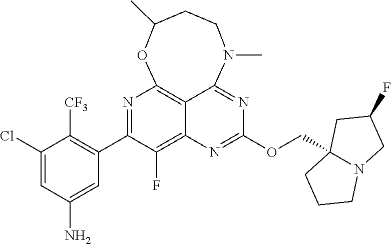 [SMILES: CC1CCN(C)c2nc(OC[C@@]34CCCN3C[C@H](F)C4)nc3c(F)c(-c4cc(N)cc(Cl)c4C(F)(F)F)nc(c23)O1]

Step 1: 4-((5,7-dichloro-8-fluoro-2-(methylthio)pyrido[4,3-d]pyrimidin-4-yl)(methyl)amino)butan-2-ol

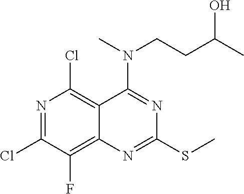 [SMILES: CSc1nc(N(C)CCC(C)O)c2c(Cl)nc(Cl)c(F)c2n1]

To a solution of 4,5,7-trichloro-8-fluoro-2-(methylthio) pyrido[4,3-d]pyrimidine (0.85 g, 2.9 mmol) in DCM (30 mL) was added DIPEA (1,1 g, 0.85 mmol) and 4-(methylamino)butan-2-ol (346 mg, 2.9 mmol) at 0° C. The resulting mixture was stirred at 0° C. to room temperature for 1 hour. After completion, the reaction mixture was concentrated to give the residue. The residue was purified by flash column silica (PE: EtOAc -2:1) to give the title product (450 mg). MS (ESI, m/e) [M−H]˜ 365.2.

Step 2: 5-chloro-4-fluoro-8,11-dimethyl-2-(methylthio)-8,9,10,11-tetrahydro-7-oxa-1,3,6,11-tetraazacycloocta[de]naphthalene

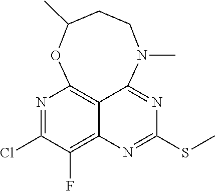 [SMILES: CSc1nc2c3c(nc(Cl)c(F)c3n1)OC(C)CCN2C]

To a solution of 4-((5,7-dichloro-8-fluoro-2-(methylthio)pyrido[4,3-d]pyrimidin-4-yl)(methyl)amino)butan-2-ol (450 mg, 1.23 mmol) in THF (25 mL) was added NaH (60% in oil, 49.5 mg, 1.23 mmol) at room temperature, and the mixture was stirred at r.t. for 2 hrs. Upon completion, the mixture was evaporated. The residue was purified by chromatography column on silica (PE: EtOAc=1:1) to give the title product (100 mg). MS (ESI, m/e) [M+H]⁺ 329.1.

Step 3: 5-chloro-4-fluoro-2-(((2R,7aS)-2-fluorotetrahydro-1H-pyrrolizin-7a(5H)-yl)methoxy)-8,11-dimethyl-8,9,10,11-tetrahydro-7-oxa-1,3,6,11-tetraazacycloocta[de]naphthalene

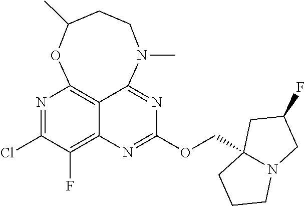 [SMILES: CC1CCN(C)c2nc(OC[C@@]34CCCN3C[C@H](F)C4)nc3c(F)c(Cl)nc(c23)O1]

To a solution of 5-chloro-4-fluoro-8,11-dimethyl-2-(methylthio)-8,9,10,11-tetrahydro-7-oxa-1,3,6,11-tetraazacycloocta[de]naphthalene (100 mg, 0.3 mmol) in DCM (20 mL) was added m-CPBA (56 mg, 0.36 mmol) at rt., and the mixture was stirred at rt. for 1 h as solution 1. Meanwhile, to a solution of ((2R,7aS)-2-fluorotetrahydro-1H-pyrrolizin-7a(5H)-yl) methanol (145 mg, 0.9 mmol) in 20 mL THF was added LiHMDS (1 N in THF, 0.6 mL, 0.6 mmol) at room temperature and it was stirred at room temperature for 1 hour as the solution 2. Then, the solution 2 was added to solution 1 at room temperature and the mixture was stirred at room temperature for 2 hours. After completion, the reaction mixture was evaporated. The residue was purified by chromatography column on silica (DCM: MeOH=10:1) to give the title product (40 mg). MS (ESI, m e) [M+H]: 440.2.

Step 4: 3-chloro-5-(4-fluoro-2-(((2R,7aS)-2-fluorotetrahydro-1H-pyrrolizin-7a(5H)-yl)methoxy)-8,11-dimethyl-8,9,10,11-tetrahydro-7-oxa-1,3,6,11-tetraazacycloocta[de]naphthalen-5-yl)-4-(trifluoromethyl)aniline

To a solution of 5-chloro-4-fluoro-2-(((2R,7aS)-2-fluorotetrahydro-1H-pyrrolizin-7a(5H)-yl)methoxy)-8,11-dimethyl-8,9,10,11-tetrahydro-7-oxa-1,3,6,11-tetraazacycloocta[de]naphthalene (40 mg, 0.09 mmol) in dioxane/H₂O (10/2 mL) was added 3-chloro-5-(4,4,5,5-tetramethyl-1,3,2-dioxaborolan-2-yl)-4-(trifluoromethyl)aniline (43.8 mg, 0.136 mmol), NaHCO₃ (15.3 mg, 0.18 mmol) and Pd(dtbpf)Cl₂ (5.9 mg, 0.009 mmol) at room temperature, and the mixture was stirred at 100° C. for 3 hours. The resulting cooled mixture was concentrated and purified by column chromatography (DCM/MeOH=10/1) to give a residue which was further purified by Prep-HPLC to give the title product (1.58 mg). ¹H NMR (500 MHz, CD₃OD) δ6.92-6.85 (m, 1H), 6.62-6.37 (m, 1H), 5.43-5.28 (m, 1H), 4.61-4.49 (m, 2H), 4.43-4.23 (m, 2H), 3.83-3.73 (m, 1H), 3.57-3.48 (m, 1H), 3.41-3.39 (m, 3H), 3.13-3.05 (m, 1H), 2.42-1.84 (m, 8H), 1.48-1.42 (m, 3H). MS (ESI, m/e) [M+H]⁺ 599.2.

Example 15: 3-chloro-5-(4-fluoro-2-(((2R,7aS)-2-fluorotetrahydro-1H-pyrrolizin-7a(5H)-yl) methoxy)-9,11-dimethyl-8,9,10,11-tetrahydro-7-oxa-1,3,6,11 tetraazacycloocta[de]naphthalen-5-yl)-4-(trifluoromethyl)aniline

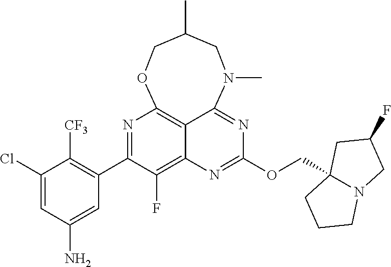 [SMILES: CC1COc2nc(-c3cc(N)cc(Cl)c3C(F)(F)F)c(F)c3nc(OC[C@@]45CCCN4C[C@H](F)C5)nc(c23)N(C)C1]

Step 1: 3-((5,7-dichloro-8-fluoro-2-(methylthio) pyrido[4,3-d]pyrimidin-4-yl) (methyl)amino)-2-methylpropan-1-ol

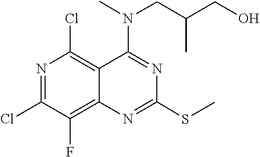 [SMILES: CSc1nc(N(C)CC(C)CO)c2c(Cl)nc(Cl)c(F)c2n1]

To a solution of 4,5,7-trichloro-8-fluoro-2-(methylthio) pyrido[4,3-d]pyrimidine (1000 mg, 3.3 mmol) in DCM (30 mL) was added DIPEA (1300 mg, 9.9 mmol) and 2-methyl-3-(methylamino) propan-1-ol (340 mg, 3.3 mmol) at 0° C. The resulting mixture was stirred at 0° C. to room temperature for 1 hour. After completion, the reaction mixture was concentrated to give the residue. The residue was purified by flash column silica (PE: EtOAc=1:1) to give the title product (610 mg). MS (ESI, m/e) [M+H]⁺ 365.1.

Step 2: 5-chloro-4-fluoro-9,11-dimethyl-2-(methylthio)-8,9,10,11-tetrahydro-7-oxa-1,3,6,11-tetraazacycloocta[de]naphthalene

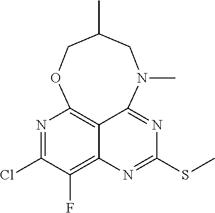 [SMILES: CSc1nc2c3c(nc(Cl)c(F)c3n1)OCC(C)CN2C]

To a solution of 3-((5,7-dichloro-8-fluoro-2-(methylthio) pyrido[4,3-d]pyrimidin-4-yl) (methyl)amino)-2-methylpropan-1-ol (585 mg, 1.6 mmol) in THF (25 mL) was added NaH (128 mg, 3.2 mmol) at room temperature, the mixture was stirred at r.t. for 2 hrs. Upon completion, the mixture was evaporated. The residue was purified by chromatography column on silica (PE: EtOAc=1:2) to give the title product (486 mg). MS (ESI, m/e) [M+H]⁺ 329.1.

Step 3: 5-chloro-4-fluoro-2-(((2R,7aS)-2-fluorotetrahydro-1H-pyrrolizin-7a(5H)-yl) methoxy)-9,11-dimethyl-8,9,10,11-tetrahydro-7-oxa-1,3,6,11-tetraazacycloocta[de]naphthalene

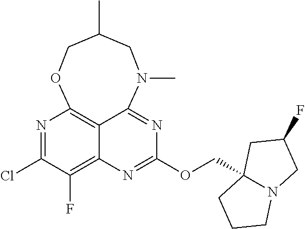 [SMILES: CC1COc2nc(Cl)c(F)c3nc(OC[C@@]45CCCN4C[C@H](F)C5)nc(c23)N(C)C1]

To a solution of 5-chloro-4-fluoro-9,11-dimethyl-2-(methylthio)-8,9,10,11-tetrahydro-7-oxa-1,3,6,11-tetraazacycloocta[de]naphthalene (486 mg, 1.48 mmol) in DCM (20 mL) was added m-CPBA (255 mg, 1.48 mmol) at rt., and the mixture was stirred at rt. for 1h as solution 1. Meanwhile, to a solution of ((2R,7aS)-2-fluorotetrahydro-1H-pyrrolizin-7a(5H)-yl) methanol (706 mg, 4.44 mmol) in 20 mL THF was added LiHMDS (1 N in THF, 3 mL, 3 mmol) at room temperature and it was stirred at room temperature for 1 hour as the solution 2. Then the solution 2 was added to solution 1 at room temperature and the mixture was stirred at room temperature for 2 hours. After completion, the reaction mixture was evaporated. The residue was purified by chromatography column on silica (DCM: MeOH=10:1) to give the title product (417 mg, crude). MS (ESI, m/e) [M+H]⁺ 439.1.

Step 4: 3-chloro-5-(4-fluoro-2-(((2R,7aS)-2-fluorotetrahydro-1H-pyrrolizin-7a(5H)-yl) methoxy)-9,11-dimethyl-8,9,10,11-tetrahydro-7-oxa-1,3,6,11-tetraazacycloocta[de]naphthalen-5-yl)-4-(trifluoromethyl)aniline

To a solution of 5-chloro-4-fluoro-2-(((2R,7aS)-2-fluorotetrahydro-1H-pyrrolizin-7a(5H)-yl) methoxy)-9,11-dimethyl-8,9,10,11-tetrahydro-7-oxa-1,3,6,11-tetraazacycloocta[de]naphthalene (50 mg, 0.11 mmol) in dioxane/H₂O(10/2 mL) was added 3-chloro-5-(4,4,5,5-tetramethyl-1,3,2-dioxaborolan-2-yl)-4-(trifluoromethyl) aniline (85 mg, 0.275 mmol), NaHCO₃ (28 mg, 0.33 mmol) and Pd(dtbpf)Cl₂ (7 mg, 0.011 mmol) at room temperature, and the mixture was stirred at 95° C. for 3 hours. The resulting cooled mixture was concentrated and purified by column chromatography (DCM/MeOH=10/1) to give a residue which was further purified by Prep-HPLC to give the title product (1.66 mg). ¹H NMR (500 MHZ, CD₃OD) δ6.89-6.85 (m, 1H), 6.54-6.44 (m, 1H), 5.43-5.26 (m, 1H), 4.43-4.21 (m, 4H), 3.58-3.32 (m, 8H), 3.12-3.05 (m, 1H), 2.43-1.91 (m, 7H), 1.25-1,17 (m, 3H). MS (ESI, m/e) [M+H]⁺ 599.2.

Example 16: 5-ethyl-6-fluoro-4-(4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalen-5-yl)naphthalen-2-ol

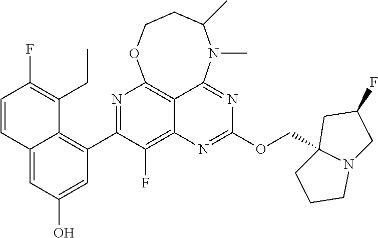 [SMILES: CCc1c(F)ccc2cc(O)cc(-c3nc4c5c(nc(OC[C@@]67CCCN6C[C@H](F)C7)nc5c3F)N(C)C(C)CCO4)c12]

Step 1: 5-(8-ethyl-7-fluoro-3-(methoxymethoxy) naphthalen-1-yl)-4-fluoro-2-(((2R,7aS)-2-fluorotetrahydro-1H-pyrrolizin-7a(5H)-yl) methoxy)-10,11-dimethyl-8,9,10,11-tetrahydro-7-oxa-1,3,6,11-tetraazacycloocta[de]naphthalene

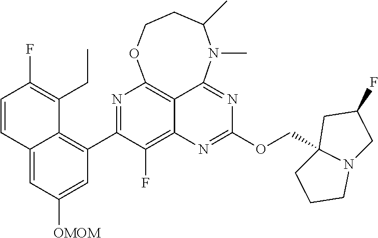

To a solution of 5-chloro-4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalene (48 mg, 0.11 mmol) in dioxane/H₂O (10/2 mL) was added 2-(8-ethyl-7-fluoro-3-(methoxymethoxy) naphthalen-1-yl)-4,4,5,5-tetramethyl-1,3,2-dioxaborolane (79 mg, 0.22 mmol), NaHCO₃(28 mg, 0.33 mmol) and Pd(dtbpf)Cl₂ (7 mg, 0.011 mmol) at room temperature, and the mixture was stirred at 95° C. for 3 hours. The resulting cooled mixture was concentrated and purified by column chromatography (DCM/MeOH=10/1) to give the title product (36 mg). MS (ESI, m/e) [M+H]⁺ 638.2.

Step 2: 5-ethyl-6-fluoro-4-(4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalen-5-yl)naphthalen-2-ol

To a solution of 5-(8-ethyl-7-fluoro-3-(methoxymethoxy) naphthalen-1-yl)-4-fluoro-2-(((2R,7aS)-2-fluorotetrahydro-1H-pyrrolizin-7a(5H)-yl) methoxy)-10,11-dimethyl-8,9,10,11-tetrahydro-7-oxa-1,3,6,11-tetraazacycloocta[de]naphthalene (36 mg, 0.056 mmol) in EtOAc (4 mL) was added HCl (4N in dioxane, 1 mL) at room temperature, and the mixture was stirred at r.t. for 2 hrs. The resulting mixture was concentrated at room temperature and pH was adjusted to 7 with Na₂CO₃, and the organic layer was concentrated to give a residue which was further purified by Prep-HPLC to give title product (8.8 mg). ¹H NMR (500 MHZ, CD₃OD) δ7.66-7.63 (m, 1H), 7.30-6.96 (m, 3H), 5.47-5.28 (m, 1H), 4.58-4.00 (m, 5H), 3.61-3.35 (m, 3H), 3.32-3.28 (m, 3H), 3.16-3.12 (m, 1H), 2.61-1.96 (m, 8H), 1.48-1.42 (m, 3H), 0.91-0.85 (m, 3H). MS (ESI, m/e) [M+H]⁺ 594.3.

Example 17: 3-chloro-5-(4-fluoro-2-(((2R,7aS)-2-fluorotetrahydro-1H-pyrrolizin-7a(5H)-yl)methoxy)-11-(2-(methylamino)ethyl)-8,9,10,11-tetrahydro-7-oxa-1,3,6,11-tetraazacycloocta[de]naphthalen-5-yl)-4-(trifluoromethyl)aniline

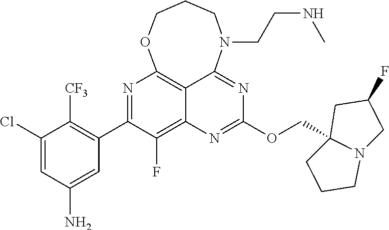 [SMILES: CNCCN1CCCOc2nc(-c3cc(N)cc(Cl)c3C(F)(F)F)c(F)c3nc(OC[C@@]45CCCN4C[C@H](F)C5)nc1c23]

Step 1: tert-butyl (2-((3-hydroxypropyl)amino)ethyl)(methyl)carbamate

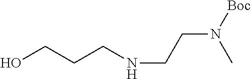 [SMILES: CN(CCNCCCO)C(=O)OC(C)(C)C]

The solution of 3-bromopropan-1-ol (2.1 g, 10 mmol) in tert-butyl (2-aminoethyl)(methyl)carbamate (3 mL) was stirred at 40° C. for 15 hrs. After completion, the reaction mixture was concentrated to give the residue. The residue was used directly in the next step without more purification (4.3 g, crude). MS (ESI, m/e) [M+H]⁺ 233.5.

Step 2: tert-butyl (2-((5,7-dichloro-8-fluoro-2-(methylthio)pyrido[4,3-d]pyrimidin-4-yl)(3-hydroxypropyl)amino)ethyl)(methyl)carbamate

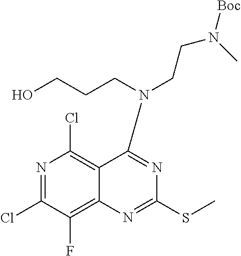 [SMILES: CSc1nc(N(CCCO)CCN(C)C(=O)OC(C)(C)C)c2c(Cl)nc(Cl)c(F)c2n1]

The mixture of 4,5,7-trichloro-8-fluoro-2-(methylthio)pyrido[4,3-d]pyrimidine (2.1 g, 7.17 mmol), tert-butyl (2-((3-hydroxypropyl)amino)ethyl)(methyl)carbamate (3.9 g, 21.5 mmol) and DIPEA (4.6 g, 36 mmol) in DCM (20 mL) was stirred at 25° C. for 2 hrs. After completion, the reaction mixture was diluted with DCM (50 mL), and washed with water (15 mL×3). The organic layer was dried over anhydrous Na₂SO₄, filtered and the filtrate was concentrated to give the residue. The residue was purified by silica gel (PE: EtOAc=1:4) to give the title product (800 mg). MS (ESI, m/e) [M+H]⁺ 494.5.

Step 3: tert-butyl (2-(5-chloro-4-fluoro-2-(methylthio)-9,10-dihydro-7-oxa-1,3,6,11-tetraazacycloocta[de]naphthalen-11(8H)-yl)ethyl)(methyl)carbamate

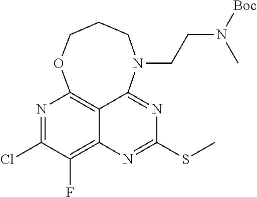 [SMILES: CSc1nc2c3c(nc(Cl)c(F)c3n1)OCCCN2CCN(C)C(=O)OC(C)(C)C]

To a solution of tert-butyl (2-((5,7-dichloro-8-fluoro-2-(methylthio)pyrido[4,3-d]pyrimidin-4-yl)(3-hydroxypropyl)amino) ethyl) (methyl)carbamate (494 mg, 1.0 mmol) in THF (5 mL) was added LiHMDS (2 mL, 2 mmol) at 0° C. The resulting mixture was stirred at room temperature for 4 hrs. After completion, the reaction mixture was concentrated to give the residue. The residue was purified by Prep-TLC (PE: EtOAc=1:1) to give the title product (380 mg). MS (ESI, m/e) [M+H]⁺ 458.4.

Step 4: tert-butyl (2-(5-chloro-4-fluoro-2-(((2R,7aS)-2-fluorotetrahydro-1H-pyrrolizin-7a(5H)-yl)methoxy)-9,10-dihydro-7-oxa-1,3,6,11-tetraazacycloocta[de]naphthalen-11(8H)-yl)ethyl)(methyl)carbamate

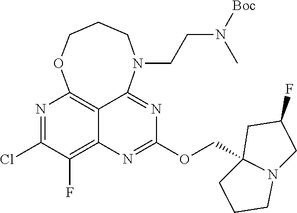 [SMILES: CN(CCN1CCCOc2nc(Cl)c(F)c3nc(OC[C@@]45CCCN4C[C@H](F)C5)nc1c23)C(=O)OC(C)(C)C]

A mixture of tert-butyl (2-(5-chloro-4-fluoro-2-(methylthio)-9,10-dihydro-7-oxa-1,3,6,11-tetraazacycloocta[de]naphthalen-11(8H)-yl)ethyl)(methyl)carbamate (535 mg, 1,17 mmol), and m-CPBA (404 mg, 2.34 mmol) in DCM (10 mL) was stirred at 25° C. for 1 h to give mixture 1. Meanwhile, to a solution of ((2R,7aS)-2-fluorotetrahydro-1H-pyrrolizin-7a(5H)-yl)methanol (80 mg, 0.5 mmol) in THF (5 mL) was added LiHMDS (0.5 ml, 0.5 mmol) at 0° C., and the resulting mixture was stirred at 0° C. for 0.5 h to give mixture 2. Then, to mixture 2 was added mixture 1 and the resulting mixture was stirred at room temperature for 2 hrs. After completion. The reaction mixture was concentrated to give the residue. The residue was purified by Prep-TLC (PE: EtOAc=1:10) to give the title product (98 mg). MS (ESI, m/e) [M+H]⁺ 569.5.

Step 5: tert-butyl (2-(5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-9,10-dihydro-7-oxa-1,3,6,11-tetraazacycloocta[de]naphthalen-11(8H)-yl)ethyl)(methyl)carbamate

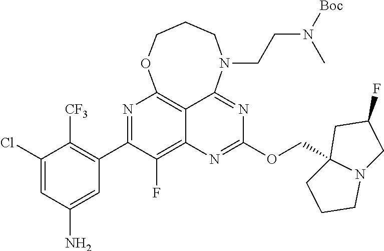 [SMILES: CN(CCN1CCCOc2nc(-c3cc(N)cc(Cl)c3C(F)(F)F)c(F)c3nc(OC[C@@]45CCCN4C[C@H](F)C5)nc1c23)C(=O)OC(C)(C)C]

A mixture of tert-butyl (2-(5-chloro-4-fluoro-2-(((2R,7aS)-2-fluorotetrahydro-1H-pyrrolizin-7a(5H)-yl)methoxy)-9,10-dihydro-7-oxa-1,3,6,11-tetraazacycloocta[de]naphthalen-11(8H)-yl)ethyl)(methyl)carbamate (85 mg, 0.15 mmol), 3-chloro-5-(4,4,5,5-tetramethyl-1,3,2-dioxaborolan-2-yl)-4-(trifluoromethyl)aniline (145 mg, 0.45 mmol), Pd(dtbpf)Cl₂ (10 mg, 0.015 mmol), and NaHCO₃(38 mg, 0.45 mmol) in dioxane (10 mL) and water (2 mL) was stirred at 80° C. for 2 hrs. After completion, the reaction mixture was concentrated, which was purified by prep-TLC (EtOAc: MeOH=10:1) to give the titled product (90 mg). MS (ESI, m/e) [M+H]⁺ 728.5.

Step 6: 3-chloro-5-(4-fluoro-2-(((2R,7aS)-2-fluorotetrahydro-1H-pyrrolizin-7a(5H)-yl)methoxy)-11-(2-(methylamino)ethyl)-8,9,10,11-tetrahydro-7-oxa-1,3,6,11-tetraazacycloocta[de]naphthalen-5-yl)-4-(trifluoromethyl)aniline

To a solution of tert-butyl (2-(5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-9,10-dihydro-7-oxa-1,3,6,11-tetraazacycloocta[de]naphthalen-11(8H)-yl)ethyl)(methyl)carbamate (80 mg, 0.11 mmol) in DCM (4 mL) was added TFA (4 mL) at room temperature. The resulting mixture was stirred at room temperature for 2 hrs. After completion, the reaction mixture was concentrated to give the residue. The residue was purified by prep-HPLC to give the title product (17.0 mg). ¹H NMR (500 MHZ, CD₃OD) δ6.89 (s, 1H), 6.50 (s. 1H), 5.48-5.30 (m, 1H), 4.55-4.23 (m, 5H), 4.08-3.95 (m, 1H), 3.72-3.65 (m, 2H), 3.59-3.44 (m, 3H), 3.38-3.35 (m, 2H), 3.25-3.16 (m, 1H), 2.77 (s, 3H), 2.45-1.93 (m, 8H). MS (ESI, m e) [M+H]⁺ 628.3.

Example 18: 2-amino-4-(8′-chloro-10′-fluoro-2′-(((2R,7aS)-2-fluorotetrahydro-1H-pyrrolizin-7a(5H)-yl)methoxy)-4′-methyl-4′H, 6′H-spiro[oxetane-3,5′-[1,4]oxazepino[5,6,7-de]quinazolin]-9′-yl)-7-fluorobenzo[b]thiophene-3-carbonitrile

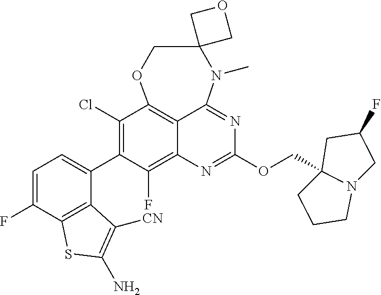 [SMILES: CN1c2nc(OC[C@@]34CCCN3C[C@H](F)C4)nc3c(F)c(-c4ccc(F)c5sc(N)c(C#N)c45)c(Cl)c(c23)OCC12COC2]

Example 18 was prepared by a procedure similar to that described in Example 1 by replacing 7-bromo-4-chloro-5,8-difluoro-2-(methylthio)quinazoline and 3-(methylamino)propan-1-ol with 7-bromo-4,6-dichloro-5,8-difluoro-2-(methylthio)quinazoline and (3-(methylamino)oxetan-3-yl)methanol to give the title product (0.8 mg). ¹H NMR (500 MHZ, DMSO-d₆) δ 8.09 (s, 2H), 7.28-7.19 (m, 1H), 7.19-7.09 (m, 1H), 5.39-5.28 (m, 1H), 5.05-4.97 (m, 1H), 4.97-4.82 (m, 3H), 4.65-4.50 (m, 2H), 4.20-3.98 (m, 2H), 3.48 (s, 3H), 3.16-3.00 (m, 2H), 2.92-2.79 (m, 1H), 2.17-1.95 (m, 1H), 1.93-1.75 (m, 2H), 1.51-1.41 (m, 1H), 1.34-1.26 (m, 1H), 0.89-0.82 (m, 2H). MS (ESI, m/e) [M+H]⁺ 643.3.

Example 19: 2-amino-4-((R)-9-chloro-11-fluoro-2-(((2R,7aS)-2-fluorotetrahydro-1H-pyrrolizin-7a(5H)-yl)methoxy)-4,5-dimethyl-4,5,6,7-tetrahydro-[1,5]oxazocino[4,3,2-de]quinazolin-10-yl)-7-fluorobenzo[b]thiophene-3-carbonitrile

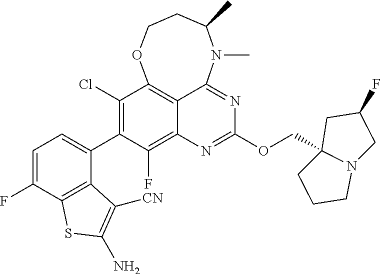 [SMILES: C[C@@H]1CCOc2c(Cl)c(-c3ccc(F)c4sc(N)c(C#N)c34)c(F)c3nc(OC[C@@]45CCCN4C[C@H](F)C5)nc(c23)N1C]

Example 19 was prepared by a procedure similar to that described in Example 1 by replacing 7-bromo-4-chloro-5,8-difluoro-2-(methylthio)quinazoline and 3-(methylamino)propan-1-ol with 7-bromo-4,6-dichloro-5,8-difluoro-2-(methylthio)quinazoline and (R)-3-(methylamino)butan-1-ol to give the title product (5.8 mg). ¹H NMR (500 MHZ, DMSO-d₆) δ 8.06 (s, 2H), 7.35-7.05 (m, 2H), 5.42-5.20 (m, 1H), 4.49-4.17 (m, 2H), 4.12-4.04 (m, 1H), 4.00-3.96 (m, 1H), 3.90-3.87 (m, 1H), 3.33-3.29 (m, 1H), 3.19-3.06 (m, 5H), 2.88-2.76 (m, 1H), 2.18-2.05 (m, 4H), 2.05-1.71 (m, 4H), 1.35-1.20 (m, 3H). MS (ESI, m/e) [M+H]⁺ 629.4.

Example 20: 3-chloro-5-(4-fluoro-2-(((2R,7aS)-2-fluorotetrahydro-1H-pyrrolizin-7a(5H)-yl)methoxy)-10-methyl-11-(2-(methylamino)ethyl)-8,9,10,11-tetrahydro-7-oxa-1,3,6,11-tetraazacycloocta[de]naphthalen-5-yl)-4-(trifluoromethyl)aniline

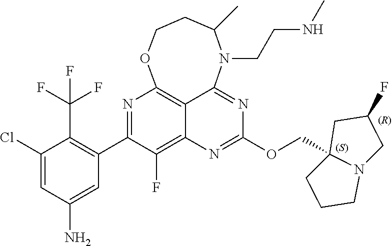

Step 1: 4-((tert-butyldimethylsilyl)oxy)butan-2-one

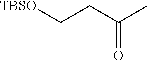

To a solution of 4-hydroxybutan-2-one (5 g, 56.82 mmol), DIPEA (20 mL, 113.64 mmol) and DMAP (685 mg, 5.62 mmol) in dichloromethane (200 mL) was added TBSOTf (16.5 g, 62.49 mmol) at 0° C. The mixture was stirred at room temperature for 2 hrs. Then it was diluted with dichloromethane and water. The organic layer was combined, dried over sodium sulfate and evaporated. The residue was purified by chromatography column on silica gel (Petroleum ether: EtOAc=5:1) to give the title compound (9.1 g). MS (ESI, m/e) [M+H]⁺ 203.5.

Step 2: tert-butyl (2-((4-((tert-butyldimethylsilyl)oxy)butan-2-yl)amino)ethyl)(methyl)carbamate

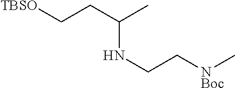 [SMILES: CC(CCO[Si](C)(C)C(C)(C)C)NCCN(C)C(=O)OC(C)(C)C]

To a solution of 4-((tert-butyldimethylsilyl)oxy)butan-2-one (1 g, 4.95 mmol) and tert-butyl (2-aminoethyl)(methyl)carbamate (861 mg, 4.95 mmol) in MeOH (100 mL) was added sodium triacetoxyborohydride (1.3 g, 5.94 mmol) at rt. The mixture was stirred at rt for 2 hrs. The solvent was evaporated in vacuo. The residue was purified by chromatography column on silica gel (DCM:MeOH=20:1) to give the title compound (320 mg). MS (ESI, me) [M+H]⁺ 361.5.

Step 3: tert-butyl (2-((4-hydroxybutan-2-yl)amino)ethyl)(methyl)carbamate

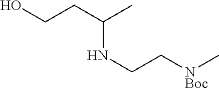 [SMILES: CC(CCO)NCCN(C)C(=O)OC(C)(C)C]

To a solution of tert-butyl (2-((4-((tert-butyldimethylsilyl)oxy)butan-2-yl)amino)ethyl)(methyl)carbamate (300 mg, 0.83 mmol) in THF (20 mL) was added TBAF (0.92 mL, 0.92 mmol, 1M THF solution) at rt. The mixture was stirred at rt overnight. The solvent was evaporated in vacuo. The residue was purified by chromatography column on silica gel (DCM:MeOH=10:1) to give crude product (150 mg contain TBAF). MS (ESI, m/e) [M+1]⁺247.5.

Step 4: tert-butyl (2-((5,7-dichloro-8-fluoro-2-(methylthio)pyrido[4,3-d]pyrimidin-4-yl)(4-hydroxybutan-2-yl)amino)ethyl)(methyl)carbamate

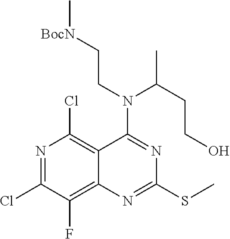 [SMILES: CSc1nc(N(CCN(C)C(=O)OC(C)(C)C)C(C)CCO)c2c(Cl)nc(Cl)c(F)c2n1]

To a stirred solution of 4,5,7-trichloro-8-fluoro-2-(methylthio)pyrido[4,3-d]pyrimidine (1.78 mmol) in DCM (10 mL) was added DIEA (689 mg, 5.34 mmol) and tert-butyl (2-((4-hydroxybutan-2-yl)amino)ethyl)(methyl)carbamate (440 mg, 1.78 mmol) at 0° C. The resulting mixture was stirred for 30 mins at room temperature. The reaction mixture was concentrated and purified by flash chromatography (Petroleum ether: EtOAc=4:1 to 1:2) to afford the desired product (262 mg). MS (ESI, m/e) [M+H]⁺ 508.5.

Step 5: tert-butyl (2-(5-chloro-4-fluoro-10-methyl-2-(methylthio)-9,10-dihydro-7-oxa-1,3,6,11-tetraazacycloocta[de]naphthalen-11(8H)- yl)ethyl)(methyl)carbamate

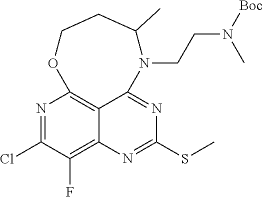 [SMILES: CSc1nc2c3c(nc(Cl)c(F)c3n1)OCCC(C)N2CCN(C)C(=O)OC(C)(C)C]

To a stirred solution of tert-butyl (2-((5,7-dichloro-8-fluoro-2-(methylthio)pyrido[4,3-d]pyrimidin-4-yl)(4-hydroxybutan-2-yl)amino)ethyl)(methyl)carbamate (262 mg, 0.52 mmol) in THF (10 mL) was added NaH (31 mg, 0.77 mmol, 60%) at 0° C. The resulting mixture was stirred at room temperature for 2 h. The reaction was quenched with H₂O, then extracted with EtOAc. The organic layer was washed with brine, dried over anhydrous Na₂SO₄, concentrated in vacuo. The residue was purified by flash chromatography (PE/EtOAc=10:1 to 1:1) to afford the product (162 mg). MS (ESI, m/e) [M+H]⁺ 472.5.

Step 6: tert-butyl (2-(5-chloro-4-fluoro-10-methyl-2-(methylsulfinyl)-9,10-dihydro-7-oxa-1,3,6,11-tetraazacycloocta[de]naphthalen-11(8H)-yl)ethyl)(methyl)carbamate

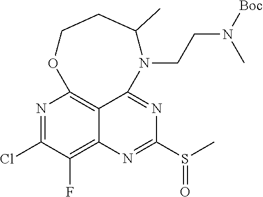 [SMILES: CC1CCOc2nc(Cl)c(F)c3nc(S(C)=O)nc(c23)N1CCN(C)C(=O)OC(C)(C)C]

To a solution of 5-chloro-4-fluoro-11-methyl-2-(methylthio)-8,9,10,11-tetrahydro-7-oxa-1,3,6,11-tetraazacycloocta[de]naphthalene (162 mg, 0.34 mmol) in DCM (6 mL) was added m-CPBA (84 mg, 0.41 mmol, 85%) at 0° C., the resulting mixture was stirred at 0° C. for 10 min. The reaction was quenched with saturated Na₂S₂O₃ aqueous solution, then extracted with DCM. The organic layer was washed with saturated NaHCO₃ aqueous solution and brine, dried over anhydrous Na₂SO₄, concentrated in vacuo. The residue was directly used in the next step without further purification. MS (ESI, m/e) [M+H]⁺ 488.5.

Step 7: tert-butyl (2-(5-chloro-4-fluoro-2-(((2R,7aS)-2-fluorotetrahydro-1H-pyrrolizin-7a(5H)-yl)methoxy)-10-methyl-9,10-dihydro-7-oxa-1,3,6,11-tetraazacycloocta[de]naphthalen-11(8H)-yl)ethyl)(methyl)carbamate

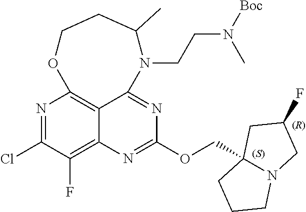

To a solution of tert-butyl (2-(5-chloro-4-fluoro-10-methyl-2-(methylsulfinyl)-9,10-dihydro-7-oxa-1,3,6,11-tetraazacycloocta[de]naphthalen-11(8H)-yl)ethyl)(methyl)carbamate (0.34 mmol) and ((2R,7aS)-2-fluorotetrahydro-1H-pyrrolizin-7a(5H)-yl)methanol (108 mg, 0.68 mmol) in THF (6 mL) was added LiHMDS (0.68 mL, 0.68 mmol, 1M in THF) dropwise at 0° C., the resulting mixture was stirred at 0° C. for 20 min. The reaction was quenched with saturated NH₄Cl aqueous solution, then extracted with EtOAc. The organic layer was washed with brine, dried over anhydrous Na₂SO₄, concentrated in vacuo. The residue was purified by flash chromatography (DCM/MeOH=100:1 to 30:1) to afford the product (63 mg). MS (ESI, m/e) [M+H]⁺ 583.5.

Step 8: tert-butyl (2-(5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-10-methyl-9,10-dihydro-7-oxa-1,3,6,11-tetraazacycloocta[de]naphthalen-11(8H)-yl)ethyl)(methyl)carbamate

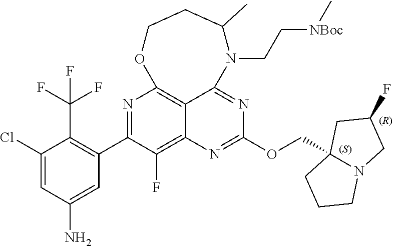

To a mixture of tert-butyl (2-(5-chloro-4-fluoro-2-(((2R,7aS)-2-fluorotetrahydro-1H-pyrrolizin-7a(5H)-yl)methoxy)-10-methyl-9,10-dihydro-7-oxa-1,3,6,11-tetraazacycloocta[de]naphthalen-11(8H)-yl)ethyl) (methyl)carbamate (60 mg, 0.10 mmol), 3-chloro-5-(4,4,5,5-tetramethyl-1,3,2-dioxaborolan-2-yl)-4-(trifluoromethyl)aniline (64 mg, 0.2 mmol), Pd(dtbpf)Cl₂ (7.8 mg, 0.012 mmol) and NaHCO₃ (15 mg, 0.18 mmol) was added dioxane (2.0 mL) and H₂O (0.4 mL). The reaction mixture was stirred at 90° C. for 2 hrs. The reaction mixture was concentrated and purified by flash chromatography (DCM/MeOH=100:1 to 5:1), then prep-HPLC to give the title product (30 mg).

Step 9: 3-chloro-5-(4-fluoro-2-(((2R,7aS)-2-fluorotetrahydro-1H-pyrrolizin-7a(5H)-yl)methoxy)-10-methyl-11-(2-(methylamino)ethyl)-8,9,10,11-tetrahydro-7-oxa-1,3,6,11-tetraazacycloocta[de]naphthalen-5-yl)-4-(trifluoromethyl)aniline

To a mixture of tert-butyl (2-(5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-10-methyl-9,10-dihydro-7-oxa-1,3,6,11-tetraazacycloocta[de]naphthalen-11(8H)-yl)ethyl)(methyl)carbamate (30 mg, 0.04 mmol) in DCM (5 mL) was added TFA (1 mL). The reaction mixture was stirred at room temperature for 2 hrs. The reaction mixture was concentrated and purified by prep-HPLC to give the formate of title product (4 mg). ¹H NMR (500 MHZ, CD₃OD) δ 8.48 (s, 1H), 6.89-6.88 (m, 1H), 6.59-6.35 (m, 1H), 5.42-5.31 (m, 1H), 4.52-4.11 (m, 6H), 3.85-3.74 (m, 1H), 3.44-3.34 (m, 5H), 3.21-3.11 (m, 1H), 2.81 (s, 3H), 2.31-1.98 (m, 8H), 1.51-1.44 (m, 3H). MS (ESI, m/e) [M+H]⁺ 642.5.

Example 21: 3-chloro-5-(4-fluoro-2-(((2R,7aS)-2-fluorotetrahydro-1H-pyrrolizin-7a(5H)-yl)methoxy)-8,10,11-trimethyl-8,9,10,11-tetrahydro-7-oxa-1,3,6,11-tetraazacycloocta[de]naphthalen-5-yl)-4-(trifluoromethyl)aniline

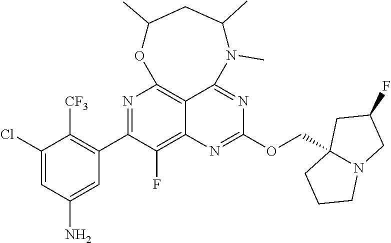 [SMILES: CC1CC(C)N(C)c2nc(OC[C@@]34CCCN3C[C@H](F)C4)nc3c(F)c(-c4cc(N)cc(Cl)c4C(F)(F)F)nc(c23)O1]

Step 1: 7-chloro-8-fluoro-5-((4-(methylamino)pentan-2-yl)oxy)-2-(methylthio)pyrido[4,3-d]pyrimidin-4-ol

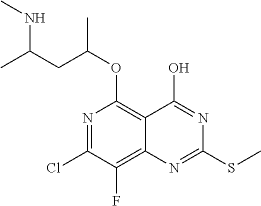 [SMILES: CNC(C)CC(C)Oc1nc(Cl)c(F)c2nc(SC)nc(O)c12]

To a solution of 4-(methylamino)pentan-2-ol (234 mg, 2 mmol) in THF (10 mL) was added sodium hydride (80 mg, 2 mmol) at room temperature. The resulting mixture was stirred at room temperature for 1 hour. Then, 5,7-dichloro-8-fluoro-2-(methylthio)pyrido[4,3-d]pyrimidin-4-ol (560 mg, 2 mmol) was added to the reaction mixture and stirred at room temperature for 1 hour. After completion, the reaction mixture was concentrated to give the residue. The residue was purified by flash column silica (DCM: MeOH=10:1) to give the title product (260 mg). MS (ESI, m/e) [M+H]⁺ 361,1.

Step 2: 5-chloro-4-fluoro-8,10,11-trimethyl-2-(methylthio)-8,9,10,11-tetrahydro-7-oxa-1,3,6,11-tetraazacycloocta[de]naphthalene

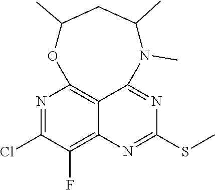 [SMILES: CSc1nc2c3c(nc(Cl)c(F)c3n1)OC(C)CC(C)N2C]

To a solution of 7-chloro-8-fluoro-5-((4-(methylamino)pentan-2-yl)oxy)-2-(methylthio)pyrido[4,3-d]pyrimidin-4-ol (260 mg, 0.72 mmol) in 20 mL N,N-Dimethylformamide was added N,N-Diisopropylethylamine (278.7 mg, 2.16 mmol) and 2-(7-Azabenzotriazol-1-yl)-N,N,N′,N′-tetramethyluronium hexafluorophosphate (547.2 mg, 1.44 mmol) at room temperature and it was stirred at room temperature for 1 h. Then, more of N,N-Diisopropylethylamine (278.7 mg, 2.16 mmol) and 2-(7-Azabenzotriazol-1-yl)-N,N,N′,N′-tetramethyluronium hexafluorophosphate (547.2 mg, 1.44 mmol) was added and the mixture was stirred at room temperature for 2 hours. Then, the mixture was diluted with water, extracted with EtOAc, the organic layer was combined, dried over sodium sulfate and evaporated. The residue was purified by chromatography column on silica (PE: EtOAc=1:5) to give the title product (70 mg). MS (ESI, m/e) [M+H]⁺ 343.1.

Step 3: 5-chloro-4-fluoro-2-(((2R,7aS)-2-fluorotetrahydro-1H-pyrrolizin-7a(5H)-yl)methoxy)-8,10,11-trimethyl-8,9,10,11-tetrahydro-7-oxa-1,3,6,11-tetraazacycloocta[de]naphthalene

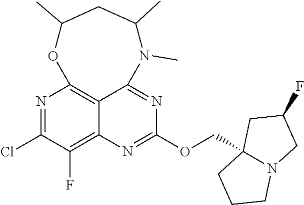 [SMILES: CC1CC(C)N(C)c2nc(OC[C@@]34CCCN3C[C@H](F)C4)nc3c(F)c(Cl)nc(c23)O1]

To a solution of 5-chloro-4-fluoro-8,10,11-trimethyl-2-(methylthio)-8,9,10,11-tetrahydro-7-oxa-1,3,6,11-tetraazacycloocta[de]naphthalene (70 mg 0.205 mmol) in 10 mL dichloromethane was added 3-Chloroperoxybenzoic acid (42.4 mg 0.246 mmol) at room temperature and it was stirred at room temperature for 2 hours as the mixture 1. Meanwhile, LiHMDS (1 N in THF, 0.4 mL) was added to ((2R,7aS)-2-fluorotetrahydro-1H-pyrrolizin-7a(5H)-yl)methanol (97.6 mg 0.614 mmol) in THF (10 mL) at room temperature and it was stirred at room temperature for 2 hours as the mixture 2. Then, the mixture 2 was added to mixture 1 at room temperature and the mixture was stirred at room temperature for 2 hours. Then it was evaporated. The residue was purified by chromatography column on silica (DCM: MeOH=10:1) to give the title product (87 mg). MS (ESI, m/e) [M+H]⁺ 454.2.

Step 4: 3-chloro-5-(4-fluoro-2-(((2R,7aS)-2-fluorotetrahydro-1H-pyrrolizin-7a(5H)-yl)methoxy)-8,10,11-trimethyl-8,9,10,11-tetrahydro-7-oxa-1,3,6,11-tetraazacycloocta[de]naphthalen-5-yl)-4-(trifluoromethyl)aniline

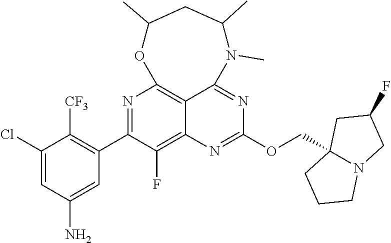 [SMILES: CC1CC(C)N(C)c2nc(OC[C@@]34CCCN3C[C@H](F)C4)nc3c(F)c(-c4cc(N)cc(Cl)c4C(F)(F)F)nc(c23)O1]

To a mixture of 5-chloro-4-fluoro-2-(((2R,7aS)-2-fluorotetrahydro-1H-pyrrolizin-7a(5H)-yl)methoxy)-8,10,11-trimethyl-8,9,10,11-tetrahydro-7-oxa-1,3,6,11-tetraazacycloocta[de]naphthalene (87 mg, 0.192 mmol), 3-chloro-5-(4,4,5,5-tetramethyl-1,3,2-dioxaborolan-2-yl)-4-(trifluoromethyl)aniline (124 mg, 0.384 mmol), NaHCO₃(48.4 mg, 0.576 mmol) and Pd(dtbpf)Cl₂ (25 mg, 0.0384 mmol) was added dioxane (10 mL) and water (2 mL). The mixture was stirred at 95° C. for 3 hours. The resulting cooled mixture was concentrated and purified by column chromatography (DCM/MeOH=10/1) to give a residue which was further purified by Prep-HPLC to give the title product (1.6 mg). ¹H NMR (500 MHZ, DMSO-d₆) δ 6.95-6.80 (m, 1H), 6.63-6.39 (m, 1H), 6.36-6.27 (m, 2H), 5.39-5.21 (m, 1H), 4.57-4.40 (m, 1H), 4.15-4.06 (m, 1H), 4.03-3.96 (m, 1H), 3.61-3.47 (m, 1H), 3.13-3.06 (m, 5H), 3.02-3.01 (m, 1H), 2.85-2.79 (m, 1H), 2.12-1.96 (m, 4H), 1.86-1.69 (m, 4H), 1.43-1.37 (m, 3H), 1.26-1.20 (m, 3H). MS (ESI, m/e) [M+H]⁺ 613.2.

Example 22: 3-chloro-5-(4-fluoro-2-(((2R,7aS)-2-fluorotetrahydro-1H-pyrrolizin-7a(5H)-yl)methoxy)-9,10-dimethyl-9,10-dihydro-8H-7-oxa-1,3,6,10-tetraazacyclohepta[de]naphthalen-5-yl)-4-(trifluoromethyl)aniline

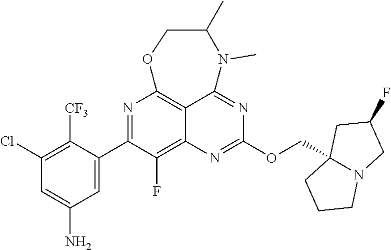 [SMILES: CC1COc2nc(-c3cc(N)cc(Cl)c3C(F)(F)F)c(F)c3nc(OC[C@@]45CCCN4C[C@H](F)C5)nc(c23)N1C]

Example 22 was prepared by a procedure similar to that described in Example 15 by replacing 2-methyl-3-(methylamino) propan-1-ol with 2-(methylamino)propan-1-ol to give the title product (4 mg). ¹H NMR (500 MHz, DMSO-d₆) δ 6.85 (s, 1H), 6.54-6.40 (m, 1H), 6.29 (s, 2H), 5.36-5.20 (m, 1H), 4.55-4.40 (m, 2H), 4.13-3.99 (m, 3H), 3.33-3.29 (m, 2H), 3.14-3.01 (m, 3H), 2.85-2.76 (m, 1H), 2.14-1.96 (m, 3H), 1.88-1.76 (m, 3H), 1.32-1.22 (m, 4H). MS (ESI, m/e) [M+H]⁺ 585.4.

Example 23: 3-chloro-5-(4-fluoro-2-(((2R,7aS)-2-fluorotetrahydro-1H-pyrrolizin-7a(5H)-yl)methoxy)-10-methyl-8H, 10H-7-oxa-1,3,6,10-tetraazaspiro[cyclohepta[de]naphthalene-9,3′-oxetan]-1(10a), 2,3a, 3a¹(6a), 5-pentaen-5-yl)-4-(trifluoromethyl)aniline

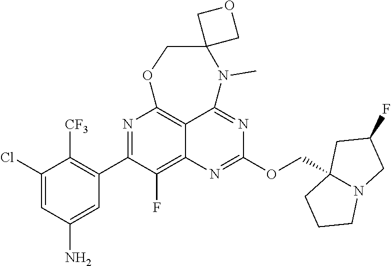 [SMILES: CN1c2nc(OC[C@@]34CCCN3C[C@H](F)C4)nc3c(F)c(-c4cc(N)cc(Cl)c4C(F)(F)F)nc(c23)OCC12COC2]

Step 1: 7-chloro-8-fluoro-5-((3-(methylamino) oxetan-3-yl) methoxy)-2-(methylthio) pyrido[4,3-d]pyrimidin-4-ol

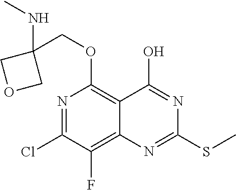 [SMILES: CNC1(COc2nc(Cl)c(F)c3nc(SC)nc(O)c23)COC1]

To a solution of (3-(methylamino) oxetan-3-yl) methanol (412 mg, 4 mmol) in THF (20 mL) was added sodium hydride (100 mg, 5 mmol) at room temperature. The resulting mixture was stirred at room temperature for 1 hour. Then, 5,7-dichloro-8-fluoro-2-(methylthio) pyrido[4,3-d]pyrimidin-4-ol (558 mg, 2 mmol) in THF (20 mL) was added to the reaction mixture and stirred at room temperature for 1 hour. After completion, the reaction mixture was concentrated to give the residue. The residue was purified by flash column silica (DCM: MeOH=10:1) to give the title product (826 mg crude). MS (ESI, m/e) [M+H]⁺ 361,1.

Step 2: 5-chloro-4-fluoro-10-methyl-2-(methylthio)-8H, 10H-7-oxa-1,3,6,10-tetraazaspiro[cyclohepta[de]naphthalene-9,3′-oxetan]-1(10a), 2,3a, 3 al(6a), 5-pentaene

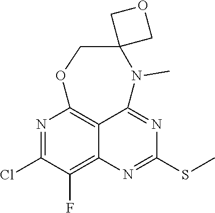 [SMILES: CSc1nc2c3c(nc(Cl)c(F)c3n1)OCC1(COC1)N2C]

To a solution of 7-chloro-8-fluoro-5-((3-(methylamino) oxetan-3-yl) methoxy)-2-(methylthio) pyrido[4,3-d]pyrimidin-4-ol (390 mg, 1.08 mmol) in 30 mL N,N-Dimethylformamide was added N,N-Diisopropylethylamine (774 mg, 6 mmol) and 2-(7-Azabenzotriazol-1-yl)-N,N,N′,N′-tetramethyluronium hexafluorophosphate (1100 mg, 3 mmol) at room temperature and it was stirred at room temperature for 1 h. Then it was diluted with water, extracted with EtOAc, the organic layer was combined, dried over sodium sulfate and evaporated. The residue was purified by chromatography column on silica (PE: EtOAc=1:5) to give the title product (457 mg crude). MS (ESI, m/e) [M+H]⁺ 343.1.

Step 3: 5-chloro-4-fluoro-2-(((2R,7aS)-2-fluorotetrahydro-1H-pyrrolizin-7a(5H)-yl) methoxy)-10-methyl-8H, 10H-7-oxa-1,3,6,10-tetraazaspiro[cyclohepta[de]naphthalene-9,3′-oxetan]-1(10a), 2,3a, 3a¹(6a), 5-pentaene

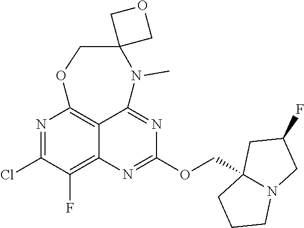 [SMILES: CN1c2nc(OC[C@@]34CCCN3C[C@H](F)C4)nc3c(F)c(Cl)nc(c23)OCC12COC2]

To a solution of 5-chloro-4-fluoro-10-methyl-2-(methylthio)-8H, 10H-7-oxa-1,3,6,10-tetraazaspiro[cyclohepta[de]naphthalene-9,3′-oxetan]-1(10a), 2,3a, 3a¹(6a), 5-pentaene (200 mg, 0.58 mmol) in DCM (15 mL) was added m-CPBA (99.7 mg, 0.58 mmol) at rt., the mixture was stirred at rt for 1 h as mixture 1. Meanwhile, to a solution of ((2R,7aS)-2-fluorotetrahydro-1H-pyrrolizin-7a(5H)-yl) methanol (277 mg, 1.74 mmol) in 15 mL THF was added LiHMDS (1 N in THF, 1,16 mL, 1,16 mmol) at room temperature and it was stirred at room temperature for 1 hour as the mixture 2. Then, the mixture 2 was added to mixture 1 at room temperature and the mixture was stirred at room temperature for 2 hours After completion, the reaction mixture was evaporated. The residue was purified by chromatography column on silica (DCM: MeOH=10:1) to give the title product (145 mg). MS (ESI, m e) [M+H]⁺ 454.2.

Step 4: 3-chloro-5-(4-fluoro-2-(((2R,7aS)-2-fluorotetrahydro-1H-pyrrolizin-7a(5H)-yl) methoxy)-10-methyl-8H, 10H-7-oxa-1,3,6,10-tetraazaspiro[cyclohepta[de]naphthalene-9,3′-oxetan]-1(10a), 2,3a, 3a¹(6a), 5-pentaen-5-yl)-4-(trifluoromethyl)aniline

To a solution of 5-chloro-4-fluoro-2-(((2R,7aS)-2-fluorotetrahydro-1H-pyrrolizin-7a(5H)-yl) methoxy)-10-methyl-8H, 10H-7-oxa-1,3,6,10-tetraazaspiro[cyclohepta[de]naphthalene-9,3′-oxetan]-1(10a), 2,3a, 3a¹(6a), 5-pentaene (55 mg, 0.12 mmol) in dioxane/H₂O (10/2 mL) was added 3-chloro-5-(4,4,5,5-tetramethyl-1,3,2-dioxaborolan-2-yl)-4-(trifluoromethyl) aniline (77 mg, 0.24 mmol), NaHCO₃(30 mg, 0.35 mmol) and Pd(dtbpf)Cl₂ (7.8 mg, 0.012 mmol) at room temperature, the mixture was stirred at 95° C. for 3 hours. The resulting cooled mixture was concentrated and purified by column chromatography (DCM/MeOH=10/1) to give a residue which was further purified by Prep-HPLC to give the title product (5.29 mg). ¹H NMR (500 MHZ, CD₃OD) δ6.92-6.84 (m, 1H), 6.52-6.42 (m, 1H), 5.44-5.26 (m, 1H), 5.13-5.11 (m, 2H), 4.93-4.90 (m, 2H), 4.74-4.69 (m, 2H), 4.42-4.33 (m, 2H), 3.66 (s, 3H), 3.45-3.35 (m, 3H), 3.13-3.08 (m, 1H), 2.44-1.89 (m, 6H). MS (ESI, m/e) [M+H]⁺ 613.2.

Example 24: 3-chloro-5-(4′-fluoro-2′-(((2R,7aS)-2-fluorotetrahydro-1H-pyrrolizin-7a(5H)-yl) methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaen-5′-yl)-4-(trifluoromethyl)aniline

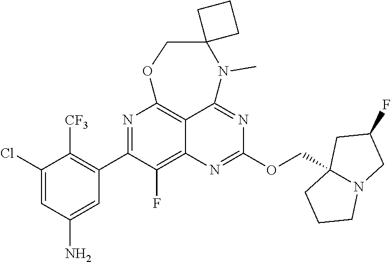 [SMILES: CN1c2nc(OC[C@@]34CCCN3C[C@H](F)C4)nc3c(F)c(-c4cc(N)cc(Cl)c4C(F)(F)F)nc(c23)OCC12CCC2]

Step 1: (1-(methylamino) cyclobutyl) methanol

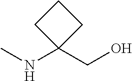

To a solution of 1-((tert-butoxycarbonyl) amino) cyclobutane-1-carboxylic acid (1.5 g. 6.98 mmol) in THF (25 mL) was added LiAIH₄ (663 mg, 17.48 mmol) at 0° C., the mixture was stirred at 70° C. overnight. Upon completion, the mixture was cooled to room temperature, sodium sulfate decahydrate was added and stirred for 0.5 h. The resulting mixture was filted, and the organic phase was concentrated to give a residue as the title compound (721 mg crude). MS (ESI, m/e) [M+H]⁺ 130.1.

Step 2: (1-((5,7-dichloro-8-fluoro-2-(methylthio) pyrido[4,3-d]pyrimidin-4-yl) (methyl) amino) cyclobutyl) methanol

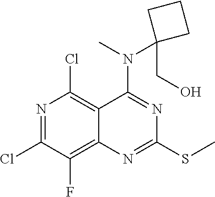 [SMILES: CSc1nc(N(C)C2(CO)CCC2)c2c(Cl)nc(Cl)c(F)c2n1]

To a solution of 4,5,7-trichloro-8-fluoro-2-(methylthio) pyrido[4,3-d]pyrimidine (1540 mg, 2.6 mmol) in DCM (25 mL) was added DIPEA (671 mg, 5.2 mmol) and (1-(methylamino) cyclobutyl) methanol (300 mg, 2.6 mmol) at 0° C. The resulting mixture was stirred at 0° C. to room temperature for 1 hour. After completion, the reaction mixture was concentrated to give the residue. The residue was purified by flash column silica (PE: EtOAc=1:1) to give the title product (498 mg). MS (ESI, m/e) [M+H]⁺ 377.1.

Step 3: 5′-chloro-4′-fluoro-10′-methyl-2′-(methylthio)-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene

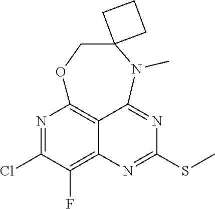 [SMILES: CSc1nc2c3c(nc(Cl)c(F)c3n1)OCC1(CCC1)N2C]

To a solution of (1-((5,7-dichloro-8-fluoro-2-(methylthio) pyrido[4,3-d]pyrimidin-4-yl) (methyl) amino) cyclobutyl) methanol (498 mg, 1.32 mmol) in THF (25 mL) was added LiHMDS (1.7 mL, 1.7 mmol) at room temperature, the mixture was stirred at r.t. for 2 hrs. Upon completion, the mixture was evaporated. The residue was purified by chromatography column on silica (PE: EtOAc=1:2) to give the title product (97 mg). MS (ESI, m/e) [M+H]⁺ 341,1.

Step 4: 5′-chloro-4′-fluoro-2′-(((2R,7aS)-2-fluorotetrahydro-1H-pyrrolizin-7a(5H)-yl) methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene

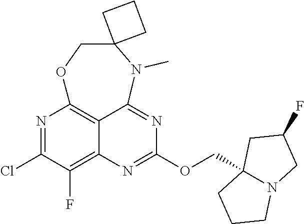 [SMILES: CN1c2nc(OC[C@@]34CCCN3C[C@H](F)C4)nc3c(F)c(Cl)nc(c23)OCC12CCC2]

To a solution of 5′-chloro-4′-fluoro-10′-methyl-2′-(methylthio)-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene (97 mg, 0.285 mmol) in DCM (10 mL) was added m-cpba (49 mg, 0.285 mmol) at rt, the mixture was stirred at rt. for 1 h as mixture 1. Meanwhile, to a solution of ((2R,7aS)-2-fluorotetrahydro-1H-pyrrolizin-7a(5H)-yl) methanol (136 mg, 0.855 mmol) in 10 mL THF was added LiHMDS (1 N in THF, 0.6 mL, 0.6 mmol) at room temperature and it was stirred at room temperature for 1 hour as the mixture 2. Then the mixture 2 was added to mixture 1 at room temperature and the mixture was stirred at room temperature for 2 hours. After completion, the reaction mixture was evaporated. The residue was purified by chromatography column on silica (DCM: MeOH=10:1) to give the title product (75 mg). MS (ESI, m/e) [M+H]⁺ 452,2.

Step 5: 3-chloro-5-(4′-fluoro-2′-(((2R,7aS)-2-fluorotetrahydro-1H-pyrrolizin-7a(5H)-yl) methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaen-5′-yl)-4-(trifluoromethyl)aniline

To a solution of 5′-chloro-4′-fluoro-2′-(((2R,7aS)-2-fluorotetrahydro-1H-pyrrolizin-7a(5H)-yl) methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene (45 mg, 0.1 mmol) in dioxane/H₂O (10/2 mL) was added 3-chloro-5-(4,4,5,5-tetramethyl-1,3,2-dioxaborolan-2-yl)-4-(trifluoromethyl) aniline (80 mg, 0.25 mmol), NaHCO₃ (25 mg, 0.3 mmol) and Pd(dtbpf)Cl₂ (13 mg, 0.02 mmol) at room temperature, the mixture was stirred at 95° C. for 3 hours. The resulting cooled mixture was concentrated and purified by column chromatography (DCM MeOH=10/1) to give a residue which was further purified by Prep-HPLC to give the title product (9 mg). ¹H NMR (500 MHZ, CD₃OD) δ6.92-6.84 (m, 1H), 6.49 (s, 1H), 5.49-5.30 (m, 1H), 4.71-4.59 (m, 2H), 4.44-4.36 (m, 2H), 3.59-3.39 (m, 6H), 3.20-3.15 (m, 1H), 2.76-2.65 (m, 2H), 2.50-1.93 (m, 10H). MS (ESI, m/e) [M+H]⁺ 611.2.

Example 25: 5-ethyl-6-fluoro-4-(4-fluoro-2-(((2R,7aS)-2-fluorotetrahydro-1H-pyrrolizin-7a(5H)-yl) methoxy)-10,11-dimethyl-8,9,10,11-tetrahydro-7-oxa-1,3,6,11-tetraazacycloocta[de]naphthalen-5-yl) naphthalen-2-ol

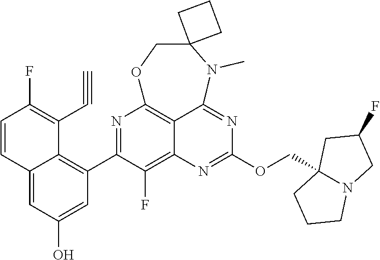 [SMILES: C#Cc1c(F)ccc2cc(O)cc(-c3nc4c5c(nc(OC[C@@]67CCCN6C[C@H](F)C7)nc5c3F)N(C)C3(CCC3)CO4)c12]

Step 1: 4′-fluoro-5′-(7-fluoro-3-(methoxymethoxy)-8-((triisopropylsilyl)ethynyl)naphthalen-1-yl)-2′-(((2R,7aS)-2-fluorotetrahydro-1H-pyrrolizin-7a(5H)-yl)methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene

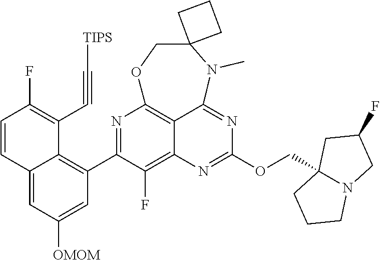

To a solution of 5′-chloro-4′-fluoro-2′-(((2R,7aS)-2-fluorotetrahydro-1H-pyrrolizin-7a(5H)-yl)methoxy)-10′-methyl-8′H.10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene (30 mg, 0.067 mmol) in dioxane/H₂O (10/2 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (86 mg, 0.17 mmol), NaHCO₃(17 mg, 0.2 mmol) and Pd(dtbpf)Cl₂ (5 mg, 0.007 mmol) at room temperature, the mixture was stirred at 95° C. for 3 hours. The resulting cooled mixture was concentrated and purified by column chromatography (DCM MeOH=10/1) to give the title product (25 mg). MS (ESI, m/e) [M+H]⁺ 802.4.

Step 2: 5′-(8-ethynyl-7-fluoro-3-(methoxymethoxy) naphthalen-1-yl)-4′-fluoro-2′-(((2R,7aS)-2-fluorotetrahydro-1H-pyrrolizin-7a(5H)-yl) methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene

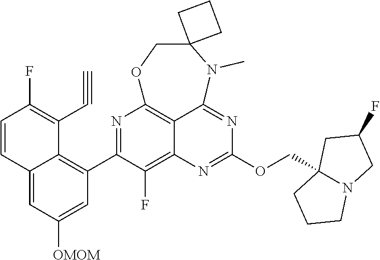

To a solution of 4′-fluoro-5′-(7-fluoro-3-(methoxymethoxy)-8-((triisopropylsilyl)ethynyl)naphthalen-1-yl)-2′-(((2R,7aS)-2-fluorotetrahydro-1H- pyrrolizin-7a(5H)-yl)methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene (25 mg, 0.03 mmol) in DMF (4 mL) was added CsF (23 mg, 0.15 mmol) at room temperature. Upon completion, the mixture was diluted with water (10 mL). Aqueous layer was extracted with DCM (20 mL*3), and the combined organic layer was concentrated to give a residue as the title compound (30 mg crude). MS (ESI, m/e) [M+H]⁺ 646.3.

Step 3: 5-ethyl-6-fluoro-4-(4-fluoro-2-(((2R,7aS)-2-fluorotetrahydro-1H-pyrrolizin-7a(5H)-yl) methoxy)-10,11-dimethyl-8,9,10,11-tetrahydro-7-oxa-1,3,6,11-tetraazacycloocta[de]naphthalen-5-yl) naphthalen-2-ol

To a solution of 5′-(8-ethynyl-7-fluoro-3-(methoxymethoxy) naphthalen-1-yl)-4′-fluoro-2′-(((2R,7aS)-2-fluorotetrahydro-1H-pyrrolizin-7a(5H)-yl) methoxy)-10′-methyl-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaene (30 mg, 0.046 mmol) in DCM(2 mL) was added HCl (4N in dioxane, 1 mL) at room temperature, the mixture was stirred at r.t. for 2 hrs. The resulting mixture was concentrated at room temperature and pH was adjusted to 7 with Na₂CO₃, and the organic layer was concentrated to give a residue which was further purified by Prep-HPLC to give title product (7.9 mg). ¹H NMR (500 MHZ, CD₃OD) δ7.85-7.82 (m, 1H), 7.35-7.18 (m, 3H), 5.49-5.30 (m, 1H), 4.72-4.64 (m, 2H), 4.48-4.37 (m, 2H), 3.59-3.40 (m, 7H), 3.20-3.15 (m, 1H), 2.85-2.78 (m, 1H), 2.72-2.62 (m, 1H), 2.49-1.96 (m, 10H). MS (ESI, m/e) [M+H]⁺ 602.3.

Example 26: 3-chloro-5-((R)-4-fluoro-2-(((2R,7aS)-2-fluorotetrahydro-1H-pyrrolizin-7a(5H)-yl)methoxy)-10,11-dimethyl-8,9,10,11-tetrahydro-7-oxa-1,3,6,11-tetraazacycloocta[de]naphthalen-5-yl)-4-(trifluoromethyl)aniline

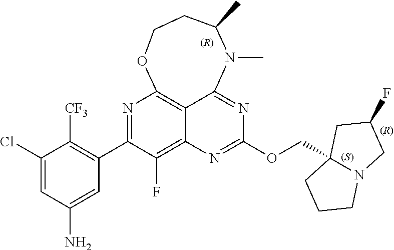

Example 26 was prepared by a procedure similar to that described in Example 6 by replacing 3-(methylamino) butan-1-ol with (R)-3-(methylamino) butan-1-ol to give the title product (6.4 mg). ¹H NMR (500 MHz, CD₃OD) δ6.89-6.87 (m, 1H), 6.68-6.30 (m, 1H), 5.45-5.27 (m, 1H), 4.53-4.22 (m, 4H), 4.10-3.98 (m, 1H), 3.50-3.33 (m, 3H), 3.29 (s, 3H), 3.15-3.07 (m, 1H), 2.46-1.90 (m. 8H), 1.49-1.39 (m, 3H). MS (ESI, m e) [M+H]⁺ 599.2.

Example 27: 3-chloro-5-(4-fluoro-2-(((2R,7aS)-2-fluorotetrahydro-1H-pyrrolizin-7a(5H)-yl)methoxy)-12-methyl-8a, 9,10,11,11a, 12-hexahydro-8H-7-oxa-1,3,6,10,12-pentaazacyclopenta[5,6]cycloocta[1,2,3-de]naphthalen-5-yl)-4-(trifluoromethyl)aniline

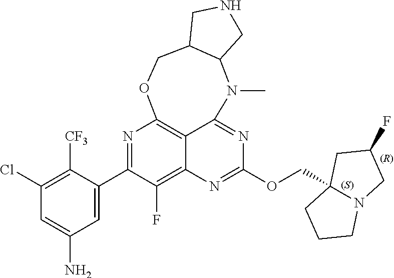

Step 1: tert-butyl 3-(((7-chloro-8-fluoro-4-hydroxy-2-(methylthio)pyrido[4,3-d]pyrimidin-5-yl)oxy)methyl)-4-(methylamino)pyrrolidine-1-carboxylate

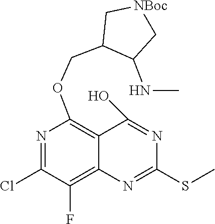 [SMILES: CNC1CN(C(=O)OC(C)(C)C)CC1COc1nc(Cl)c(F)c2nc(SC)nc(O)c12]

To a mixture of tert-butyl 3-(hydroxymethyl)-4-(methylamino)pyrrolidine-1-carboxylate (253 mg, 1,1 mmol) in THF (50 mL) was added NaH (48 mg, 2 mmol), 5,7-dichloro-8-fluoro-2-(methylthio)pyrido[4,3-d]pyrimidin-4-ol (279 mg, 1 mmol). The mixture was stirred for 3 hours at 40° C. Then, the cooled resulting mixture was concentrated. The residue was purified by column chromatography (DCM MeOH=10/1) to give the product (420 mg). MS (ESI, m/e) [M+H]⁺ 474.4.

Step 2: tert-butyl 5-chloro-4-fluoro-12-methyl-2-(methylthio)-8a, 9,11a, 12-tetrahydro-8H-7-oxa-1,3,6,10,12-pentaazacyclopenta[5,6]cycloocta[1,2,3-de]naphthalene-10(11H)-carboxylate

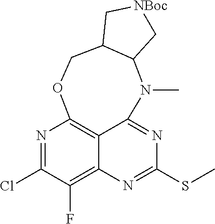 [SMILES: CSc1nc2c3c(nc(Cl)c(F)c3n1)OCC1CN(C(=O)OC(C)(C)C)CC1N2C]

To a solution of tert-butyl 3-(((7-chloro-8-fluoro-4-hydroxy-2-(methylthio)pyrido[4,3-d]pyrimidin-5-yl)oxy)methyl)-4-(methylamino)pyrrolidine-1-carboxylate (420 mg, 0.888 mmol) in DCM (40 mL) was added HATU (675 mg, 1.776 mmol) and DIPEA (0.76 mL, 4.44 mmol), and stirred at room temperature for 12 hours. The resulting cooled solution was concentrated and purified by pre-TLC (Petroleum ether: EtOAc=1:1) to give the title product (80 mg). MS (ESI, m/e) [M+H]⁺ 456.4.

Step 3: tert-butyl 5-chloro-4-fluoro-12-methyl-2-(methylsulfinyl)-8a, 9,11a, 12-tetrahydro-8H-7-oxa-1,3,6,10,12-pentaazacyclopenta[5,6]cycloocta[1,2,3-de]naphthalene-10(11H)-carboxylate

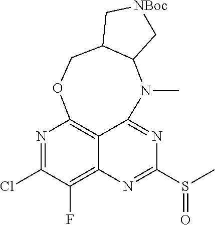 [SMILES: CN1c2nc(S(C)=O)nc3c(F)c(Cl)nc(c23)OCC2CN(C(=O)OC(C)(C)C)CC21]

The mixture of tert-butyl 5-chloro-4-fluoro-12-methyl-2-(methylthio)-8a, 9,11a, 12-tetrahydro-8H-7-oxa-1,3,6,10,12-pentaazacyclopenta[5,6]cycloocta[1,2,3-de]naphthalene-10(11H)-carboxylate (91 mg, 0.2 mmol) and m-CPBA (42 mg, 0.24 mmol) in DCM (4 mL) was stirred at 0° C. for 60 mins. The resulting cooled mixture was used directly next step without more purification. MS (ESI, m/e) [M+H]⁺ 472.5.

Step 4: tert-butyl 5-chloro-4-fluoro-2-(((2R,7aS)-2-fluorotetrahydro-1H-pyrrolizin-7a(5H)-yl)methoxy)-12-methyl-8a, 9,11a, 12-tetrahydro-8H-7-oxa-1,3,6,10,12-pentaazacyclopenta[5,6] cycloocta[1,2,3-de]naphthalene-10(11H)-carboxylate

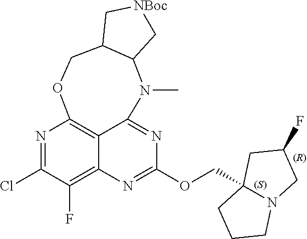

To a solution of ((2R,7aS)-2-fluorotetrahydro-1H-pyrrolizin-7a(5H)-yl)methanol (64 mg, 0.4 mmol) in THF (2 mL) was added LiHMDS (1M, 0.4 mL, 0.4 mmol) at 0° C. for 10 mins. Then the resulting solution of step 3 was decanted into this reaction mixture, stirred for 30 mins, and concentrated. The residue was purified by pre-TLC (DCM MeOH=10/1) to give the title product (80 mg). MS (ESI, m/e) [M+H]⁺ 567.4.

Step 5: tert-butyl 5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-12-methyl-8a, 9,11a, 12-tetrahydro-8H-7-oxa-1,3,6,10,12-pentaazacyclopenta[5.6]cycloocta[1,2,3-de]naphthalene-10(11H)-carboxylate

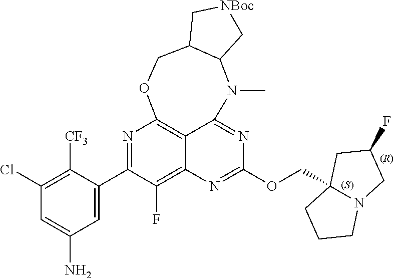

To a mixture of tert-butyl 5-chloro-4-fluoro-2-(((2R,7aS)-2-fluorotetrahydro-1H-pyrrolizin-7a(5H)-yl)methoxy)-12-methyl-8a, 9,11a, 12-tetrahydro-8H-7-oxa-1,3,6,10,12-pentaazacyclopenta[5,6] cycloocta[1,2,3-de]naphthalene-10(11H)-carboxylate (80 mg, 0.142 mmol), NaHCO₃(34 mg, 0.426 mmol), Pd(dtbpf)Cl₂ (9.2 mg, 0.0142 mmol) and 3-chloro-5-(4,4,5,5-tetramethyl-1,3,2-dioxaborolan-2-yl)-4-(trifluoromethyl)aniline (136 mg, 0.426 mmol) was added 1,4-dioxane (5 mL) and water (1 mL), and the mixture was stirred at 90° C. for 2 hours. The resulting cooled mixture was concentrated and purified by pre-TLC (DCM/MeOH=10/1) to give the title product (79 mg). MS (ESI, m/e) [M+H]⁺ 726.6.

Step 6: 3-chloro-5-(4-fluoro-2-(((2R,7aS)-2-fluorotetrahydro-1H-pyrrolizin-7a(5H)-yl)methoxy)-12-methyl-8a, 9,10,11,11a, 12-hexahydro-8H-7-oxa-1,3,6,10,12-pentaazacyclopenta[5,6]cycloocta[1,2,3-de]naphthalen-5-yl)-4-(trifluoromethyl)aniline

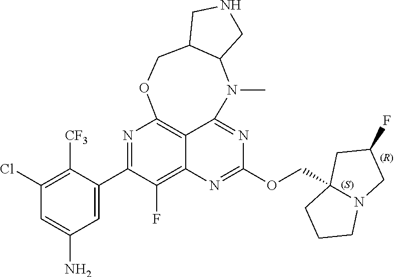

To a solution tert-butyl 5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-12-methyl-8a, 9,11a, 12-tetrahydro-8H-7-oxa-1,3,6,10,12-pentaazacyclopenta[5,6]cycloocta[1,2,3-de]naphthalene-10(11H)-carboxylate (22 mg, 0.03 mmol) in 4 mL dichloromethane was added 4 mL trifluoroacetic acid. The mixture was stirred at room temperature for 2 hrs. Then it was evaporated below 35° C. The residue was purified by prep-HPLC to give the title product (3.0 mg). ¹H NMR (500 MHz, CD₃OD) δ6.93-6.88 (m, 1H), 6.58-6.40 (m, 1H), 5.65-5.45 (m, 1H), 4.75-4.51 (m, 5H), 4.08-3.49 (m, 10H), 3.10-3.03 (m, 1H), 2.61-2.11 (m, 6H). MS (ESI, m/e) [M+H]⁺ 626.5.

Example 28: 2-amino-4-(9′-chloro-11′-fluoro-2′-(((2R,7aS)-2-fluorotetrahydro-1H-pyrrolizin-7a(5H)-yl)methoxy)-4′-methyl-6′,7′-dihydro-4′H-spiro[oxetane-3,5′-[1,5]oxazocino[4,3,2-de]quinazolin]-10′-yl)-7-fluorobenzo[b]thiophene-3-carbonitrile

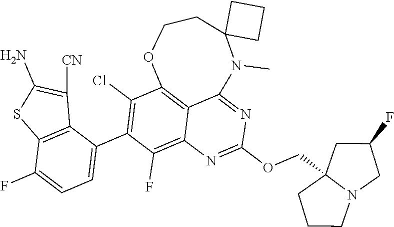 [SMILES: CN1c2nc(OC[C@@]34CCCN3C[C@H](F)C4)nc3c(F)c(-c4ccc(F)c5sc(N)c(C#N)c45)c(Cl)c(c23)OCCC12CCC2]

Example 28 was prepared by a procedure similar to that described in Example 19 by replacing (R)-3-(methylamino)butan-1-ol with 2-(3-(methylamino)oxetan-3-yl)ethan-1-ol to give the title product (25 mg). ¹H NMR (500 MHZ, DMSO-d₆) δ 8.10 (s, 2H), 7.29-7.05 (m, 2H), 5.60-5.40 (m, 1H), 4.80-4.13 (m, 8H), 3.18 (s, 3H), 2.64-2.53 (m, 3H), 2.47-1.90 (m, 6H). [M+H]⁺ 657.5.

Example 29: 3-chloro-5-(4-fluoro-2-(((2R,7aS)-2-fluorotetrahydro-1H-pyrrolizin-7a(5H)-yl)methoxy)-10-methyl-8H, 10H-7-oxa-1,3,6,10-tetraazaspiro[cyclohepta[de]naphthalene-9,1′-cyclopropan]-1(10a), 2,3a, 3 al(6a), 5-pentaen-5-yl)-4-(trifluoromethyl)aniline

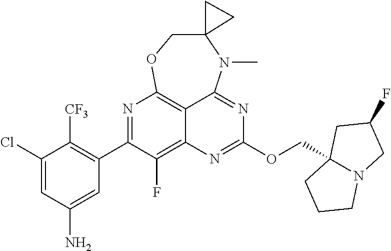 [SMILES: CN1c2nc(OC[C@@]34CCCN3C[C@H](F)C4)nc3c(F)c(-c4cc(N)cc(Cl)c4C(F)(F)F)nc(c23)OCC12CC2]

Example 29 was prepared by a procedure similar to that described in Example 23 by replacing (3-(methylamino) oxetan-3-yl) methanol with (1-(methylamino)cyclopropyl)methanol to give the title product (12 mg). ¹H NMR (500 MHZ, CD₃OD) δ6.92-6.84 (m, 1H), 6.56-6.45 (m, 1H), 5.45-5.24 (m, 1H), 4.57-4.52 (m, 2H), 4.41-4.27 (m, 2H), 3.47-3.32 (m, 6H), 3.13-3.08 (m, 1H), 2.42-1.92 (m, 6H), 1.29-1.20 (m, 4H). MS (ESI, m/e) [M+H]⁺ 597.2.

Example 30: 3-chloro-5-(4-fluoro-2-(((2R,7aS)-2-fluorotetrahydro-1H-pyrrolizin-7a(5H)-yl)methoxy)-11-methyl-8,9-dihydro-11H-7-oxa-1,3,6,11-tetraazaspiro[cycloocta[de]naphthalene-10,3′-oxetan]-1(11a), 2,3a, 3a¹(6a), 5-pentaen-5-yl)-4-(trifluoromethyl)aniline

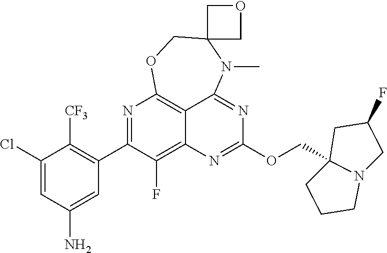 [SMILES: CN1c2nc(OC[C@@]34CCCN3C[C@H](F)C4)nc3c(F)c(-c4cc(N)cc(Cl)c4C(F)(F)F)nc(c23)OCC12COC2]

Step 1: 7-chloro-8-fluoro-5-(2-(3-(methylamino)oxetan-3-yl)ethoxy)-2-(methylthio)pyrido[4,3-d]pyrimidin-4-ol

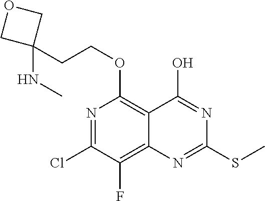 [SMILES: CNC1(CCOc2nc(Cl)c(F)c3nc(SC)nc(O)c23)COC1]

To a stirred solution of 5,7-dichloro-8-fluoro-2-(methylthio)pyrido[4,3-d]pyrimidin-4-ol (560 mg, 1.0 mmol) and 2-(3-(methylamino)oxetan-3-yl)ethan-1-ol (260 mg, 1.0 mmol) in THF (10 mL) was added NaH (560 mg, 7.0 mmol, 60%) at 0° C., and the resulting mixture was stirred for 1 h at room temperature. The reaction mixture was quenched with H₂O, then pH was adjusted to 6 with 1N aq HCl. The resulting mixture was extracted with EtOAc and the combined organic layer was washed with brine, dried over anhydrous Na₂SO₄, and concentrated in vacuo. The residue was purified by flash chromatography (CH₂Cl, MeOH=100:1 to 5:1) to afford the product (660 mg). MS (ESI, m/e) [M+H]⁺ 375.3.

Step 2: 5-chloro-4-fluoro-11-methyl-2-(methylthio)-8,9-dihydro-11H-7-oxa-1,3,6,11-tetraazaspiro[cycloocta[de]naphthalene-10,3′-oxetan]-1(11a),2,3a, 3a¹(6a), 5-pentaene

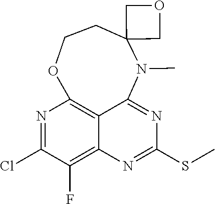 [SMILES: CSc1nc2c3c(nc(Cl)c(F)c3n1)OCCC1(COC1)N2C]

To a stirred solution of 7-chloro-8-fluoro-5-(2-(3-(methylamino)oxetan-3-yl)ethoxy)-2-(methylthio)pyrido[4,3-d]pyrimidin-4-ol (375 mg, 1.0 mmol) in ACN (50 mL) was added DIPEA (323 mg, 2.5 mmol) and bis(2-oxo-3-oxazolidinyl)phosphinic chloride (BopC1) (760 mg, 3 mmol) at room temperature, and the resulting mixture was stirred for 2 hrs at 70° C. The reaction mixture was concentrated and purified by flash chromatography (CH₂Cl₂/EtOAc=4:1 to 1:3) to afford the desired product (240 mg). MS (ESI, m/e) [M+H]⁺ 357.3.

Step 3: 5-chloro-4-fluoro-11-methyl-2-(methylsulfinyl)-8,9-dihydro-11H-7-oxa-1,3,6,11-tetraazaspiro[cycloocta[de]naphthalene-10,3′-oxetan]-1(11a), 2,3a, 3a¹(6a), 5-pentaene

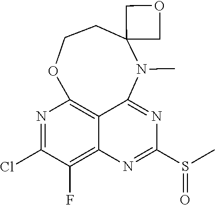 [SMILES: CN1c2nc(S(C)=O)nc3c(F)c(Cl)nc(c23)OCCC12COC2]

To a stirred solution of 5-chloro-4-fluoro-11-methyl-2-(methylthio)-8,9-dihydro-11H-7-oxa-1,3,6,11-tetraazaspiro[cycloocta[de]naphthalene-10,3′-oxetan]-1(11a), 2,3a, 3a¹(6a), 5-pentaene (260 mg, 0.72 mmol) in CH₂Cl₂ (2.5 mL) was added m-CPBA (200 mg, 1 mmol, 85%) at 0° C., and the resulting mixture was stirred at 0° C. for 10 min. The reaction mixture was used in the next step without further purification.

Step 4: 5-chloro-4-fluoro-2-(((2R,7aS)-2-fluorotetrahydro-1H-pyrrolizin-7a(5H)-yl)methoxy)-11-methyl-8,9-dihydro-11H-7-oxa-1,3,6,11-tetraazaspiro[cycloocta[de]naphthalene-10,3′-oxetan]-1(11a), 2,3a, 3a¹(6a), 5-pentaene

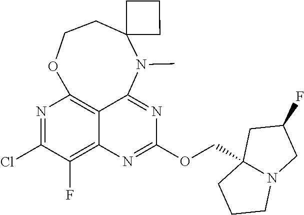 [SMILES: CN1c2nc(OC[C@@]34CCCN3C[C@H](F)C4)nc3c(F)c(Cl)nc(c23)OCCC12CCC2]

To a stirred solution of 5-chloro-4-fluoro-11-methyl-2-(methylsulfinyl)-8,9-dihydro-11H-7-oxa-1,3,6,11-tetraazaspiro[cycloocta[de]naphthalene-10,3′-oxetan]-1(11a), 2,3a, 3a′(6a), 5-pentaene (0.72 mmol) was added ((2R,7aS)-2-fluorotetrahydro-1H-pyrrolizin-7a(5H)-yl)methanol (240 mg, 1.5 mmol) in THF (2.5 mL) and LiHMDS (2 mL, 2 mmol, 1M in THF) dropwise at 0° C., and the resulting mixture was stirred at 0° C. for 20 min. The reaction was quenched by H₂O and concentrated in vacuo. The residue was purified by flash chromatography (CH₂Cl₂/MeOH=100:1 to 30:1) to afford the product (190 mg). MS (ESI, m/e) [M+H]⁺ 468.5.

Step 5: 3-chloro-5-(4-fluoro-2-(((2R,7aS)-2-fluorotetrahydro-1H-pyrrolizin-7a(5H)-yl)methoxy)-11-methyl-8,9-dihydro-11H-7-oxa-1,3,6,11- tetraazaspiro[cycloocta[de]naphthalene-10,3′-oxetan]-1(11a), 2,3a, 3a¹(6a), 5-pentaen-5-yl)-4-(trifluoromethyl)aniline

To a mixture of 5-chloro-4-fluoro-2-(((2R,7aS)-2-fluorotetrahydro-1H-pyrrolizin-7a(5H)-yl)methoxy)-11-methyl-8,9-dihydro-11H-7-oxa-1,3,6,11-tetraazaspiro[cycloocta[de]naphthalene-10,3′-oxetan]-1(11a), 2,3a, 3a¹(6a), 5-pentaene (100 mg), 3-chloro-5-(4,4,5,5-tetramethyl-1,3,2-dioxaborolan-2-yl)-4-(trifluoromethyl)aniline (160 mg, 0.5 mmol), Pd(dtbpf)Cl₂ (35 mg, 0.05 mmol) and NaHCO₃(84 mg, 1 mmol) was added dioxane (10.0 mL) and H₂O (2 mL), and the reaction mixture was stirred at 90° C. for 2 hrs. The reaction mixture was concentrated and purified by flash chromatography (DCM/MeOH=100:1 to 10:1), then further purified by Prep-HPLC to give the title product (6.3 mg). ¹H NMR (500 MHZ, CD₃OD) δ6.91-6.83 (m, 1H), 6.65-6.37 (m, 1H), 5.53-5.30 (m, 1H), 4.72-4.35 (m, 7H), 3.67-3.42 (m, 3H), 3.26 (s, 3H), 3.25-3.18 (m, 1H), 2.55-1.93 (m, 8H). [M+H]⁺ 627.3.

Example 31: 3-chloro-5-(4-fluoro-2-(((2R,7aS)-2-fluorotetrahydro-1H-pyrrolizin-7a(5H)-yl) methoxy)-11-methyl-8,9-dihydro-11H-7-oxa-1,3,6,11-tetraazaspiro[cycloocta[de]naphthalene-10,1′-cyclopropan]-1(11a), 2,3a, 4,6-pentaen-5-yl)-4 (trifluoromethyl)aniline

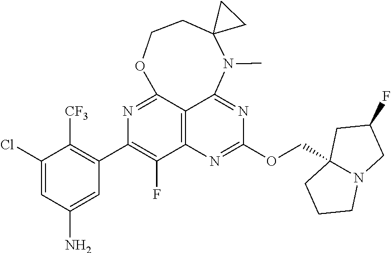 [SMILES: CN1c2nc(OC[C@@]34CCCN3C[C@H](F)C4)nc3c(F)c(-c4cc(N)cc(Cl)c4C(F)(F)F)nc(c23)OCCC12CC2]

Example 31 was prepared by a procedure similar to that described in Example 30 by replacing 2-(3-(methylamino)oxetan-3-yl)ethan-1-ol with 2-(1-(methylamino) cyclopropyl) ethan-1-ol to give the title product (7.8 mg). ¹H NMR (500 MHZ, CD₃OD) δ 6.92-6.79 (m, 1H), 6.69-6.45 (m, 1H), 5.42-5.24 (m, 1H), 4.62-4.41 (m, 2H), 4.38-4.16 (m, 2H), 3.38-3.35 (m, 3H), 3.09-3.02 (m, 1H), 2.66-1.54 (m, 8H), 1.37-1.27 (m, 3H), 1.02-0.83 (m, 4H). MS (ESI, m/e) [M+H]⁺ 611.2.

Example 32: 3-chloro-5-(4′-fluoro-2′-(((2R,7aS)-2-fluorotetrahydro-1H-pyrrolizin-7a(5H)-yl)methoxy)-11′-methyl-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaen-5′-yl)-4-(trifluoromethyl)aniline

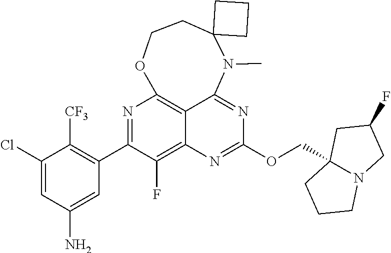 [SMILES: CN1c2nc(OC[C@@]34CCCN3C[C@H](F)C4)nc3c(F)c(-c4cc(N)cc(Cl)c4C(F)(F)F)nc(c23)OCCC12CCC2]

Example 32 was prepared by a procedure similar to that described in Example 30 by replacing 2-(3-(methylamino)oxetan-3-yl)ethan-1-ol with 2-(1-(methylamino) cyclobutyl) ethan-1-ol to give the title product (12.6 mg). ¹H NMR (500 MHZ, CD₃OD) δ 6.90-6.84 (m, 1H), 6.65-6.25 (m, 1H), 5.49-5.30 (m, 1H), 4.56-4.33 (m, 4H), 3.59-3.38 (m, 3H), 3.24 (s, 3H), 3.19-3.14 (m, 1H), 2.68-1.61 (m, 14H). MS (ESI, m/e) [M+H]⁺ 625.2.

Example 33: 5′-(5-amino-3-chloro-2-(trifluoromethyl)phenyl)-4′-fluoro-2′-(((2R,7aS)-2-fluorotetrahydro-1H-pyrrolizin-7a(5H)-yl)methoxy)-11′-methyl-8′,9′-dihydro-11′H-7′-oxa-1′,3′,6′,11′-tetraazaspiro[cyclobutane-1,10′-cycloocta[de]naphthalen]-1′(11a′), 2′0.3a′, 3 al′(6a′), 5′-pentaen-3-ol

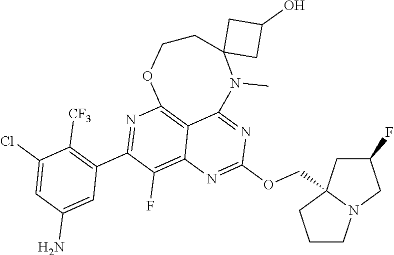 [SMILES: CN1c2nc(OC[C@@]34CCCN3C[C@H](F)C4)nc3c(F)c(-c4cc(N)cc(Cl)c4C(F)(F)F)nc(c23)OCCC12CC(O)C2]

Step 1: ethyl 2-(3-(benzyloxy)cyclobutylidene)acetate

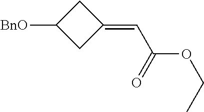 [SMILES: CCOC(=O)C=C1CC(OCc2ccccc2)C1]

A mixture of 3-(benzyloxy)cyclobutan-1-one (5 g, 28.37 mmol) and ethyl 2-(triphenyl-15-phosphaneylidene)acetate (11.86 g, 34.05 mmol) in DCM (100 mL) was stirred at 40° C. for 16 hrs. The reaction mixture was quenched by water (5 mL), and then diluted with water (100 mL) and extracted with EtOAc. The combined organic layers were washed with brine, dried over anhydrous sodium sulfate, filtered and concentrated under reduced pressure. The residue was purified by silica gel chromatography (PE: EtOAc=100:1 to 10:1) to give the title product (4.5 g). MS (ESI, m/e) [M+H]⁺ 247.

Step 2: ethyl 2-(3-(benzyloxy)-1-(methylamino)cyclobutyl) acetate

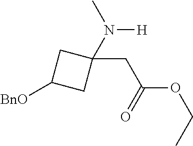 [SMILES: CCOC(=O)CC1(NC)CC(OCc2ccccc2)C1]

Into a 20 mL sealed tube were added ethyl 2-(3-(benzyloxy) cyclobutylidene)acetate (3.5 g, 14.22 mmol), EtOH (3.5 mL) and CH₃NH₂ (30% in EtOH) (1.71 g, 30%, 16.50 mmol). After stirring for 12 hrs at 50° C., the mixture was concentrated. The crude product (3.6 g) was used in the next step directly without further purification. (ESI, m/e) [M+H]⁺ 278.

Step 3: ethyl 2-(3-hydroxy-1-(methylamino)cyclobutyl)acetate

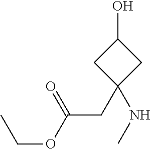

To a solution of ethyl ethyl 2-(3-(benzyloxy)-1-(methylamino) cyclobutyl)acetate (3.6 g, 12.95 mmol) in EtOH (30 mL) was added Pd/C (10%, 3.6 g) at room temperature. After stirring for 6 hrs at 60° C. under H₂(g) atmosphere, the mixture was filtered through a Celite pad and concentrated under reduced pressure. The crude product (2.3 g) was used in the next step directly without further purification. (ESI, m/e) [M+H]⁺ 188.

Step 4: ethyl 2-(3-((tert-butyldimethylsilyl)oxy)-1-(methylamino) cyclobutyl) acetate

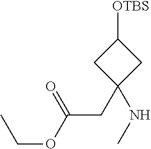 [SMILES: CCOC(=O)CC1(NC)CC(O[Si](C)(C)C(C)(C)C)C1]

To a solution of ethyl 2-(3-hydroxy-1-(methylamino)cyclobutyl) acetate (2.3 g, 12,23 mmol) in DCM (20 mL) were added Imidazole(2.5 g, 36.70 mmol) and TBSCl(2,2 g, 14.68 mmol) at 0° C. After stirring for 3 hrs at room temperature, the mixture was quenched by NH₄Cl(aq) at 0° C., and extracted with EtOAc. The organic layer was dried over anhydrous sodium sulfate and concentrated. The residue was purified by silica gel chromatography (PE:EtOAc=100:0 to 50:50) to give the title product (3.5 g). (ESI, m/e) [M+H]⁺ 302.

Step 5: 2-(3-((tert-butyldimethylsilyl)oxy)-1-(methylamino) cyclobutyl)ethan-1-ol

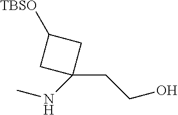 [SMILES: CNC1(CCO)CC(O[Si](C)(C)C(C)(C)C)C1]

To a solution of ethyl 2-(3-((tert-butyldimethylsilyl)oxy)-1-(methylamino)cyclobutyl)acetate (3.2 g, 10.63 mmol) in THF (50 mL) were added LiAlH₄ (0.81 g, 21.26 mmol) at 0° C. After stirring overnight at room temperature, the mixture was quenched by Na₂SO₄·10H₂O at 0° C. The resulting mixture was filtered, the filter cake was washed with DCM/MeOH (3:1). The filtrate was concentrated under reduced pressure. The residue was purified by silica gel chromatography (DCM:MeOH=100:0 to 0:100) to give the title product. MS (ESI, m/e) [M+H]⁺ 260.2.

Step 6: 5-(2-(3-((tert-butyldimethylsilyl)oxy)-1-(methylamino)cyclobutyl)ethoxy)-7-chloro-8-fluoro-2-(methylthio)pyrido[4,3-d]pyrimidin-4-ol

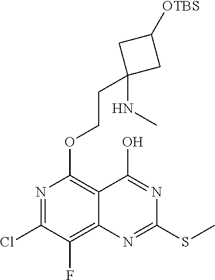 [SMILES: CNC1(CCOc2nc(Cl)c(F)c3nc(SC)nc(O)c23)CC(O[Si](C)(C)C(C)(C)C)C1]

To a stirred solution of 5,7-dichloro-8-fluoro-2-(methylthio)pyrido[4,3-d]pyrimidin-4-ol (280 mg, 1.0 mmol) and 2-(3-((tert-butyldimethylsilyl)oxy)-1-(methylamino)cyclobutyl)ethan-1-ol (260 mg, 1.0 mmol) in THF (10 mL) was added NaH (160 mg, 4.0 mmol, 60%) at 0° C., the resulting mixture was stirred for 1 h at room temperature. The reaction mixture was quenched with H₂O, then adjusted pH to 6 with 1N aq HCl. The mixture was filtered and filter cake was directly used in the next step without further purification. MS (ESI, m/e) [M+H]⁺ 503.4.

Step 7: 3-((tert-butyldimethylsilyl)oxy)-5′-chloro-4′-fluoro-11′-methyl-2′-(methylthio)-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaene

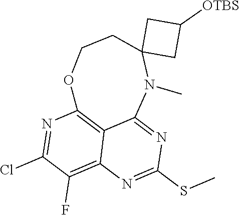 [SMILES: CSc1nc2c3c(nc(Cl)c(F)c3n1)OCCC1(CC(O[Si](C)(C)C(C)(C)C)C1)N2C]

To a stirred solution of 5-(2-(3-((tert-butyldimethylsilyl)oxy)-1-(methylamino)cyclobutyl)ethoxy)-7-chloro-8-fluoro-2-(methylthio)pyrido[4,3-d]pyrimidin-4-ol (1.0 mmol) in MeCN (50 mL) was added DIEA (387 mg, 3.0 mmol) and BOPCl (508 mg, 2.0 mmol) at room temperature, the resulting mixture was stirred for 1 h at 70° C. The reaction mixture was concentrated and purified by flash chromatography (PE/EtOAc=20:1 to 5:1) to afford the desired product (180 mg). MS (ESI, m/e) [M+H]⁺ 485.4.

Step 8: 3-((tert-butyldimethylsilyl)oxy)-5′-chloro-4′-fluoro-11′-methyl-2′-(methylsulfinyl)-8′,9′-dihydro-11′H-7′-oxa-1′,3′,6′, 1 l′-tetraazaspiro[cyclobutane-1,10′-cycloocta[de]naphthalen]-1′(11a′), 2′,3a′, 3 al′(6a′), 5′-pentaene

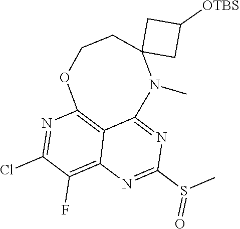 [SMILES: CN1c2nc(S(C)=O)nc3c(F)c(Cl)nc(c23)OCCC12CC(O[Si](C)(C)C(C)(C)C)C2]

To a stirred solution of 3-((tert-butyldimethylsilyl)oxy)-5′-chloro-4′-fluoro-11′-methyl-2′-(methylthio)-8′,9′-dihydro-11′H-7′-oxa-1′,3′,6′,1l′-tetraazaspiro[cyclobutane-1,10′-cycloocta[de]naphthalen]-1′(11a′), 2′,3a′, 3 al′(6a′), 5′-pentaene (180 mg, 0.37 mmol) in DCM (5 mL) was added m-CPBA (76 mg, 0.37 mmol, 85%) at 0° C., the resulting mixture was stirred at 0° C. for 15 min. The reaction was quenched with aq. Na₂S₂O₃, then extracted with DCM. The organic layer was washed with Sat, aq NaHCO3 and brine, dried over anhydrous Na₂SO₄, concentrated in vacuo. The residue was directly used in the next step without further purification.

Step 9: 3-((tert-butyldimethylsilyl)oxy)-5′-chloro-4′-fluoro-2′-(((2R,7aS)-2-fluorotetrahydro-1H-pyrrolizin-7a(5H)-yl)methoxy)-1l′-methyl-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaene

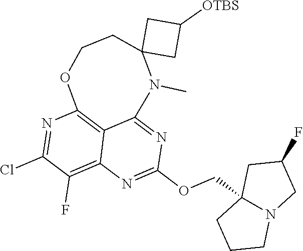 [SMILES: CN1c2nc(OC[C@@]34CCCN3C[C@H](F)C4)nc3c(F)c(Cl)nc(c23)OCCC12CC(O[Si](C)(C)C(C)(C)C)C2]

To a stirred solution of 3-((tert-butyldimethylsilyl)oxy)-5′-chloro-4′-fluoro-11′-methyl-2′-(methylsulfinyl)-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaene (0.37 mmol) and ((2R,7aS)-2-fluorotetrahydro-1H-pyrrolizin-7a(5H)-yl)methanol (118 mg, 0.74 mmol) in THF (8 mL) was added LiHMDS (0.74 mL, 0.74 mmol, 1M in THF) dropwise at 0° C., the resulting mixture was stirred at 0° C. for 20 min. The reaction was quenched with Sat, aq NH₄Cl, then extracted with DCM. The organic layer was washed with brine, dried over anhydrous Na₂SO₄, and concentrated in vacuo. The residue was purified by flash chromatography (DCM/MeOH=100:1 to 30:1) to afford the product (115 mg). MS (ESI, m/e) [M+H]⁺ 596.1.

Step 10: 3-(3-((tert-butyldimethylsilyl)oxy)-4′-fluoro-2′-(((2R,7aS)-2-fluorotetrahydro-1H-pyrrolizin-7a(5H)-yl)methoxy)-11′-methyl-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaen-5′-yl)-5-chloro-4-(trifluoromethyl)aniline

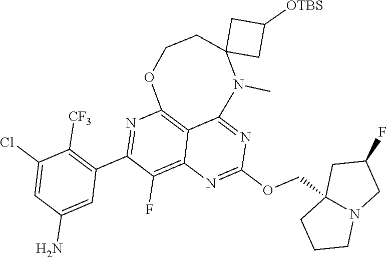 [SMILES: CN1c2nc(OC[C@@]34CCCN3C[C@H](F)C4)nc3c(F)c(-c4cc(N)cc(Cl)c4C(F)(F)F)nc(c23)OCCC12CC(O[Si](C)(C)C(C)(C)C)C2]

To a mixture of 3-((tert-butyldimethylsilyl)oxy)-5′-chloro-4′-fluoro-2′-(((2R,7aS)-2-fluorotetrahydro-1H-pyrrolizin-7a(5H)-yl)methoxy)-1l′-methyl-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaene (115 mg, 0.19 mmol), 3-chloro-5-(4,4,5,5-tetramethyl-1,3,2-dioxaborolan-2-yl)-4-(trifluoromethyl)aniline (73 mg, 0.23 mmol), Pd(dtbpf)Cl₂ (24 mg, 0.04 mmol) and NaHCO₃(32 mg, 0.38 mmol) was added dioxane (5.0 mL) and H₂O (1.0 mL), and the resulting mixture was stirred at 90° C. for 1 h. The reaction mixture was concentrated and purified by flash chromatography (DCM/MeOH=100:1 to 5:1) to give the product (78 mg). MS (ESI, m/e) [M+H]⁺ 755.6.

Step 11: 5′-(5-amino-3-chloro-2-(trifluoromethyl)phenyl)-4′-fluoro-2′-(((2R,7aS)-2-fluorotetrahydro-1H-pyrrolizin-7a(5H)-yl)methoxy)-11′-methyl-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaen-3-ol

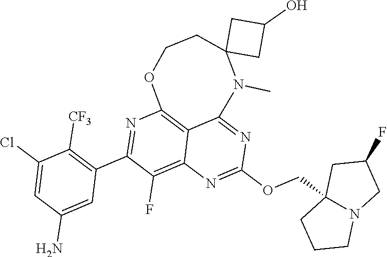 [SMILES: CN1c2nc(OC[C@@]34CCCN3C[C@H](F)C4)nc3c(F)c(-c4cc(N)cc(Cl)c4C(F)(F)F)nc(c23)OCCC12CC(O)C2]

To a mixture of 3-(3-((tert-butyldimethylsilyl)oxy)-4′-fluoro-2′-(((2R,7aS)-2-fluorotetrahydro-1H-pyrrolizin-7a(5H)-yl)methoxy)-1l′-methyl-8′,9′-dihydro-11′H-7′-oxa-1′,3′,6′,11′-tetraazaspiro[cyclobutane-1,10′-cycloocta[de]naphthalen]-1′(11a′), 2′,3a′, 3 al′(6a′), 5′-pentaen-5′-yl)-5-chloro-4-(trifluoromethyl)aniline (78 mg, 0.10 mmol) in DCM (4.0 mL) was added 4N HCl (in dioxane) (4.0 mL). The resulting mixture was stirred for 20 min. The reaction mixture was concentrated and basified with DIEA, then prep-HPLC to give the title product (34.5 mg). ¹H NMR (500 MHZ, CD₃OD) δ6.90-6.81 (m, 1H), 6.73-6.19 (m, 1H), 5.42-5.18 (m, 1H), 4.70-4.38 (m, 2H), 4.36-4.15 (m, 2H), 4.13-3.97 (m, 1H), 3.29 (s, 3H), 3.28-3.12 (m, 3H), 3.09-2.84 (m, 2H), 2.83-2.55 (m, 1H), 2.50-1.80 (m, 10H). MS (ESI, m/e) [M+H]⁺ 641.5.

Example 34: 3-chloro-5-((8S,10R)-4-fluoro-2-(((2R,7aS)-2-fluorotetrahydro-1H-pyrrolizin-7a(5H)-yl)methoxy)-8,10,11-trimethyl-8,9,10,11-tetrahydro-7-oxa-1,3,6,11-tetraazacycloocta[de]naphthalen-5-yl)-4-(trifluoromethyl)aniline

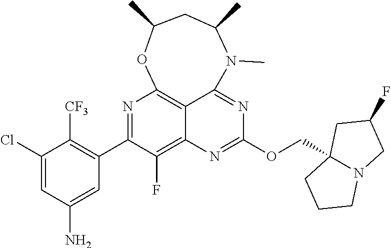 [SMILES: C[C@@H]1C[C@H](C)Oc2nc(-c3cc(N)cc(Cl)c3C(F)(F)F)c(F)c3nc(OC[C@@]45CCCN4C[C@H](F)C5)nc(c23)N1C]

Step 1: (4S,6S)-4,6-dimethyl-1,3,2-dioxathiane 2-oxide

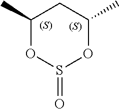

To a solution of (2S,4S)-pentane-2,4-diol (90 g, 0.87 mol) and TEA (262 g, 2.61 mol) in CH₂Cl₂ (4.5 L) at 0° C. was slowly added SOCl₂ (113 g, 0.95 mol). The mixture was allowed to warm up to room temperature and was stirred for an additional 60 minutes. Next, the reaction was quenched by addition of ice water (1.8 L). The two layers were separated, and the aqueous phase was extracted three times with Et₂O (900 mL). The combined organic layers were washed with a 6% NaHCO₃ solution, brine and H₂O respectively. Then the organic layer was dried with Na₂SO₄, filtered and the solvent was removed. The crude product (120 g) was used in the next step directly without further purification.

Step 2: (4S,6S)-4,6-dimethyl-1,3,2-dioxathiane 2,2-dioxide

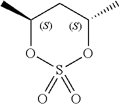

To a cooled solution (0° C.) of a previously prepared (4S,6S)-4,6-dimethyl-1,3,2-dioxathiane 2-oxide (120 g crude) in a mixture of CH₂Cl₂ (900 mL) and MeCN (900 mL) was added RuCl₃ H₂O (2 g) followed by the addition of a solution of NaIO₄ (311 g, 1.44 mol) in water (1.35 L). The resulting two-layer mixture was stirred at 0° C. for 15 minutes and allowed to warm to room temperature and was stirred for an additional 45 minutes during which the reaction mixture turned from dark brown to orange. Next, Et₂O (6 L) was added, and the two phases were separated followed by extraction of the aqueous layer with Et₂O. The organic layers were combined and washed subsequently with a saturated NaHCO₃ solution, brine and water. The organic layer was dried with Na₂SO₄, filtered and the solvent was removed in vacuo. The crude product was re-crystallized from Et₂O/pentanes to give title product (85 g crude).

Step 3: (2S,4R)-4-((4-methoxvbenzyl)(methyl)amino)pentan-2-vl hydrogen sulfate

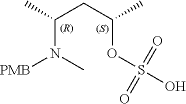

A mixture of [(4-methoxyphenyl) methyl](methyl)amine (155 g, 1.02 mol) and (4S,6S)-4,6-dimethyl-1,3,2-dioxathiane 2,2-dioxide (85 g, 0.51 mol) was stirred for 1 h at 45° C. The crude product (240 g) was used in the next step directly without further purification.

Step 4: (2S,4R)-4-((4-methoxybenzvl)(methyl)amino)pentan-2-ol

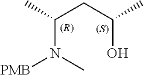

(2S,4R)-4-((4-methoxybenzyl)(methyl)amino)pentan-2-yl hydrogen sulfate (240 g, crude) and 2M H₂SO₄ in THF (3.4 L)/H₂O (0.34 L) was stirred for 15 min at 0° C. The resulting mixture was stirred for additional 2 h at 50° C. The mixture was basified to pH >7 with NaHICO3(aq) and extracted with EtOAc (3×2 L). The combined organic layers were washed with brine (2 L), dried over anhydrous Na₂SO₄. After filtration, the filtrate was concentrated under reduced pressure. The residue was purified by silica gel column chromatography, eluted with CH Cl₂/MeOH (100:0˜90:10) to give title product (75 g). MS (ESI, m c) [M+H]⁺ 238.25.

Step 5: (2S,4R)-4-(methylamino) pentan-2-ol hydrochloride

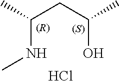

A solution of (2S,4R)-4-((4-methoxybenzyl)(methyl)amino)pentan-2-ol (20 g, 84 mmol) and Pd/C (20 g) in MeOH (200 mL) was stirred for 16 hrs at 50° C. The resulting mixture was filtered. A mixture of filtrate and 4M HCl in dioxane (100 mL) was stirred for 30 min at room temperature. The resulting mixture was concentrated under reduced pressure. The residue was dissolved by H₂O (200 mL) and extracted with EtOAc (3*300 mL). The aqueous phase was concentrated. This resulted in the title product (11 g, 85%). MS (ESI, m e) [M+H]⁺ 118.1.

Example 34 was prepared by a procedure similar to that described in Example 30 by replacing 2-(3-(methylamino)oxctan-3-yl)ethan-1-ol with (2S,4R)-4-(methylamino)pentan-2-ol hydrochloride to give the title product (23 mg). ¹H NMR (500 MHZ, CD₃OD) δ 6.94-6.79 (m, 1H), 6.70-6.38 (m, 1H), 5.46-5.23 (m, 1H), 4.63-4.50 (m, 1H), 4.44-4.24 (m, 2H), 3.76-3.57 (m, 1H), 3.44-3.33 (m, 3H), 3.27-3.21 (m, 3H), 3.13-3.05 (m, 1H), 2.41-1.77 (m, 8H), 1.53-1.47 (m, 3H), 1.35-1.29 (m, 3H). MS (ESI, m/e) [M+H]⁺ 613.2.

Example 35: 3-chloro-5-(11-ethyl-4-fluoro-2-(((2R,7aS)-2-fluorotetrahydro-1H-pyrrolizin-7a(5H)-yl)methoxy)-8,9-dihydro-11H-7-oxa-1,3,6,11-tetraazaspiro[cycloocta[de]naphthalene-10,3′-oxetan]-1(11a), 2,3a, 3a¹(6a), 5-pentaen-5-yl)-4-(trifluoromethyl)aniline

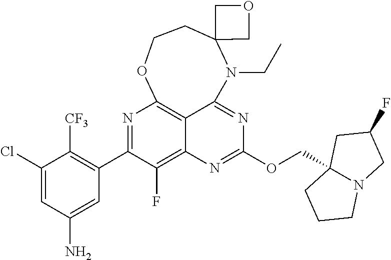 [SMILES: CCN1c2nc(OC[C@@]34CCCN3C[C@H](F)C4)nc3c(F)c(-c4cc(N)cc(Cl)c4C(F)(F)F)nc(c23)OCCC12COC2]

Example 35 was prepared by a procedure similar to that described in Example 30 by replacing 2-(3-(methylamino)oxetan-3-yl)ethan-1-ol with 2-(3-(ethylamino)oxetan-3-yl)ethan-1-ol to give the title product (32 mg). ¹H NMR (500 MHZ, CD₃OD) δ 6.90-6.83 (m, 1H), 6.58-6.46 (m, 1H), 5.42-5.23 (m, 1H), 4.71-4.46 (m, 5H), 4.42-4.17 (m, 3H), 3.76-3.60 (m, 2H), 3.10-3.01 (m, 1H), 2.43-1.87 (m, 7H), 1.54-1.46 (m, 3H). MS (ESI, m/e) [M+H]⁺ 641.2.

Example 36: 3-chloro-5-(4′-fluoro-2′-(((2R,7aS)-2-fluorotetrahydro-1H-pyrrolizin-7a(5H)-yl)methoxy)-10′-(2-(methylamino)ethyl)-8′H, 10′H-7′-oxa-1′,3′,6′,10′-tetraazaspiro[cyclobutane-1,9′-cyclohepta[de]naphthalen]-1′(10a′), 2′0.3a′, 3 al′(6a′), 5′-pentaen-5′-yl)-4-(trifluoromethyl)aniline

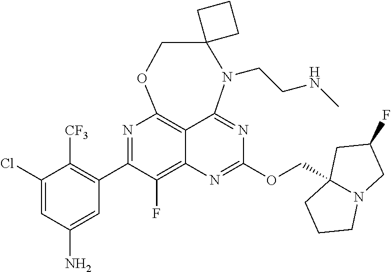 [SMILES: CNCCN1c2nc(OC[C@@]34CCCN3C[C@H](F)C4)nc3c(F)c(-c4cc(N)cc(Cl)c4C(F)(F)F)nc(c23)OCC12CCC2]

Example 36 was prepared by a procedure similar to that described in Example 20 by replacing tert-butyl (2-((4-hydroxybutan-2-yl)amino)ethyl)(methyl)carbamate with tert-butyl (2-((1-(hydroxymethyl)cyclobutyl)amino)ethyl)(methyl)carbamate to give the title product (2.0 mg). ¹H NMR (500 MHZ, CD₃OD) δ6.89 (s, 1H), 6.48 (s, 1H), 5.42-5.22 (m, 1H), 4.75-4.60 (m, 2H), 4.37-4.21 (m, 4H), 3.34-3.31 (m, 4H), 3.11-3.07 (m, 1H), 2.77 (s, 3H), 2.62-2.58 (m, 2H), 2.33-2.18 (m, 5H), 2.04-1.91 (m, 5H). MS (ESI, m/e) [M+H]⁺ 654.5.

Example 37: 3-chloro-5-(10′-(2-(dimethylamino)ethyl)-4′-fluoro-2′-(((2R,7aS)-2-fluorotetrahydro-1H-pyrrolizin-7a(5H)-yl)methoxy)-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaen-5′-yl)-4-(trifluoromethyl)aniline

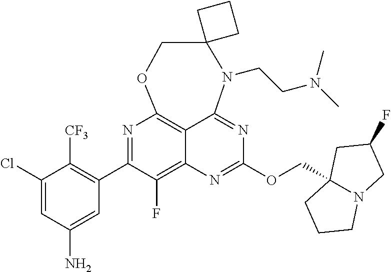 [SMILES: CN(C)CCN1c2nc(OC[C@@]34CCCN3C[C@H](F)C4)nc3c(F)c(-c4cc(N)cc(Cl)c4C(F)(F)F)nc(c23)OCC12CCC2]

Example 37 was prepared by a procedure similar to that described in Example 36 by replacing tert-butyl (2-((1-(hydroxymethyl)cyclobutyl)amino)ethyl)(methyl)carbamate with (1-((2-(dimethylamino)ethyl)amino)cyclobutyl)methanol to give the title product (0.5 mg). 6.88 (s, 1H), 6.48 (s, 1H), 5.45-5.25 (m, 1H), 4.66-4.65 (m, 2H), 4.34-4.32 (m, 2H), 4.17-4.15 (m, 2H), 2.85-2.83 (m, 2H), 2.62-2.58 (m, 2H), 2.50 (s, 6H), 2.33-2.01 (m, 10H). MS (ESI, m/e) [M+H]⁺ 668.5.

Example 38: 3-chloro-5-(4′-fluoro-2′-(((2R,7aS)-2-fluorotetrahydro-1H-pyrrolizin-7a(5H)-yl)methoxy)-10′-(1-(methylamino)propan-2-yl)-8′H, 10′H-7′-oxa-1′,3′,6′,10′-tetraazaspiro[cyclobutane-1,9′-cyclohepta[de]naphthalen]-1′(10a′), 2′,3a′, 3 al′(6a′), 5′-pentaen-5′-yl)-4-(trifluoromethyl)aniline

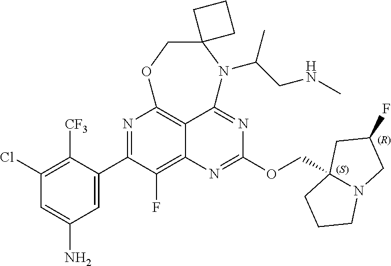

Step 1: tert-butyl (2-((1-(hydroxymethyl)cyclobutyl)amino)propyl)carbamate

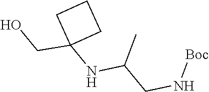 [SMILES: CC(CNC(=O)OC(C)(C)C)NC1(CO)CCC1]

To a mixture of 1-aminocyclobutyl)methanol, hydrochloride salt (1.37 mg, 10 mmol) and tert-butyl (2-oxopropyl)carbamate (1.73 g, 10 mmol) in THF (20 mL) and HOAc (2 mL) was added NaBH(OAc)₃ (4.24 g. 20 mmol). The mixture was stirred for 15 hours at 20° C. The cooled resulting mixture was concentrated. The residue was purified by column chromatography (DCM/MeOH=10/1) to give the product (2,2 g). MS (ESI, m e) [M+H]⁺ 259.4.

Example 38 was prepared by a procedure similar to that described in Example 20 by replacing tert-butyl (2-((4-hydroxybutan-2-yl)amino)ethyl)(methyl)carbamate with tert-butyl (2-((1-(hydroxymethyl)cyclobutyl)amino)propyl)carbamate to give the title product (5.2 mg). ¹H NMR (500 MHz, CD₃OD) δ6.89 (s, 1H), 6.47 (s, 1H), 5.41-5.21 (m, 1H), 4.58-4.44 (m, 3H), 4.39-4.23 (m, 3H), 3.76-3.69 (m, 1H), 3.49-3.30 (m, 4H), 2.75 (s, 3H), 2.51-1.91 (m, 12H), 1.65-1.64 (m, 3H). MS (ESI, m/e) [M+H]⁺ 668.5.

Example 39: 3-chloro-5-(4-fluoro-2-(((2R,7aS)-2-fluorotetrahydro-1H-pyrrolizin-7a(5H)-yl)methoxy)-11-(1-(methylamino)propan-2-yl)-8,9,10,11-tetrahydro-7-oxa-1,3,6,11-tetraazacycloocta[de]naphthalen-5-yl)-4-(trifluoromethyl)aniline

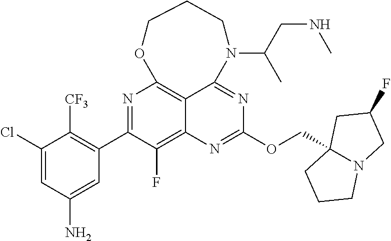 [SMILES: CNCC(C)N1CCCOc2nc(-c3cc(N)cc(Cl)c3C(F)(F)F)c(F)c3nc(OC[C@@]45CCCN4C[C@H](F)C5)nc1c23]

Example 39 was prepared by a procedure similar to that described in Example 20 by replacing tert-butyl (2-((4-hydroxybutan-2-yl)amino)ethyl)(methyl)carbamate with tert-butyl (2-((3-hydroxypropyl)amino)propyl)(methyl)carbamate to give the title product (2.5 mg). ¹H NMR (500 MHZ, DMSO-d₆) ¿ 6.85 (s, 1H), 6.47 (s, 1H), 6.30 (s, 2H), 5.38-5.17 (m, 1H), 4.45-4.32 (m, 2H), 4.12-3.99 (m, 2H), 3.58-3.47 (m, 2H), 3.19-2.99 (m, 5H), 2.85-2.82 (m, 2H), 2.39 (s, 3H), 2.12-1.77 (m, 8H), 1.34-1.33 (m, 3H). MS (ESI, m/e) [M+H]⁺ 642.6.

Example 40: 3-chloro-5-(11-(1-(dimethylamino)propan-2-yl)-4-fluoro-2-(((2R,7aS)-2-fluorotetrahydro-1H-pyrrolizin-7a(5H)-yl)methoxy)-8,9,10,11-tetrahydro-7-oxa-1,3,6,11-tetraazacycloocta[de]naphthalen-5-yl)-4-(trifluoromethyl)aniline

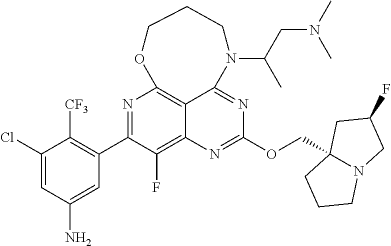 [SMILES: CC(CN(C)C)N1CCCOc2nc(-c3cc(N)cc(Cl)c3C(F)(F)F)c(F)c3nc(OC[C@@]45CCCN4C[C@H](F)C5)nc1c23]

Example 40 was prepared by a procedure similar to that described in Example 39 by replacing tert-butyl (2-((3-hydroxypropyl)amino)propyl)(methyl)carbamate with 3-((1-(dimethylamino)propan-2-yl)amino)propan-1-ol to give the title product (0.9 mg). ¹H NMR (500 MHZ, CD₃OD) δ6.99-6.88 (m, 1H), 6.62-6.50 (m, 1H), 5.45-5.24 (m, 1H), 4.52-4.23 (m, 4H), 3.67-3.60 (m, 2H), 3.34-3.31 (m, 3H), 3.09-2.92 (m, 2H), 2.18-1.91 (m, 6H), 1.31-1.29 (m, 3H). MS (ESI, m/e) [M+H]⁺ 656.4.

Example 41: 3-chloro-5-(4-fluoro-2-(((2R,7aS)-2-fluorotetrahydro-1H-pyrrolizin-7a(5H)-yl)methoxy)-11-((R)-pyrrolidin-3-yl)-8,9,10,11-tetrahydro-7-oxa-1,3,6,11-tetraazacycloocta[de]naphthalen-5-yl)-4-(trifluoromethyl)aniline

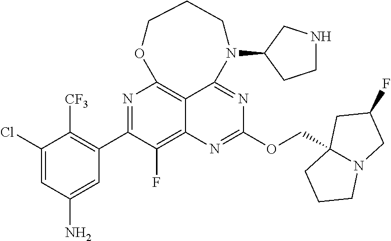 [SMILES: Nc1cc(Cl)c(C(F)(F)F)c(-c2nc3c4c(nc(OC[C@@]56CCCN5C[C@H](F)C6)nc4c2F)N([C@@H]2CCNC2)CCCO3)c1]

Example 41 was prepared by a procedure similar to that described in Example 20 by replacing tert-butyl (2-((4-hydroxybutan-2-yl)amino)ethyl)(methyl)carbamate with tert-butyl (R)-3-((3-hydroxypropyl)amino)pyrrolidine-1-carboxylate to give the title product (8.1 mg). MS (ESI, m/e) [M+H]⁺ 641.0.

Example 42: 3-chloro-5-(4-fluoro-2-(((2R,7aS)-2-fluorotetrahydro-1H-pyrrolizin-7a(5H)-yl)methoxy)-11-(2-(methylamino)ethyl)-8,9-dihydro-11H-7-oxa-1,3,6,11-tetraazaspiro[cycloocta[de]naphthalene-10,1′-cyclopropan]-1(11a), 2,3a, 4,6-pentaen-5-yl)-4-(trifluoromethyl)aniline

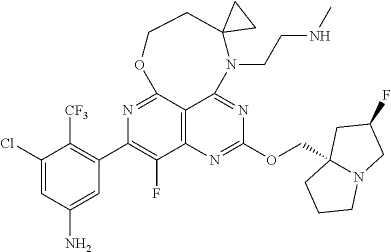 [SMILES: CNCCN1c2nc(OC[C@@]34CCCN3C[C@H](F)C4)nc3c(F)c(-c4cc(N)cc(Cl)c4C(F)(F)F)nc(c23)OCCC12CC2]

Example 42 was prepared by a procedure similar to that described in Example 20 by replacing tert-butyl (2-((4-hydroxybutan-2-yl)amino)ethyl)(methyl)carbamate with tert-butyl (2-((1-(2-hydroxyethyl)cyclopropyl)amino)ethyl)(methyl)carbamate to give the title product (20 mg). ¹H NMR (500 MHz, CD₃OD) δ6.89 (s, 1H), 6.64-6.38 (m, 1H), 5.50-5.30 (m, 1H), 4.58-4.47 (m, 4H), 4.12 (s, 2H), 3.52-3.41 (m, 5H), 3.22-3.16 (m, 1H), 2.79 (s, 3H), 2.65-1.95 (m, 7H), 1.68-1.58 (m, 1H), 1.11-0.61 (m, 4H). MS (ESI, m/e) [M+H]⁺ 654.3.

Example 43: 3-chloro-5-(11-(2-(dimethylamino)ethyl)-4-fluoro-2-(((2R,7aS)-2-fluorotetrahydro-1H-pyrrolizin-7a(5H)-yl)methoxy)-8,9-dihydro-11H-7-oxa-1,3,6,11-tetraazaspiro[cycloocta[de]naphthalene-10,1′-cyclopropan]-1(11a), 2,3a, 4,6-pentaen-5-yl)-4-(trifluoromethyl)aniline

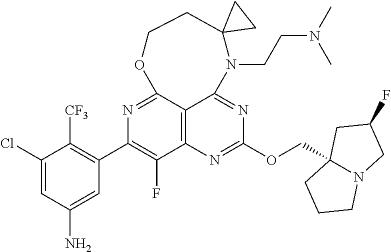 [SMILES: CN(C)CCN1c2nc(OC[C@@]34CCCN3C[C@H](F)C4)nc3c(F)c(-c4cc(N)cc(Cl)c4C(F)(F)F)nc(c23)OCCC12CC2]

Example 43 was prepared by a procedure similar to that described in Example 42 by replacing tert-butyl (2-((1-(2-hydroxyethyl)cyclopropyl)amino)ethyl)(methyl)carbamate with 2-(1-((2-(dimethylamino)ethyl)amino)cyclopropyl)ethan-1-ol to give the title product (20 mg). ¹H NMR (500 MHz, CD₃OD) δ 7.02-6.89 (m, 1H), 6.64-6.38 (m, 1H), 5.54-5.33 (m, 1H), 4.57-4.44 (m, 4H), 4.14-3.96 (m, 2H), 3.69-3.53 (m, 3H), 3.16-3.06 (m, 2H), 2.64-2.62 (m, 6H), 2.50-2.37 (m, 2H), 2.30-2.15 (m, 3H), 2.07-1.98 (m, 1H), 1.67-1.58 (s, 1H), 1.12-1.57 (m, 4H). MS (ESI, m/e) [M+H]⁺ 668.3.

Example 44: 3-chloro-5-(11-(2-(dimethylamino)ethyl)-4-fluoro-2-(((2R,7aS)-2-fluorotetrahydro-1H-pyrrolizin-7a(5H)-yl)methoxy)-8,9-dihydro-11H-7-oxa-1,3,6,11-tetraazaspiro[cycloocta[de]naphthalene-10,3′-oxetan]-1(11a), 2,3a, 4,6-pentaen-5-yl)-4-(trifluoromethyl)aniline

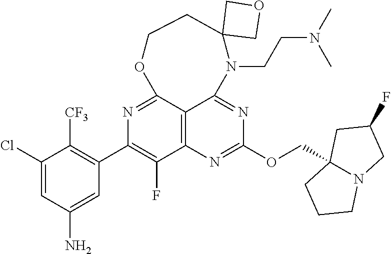 [SMILES: CN(C)CCN1c2nc(OC[C@@]34CCCN3C[C@H](F)C4)nc3c(F)c(-c4cc(N)cc(Cl)c4C(F)(F)F)nc(c23)OCCC12COC2]

Example 44 was prepared by a procedure similar to that described in Example 20 by replacing tert-butyl (2-((4-hydroxybutan-2-yl)amino)ethyl)(methyl)carbamate with 2-(3-((2-(dimethylamino)ethyl)amino)oxetan-3-yl)ethan-1-ol to give the title product (2 mg). ¹H NMR (500 MHZ, CD₃OD) δ7.03-6.89 (m, 1H), 6.63-6.51 (m, 1H), 5.62-5.43 (m, 1H), 4.70-4.55 (m, 7H), 3.91-3.77 (m, 5H), 3.49-3.40 (m, 4H), 2.89-2.80 (m, 6H), 2.65-2.10 (m, 7H). MS (ESI, m/e) [M+H]⁺ 684.3.

Example 45: 3-chloro-5-((7a, 10a-trans)-4-fluoro-2-(((2R,7aS)-2-fluorotetrahydro-1H-pyrrolizin-7a(5H)-yl)methoxy)-11-methyl-7a, 8,9,10,10a, 11-hexahydro-7-oxa-1,3,6,11-tetraazanaphtho[1,8-fg]azulen-5-yl)-4-(trifluoromethyl)aniline

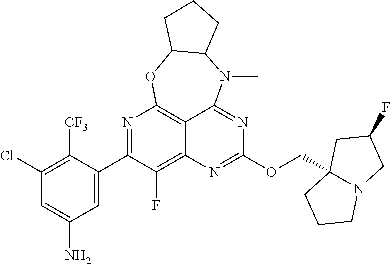 [SMILES: CN1c2nc(OC[C@@]34CCCN3C[C@H](F)C4)nc3c(F)c(-c4cc(N)cc(Cl)c4C(F)(F)F)nc(c23)OC2CCCC21]

Step 1: 1,2-trans -2-(methylamino)cyclopentan-1-ol

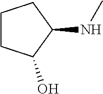

To a solution of tert-butyl (trans-2-hydroxycyclopentyl)carbamate (1 g, 4.97 mmol) in THF(100 mL) was added LiAlH₄ (400 mg, 9.3 mmol), and the mixture was stirred at 60° C. overnight. After completion, the mixture was quenched by Na₂SO₄. 10 H₂O. Then filtered and the filtrate was concentrated to give the title product (500 mg). MS (ESI, m/e) [M+H]⁺ 116.2.

Example 45 was prepared by a procedure similar to that described in Example 24 by replacing (1-(methylamino) cyclobutyl) methanol with 1,2-trans -2-(methylamino)cyclopentan-1-ol to give the title product (18 mg). ¹H NMR (500 MHZ, DMSO-d₆) δ6.85 (s, 1H), 6.54-6.30 (m, 3H), 5.41-5.21 (m, 1H), 4.78-4.72 (m, 1H), 4.25-4.00 (m, 3H), 3.26-3.07 (m, 6H), 2.93-2.84 (m, 1H), 2.40-2,24 (m, 2H), 2.30-1.67 (m, 10H). MS (ESI, m/e) [M+H]⁺ 611.4.

Example 46: 3-chloro-5-((7aS,11aS)-4-fluoro-2-(((2R,7aS)-2-fluorotetrahydro-1H-pyrrolizin-7a(5H)-yl) methoxy)-12-methyl-7a, 8,10,11,11a, 12-hexahydro-9H-7-oxa-1,3,6,12-tetraazapleiaden-5-yl)-4-(trifluoromethyl)aniline

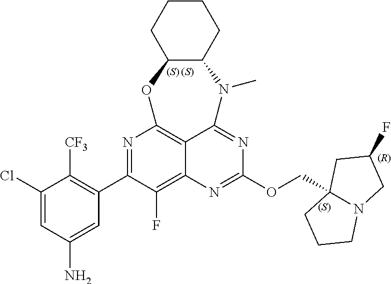

Step 1: (1S,2S)-2-(methylamino) cyclohexan-1-ol

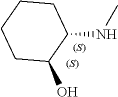

To a solution of tert-butyl ((1S,2S)-2-hydroxycyclohexyl) carbamate (1 g, 4.65 mmol) in THF (25 mL) was added LiAlH₄ (530 mg, 13.95 mmol) at 0° C., and the mixture was stirred at 70° C. overnight. Upon completion, the mixture was cooled to room temperature, sodium sulfate decahydrate was added and stirred for 0.5 h. The resulting mixture was filtered, and the organic phase was concentrated to give a residue as the title compound (630 mg crude). MS (ESI, m/e) [M+H]⁺ 130.1.

Example 46 was prepared by a procedure similar to that described in Example 24 by replacing (1-(methylamino) cyclobutyl) methanol with (1S,2S)-2-(methylamino) cyclohexan-1-ol to give the title product (6.1 mg). ¹H NMR (500 MHZ, CD₃OD) δ6.90-6.86 (m, 1H), 6.58-6.37 (m, 1H), 5.46-6.25 (m, 1H), 4.39-4.26 (m, 3H), 3.90-3.70 (m, 1H), 3.49-3.31 (m, 3H), 3.14-3.06 (m, 1H), 2.58-2.56 (m, 1H), 2.44-1.68 (m, 10H), 1.46-1.26 (m, 3H). MS (ESI, m/e) [M+H]⁺ 625.2.

Example 47: (7aR*,11S*,11aR*)-5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-12-methyl-7a, 8,10,11,11a, 12-hexahydro-9H-7-oxa-1,3,6,12-tetraazapleiaden-11-ol

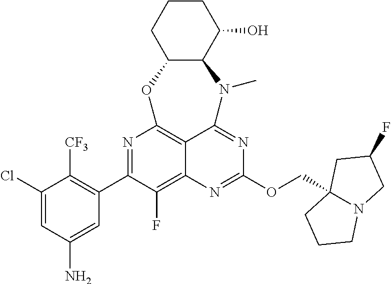 [SMILES: CN1c2nc(OC[C@@]34CCCN3C[C@H](F)C4)nc3c(F)c(-c4cc(N)cc(Cl)c4C(F)(F)F)nc(c23)O[C@@H]2CCC[C@H](O)[C@H]21]

Step 1: 5-(((1R⁸. 2R⁸. 3S*)-2-amino-3-hydroxycyclohexyl)oxy)-7-chloro-8-fluoro-2-(methylthio)pyrido[4,3-d]pyrimidin-4-ol

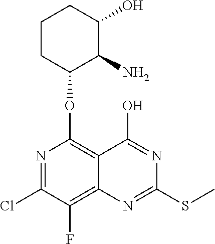 [SMILES: CSc1nc(O)c2c(O[C@@H]3CCC[C@H](O)[C@H]3N)nc(Cl)c(F)c2n1]

To a stirred solution of 5,7-dichloro-8-fluoro-2-(methylthio)pyrido[4,3-d]pyrimidin-4-ol (560 mg, 2.0 mmol) and (1R*, 2r*,3S*)-2-aminocyclohexane-1,3-diol (CAS #38332-12-6, Batch #22102304 from Shanghai Topbiochem Technology, relative stereochemistry, 265 mg, 2.0 mmol) in THF (20 mL) was added NaH (320 mg, 8.0 mmol, 60%) at 0° C., the resulting mixture was stirred for 1 h at room temperature. The reaction mixture was quenched with H₂O, then adjusted pH to 6 with 1N aq HCl. The mixture was filtered and filter cake was directly used in the next step without further purification. MS (ESI, m/e) [M+H]⁺ 375.2.

Step 2: (7aR*,11S*,11aR*)-5-chloro-4-fluoro-2-(methylthio)-7a, 8,10,11,11a, 12-hexahydro-9H-7-oxa-1,3,6,12-tetraazapleiaden-11-ol

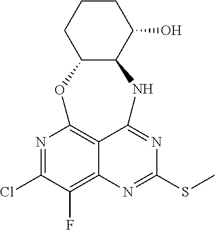 [SMILES: CSc1nc2c3c(nc(Cl)c(F)c3n1)O[C@@H]1CCC[C@H](O)[C@H]1N2]

To a stirred solution of 5-(((1R*,2R*,3S*)-2-amino-3-hydroxycyclohexyl)oxy)-7-chloro-8-fluoro-2-(methylthio)pyrido[4,3-d]pyrimidin-4-ol (2.0 mmol) in MeCN (100 mL) was added DIEA (774 mg, 6.0 mmol) and BOPCl (1.0 g, 4.0 mmol) at room temperature, the resulting mixture was stirred for 1 h at 70° C. The reaction mixture was concentrated and purified by flash chromatography (DCM/EtOAc=5:1 to 3:1) to afford the desired product (250 mg). MS (ESI, m/e) [M+H]⁺ 357.1.

Step 3: (7aR*,11S*,11aS*)-11-((tert-butyldimethylsilyl)oxy)-5-chloro-4-fluoro-2-(methylthio)-7a, 8,10,11,11a, 12-hexahydro-9H-7-oxa-1,3,6,12- tetraazapleiadene

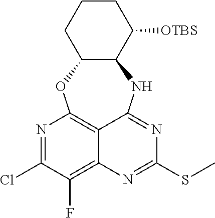 [SMILES: CSc1nc2c3c(nc(Cl)c(F)c3n1)O[C@@H]1CCC[C@H](O[Si](C)(C)C(C)(C)C)[C@H]1N2]

To a stirred solution of (7aR*,11S*,11aR*)-5-chloro-4-fluoro-2-(methylthio)-7a, 8,10,11,11a, 12-hexahydro-9H-7-oxa-1,3,6,12-tetraazapleiaden-11-ol (250 mg, 0.7 mmol) in DCM (10 mL) was added DIEA (290 mg, 1,1 mmol) and TBSOTf (180 mg, 1.4 mmol) at 0° C., and the resulting mixture was stirred at room temperature for 1 h. The reaction mixture was concentrated and purified by flash chromatography (PE/EtOAc=5:1 to 1:1) to afford the desired product (138 mg). MS (ESI, m e) [M+H]⁺ 471.2.

Step 4: (7aR*,11S*,11aS*)-11-((tert-butyldimethylsilyl)oxy)-5-chloro-4-fluoro-12-methyl-2-(methylthio)-7a, 8,10,11,11a, 12-hexahydro-9H-7-oxa-1,3,6,12-tetraazapleiadene

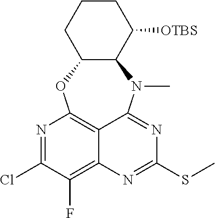 [SMILES: CSc1nc2c3c(nc(Cl)c(F)c3n1)O[C@@H]1CCC[C@H](O[Si](C)(C)C(C)(C)C)[C@H]1N2C]

To a stirred solution of (7aR*,11S*,11aS*)-11-((tert-butyldimethylsilyl)oxy)-5-chloro-4-fluoro-2-(methylthio)-7a, 8,10,11,11a, 12-hexahydro-9H-7-oxa-1,3,6,12-tetraazapleiadene (138 mg, 0.3 mmol) in THF (6 mL) was added NaH (36 mg, 0.9 mmol, 60%) at 0° C., and the resulting mixture was stirred at 0° C. for 15 min. Then, to the above mixture was added CH₃I (128 mg, 0.9 mmol), and the reaction mixture was stirred for 3 hrs at 50° C. The reaction was quenched with water, the crude was purified by flash chromatography (PE/EtOAc=5:1 to 1:1) to afford the desired product (132 mg). MS (ESI, m/e) [M+H]⁺ 485.3.

Step 5: (7aR*,11S*,11aS*)-11-((tert-butyldimethylsilyl)oxy)-5-chloro-4-fluoro-12-methyl-2-(methylsulfinyl)-7a, 8,10,11,11a, 12-hexahydro-9H-7-oxa-1,3,6,12-tetraazapleiadene

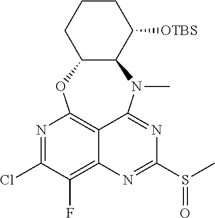 [SMILES: CN1c2nc(S(C)=O)nc3c(F)c(Cl)nc(c23)O[C@@H]2CCC[C@H](O[Si](C)(C)C(C)(C)C)[C@H]21]

To a stirred solution of (7aR*,11S*,11aS*)-11-((tert-butyldimethylsilyl)oxy)-5-chloro-4-fluoro-12-methyl-2-(methylthio)-7a, 8,10,11,11a, 12-hexahydro-9H-7-oxa-1,3,6,12-tetraazapleiadene (132 mg, 0.27 mmol) in DCM (4 mL) was added m-CPBA (55 mg, 0.27 mmol, 85%) at 0° C., the resulting mixture was stirred at 0° C. for 15 min. The reaction was quenched with aq. Na₂S₂O₃, then extracted with DCM. The organic layer was washed with Sat, aq NaHCO₃ and brine, dried over anhydrous Na₂SO₄, concentrated in vacuo. The residue was directly used in the next step without further purification.

Step 6: (7aR*,11S*,11aS*)-11-((tert-butyldimethylsilyl)oxy)-5-chloro-4-fluoro-2-(((2R,7aS)-2-fluorotetrahydro-1H-pyrrolizin-7a(5H)-yl)methoxy)-12-methyl-7a, 8,10,11,11a, 12-hexahydro-9H-7-oxa-1,3,6,12-tetraazapleiadene

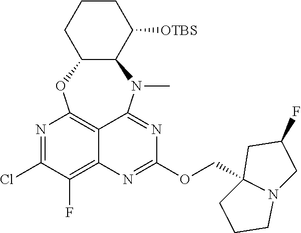 [SMILES: CN1c2nc(OC[C@@]34CCCN3C[C@H](F)C4)nc3c(F)c(Cl)nc(c23)O[C@@H]2CCC[C@H](O[Si](C)(C)C(C)(C)C)[C@H]21]

To a stirred solution of (7aR*, 11S*,11aS*)-11-((tert-butyldimethylsilyl)oxy)-5-chloro-4-fluoro-12-methyl-2-(methylsulfinyl)-7a, 8,10,11,11a, 12-hexahydro-9H-7-oxa-1,3,6,12-tetraazapleiadene (0.27 mmol) and ((2R,7aS)-2-fluorotetrahydro-1H-pyrrolizin-7a(5H)-yl)methanol (86 mg, 0.54 mmol) in THF (6 mL) was added LiHMDS (0.54 mL, 0.54 mmol, 1M in THF) dropwise at 0° C., and the resulting mixture was stirred at 0° C. for 20 min. The reaction was quenched with Sat, aq NH₄Cl, then extracted with DCM. The organic layer was washed with brine, dried over anhydrous Na₂SO₄, and concentrated in vacuo. The residue was purified by flash chromatography (DCM/MeOH=100:1 to 30:1) to afford the product (75 mg). MS (ESI, m/e) [M+H]⁺ 596.4.

Step 7: 3-((7aR*,11S*,11aS*)-11-((tert-butyldimethylsilyl)oxy)-4-fluoro-2-(((2R,7aS)-2-fluorotetrahydro-1H-pyrrolizin-7a(5H)-yl)methoxy)-12-methyl-7a, 8,10,11,11a, 12-hexahydro-9H-7-oxa-1,3,6,12-tetraazapleiaden-5-yl)-5-chloro-4-(trifluoromethyl)aniline

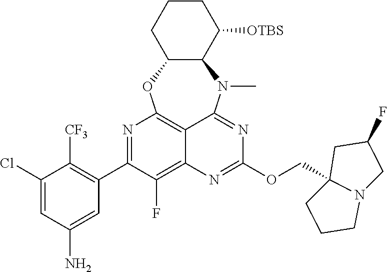 [SMILES: CN1c2nc(OC[C@@]34CCCN3C[C@H](F)C4)nc3c(F)c(-c4cc(N)cc(Cl)c4C(F)(F)F)nc(c23)O[C@@H]2CCC[C@H](O[Si](C)(C)C(C)(C)C)[C@H]21]

To a mixture of (7aR*,11S*,11aS*)-11-((tert-butyldimethylsilyl)oxy)-5-chloro-4-fluoro-2-(((2R,7aS)-2-fluorotetrahydro-1H-pyrrolizin-7a(5H)-yl)methoxy)-12-methyl-7a, 8,10,11,11a, 12-hexahydro-9H-7-oxa-1,3,6,12-tetraazapleiadene (75 mg, 0.12 mmol), 3-chloro-5-(4,4,5,5-tetramethyl-1,3,2-dioxaborolan-2-yl)-4-(trifluoromethyl)aniline (58 mg, 0.18 mmol), Pd(dtbpf)Cl₂ (15 mg, 0.02 mmol) and NaHCO₃ (30 mg, 0.36 mmol) was added dioxane (4.0 mL) and H₂O (0.8 mL), and the resulting mixture was stirred at 90° C. for 1 h. The reaction mixture was concentrated and purified by flash chromatography (DCM/MeOH=100:1 to 5:1) to give the product (68 mg). MS (ESI, m/e) [M+H]⁺ 755.5.

Step 8: (7aR*,11S*,11aR*)-5-(5-amino-3-chloro-2-(trifluoromethyl)phenyl)-4-fluoro-2-(((2R,7aS)-2-fluorotetrahydro-1H-pyrrolizin-7a(5H)-yl)methoxy)-12-methyl-7a, 8,10,11,11a, 12-hexahydro-9H-7-oxa-1,3,6,12-tetraazapleiaden-11-ol

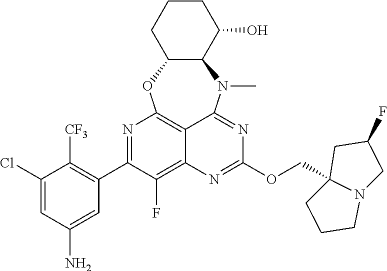 [SMILES: CN1c2nc(OC[C@@]34CCCN3C[C@H](F)C4)nc3c(F)c(-c4cc(N)cc(Cl)c4C(F)(F)F)nc(c23)O[C@@H]2CCC[C@H](O)[C@H]21]

To a mixture of 3-((7aR*,11S*,11aS*)-11-((tert-butyldimethylsilyl)oxy)-4-fluoro-2-(((2R,7aS)-2-fluorotetrahydro-1H-pyrrolizin-7a(5H)-yl)methoxy)-12-methyl-7a, 8,10,11,11a, 12-hexahydro-9H-7-oxa-1,3,6,12-tetraazapleiaden-5-yl)-5-chloro-4-(trifluoromethyl)aniline (68 mg, 0.09 mmol) in DCM (3.0 mL) was 4N HCl(in dioxane) (3.0 mL). The resulting mixture was stirred for 20 min. The reaction mixture was concentrated and based with DIEA, then prep-HPLC to give the product (19.5 mg). ¹H NMR (500 MHZ, CD₃OD) δ 6.92-6.77 (m, 1H), 6.60-6.37 (m, 1H), 5.44-5.19 (m, 1H), 4.80-4.51 (m, 2H), 4.45-4.12 (m, 2H), 4.09-3.85 (m, 1H), 3.53 (s, 3H), 3.46-3.35 (m, 1H), 3.27-3.12 (m. 2H), 3.11-2.90 (m, 1H), 2.50-1.20 (m, 12H). MS (ESI, m/e) [M+H]⁺ 641.3.

Example 48: 3-chloro-5-((7aS,11aS)-4-fluoro-2-(((2R,7aS)-2-fluorotetrahydro-1H-pyrrolizin-7a(5H)-yl)methoxy)-12-(2-(methylamino)ethyl)-7a, 8,10,11,11a, 12-hexahydro-9H-7-oxa-1,3,6,12-tetraazapleiaden-5-yl)-4-(trifluoromethyl)aniline

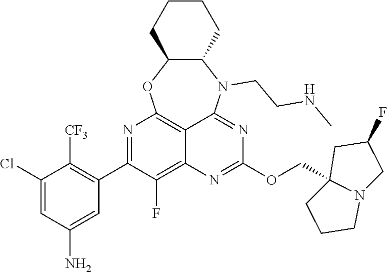 [SMILES: CNCCN1c2nc(OC[C@@]34CCCN3C[C@H](F)C4)nc3c(F)c(-c4cc(N)cc(Cl)c4C(F)(F)F)nc(c23)O[C@H]2CCCC[C@@H]21]

Example 48 was prepared by a procedure similar to that described in Example 20 by replacing tert-butyl (2-((4-hydroxybutan-2-yl)amino)ethyl)(methyl)carbamate with tert-butyl ((1S,2S)-2-hydroxycyclohexyl)carbamate to give the title product (2,2 mg). ¹H NMR (500 MHZ, CD₃OD) δ6.89 (s, 1H), 6.54-6.38 (m, 1H), 5.44-5.25 (m, 1H), 4.38-4.32 (m, 3H), 4.25-4.16 (m, 1H), 3.94-3.86 (m, 2H), 3.42-3.33 (m, 5H), 3.12-3.08 (m, 1H), 2.76 (s, 3H), 2.34-2.18 (m, 5H), 2.07-2.03 (m, 2H), 1.95-1.73 (m, 4H), 1.42-1.39 (m, 3H). MS (ESI, m/e) [M+H]⁺ 668.5.

Example 49: 3-chloro-5-((7aR,11aS)-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiaden-5-yl)-4-(trifluoromethyl)aniline

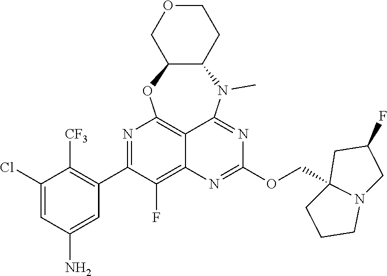 [SMILES: CN1c2nc(OC[C@@]34CCCN3C[C@H](F)C4)nc3c(F)c(-c4cc(N)cc(Cl)c4C(F)(F)F)nc(c23)O[C@H]2COCC[C@@H]21]

Step 1: 5-(((3R,4S)-4-aminotetrahydro-2H-pyran-3-yl)oxy)-7-chloro-8-fluoro-2-(methylthio)pyrido[4,3-d]pyrimidin-4-ol

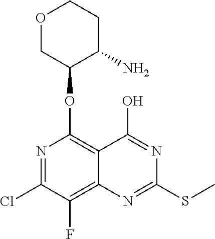 [SMILES: CSc1nc(O)c2c(O[C@H]3COCC[C@@H]3N)nc(Cl)c(F)c2n1]

To a stirred solution of 5,7-dichloro-8-fluoro-2-(methylthio)pyrido[4,3-d]pyrimidin-4-ol (280 mg, 1.0 mmol) and (3R,4S)-4-aminotetrahydro-2H-pyran-3-ol hydrochloride (154 mg, 1.0 mmol) in THF (10 mL) was added NaH (160 mg, 4.0 mmol, 60%) at 0° C., and the resulting mixture was stirred for 1 h at room temperature. The reaction mixture was quenched with H₂O, then adjusted pH to 6 with 1N aq HCl. The mixture was filtered and filter cake was directly used in the next step without further purification. MS (ESI, m/e) [M+H]⁺ 361,1.

Step 2: (7aR,11aS)-5-chloro-4-fluoro-2-(methylthio)-7a, 8,10,11,11a, 12-hexahydro-7,9-dioxa-1,3,6,12-tetraazapleiadene

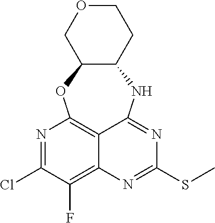 [SMILES: CSc1nc2c3c(nc(Cl)c(F)c3n1)O[C@H]1COCC[C@@H]1N2]

To a stirred solution of 5-(((3R,4S)-4-aminotetrahydro-2H-pyran-3-yl)oxy)-7-chloro-8-fluoro-2-(methylthio)pyrido[4,3-d]pyrimidin-4-ol (1.0 mmol) in ACN (50 mL) was added DIEA (387 mg, 3.0 mmol) and BOPCl (508 mg, 2.0 mmol) at room temperature, and the resulting mixture was stirred for 1 h at 70° C. The reaction mixture was concentrated and purified by flash chromatography (DCM EtOAc=5:1 to 3:1) to afford the desired product (75 mg). MS (ESI, m/e) [M+H]⁺ 343.1.

Step 3: (7aR,11aS)-5-chloro-4-fluoro-12-methyl-2-(methylthio)-7a, 8,10,11,11a, 12-hexahydro-7,9-dioxa-1,3,6,12-tetraazapleiadene

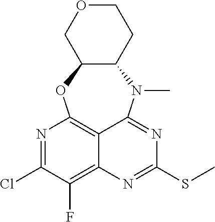 [SMILES: CSc1nc2c3c(nc(Cl)c(F)c3n1)O[C@H]1COCC[C@@H]1N2C]

To a stirred solution of (7aR,11aS)-5-chloro-4-fluoro-2-(methylthio)-7a, 8,10,11,11a, 12-hexahydro-7,9-dioxa-1,3,6,12-tetraazapleiadene (75 mg, 0.2 mmol) in THF (3 mL) was added NaH (24 mg, 0.6 mmol, 60%) at 0° C., the resulting mixture was stirred at 0° C. for 15 min. Then to the above mixture was added CH₃I (85 mg, 0.6 mmol), and the reaction mixture was stirred for 3 hrs at 50° C. The reaction was quenched with water, the crude was purified by flash chromatography (PE/EtOAc=5:1 to 1:1) to afford the desired product (74 mg). MS (ESI, m/e) [M+H]⁺ 357.1.

Step 4: (7aR,11aS)-5-chloro-4-fluoro-12-methyl-2-(methylsulfinyl)-7a, 8,10,11,11a, 12-hexahydro-7,9-dioxa-1,3,6,12-tetraazapleiadene

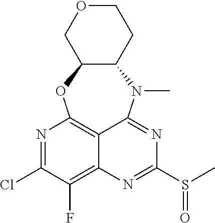 [SMILES: CN1c2nc(S(C)=O)nc3c(F)c(Cl)nc(c23)O[C@H]2COCC[C@@H]21]

To a stirred solution of (7aR,11aS)-5-chloro-4-fluoro-12-methyl-2-(methylthio)-7a, 8,10,11,11a, 12-hexahydro-7,9-dioxa-1,3,6,12-tetraazapleiadene (74 mg, 0.21 mmol) in DCM (3 mL) was added m-CPBA (43 mg, 0.21 mmol, 85%) at 0° C., and the resulting mixture was stirred at 0° C. for 15 min. The reaction was quenched with aq. Na₂S₂O₃, then extracted with DCM. The organic layer was washed with Sat, aq NaHCO₃ and brine, dried over anhydrous Na₂SO₄, concentrated in vacuo. The residue was directly used in the next step without further purification.

Step 5: (7aR,11aS)-5-chloro-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiadene

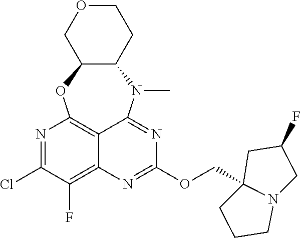 [SMILES: CN1c2nc(OC[C@@]34CCCN3C[C@H](F)C4)nc3c(F)c(Cl)nc(c23)O[C@H]2COCC[C@@H]21]

To a stirred solution of (7aR,11aS)-5-chloro-4-fluoro-12-methyl-2-(methylsulfinyl)-7a, 8,10,11,11a, 12-hexahydro-7,9-dioxa-1,3,6,12-tetraazapleiadene (0.21 mmol) and ((2R,7aS)-2-fluorotetrahydro-1H-pyrrolizin-7a(5H)-yl)methanol (222 mg, 0.42 mmol) in THF (8 mL) was added LiHMDS (0.4 mL, 0.42 mmol, 1M in THF) dropwise at 0° C., and the resulting mixture was stirred at 0° C. for 20 min. The reaction was quenched with Sat, aq NH₄Cl, then extracted with DCM. The organic layer was washed with brine, dried over anhydrous Na₂SO₄, and concentrated in vacuo. The residue was purified by flash chromatography (DCM/MeOH=100:1 to 30:1) to afford the product (53 mg). MS (ESI, m/e) [M+H]⁺ 468.3.

Step 6: 3-chloro-5-((7aR,11aS)-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiaden-5-yl)-4-(trifluoromethyl)aniline

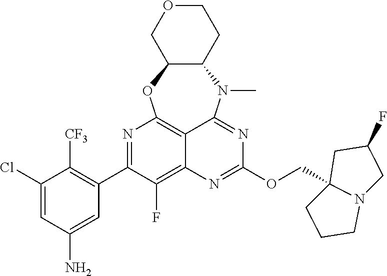 [SMILES: CN1c2nc(OC[C@@]34CCCN3C[C@H](F)C4)nc3c(F)c(-c4cc(N)cc(Cl)c4C(F)(F)F)nc(c23)O[C@H]2COCC[C@@H]21]

To a mixture of (7aR,11aS)-5-chloro-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiadene (27 mg, 0.06 mmol), 3-chloro-5-(4,4,5,5-tetramethyl-1,3,2-dioxaborolan-2-yl)-4-(trifluoromethyl)aniline (38 mg, 0.12 mmol), Pd(dtbpf)Cl₂ (8 mg, 0.01 mmol) and NaHCO₃ (15 mg, 0.18 mmol) was added dioxane (3.0 mL) and H₂O (0.6 mL), and the resulting mixture was stirred at 90° C. for 1 h. The reaction mixture was concentrated and purified by flash chromatography (DCM/MeOH=100:1 to 5:1), then prep-HPLC to give the product (7.9 mg). ¹H NMR (500 MHz, CD₃OD) & 6.92-6.83 (m, 1H), 6.62-6.30 (m, 1H), 5.42-5.20 (m, 1H), 4.46-4.17 (m, 4H), 4.11-3.95 (m, 2H), 3.54-3.43 (m, 2H), 3.40-3.32 (m, 3H), 3.29-3.14 (m, 2H), 3.11-2.97 (m, 1H), 2.53-1.63 (m, 8H). MS (ESI, m/e) [M+H]⁺ 627.3.

Example 50: 3-chloro-5-((7aS,11aS)-4-fluoro-2-(((2R,7aS)-2-fluorotetrahydro-1H-pyrrolizin-7a(5H)-yl) methoxy)-12-methyl-7a, 8,11a, 12-tetrahydro-9H, 11H-7,10-dioxa-1,3,6,12-tetraazapleiaden-5-yl)-4-(trifluoromethyl)aniline

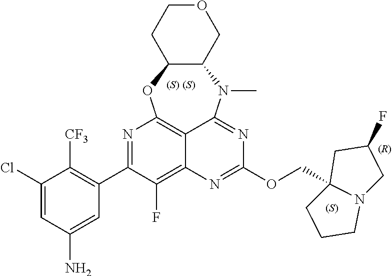

Example 50 was prepared by a procedure similar to that described in Example 49 by replacing (3R,4S)-4-aminotetrahydro-2H-pyran-3-ol hydrochloride with (3S,4S)-3-aminotetrahydro-2H-pyran-4-ol to give the title product (6.1 mg). ¹H NMR (500 MHZ, CD₃OD) δ6.92-6.83 (m, 1H), 6.56-6.39 (m, 1H), 5.43-5.23 (m, 1H), 4.59-4.44 (m, 3H), 4.38-4.26 (m, 3H), 4.08-3.88 (m, 3H), 3.48-3.36 (m, 2H), 3.10-3.03 (m, 2H), 2.32-1.89 (m, 10H). MS (ESI, m e) [M+H]⁺ 627.2.

Example 51: 3-chloro-5-((7aR,11aR)-4-fluoro-2-(((2R,7aS)-2-fluorotetrahydro-1H-pyrrolizin-7a(5H)-yl)methoxy)-12-methyl-7a, 8,11a, 12-tetrahydro-9H, 11H-7,10-dioxa-1,3,6,12-tetraazapleiaden-5-yl)-4-(trifluoromethyl)aniline

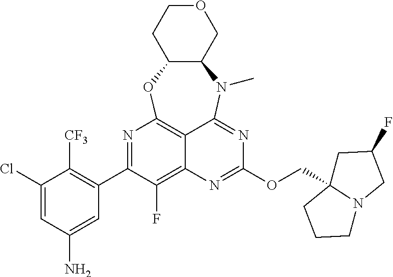 [SMILES: CN1c2nc(OC[C@@]34CCCN3C[C@H](F)C4)nc3c(F)c(-c4cc(N)cc(Cl)c4C(F)(F)F)nc(c23)O[C@@H]2CCOC[C@H]21]

Example 51 was prepared by a procedure similar to that described in Example 49 by replacing (3R,4S)-4-aminotetrahydro-2H-pyran-3-ol hydrochloride with (3R,4R)-3-aminotetrahydro-2H-pyran-4-ol hydrochloride to give the title product (15 mg). ¹H NMR (500 MHZ, CD₃OD) δ 6.92-6.83 (m, 1H), 6.60-6.32 (m, 1H), 5.49-5.23 (m, 1H), 4.62-4.26 (m, 4H), 4.10-3.86 (m, 2H), 3.55-3.34 (m, 4H), 3.27-3.19 (m, 1H), 3.16-3.05 (m, 1H), 2.47-1.84 (m, 8H). MS (ESI, m/e) [M+H]⁺ 627.3.

Example 52: 3-chloro-5-((8aS,11aR)-4-fluoro-2-(((2R,7aS)-2-fluorotetrahydro-1H-pyrrolizin-7a(5H)-yl)methoxy)-12-methyl-8a, 9,10,11,11a, 12-hexahydro-8H-7-oxa-1,3,6,12-tetraazacyclopenta[5,6]cycloocta[1,2,3-de]naphthalen-5-yl)-4-(trifluoromethyl)aniline

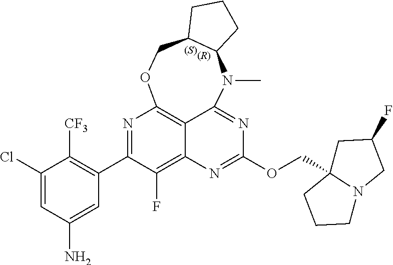

Step 1: ((1S,2R)-2-(methylamino)cyclopentyl)methanol

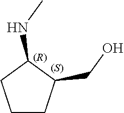

A solution of (1S,2R)-2-((tert-butoxycarbonyl)amino)cyclopentane-1-carboxylic acid (1 g, 4.4 mmol) in THF (20 mL) was treated with lithium aluminium hydride (1N in THF, 5.3 mL) at ambient temperature, followed by refluxing for 16 hrs. The mixture was cooled to 0-5° C., and carefully quenched with excess sodium sulfate decahydrate. Additional THF and diethyl ether were added to aid stirring. The mixture was filtered through Celite® and the filtrate was evaporated to give the product (0.44 g). MS (ESI, m/e) [M+H]⁺ 130.

Example 52 was prepared by a procedure similar to that described in Example 11 by replacing (3-((methylamino)methyl)oxetan-3-yl)methanol with ((1S,2R)-2-(methylamino)cyclopentyl)methanol to give the title product (5.9 mg). ¹H NMR (500 MHZ, CD₃OD) δ6.92-6.86 (m, 1H), 6.64-6.40 (m, 1H), 5.44-5.23 (m, 1H), 4.43-4.23 (m, 3H), 3.79-3.37 (m, 4H), 3.17-3.04 (m, 1H), 2.60-1.46 (m. 16H). MS (ESI, m/e) [M+H]⁺ 625.2.

Example 53: 3-(4-fluoro-2-(((2R,7aS)-2-fluorotetrahydro-1H-pyrrolizin-7a(5H)-yl) methoxy)-11-methyl-8,9-dihydro-11H-7-oxa-1,3,6,11-tetraazaspiro[cycloocta[de]naphthalene-10,1′-cyclopropan]-1(11a), 2,3a, 3 al(6a), 5-pentaen-5-yl)-5-methyl-4-(trifluoromethyl)aniline

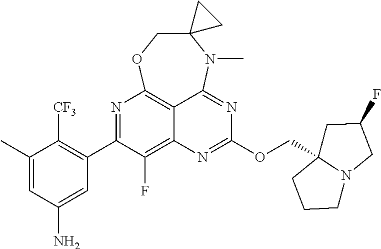 [SMILES: Cc1cc(N)cc(-c2nc3c4c(nc(OC[C@@]56CCCN5C[C@H](F)C6)nc4c2F)N(C)C2(CC2)CO3)c1C(F)(F)F]

Example 53 was prepared by a procedure similar to that described in Example 31 by replacing 3-chloro-5-(4,4,5,5-tetramethyl-1,3,2-dioxaborolan-2-yl)-4-(trifluoromethyl)aniline with 3-methyl-5-(4,4,5,5-tetramethyl-1,3,2-dioxaborolan-2-yl)-4-(trifluoromethyl)aniline in the last step to give the title product (13.7 mg). ¹H NMR (500 MHZ, CD₃OD) & 6.78-6.60 (m, 1H), 6.60-6.34 (m, 1H), 5.41-5.21 (m, 1H), 4.58-4.48 (m, 2H), 4.36-4.18 (m, 2H), 3.36 (s, 3H), 3.27-3.23 (m, 2H), 3.09-3.00 (m, 1H), 2.68-1.48 (m, 11H), 1.10-0.42 (m, 4H). MS (ESI, m/e) [M+H]⁺ 591.2.

Example 54: 3-(11-ethyl-4-fluoro-2-(((2R,7aS)-2-fluorotetrahydro-1H-pyrrolizin-7a(5H)-yl)methoxy)-8,9-dihydro-11H-7-oxa-1,3,6,11-tetraazaspiro[cycloocta[de]naphthalene-10,1′-cyclopropan]-1(11a), 2,3a, 3a¹(6a), 5-pentaen-5-yl)-5-methyl-4-(trifluoromethyl)aniline

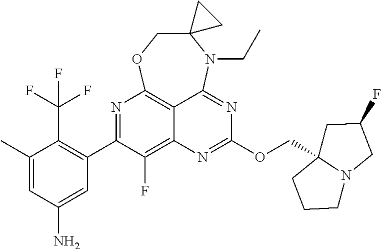 [SMILES: CCN1c2nc(OC[C@@]34CCCN3C[C@H](F)C4)nc3c(F)c(-c4cc(N)cc(C)c4C(F)(F)F)nc(c23)OCC12CC2]

Example 54 was prepared by a procedure similar to that described in Example 31/53 by replacing 2-(1-(methylamino) cyclopropyl) ethan-1-ol and 3-chloro-5-(4,4,5,5-tetramethyl-1,3,2-dioxaborolan-2-yl)-4-(trifluoromethyl)aniline with 2-(1-(ethylamino)cyclopropyl)ethan-1-ol and 3-methyl-5-(4,4,5,5-tetramethyl-1,3,2-dioxaborolan-2-yl)-4-(trifluoromethyl)aniline to give the title product (11 mg). ¹H NMR (500 MHZ, CD₃OD) δ6.72-6.64 (m, 1H), 6.60-6.30 (m, 1H), 5.43-5.25 (m, 1H), 4.60-4.54 (m, 2H), 4.43-4.30 (m, 2H), 4.10-4.04 (m, 1H), 3.92-3.77 (m, 1H), 3.40-3.32 (m, 2H), 3.14-3.04 (m, 1H), 2.69-1.90 (m, 10H), 1.70-1.53 (m, 1H), 1.50-1.40 (m, 3H), 1.20-0.80 (m, 3H), 0.70-0.43 (m, 1H). MS (ESI, m/e) [M+H]⁺ 605.4.

Example 55: 3-chloro-5-(11-ethyl-4-fluoro-2-(((2R,7aS)-2-fluorotetrahydro-1H-pyrrolizin-7a(5H)-yl)methoxy)-8,9-dihydro-11H-7-oxa-1,3,6,11-tetraazaspiro[cycloocta[de]naphthalene-10,1′-cyclopropan]-1(11a), 2,3a, 3 al(6a), 5-pentaen-5-yl)-4-(trifluoromethyl)aniline

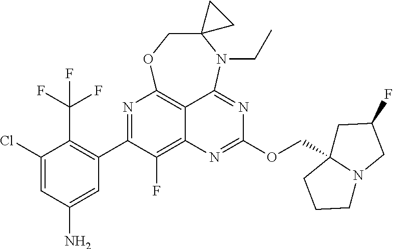 [SMILES: CCN1c2nc(OC[C@@]34CCCN3C[C@H](F)C4)nc3c(F)c(-c4cc(N)cc(Cl)c4C(F)(F)F)nc(c23)OCC12CC2]

Example 55 was prepared by a procedure similar to that described in Example 54 by replacing 3-methyl-5-(4,4,5,5-tetramethyl-1,3,2-dioxaborolan-2-yl)-4-(trifluoromethyl)aniline with 3-chloro-5-(4,4,5,5-tetramethyl-1,3,2-dioxaborolan-2-yl)-4-(trifluoromethyl)aniline to give the title product (16 mg). ¹H NMR (500 MHZ, DMSO-d₆) δ 6.90-6.80 (m, 1H), 6.65-6.28 (m, 3H), 5.38-5.20 (m, 1H), 4.62-4.50 (m, 2H), 4.13-3.58 (m, 4H), 3.36-3.30 (m, 2H), 3.10-2.89 (m, 3H), 2.85-2.78 (m, 1H), 2.16-1.87 (m, 3H), 1.84-1.72 (m, 3H), 1.57-1.46 (m, 1H), 1.34-1.28 (m, 3H), 1.07-0.98 (m, 1H), 0.87-0.74 (m, 2H). MS (ESI, m/e) [M+H]⁺ 625.4.

Example 56: 3-(11-ethyl-4-fluoro-2-(((2R,7aS)-2-fluorotetrahydro-1H-pyrrolizin-7a(5H)-yl)methoxy)-8,9-dihydro-11H-7-oxa-1,3,6,11-tetraazaspiro[cycloocta[de]naphthalene-10,3′-oxetan]-1(11a), 2,3a, 3a¹(6a),5-pentaen-5-yl)-5-methyl-4-(trifluoromethyl)aniline

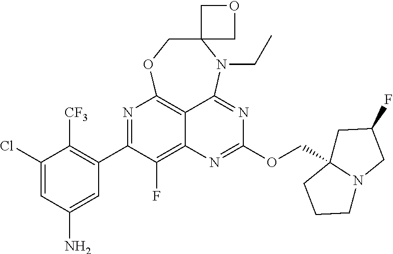 [SMILES: CCN1c2nc(OC[C@@]34CCCN3C[C@H](F)C4)nc3c(F)c(-c4cc(N)cc(Cl)c4C(F)(F)F)nc(c23)OCC12COC2]

Example 56 was prepared by a procedure similar to that described in Example 35 by replacing 3-chloro-5-(4,4,5,5-tetramethyl-1,3,2-dioxaborolan-2-yl)-4-(trifluoromethyl)aniline with 3-methyl-5-(4,4,5,5-tetramethyl-1,3,2-dioxaborolan-2-yl)-4-(trifluoromethyl)aniline to give the title product (18.5 mg). ¹H NMR (500 MHZ, CD₃OD) & 6.67 (s, 1H), 6.51-6.39 (m, 1H), 5.48-5.27 (m, 1H), 4.69-4.48 (m, 4H), 4.46-4.29 (m, 2H), 3.80-3.58 (m, 2H), 3.51-3.32 (m, 5H), 3.18-3.08 (m, 1H), 2.47-1.89 (m, 9H), 1.54-1.45 (m, 3H). MS (ESI, m/e) [M+H]⁺ 621.4.

Example 57: 3-((7aR,11aS)-12-ethyl-4-fluoro-2-(((2R,7aS)-2-fluorotetrahydro-1H-pyrrolizin-7a(5H)-yl)methoxy)-7a, 8,10,11,11a, 12-hexahydro-7,9-dioxa-1,3,6,12-tetraazapleiaden-5-yl)-5-methyl-4-(trifluoromethyl)aniline

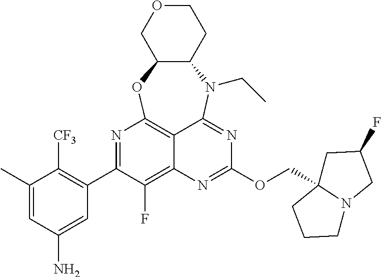 [SMILES: CCN1c2nc(OC[C@@]34CCCN3C[C@H](F)C4)nc3c(F)c(-c4cc(N)cc(C)c4C(F)(F)F)nc(c23)O[C@H]2COCC[C@@H]21]

Step 1: (7aR,11aS)-5-chloro-12-ethyl-4-fluoro-2-(methylthio)-7a, 8,10,11,11a, 12-hexahydro-7,9-dioxa-1,3,6,12-tetraazapleiadene

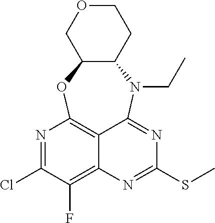 [SMILES: CCN1c2nc(SC)nc3c(F)c(Cl)nc(c23)O[C@H]2COCC[C@@H]21]

To a stirred solution of (7aR,11aS)-5-chloro-4-fluoro-2-(methylthio)-7a, 8,10,11,11a, 12-hexahydro-7,9-dioxa-1,3,6,12-tetraazapleiadene (482 mg, 1.4 mmol) in DMF (15 mL) was added NaH (168 mg, 4.2 mmol, 60%) at 0° C., the resulting mixture was stirred at 0° C. for 15 min. Then to the above mixture was added CH₃CH₂I (655 mg, 4.2 mmol), the reaction mixture was stirred for 6 hrs at 50° C. The reaction was quenched with water, the crude was purified by flash chromatography (PE/EtOAc=5:1 to 1:1) to afford the desired product (466 mg). MS (ESI, m/e) [M+H]⁺ 371.2.

Step 2: (7aR,11aS)-5-chloro-12-ethyl-4-fluoro-2-(methylsulfinyl)-7a, 8,10,11,11a, 12-hexahydro-7,9-dioxa-1,3,6,12-tetraazapleiadene

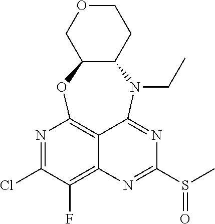 [SMILES: CCN1c2nc(S(C)=O)nc3c(F)c(Cl)nc(c23)O[C@H]2COCC[C@@H]21]

To a stirred solution of (7aR,11aS)-5-chloro-12-ethyl-4-fluoro-2-(methylthio)-7a, 8,10,11,11a, 12-hexahydro-7,9-dioxa-1,3,6,12-tetraazapleiadene (466 mg, 1.26 mmol) in DCM (10 mL) was added m-CPBA (256 mg, 1.26 mmol, 85%) at 0° C., the resulting mixture was stirred at 0° C. for 15 min. The reaction was quenched with aq. Na₂S₂O₃, then extracted with DCM. The organic layer was washed with Sat, aq NaHCO₃ and brine, dried over anhydrous Na₂SO₄, concentrated in vacuo. The residue was directly used in the next step without further purification.

Step 3: (7aR,11aS)-5-chloro-12-ethyl-4-fluoro-2-(((2R,7aS)-2-fluorotetrahydro-1H-pyrrolizin-7a(5H)-yl)methoxy)-7a, 8,10,11,11a, 12-hexahydro-7,9-dioxa-1,3,6,12-tetraazapleiadene

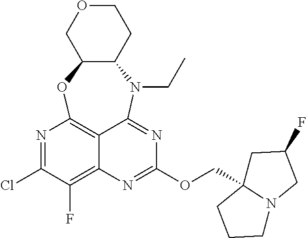 [SMILES: CCN1c2nc(OC[C@@]34CCCN3C[C@H](F)C4)nc3c(F)c(Cl)nc(c23)O[C@H]2COCC[C@@H]21]

To a stirred solution of (7aR,11aS)-5-chloro-12-ethyl-4-fluoro-2-(methylsulfinyl)-7a, 8,10,11,11a, 12-hexahydro-7,9-dioxa-1,3,6,12-tetraazapleiadene (1.26 mmol) and ((2R,7aS)-2-fluorotetrahydro-1H-pyrrolizin-7a(5H)-yl)methanol (400 mg, 2.52 mmol) in THF (15 mL) was added LiHMDS (2.5 mL, 2.52 mmol, 1M in THF) dropwise at 0° C., the resulting mixture was stirred at 0° C. for 20 min. The reaction was quenched with Sat, aq NH₄Cl, then extracted with DCM. The organic layer was washed with brine, dried over anhydrous Na₂SO₄, and concentrated in vacuo. The residue was purified by flash chromatography (DCM/MeOH=100:1 to 30:1) to afford the product (270 mg). MS (ESI, m/e) [M−H]: 482.4.

Step 4: 3-((7aR,11aS)-12-ethyl-4-fluoro-2-(((2R,7aS)-2-fluorotetrahydro-1H-pyrrolizin-7a(5H)-yl)methoxy)-7a, 8,10,11,11a, 12-hexahydro-7,9-dioxa-1,3,6,12-tetraazapleiaden-5-yl)-5-methyl-4-(trifluoromethyl)aniline

To a mixture of (7aR,11aS)-5-chloro-12-ethyl-4-fluoro-2-(((2R,7aS)-2-fluorotetrahydro-1H-pyrrolizin-7a(5H)-yl)methoxy)-7a, 8,10,11,11a, 12-hexahydro-7,9-dioxa-1,3,6,12-tetraazapleiadene (80 mg, 0.17 mmol), 3-methyl-5-(4,4,5,5-tetramethyl-1,3,2-dioxaborolan-2-yl)-4-(trifluoromethyl)aniline (75 mg, 0.25 mmol), Pd(dtbpf)Cl₂ (22 mg, 0.03 mmol) and NaHCO₃ (29 mg, 0.34 mmol) was added dioxane (5.0 mL) and H₂O (1.0 mL), and the reaction mixture was stirred at 90° C. for 1 h. The reaction mixture was concentrated and purified by flash chromatography (DCM/MeOH=100:1 to 5:1), then prep-HPLC to give the product (36.2 mg). ¹H NMR (500 MHZ, CD₃OD) δ6.68 (s, 1H), 6.55-6.21 (m, 1H), 5.48-5.17 (m, 1H), 4.49-4.17 (m, 4H), 4.13-3.81 (m, 4H), 3.59-3.41 (m, 2H), 3.11-2.97 (m, 1H), 2.62-1.68 (m, 11H), 1.61-1.34 (m, 3H). MS (ESI, m/e) [M+H]*621.4.

Example 58: 3-chloro-5-((7aR,11aS)-12-ethyl-4-fluoro-2-(((2R,7aS)-2-fluorotetrahydro-1H-pyrrolizin-7a(5H)-yl)methoxy)-7a, 8,10,11,11a, 12-hexahydro-7,9-dioxa-1,3,6,12-tetraazaplciaden-5-yl)-4-(trifluoromethyl)aniline

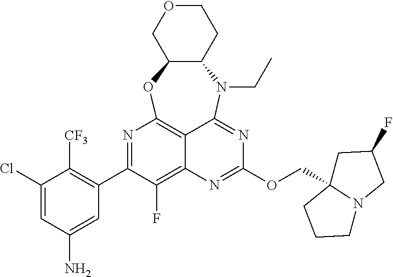 [SMILES: CCN1c2nc(OC[C@@]34CCCN3C[C@H](F)C4)nc3c(F)c(-c4cc(N)cc(Cl)c4C(F)(F)F)nc(c23)O[C@H]2COCC[C@@H]21]

To a mixture of (7aR,11aS)-5-chloro-12-ethyl-4-fluoro-2-(((2R,7aS)-2-fluorotetrahydro-1H-pyrrolizin-7a(5H)-yl)methoxy)-7a, 8,10,11,11a, 12-hexahydro-7,9-dioxa-1,3,6,12-tetraazapleiadene (126 mg, 0.26 mmol), 3-chloro-5-(4,4,5,5-tetramethyl-1,3,2-dioxaborolan-2-yl)-4-(trifluoromethyl)aniline (100 mg, 0.31 mmol), Pd(dtbpf)Cl₂ (33 mg, 0.05 mmol) and NaHCO₃ (44 mg, 0.52 mmol) was added dioxane (10 mL) and H₂O(2.0 mL), and the resulting mixture was stirred at 90° C. for 1 h. The reaction mixture was concentrated and purified by flash chromatography (DCM/MeOH=100:1 to 5:1), then prep-HPLC to give the product (48.5 mg). ¹H NMR (500 MHZ, CD₃OD) δ6.88 (s, 1H), 6.62-6.29 (m, 1H), 5.47-5.22 (m, 1H), 4.45-4.20 (m, 4H), 4.11-3.96 (m, 3H), 3.95-3.82 (m, 1H), 3.55-3.32 (m, 5H), 3.15-3.03 (m, 1H), 2.59-1.73 (m, 8H), 1.51-1.34 (m, 3H). MS (ESI, m e) [M+H]⁺ 641.4.

Example 59: 3-((7aR,11aS)-4-fluoro-2-(((2R,7aS)-2-fluorotetrahydro-1/-pyrrolizin-7a(5H)-yl)methoxy)-12-methyl-7a, 8,10,11,11a, 12-hexahydro-7,9-dioxa-1,3,6,12-tetraazapleiaden-5-yl)-5-methyl-4-(trifluoromethyl)aniline

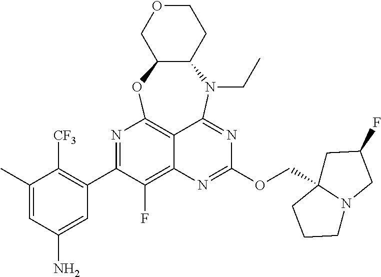 [SMILES: CCN1c2nc(OC[C@@]34CCCN3C[C@H](F)C4)nc3c(F)c(-c4cc(N)cc(C)c4C(F)(F)F)nc(c23)O[C@H]2COCC[C@@H]21]

Step 1: 4,5,7-trichloro-8-fluoro-2-(methylthio)pyrido[4,3-d]pyrimidine

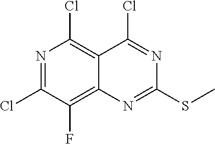 [SMILES: CSc1nc(Cl)c2c(Cl)nc(Cl)c(F)c2n1]

To a solution of 5,7-dichloro-8-fluoro-2-(methylthio)pyrido[4,3-d]pyrimidin-4-ol (1,12 g, 4.0 mmol) in MeCN (30 mL) was added DIEA (774 mg, 6.0 mmol) and POCl₃(797 mg, 5.2 mmol), and the resulting mixture was stirred for 2 hrs at 80° C. The reaction mixture was concentrated under reduced pressure and directly used in the next step without further purification.

Step 2: (3R,4S)-4-((5,7-dichloro-8-fluoro-2-(methylthio)pyrido[4,3-d]pyrimidin-4-yl)(methyl)amino)tetrahydro-2H-pyran-3-ol

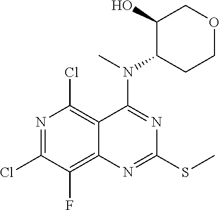 [SMILES: CSc1nc(N(C)[C@H]2CCOC[C@@H]2O)c2c(Cl)nc(Cl)c(F)c2n1]

To a stirred solution of 4,5,7-trichloro-8-fluoro-2-(methylthio)pyrido[4,3-d]pyrimidine (4.0 mmol crude) in DCM (30 mL) was added DIEA (1.3 g, 10 mmol) and (3R,4S)-4-(methylamino)tetrahydro-2H-pyran-3-ol hydrochloride (704 mg, 4.2 mmol) at 0° C., the resulting mixture was stirred for 30 min at 0° C. The reaction mixture was concentrated and purified by flash chromatography (PE/EtOAc=4:1 to 1:2) to afford the desired product. MS (ESI, m/e) [M+H]⁺ 393.2.

Step 3: (7aR,11aS)-5-chloro-4-fluoro-12-methyl-2-(methylthio)-7a, 8,10,11,11a, 12-hexahydro-7,9-dioxa-1,3,6,12-tetraazapleiadene

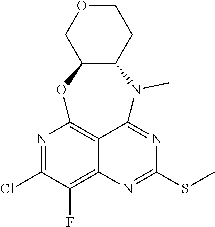 [SMILES: CSc1nc2c3c(nc(Cl)c(F)c3n1)O[C@H]1COCC[C@@H]1N2C]

To a stirred solution of (3R,4S)-4-((5,7-dichloro-8-fluoro-2-(methylthio)pyrido[4,3-d]pyrimidin-4-yl)(methyl)amino)tetrahydro-2H-pyran-3-ol (1.55 g, 3.9 mmol) in THF (30 mL) was added NaH (236 mg, 5.9 mmol, 60%) at 0° C., the resulting mixture was stirred at room temperature for 1 h. The reaction was quenched with H₂O, then extracted with EtOAc. The organic layer was washed with brine, dried over anhydrous Na₂SO₄, concentrated in vacuo. The residue was purified by flash chromatography (PE/EtOAc=2:1 to 1:2) to afford the desired product. MS (ESI, m/e) [M+H]⁺ 357.2.

Step 4: (7aR,11aS)-5-chloro-4-fluoro-12-methyl-2-(methylsulfonyl)-7a, 8,10,11,11a, 12-hexahydro-7,9-dioxa-1,3,6,12-tetraazapleiadene

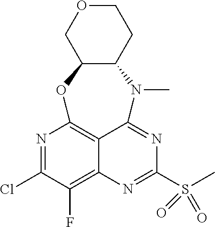 [SMILES: CN1c2nc(S(C)(=O)=O)nc3c(F)c(Cl)nc(c23)O[C@H]2COCC[C@@H]21]

To a stirred solution of (7aR,11aS)-5-chloro-4-fluoro-12-methyl-2-(methylthio)-7a, 8,10,11,11a, 12-hexahydro-7,9-dioxa-1,3,6,12-tetraazapleiadene (545 mg, 1.5 mmol) in THF (16 mL) and H₂O (4.0 mL) was added RuCl₃ (31 mg, 0.15 mmol). Then, to the above mixture was added NaIO₄ (963 mg, 4.5 mmol) in portions at 0° C. The resulting mixture was stirred for 1 h at room temperature. The resulting mixture was diluted with water (30 mL). The resulting mixture was extracted with EtOAc (3×20 mL). The combined organic layers were washed with brine (30 mL), dried over anhydrous Na₂SO₄. After filtration, the filtrate was concentrated under reduced pressure. The residue was purified by silica gel column chromatography, eluted with DCM EtOAc (1:1) to give the title product. MS (ESI, m/e) (M+H)⁺389.1.

Step 5: (7aR,11aS)-5-chloro-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiadene

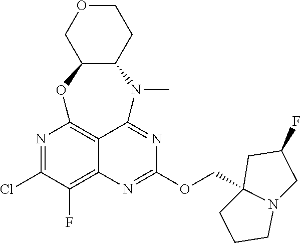 [SMILES: CN1c2nc(OC[C@@]34CCCN3C[C@H](F)C4)nc3c(F)c(Cl)nc(c23)O[C@H]2COCC[C@@H]21]

To a stirred solution of ((2R,7aS)-2-fluorotetrahydro-1H-pyrrolizin-7a(5H)-yl)methanol (95 mg, 0.6 mmol) in THF (1.5 mL) was added LiHMDS (0.6 mL, 1.0M in THF) at 0° C., the resulting mixture was stirred for 10 min at this temperature. Then the above mixture was added to the mixture of (7aR,11aS)-5-chloro-4-fluoro-12-methyl-2-(methylsulfonyl)-7a, 8,10,11,11a, 12-hexahydro-7,9-dioxa-1,3,6,12-tetraazapleiadene (122 mg, 0.3 mmol) and 4A MS (150 mg, the 4A MS was previously roasted for 4 h in a Muffle oven at 400 degrees) in THF (3.0 mL) at 0° C., the resulting mixture was stirred for another 5 min at 0° C. The reaction mixture was diluted with water (30 mL), and extracted with DCM (3×20 mL). The combined organic layers were washed with brine (30 mL), dried over anhydrous Na₂SO₄. After filtration, the filtrate was concentrated under reduced pressure. The residue was purified by silica gel column chromatography, eluted with DCM MeOH (30:1) to give the title product. MS (ESI, m/e) (M+H)⁺468.3.

Step 6: 3-((7aR,11aS)-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiaden-5-yl)-5-methyl-4-(trifluoromethyl)aniline

To a mixture of (7aR,11aS)-5-chloro-4-fluoro-2-(((2R,7aS)-2-fluorotetrahydro-1H-pyrrolizin-7a(5H)-yl)methoxy)-12-methyl-7a, 8,10,11,11a, 12-hexahydro-7,9-dioxa-1,3,6,12-tetraazapleiadene (469 mg, 1.25 mmol), 3-methyl-5-(4,4,5,5-tetramethyl-1,3,2-dioxaborolan-2-yl)-4-(trifluoromethyl)aniline (566 mg, 1.88 mmol), Pd(dtbpf)Cl₂ (163 mg, 0.25 mmol) and NaHCO₃(315 mg, 3.75 mmol) was added dioxane (15 mL) and H₂O (3.0 mL). The reaction mixture was stirred at 90° C. for 1 h. The reaction mixture was concentrated and purified by flash chromatography (DCM MeOH=100:1 to 10:1), then prep-HPLC to give the product. ¹H NMR (500 MHZ, CD₃OD) à 6.68 (s, 1H), 6.57-6.25 (m, 1H), 5.50-5.26 (m, 1H), 4.50-4.32 (m, 3H), 4.31-4.18 (m, 1H), 4.13-3.92 (m, 2H), 3.61-3.09 (m, 9H), 2.60-2.49 (m, 1H), 2.48-2,26 (m, 5H), 2.26-2.17 (m, 1H), 2.16-2.04 (m, 2H), 2.03-1.90 (m, 1H), 1.80-1.62 (m, 1H). MS (ESI, m/e) [M+H]⁺ 607.4.

Example 60: 3-chloro-5-((7aR,11aS)-4-fluoro-12-methyl-2-((7-methyloctahydropyrido[2,1-c][1,4]oxazin-7-yl)methoxy)-7a, 8,10,11,11a, 12-hexahydro-7,9-dioxa-1,3,6,12-tetraazapleiaden-5-yl)-4-(trifluoromethyl)aniline

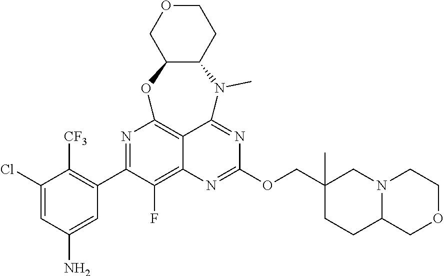 [SMILES: CN1c2nc(OCC3(C)CCC4COCCN4C3)nc3c(F)c(-c4cc(N)cc(Cl)c4C(F)(F)F)nc(c23)O[C@H]2COCC[C@@H]21]

Step 1: (7aR,11aS)-5-chloro-4-fluoro-12-methyl-2-((7-methyloctahydropyrido[2,1-c][1,4]oxazin-7-yl)methoxy)-7a, 8,10,11,11a, 12-hexahydro-7,9-dioxa-1,3,6,12-tetraazapleiadene

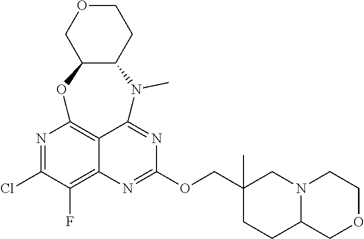 [SMILES: CN1c2nc(OCC3(C)CCC4COCCN4C3)nc3c(F)c(Cl)nc(c23)O[C@H]2COCC[C@@H]21]

To a stirred mixture of (7aR,11aS)-5-chloro-4-fluoro-12-methyl-2-(methylsulfonyl)-7a, 8,10,11,11a, 12-hexahydro-7,9-dioxa-1,3,6,12-tetraazapleiadene (200 mg, 0.5 mmol), (7-methyloctahydropyrido[2,1-c][1,4]oxazin-7-yl)methanol (185 mg, 1.0 mmol) and 4A MS (500 mg, the 4A MS was previously roasted for 4 h in a Muffle oven at 400 degrees) in THF (4.0 mL) was added LiHMDS (1.0 mL, 1.0M in THF) at 0° C., the resulting mixture was stirred for 10 min at 0° C. The reaction mixture was diluted with water (30 mL), and extracted with DCM (3×20 mL). The combined organic layers were washed with brine (30 mL), dried over anhydrous Na₂SO₄. After filtration, the filtrate was concentrated under reduced pressure. The residue was purified by silica gel column chromatography, eluted with DCM/MeOH (30:1) to give the title product. MS (ESI, m e) (M+H)⁺494.4.

Step 2: 3-chloro-5-((7aR,11aS)-4-fluoro-12-methyl-2-((7-methyloctahydropyrido[2,1-c][1,4]oxazin-7-yl)methoxy)-7a, 8,10,11,11a, 12-hexahydro-7,9-dioxa-1,3,6,12-tetraazapleiaden-5-yl)-4-(trifluoromethyl)aniline

To a mixture of (7aR,11aS)-5-chloro-4-fluoro-12-methyl-2-((7-methyloctahydropyrido[2,1-c][1,4]oxazin-7-yl)methoxy)-7a, 8,10,11,11a, 12-hexahydro-7,9-dioxa-1,3,6,12-tetraazapleiadene (100 mg, 0.20 mmol), 3-chloro-5-(4,4,5,5-tetramethyl-1,3,2-dioxaborolan-2-yl)-4-(trifluoromethyl)aniline (77 mg, 0.24 mmol), Pd(dtbpf)Cl₂ (26 mg, 0.04 mmol) and NaHCO₃(34 mg, 0.40 mmol) was added dioxane (7.5 mL) and H₂O (1.5 mL). The reaction mixture was stirred at 90° C. for 1 h. The reaction mixture was concentrated and purified by flash chromatography (DCM/MeOH=100:1 to 10:1), then prep-HPLC to give the product. ¹H NMR (500 MHZ, CD₃OD) δ6.88 (s, 1H), 6.65-6.29 (m, 1H), 4.67-4.46 (m, 2H), 4.44-4.32 (m, 1H), 4.32-4.20 (m, 1H), 4.15-3.96 (m, 2H), 3.88-3.75 (m, 1H), 3.74-3.59 (m, 2H), 3.56-3.43 (m, 2H), 3.40-3.33 (m, 3H), 3.30-3.20 (m, 1H), 2.98-2.83 (m, 1H), 2.70-2.49 (m, 1H), 2.42-2,24 (m, 1H), 2.16-2.04 (m, 1H), 2.04-1.97 (m, 1H), 1.96-1.85 (m, 1H), 1.82-1.64 (m, 1H), 1.49-1.28 (m, 3H), 1.05 (s, 3H). MS (ESI, m/e) (M+H)⁺653.4.

Example 61: 3-((7aR,11aR)-4-fluoro-2-(((2R,7aS)-2-fluorotetrahydro-1H-pyrrolizin-7a(5H)-yl) methoxy)-12-methyl-7a, 8,11a, 12-tetrahydro-9H, 11H-7,10-dioxa-1,3,6,12-tetraazapleiaden-5-yl)-5-methyl-4-(trifluoromethyl)aniline

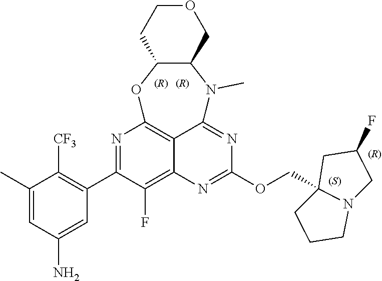

Example 61 was prepared by a procedure similar to that described in Example 51 by replacing 3-chloro-5-(4,4,5,5-tetramethyl-1,3,2-dioxaborolan-2-yl)-4-(trifluoromethyl)aniline with 3-methyl-5-(4,4,5,5-tetramethyl-1,3,2-dioxaborolan-2-yl)-4-(trifluoromethyl)aniline to give the title product (33 mg). ¹H NMR (500 MHZ, CD₃OD) δ 6.75-6.60 (m, 1H), 6.52-6.30 (m, 1H), 5.47-5.25 (m, 1H), 4.62-4.26 (m, 4H), 4.10-3.86 (m, 2H), 3.55-3.34 (m, 4H), 3.27-3.19 (m, 1H), 3.16-3.05 (m, 1H), 2.47-1.84 (m, 11H). MS (ESI, m/e) [M+H]⁺ 607.4.

Example 62: 3-chloro-5-((7aR,11aS)-4-fluoro-12-methyl-2-(((S)-2-methylenetetrahydro-1H-pyrrolizin-7a(5H)-yl) methoxy)-7a, 8,10,11,11a, 12-hexahydro-7,9-dioxa-1,3,6,12-tetraazapleiaden-5-yl)-4-(trifluoromethyl)aniline

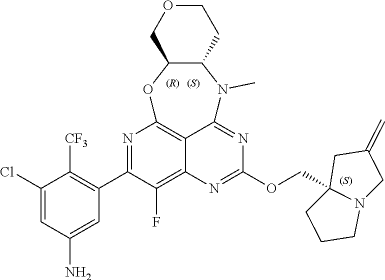

Example 62 was prepared by a procedure similar to that described in Example 49 by replacing ((2R,7aS)-2-fluorotetrahydro-1H-pyrrolizin-7a(5H)-yl)methanol with (S)-(2-methylenetetrahydro-1H-pyrrolizin-7a(5H)-yl) methanol to give the title product (25 mg). ¹H NMR (500 MHz, CD₃OD) δ 6.88 (s, 1H), 6.62-6.32 (m, 1H), 5.09 (s, 2H), 4.53-4.32 (m, 3H), 4.31-4.19 (m, 1H), 4.13-3.90 (m, 3H), 3.60-3.42 (m, 3H), 3.38-3.33 (m, 4H), 2.97-2.79 (m, 2H), 2.66-2.46 (m, 2H), 2.30-2.17 (m, 1H), 2.13-1.88 (m, 3H), 1.78-1.58 (m, 1H). MS (ESI, m/e) [M+H]⁺ 621.4.

Example 63: 3-chloro-5-((7aR,11aS)-2-((2,4-dimethylmorpholin-2-yl)methoxy)-4-fluoro-12-methyl-7a, 8,10,11,11a, 12-hexahydro-7,9-dioxa-1,3,6,12-tetraazapleiaden-5-yl)-4-(trifluoromethyl)aniline

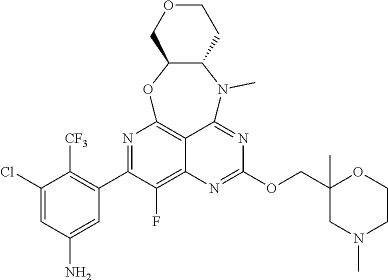 [SMILES: CN1CCOC(C)(COc2nc3c4c(nc(-c5cc(N)cc(Cl)c5C(F)(F)F)c(F)c4n2)O[C@H]2COCC[C@@H]2N3C)C1]

Example 63 was prepared by a procedure similar to that described in Example 49 by replacing ((2R,7aS)-2-fluorotetrahydro-1H-pyrrolizin-7a(5H)-yl)methanol with (2,4-dimethylmorpholin-2-yl)methanol to give the title product (7 mg). ¹H NMR (500 MHZ, DMSO-d₆) δ 6.86 (s, 1H), 6.59-6.25 (m, 3H), 4.72-4.54 (m, 1H), 4.44 (s, 1H), 4.30-4.20 (m, 1H), 4.15-3.86 (m, 3H), 3.77-3.59 (m, 2H), 3.49-3.34 (m, 2H), 3.32 (s, 3H), 2.47-2.43 (m, 2H), 2.33 (s, 1H), 2,25-2.08 (m, 5H), 1.60 (s, 1H), 1.25 (s, 3H). MS (ESI, m/e) [M+H]⁺ 613.4.

Example 64: 3-chloro-5-((7aR,11aS)-4-fluoro-12-methyl-2-(((4aS,7aR)-1-methyloctahydro-4aH-cyclopenta[b]pyridin-4a-yl) methoxy)-7a, 8,10,11,11a, 12-hexahydro-7,9-dioxa-1,3,6,12-tetraazapleiaden-5-yl)-4-(trifluoromethyl)aniline

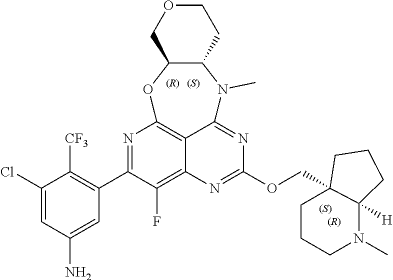

Example 64 was prepared by a procedure similar to that described in Example 49 by replacing ((2R,7aS)-2-fluorotetrahydro-1H-pyrrolizin-7a(5H)-yl)methanol with ((4aS,7aR)-1-methyloctahydro-4aH-cyclopenta[b]pyridin-4a-yl)methanol to give the title product (45 mg). ¹H NMR (500 MHz, CD₃OD) δ 6.88 (s, 1H), 6.61-6.37 (m, 1H), 4.53-4.33 (m, 3H), 4.32-4.20 (m, 1H), 4.12-3.94 (m, 2H), 3.55-3.45 (m, 2H), 3.36 (s, 3H), 3.21-3.04 (m, 1H), 2.83 (s, 1H), 2.69-2.57 (m, 1H), 2.57-2.38 (m, 4H), 2.05-1.84 (m, 3H), 1.83-1.69 (m, 7H), 1.66-1.57 (m, 1H). MS (ESI, m/e) [M+H]⁺ 637.4.

Example 65: 3-((7aR,11aS)-4-fluoro-12-methyl-2-(((4aS,7aR)-1-methyloctahydro-4aH-cyclopenta[b]pyridin-4a-yl)methoxy)-7a, 8,10,11,11a, 12-hexahydro-7,9-dioxa-1,3,6,12-tetraazapleiaden-5-yl)-5-methyl-4-(trifluoromethyl)aniline

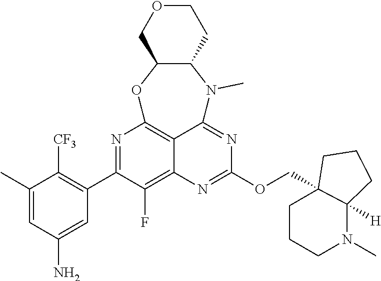

Example 65 was prepared by a procedure similar to that described in Example 64 by replacing 3-chloro-5-(4,4,5,5-tetramethyl-1,3,2-dioxaborolan-2-yl)-4-(trifluoromethyl)aniline with 3-methyl-5-(4,4,5,5-tetramethyl-1,3,2-dioxaborolan-2-yl)-4-(trifluoromethyl)aniline to give the title product (43 mg). ¹H NMR (500 MHZ, CD₃OD) δ6.69 (s, 1H), 6.53-6.28 (m, 1H), 4.56-4.48 (m, 1H), 4.46-4.32 (m, 2H), 4.27 (s, 1H), 4.12-3.95 (m, 2H), 3.69 (s, 1H), 3.51-3.44 (m, 2H), 3.36 (s, 3H), 3.28-3.20 (m, 1H), 3.19-3.09 (m, 1H), 2.83 (s, 3H), 2.59-2.49 (m, 1H), 2.41 (s, 3H), 2,25-2.11 (m, 2H), 2.05-1.94 (m, 2H), 1.91-1.67 (m, 7H). MS (ESI, m/e) [M+H]⁺ 617.4.

Example 66: 3-chloro-2-fluoro-5-((7aR,11aS)-4-fluoro-2-(((2R,7aS)-2-fluorotetrahydro-1H-pyrrolizin-7a(5H)-yl) methoxy)-12-methyl-7a, 8,10,11,11a, 12-hexahydro-7,9-dioxa-1,3,6,12-tetraazapleiaden-5-yl)-4-(trifluoromethyl)aniline

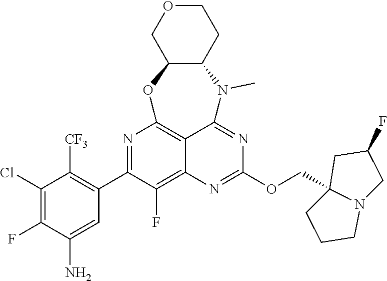 [SMILES: CN1c2nc(OC[C@@]34CCCN3C[C@H](F)C4)nc3c(F)c(-c4cc(N)c(F)c(Cl)c4C(F)(F)F)nc(c23)O[C@H]2COCC[C@@H]21]

Example 66 was prepared by a procedure similar to that described in Example 59 by replacing 3-chloro-5-(4,4,5,5-tetramethyl-1,3,2-dioxaborolan-2-yl)-4-(trifluoromethyl)aniline with 3-chloro-2-fluoro-5-(4,4,5,5-tetramethyl-1,3,2-dioxaborolan-2-yl)-4-(trifluoromethyl)aniline to give the title product (72 mg). ¹H NMR (500 MHZ, CD₃OD) δ 6.85-6.52 (m, 1H), 5.49-5.28 (m, 1H), 4.51-4.22 (m, 4H), 4.17-3.92 (m, 2H), 3.53-3.41 (m, 5H), 3.34 (s, 3H), 3.21-3.12 (m, 1H), 2.59-2.18 (m, 4H), 2.16-1.91 (m, 3H), 1.72 (s, 1H). MS (ESI, m/e) [M+H]⁺ 645.4.

Example 67: 2-fluoro-5-((7aR,11aS)-4-fluoro-2-(((2R,7aS)-2-fluorotetrahydro-1H-pyrrolizin-7a(5H)-yl) methoxy)-12-methyl-7a, 8,10,11,11a, 12-hexahydro-7,9-dioxa-1,3,6,12-tetraazapleiaden-5-yl)-3-methyl-4-(trifluoromethyl)aniline

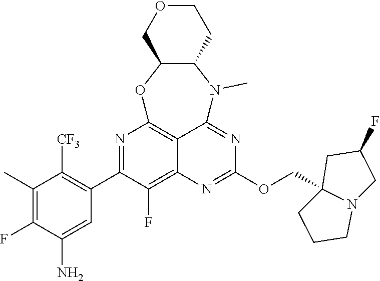 [SMILES: Cc1c(F)c(N)cc(-c2nc3c4c(nc(OC[C@@]56CCCN5C[C@H](F)C6)nc4c2F)N(C)[C@H]2CCOC[C@@H]2O3)c1C(F)(F)F]

Example 67 was prepared by a procedure similar to that described in Example 59 by replacing 3-chloro-5-(4,4,5,5-tetramethyl-1,3,2-dioxaborolan-2-yl)-4-(trifluoromethyl)aniline with 2-fluoro-3-methyl-5-(4,4,5,5-tetramethyl-1,3,2-dioxaborolan-2-yl)-4-(trifluoromethyl)aniline to give the title product (35 mg). ¹H NMR (500 MHZ, CD₃OD) δ6.68-6.45 (m, 1H), 5.49-5.28 (m, 1H), 4.45-4.21 (m, 4H), 4.11-3.94 (m, 2H), 3.58-3.38 (m, 5H), 3.36 (s, 3H), 3.21-3.06 (m, 1H), 2.56-1.88 (m, 10H), 1.78-1.66 (m, 1H). MS (ESI, m/e) [M+H]⁺ 625.4.

Example 68 Isomer 1 and Isomer 2:3-chloro-5-((8aR,11aR)-4-fluoro-2-(((2R,7aS)-2-fluorotetrahydro-1H-pyrrolizin-7a(5H)-yl)methoxy)-12-methyl-8a, 9,11a, 12-tetrahydro-8H, 11H-7,10-dioxa-1,3,6,12-tetraazacyclopenta[5,6]cycloocta[1,2,3-de]naphthalen-5-yl)-4-(trifluoromethyl)aniline, and 3-chloro-5-((8aS,11aS)-4-fluoro-2-(((2R,7aS)-2-fluorotetrahydro-1H-pyrrolizin-7a(5H)-yl)methoxy)-12-methyl-8a, 9,11a, 12-tetrahydro-8H, 11H-7,10-dioxa-1,3,6,12-tetraazacyclopenta[5,6]cycloocta[1,2,3-de]naphthalen-5-yl)-4-(trifluoromethyl)aniline

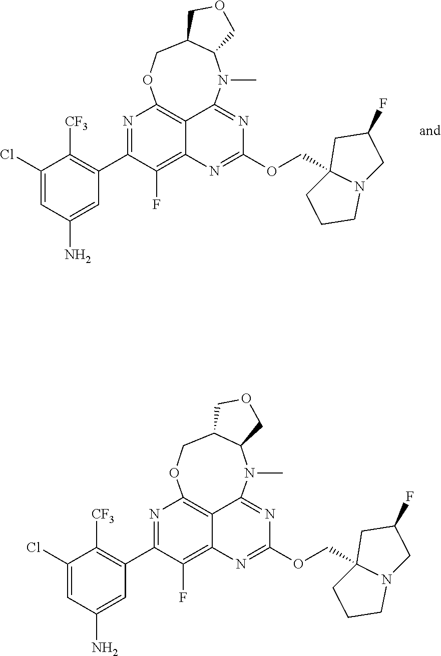 [SMILES: CN1c2nc(OC[C@@]34CCCN3C[C@H](F)C4)nc3c(F)c(-c4cc(N)cc(Cl)c4C(F)(F)F)nc(c23)OC[C@H]2COC[C@@H]21 ; CN1c2nc(OC[C@@]34CCCN3C[C@H](F)C4)nc3c(F)c(-c4cc(N)cc(Cl)c4C(F)(F)F)nc(c23)OC[C@@H]2COC[C@H]21]

Step 1: 7-chloro-8-fluoro-5-((trans-4-(methylamino)tetrahydrofuran-3-yl)methoxy)-2-(methylthio)pyrido[4,3-d]pyrimidin-4-ol

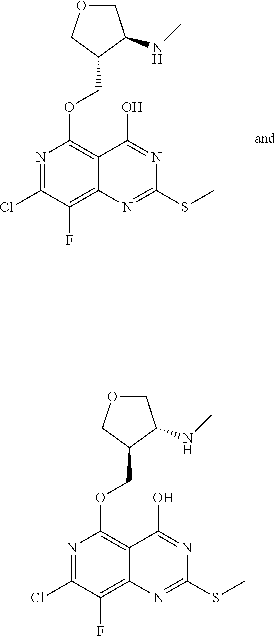 [SMILES: CN[C@@H]1COC[C@H]1COc1nc(Cl)c(F)c2nc(SC)nc(O)c12 ; CN[C@H]1COC[C@@H]1COc1nc(Cl)c(F)c2nc(SC)nc(O)c12]

To a solution of trans-(4-(methylamino)tetrahydrofuran-3-yl)methanol (2 g, 15.2 mmol) and 5,7-dichloro-8-fluoro-2-(methylthio) pyrido[4,3-d]pyrimidin-4-ol (4.2g, 15.2 mmol) in THF (250 mL) was added sodium hydride (3.05g, 76.3 mmol) at room temperature. The resulting mixture was stirred at room temperature for 16 hours. After completion, the reaction mixture was quenched with ice water and concentrated to give the residue. The residue was purified by flash column silica (DCM: MeOH=10:1) to give the title product (1.5 g). MS (ESI, me) [M+H]⁺ 375.

Step 2: (8a, 11a-trans)-5-chloro-4-fluoro-12-methyl-2-(methylthio)-8a, 9,11a, 12-tetrahydro-8H, 11H-7,10-dioxa-1,3,6,12-tetraazacyclopenta[5,6]cycloocta[1,2,3-de]naphthalene

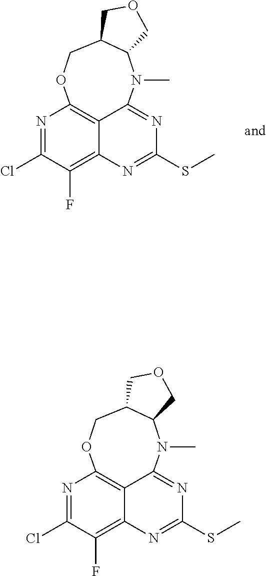 [SMILES: CSc1nc2c3c(nc(Cl)c(F)c3n1)OC[C@H]1COC[C@@H]1N2C ; CSc1nc2c3c(nc(Cl)c(F)c3n1)OC[C@@H]1COC[C@H]1N2C]

To a solution of 7-chloro-8-fluoro-5-((trans-4-(methylamino)tetrahydrofuran-3-yl)methoxy)-2-(methylthio)pyrido[4,3-d]pyrimidin-4-ol (1.5 g, 4 mmol) in 300 mL ACN was added N,N-Diisopropylethylamine (1.5 g, 12 mmol) and Bop-Cl(2.03g, 8 mmol) at room temperature and it was stirred at 70° C. for 16 hrs. After completion, the reaction mixture was quenched with ice water and concentrated to give the residue. The residue was purified by flash column silica (DCM: MeOH=20:1) to give the title product. MS (ESI, m/e) [M+H]⁺ 357.

Step 3: (8a, 11a-trans)-5-chloro-4-fluoro-12-methyl-2-(methylsulfonyl)-8a, 9,11a, 12-tetrahydro-8H, 11H-7,10-dioxa-1,3,6,12-tetraazacyclopenta[5,6]cycloocta[1,2,3-de]naphthalene

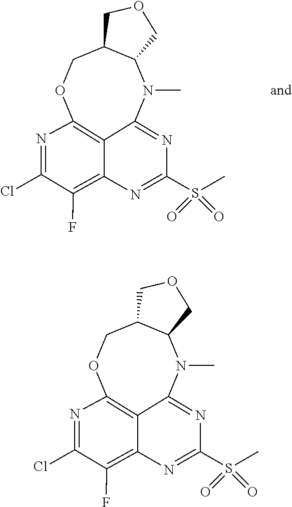 [SMILES: CN1c2nc(S(C)(=O)=O)nc3c(F)c(Cl)nc(c23)OC[C@H]2COC[C@@H]21 ; CN1c2nc(S(C)(=O)=O)nc3c(F)c(Cl)nc(c23)OC[C@@H]2COC[C@H]21]

To a stirred solution of (8a, 11a-trans)-5-chloro-4-fluoro-12-methyl-2-(methylthio)-8a, 9,11a, 12-tetrahydro-8H, 11H-7,10-dioxa-1,3,6,12-tetraazacyclopenta[5,6]cycloocta[1,2,3-de]naphthalene (270 mg, 0.76 mmol) in THF (16 mL) and H₂O (4.0 mL) was added RuCl₃ (23.5 mg, 0.11 mmol). Then, to the above mixture was added NaIO₄ (492 mg, 2,28 mmol) in portions at 0° C. The resulting mixture was stirred for 1 h at room temperature. The resulting mixture was diluted with water (30 mL). The resulting mixture was extracted with EtOAc (3×20 mL). The combined organic layers were washed with brine (30 mL), dried over anhydrous Na₂SO₄. After filtration, the filtrate was concentrated under reduced pressure. The residue was purified by silica gel column chromatography, eluted with DCM EtOAc (1:1) to give the title product. MS (ESI, m/c) [M+H]⁺ 389.1.

Step 4: (8a, 11a-trans)-5-chloro-4-fluoro-2-(((2R,7aS)-2-fluorotetrahydro-1H-pyrrolizin-7a(5H)-yl)methoxy)-12-methyl-8a, 9,11a, 12-tetrahydro-8H, 11H-7,10-dioxa-1,3,6,12-tetraazacyclopenta[5,6]cycloocta| 1,2,3-de|naphthalene

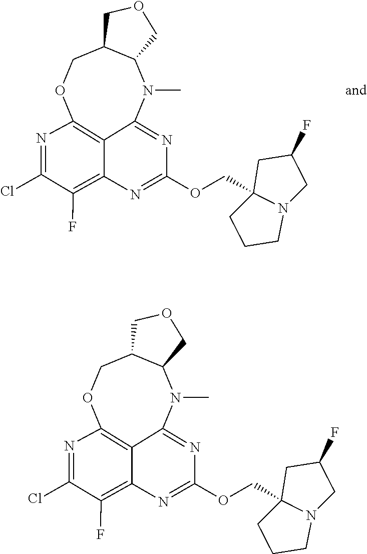 [SMILES: CN1c2nc(OC[C@@]34CCCN3C[C@H](F)C4)nc3c(F)c(Cl)nc(c23)OC[C@H]2COC[C@@H]21 ; CN1c2nc(OC[C@@]34CCCN3C[C@H](F)C4)nc3c(F)c(Cl)nc(c23)OC[C@@H]2COC[C@H]21]

To a stirred solution of ((2R,7aS)-2-fluorotetrahydro-1H-pyrrolizin-7a(5H)-yl)methanol (106 mg, 0.67 mmol) in THF (15 mL) was added LiHMDS (1 mL, 1.0M in THF) at 0° C., the resulting mixture was stirred for 10 min at this temperature. Then the above mixture was added to the mixture of (8a, 11a-trans)-5-chloro-4-fluoro-12-methyl-2-(methylsulfonyl)-8a, 9,11a, 12-tetrahydro-8H, 11H-7,10-dioxa-1,3,6,12-tetraazacyclopenta[5,6]cycloocta[1,2,3-de]naphthalene (130 mg, 0.33 mmol) and 4A MS (150 mg, the 4A MS was previously roasted for 4 h in a Muffle oven at 400 degrees) in THF (3.0 mL) at 0° C., the resulting mixture was stirred for another 5 min at 0° C. The reaction mixture was diluted with water (30 mL), and extracted with DCM (3×20 mL). The combined organic layers were washed with brine (30 mL), dried over anhydrous Na₂SO₄. After filtration, the filtrate was concentrated under reduced pressure. The residue was purified by silica gel column chromatography, eluted with DCM/MeOH (30:1) to give the title product. MS (ESI, m/c) [M+H]: 468.3.

Step 5: 3-chloro-5-((8aR,11aR)-4-fluoro-2-(((2R,7aS)-2-fluorotetrahydro-1H-pyrrolizin-7a(5H)-yl)methoxy)-12-methyl-8a, 9,11a, 12-tetrahydro-8H, 11H-7,10-dioxa-1,3,6,12-tetraazacyclopenta[5,6]cycloocta[1,2,3-de]naphthalen-5-yl)-4-(trifluoromethyl)aniline and 3-chloro-5-((8aS,11aS)-4-fluoro-2-(((2R,7aS)-2-fluorotetrahydro-1H-pyrrolizin-7a(5H)-yl)methoxy)-12-methyl-8a, 9,11a, 12-tetrahydro-8H, 11H-7,10-dioxa-1,3,6,12-tetraazacyclopenta[5,6]cycloocta[1,2,3-de]naphthalen-5-yl)-4-(trifluoromethyl)aniline

To a solution of (8a, 11a-trans)-5-chloro-4-fluoro-2-(((2R,7aS)-2-fluorotetrahydro-1H-pyrrolizin-7a(5H)-yl)methoxy)-12-methyl-8a, 9,11a, 12-tetrahydro-8H, 11H-7,10-dioxa-1,3,6,12-tetraazacyclopenta[5,6]cycloocta[1,2,3-de]naphthalene (130 mg, 0.28 mmol) in dioxane H₂O (20/4 mL) was added 3-chloro-5-(4,4,5,5-tetramethyl-1,3,2-dioxaborolan-2-yl)-4-(trifluoromethyl) aniline (134 mg, 0.42 mmol), NaHCO₃(70 mg, 0.84 mmol) and Pd(dppf)Cl: (41 mg, 0.05 mmol) at room temperature, and the mixture was stirred at 100° C. for 2 hours. The resulting cooled mixture was concentrated and purified by column chromatography (DCM MeOH=10/1) to give a residue which was further purified by Prep-HPLC to give a mixture of Example 68 Isomer 1 and Isomer 2. Isomers in the mixture (88 mg) were further separated from each other using preparative chiral HPLC (Equipment is Prep-HPLC-Gilson GX-281; Column is I-Cellulose-5 4.6*250 mm 5 μm; diluent is ethanol (5.5 mL); injection volume is 1 mL or 1.5 mL each time; Mobile phase A is hexanes; Mobile phase B is ethanol with 0.2% 2M NH₃ in methanol; gradient is Mobile phase A: mobile phase B (50%: 50%, v/v); flow rate is 18 mL/min; temperature is 25° C.; wavelength are UV 220 nm and 240 nm; retention time of Isomer 1 is 6.5 min, and retention time of Isomer 2 is 8.5 min). Analytical chiral HPLC method: Equipment is HPLC-Agilent 1260 Infinity II; Column is ChiralPak IC 4.6*150 mm 5 μM; diluent is ethanol; injection volume is 2 μL; Mobile phase A is hexanes; Mobile phase B is ethanol with 0.1% 2M NH₃ in methanol; gradient is Mobile phase A: mobile phase B (60%: 40%, v/v); flow rate is 1.0 mL/min; temperature is 25° C.; wavelength are UV 214 nm and 254 nm.

Isomer 1: retention time under analytical chiral HPLC method is 4.1 min. ¹H NMR (500 MHz, CD₃OD) δ 6.89 (s, 1H), 6.69-6.36 (m, 1H), 5.45-5.18 (m, 1H), 4.28-3.65 (m, 5H), 3.53-3.38 (m, 1H), 3.32-3.13 (m, 9H), 3.08-2.86 (m, 2H), 2.42-2.08 (m, 3H), 2.06-1.81 (m, 3H). MS (ESI, m/c) [M+H]⁺ 627.4.

Isomer 2: retention time under analytical chiral HPLC method is 6.5 min. ¹H NMR (500 MHz, CD₃OD) δ6.89 (s, 1H), 6.69-6.36 (m, 1H), 5.45-5.18 (m, 1H), 4.40-4.28 (m, 1H), 4.26-4.18 (m, 1H), 4.17-4.03 (m, 2H), 4.00-3.74 (m, 1H), 3.54-3.38 (m, 1H), 3.33-3.15 (m, 9H), 3.08-2.87 (m, 2H), 2.39-2.07 (m, 3H), 2.05-1.81 (m, 3H). [M+H]⁺ 627.4.

### Assays

### KRAS WT and KRAS G12V Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-169) and GST-tagged GDP-loaded KRAS G12V (amino acids 1-169) were expressed in E. coli and purified in house. All protein and reaction solutions were prepared in assay buffer containing 50 mM HEPES pH7.5, 50 mM NaCl, 1 mM MgCl2, 1 mM TCEP, 0.01% BSA, and 0.008% Brij-35. Purified WT KRAS (3 nM final concentration) or KRAS G12V protein (2 nM final concentration) was incubated with a 3-fold serially diluted compound in the assay plate (384 well microplate, black. Corning). Plates are incubated at 24ºC for 1 hr. Following the incubation, biotinylated probe 1 (60 nM final assay concentration) for WT KRAS and biotinylated probe 2 (2.5 nM final assay concentration) for KRAS G12V was added to the assay plate, respectively. After 1 hr incubation at 24° C. Mab Anti-GST-Tb cryptate (Cisbio) and Streptavidin-XL665 (Cisbio) were added and further incubated at 24ºC for another 1 hr. The TR-FRET signals (ex337 nm, em665 nm/620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC5% value of each compound was calculated from fitting the data to the four-parameter logistic model by Dotmatics.

### KRAS WT and KRAS G12D Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-188) and GST-tagged GDP-loaded KRAS G12D (amin acids 1-188) were expressed in E. coli and purified in house. All protein and reaction solutions were prepared in assay buffer containing 50 mM HEPES pH7.5, 50 mM NaCl, 1 mM MgCl2, 1 mM TCEP, 0.01% BSA, and 0.008% Brij-35. Purified WT KRAS (3 nM final concentration) or KRAS G12D protein (0.5 nM final concentration) was incubated with a 3-fold serially diluted compound in the assay plate (384 well microplate, black, Corning). Plates are incubated at 24° C. for 1 hr. Following the incubation, biotinylated probe 1 (60 nM final assay concentration) for WT KRAS and biotinylated probe 2 (4 nM final assay concentration) for KRAS G12D was added to the assay plate, respectively. After 1 hr incubation at 24° C., Mab Anti-GST-Tb cryptate (Cisbio) and Streptavidin-XL665 (Cisbio) were added and further incubated at 24ºC for another 1 hr. The TR-FRET signals (ex337 nm, em665 nm 620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC₅₀ value of each compound was calculated from fitting the data to the four-parameter logistic model by Dotmatics.

### KRAS G12V PERK Assay

SW620 cell line was used in this study. Cells were maintained in RPMI 1640 supplemented with 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the original cells purchased. 40000 cells per well were seeded into a 96-well plate and incubated overnight. Cells were treated with a 10-point dilution series. The final compound concentration is from 0 to 10 μM. After 2 hrs compound treatment, cells were lysed, and the pERK1/2(THR202/TYR204) level in the cell lysates was detected by HTRF kit (Cisbio). In brief, a total of 16 μL of cell lysate from each well of a 96-well plate was transferred to a 384-well white assay plate. Lysate from each well was incubated with 2 μL of Eu3+-cryptate (donor) labeled anti-phospho-ERK1/2 and 2 μL of D)₂ (acceptor) labeled anti-phospho-ERK1/2 antibodies (Cisbio) overnight in dark at room temperature. When donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC₅₀ determination was performed by fitting the curve of percent inhibition versus the log of the inhibitor concentration using Dotmatics.

KRAS G12D pERK Assay

AsPC-1 cell line was used in this study. Cells were maintained in RPMI-1640 supplemented with 10% fetal bovine serum (Thermo Fisher), 50 units/ml, penicillin and streptomycin (Thermo Fisher) and kept at 37° C. in a humidified atmosphere of 5% CO₂ in air. Cells were reinstated from frozen stocks that were laid down within 30 passages from the original cells purchased. 30000 cells per well were seeded into a 96-well plate and incubated overnight. Cells were treated with a 10-point dilution series. The final compound concentration is from 0 to 10 μM. After 2 h compound treatment, cells were lysed, and the pERK1/2(THR202/TYR204) level in the cell lysates was detected by HTRF kit (Cisbio). In brief, a total of 16 μL of cell lysate from each well of a 96-well plate was transferred to a 384-well white assay plate. Lysate from each well was incubated with 2 μL of Eu3+-cryptate (donor) labeled anti-phospho-ERK1/2 and 2 μL of D₂ (acceptor) labeled anti-phospho-ERK1/2 antibodies (Cisbio) overnight in dark at room temperature. When the donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC₅₀ determination was performed by fitting the curve of percent inhibition versus the log of the inhibitor concentration using Dotmatics.

### KRAS G12D Protein Preparation and Crystallization

### KRAS G12D Protein Purification

KRAS G12D) 1-169aa was cloned into the pET28a vector. Gene was placed in-frame with an N-terminal 6×his-tag and a Sumo tag. The construct was transformed into BL21(DE3) cells. Protein expression was induced when cells reached an OD₆₀₀ of 0.6, by addition of 1-thio-β-D-galactopyranoside (IPTG) to a final concentration of 200 μM followed by overnight incubation at 16° C. Bacteria were harvested by centrifugation (4000 rpm, 20 mins, 4)° C., 1 liter cell paste was resuspended in 30 ml of 50 mM Tris pH 8.0, 300 mM NaCl, 20 mM imidazole, 5 mM MgCl2 supplied with 2 piles of EDTA-free protease inhibitor cocktail table (Roche Diagnostics). Protein was purified with His-trap HP column (Cytiva) following standard protocols. The N-terminal His-sumo tag was cleaved by overnight digestion with ULP1 proteas, and UPL1, His-sumo tag were removed by reload into His-trap HP column (Cytiva). Protein was further purified by gel filtration using Hiload 16/600 Superdex 75 pg (Cytiva)equilibrated with 20 mM Tris pH 8.0, 100 mM NaCl, 5 mM MgCl2. Protein solution was concentrated to 30-40 mg/ml for crystallization trials.

### KRAS G12D Crystallization

KRAS G12D with small molecule inhibitor co-crystals were grown at 20° C. by mixing 1 μl of protein (40 mg/ml) with an equal volume of crystallization buffer using sitting drop vapor diffusion. Crystals appeared in drops containing 1.0M LiCl, 0.1M Citric acid pH 5.0, 20% PEG 6000. Diffraction data were collected at beamlines BL10U2 at Shanghai Synchrotron Radiation Facility.

### Metabolic Stability in Different Species of Liver Microsome

Liver microsomes were first mixed with NADPH to obtain final concentrations of microsomes and NADPH of 0.5 mg/mL and 1 mM, respectively. Test compounds was added to incubation system at final concentration of 1 μM and incubated at 37° C. The incubation was initiated by the addition of NADPH into the system. Aliquots of 20 μL were taken from the incubation system at 0, 15, 30, 45 and 60 min after the initiation of incubation. The reaction solutions were stopped by the addition of cold acetonitrile with analytical IS. Samples were centrifuged at 4000 rpm for 5 minutes and were then analyzed on LC-MS/MS.

Peak areas of samples from various timepoints were determined from extracted ion chromatograms; and were then plotted to calculate metabolic stability. The slope value, k, was determined by linear regression of the natural logarithm of the remaining percentage of the parent drug vs. incubation time curve. The in vitro half-life (in vitro t_{1/2}) was determined from the slope value:

$$in vitro {t}_{1 / 2} = - ( 0.693 / k )$$

Conversion of the in vitro t_{1/2} (in min) into the in vitro intrinsic clearance (in vitro CLᵢₙₜ, in μL/min/mg proteins) was done using the following equation (mean of duplicate determinations):

$$in vitro C \text{?} = \frac{0.693}{{t}_{1 / 2}} \times \frac{volume of incubation ( μ L )}{amount of proteins ( mg )}$$

$$\text{?} \text{indicates text missing or illegible when filed}$$

The control compound (verapamil) was included in the assay to ensure the data consistency. The negative control (identical experimental set-up but no NADPH in the incubation system) was used to exclude the misleading factor that resulted from instability of chemical itself.

### CYP (Cytochrome P450) Enzymes Inhibition Assay in Human Liver Microsome

The incubation was carried out in 96-well plates. 1μ L of test compound working solution or vehicle was added into 179μ L of human liver microsomes fortified with substrates of CYP1A2 (40 μM phenacetin), 2C₉ (6 μM diclofenac), 2019 (50 μM (S)-mephenytoin), 2D₆ (10 μM dextromethorphan) and 3A4 (1 μM midazolam or 50 μM testosterone). The incubation plate was pre-warmed at 37° C. for 5 min in water bath before the reactions are started by the addition of 20μ L of 10 mM NADPH solution. The reaction was carried out in the 37° C.-water bath.

At the predetermined time points, the reaction was stopped by adding 300 μL of quenching solution (acetonitrile with internal standards) to each well. The sample plate was vortexed for 1 min and centrifuged at 3000 g for 10 min. 100 μL of the supernatant was transferred to a new 96-well plate then mixed with 100 μL water for analysis by LC-MS/MS followed by data processing (i.e., percent inhibition at 10 μM or IC₅₀ determination).

### Time Dependent Cytochrome P450 (CYP) Enzymes Inhibition Assay (TDI) in Human Liver Microsome

The TDI assay involves pre-incubation (“inactivation incubation”) of 0.1 mg· mL-1 human liver microsome with 10 μM test compounds and Positive Control in the presence or absence of 1 mM NADPH at 37° C. for 30 min. Following the pre-incubation period, remaining CYP activity was determined by subsequently adding substrates (1A2, 40 μM phenacetin; CYP2B6,50 UM bupropion; CYP2C8, 5 μM paclitaxel; CYP2C9, 6 μM diclofenac; CYP2C19, 50 μM (S)-mephenytoin; CYP2D6, 10 μM dextromethorphan. CYP3A. 1 μM Midazolam or 50 μM Testosterone) and NADPH to the pre-incubation mixtures and an “activity incubation” was done for another 20 min for CYP1A2,2B6,2C19,2D6, 10 min for CYP2C8, CYP3A (testosterone), 6 min for CYP2C9 and 5 min for 3A (midazolam). All reactions are terminated by the addition of ice-cold acetonitrile with internal standard and then centrifuge for LC-MS/MS analysis.

### Bidirectional Permeability Assay in MDCKII-MDR1 Cell Monolayer

MDCKII-MDR1 cells were first prepared in cell seeding medium. 50 μL of cultured cell suspension was added to each well of a previously prepared Transwell plate. Incubate the plate for 4-8 days. Replace the medium every other day. The integrity of cell monolayer was assessed via electrical resistance method prior to permeability measurement.

To determine the rate of drug transport in the apical to basolateral direction. 125 μL of test compound working solution were added to the Transwell insert (apical compartment), and transferred 50 μL sample (DO sample) immediately from the apical compartment to a new 96-well plate. To determine the rate of drug transport in the basolateral to apical direction. 285 μL of working solution of compounds are added to the receiver plate wells (basolateral compartment), and transfer 50 μL sample (DO sample) immediately from the basolateral compartment to a new 96-well plate. The plates are incubated at 37° C. for 2 hours. At the end of the transport period, transfer 50 μL directly from the apical and basolateral wells and transfer to a new plate. Then add 200 μL of cold acetonitrile containing internal standards (IS: 2 μM ketoprofen, 200 nM labetalol, 200 nM caffeine and 100 nM alprazolam) into the plate. Vortex for 5 minutes. Samples are centrifuged at 3,220 g for 20 minutes. Aliquot of 100 L of the supernatant is diluted by 100 μL ultra-pure H₂O, and the mixture is used for LC/MS/MS analysis. All incubations are performed in duplicate. The apparent permeability (Papp), in units of centimeter per second, can be calculated for MDCKII-MDR1 drug transport assays using the following equation:

$${P}_{app} = \frac{{V}_{A}}{Area \times time} \times \frac{{[ drug ]}_{acceptor}}{{[ drug ]}_{initial , donor}}$$

Where V_{A} is the volume (in mL) in the acceptor well (0.235 mL for Ap→Bl flux and 0.075 mL for Bl→Ap flux), Area is the surface area of the membrane (0.143 cm² for Transwell-96 Well Permeable Supports), and time is the total transport time in seconds.

The efflux ratio can be determined using the following equation:

$$Efflux Ratio = \frac{{P}_{app ( B - A )}}{{P}_{app ( A - B )}}$$

Where P_{app(B-A)} indicates the apparent permeability coefficient in basolateral to apical direction, and P_{app(A-B)} indicates the apparent permeability coefficient in apical to basolateral direction.

The recovery can be determined using the following equation:

$$Recovery \% = \frac{{[ drug ]}_{acceptor} \times {V}_{A} + {[ drug ]}_{donor} \times {V}_{D}}{{[ drug ]}_{initial , donor} \times {V}_{D}} \times 100$$

Where V_{A} is the volume (in mL) in the acceptor well (0.235 mL for Ap→Bl flux, and 0.075 mL for Bl→Ap), V_{D} is the volume (in mL) in the donor well (0.075 mL for Ap→Bl flux, and 0.235 mL for Bl→Ap).

### Intrinsic Clearances in Different Species of Hepatocytes

Prepare 10 mM stock solutions of test compounds and positive control in appropriate solvent (DMSO). Place incubation medium (William's E Medium supplemented with GlutaMAX) in a 37° C. water bath, and allow warming for at least 15 minutes prior to use. In separate conical tubes, dilute the 10 mM test compound and the positive control to 100 μM by combining 198 μL of 50% acetonitrile 50% water and 2 μL of 10 mM stock. Pipette 198 μL of cryopreserved hepatocytes (0.5×10′ viable cells/mL) into each wells of a 96—well non—coated plate. Pipette 2 μL of the 100 μM test compounds or positive control into respective wells of the 96—well non—coated plate to start the reaction. The final concentration of test compound or control compounds is 1 μM. Return the plate to the incubator and place on an orbital shaker. Remove well contents in 25 μL aliquots at time points of 0, 15, 30, 60, 90 and 120 minutes. The aliquots are then mixed with 6 volumes (150 μL) of cold acetonitrile with IS (2 μM ketoprofen, 200 nM labetalol, 200 nM caffeine and 100 nM alprazolam) to terminate the reaction. Centrifuge for 30 minutes at 3,220 g. Aliquots of 100 μL of the supernatants will be used for LC/MS MS analysis. The supernatant may be diluted with ultrapure water according to the LC-MS signal response and peak shape. All incubations will be performed in duplicate.

All calculations are carried out using Microsoft Excel. Peak areas are determined from extracted ion chromatograms. Determine the in vitro half-life (t_{1/2}) of parent compound by regression analysis of the percent parent disappearance vs. time curve.

The in vitro half-life (in vitro t_{1/2}) is determined from the slope value:

$$in vitro {t}_{1 / 2} = 0.693 / k$$

Conversion of the in vitro t_{1/2} (in min) into the in vitro intrinsic clearance (in vitro CLᵢₙₜ, in μL/min 10⁶ cells) is done using the following equation:

$$in vitro {CL}_{int} = kV / N$$

V=incubation volume (0.2 mL); N=number of hepatocytes per well (0.1×10⁶ cells).

### Mouse and Rat PK Study

The pharmacokinetics of compounds were evaluated in male CD-1 mice or SD-JVC rats via intravenous and oral administration. For intravenous administration study, test compounds were dissolved in DMA: 30% Solutol HIS 15(w/v): Saline (20:20:60, by volume) and injected with a 1 mg/kg dose via tail vein. For oral administration study, test compounds were dissolved in 0.5% MC or PEG400 Phosal 50 PG/EtOH (30/60/10, by volume) and administrated to mice at 10 mg/kg or 30 mg/kg by gavage. Animals will be grouped and treated according to body weight. At the time points after dosing (5 (IV only). 15, and 30 min and 1, 2, 4, 8 and 24 h after administration), Rat blood samples will be collected from JVC, Mice will be anesthetized by isoflurane and blood samples will be collected from orbital bleeding. Blood samples will be collected into 1.5 mL EDTA.K2 coated EP tube. Approximately 50 μL blood (Mouse) and 150 μL blood (Rat) were collected at each time point and placed on ice, then centrifuge at 5600 rpm 7 min at 4° C. to obtain plasma. Plasma will be transferred into new tube and stored at −20° C. or dry ice temporary. The samples will be stored at −80° C. until ex vivo PK assay.

Plasma concentrations were determined via the following sample processing method and measurement conditions. An aliquot of 10 μL sample was added with 200 μL IS (Terfenadine, 5 ng/ml) in ACN. The mixture was vortexed for 1 min, and centrifuged at 4000 rpm for 10 min at 4° C. An aliquot of 80 μL supernatant was diluted with 80 μL water, and the mixed sample was injected to liquid chromatography-tandem mass spectrometry (LC-MS/MS, Triple Quad 5500) for analysis. Injected sample amount: 2 μL. Monitor: MRM; Column: Advanced Materials Technology, HALO AQ-C18 2.7 μm 90 Å, 50*2.1 mm; Column temperature: 40° C.; Mobile phase A: H₂O -0.1% FA, Mobile phase B: ACN-0.1% FA, Gradient program: 15% B-15% B (0 min-0.3 min), 15% B-90% B (0.3 min-1.0 min), 90% B-90% B (1.0 min-1.8 min), 90% B-30% B (1.8 min-2.0 min), 30% B-30% B (2.0 min-2.5 min).

### Sw1990 Pd Studies:

Female NCG mice were subcutaneously implanted with 5×10⁶ SW1990 cells per 200 μL PBS/matrigel in the right flank. After inoculation, when tumors reached a mean volume of approximately 350-450 mm³ in size, mice were randomized into treatment groups. Randomized mice would receive a single dose of vehicle consisting of 0.5% MC or test compounds at various dose (e.g. 30 or 100 mg/kg) by oral administration. Plasma was collected at 0.5, 2, 4, and 7 hours, and tumor was collected at 7 hours after dosing to determine exposure levels. Tumor fragments were snap frozen in homogenization tubes with liquid nitrogen and homogenized with T-PER Tissue Protein Extraction Buffer with protease and phosphatase inhibitors added fresh before use. Tumor lysates were then analyzed for ERK1/2 phosphorylation.

### SW1990 Efficacy Studies:

Female NCG mice were subcutaneously implanted with 5×10⁶ SW 1990 cells per 200 μL PBS/matrigel in the right flank. After inoculation, when tumors reached a mean volume of approximately 150-250 mm³ in size, mice were randomized into treatment groups. Randomized mice would receive vehicle consisting of 0.5% MC or test compounds at various dose (e.g. 30 or 100 mg/kg BID) by oral administration. Animals were monitored daily, tumor volumes were determined twice weekly in two dimensions using a caliper, and were expressed in mm³ using the formula: V=0.5(a×b²) where a and b are the long and short diameters of the tumor, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated t 0 )}{( placebo t ) - ( placebo t 0 )} ) )$$

treated t=treated tumor volume at time t
treated t₀=treated tumor volume at time 0
placebo t=placebo tumor volume at time t
placebo t₀=placebo tumor volume at time 0)
SW620 PD studies:

Female NOD/SCID mice were subcutaneously implanted with 5×10⁶ SW620 cells per 200 μL PBS/matrigel in the right flank. After inoculation, when tumors reached a mean volume of approximately 350-450 mm³ in size, mice were randomized into treatment groups. Randomized mice would receive a single dose of vehicle consisting of 0.5% MC or test compounds at various dose (e.g. 30 or 100 mg kg) by oral administration. Plasma was collected at 0.5, 2, 4, and 8 hours, and tumor was collected at 4 and 8 hours after dosing to determine exposure levels. Tumor fragments were snap frozen in homogenization tubes with liquid nitrogen and homogenized with T-PER Tissue Protein Extraction Buffer with protease and phosphatase inhibitors added fresh before use. Tumor lysates were then analyzed for ERK ½ phosphorylation.

### SW620 Efficacy Studies:

Female NOD/SCID mice were subcutaneously implanted with 5×10⁶ SW620 cells per 200 μL PBS/matrigel in the right flank. After inoculation, when tumors reached a mean volume of approximately 150-250 mm³ in size, mice were randomized into treatment groups. Randomized mice would receive vehicle consisting of 0.5% MC or test compounds at various dose (e.g. 30 or 100 mg/kg BID) by oral administration. Animals were monitored daily, tumor volumes were determined twice weekly in two dimensions using a caliper, and were expressed in mm³ using the formula: V=0.5(a× b²) where a and b are the long and short diameters of the tumor, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using the following formula:

$$\% growth inhibition = 100 \times ( 1 - ( \frac{( treated t ) - ( treated t 0 )}{( placebo t ) - ( placebo t 0 )} ) )$$

treated t=treated tumor volume at time t
treated t₀=treated tumor volume at time 0
placebo t=placebo tumor volume at time t
placebo t₀=placebo tumor volume at time 0
RKN PD studies:

Female NOD/SCID mice were subcutaneously implanted with 5×10⁶ RKN cells per 200 μL PBS/matrigel in the right flank. After inoculation, when tumors reached a mean volume of approximately 350-450 mm³ in size, mice were randomized into treatment groups. Randomized mice would receive a single dose of vehicle consisting of 0.5% MC or test compounds at various dose (e.g. 30 or 100 mg/kg) by oral administration. Plasma was collected at 0.5, 2, 4, and 8 hours, and tumor was collected at 4 and 8 hours after dosing to determine exposure levels. Tumor fragments were snap frozen in homogenization tubes with liquid nitrogen and homogenized with T-PER Tissue Protein Extraction Buffer with protease and phosphatase inhibitors added fresh before use. Tumor lysates were then analyzed for ERK1/2 phosphorylation.

### SW620 Efficacy Studies:

Female NOD/SCID mice were subcutaneously implanted with 5×10⁶ RKN cells per 200 μL PBS/matrigel in the right flank. After inoculation, when tumors reached a mean volume of approximately 150-250 mm³ in size, mice were randomized into treatment groups. Randomized mice would receive vehicle consisting of 0.5% MC or test compounds at various dose (e.g. 30 or 100 mg/kg BID) by oral administration. Animals were monitored daily, tumor volumes were determined twice weekly in two dimensions using a caliper, and were expressed in mm³ using the formula: V=0.5(a× b²) where a and b are the long and short diameters of the tumor, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume #standard error of the mean (SEM). Tumor growth inhibition (TGI) is calculated using the following formula:

$$\% growth inhibition = 100 \times ( 1 - ( \frac{( treated t ) - ( treated t 0 )}{( placebo t ) - ( placebo t 0 )} ) )$$

treated t=treated tumor volume at time t
treated t₀=treated tumor volume at time 0
placebo t=placebo tumor volume at time t
placebo t₀=placebo tumor volume at time 0
AsPC-1 PD studies:

Female BALB/c Nude mice were subcutaneously implanted with 3×10⁶ AsPC-1 cells per 200 μL PBS/matrigel in the right flank. After inoculation, when tumors reached a mean volume of approximately 350-450 mm³ in size, mice were randomized into treatment groups. Randomized mice would receive a single dose of vehicle consisting of 0.5% MC or test compounds at various dose (e.g. 30 or 100 mg kg) by oral administration. Plasma was collected at 0.5, 2, 4, and 8 hours, and tumor was collected at 4 and 8 hours after dosing to determine exposure levels. Tumor fragments were snap frozen in homogenization tubes with liquid nitrogen and homogenized with T-PER Tissue Protein Extraction Buffer with protease and phosphatase inhibitors added fresh before use. Tumor lysates were then analyzed for ERK ½ phosphorylation.

### AsC-1 Efficacy Studies:

Female BALB/c Nude mice were subcutaneously implanted with 3×10⁶ AsPC-1 cells per 200 μL PBS/matrigel in the right flank. After inoculation, when tumors reached a mean volume of approximately 150-250 mm³ in size, mice were randomized into treatment groups. Randomized mice would receive vehicle consisting of 0.5% MC or test compounds at various dose (e.g. 30 or 100 mg/kg BID) by oral administration. Animals were monitored daily, tumor volumes were determined twice weekly in two dimensions using a caliper, and were expressed in mm³ using the formula: V=0.5(a× b²) where a and b are the long and short diameters of the tumor, respectively. Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume=standard error of the mean (SEM). Tumor growth inhibition (TGI) is calculated using the following formula:

$$\% growth inhibition = 100 \times ( 1 - ( \frac{( treated t ) - ( treated t 0 )}{( placebo t ) - ( placebo t 0 )} ) )$$

treated t=treated tumor volume at time t
treated t₀=treated tumor volume at time 0
placebo t=placebo tumor volume at time t
placebo t₀=placebo tumor volume at time 0
hERG Assay

hERG (the human Ether-à-go-go-Related Gene) encodes the rapidly activating potassium channel (I_{Kr}) contributing to the repolarization of the cardiac action potential. The blockade of hERG channel can lead to a QT prolongation in the electrocardiogram known as long QT syndrome. Drug-induced delayed ventricular repolarization in some cases may trigger a fatal arrhythmias-torsional apical ventricular tachycardia. About 25-40% of the leading drug compounds show varied extent of hERG dependent potential risks, and many drugs are withdrawn from the market due to the risk of the QT interval prolongation.

Before testing hERG current, the blank control will be diluted with appropriate volume of extracellular solution to make control working solution. The positive control and test article stock solutions will be taken from −20° C., thawed, and diluted with an appropriate volume of extracellular solution to make the working solution.

The working solution for test article at highest concentration will be diluted with extracellular solution from the stock solution or the stock solution should be diluted with DMSO firstly. For other test concentrations of the test article, serial dilutions will be made using DMSO and then be prepared to the working solutions with extracellular solution. The DMSO concentration in the final working solutions will be 0.3%. The specific preparation information will be recorded in the compound working-solution preparation form. Finally, all the working solutions of test article will be ultrasonicated for 20 minutes before performing the patch clamp experiment.

The blank control (DMSO) stock solution will be kept at room temperature. The blank control working solution will be prepared on the test day and kept at room temperature. The positive control stock solution and the test article stock solution will be kept at −20° C. The positive control and the test article working solutions will be prepared on the test day and kept at room temperature.

The test articles concentrations will be automatically set for 30, 10, 3, 1 and 0.3 μM. The blank control will be 0.3% DMSO, and the positive control (Cisapride) concentrations will be 1000, 100, 10, 1,0.1 nM.

Automated Patch Clamp system QPatch 48× (Sophion) will be used for electrophysiological recording in this study.

Place the prepared cells on the centrifuge of the Qpatch work plane, wash the cells with multiple centrifugation/suspension times, and replace the cell culture medium with extracellular solution. Take out an MTP-96 plate and place it on the MTP source position. A QPlate chip will be took out and put in the Qplate source position. The barcode reader scans the barcode of MTP-96 board and QPlate chip and the gripper arm grab them to the measurement position. The intracellular and extracellular solution from the saline reservoir will be added to the intracellular saline well, cell and compound well of the QPlate chip. For the measuring, all the measuring points of QPlate will be under the initial quality control. The quality control process includes sucking the cell suspension from the cell container of the centrifuge, positioning the cells on the chip hole by the pressure controller, establishing a high-resistance seal, and forming a whole-cell recording mode. Once a stable control current baseline is obtained, the test article will be applied to the cells by sequential aspiration from the MTP-96 plate in order of concentration. The hERG current will be recorded using the whole-cell patch clamp technique at a holding potential of −80 mV and then depolarized to −50 mV for 0.5 seconds to test the leak current. Then the voltage will be depolarized to 30 mV for 2.5 seconds. The peak tail current will be induced by a repolarizing pulse to −50 mV for 4 seconds. This protocol will be repeated at 10 s intervals to observe the effect of test article on hERG tail current. The data will be collected by QPatch screening station and stored in QPatch database server.

In the experiment, each drug concentration will be applied twice recording period of at least 5 min. The control and test solutions will be applied to the cells sequentially from low to high concentration. The current of each cell detected in the extracellular solution without compound will be used as its own blank control.

IC₅₀ value will be calculated, and dose-response curve will be fitted using non-linear regression equation above, where IC₅₀ is the half maximal inhibitory concentration. IC₅₀ calculation and curve-fitting will be performed using GraphPad Prism software.

### Activity Tables

Each of the compounds in Tables 4 and 5 was tested in one or more of the biochemical assays provided herein and was found to have activity therein.

TABLE 4

| | KRAS^{WT} Probe | KRAS^{G12V} Probe | |
|---|---|---|---|
| | Displacement | Displacement | SW620 pERK |
| Example No. | Assay IC50 (nM) | Assay IC50 (nM) | IC₅₀ (nM) |
| Example 1 | 11 | 3.2 | 624 |
| Example 2 | 4.0 | 3.6 | 255 |
| Example 3 | 29 | 15 | 402 |
| Example 4 | 7.3 | 8.1 | 549 |
| Example 5 | 1.3 | 0.87 | 288 |
| Example 6 | 5.0 | 1.8 | 63 |
| Example 7 | 5.1 | 2.5 | 586 |
| Example 8 | 16 | 9.9 | 716 |
| Example 9 | 10 | 5.2 | 249 |
| Example 10 | 3.0 | 1.4 | 103 |
| Example 11 | 16 | 16 | 624 |
| Example 12 | 4.2 | 3.2 | 152 |
| Example 13 | 1.5 | 1.4 | 141 |
| Example 14 | 30 | 13 | 414 |
| Example 15 | 13 | 8.6 | 371 |
| Example 16 | 2.6 | 1.4 | 50 |
| Example 18 | 1.3 | 0.84 | 55 |
| Example 19 | 0.66 | 0.65 | 22 |
| Example 21 | 3.4 | 1.7 | 26 |
| Example 22 | 12 | 6.8 | 303 |
| Example 23 | 18 | 9.2 | 338 |
| Example 24 | 4.2 | 2.6 | 95 |
| Example 26 | 1.3 | 0.79 | 32 |
| Example 28 | 3.2 | 2.5 | 206 |
| Example 29 | 1.7 | 0.95 | 56 |
| Example 30 | 1.1 | 0.63 | 13 |
| Example 31 | 0.8 | 0.51 | 32 |
| Example 32 | 11 | 3.4 | 133 |
| Example 33 | 0.53 | 0.39 | 17 |
| Example 34 | 1.0 | 0.59 | 13 |
| Example 35 | 0.76 | 0.47 | 26 |
| Example 45 | 4.6 | 2.7 | 82 |
| Example 46 | 0.58 | 0.48 | 37 |
| Example 47 | 0.84 | 0.61 | 73 |
| Example 49 | 0.57 | 0.54 | 8.4 |
| Example 50 | 0.61 | 0.61 | 28 |
| Example 51 | 0.67 | 0.5 | 9.3 |
| Example 52 | 8.1 | 6.5 | 158 |
| Example 53 | 1.9 | 0.63 | 26 |
| Example 54 | 3.4 | 1.7 | 51 |
| Example 55 | 0.87 | 0.57 | 41 |
| Example 56 | 2.0 | 1.2 | 33 |
| Example 57 | 2.4 | 0.83 | 41 |
| Example 58 | 0.63 | 0.49 | 29 |
| Example 59 | 0.58 | 0.42 | 7.3 |
| Example 60 | 1.6 | 0.63 | 20 |
| Example 61 | 0.81 | 0.8 | 22 |
| Example 62 | 0.54 | 0.25 | 5.5 |
| Example 63 | 2.7 | 1.1 | 25 |
| Example 64 | 0.23 | 0.25 | 6.4 |
| Example 65 | 0.61 | 0.47 | 8.9 |
| Example 66 | 0.58 | 0.47 | 5.8 |
| Example 67 | 0.87 | 0.65 | 12 |
| Example 68 | 0.93 | 1.8 | 51 |
| Isomer 1 | | | |
| Example 68 | 0.69 | 0.51 | 1.4 |
| Isomer 2 | | | |

TABLE 5

| | KRAS^{WT} Probe | KRAS^{G12D} Probe | |
|---|---|---|---|
| | Displacement | Displacement | AsPC-1 pERK |
| Example No. | Assay IC50 (nM) | Assay IC50 (nM) | IC₅₀ (nM) |
| Example 2 | 4.0 | 6.2 | 594 |
| Example 10 | 3.0 | 1.1 | 123 |
| Example 17 | 188 | 9.6 | |
| Example 20 | 13 | 0.43 | 1010 |
| Example 27 | 2.9 | 0.95 | 836 |
| Example 30 | 1.0 | 0.31 | 11 |
| Example 31 | 0.8 | 0.43 | 40 |
| Example 34 | 1.0 | 0.59 | 25 |
| Example 35 | 0.76 | 0.33 | 22 |
| Example 36 | 30 | 1.1 | 2165 |
| Example 37 | 8.7 | 2.3 | 639 |
| Example 38 | 10 | 0.38 | 102 |
| Example 39 | 170 | 3.9 | 1782 |
| Example 40 | 166 | 7.6 | 2004 |
| Example 41 | 270 | 34 | |
| Example 42 | 1.4 | 0.17 | 141 |
| Example 43 | 2.1 | 0.73 | 92 |
| Example 44 | 3.4 | 0.65 | 184 |
| Example 48 | 28 | 20 | 3383 |
| Example 49 | 0.57 | 0.27 | 7.6 |
| Example 51 | 0.67 | 0.28 | 30 |
| Example 59 | 0.58 | 0.32 | 16 |
| Example 61 | 0.81 | 0.57 | 23 |
| Example 62 | 0.54 | 0.23 | 12 |
| Example 64 | 0.23 | 0.10 | 7.9 |
| Example 65 | 0.61 | 0.27 | 17 |
| Example 68 | 0.69 | 0.14 | 6.9 |
| Isomer 2 | | | |

As demonstrated by the data in Tables 4-5, the inventors surprisingly and unexpectedly discovered that the exemplary compounds in Tables 4-5 modulate or inhibit the activity of KRAS G12D and or G12V.

A number of references have been cited, the disclosures of which are incorporated herein by reference in their entirety.

1-71. (canceled)

72. A compound having Formula (I): 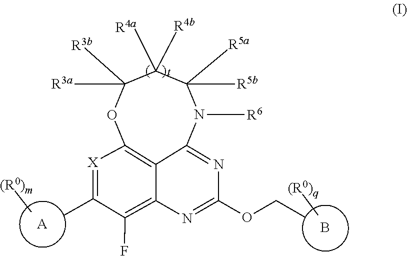
or a pharmaceutically acceptable salt, or tautomer thereof,
wherein:
ring A is 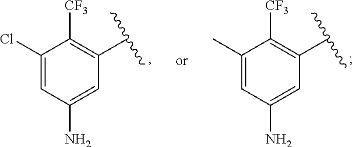 [SMILES: *c1cc(N)cc(Cl)c1C(F)(F)F ; *c1cc(N)cc(C)c1C(F)(F)F]
ring B is 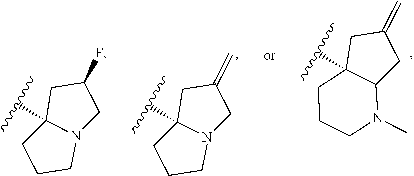
X is N;
each of R⁰ is hydrogen;
R⁶ is methyl, or ethyl;
t is 0 or 1;
each of m, and q is, independently, an integer from 0 to the maximum number of the substituent groups allowed on rings A, and B, respectively; and
each of R^{3a}, R^{3b}, R^{4a}, R^{4b}, R^{5a}, and R^{5b}, independently, is H, or
optionally, R^{5a}, and R^{5b}, together with the atom to which they are attached, form unsubstituted or substituted cyclopropyl, or unsubstituted or substituted oxetanyl; or
the R^{3a}, and R^{5a} groups, together with the atoms to which they are attached, form unsubstituted or substituted tetrahydro-2H-pyranyl; or
the R^{4a}, and R^{5a} groups, together with the atoms to which they are attached, form unsubstituted or substituted tetrahydrofuranyl.

73. The compound of claim 72, wherein t is 1.

74. The compound of claim 73, wherein R^{5a}, and R^{5b}, together with the atom to which they are attached, form cyclopropyl, or oxetanyl.

75. The compound of claim 72, wherein the compound is

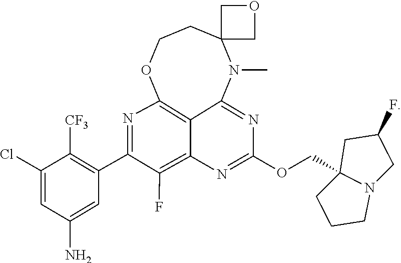 [SMILES: CN1c2nc(OC[C@@]34CCCN3C[C@H](F)C4)nc3c(F)c(-c4cc(N)cc(Cl)c4C(F)(F)F)nc(c23)OCCC12COC2]

76. The compound of claim 72, wherein the compound is

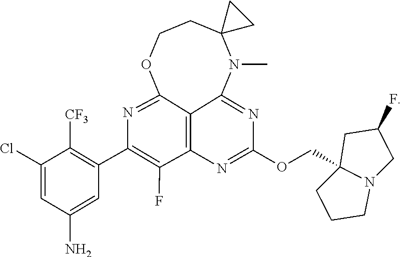 [SMILES: CN1c2nc(OC[C@@]34CCCN3C[C@H](F)C4)nc3c(F)c(-c4cc(N)cc(Cl)c4C(F)(F)F)nc(c23)OCCC12CC2]

77. The compound of claim 72, wherein the compound is

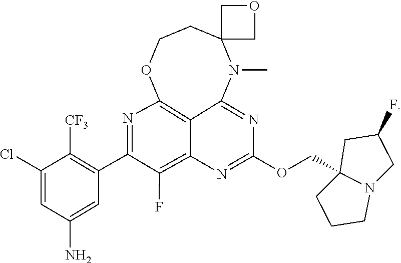 [SMILES: CN1c2nc(OC[C@@]34CCCN3C[C@H](F)C4)nc3c(F)c(-c4cc(N)cc(Cl)c4C(F)(F)F)nc(c23)OCCC12COC2]

78. The compound of claim 73, wherein the R^{4a}, and R^{5a} groups, together with the atoms to which they are attached, form tetrahydrofuranyl.

79. The compound of claim 72, wherein the compound is

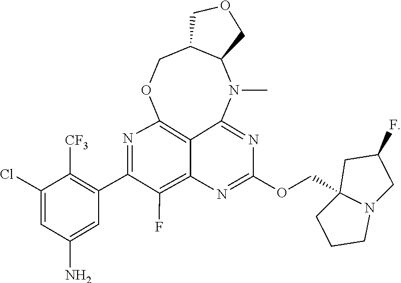 [SMILES: CN1c2nc(OC[C@@]34CCCN3C[C@H](F)C4)nc3c(F)c(-c4cc(N)cc(Cl)c4C(F)(F)F)nc(c23)OC[C@@H]2COC[C@H]21]

80. The compound of claim 72, wherein the compound is

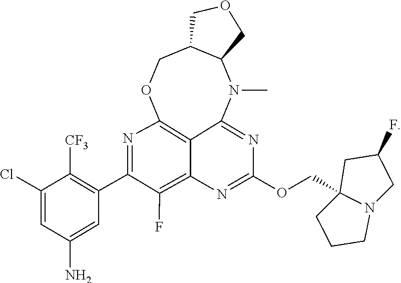 [SMILES: CN1c2nc(OC[C@@]34CCCN3C[C@H](F)C4)nc3c(F)c(-c4cc(N)cc(Cl)c4C(F)(F)F)nc(c23)OC[C@@H]2COC[C@H]21]

81. The compound of claim 72, wherein t is 0.

82. The compound of claim 78, wherein the R^{3a}, and R^{5a} groups, together with the atoms to which they are attached, form unsubstituted or substituted tetrahydro-2H-pyranyl.

83. The compound of claim 72, wherein the compound is

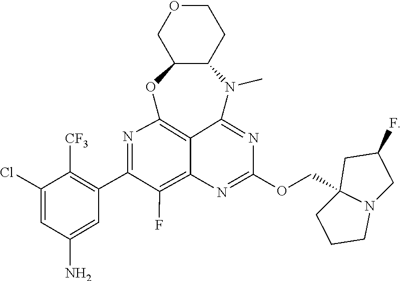 [SMILES: CN1c2nc(OC[C@@]34CCCN3C[C@H](F)C4)nc3c(F)c(-c4cc(N)cc(Cl)c4C(F)(F)F)nc(c23)O[C@H]2COCC[C@@H]21]

84. The compound of claim 72, wherein the compound is

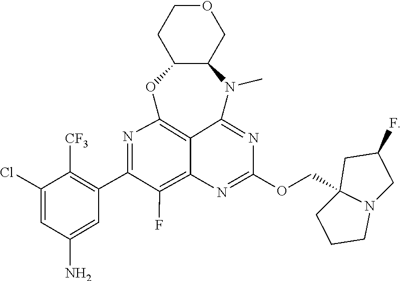 [SMILES: CN1c2nc(OC[C@@]34CCCN3C[C@H](F)C4)nc3c(F)c(-c4cc(N)cc(Cl)c4C(F)(F)F)nc(c23)O[C@@H]2CCOC[C@H]21]

85. The compound of claim 72, wherein the compound is

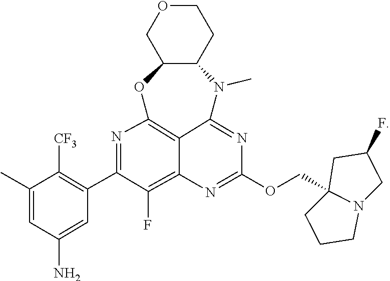 [SMILES: Cc1cc(N)cc(-c2nc3c4c(nc(OC[C@@]56CCCN5C[C@H](F)C6)nc4c2F)N(C)[C@H]2CCOC[C@@H]2O3)c1C(F)(F)F]

86. The compound of claim 72, wherein the compound is

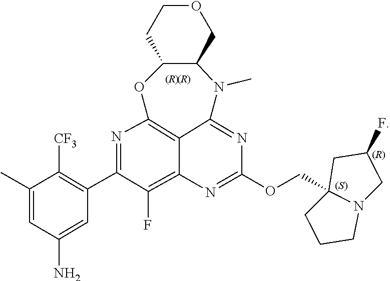

87. The compound of claim 72, wherein the compound is

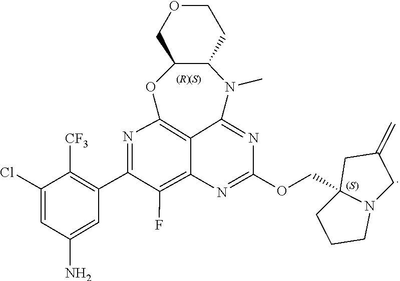

88. The compound of claim 72, wherein the compound is

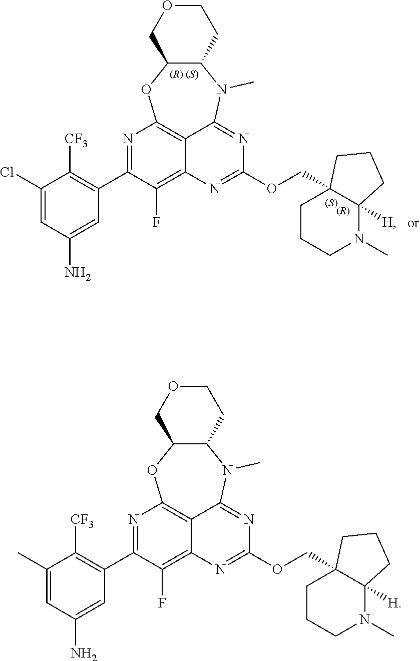

89. A pharmaceutical composition comprising an effective amount of a compound of claim 72, or a pharmaceutically acceptable salt, or tautomer thereof, and a pharmaceutically acceptable carrier, excipient or vehicle.

90. A method for inhibiting the activity of KRAS mutant protein or KRAS amplification in a cell, comprising contacting said cell with an effective amount of a compound of claim 72, or a pharmaceutically acceptable salt, or tautomer thereof, optionally wherein the KRAS mutant protein is KRAS G12D and/or G12V mutant protein.

91. A method for treatment or prevention of cancer, the method comprising administering to a subject in need thereof an effective amount of a compound of claim 72, or a pharmaceutically acceptable salt, or tautomer thereof, wherein the cancer is mediated by KRAS G12D and/or G12V mutation.
